SEQUENCE LISTING (517 sequences)

SEQ ID NO: 1 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - protein, 457 aa, synthetic construct
MPNTNTTVVRRRRIILFPVPFPGHLNPMLQLANVLYRKGFSITIFHTNFNKPKTSNYPHFTFRFILDNDPQDEWLRNLPTTGPGVGARIPVINKHGADEFRKELELCMLASPSDEEVACLITDALWYFAQPVADSLNLKRLVLQTGSLFNFHCLVSLPKFDELGYLDPEDKTRLDEPASGFPMLKVKDIRSAYSHIQESKPILMKMIKQTRASSGVIWNSFKELEESELETIQREIPAPSFLIPLPKHLRASSTSLLDTDRSTFQWLDQQPPSSVLYVGFGSQSSLDPKDFLEIARGLVDSKQSFLWLVRPGFVKGYEWVEPLPDGFLGEKGRIVKSAPQQEVLAHGAIGAFWTHGGWNGTLESVCEGVPMIFSDFGLDQPLNARYMSDVLHVGVYLENGWERGEIANAIRRVMVDEEGEVMRQNARVLKDKADRSLMPGGSSYESLESLQSYISSL

SEQ ID NO: 3 - protein, 457 aa, synthetic construct
MPNTNTTVVRRRRIIMFPVPFPGHLNPMLQLANVLYSKGFSITIFHTNFNKPKTSNYPHFTFRFILDNDPQDEWLRNLPTHGPGVGARIPVINEHGADEFRKELELLMLASPSDEEVSCVITDALWYFAQSVADSLNLKRLVLQTGSLFNFHCLVSLPKFDELGYLDPEDKHRLDEPAVGFPMLKVKDIKRAYSHWQESKPILGKMVKQTRASSGVIWNSAKELEESELETVQREIPAPSFLLPLPKHYRASSTSLLDTDRSVFQWLDQQPPSSVLYVSFGSQSELDPKDFLEIARGLVASKQSFLWLVRPGFVKGSTWIELLPDGFLGEKGRVVKSAPQQEVLAHGAIGAFWTHGGWNGTMESVCEGVPMIFSDFGLDQPLNARYMSEVLHVGVYLENGWERGEIANAVRRVMVDEEGEVMRQNARVLKQKADRSIAPGGSSYESLERLVSYISSL

SEQ ID NO: 4 - protein, 457 aa, synthetic construct
MPNTNTTVRRRRRIIMFPVPFPGHLNPMLQLANVLYSKGFSITIFHTNFNKPKTSNYPHFTFRFILDNDPQDEWLRNLPTTGPGVGARIPVINKHGADEFRKELELLMLASESDEEVSCLITDALWYFAQSVADSLNLKRLVLQTGSLFNFHCLVSLPKFDELGYLDPDDKTRLDEPAVGFPMLKVKDIKSAYSHIQESKPILMKMVKQTRASSGVIWNSAKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMESVCEGVPMIFSDFGLDQPLNARYMSDVLHVGVYLENGWERGEIANAVRRVMVDEEGEYMRQNARVLKQKADRSIMKGGSSYESLESLVSYISSL

SEQ ID NO: 5 - protein, 457 aa, synthetic construct
MPNTNTTVRRRRRVILFPVPFPGHINPMLQLANVLYRKGFSITIFHTNFNKPKTSNYPHFTFRFILDNDPQDEWLRNLPTTGPGVGARIPVINKHGADELRKELELCMRATPEDEEVACVITDALWYFAQSVADSLNLRRLVLQTGSLFNFHCLVSLPQFDELGYLDPEDKHRLEEQASGFPMLKVKDIKSAYSHIQESKPILMKMIEQTRASSGVIWNSFKELEESELETIIREIPAPSFLIPLPKHYTASSTSLLDTDRSTFQWLDQQPPSSVLYVSFGSQSELDPKDFLEIARGLVDSKQSFLWLVRPGFVKGYTWVELLPDGFLGERGRIVKSAPQQEVLAHKAIGAFWTHGGWNGTMESVCEGVPMIFSDFGLDQPLNARYMSDVLKVGVYLENGWERGEIANAIRRVMVDEEGEYMRQNARKLKQKADVSLMPGGSSYESLESLVSYISSL

SEQ ID NO: 6 - protein, 457 aa, synthetic construct
MPNTNTTVRRRRRIILFPVPFPGHLNPMLQLANVLYSKGFSITILHTNFNKPKTSNYPHFTFRFILDNDPQDEWLRNLPTHGPGVGARIPVINEHGADELRRELELLMLASEEDEEVSCVITDALWYFAQSVADSLNLKRLVLQTGSLFNFHCLVSLPKFDELGYLDPDDKHRLEEQASGFPMLKVKDIKSAYSHIQESKPILGKMIEQTRASSGVIWNSAKELEESELETVIREIPAPSFLIPLPKHLRASSTSLLDTDRTVFQWLDQQPPSSVLYVSFGSQSEVDPKDFLEIARGLVDSKQSFLWLVRPGFVKGYTWVELLPDGFLGEKGRIVKSAPQQEVLAHGAIGAFWTHGGWNGTLESVCEGVPMIFSDFGLDQPLNARYMSDVLHVGVYLENGWERGEIANAVRRVMVDEEGEYIRQNARVLKQKLDVSLAPGGSSYESLESLVSYISSL

SEQ ID NO: 7 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRRELELLMRESESDEEVSCLITDALWYFAQSVADSLNLRRLVLQTGSLFNFHCLVSLPKFDELGYLDPEDKHRLDEPAVGFPMLKVKDIKSAYSHIQESKPILGKMVKQTRASSGVIWNSFKELEESELETVQREIPAPSFLIPLPKHYTASSTSLLDTDRTVFQWLDQQPPSSVLYVSFGSQSEVDAKDFLEIARGLVDSKQSFLWLVRPGFVKGYTWVEPLPDGFLGEKGRVVKSAPQQEVLAHGAIGAFWTHGGWNGTLESVCEGVPMIFSDFGLDQPLNARYMSEVLKVGVYLENGWERGEIANAIRRVMVDEEGEYMRQNARVLKQKADRSLMKGGSSYESLESLVSYISSL

SEQ ID NO: 8 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPVINEHGADELRKELELLMLASEEDEEVSCLITDALWYFAQSVADSLNLRRLVLQTGSLFNFHCLVSLPKFDELGYLDPEDKTRLEEQASGFPMLKVKDIKSAYSHWQESKPILGKMVEQTKASSGVIWNSFKELEESELETVIREIPAPSFLIPLPKHLTASSTSLLDTDPTFFQWLDQQPPSSVLYVSFGSQSELDAKDFLEIARGLVDSKQSFLWLVRPGFVKGSTWVELLPDGFLGERGRIVKSAPQQEVLAHGAIGAFWTHGGWNGTLESVCEGVPMIFSDFGLDQPLNARYMSEVLKVGVYLENGWERGEIANAIRRVMVDEEGEYMRQNARVLKQKADVSLMKGGSSYESLESLVSYISSL

SEQ ID NO: 9 - protein, 457 aa, synthetic construct
MPNTNTTVRRRRRIILFPVPFPGHINPILQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKTRLEEQASGFPMLKVKDIKSAYSHWQESKPILGKMIEQTKASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGEKGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 10 - protein, 457 aa, synthetic construct
MPNTNTTTRRRRRVIMFPVPFPGHINPMLQLANVLYSRGFSITILHTNFNAPKTSNYPHFTFRFILDNDPQDEWLRNLPTTGPGVGARIPVINKHGADEFRDELELLMRDSPSDEEVSCLITDALWYFAQPVADSLNLPRLVLQTGSLFNFHCLVSLPLFDELGYLDPEDKHRLEEPVVGFPMLKVKDIRKAYSHIQESKPILGKMVKQTKASSGVIWNSFKELEESELETIQREIPAPSFLIPLPKHFTASSTSLLDTDRSTFPWLDQQPPSSVLYVSFGSQSSLDEKDFLEIARGLVDSKQSFLWLVRPGFVKGSEWIELLPDGFLGEKGRIVKSAPQQEVLAHKAIGAFWTHGGWNGTLESVCEGVPMICSDFGLDQPLNARYMSDVLKVGVYLENGFERGEIANAVRRVMVDEEGEVMRQNARKLKQKLDVSIMPGGSSYESLERLVSYISSL

SEQ ID NO: 11 - protein, 457 aa, synthetic construct
MPNTNTTTRRRRRVILFPVPFPGHINPMLQLANVLYSRGFSITILHTNFNAPKTSNYPHFTFRFILDNDPQDEWLRNLPTTGPGVGARIPVINKHGADEFRKELELLMSDTPSDEEVACLITDALWYFAQSVADSLNLPRLVLQTGSLFNFHCLVSLPLFDELGYLDPEDKHRLDEPVVGFPMLKVKDIRRAYSHSQESKPILSKMVEQTKASSGVIWNSFKELEESELETIQREIPAPSFLGPLPKHYTASSTSLLDTDRSFFPWLDQQPPSSVLYVSFGSQSSLDEKDFLEIARGLVDSKQSFLWLVRPGFVKGSEWIELLPDGFLGERGRIVKSAPQQEVLAHKAIGAFWTHGGWNGTLESVCEGVPMICSDFGLDQPLNARYMSDVLKVGVYLENGFERGEIANAIRRVMVDEEGEVMRQNARKLKQKLDVSIMPGGSSYESLERLVSYISSL

SEQ ID NO: 12 - protein, 457 aa, synthetic construct
MPNTNTTTRRRRRVILFPVPFPGHINPMLQLANVLYSRGFSITILHTNFNAPKTSNYPHFTFRFILDNDPQDEWLRNLPTTGPGVGARIPVINKHGADEFRDELELLMADSPSDEEVSCLITDALWYFAQSVADSLNLPRLVLQTGSLFNFHCLVSLPLFDELGYLDPEDKHRLEEQVVGFPMLKVKDIRKAYSHKQESKPILGKMVEQTKASSGVIWNSFKELEESELETIQREIPAPSFLLPLPKHFTASSTSLLDTDRSFFQWLDQQPPSSVLYVSFGSQSSLDEKDFLEIAHGLVDSKQSFLWLVRPGFVKGSEWIELLPDGFLGEKGRIVKSAPQQEVLAHEAIGAFWTHGGWNGTLESVCEGVPMICSPFGLDQPLNARYMSEVLKVGVYLENGFERGEIANAIRRVMVDEEGEEIRQNARVLKDKLDRSLMKGGSSYESLESLVSYISSL

SEQ ID NO: 13 - protein, 457 aa, synthetic construct
MPNTNTTTRRRRRVIMFPVPFPGHINPMLQLANVLYSRGFSITILHTNFNAPKTSNYPHFTFRFILDNDPQDEWLRNLPTTGPGVGARIPVINQHGADEFRRELEQLMRDTPEDEEVACLITDALWYFAQSVADSLNLPRLVLQTGSLFNFHCLVSLPLFDELGYLDPEDKHRLEEQVVGFPMLKVKDIKKAYSHDQESKPILGKMVKQTRASSGVIWNSFKELEESELETIQREIPAPSFLIPLPKHYTASSTSLLDTDRSFFQWLDQQPPKSVLYVSFGSQSSVDEKDFLEIARGLVDSKQSFLWLVRPGFVKGSEWIELLPDGFLGEKGRIVKSAPQQEVLAHKAIGAFWTHGGWNGTLESVCEGVPMIFSPFGLDQPLNARYMSDVLKVGVYLENGFERGEIANAIRRVMVDEEGEEMRQNARVLKQKLDVSLMKGGSSYESLESLVSYISSL

SEQ ID NO: 14 - protein, 457 aa, synthetic construct
MPNTNTTTRRRRRVILFPVPFPGHINPMLQLANVLYSRGFSITILHTNFNAPKTSNYPHFTFRFILDNDPQDEWLRNLPTTGPGVGARIPVINQHGADEFRDELELLMADSPSDEPVSCLITDALWYFAQSVADSLNLPRLVLQTGSLFNFHCLVSLPLFDELGYLDPEDKHRLEEPVVGFPMLKVKDILKAYSHKQESKPILGKMVKQTKASSGIIWNSFKELEESELETIQREIPAPSFLIPLPKHFTASSTSLLDTDRSFFPWLDQQPPSSVLYVSFGSQSSVDEKDFLEIAHGLVDSKQSFLWLVRPGFVKGSEWLELLPDGFLGEKGRIVKSAPQQEVLAHEAIGAFWTHGGWNGTLESVCEGVPMIFSPFGLDQPLNARYMSDVLKVGVYLENGFERGEIANAIRRVMVDEEGEVIRENARVLKQKLDVSLMKGGSSYESLESLVSYISSL

SEQ ID NO: 15 - protein, 457 aa, synthetic construct
MPNTNTTVRRRRRIILFPVPFPGHINPMLQLANLLYSKGFSITILHTNFNAPKTSNYPHFTFRFILDNDPQDEWLRNLPTTGPGVGARIPVINKHGADELRDELEILMRDSPEDEPVSCVITDALWYFAQSVADSLNLPRLVLQTGSLFNFHCLASLPLFDELGYLDPEDKHRLEEPVVGFPMLKVKDIRKAYSHIDESKPILGKMVKQTKASSGVIWNSFKELEESELETIQREIPAPSFLIPLPKHFTASSTSLLDTDRSFFPWLDQQPPSSVLYVSFGSQSSVDEKDFLEIARGLVDSKQSFLWLVRPGFVKGSEWIELLPDGFLGERGRIVKSAPQQEVLAHEAIGAFWTHGGWNGTLESVCEGVPMICSPFGLDQPLNARYMSDVLKVGVYLENGFERGEIANAIRRVMVDEEGEEMRQNARVLKQKLDVSLMKGGSSYESLESLVSYISSL

SEQ ID NO: 16 - protein, 457 aa, synthetic construct
MPNTNTTVRRRRRVILFPVPFPGHINPMLQLANLLYSKGFSITILHTNFNAPKTSNYPHFTFRFILDNDPQDEWLRNLPTTGPGVGARIPVINKHGADELRDELELLMRSSPSDEEVSCLITDALWYFAQSVADSLNLPRLVLQTGSLFNFLCLASLPLFDELGYLDLEDKHRLEEQVVGFPMLKVKDIRKAYSHKDESKPILGKMVKQTKASSGVIWNSFKELEESELETIQREIPAPSFLIPLPKHFTASSTSLLDTDRTFFQWLDQQPPNSVLYVSFGSQSQVDEKDFLEIAHGLVDSKQSFLWLVRPGFVKGSEWIELLPDGFLGERGRIVKSAPQQEVLAHEAIGAFWTHGGWNGTLESVCEGVPMIFSPFGLDQPLNARYMSDVLKVGVYLENGWEREEIANAIRRVMVDEEGEEIRENARVLKQKLDVSLMKGGSSYESLESLVSYISSL

SEQ ID NO: 17 - protein, 457 aa, synthetic construct
MPNTNTTVRRRRRVILFPVPFPGHINPMLQLANLLYSKGFSITILHTNFNAPKTSNYPHFTFRFILDNDPQDEWLRNLPTHGPGVGARIPVINQHGADELRDELELLMASSPEDEPVSCLITDALWYFAQSVADSLNLPRLVLQTGSLFNFHCLASLPLFDELGYLDLDDKHRLEEQVVGFPMLKVKDILKAYSHKDESKPILGKMVKQTKASSGIIWNSFKELEESELETVQREIPAPSFLIPLPKHFTASSTSLLDTDRSFFPWLDQQPPSSVLYVSFGSQSQVDEKDFLEIAHGLVDSKQSFLWLVRPGFVKGSTWLEPLPDGFLGERGRIVKSAPQQEVLAHEAIGAFWTHGGWNGTLESVCEGVPMICSPFGLDQPLNARYMSEVLKVGVYLENGFEREEIANAIRRVMVDEEGEEIRERARVLKQKLDVSLMKGGSSYESLESLVSYISSL

SEQ ID NO: 18 - protein, 457 aa, synthetic construct
MPNTNTTTRRRRRVIMFPVPFPGHLNPMLQLANVLYSKGFSITILHTNFNAPKTSNYPHFTFRFILDNDPQDEWLRNLPTTGPGVGARIPVINKHGADEFRDELELLMRDSPSDEEVSCLITDALWYFAQSVADSLNLPRLVLQTGSLFNFHCLVSLPLFLELGYLDPEDKHRLDEPVVGFPMLKVKDIRKAYSHIQESKPILGKMVKQTRASSGVIWNSFKELEESELETIQREIPAPSFLIPLPKHFTASSTSLLDTDRSFFQWLDQQPPSSVLYVSFGSQSSLDEADFLEIARGLVDSKQSFLWLVRPGFVKGSEWIELLPDGFLGEKGRIVKSAPQQEVLAHEAIGAFWTHGGWNGTLESVCEGVPMIFSPFGLDQPLNARYMSDVLKVGVYLENGFERGEIANAVRRVMVDEEGEVMRQNARVLKDKLDVSLAKGGSSYESLESLVSYISSL

SEQ ID NO: 19 - protein, 457 aa, synthetic construct
MPNTNTTTRRRRRVIMFPVPFPGHLNPMLQLANVLYSKGFSITILHTNFNAPKTSNYPHFTFRFILDNDPQDEWLRNLPTTGPGVGARIPVINKHGADEFRDELEKLLRDSPDDEEVSCLITDALWYFAQSVADSLNLPRLVLQTGSLFNFHCLVSLPLFLELGYLDPEDKHRLDEPVVGFPMLKVKDIRKAYSHSQESKPILMKMVKQTRASSGVIWNSFKELEESELETIQREIPAPSFLIPLPKHFTASSTSLLDTDRSAFQWLDQQPPSSVLYVSFGSQSSLDEADFLEIARGLVDSKQSFLWLVRPGFVKGSEWIELLPDGFLGEKGRIVKSAPQQEVLAHKAIGAFWTHGGWNGTLESVCEGVPMIFSPFGLDQPLNARYMSEVLKVGVYLENGFERGEIANAVRRVMVDEEGEVMRQNARVLKDKLDVSLAKGGSSYESLESLVSYISSL

SEQ ID NO: 20 - protein, 457 aa, synthetic construct
MPNTNTTTRRRRRVIMFPVPFPGHLNPMLQLANVLYSRGFSITILHTNFNAPKTSNYPHFTFRFILDNDPQDEWLRNLPTTGPGVGARIPVINKHGADEFRDELELLMASSPEDEEVSCLITDALWYFAQSVADSLNLPRLVLQTGSLFNFHCLVSLPLFLELGYLDPEDKHRLEEPVVGFPMLKVKDIRRAYSHKQESKPILGKMVKQTRASSGVIWNSFKELEESELETIQREIPAPSFLIPLPKHFTASSTSLLDTDRSFFQWLDQQPPNSVLYVSFGSQSSLDEKDFLEIARGLVDSKQSFLWLVRPGFVKGSEWIELLPDGFLGEKGRIVKSAPQQEVLAHEAIGAFWTHGGWNGTLESVCEGVPMIFSPFGLDQPLNARYMSDVLKVGVYLENGFERGEIANAIRRVMVDEEGEEIRQNARVLKQKLDVSLMKGGSSYESLESLVSYISSL

SEQ ID NO: 21 - protein, 457 aa, synthetic construct
MPNTNTTTRRRRRVILFPVPFPGHINPMLQLANVLYSRGFSITILHTNFNAPKTSNYPHFTFRFILDNDPQDEWLRNLPTTGPGVGARIPVINKHGADEFRDELEILMRDTPSDEEVSCLITDALWYFAQSVADSLNLPRLVLQTGSLFNFLCLVSLPLFDELGYLDPEDKHRLDEPVVGFPMLKVKDILRAYSHKQESKPILGKMVKQTKASSGVIWNSFKELEESELETIQREIPAPSFLIPLPKHYTASSTSLLDTDRTFFPWLDQQPPSSVLYVSFGSQSSVDEKDFLEIARGLVDSKQSFLWLVRPGFVKGSEWIELLPDGFLGERGRIVKSAPQQEVLAHEAIGAFWTHGGWNGTLESVCEGVPMIFSDFGLDQPLNARYMSEVLKVGVYLENGFERGEIANAIRRVMVDEEGEEMRENARVLKEKLDVSLMKGGSSYESLESLVSYISSL

SEQ ID NO: 22 - protein, 457 aa, synthetic construct
MPNTNTTTRRRRRVILFPVPFPGHLNPMLQLANVLYSKGFSITILHTNFNAPKTSNYPHFTFRFILDNDPQDEWLRNLPTTGPGVGARIPVINKHGADELRDELELLMRDSPSDEPVSCLITDALWYFAQSVADSLNLPRLVLQTGSLFNFHCLASLPLFDELGYLDLEDKHRLEEPVVGFPMLKVKDIRRAYSHKQESKPILGKMVKQTKASSGVIWNSFKELEESELETIQREIPAPSFLIPLPKHFTASSTSLLDTDRSFFPWLDQQPPNSVLYVSFGSQSQVDEKDFLEIAHGLVDSKQSFLWLVRPGFVKGSEWIEPLPDGFLGERGRIVKSAPQQEVLAHEAIGAFWTHGGWNGTLESVCEGVPMIFSPFGLDQPLNARYMSEVLKVGVYLENGWERGEIANAIRRVMVDEEGEEMRENARKLKQKLDVSLMKGGSSYESLESLVSYISSL

SEQ ID NO: 23 - protein, 457 aa, synthetic construct
MPNTNTTTRRRRRVILFPVPFPGHINPMLQLANVLYSKGFSITILHTNFNAPKTSNYPHFTFRFILDNDPQDEWLRNLPTTGPGVGARIPVINKHGADEFRDELEILMASSPSDEPVACLITDALWYFAQSVADSLNLPRLVLQTGSLFNFHCLASLPLFDELGYLDLEDKHRLEEQVVGFPMLKVKDIRKAYSHKQESKPILGKMVKQTKASSGVIWNSFKELEESELETIQREIPAPSFLIPLPKHFTASSTSLLDTDRSFFPWLDQQPPSSVLYVSFGSQSSVDEKDFLEIAHGLVDSKQSFLWLVRPGFVKGSEWLELLPDGFLGERGRIVKSAPQQEVLAHEAIGAFWTHGGWNGTLESVCEGVPMICSDFGLDQPLNARYMSDVLKVGVYLENGFERGEIANAIRRVMVDEEGEEMRENARVLKDKLDVSLMKGGSSYESLESLVSYISSL

SEQ ID NO: 24 - protein, 457 aa, synthetic construct
MPNTNTTTRRRRRVILFPVPFPGHINPMLQLANVLYSKGFSITILHTNFNAPKTSNYPHFTFRFILDNDPQDEWLRNLPTTGPGVGARIPVINQHGADELRDELELLMRSSPEDEEVSCLITDALWYFAQSVADSLNLPRLVLQTGSLFNFHCLASLPLFDELGYLDLDDKHRLEEQVVGFPMLKVKDILKAYSHKQESKPILGKMVKQTKASSGVIWNSFKELEESELETIQRDIPAPSFLIPLPKHFTASSTSLLDTDRTFFQWLDQQPPSSVLYVSFGSQSQVDEKDFLEIAHGLVDSKQSFLWLVRPGFVKGSEWLELLPDGFLGERGRIVKSAPQQEVLAHEAIGAFWTHGGWNGTLESVCEGVPMICSPFGLDQPLNARYMSDVLKVGVYLENGWERGEIANAIRRVMVDEEGEEMRENARVLKQKLDVSLMKGGSSYESLESLVSYISSL

SEQ ID NO: 25 - protein, 457 aa, synthetic construct
MPNTNTTVRRRRRIILFPVPFPGHINPMLQLANLLYSKGFSITILHTNFNAPKTSNYPHFTFRFILDNDPQDEWLRNLPTTGPGVGARIPVINQHGADELRDELELLMADSPEDEPVSCLITDALWYFTQSVADSLNLPRLVLQTGSLFNFHCLASLPLFDDLGYLDLEDKHRLEEQVVGFPMLKVKDILKAYSHKDESKPILGKMVKQTKASSGVIWNSFKELEESELETIQREIPAPSFLIPLPKHFTASSTSLLDTDRTFFPWLDQQPPNSVLYVSFGSQSQVDEKDFLEIAHGLVDSKQSFLWLVRPGFVKGSEWLEPLPDGFLGERGRIVKSAPQQEVLAHEAIGAFWTHGGWNGTLESVCEGVPMICSPFGLDQPLNARYMSDVLKVGVYLENGWEREEIANAIRRVMVDEEGEEIRENARVLKQKLDVSLMKGGSSYESLESLVSYISSL

SEQ ID NO: 26 - protein, 457 aa, synthetic construct
MPNTSTTVRRRRRVILFPVPFPGHLNPILQLANVLYSKGFSITIFHTNFNKPKTSNYPHFTFRFILDNDPQDEWLRNLPTHGPGAGARIPIINKHGADEFRKELELAMLASPSDEEVACLITDALWYFAQSVADSLNLRRLVLMTSSLFNFHALVSLPQFDELGYLDPEDKTRLEEPASGFPMLKVKDIRSAYSHWQEAKPILDKMIKETRASSGVIWNSFKELEESELETVIREIPAPSFLLPLPKHYTASSSSLLDEDRSTFQWLDQQPPSSVLYVGFGSQSEVDPKDFLEIARGLVDSKQSFLWLVRPGFVKGSEWIEPLPDGFLGEKGRIVKSCPQQEVLAHGAIGAFWTHGGWNGTLESVCEGVPMIFSDFGLDQPLNARYMSDVLKVGVYLENGWERGEIANAIRRVMVDEEGEYMRQNARVLKDKADVSIMKGGSSYESLESLVSYISSL

SEQ ID NO: 27 - protein, 457 aa, synthetic construct
MPNTSTTVVRRRRVILFPVPFPGHLNPILQLANVLYSKGFSITIFHTNFNKPKTSNYPHFTFRFILDNDPQTEWLRNLPTHGPGAGARIPIINKHGADEFRKELEEAMRASEEDEEVACLITDALWYFAQPVADSLNLRRLVLMTSSLFNFHALVSLPQFDELGYLDPEDKTRLEEPASGFPMLKVKDIKSAYSHWQEAKPILDKMIKQTRASSGVIWNSFKELEESELETVIREIPAPSFLIPLPKHLTASSSSLLDEDRTTFQWLDQQPPSSVLYVSFGSQSEVDPKDFLEIARGLVDSKQSFLWLVRPGFVKGYEWVEPLPDGFLGERGRIVKSCPQQEVLAHGAIGAFWTHGGWNGTMESVCEGVPMIFSDFGLDQPLNARYMSDVLHVGVYLENGWERGEIANAIRRVMVDPEGEYMRQNARVLKDKADRSIMKGGSSYESLESLVSYISSL

SEQ ID NO: 28 - protein, 457 aa, synthetic construct
MPNTSTTVRRRRRIILFPVPFPGHLNPILQLANVLYSKGFSITIFHTNFNKPKTSNYPHFTFRFILDNDPQDEWLRNLPTHGPGAGARIPIINKHGADEFRKELELAMLASPEDEEVACLITDALWYFAQSVADSLNLRRLVLMTSSLFNFHALVSLPQFDELGYLDPDDKHRLEEPASGFPMLKVKDIKSAYSHWQEAKPILEKMIKQTRASSGVIWNSFKELEESELETVIREIPAPSFLIPLPKHYTASSSSLLDEDRTTFQWLDQQPPSSVLYVSFGSQSEVDPKDFLEIARGLVDSKQSFLWLVRPGFVKGYTWIEPLPDGFLGERGRIVKSCPQQEVLAHGAIGAFWTHGGWNGTLESVCEGVPMIFSDFGLDQPLNARYMSDVLKVGVYLENGWERGEIANAIRRVMVDEEGEYMRQNARVLKQKADVSIMKGGSSYESLESLVSYISSL

SEQ ID NO: 29 - protein, 457 aa, synthetic construct
MPNTSTTVRRRRRVILFPVPFPGHLNPILQLANVLYRKGFSITIFHTNFNAPKTSNYPHFTFRFILDNDPQDEWLRNLPTHGPGAGARIPIINKHGADEFRRELESLMLASEEDEEVACLITDALWYFAQPVADSLNLRRLVLMTSSLFNFHALVSLPQFDELGYLDPEDKHRLEEPASGFPMLKVKDIKSAYSHWQEAKPILGKMIKQTRASSGVIWNSFKELEESELETVIREIPAPSFLLPLPKHLTASSSSLLDEDRTVFQWLDQQPPSSVLYVSFGSQSEVDEKDFLEIARGLVDSKQSFLWLVRPGFVKGSTWVEPLPDGFLGEKGRIVKSCPQQEVLAHGAIGAFWTHGGWNGTLESVCEGVPMIFSDFGLDQPLNARYMSDVLHVGVYLENGWERGEIANAIRRVMVDEEGEYIRQNARVLKQKADVSLMKGGSSYESLESLVSYISSL

SEQ ID NO: 30 - protein, 457 aa, synthetic construct
MPNTSTTVRRRRRIILFPVPFPGHLNPILQLANVLYSKGFSITIFHTNFNKPKTSNYPHFTFRFILDNDPQDEWLRNLPTHGPGAGARIPIINEHGADELRRELELLMLASEEDEEVSCLITDALWYFAQSVADSLNLRRLVLMTSSLFNFHALVSLPQFDELGYLDPDDKHRLEEPASGFPMLKVKDIKSAYSHWQEAKPILGKMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGYTWVEPLPDGFLGEKGRIVKSCPQQEVLAHGAIGAFWTHGGWNGTLESVCEGVPMIFSDFGLDQPLNARYMSDVLKVGVYLENGWERGEIANAIRRVMVDEEGEYIRQNARVLKQKLDVSLMKGGSSYESLESLVSYISSL

SEQ ID NO: 31 - protein, 457 aa, synthetic construct
MPNTSTTTRRRRRVILFPVPFPGHVNPMLQLANVLYSKGFSITIFHTNFNKPKTSNYPHFTFRFILDNDPQDEWLRNLPTHGPGAGARIPIINEHGADELRRELELLMLASEEDEEVSCLITDALWYFAQSVADSLNLRRLVLMTSSLFNFHALVSLPQFDELGYLDPDDKTRLEEQASGFPMLKVKDIKRAYSHWQEAKPILEKMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWVEPLPDGFLGEKGRIVKSCPQQEVLAHGAIGAFWTHGGWNGTLESVCEGVPMIFSDFGLDQPLNARYMSDVLKVGVYLENGWERGEIANAIRRVMVDEEGEYIRQNARVLKQKLDVSLMKGGSSYESLESLVSYISSL

SEQ ID NO: 32 - protein, 457 aa, synthetic construct
MPNTSTTVRRRRRIILFPVPFPGHINPILQLANVLYSKGFSITIFHTNFNKPKTSNYPHFTFRFILDNDPQDEWLRNLPTHGPGAGARIPIINEHGADELRRELELLMLASEEDGEVSCLITDALWYFAQSVADSLNLRRLVLMTSSLFNFHALVSLPQFDELGYLDPDDKTRLEEQASGFPMLKVKDIKSAYSHWQEAKPILGKMIKQTRASSGVIWNSFKELEESELETVIREIPAPSFLIPLPKHLTASSSSLLDEDRTVFQWLDQQPSSSVLYVSFGSQSVVDEKDFLEIARGLVDSKQSFLWLVRPGFVKGSTWVEPLPDGFLGERGRIVKSCPQQEVLAHGAIGAFWTHGGWNGTLESVCEGVPMIFSDFGLDQPLNARYMSDVLKVGVYLENGWERGEIANAIRRVMVDEEGKYIRQNARVLKQKADVSLMKGGSSYESLESLVSYISSL

SEQ ID NO: 33 - protein, 457 aa, synthetic construct
MPNTSTTVRRRRRIILFPVPFPGHINPILQLANVLYSKGFSITIFHTNFNKPKTSNYPHFTFRFILDNDPQDVWLRNLPSHGPGAGARIPIINEHGADELRRELELLMLASEEDGEVSCLITDALWYFAQSVADSLNLRRLVLMTSSLFNFHALVSLPQFDELGYLDPDDKTRLEEQASGFPMLKVKDIKSAYSHWKEAKPILGNMIKQTKASSGVIWNSFKELEESELETVIREIPAPSFLIPFPKHLTASSSSLLDEDRTVFPWLDQQPSRSVLYVSFGSQSVVDEKDFLEIARGLVDSKQSFLWLVRPGFVKGSTWVEPLPDGFLGERGRIVKSCPQQEVLAHGAIGAFWTHGGWNGTLESVCEGVPMIFSPFGLDQPLNARYMTDVLKVGVYLENGWERGEIANAIRRVMVDEEGEYIRQNASVLKQKADVSLMKGGSSYESLESLVSYISSL

SEQ ID NO: 34 - protein, 457 aa, synthetic construct
MPNTSTTVRRRRRIILFPVPFPGHINPMLQLANVLYSKGFSITIFHTNFNKPKTSNYPHFTFRFILDNDPQDEWLRNLPTHGPGAGARIPIINEHGADELRRELELLMLDSEEDEEVSCLITDALWYFAQSVADSLNLRRLVLMTSSLFNFHALVSLPQFDELGYLDPDDKHRLEEQASGFPMLKVKDIKSAYSHWQEAKPILDKMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWIELLPDGFLGEKGRIVKSCPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 35 - protein, 457 aa, synthetic construct
MPNTSPTVRRRRRIILFPVPFPGHINPMLQLANVLYSKGFSITIFHTNFNKPKTSNYPHFTFRFILDNDPQDEWLRNLPTHGPGAGARIPIINEHGADELRRELELLMLASEEDEEVSCLITDALWYFAQSVADSLNLRRLVLMTSSLFNFHALVSLPQFDELGYLDPDDKHRLEEQASGFPMLKVKDIKSAYSHWQEAKPILDKMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWVELLPDGFLGEKGRIVKSCPQQEVLAHKAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 36 - protein, 457 aa, synthetic construct
MPNTSTTVRRRRRIILFPVPFPGHINPMLQLANVLYSKGFSITIFHTNFNKPKTSNYPHFTFRFILDNDPQDEWLRNLPTHGPGAGARIPIINEHGADELRRELELLMLASEEDEEVSCLITDALWYFAQSVADSLNLRRLVLMTSSLFNFHALVSLPQFDELGYLDPDDKHRLEEQASGFPMLKVKDIKSAYSHWQEAKPILGKMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWVEPLPDGFLGERGRIVKSCPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 37 - protein, 457 aa, synthetic construct
MPNTSTTVRRRRRIILFPVPFPGHINPMLQLANVLYSKGFSITIFHTNFNKPKTSNYPHFTFRFILDNDPQDEWLRNLPTHGPGAGARIPIINEHGADELRRELELLMLASEEDEEVSCLITDALWYFAQSVADSLNLRRLVLMTSSLFNFHALVSLPQFDELGYLDPDDKHRLEEQASGFPMLKVKDIKSAYSHWQEAKPILDKMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWVEPLPDGFLGERGRIVKSCPQQEVLAHKAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 38 - protein, 457 aa, synthetic construct
MPNTSTTVRRRRRIILFPVPFPGHINPMLQLANVLYSKGFSITIFHTNFNKPKTSNYPHFTFRFILDNDPQDEWLRNLPTHGPGAGARIPIINEHGADELRRELELLMLASEEDEEVSCLITDALWYFAQSVADSLNLRRLVLMTSSLFNFHALVSLPQFDELGYLDPDDKHRLEEQASGFPMLKVKDIKSAYSHWQEAKPILGNMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWVEPLPDGFLGERGRIVKSCPQQEVLAHKAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 39 - protein, 457 aa, synthetic construct
MPNTSTTVRRRRRIILFPVPFPGHINPILQLANVLYSKGFSITIFHTNFNKPKTSNYPHFTFRFILDNDPQDVWLRNLPTHGPGAGARIPIINEHGADELRRELELLMLASEEDEEVSCLITDALWYFAQSVADSLNLRRLVLMTSSLFNFHALVSLPQFDELGYLDPDDKHRLEEQASGFPMLKVKDIKSAYSHWQEAKPILGNMIKQTRASSGVIWNSFKELEESELETVIREIPAPSFLIPLPKHLTASSSSLLDEDRTVFQWLDQQPPSSVLYVSFGSQSEVDEKDFLEIARGLVDSKQSFLWLVRPGFVKGSTWVEPLPDGFLGERGRIVKSCPQQEVLAHGAIGAFWTHGGWNGTLESVCEGVPMIFSDFGLDQPLNARYMSDVLKVGVYLENGWERREIANAIRRVMVDEEGEYIRQNARVLKQKADVSLMKGGSSYESVESLVSYVSSL

SEQ ID NO: 40 - protein, 457 aa, synthetic construct
MPNTSTTVRRRRRIILFPVPFPGHINPILQLANVLYSKGFSITIFHTNFNKPKTSNYPHFTFRFILDNDPQDVWLRNLPTHGPGAGARIPIINEHGADELQRELELMMLASEEDEEVSCLITDALWYFAQSVADSLNLRPLVLMTSSLFNFHALVSLPQFDELGYLDPDDKHRLEEQASGFPMLKVKDIKSAYSHWQEAKPILGNMTKQTKASSGVIWNSFKELEESELETVIREIPAPSFLIPLPKHLTASSSSGLDEDRTVFPWLDQQPSRSVLYVSFGSQSVVDEKDFLEIARGLVDSKQSFLWLVRPGFVKGSTWVEPLPDGFVGERGRIVKSCPQQEVLAHGAIGAFWTHGGWNGTLESVCEGVPMIFSDFGLDQPLNARYMSDVLKVGVYLENGWERREIANAIRRVMVDEEGEYIRQNARVLKQKADVSLMKGGSSYESVESLVSYVSSL

SEQ ID NO: 41 - protein, 457 aa, synthetic construct
MPNTSTTTRRRRRVILFPVPFPGHINPMLQLANVLYSKGFSITILHTNFNAPKTSNYPHFTFRFILDNDPQDEWLRNLPTHGPGAGARIPLINKHGADEFRDELESLMADSPSDEEVSCLITDALWYFAQSVADSLNLPRLVLMTSSLFNFHALVSLPLFLELGYLDPEDKHRLEEPVVGFPMLKVKDIRRAYSHKQEAKPILDKMIEQTKASSGVIWNSFKELEESELETVRREIPAPSFLIPLPKHFTASSSSLLDEDRSFFQWLDQQPPSSVLYVSFGSQSEVDEKDFLEIARGLVDSKQSFLWLVRPGFVKGSEWIELLPDGFLGERGRIVKSCPQQEVLAHEAIGAFWTHGGWNGTLESVCEGVPMIFSPFGLDQPLNARYMSDVLKVGVYLENGFERGEIANAIRRVMVDEEGEEMRQNARVLKDKADRSLMKGGSSYESLESLVSYISSL

SEQ ID NO: 42 - protein, 457 aa, synthetic construct
MPNTSTTTRRRRRVILFPVPFPGHINPMLQLANVLYSKGFSITILHTNFNAPKTSNYPHFTFRFILDNDPQDEWLRNLPTHGPGAGARIPVINKHGADEFRQELESLMADSPSDEEVSCLITDALWYFAQSVADSLNLPRLVLMTSSLFNFHALVSLPLFLELGYLDPEDKHRLEEPVVGFPMLKVKDIRRAYSHWQEAKPILDKMIEQTKASSGVIWNSFKELEESELETVRREIPAPSFLIPLPKHFTASSSSLLDEDRTFFQWLDQQPPSSVLYVSFGSQSEVDEKDFLEIARGLVDSKQSFLWLVRPGFVKGSEWIELLPDGFLGERGRIVKSCPQQEVLAHEAIGAFWTHGGWNGTLESVCEGVPMIFSPFGLDQPLNARYMSDVLKVGVYLENGFERGEIANAIRRVMVDEEGEEMRQNARKLKQKADRSLMKGGSSYESLESLVSYISSL

SEQ ID NO: 43 - protein, 457 aa, synthetic construct
MPNTSTTTRRRRRIILFPVPFPGHINPMLQLANVLYSKGFSITILHTNFNAPKTSNYPHFTFRFILDNDPQDEWLRNLPTHGPGAGARIPLINKHGADEFRDELELLMRDSPSDEEVACLITDALWYFAQSVADSLNLPRLVLMTSSLFNFHALVSLPLFDELGYLDPEDKHRLEEPVVEFPMLKVKDILRAYSHWQEAKPILDKMIKQTKASSGVIWNSFKELEESELETVRREIPAPSFLIPLPKHFTASSSSLLDEDRSFFPWLDQQPPSSVLYVSFGSQSEVDEKDFLEIARGLVDSKQSFLWLVRPGFVKGSEWLEPLPDGFLGERGRIVKSCPQQEVLAHEAIGAFWTHGGWNGTLESVCEGVPMICSPFGLDQPLNARYMSDVLKVGVYLENGFERGEIANAIRRVMVDEEGEEIRENARVLKDKADVSLMKGGSSYESLESLVSYISSL

SEQ ID NO: 44 - protein, 457 aa, synthetic construct
MPNTSTTTRRRRRIILFPVPFPGHINPMLQLANVLYSKGFSITILHTNFNAPKTSNYPHFTFRFILDNDPQDEWLRNLPTHGPGAGARIPLINEHGADEFRDELELLMADSESDEEVACLITDALWYFAQSVADSLNLPRLVLMTSSLFNFHALVSLPLFDELGYLDPEDKHRLEEPVVGFPMLKVKDIKRAYSHWQEAKPILDKMIKQTKASSGVIWNSFKELEESELETVRREIPAPSFLGPLPKHFTASSSSLLDEDRSFFPWLDQQPPSSVLYVSFGSQSEVDEKDFLEIARGLVDSKQSFLWLVRPGFVKGSEWLELLPDGFLGERGRIVKSCPQQEVLAHGAIGAFWTHGGWNGTMESVCEGVPMICSPFGLDQPLNARYMSDVLKVGVYLENGFEREEIANAIRRVMVDEEGEEIRQNARVLKDKADVSLMKGGSSYESLESLVSYISSL

SEQ ID NO: 45 - protein, 457 aa, synthetic construct
MPNTSTTTRRRRRVILFPVPFPGHINPMLQLANVLYSKGFSITILHTNFNAPKTSNYPHFTFRFILDNDPQDEWLRNLPTHGPGAGARIPLINKHGADELRDELELLMASSEEDEPVSCLITDALWYFAQSVADSLNLPRLVLMTSSLFNFHALVSLPLFDELGYLDLDDKHRLEEQVVGFPMLKVKDILKAYSHWQEAKPILGKMIKQTKASSGIIWNSFKELEESELETVRREIPAPSFLIPLPKHFTASSSSLLDEDRTFFPWLDQQPPSSVLYVSFGSQSEVDEKDFLEIAHGLVDSKQSFLWLVRPGFVKGSEWIELLPDGFLGERGRIVKSCPQQEVLAHEAIGAFWTHGGWNGTLESVCEGVPMICSPFGLDQPLNARYMSDVLKVGVYLENGFEREEIANAIRRVMVDEEGEEMRQRARVLKQKLDVSIMKGGSSYESLESLVSYISSL

SEQ ID NO: 46 - protein, 457 aa, synthetic construct
MPNTSTTTRRRRRIILFPVPFPGHINPMLQLANVLYSRGFSITILHTNFNAPKTSNYPHFTFRFILDNDPQTEWLRNLPTHGPGAGARIPLINKHGADEFRDELELLMRSSPEDEPVSCLITDALWYFAQSVADSLNLPRLVLMTSSLFNFHALVSLPLFDELGYLDLDDKHRLEEQVVGFPMLKVKDILKAYSHKQEAKPILTKMIKQTKASSGIIWNSFKELEESELETVRREIPAPSFLIPLPKHFTASSSSLLDEDRTFFPWLDQQPPKSVLYVSFGSQSEVDEKDFLEIAHGLVDSKQSFLWLVRPGFVKGSEWLELLPDGFLGERGRIVKSCPQQEVLAHEAIGAFWTHGGWNGTLESVCEGVPMICSPFGLDQPLNARYMSDVLKVGVYLENGFEREEIANAIRRVMVDEEGEEIRQNARVLKQKLDVSLMKGGSSYESLESLVSYISSL

SEQ ID NO: 47 - protein, 457 aa, synthetic construct
MPNTSTTVRRRRRVILFPVPFPGHINPMLQLANVLYSKGFSITILHTNFNAPKTSNYPHFTFRFILDNDPQDEWLRNLPTHGPGAGARIPLINEHGADELRDELELLLASSEEDEPVSCLITDALWYFAQSVADSLNLPRLVLMTSSLFNFLALASLPLFDELGYLDLDDKHRLEEQVVGFPMLKVKDILKAYSHKQEAKPILGKMIKQTKASSGIIWNSFKELEESELETVRRDIPAPSFLIPLPKHFTASSSSLLDEDRSFFPWLDQQPPSSVLYVSFGSQSQVDEKDFLEIAHGLVDSKQSFLWLVRPGFVKGSEWIEPLPDGFLGERGRIVKSCPQQEVLAHEAIGAFWTHGGWNGTLESVCEGVPMICSPFGLDQPLNARYMSDVLKVGVYLENGWEREEIANAIRRVMVDEEGEEIRQNARVLKQKLDVSLMKGGSSYESLESLVSYISSL

SEQ ID NO: 48 - protein, 457 aa, synthetic construct
MPNTSTTVRRRRRVILFPVPFPGHINPMLQLANLLYSKGFSITILHTNFNAPKTSNYPHFTFRFILDNDPQDEWLRNLPTHGPGAGARIPLFNQHGADELRHELELLLASSESDEPVSCLITDALWYFTQSVADSLNLPRLVLMTSSLFNFLALASLPLFDDLGYLNLDDKHRLEEQVVEFPMLKVKDILKAYSHKDEAKPILDKMVKQTKASSGIIWNSFKELEESELETVRRDIPAPSFLIPFPKHFTASSSSLLDEDRTFFPWLDQQPPNSVLYVSFGSQSQVDEKDFLEIAHGLVDSKQSFLWLVRPGFVKGSEWIEPLPDGFLGERGRIVKSCPQQEVLAHEAIGAFWTHGGWNGTLESVCEGVPMICSPFGLDQPLNARYMSDVLKVGVYLENGWEREEIANAIRRVMVDEEGEEIRENARVLKQKLDVSLMKGGSSYESLESLVSYISSL

SEQ ID NO: 49 - protein, 457 aa, synthetic construct
MPNTNMQTRRGRRLVLFPVPFPGHLNPMLQLANILHSKGFSITIIHTNFNSPNTSNYPHFTFQPIPDGLLESEWLRNLPAETPGVGARIPVHNKTCVDPFRKCMERLMSDVLSEEPISCLITDALWYFTQAVADSLKLPRIVLRTGSLSSFHAFVSFPLLLEKGYLPKQDSHQLAEPVVEFPHLKVKDIRVANSHIPESKEILESMIKETKASSGVIWNSFTELEQPALITIQQDFPIPVFILPFHKHYPASSSSLLTHDQSSISWLDQQKPLSVLYVGFGSLAAMDEKQFLEMAWGLANSKQKFLWVVRPGFIRGSEWLEPLPDGFLEASGRIVKSAPQQQVLAHPAIGAFWTHGGWNGTLESICEGVPMICSDFGTDQMVNARYVSHVWKVGVQLENGWERGEIEKAIRRLMVDKEGEEMRQRISLLKEKVNLCLEQGGSSYKSLESLISYISSL

SEQ ID NO: 50 - protein, 457 aa, synthetic construct
MPNTNTTVRRRRRVIMFPVPFPGHLNPMLQLANVLYFKGFSITIFHTNFNAPKTSNYPHFTFRFILDNDPQDEWLRNLPTHGPGVGARIPVINKHGADELRRELEICMAASEEDEEVSCLITDALWYFAQSVADSLNLRRLVLRTGSLFNFHALVSLPKFDELGYLDPDDKTRLEEQAAGFPMLKVKDIRSAYSHIQQSKEILGKMTKQTRASSGVIWNSFKELEESELETIQREIPAPSFLLPLPKHYTASSSSLLDHDRSVFQWLDQQPPSSVLYVGFGSTSEVDPKDFLEIARGLVASKQSFLWVVRPGFVKGYTWVEPLPDGFLGERGRIVKSAPQQEVLAHGAIGAFWTHGGWNGTLESVCEGVPMIFSDFGLDQPLNARYMSDVLHVGVYLENGWERGEIANAIRRVMVDEEGEVMRQNARVLKQKADVSLMPGGSSYESLESLVSYISSL

SEQ ID NO: 51 - protein, 457 aa, synthetic construct
MPNTNTTVRRRRRIILFPVPFPGHLNPMLQLANVLYSKGFSITIFHTNFNKPKTSNYPHFTFRFILDNDPQDVWLRNLPTHGPGVGARIPVINKHGADELRRELELLMLASEEDEEVSCLITDALWYFAQSVADSLNLRRLVLQTGSLFNFHALVSLPQFDELGYLDPDDKKRLEEQVSGFPMLKVKDIRCAYSHIQISKEILGKMIKQTKASSGVIWNSFKELEESELETVIREIPAPSFLLPLPKHYTASSSSLLDHDRTVFQWLDQQPPSSVLYVGFGSTSEVDAADFLEIARGLVDSKQSFLWVVRPGFVKGSTWVEPLPDGFLGERGRIVKSAPQQEVLAHGAIGAFWTHGGWNGTLESVCEGVPMICSDFGLDQPLNARYMSDVLGVGVYLENGWERGEIANAIRRVMVDPEGEYIRQNARKLKQKADVSLMKGGSSYESLESLVSYISSL

SEQ ID NO: 52 - protein, 457 aa, synthetic construct
MPNTNTTVRRRRRIILFPVPFPGHLNPMLQLANVLYSLGFSITIFHTNFNAPKTSNYPHFTFRFILDNDPQDEWLRNLPTTGPGVGARIPVINKHGADELRTELELLMLASEEDEEVACLITDALWYFAQSVADSLNLRRLVLRTGSLFNFHALVSLPQFDELGYLDPDDKTRLEEQASGFPMLKVKDIRSAYSHIQKSKEILEKMIKQTRASSGVIWNSFKELEESELETVIREIPAPSFLLPLPKHYTASSSSLLDHDPTTFQWLDQQPPSSVLYVGFGSTSELDEKDFLEIARGLVDSKQSFLWVVRPGFVKGSTWVEPLPDGFLGERGRIVKSAPQQEVLAHTAIGAFWTHGGWNGTLESVCEGVPMIFSDFGLDQPLNARYMSDVLKVGVYLENGWERGEIANAIRRVMVDEEGEYMRQNARVLKQKADVSLMKGGSSYESLESLVAYISSL

SEQ ID NO: 53 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADELRRELELLMRESEQDEEVSCLITDALWYFAQSVADSLNLPRLVLMTGSLFNFHALVSLPQFDELGYLDPDNKTRLEEQASGFPMLKVKDIRSATSHIQDSKEILGKMIKQTRASSGVIWNSFKELEESELETVIREIPAPSFLLPLPKHYTASSSSLLDHDRTVFQWLDQQPPRSVLYVGFGSTSEVDEKDFLEIARGLVDSKQSFLWVVRPGFVKGSTWVEPLPDGFHGEKGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 54 - protein, 457 aa, synthetic construct
MPNTNTTVVRRRRVIMFPVPFPGHLNPMLQLANVLYSKGFSITIFHTNFNKPKTSNYPHFTFRFILDNDPQDEWLRNLPTHGPGVGARIPVINKHGADELRKELEICMVASEEDEEVACLITDALWYFAQSVADSLNLKRLVLMTGSLFNFHALVSLPQFDELGYLDPEDKTRLDEPASGFPMLKVKDIRCAYSHIQISKEILEKMVKQTRASSGVIWNSFKELEESELETIIREIPAPSFLLPLPKHYTASSSSLLDHDRSTFQWLDQQPPSSVLYVGFGSQSELDPKDFLEIARGLVDSKQSFLWVVRPGFVKGSTWIEPLPDGFLGERGRIVKSAPQQEVLAHKAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLERLVSYISSL

SEQ ID NO: 55 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINEHGADEFRKELELLMLASPSDEEVSCVITDALWYFAQSVADSLNLRRLVLQTGSLFNFHCLVSLPKFDELGYLDPDDKHRLEEPAVGFPMLKVKDIKSAYSHWQESKPILGKMVKQTRASSGVIWNSFKELEESELETVWREIPAPSFLIPLPKHYRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 56 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINEHGADEFRKELELLMLASPSDEEVSCVITDALWYFAQSVADSLNLRRLVLQTGSLFNFHCLVSLPKFDELGYLDPDDKHRLEEPAVGFPMLKVKDIKSAYSHWQESKPILEKMVKQTRASSGVIWNSFKELEESELETVWREIPAPSFLIPLPKHYRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 57 - protein, 457 aa, synthetic construct
MPNTNTTVVRRRRIILFPVPFPGHWNPMLQLANVLYSKGFSITIFHTNFNKPKTSNYPHFTFRFILDNDPQDEWLRNLPTHGPGVGARIPIINKHGADEFRKELELLMLASEEDEEVSCLITDALWYFAQSVADSLNLKRLVLQTGSLFNFHCLVSLPKFDELGYLDPDDKTRLEEPASGFPMLKVKDIKSAYSHWQESKPILDKMVKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLERLVSYISSL

SEQ ID NO: 58 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPQINEHGADEFRRELELLMLASESDEEVACLITDALWYFAQSVADSLNLRRLVLQTGSLFNFHCLVSLPKFDELGYLDPDDKTRLEEPAVGFPMLKVKDIKSAYSHWQESKPILEKMVKQTRASSGVIWNSFKELEESELETVWREIPAPSFLIPLPKHYRASSTSLLDTDRTVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADVSIMKGGSSYESLESLVSYISSL

SEQ ID NO: 59 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELELLMLASGSDEEVACLITDALWYFAQSVADSLNLPRLVLQTGSLFNFHCLVSLPKFDELGYLDPDDKTRLEEPAVGFPMLKVKDIKSAYSHWQESKPILEKMIKQTRASSGVIWNSFKELEESELETVIREIPAPSFLIPLPKHLRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLRVGVYLENGWERGEIANAIRRVMVDEEGEYMRQNARVLKQKADRSIMKGGSSYESLESLVSYISSL

SEQ ID NO: 60 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVSCLITDALWYFAQSVADSLNLKRLVLQTGSLFNFHCLVSLPKFDELGYLDPDDKTRLEEPAVGFPMLKVKDIKSAYSHWQESKPILGKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSEWIEPLPDGFLGEKGRVVKSAPQQEVLAHGAIGAFWTHGGWNGTMESVCEGVPMIFSDFGLDQPLNARYMSEVLRVGVYLENGWERGEIANAIRRVMVDEEGEYMRQNARVLKQKADRSIMKGGSSYESLESLVSYISSL

SEQ ID NO: 61 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELESAMLASESDEEVACLITDALWYFAQSVADSLNLPRLVLQTGSLFNFHCLVSLPKFDELGYLDPDDKTRLEEPAVGFPMLKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTLESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 62 - protein, 457 aa, synthetic construct
MPNTNTTVRRRRRIILFPVPFPGHINPMLQLANVLYSKGFSITIFHTNFNAPKTSNYPHFTFRFILDNDPQDEWLRNLPTHGPGVGARIPVINKHGADEFRRELELLMLASESDEEVSCLITDALWYFAQSVADSLNLRRLVLQTGSLFNFHCLVSLPKFDELGYLDPDDKTRLEEPAVGFPMLKVKDIKSAYSHWQESKPILGKMIKQTRASSGVIWNSFKELEESELETVIREIPAPSFLIPLPKHYRASSTSLLDTDRSVFQWLDQQPPSSVLYVGFGSQSEVDPKDFLEIARGLVDSKQSFLWLVRPGFVKGSTWVEPLPDGFLGEKGRVVKSAPQQEVLAHGAIGAFWTHGGWNGTMESVCEGVPMIFSDFGLDQPLNARYMSDVLRVGVYLENGWERGEIANAIRRVMVDEEGEYMRQNARVLKQKADRSIMKGGSSYESLESLVSYISSL

SEQ ID NO: 63 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRRELESLMLASPSDEEVSCLITDALWYFAQSVADSLNLRRLVLQTGSLFNFHCLVSLPKFDELGYLDPDDKTRLEEPAVGFPMLKVKDIKSAYSHWQESKPILDKMVKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEYMRQNARVLKQKADRSIMKGGSSYESLERLVSYISSL

SEQ ID NO: 64 - protein, 457 aa, synthetic construct
MPNTNTTVRRRRRIILFPVPFPGHVNPMLQLANVLYSKGFSITIFHTNFNKPKTSNYPHFTFRFILDNDPQDEWLRNLPTHGPGVGARIPIINKHGADEFRKELELLMLASPSDEEVACLITDALWYFAQSVADSLNLKRLVLQTGSLFNFHCLVSLPKFDELGYLDPDDKTRLEEPAVGFPMLKVKDIKSAYSHWQESKPILEKMIKQTRASSGVIWNSFKELEESELETVIREIPAPSFLIPLPKHLRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLKVGVYLENGWERGEIANAIRRVMVDEEGEYMRQNARVLKQKADRSIMKGGSSYESLESLVSYISSL

SEQ ID NO: 65 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVSCLITDALWYFAQSVADSLNLRRLVLQTGSLFNFHCLVSLPKFDELGYLDPDDKTRLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEYMRQNARVLKQKADRSIMKGGSSYESLESLVSYISSL

SEQ ID NO: 66 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVACLITDALWYFAQSVADSLNLKRLVLQTGSLFNFHCLVSLPKFDELGYLDPEDKTRLEEPAVGFPMLKVKDIKSAYSHWQESKPILEKMIKQTRASSGVIWNSFKELEESELETVWREIPAPSFLIPLPKHYRASSTSLLDTDRSVFQWLDQQPPSSVLYVGFGSQSEVDPKDFLEIARGLVDSKQSFLWLVRPGFVKGYTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 67 - protein, 457 aa, synthetic construct
MPNTNTTVRRRRRIILFPVPFPGHVNPMLQLANVLYSKGFSITIFHTNFNKPKTSNYPHFTFRFILDNDPQDEWLRNLPTHGPGVGARIPIINKHGADEFRRELELLMLASASDEEVSCLITDALWYFAQSVADSLNLKRLVLQTGSLFNFHCLVSLPKFDELGYLDPDDKTRLEEPASGFPMLKVKDIKSAYSHWQESKPILEKMVKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 68 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINEHGADEFRKELELLMLASESDEEVSCLITDALWYFAQSVADSLNLKRLVLQTGSLFNFHCLVSLPKFDELGYLDPDDKTRLEEPAVGFPMLKVKDIKSAYSHWQESKPILGKMVKQTRASSGVIWNSFKELEESELETVIREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTLESVCEGVPMIFSDFGLDQPLNARYMSDVLHVGVYLENGWERGEIANAVRRVMVDEEGEYMRQNARVLKQKADRSIMKGGSSYESLESLVSYISSL

SEQ ID NO: 69 - protein, 457 aa, synthetic construct
MPNTNTTVVRRRRIILFPVPFPGHINPMLQLANVLYSKGFSITIFHTNFNKPKTSNYPHFTFRFILDNDPQDEWLRNLPTHGPGVGARIPIINKHGADEFRKELELLMLASESDEEVSCLITDALWYFAQSVADSLNLRRLVLQTGSLFNFHCLVSLPKFDELGYLDPDDKHRLEEPAVGFPMLKVKDIKSAYSHWQESKPILDKMIKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 70 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVSCLITDALWYFAQSVADSLNLKRLVLQTGSLFNFHCLVSLPKFDELGYLDPDDKTRLDEPAVGFPMLKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMEAVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 71 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELELLMLASKSDEEVACLITDALWYFAQSVADSLNLPRLVLQTGSLFNFHCLVSLPKFDELGYLDPDDKTRLEEPAVGFPMLKVKDIKSAYSHWQESKPILEKMIKQTRASSGVIWNSFKELEESELETVIREIPAPSFLIPLPKHLRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLKVGVYLENGWERGEIANAIRRVMVDEEGEYMRQNARVLKQKADRSIMKGGSSYESLESLVSYISSL

SEQ ID NO: 72 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPIINKHGADEFRRELELLMLASPSDEEVSCLITDALWYFAQSVADSLNLRRLVLQTGSLFNFHCLVSLPKFDELGYLDPDDKTRLEEPAVGFPMLKVKDIKSAYSHWQESKPILDKMVKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEYMRQNARVLKQKADRSIMKGGSSYESLERLVSYISSL

SEQ ID NO: 73 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELESLMLASESDEEVACVITDALWYFAQSVADSLNLRRLVLQTGSLFNFHCLVSLPKFDELGYLDPDDKTRLEEPAVGFPMLKVKDIKSAYSHWQESKPILEKMVKQTRASSGVIWNSFKELEESELETVQREIPAPSFLIPLPKHYTASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 74 - protein, 457 aa, synthetic construct
MPNTNTTVRRRRRIILFPVPFPGHLNPMLQLANVLYSKGFSITIFHTNFNKPKTSLYPHFTFRFILDNDPQDEWLRNLPTHGPGVGARIPVINKHGADEFRKELELLMLASESDEEVSCLITDALWYFAQSVADSLNLKRLVLQTGSLFNFHCLVSLPKFDELGYLDPDDKTRLEEPAVGFPMLKVKDIKSAYSHWQESKPILSKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 75 - protein, 457 aa, synthetic construct
MPNTNTTVVRRRRIILFPVPFPGHLNPMLQLANVLYSKGFSITIFHTNFNKPKTSNYPHFTFRFILDNDPQDEWLRNLPTHGPGVGARIPIINKHGADEFRKELELLMLASESDEEVSCLITDALWYFAQSVADSLNLRRLVLQTGSLFNFHCLVSLPKFDELGYLDPDDKTRLEEPAVGFPMLKVKDIKSAYSHWQESKPILGKMIKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTVESVCEGVPMIFSDFGLDQPLNARYMSDVLRVGVYLENGWERGEIANAIRRVMVDEEGEYMRQNARVLKQKADRSIMKGGSSYESLESLVSYISSL

SEQ ID NO: 76 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRRELELLMLASPSDEEVSCLITDALWYFAQSVADSLNLKRLVLQTGSLFNFHCLVSLPKFDELGYLDPDDKTRLEEPAVGFPMLKVKDIKSAYSHWQESKPILEKMVKQTRASSGVIWNSFKELEESELETVWREIPAPSFLIPLPKHYTASSTSLLDTDRTVFQWLDQQPPSSVLYVGFGSQSEVDPKDFLEIARGLVDSKQSFLWLVRPGFVKGSEWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 77 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADEFRKELELLMLASESDEEVACLITDALWYFAQSVADSLNLRRLVLQTGSLFNFHCLVSLPKFDELGYLDPDDKTRLEEPAVGFPMLKVKDIKSAYSHWQESKPILGKMVKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGYTWIEPLPDGFLGEKGRVVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 78 - protein, 457 aa, synthetic construct
MPNTNPTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVSCLITDALWYFAQSVADSLNLKRLVLQTGSLFNFHCLVSLPKFDELGYLDPDDKTRLDEPAVGFPMLKVKDIKSAYSHWQESKPILDKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 79 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINEHGADEFRRELELLMLASESDEEVSCLITDALWYFAQSVADSLNLPRLVLQTGSLFNFHCLVSLPKFDELGYLDPDDKTRPEEPAVGFPMLKVKDIKSAYSHWQESKPILGKMIKQTRASSGVIWNSFKELEESELETVIREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 80 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVACLITDALWYFAQSVADSLNLKRLVLQTGSLFNFHCLVSLPKFDELGYLDPDDKTRLEEPAVGFPMLKVKDIKSAYSHWQESKPILEKMIKQTRASSGVIWNSFKELEESELETVWREIPAPSFLIPLPKHYRASSTSLLDTDRSVFQWLDQQPPSSVLYVGFGSQSEVDPKDFLEIARGLVDSKQSFLWLVRPGFVKGYTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADGSIMKGGSSYESLESLVSYISSL

SEQ ID NO: 81 - protein, 457 aa, synthetic construct; identical to SEQ ID NO: 55

SEQ ID NO: 82 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPQINKHGADEFRKELELLMLASKSDEEVACLITDALWYFAQSVADSLNLKRLVLQTGSLFNFHCLVSLPKFDELGYLDPDDKTRLEEPASGFPMLKVKDIKSAYSHWQESKPILEKMIKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 83 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFRKELELLMLASESDEEVSCLITDALWYFAQSVADSLNLKRLVLQTGSLFNFHCLVSLPKFDELGYLDPDDKTRLEEPAVGFPMLKVKDIKSAYSHWQESKPILMKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMESVCEGVPMIFSDFGLDQPLNARYMSEVLHVGVYLENGWERGEIANAIRRVMVDEEGEYMRQNARVLKQKADRSIMKGGSSYESLESLVSYISSL

SEQ ID NO: 84 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELELLMLASGSDEEVACLITDALWYFAQSVADSLNLPRLVLQTGSLFNFHCLVSLPKFDELGYLDPDDKTRLEEPAVGFPMLKVKDIKSAYSHWQESKPILEKMIKQTRASSGVIWNSFKELEESELETVIREIPAPSFLIPLPKHLRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLKVGVYLENGWERGEIANAIRRVMVDEEGEYMRQNARVLKQKADRSIMKGGSSYESLESLVSYISSL

SEQ ID NO: 85 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPLINKHGADEFRKELELLMLASESDEEVSCLITDALWYFAQSVADSLNLKRLVLQTGSLFNFHCLVSLPKFDELGYLDPDDKTRLEEPAVGFPMLKVKDIKSAYSHWQESKPILGKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMESVCEGVPMIFSDFGLDQPLNARYMSEVLRVGVYLENGWERGEIANAIRRVMVDEEGEYMRQNARVLKQKADRSIMKGGSSYESLESLVSYISSL

SEQ ID NO: 86 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELESAMLASESDEEVACLITDALWYFAQSVADSLNLRRLVLQTGSLFNFHCLVSLPKFDELGYLDPDDKTRLEEPAVGFPMLKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTLESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 87 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELELLMLASESDEEVSCLITDALWYFAQSVADSLNLRRLVLQTGSLFNFHCLVSLPKFDELGYLDPDDKTRLEEPAVGFPMLKVKDIKSAYSHWQESKPILDKMVKETRASSGVIWNSFKELEESELETVWREIPAPSFLIPLPKHYRASSTSLLDTDRTVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLERLVSYISSL

SEQ ID NO: 88 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINEHGADEFRKELELLMLASEKDEEVSCLITDALWYFAQSVADSLNLKRLVLQTGSLFNFHCLVSLPKFDELGYLDPDDKTRLEEPAVGFPMLKVKDIKSAYSHWQESKPILEKMVKQTRASSGVIWNSFKELEESELETVWREIPAPSFLIPLPKHLRASSTSLLDTDRTVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 89 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMLASESDEEVSCLITDALWYFAQSVADSLNLKRLVLQTGSLFNFHCLVSLPKFDELGYLDPDDKTRLEEPAVGFPMLKVKDIKSAYSHWQESKPILDKMVKQTRASSGVIWNSFKELEESELETVQREIPAPSFLIPLPKHYRASSTSLLDTDRSVFQWLDQQPPSSVLYVGFGSQSEVDPKDFLEIARGLVDSKQSFLWLVRPGFVKGSEWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEVMRQNARVLKQKADRSIMKGGSSYESLESLVSYISSL

SEQ ID NO: 90 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPQINEHGADEFRRELELLMLASESDEEVACLITDALWYFAQSVADSLNLKRLVLQTGSLFNFHCLVSLPKFDELGYLDPDDKTRLEEPAVGFPMLKVKDIKSAYSHWQESKPILGKMVKQTRASSGVIWNSFKELEESELETVIREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRVVKSAPQQEVLAHGAIGAFWTHGGWNGTMESVCEGVPMIFSDFGLDQPLNARYMSDVLRVGVYLENGWERGEIANAIRRVMVDEEGEYMRQNARVLKQKADRSIMKGGSSYESLESLVSYISSL

SEQ ID NO: 91 - protein, 457 aa, synthetic construct
MPNTNTTVVRRRRIILFPVPFPGHINPMLQLANVLYSKGFSITIFHTNFNKPKTSNYPHFTFRFILDNDPQDEWLRNLPTHGPGVGARIPIINKHGADEFRKELELLMLASESDEEVSCLITDALWYFAQSVADSLNLRRLVLQTGSLFNFHCLVSLPKFDELGYLDPDDKHRLEEPAVGFPMLKVKDIKSAYSHWQESKPILEKMIKQTRASSGVIWNSFKELEESELETVIREIPAPSFLIPLPKHYRASSTSLLDTDRTVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 92 - protein, 457 aa, synthetic construct
MPNTNTTVRRRRRIILFPVPFPGHLNPMLQLANVLYSKGFSITIFHTNFNKPKTSLYPHFTFRFILDNDPQDEWLRNLPTHGPGVGARIPVINKHGADEFRKELELLMLASESDEEVACLITDALWYFAQSVADSLNLKRLVLQTGSLFNFHCLVSLPKFDELGYLDPEDKTRLEEPAVGFPMLKVKDIKSAYSHWQESKPILEKMIKQTRASSGVIWNSFKELEESELETVWREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 93 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINEHGADEFRKELELMMLASESDEEVSCVITDALWYFAQSVADSLNLKRLVLQTGSLFNFHCLVSLPKFDELGYLDPDDKTRLEEPAVGFPMLKVKDIKSAYSHWQESKPILDKMVKQTRASSGVIWNSFKELEESELETVIREIPAPSFLIPLPKHYRASSTSLLDTDRSVFQWLDQQPPSSVLYVGFGSQSEVDPKDFLEIARGLVDSKQSFLWLVRPGFVKGSTWIEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 94 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPIINKHGADEFRKELESLMLASGSDEEVACLITDALWYFAQSVADSLNLRRLVLQTGSLFNFHCLVSLPKFDELGYLDPDDKTRLEEPAVGFPMLKVKDIKSAYSHWQESKPILEKMVKQTRASSGVIWNSFKELEESELETVQREIPAPSFLIPLPKHYTASSTSLLDTDRSVFQWLDQQPPSSVLYVGFGSQSEVDPKDFLEIARGLVDSKQSFLWLVRPGFVKGSTWVEPLPDGFLGEKGRIVKSAPQQEVLAHGAIGAFWTHGGWNGTMESVCEGVPMIFSDFGLDQPLNARYMSDVLHVGVYLENGWERGEIANAIRRVMVDEEGEYMRQNARVLKQKADRSIMKGGSSYESLESLVSYISSL

SEQ ID NO: 95 - protein, 457 aa, synthetic construct
MPNTNTTVVRRRRIILFPVPFPGHLNPMLQLANVLYRKGFSITIFHTNFNKPKTSNYPHFTFRFILDNDPQDEWLRNLPTHGPGVGARIPVINEHGADEFRKELESLMLASPSDEEVACVITDALWYFAQSVADSLNLKRLVLQTGSLFNFHCLVSLPKFDELGYLDPEDKHRPEEPAVGFPMLKVKDIRRAYSHWQESKPILEKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGSTWIELLPDGFLGEKGRIVKSAPQQEVLAHRAIGAFWTHGGWNGTMESVCEGVPMIFSDFGLDQPLNARYMSEVLHVGVYLENGWERGEIANAIRRVMVDPEGEVMRQNARVLKQKADRSIAPGGSSYESLERLVSYISSL

SEQ ID NO: 96 - protein, 457 aa, synthetic construct
MPNTNTTVVRRRRIILFPVPFPGHLNPMLQLANVLYRKGFAITIFHTNFNKPKTSNYPHFQFRFILDNDPQDEWLRNLPTHGPGVGARIPIINEHGADEFRKELELLMLASPSDEEVACVITDALWYFAQSVADSLNLKRLVLQTGSLFNFHCLVSLPKFRELGYLDPEDKHRLEEPAVGFPMLKVKDIRRAYSHWQESKPILDKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGSTWIELLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 97 - protein, 457 aa, synthetic construct
MPNTNTTVVRRRRIILFPVPFPGHLNPMLQLANVLYSKGFSITIFHTNFNKPKTSNYPHFQFRFILDNDPQDEWLRNLPTHGPGVGARIPQINKHGADEFRKELESLMLASPSDEEVACVITDALWYFAQSVADSLNLKRLVLQTGSLFNFHCLVSLPKFDELGYLDPEDKHRPEEPAVGFPMLKVKDIKRAYSHWQESKPILGKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGSTWIELLPDGFLGEKGRVVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 98 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKCAYSHWQESKPILGNMVKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 99 - protein, 457 aa, synthetic construct
MPNTNTTVVRRRRIILFPVPFPGHLNPMLQLANVLYSKGFSITIFHTNFNAPKTSLYPHFTFRFILDNDPQTEWLRNLPTHGPGVGARIPVINEHGADEFRKELELLMLASPSDEEVSCVITDALWYFAQSVADSLNLKRLVLQTGSLFNFHCLVSLPKFDELGYLDPEDKHRLEEPVVGFPMLKVKDIKRAYSHWQESKPILDKMVKQTRASSGVIWNSFKELEESELETVWREIPAPSFLLPLPKHYRASSTSLLDTDRSTAQWLDQQPPSSVLYVSFGSQSEVDPKDFLEIARGLVASKQSFLWLVRPGFVKGYTWIELLPDGFLGEKGRVVKSAPQQEVLAHGAIGAFWTHGGWNGTMEAVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 100 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSELDAKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 101 - protein, 457 aa, synthetic construct
MPNTNTTVVRRRRIILFPVPFPGHLNPMLQLANVLYSKGFSITIFHTNFNAPKTSNYPHFTFRFILDNDPQDEWLRNLPTHGPGVGARIPQINKHGADEFRKELESAMLASPSDEEVACVITDALWYFAQSVADSLNLKRLVLQTGSLFNFHCLVSLPKFRELGYLDPEDKHRLEEPAVGFPMLKVKDIKRAYSHWQESKPILGKMIEQTRASSGVIWNSFKELEESELETVWREIPAPSFLLPLPKHYRASSTSLLDTDRSVFQWLDQQPPSSVLYVSFGSQSEVDPKDFLEIARGLVASKQSFLWLVRPGFVKGSTWIELLPDGFLGEKGRVVKSAPQQEVLAHGAIGAFWTHGGWNGTMESVCEGVPMIFSDFGLDQPLNARYMSEVLHVGVYLENGFERGEIANAIRRVMVDEEGEVMRQNARKLKQKADRSIAPGGSSYESLERLVSYISSL

SEQ ID NO: 102 - protein, 457 aa, synthetic construct
MPNTNTTVVRRRRVILFPVPFPGHLNPMLQLANVLYSKGFSITILHTNFNKPKTSNYPHFTFRFILDNDPQDEWLRNLPTHGPGVGARIPVINKHGADEFRKELESLMLASPSDEEVACVITDALWYFAQSVADSLNLKRLVLQTGSLFNFHCLVSLPKFDELGYLDPEDKHRLEEPAVGFPMLKVKDIKRAYSHWQESKPILDKMVKETRASSGVIWNSFKELEESELETVWREIPAPSFLLPLPKHYRASSTSLLDTDRTVFQWLDQQPPSSVLYVSFGSQSEVDPKDFLEIARGLVASKQSFLWLVRPGFVKGSTWIELLPDGFLGEKGRVVKSAPQQEVLAHKAIGAFWTHGGWNGTMESVCEGVPMIFSDFGLDQPLNARYMSEVLHVGVYLENGWERGEIANAIRRVMVDEEGEVMRQNARKLKQKLDRSIAPGGSSYESLERLVSYISSL

SEQ ID NO: 103 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMIKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERQEIANAIRRVMVDEEGEYIRQNARVLKQKADVSLMKGGSSYESLESLVSYISSL

SEQ ID NO: 104 - protein, 457 aa, synthetic construct
MPNTNTTVVRRRRIILFPVPFPGHLNPMLQLANVLYRKGFSITIFHTNFNKPKTSLYPHFTFRFILDNDPQDEWLRNLPTHGPGVGARIPVINKHGADEFRKELELLMLASPSDEEVACVITDALWYFAQSVADSLNLKRLVLQTGSLFNFHCLVSLPKFDELGYLDPEDKHRLEEPVVGFPMLKVKDIRRAYSHWQESKPILDKMVKQTRASSGVIWNSFKELEESELETVQREIPAPSFLLPLPKHYRASSTSLLDTDRSTFQWLDQQPPSSVLYVGFGSQSEVDPKDFLEIARGLVASKQSFLWLVRPGFVKGYTWIELLPDGFLGEKGRVVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKDKADRSIAPGGSSYESLERLVSYISSL

SEQ ID NO: 105 - protein, 457 aa, synthetic construct
MPNTNTTVRRRRRIILFPVPFPGHINPILQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKCAYSHWQESKPILGKMVKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 106 - protein, 457 aa, synthetic construct
MPNTNPTVVRRRRIILFPVPFPGHLNPMLQLANVLYSKGFSITIFHTNFNKPKTSNYPHFTFRFILDNDPQDEWLRNLPTHGPGVGARIPVINEHGADEFRKELESLMLASPSDEEVSCVITDALWYFAQPVADSLNLPRLVLQTGSLFNFHCLVSLPKFLELGYLDPEDKHRLEEPVVGFPMLKVKDIKRAYSHWQESKPILDKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGSTWIELLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKRKADRSIAPGGSSYESLERLVSYISSL

SEQ ID NO: 107 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRPLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSEVDAKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 108 - protein, 457 aa, synthetic construct
MPNTNTTTVRRRRIILFPVPFPGHLNPMLQLANVLYRKGFSITIFHTNFNKPKTSNYPHFTFRFILDNDPQDEWLRNLPTHGPGVGARIPQINEHGADEFRKELELLMLDSPSDEEVACVITDALWYFAQSVADSLNLKRLVLQTGSLFNFHCLVSLPKFDELGYLDPEDKHRLEEPAVGFPMLKVKDIRRAYSHWQESKPILGKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGYTWIELLPDGFLGEKGRVVKSAPQQEVLAHGAIGAFWTHGGWNGTMEAVCEGVPMIFSDFGLDQPLNARYMSEVLHVGVYLENGFERGEIANAIRRVMVDEEGEVMRQNARVLKQKADRSIAPGGSSYESLERLVSYISSL

SEQ ID NO: 109 - protein, 457 aa, synthetic construct
MPNTNPTVVRRRRIILFPVPFPGHLNPMLQLANVLYSKGFSITIFHTNFNKPKTSNYPHFQFRFILDNDPQTEWLRNLPTHGPGVGARIPQINEHGADEFRKELELMMLASPSDEEVSCVITDALWYFAQSVADSLNLKRLVLQTGSLFNFHCLVSLPKFDELGYLDPEDKHRLEEPVVGFPMLKVKDIRRAYSHWQESKPILDKMVKETRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGSTWIELLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 110 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTKASSGVIWNSFKELEESELETVIREIPAPSFLIPLPKHLTASSTSLLDTDRTVFQWLDQQPPSSVLYVSFGSQSEVDAKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 111 - protein, 457 aa, synthetic construct
MPNTNTTVVRRRRIILFPVPFPGHLNPMLQLANVLYSKGFSITIFHTNFNKPKTSNYPHFTFRFILDNDPQDEWLRNLPTHGPGVGARIPQINEHGADEFRKELELLMLDSPSDEEVSCVITDALWYFAQPVADSLNLKRLVLQTGSLFNFHCLVSLPKFRELGYLDPEDKHRLEEPAVGFPMLKVKDIKRAYSHWQESKPILDKMVEQTRASSGVIWNSFKELEESELETVWREIPAPSFLLPLPKHYRASSTSLLDTDRSTFQWLDQQPPSSVLYVGFGSQSEVDPKDFLEIARGLVASKQSFLWLVRPGFVKGSTWIELLPDGFLGEKGRIVKSAPQQEVLAHKAIGAFWTHGGWNGTMESVCEGVPMIFSDFGLDQPLNARYMSEVLHVGVYLENGWERGEIANAIRRVMVDEEGEHMRQNARVLKQKADRSIAPGGSSYESLERLVSYISSL

SEQ ID NO: 112 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESRESLVSYISSL

SEQ ID NO: 113 - protein, 457 aa, synthetic construct
MPNTNTTTVRRRRVILFPVPFPGHLNPMLQLANVLYSKGFSITIFHTNFNKPKTSLYPHFTFRFILDNDPQDEWLRNLPTHGPGVGARIPQINKHGADEFRKELESLMLASPSDEEVSCVITDALWYFAQPVADSLNLPRLVLQTGSLFNFHCLVSLPKFDELGYLDPEDKHRLEEPAVGFPMLKVKDIKRAYSHWQESKPILGKMVKQTRASSGVIWNSFKELEESELETVWREIPAPSFLLPLPKHYRASSTSLLDTDRSVFQWLDQQPPSSVLYVSFGSQSEVDPKDFLEIARGLVASKQSFLWLVRPGFVKGYTWIELLPDGFLGEKGRVVKSAPQQEVLAHGAIGAFWTHGGWNGTMESVCEGVPMIFSDFGLDQPLNARYMSEVLHVGVYLENGWERGEIANAIRRVMVDPEGEVMRQNARVLKQKADRSIAPGGSSYESLERLVSYISSL

SEQ ID NO: 114 - protein, 457 aa, synthetic construct
MPNTNTTVVRRRRIILFPVPFPGHLNPMLQLANVLYSKGFSITIFHTNFNAPKTSNYPHFTFRFILDNDPQTEWLRNLPTHGPGVGARIPIINEHGADEFRKELESLMLASPSDEEVACLITDALWYFAQSVADSLNLKRLVLQTGSLFNFHCLVSLPKFDELGYLDPEDKHRLEEPAVGFPMLKVKDIKRAYSHWQESKPILGKMIKQTRASSGVIWNSFKELEESELETVQREIPAPSFLLPLPKHYRASSTSLLDTDRSVFQWLDQQPPSSVLYVGFGSQSEVDPKDFLEIARGLVASKQSFLWLVRPGFVKGYEWIELLPDGFLGEKGRVVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKDKADRSIAPGGSSYESLERLVSYISSL

SEQ ID NO: 115 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPRSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 116 - protein, 457 aa, synthetic construct
MPNTNTTVVRRRRVILFPVPFPGHLNPMLQLANVLYRKGFSITILHTNFNKPKTSNYPHFTFRFILDNDPQDEWLRNLPTHGPGVGARIPVINKHGADEFRKELESLMLASPSDEEVACVITDALWYFAQSVADSLNLKRLVLQTGSLFNFHCLVSLPKFDELGYLDPEDKHRLEEPAVGFPMLKVKDIKRAYSHWQESKPILDKMVKQTRASSGVIWNSFKELEESELETVWREIPAPSFLLPLPKHYRASSTSLLDTDRTVFQWLDQQPPSSVLYVSFGSQSEVDPKDFLEIARGLVASKQSFLWLVRPGFVKGSTWIELLPDGFLGEKGRVVKSAPQQEVLAHKAIGAFWTHGGWNGTMESVCEGVPMIFSDFGLDQPLNARYMSEVLHVGVYLENGWERGEIANAIRRVMVDEEGEVMRQNARKLKQKLDRSIAPGGSSYESLERLVSYISSL

SEQ ID NO: 117 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKCAYSHWQESKPILGNMVKQTRASSGVIWNSFKELEESELETVIREIPAPSFLIPLPKHLTASSTSLLDTDRTVFQWLDQQPPSSVLYVSFGSQSEVDAKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 118 - protein, 457 aa, synthetic construct
MPNTNTTVVRRRRIILFPVPFPGHLNPMLQLANVLYSKGFSITIFHTNFNKPKTSNYPHFTFRFILDNDPQDEWLRNLPTHGPGVGARIPVINEHGADEFRKELELLMLASPSDEEVACVITDALWYFAQSVADSLNLKRLVLQTGSLFNFHCLVSLPKFLELGYLDPEDKHRLEEPAVGFPMLKVKDIKRAYSHWQESKPILGKMIEETRASSGVIWNSFKELEESELETVWREIPAPSFLLPLPKHYRASSTSLLDTDRSVAQWLDQQPPSSVLYVGFGSQSEVDPKDFLEIARGLVASKQSFLWLVRPGFVKGYTWIELLPDGFLGEKGRIVKSAPQQEVLAHGAIGAFWTHGGWNGTMEAVCEGVPMIFSDFGLDQPLNARYMSEVLHVGVYLENGWERGEIANAIRRVMVDEEGEVMRQNARVLKDKADRSIAPGGSSYESLERLVSYISSL

SEQ ID NO: 119 - protein, 457 aa, synthetic construct
MPNTNTTVVRRRRVILFPVPFPGHLNPMLQLANVLYSKGFSITIFHTNFNAPKTSNYPHFTFRFILDNDPQDEWLRNLPTHGPGVGARIPQINKHGADEFRKELESLMLASPSDEEVACVITDALWYFAQSVADSLNLKRLVLQTGSLFNFHCLVSLPKFRELGYLDPEDKHRLEEPAVGFPMLKVKDIRRAYSHWQESKPILGKMITQTRASSGVIWNSFKELEESELETVWREIPAPSFLLPLPKHYRASSTSLLDTDRSVFQWLDQQPPSSVLYVSFGSQSEVDPKDFLEIARGLVASKQSFLWLVRPGFVKGSTWIELLPDGFLGEKGRVVKSAPQQEVLAHGAIGAFWTHGGWNGTMEAVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 120 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERREIANAIRRVMVDEEGEYIRQNARVLKQKADVSLMKGGSSYESLESLVSYISSL

SEQ ID NO: 121 - protein, 457 aa, synthetic construct
MPNTNPTVVRRRRIILFPVPFPGHLNPMLQLANVLYSKGFAITIFHTNFNKPKTSLYPHFQFRFILDNDPQDEWLRNLPTHGPGVGARIPVINEHGADEFRKELESLMLASPSDEEVSCVITDALWYFAQPVADSLNLKRLVLQTGSLFNFHCLVSLPKFDELGYLDPEDKHRLEEPVVGFPMLKVKDIKRAYSHWQESKPILDKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGSTWIELLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 122 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESSESLVSYISSL

SEQ ID NO: 123 - protein, 457 aa, synthetic construct
MPNTNTTTVRRRRIILFPVPFPGHLNPMLQLANVLYSKGFSITILHTNFNAPKTSLYPHFTFRFILDNDPQDEWLRNLPTHGPGVGARIPQINKHGADEFRKELELLMLASPSDEEVACVITDALWYFAQSVADSLNLKRLVLQTGSLFNFHCLVSLPKFDELGYLDPEDKHRLEEPAVGFPMLKVKDIRRAYSHWQESKPILGKMVKETRASSGVIWNSFKELEESELETVWREIPAPSFLLPLPKHYRASSTSLLDTDRSTFQWLDQQPPSSVLYVGFGSQSEVDPKDFLEIARGLVASKQSFLWLVRPGFVKGSTWIELLPDGFLGEKGRVVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 124 - protein, 457 aa, synthetic construct
MPNTNTTVVRRRRIILFPVPFPGHLNPMLQLANVLYRKGFSITIFHTNFNKPKTSNYPHFTFRFILDNDPQTEWLRNLPTHGPGVGARIPVINEHGADEFRKELELLMLASPSDEEVACVITDALWYFAQSVADSLNLKRLVLQTGSLFNFHCLVCLPKFDELGYLDPEDKHRLEEPVVGFPMLKVKDIKRAYSHWQESKPILGKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGYTWIELLPDGFLGEKGRVVKSAPQQEVLAHGAIGAFWTHGGWNGTMEAVCEGVPMIFSDFGLDQPLNARYMSEVLHVGVYLENGWERGEIANAIRRVMVDEEGEVMRQNARKLKQKADRSIAPGGSSYESLERLVSYISSL

SEQ ID NO: 125 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMTKQTRASSGVIWNSFKELEESELETVIREIPAPSFLIPLPKHLTASSTSLLDTDRTVFQWLDQQPPSSVLYVSFGSQSEVDAKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 126 - protein, 457 aa, synthetic construct
MPNTNTTVVRRRRIILFPVPFPGHLNPMLQLANVLYSKGFSITIFHTNFNAPKTSNYPHFTFRFILDNDPQDEWLRNLPTHGPGVGARIPQINKHGADEFRKELESAMLASPSDEEVACVITDALWYFAQSVADSLNLKRLVLQTGSLFNFHCLVSLPKFRELGYLDPEDKHRLEEPAVGFPMLKVKDIKRAYSHWQESKPILGKMIEQTRASSGVIWNSFKELEESELETVWREIPAPSFLLPLPKHYRASSTSLLDTDRSTFQWLDQQPPSSVLYVSFGSQSEVDPKDFLEIARGLVASKQSFLWLVRPGFVKGSTWIELLPDGFLGEKGRVVKSAPQQEVLAHGAIGAFWTHGGWNGTMESVCEGVPMIFSDFGLDQPLNARYMSEVLHVGVYLENGFERGEIANAIRRVMVDEEGEVMRQNARVLKQKADRSIAPGGSSYESLERLVSYISSL

SEQ ID NO: 127 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKCAYSHWQESKPILGKMVKQTRASSGVIWNSFKELEESELETVIREIPAPSFLIPLPKHLTASSTSLLDTDRTVFQWLDQQPPSSVLYVSFGSQSELDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 128 - protein, 457 aa, synthetic construct; identical to SEQ ID NO: 95

SEQ ID NO: 129 - protein, 457 aa, synthetic construct
MPNTNTTVVRRRRIILFPVPFPGHLNPMLQLANVLYSKGFSITIFHTNFNKPKTSNYPHFTFRFILDNDPQDEWLRNLPTHGPGVGARIPQINKHGADEFRKELELMMLASPSDEEVSCVITDALWYFAQSVADSLNLKRLVLQTGSLFNFHCLVSLPKFDELGYLDPEDKHRLEEPVVGFPMLKVKDIKRAYSHWQESKPILDKMVKETRASSGVIWNSFKELEESELETVQREIPAPSFLLPLPKHYTASSTSLLDTDRSVFQWLDQQPPSSVLYVGFGSQSEVDPKDFLEIARGLVASKQSFLWLVRPGFVKGSEWIELLPDGFLGEKGRIVKSAPQQEVLAHKAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 130 - protein, 457 aa, synthetic construct
MPNTNTTVRRRRRIILFPVPFPGHINPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSEVDEKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESVESLVSYISSL

SEQ ID NO: 131 - protein, 457 aa, synthetic construct
MPNTNTTVVRRRRIILFPVPFPGHLNPMLQLANVLYSKGFSITIFHTNFNAPKTSNYPHFTFRFILDNDPQDEWLRNLPTHGPGVGARIPVINEHGADEFRKELESLMLASPSDEEVSCVITDALWYFAQPVADSLNLKRLVLQTGSLFNFHCLVSLPKFRELGYLDPEDKHRLEEPAVGFPMLKVKDIKRAYSHWQESKPILDKMIEQTRASSGVIWNSFKELEESELETVWREIPAPSFLLPLPKHYRASSTSLLDTDPSVFQWLDQQPPSSVLYVGFGSQSEVDPKDFLEIARGLVASKQSFLWLVRPGFVKGSTWIELLPDGFLGEKGRIVKSAPQQEVLAHGAIGAFWTHGGWNGTMESVCEGVPMIFSDFGLDQPLNARYMSEVLHVGVYLENGWERGEIANAIRRVMVDEEGEVMRQNARVLKDKADRSIAPGGSSYESLERLVSYISSL

SEQ ID NO: 132 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPIINEHGADELRRELELLMLASEEDEEVSCLITDALWYFAQSVADSLNLRRLVLQTGSLFNFHCLVSLPQFDELGYLDPDDKHRLEEQASGFPMLKVKDIKSAYSHWQESKPILGKMVKQTRASSGVIWNSFKELEESELETVIREIPAPSFLIPLPKHLTASSTSLLDTDRTVFQWLDQQPPSSVLYVSFGSQSEVDAKDFLEIARGLVDSKQSFLWLVRPGFVKGSTWVEPLPDGFLGERGRIVKSAPQQEVLAHGAIGAFWTHGGWNGTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 133 - protein, 457 aa, synthetic construct
MPNTNTTTVRRRRIILFPVPFPGHLNPMLQLANVLYSKGFSITIFHTNFNKPKTSNYPHFTFRFILDNDPQTEWLRNLPTHGPGVGARIPQINEHGADEFRKELELLMLASPSDEEVACVITDALWYFAQSVADSLNLKRLVLQTGSLFNFHCLVSLPKFDELGYLDPEDKHRLEEPAVGFPMLKVKDIRRAYSHWQESKPILGKMIKQTRASSGVIWNSFKELEESELETVWREIPAPSFLLPLPKHYRASSTSLLDTDRSVFQWLDQQPPSSVLYVGFGSQSEVDPKDFLEIARGLVASKQSFLWLVRPGFVKGYTWIELLPDGFLGEKGRVVKSAPQQEVLAHGAIGAFWTHGGWNGTMEAVCEGVPMIFSDFGLDQPLNARYMSEVLHVGVYLENGFERGEIANAIRRVMVDEEGEVMRQNARVLKQKLDRSIAPGGSSYESLERLVSYISSL

SEQ ID NO: 134 - protein, 457 aa, synthetic construct
MPNTNTTVVRRRRVILFPVPFPGHLNPMLQLANVLYSKGFSITILHTNFNKPKTSNYPHFTFRFILDNDPQDEWLRNLPTHGPGVGARIPVINEHGADEFRKELESLMLASPSDEEVACVITDALWYFAQLVADSLNLKRLVLQTGSLFNFHCLVSLPKFDELGYLDPEDKHRLEEPAVGFPMLKVKDIKRAYSHWQESKPILGKMIEQTRASSGVIWNSFKELEESELETVWREIPAPSFLLPLPKHYRASSTSLLDTDRTTFQWLDQQPPSSVLYVGFGSQSEVDPKDFLEIARGLVASKQSFLWLVRPGFVKGSTWIELLPDGFLGEKGRIVKSAPQQEVLAHKAIGAFWTHGGWNGTMESVCEGVPMIFSDFGLDQPLNARYMSEVLHVGVYLENGWERGEIANAIRRVMVDEEGEVMRQNARVLKQKADRSIAPGGSSYESLERLVSYISSL

SEQ ID NO: 135 - protein, 457 aa, synthetic construct
MPNTNPTTVRRRTVVMFPVPFPGHLNPMLQLANVLYRRGFKIIILYTNFNAPKTSLYPHFQFRPILDNDPQPEWLRNLPTTGPGVGARIPVINKHGAEELRAALLECIAETPSDEELACVITDALWYFAQPVADELNLPLLVLQTGSLFNFHCLVCLPRFEELGLLDPDSPHADDAPVPGFPHLRVRDIRRAYSHIQESKPILMKMVEATKASSGVIWNSAKELEESELETIQREIPAPSFLLPLPKHYRASSTSLLDTDPSTAAWLDTQAPRSVLYVGFGSQSSFELEDALALADGLVASGQPFLWLVRPGFVKGKEWIEFLPDGYLGEKGRIVKSAPQQEVLAHPAIGAFWTHGGWNGTMEAVCEGVPMIFSDFGLDQPLNARYMTRVKKIGVYLDDGFVREDIVAAIHTVMRAPEGEEMRANAEALKAKLDRSIAPGGSSYESLERLQSYISSL

SEQ ID NO: 136 - protein, 457 aa, synthetic construct
MPNTNPTTVRRRTVVMFPVPFPGHLNPMLQLANVLYRRGFRIIILYTNFNAPKTSLYPHFQFRPILDNDPQPEWLRNLPTTGPGVGARIPVINKHGAEALRAALKECIAATPSDEEVACVITDALWYFAAPVAKELNLPLLVLQTGSLFNFHCLVCLPEFEALGLLDPDSPHADDAPVPGYPHLRVRDIRRAYSHIQESKPILMKMVEATKESSGVIWNSAKELEESELETIQREIPAPSFLLPLPKHYRASSTSLLDTDPSTAAWLDTQPPRSVLYVGFGSQSSFEEEEFLALARGLVASGQPFLWLVRPGFVKGKDWIELLPDGYLGEKGRIVKSAPQQEVLAHPAIGAFWTHGGWNGTMEAVCEGVPMIFSDFGLDQPLNARYMTEVKKIGVYLRDGFVEADIVAAIHTVMRAPEGEEMRANALALKEKLDRSIAPGGSSYESLERLQSYISSL

SEQ ID NO: 137 - protein, 457 aa, synthetic construct
MPNTNPTTVRRKTVVLFPVPFPGHLNPMLQLANVLYRRGFKIIILYMNFNAPKTSLYPHFEFRQILDNDPQPEWLRNLPTTGPGVGARIPVINKHGAPELEKALKECIAATPSDEELACLIADALWYFAQPVAKSLNIPLLVLQTGSLFNFHCLVCLPQFEELGLLDPDSTHAPDEPVPGFPHLRVADIRRAYSHIQESKPILMKMVEETKKSSGVIWNSAKELEESELETIQREIPAPSFLLPLPKHYRASSTSLLDTDPSTAAWLATQPPRSVLYVGFGSQSSFEEEDALAIADGLVASGQPVLWLVRPGFVKGHEWIEFLPDGYLGERGRIVKSAPQQEVLAHPAIGAFWTHGGWNGTMEAVCEGVPMIFSDFGLDQPLNARYMTEVLKIGVYLDNGFVREDIVAAIQRVMKDPFGEEMRANAEELKAKLDRSIAPGGSSYESLERLQSYISSL

SEQ ID NO: 138 - protein, 457 aa, synthetic construct
MPNTNPTTVRRKTVVIFPVPFPGHLNPMLQLANVLYRRGFKIVILYMNFNAPKTSLYPHFTFRPILDNDPQPEWLRNLPTTGPGVGARIPVINKHGAEELRKALEECIAETPSDEELACVIADALWYFAEPVAKSLNLPLLVLQTGSLFNFHCLVCLPRFIELGLLDPDSTHRPDEPVPGFPHLRVADIRRAYSHIQESKPILMKMVEATKASSGVIWNSAKELEESELETIQREIPAPSFLLPLPKHYRASSTSLLDTDPSTAAWLATQPPESVLYVGFGSQSSFELEEALALARGLVASGQPFLWLVRPGFVKGKEWIEFLPDGYLGERGRIVKSAPQQEVLAHPAIGAFWTHGGWNGTMEAVCEGVPMIFSDFGLDQPLNARYMTEVKKIGVYLRDGFVEEDIVAAIRRVMRDPEGKEYRANAKALKAKLDRSIAPGGSSYESLERLQSYISSL

SEQ ID NO: 139 - protein, 457 aa, synthetic construct
MPNTNPTTVRRKTVVLFPVPFPGHLNPMLQLANVLYRRGFKIIILYMNFNAPKTSLYPHFEFRPILDNDPQPEWLRNLPTTGPGVGARIPVINKHGAEELREALKKCIADTPSDEELACVIADALWYFAQPVAEELNLPLLVLQTGSLFNFHCLVCLPRFEELGILDPDSTLADDAPVPGFPHLRVRDIRRAYSHIQESKPILMKMVEETRKSSGVIWNSAKELEESELETIQREIPAPSFLLPLPKHYRASSTSLLDTDPSTAAWLATQPPRSVLYVGFGSQSSFEPEDFLALARGLVASGQPVLWLVRPGFVKGYDWIELLPDGYLGEKGRIVKSAPQQEVLAHPAIGAFWTHGGWNGTMEAVCEGVPMIFSDFGLDQPLNARYMTRVKKIGVYLEDGFVEERIVAAIHRVMRAPEGEVMAANARELAAKLDRSIAPGGSSYESLERLQSYISSL

SEQ ID NO: 140 - protein, 457 aa, synthetic construct
MPNTNPTTVRRRTVVMFPVPFPGHLNPMLQLANVLYRRGFRIIILYTNFNAPKTSLYPHFQFRQILDNDPQPEWLRNLPTTGPGVGARIPVINKHGAPQLREALLQCIAETPSDEELACVITDALWYFAQPVADELNLPLLVLQTGSLFNFHCLVCLPRFIELGILDPNSTLADDAPVPGFPILRVRDIRRAYSHIQESKPILMKMVEATKASSGVIWNSAKELEESELETIQREIPAPSFLLPLPKHYRASSTSLLDTDPSTAAWLATQPPRSVLYVGFGSQSSLELEDALRLARGLVASGVPFLWLVRPGFVKGLDWIEFLPDGYLGERGRIVKSAPQQEVLAHPAIGAFWTHGGWNGTMEAVCEGVPMIFSDFGLDQPLNARYMTEKLKIGVYLRDGFVEEDIVAAIHRVMRAPEGEEYRANAAALAAKLDRSIAPGGSSYESLERLQSYISSL

SEQ ID NO: 141 - protein, 457 aa, synthetic construct
MPNTNPTTVRRRTVVMFPVPFPGHLNPMLQLANVLYRRGFKIIILYTNFNAPKTSLYPHFQFRPILDNDPQPEWLRNLPTTGPGVGARIPVINKHGAPALEAALKECIAATPSDEELACVITDALWYFAQPVADKLNLPLLVLQTGSLFNFHCLVCLPRFEELGLLDPDSTHAPDAPVPGFPHLRVADIRRAYSHIQESKPILMKMVEATKASSGVIWNSAKELEESELETIQREIPAPSFLLPLPKHYRASSTSLLDTDPSTAAWLDTQPPRSVLYVGFGSQSSFEEADALALARGLVASGVPFLWLVRPGFVKGKAWIEFLPDGYLGERGRIVKSAPQQEVLAHPAIGAFWTHGGWNGTMEAVCEGVPMIFSDFGLDQPLNARYMTRVKKIGVYLENGFVEADIVAAIHTVMRAPEGEKYRENALALKEKLDRSIAPGGSSYESLERLQSYISSL

SEQ ID NO: 142 - protein, 457 aa, synthetic construct
MPNTNPTTVRRRTVVMFPVPFPGHLNPMLQLANVLYRRGFKIIILYTNFNAPKTSLYPHFQFRLILDNDPQPEWLRNLPTTGPGVGARIPVINKHGAEQLEAALKKCIAETPSDEELACVITDALWYFAQPVAEKLNLPLLVLQTGSLFNFHCLVCLPEFEALGLLDPDSTHAPDAPVPGFPHLRVRDIRRAYSHIQESKPILMKMVEATKKSSGVIWNSAKELEESELETIQREIPAPSFLLPLPKHYRASSTSLLDTDPSTAAWLATQPPRSVLYVGFGSQSSFEPADALALADGLVASGQPFLWLVRPGFVKGYAWTELLPDGYLGERGRIVKSAPQQEVLAHPAIGAFWTHGGWNGTMEAVCEGVPMIFSDFGLDQPLNARYMTRVKKIGVYLDDGFVTADIVAAIHTVMRAPEGEEYRANAEELAAKLDRSIAPGGSSYESLERLQSYISSL

SEQ ID NO: 143 - protein, 457 aa, synthetic construct
MPNTNPTTVRRRTVVMFPVPFPGHLNPMLQLANVLYRRGFRIIILYTNFNAPKTSLYPHFQFRPILDNDPQPEWLRNLPTTGPGVGARIPVINKHGAEQLRAALLECIAATPSDEELACVITDALWYFAEPVAKELNLPLLVLQTGSLFNFHCLVCLPEFEALGLLDPDSPHAPDEPVPGFPHLRVADIRRAYSHIQESKPILMKMVEATKNSSGVIWNSAKELEESELETIQREIPAPSFLLPLPKHYRASSTSLLDTDPSTAAWLDTQAPGSVLYVGFGSQSSLDLADAKALADGLVASGQPFLWLVRPGFVKGLDWIEFLPDGYLGEKGRIVKSAPQQEVLAHPAIGAFWTHGGWNGTMEAVCEGVPMIFSDFGLDQPLNARYMTEVKKIGVYLRDGFVTADIVAAIKTVMEAEEGKEYRANAAELKAKLDRSIAPGGSSYESLERLQSYISSL

SEQ ID NO: 144 - protein, 457 aa, synthetic construct
MPNTNPTTVRRRTVVMFPVPFPGHLNPMLQLANVLYRRGFKIIILYTNFNAPKTSLYPHFQFRPILDNDPQPEWLRNLPTTGPGVGARIPVINKHGAEELKKALEKCIAETPSDEELACLITDALWYFAEPVAKSLNIPLLVLQTGSLFNFHCLVCLPEFEALGLLDPDSTHADDAPVPGFPHLRVRDIRRAYSHIQESKPILMKMVEATKASSGVIWNSAKELEESELETIQREIPAPSFLLPLPKHYRASSTSLLDTDPSTAEWLATQPPESVLYVGFGSQSSFEEEEALAIADGLVASGVPVLWLVRPGFIKGKDWIEFLPDGYLGERGRIVKSAPQQEVLAHPAIGAFWTHGGWNGTMEAVCEGVPMIFSDFGLDQPLNAAYMTRVKKIGVYLRNGFVREDIVAAIHRVMRAPEGAEMRANARALAEKLDRSIAPGGSSYESLERLQSYISSL

SEQ ID NO: 145 - protein, 457 aa, synthetic construct
MPNTNPTTVRRRTVIMFPVPFPGHLNPMLQLANVLYRRGFRIVILYTNFNAPKTSLYPHFQFRQILDNDPQPEWLRNLPTTGPGVGARIPVINKHGAEELRAALEKCIADTPSDEEVACFITDALWYFALPVAKSLGLKRLVLQTGSLFNFHCLVCLPRFLELGYLDPDTPLADDEPVPGFPHLRVRDIRRAYSHIQESKPILMKMVEETRKSSGVIWNSAKELEESELETIQREIPAPSFLLPLPKHYRASSTSLLDTDPSTAAWLDTQPPGSVLYVGFGSQSSLEEEDFLRLARGLVASGQPFLWLVRPGFVKGKEWIELLPDGYGGERIRIVKSAPQQEVLAHPAIGAFWTHGGWNGTMEAVCEGVPMIFSDFGLDQPLNARYMTEVKKIGVYLEDGFIEEDIVAAINRVLREPEGEVMRANAQALKDKLDRSIAPGGSSYESLERLQSYISSL

SEQ ID NO: 146 - protein, 457 aa, synthetic construct
MPNTNPTTVRRRTVVMFPVPFPGHLNPMLQLANVLYRRGFRIVILYTNFNAPKTSLYPHFQFRLILDNDPQPEWLRNLPTTGPGVGARIPVINKHGAEELRKALLECIAATPSDEELACVITDALWYFAQPVAKSLNLPLLVLQTGSLFNFHCLVCLPEFEELGYLDPNSTHAPDEPVPGFPHLRVADIRRAYSHIQESKPILMKMVEETKKSSGVIWNSAKELEESELETIQREIPAPSFLLPLPKHYRASSTSLLDTDPSTAAWLDTQPPRSVLYVGFGSQSSFELADALALADGLVASGQPVLWLVRPGFVKGYEWIEFLPDGYLGEKGRIVKSAPQQEVLAHPAIGAFWTHGGWNGTMEAVCEGVPMIFSDFGLDQPLNARYMTEVLKIGVYLDNGFIRDDIVAAIRRVMVDPEGEVYRANAEELKAKLDRSIAPGGSSYESLERLQSYISSL

SEQ ID NO: 147 - protein, 457 aa, synthetic construct
MPNTNPTTVRRRTVVMFPVPFPGHLNPMLQLANVLYRRGFKIVILYTNFNAPKTSLYPHFQFRPILDNDPQPEWLRNLPTTGPGVGARIPVINKHGAPELRKALEECIAETPSDEELACVITDALWYFAQPVADSLNLPLLVLQTGSLFNFHCLVCLPEFEELGYLDPNSTHRDDEPVPGFPHLRVRDIRRAYSHIQESKPILMKMVEMTKKSSGVIWNSAKELEESELETIQREIPAPSFLLPLPKHYRASSTSLLDTDPSTAAWLATQPPRSVLYVGFGSQSSFEPEDFLALAEGLVASGQPFLWLVRPGFVKGYEWIELLPDGYLGEKGRIVKSAPQQEVLAHPAIGAFWTHGGWNGTMEAVCEGVPMIFSDFGLDQPLNARYMTKVKKIGVYLEDGFVKDKIVAAIRRVMVDPEGEEYRANARELKEKLDRSIAPGGSSYESLERLQSYISSL

SEQ ID NO: 148 - protein, 457 aa, synthetic construct
MPNTNPTTVRRRRVVMFPVPFPGHLNPMLQLANVLYRRGFEITILYTNFNAPKTSLYPHFQFVPILDNDPQPEWLRNLPTTGPGVGARIPVINKHGAEELRAALEACIAATPSDEELACVITDALWYFAQPVADSLNLKRLVLQTGSLFNFHCLVCLPKFLELGLLDPNSTHADDEPVPGFPHLRVKDIRRAYSHIQESKPILMKMVEETRKSSGVIWNSAKELEESELETIQREIPAPSFLLPLPKHYRASSTSLLDTDPSTAAWLDTQPPGSVLYVGFGSQSSLDLEDFLALADGLVASGQPFLWLVRPGFVKGKEWIEFLPDGYLGEKGRIVKSAPQQEVLAHPAIGAFWTHGGWNGTMEAVCEGVPMIFSDFGLDQPLNARYMTEVKKIGVYLDNGFVKEDIVAAIKTVMVAPEGAEMRANAQKLKDKLDRSIAPGGSSYESLERLQSYISSL

SEQ ID NO: 149 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEELEKALKECIAATPSDEEVACVITDALWYFATPVAKSLNLKRLVLQTGSLFNFHCLVCLPKFLELGLLDPNSKHAPDAPVPGFPHLKVKDIRRAYSHIQESKPILMKMVEETRKSSGVIWNSAKELEESELETIQREIPAPSFLLPLPKHYRASSTSLLDTDPSTAAWLDTQPPRSVLYVGFGSQSSLDPADFLALADGLVASGQPFLWLVRPGFVKGKEWIELLPDGYLGERGRIVKSAPQQEVLAHPAIGAFWTHGGWNGTMEAVCEGVPMIFSDFGLDQPLNARYMTEVKKIGVYLEDGFVPEAIVAAIHTVMRAPEGEEYRANARKLKDKLDRSIAPGGSSYESLERLQSYISSL

SEQ ID NO: 150 - protein, 457 aa, synthetic construct
MPNTNPTTVRRRRVVMFPVPFPGHLNPMLQLANVLYRRGFEITILYTNFNAPKTSLYPHFQFRQILDNDPQPEWLRNLPTTGPGVGARIPVINKHGAEELKAALEKCIAETPSDEEVACVITDALWYFAQPVAKSLNLKRLVLQTGSLFNFHCLVCLPEFLELGLLDPDSKHRDDEPVPGFPHLRVKDIRRAYSHIQESKPILMKMVEETRKSSGVIWNSAKELEESELETIQREIPAPSFLLPLPKHYRASSTSLLDTDPSTAAWLDTQPPRSVLYVGFGSQSSLDLADFLALAEGLVASGQPFLWLVRPGFVKGKEWIEALPDGFLGERGRIVKSAPQQEVLAHPAIGAFWTHGGWNGTMEAVCEGVPMIFSDFGLDQPLNARYMTEVKKIGVYLENGFVRDDIVAAIKEVMVAPKGEEYRANAQKLKDKLDRSIAPGGSSYESLERLQSYISSL

SEQ ID NO: 151 - protein, 457 aa, synthetic construct
MPNTNPTTVRRRTVIMFPVPFPGHLNPMLQLANVLYRRGFRIVILYTNFNAPKTSLYPHFQFRQILDNDPQPEWLRNLPTTGPGVGARIPVINKHGAPEFRKELERCIAETPSDEEVACVITDALWYFAQPVADSLNLPLLVLQTGSLFNFHCLVCLPEFEELGLLDPDSTHAPDEPVPGFPHLRVADIRRAYSHIQESKPILMKMVEATKASSGVIWNSAKELEESELETIQREIPAPSFLLPLPKHYRASSTSLLDTDPSTAEWLATQPPRSVLYVGFGSQSSFEPEDALALAEGLVASGQPFLWLVRPGFVKGYEWIELLPDGFLGEKGRIVKSAPQQEVLAHPAIGAFWTHGGWNGTMEAVCEGVPMIFSDFGLDQPLNARYMTKVKKIGVYLDNGFVPEDIVAAIHRVMRAPEGEEMRANAEALKAKLDRSIAPGGSSYESLERLQSYISSL

SEQ ID NO: 152 - protein, 457 aa, synthetic construct
MPNTNPTTVRRRTVIMFPVPFPGHLNPMLQLANVLYRRGFKIIILYTNFNAPKTSLYPHFQFRQILDNDPQPEWLRNLPTTGPGVGARIPVINKHGADELRKALLECIAETPSDEEVACVITDALWYFAQPVADSLNLPLLVLQTGSLFNFHCLVCLPRFEELGLLDPNSTHAPDAPVPGFPHLRVRDIRRAYSHIQESKPILMKMVEATKASSGVIWNSAKELEESELETIQREIPAPSFLLPLPKHYRASSTSLLDTDPSTAAWLATQPPGSVLYVGFGSQSSFELEDALAIARGLVASGQPVLWLVRPGFVKGYEWIEFLPDGFLGEKGRIVKSAPQQEVLAHPAIGAFWTHGGWNGTMEAVCEGVPMIFSDFGLDQPLNARYMTKVKKIGVYLENGFVEEDIVAAIKRVMVDPEGEVYRANAKALADKLDRSIAPGGSSYESLERLQSYISSL

SEQ ID NO: 153 - protein, 457 aa, synthetic construct
MPNTNPTTVRRRRVVMFPVPFPGHLNPMLQLANVLYRRGFEITILYTNFNAPKTSLYPHFQFVPILDNDPQPEWLRNLPTTGPGVGARIPVINKHGAAELEAALKACIAATPSDEEVACVITDALWYFAQPVAEKLGLKRLVLQTGSLFNFHCLVCLPRFLELGILDPNSTLADDAPVPGFPHLRVKDIRRAYSHIQESKPILMKMVEETRKSSGVIWNSAKELEESELETIQREIPAPSFLLPLPKHYRASSTSLLDTDPSTAAWLDTQPPESVLYVGFGSQSSLDEADFLALAEGLVASGQPFLWLVRPGFVKGKEWIELLPDGYLGERGRIVKSAPQQEVLAHPAIGAFWTHGGWNGTMEAVCEGVPMIFSDFGLDQPLNARYMTEVKKIGVYLENGFVKEDIVAAIHKVMRDPEGEEYRANAKKLKDKLDRSIAPGGSSYESLERLQSYISSL

SEQ ID NO: 154 - protein, 457 aa, synthetic construct
MPNTNPTTVRRRRVVMFPVPFPGHLNPMLQLANVLYRRGFEITILYTNFNAPKTSLYPHFQFRLILDNDPQPEWLRNLPTTGPGVGARIPVINKHGAPQLKEALLECIAETPSDEEVACVITDALWYFATPVAKSLNLKRLVLQTGSLFNFHCLVCLPEFLELGLLDPNSTHADDDPVPGFPHLRVKDIRRAYSHIQESKPILMKMVEETRKSSGVIWNSAKELEESELETIQREIPAPSFLLPLPKHYRASSTSLLDTDPSTAAWLDTQPPGSVLYVGFGSQSSLDEEDFLALADGLVASGKPFLWLVRPGFVKGKEWIELLPDGYLGEKGRIVKSAPQQEVLAHPAIGAFWTHGGWNGTMEAVCEGVPMIFSDFGLDQPLNARYMTEVKKIGVYLDNGFKKDDIVAAIKKVMEEEEGKEYRENAQKLKDKLDRSIAPGGSSYESLERLQSYISSL

SEQ ID NO: 155 - protein, 457 aa, synthetic construct
MPNTNPTTVRRRRVVMFPVPFPGHLNPMLQLANVLYRRGFEITILYTNFNAPKTSLYPHFQFRLILDNDPQPEWLRNLPTTGPGVGARIPVINKHGAEQLEAALKECIAETPSDEEVACVITDALWYFAQPVADKLNLKRLVLQTGSLFNFHCLVCLPEFLELGLLDPDSTHADDAPVPGFPHLRVKDIRRAYSHIQESKPILMKMVEETRRSSGVIWNSAKELEESELETIQREIPAPSFLLPLPKHYRASSTSLLDTDPSTAEWLDTQPPESVLYVGFGSQSSLDLEDFLKLAKGLVKSGQPFLWLVRPGFVKGKEWIEFLPDGYLGEKGRIVKSAPQQEVLAHPAIGAFWTHGGWNGTMEAVCEGVPMIFSDFGLDQPLNARYMTEVKKIGVYLDNGFVEEDIVAAIKKVMVEPEGEEMRANAKKLKDKLDRSIAPGGSSYESLERLQSYISSL

SEQ ID NO: 156 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEELRAALLECIAATPSDEELACVITDALWYFAQPVADELGLKRLVLQTGSLFNFHCLVCLPKFLELGYLDPNSTHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPPESVLYVGFGSQSSLDEADFLAIAEGLVASGQPFLWLVRPGFVKGKEWIELLPDGYLGEKGRIVKSAPQQEVLAHPAIGAFWTHGGWNGTMEAVCEGVPMIFSDFGLDQPLNARYMSDILKVGVYLDDGFVKDKIVEAIKTVMVDEEGKEYRANAKKLKDKLDRSIAPGGSSYESLERLQSYISSL

SEQ ID NO: 157 - protein, 457 aa, synthetic construct
MPNTNPTTVRRRRVIMFPVPFPGHLNPMLQLANVLYRRGFEITILHTNFNAPKTSLYPHFQFRLILDNDPQPEWLRNLPTTGPGVGARIPVINKHGAEQLEAALKQCIAETPSDEEVACLITDALWYFAQPVADSLNLKRLVLQTGSLFNFHCLVCLPKFLELGYLDPNSTLKDDEPVPGFPHLKVKDIRRAYSHIQESKPILMKMVEETRASSGVIWNSAKELEESELETIQREIPAPSFLLPLPKHYRASSTSLLDTDPSTAQWLDTQPPESVLYVGFGSQSSLDLEDFLAIAKGLVASGQPFLWLVRPGFVKGKEWIEFLPDGYLGEKGRIVKSAPQQEVLAHPAIGAFWTHGGWNGTMEAVCEGVPMIFSDFGLDQPLNARYMSDILKVGVYLDNGFVEADIVKAIKTVMVDPEGKEYRANAQKLKDKLDRSIAPGGSSYESLERLQSYISSL

SEQ ID NO: 158 - protein, 457 aa, synthetic construct
MPNTNPTTVRRRRVVMFPVPFPGHLNPMLQLANVLYRRGFEITILHTNFNAPKTSLYPHFQFVQILDNDPQPEWLRNLPTTGPGVGARIPVINKHGAEQLREALLKCIADTPSDEEVACLITDALWYFAQPVADSLNLKRLVLQTGSLFNFHCLVCLPKFLELGYLDPNSTLKDDEPVPGFPILKVKDIRRAYSHIQESKPILMKMVEETRASSGVIWNSAKELEESELETIQREIPAPSFLLPLPKHYRASSTSLLDTDPSTAQWLDTQPANSVLYVGFGSQSSLDPEDFLNIADGLVASGQPFLWLVRPGFVKGYEWIELLPDGYLGEKGRIVKSAPQQEVLAHPAIGAFWTHGGWNGTMEAVCEGVPMIFSDFGLDQPLNARYMSEKLKVGVYLDDGFVKDKIVAAINKVMKEEEGKEYRANAQKLKDKLDRSIAPGGSSYESLERLQSYISSL

SEQ ID NO: 159 - protein, 457 aa, synthetic construct
MPNTNPTTVRRRTVIMFPVPFPGHLNPMLQLANVLYRRGFRIVILYTNFNAPKTSLYPHFQFRQILDNDPQPEWLRNLPTTGPGVGARIPVINKHGAEEFRAALEQCIADTPSDEEVACVITDALWYFAQPVADSLGLKRLVLQTGSLFNFHCLVCLPRFLELGLLDPDTPLAPDEPVPGFPHLRVADIRRAYSHIQESKPILMKMVEETRKSSGVIWNSAKELEESELETIQREIPAPSFLLPLPKHYRASSTSLLDTDPSTAAWLDTQPPGSVLYVGFGSQSSLEPEEFLALADGLVASGQPFLWLVRPGFVKGHEWIELLPDGYLGEKGRIVKSAPQQEVLAHPAIGAFWTHGGWNGTMEAVCEGVPMIFSDFGLDQPLNARYMTEVLKIGVYLENGFIPEDIVAAIKTVMEAPEGEVMRANARALKDKLDRSIAPGGSSYESLERLQSYISSL

SEQ ID NO: 160 - protein, 457 aa, synthetic construct
MPNTNPTTVRRRTVVMFPVPFPGHLNPMLQLANVLYRRGFRIIILYTNFNAPKTSLYPHFQFRPILDNDPQPEWLRNLPTTGPGVGARIPVINKHGAEEFRKALEECIAATPSDEEVACVITDALWYFAQPVAKSLNLKRLVLQTGSLFNFHCLVCLPKFLELGLLDPDSKHADDEPVPGFPHLRVKDIRRAYSHIQESKPILMKMVEETKKSSGVIWNSAKELEESELETIQREIPAPSFLLPLPKHYRASSTSLLDTDPSTAAWLDTQPPRSVLYVGFGSQSSLEPEEFLALADGLVASGQPFLWLVRPGFVKGYEWIELLPDGYLGEKGRIVKSAPQQEVLAHPAIGAFWTHGGWNGTMEAVCEGVPMIFSDFGLDQPLNARYMTEVKKIGVYLDDGFVPEAIVAAIHRVMVAPEGEVMRANAQALKDKLDRSIAPGGSSYESLERLQSYISSL

SEQ ID NO: 161 - protein, 457 aa, synthetic construct
MPNTNPTTVRRRTVVMFPVPFPGHLNPMLQLANVLYRRGFRIIILYTNFNAPKTSLYPHFQFRQILDNDPQPEWLRNLPTTGPGVGARIPVINKHGAEELEKALKECIAETPSDEELACVITDALWYFAQPVADKLNLKRLVLQTGSLFNFHCLVCLPKFLELGLLDPNSTHADDEPVPGFPHLRVKDIRRAYSHIQESKPILMKMVEETKKSSGVIWNSAKELEESELETIQREIPAPSFLLPLPKHYRASSTSLLDTDPSTAAWLDTQPPESVLYVGFGSQSSLELADALALAEGLVRSGQPFLWLVRPGFVKGKAWIEFLPDGFLGEKGRIVKSAPQQEVLAHPAIGAFWTHGGWNGTMEAVCEGVPMIFSDFGLDQPLNARYMTEVKKIGVYLDDGFVVDKIVEAIKTVMVDEKGKEYRANAKALKDKLDRSIAPGGSSYESLERLQSYISSL

SEQ ID NO: 162 - protein, 457 aa, synthetic construct
MPNTNPTTVRRRRVVMFPVPFPGHLNPMLQLANVLYRRGFDIIILYTNFNAPKTSLYPHFQFRRILDNDPQPEWLRNLPTTGPGVGARIPVINKHGAEELRKALLECIAETPSDEELACLITDALWYFAQPVADELNLKRLVLQTGSLFNFHCLVCLPRFLELGYLDPNSTLADDAPVVGFPHLRVKDIRRAYSHIQESKPILMKMVEETKKSSGVIWNSAKELEESELETIQREIPAPSFLLPLPKHYRASSTSLLDTDPSTAAWLDTQPPRSVLYVGFGSQSSLELADALALADGLVASGQSFLWLVRPGFVKGLDWVEFLPDGYLGERGRIVKSAPQQEVLAHPAIGAFWTHGGWNGTMEAVCEGVPMIFSDFGLDQPLNARYMTDVLKIGVYLDNGFVRDEIVAAIHTVMRDPEGEVYRANAAALKDKLDRSIAPGGSSYESLERLQSYISSL

SEQ ID NO: 163 - protein, 457 aa, synthetic construct
MPNTNPTTVRRRRVVMFPVPFPGHLNPMLQLANVLYRRGFDIVILYTNFNAPKTSLYPHFQFRPILDNDPQPEWLRNLPTTGPGVGARIPVINKHGAPELRAALEQCIADTPSDEEVACVITDALWYFAQPVADELNLPLLVLQTGSLFNFHCLVCLPKFEELGLLDPNSKHRDDEPVPGFPHLRVRDIRRAYSHIQESKPILMKMVEATKASSGVIWNSAKELEESELETIQREIPAPSFLLPLPKHYRASSTSLLDTDPSTAAWLATQPPKSVLYVGFGSQSSFEPEEALEIAEGLVESKQPFLWLVRPGFVKGYEWIELLPDGFLGEKGRIVKSAPQQEVLAHPAIGAFWTHGGWNGTMEAVCEGVPMIFSDFGLDQPLNARYMTRVKKIGVYLDNGFIKEEIVKAIQTVMKDPEGAEYRANAQELKAKLDRSIAPGGSSYESLERLQSYISSL

SEQ ID NO: 164 - protein, 457 aa, synthetic construct
MPNTNPTTVRRRTVVMFPVPFPGHLNPMLQLANVLYRRGFKIIILYTNFNAPKTSLYPHFQFRPILDNDPQPEWLRNLPTTGPGVGARIPVINKHGAEELRKELEICIEETPSDEEVACVITDALWYFAQPVADELNLPLLVLQTGSLFNFHCLVCLPEFEKLGYLDPNSKHKDDEPVPGFPMLKVKDIRRAYSHIQESKPILMKMVEATRRSSGVIWNSAKELEESELETIQREIPAPSFLLPLPKHYRASSTSLLDTDPSTAAWLDTQPPRSVLYVGFGSQSSLDLDDALALARGLVASGQPVLWLVRPGFVKGLDWIEFLPDGFLGERGRIVKSAPQQEVLAHPAIGAFWTHGGWNGTMEAVCEGVPMIFSDFGLDQPLNARYMTEKLKVGVYLDDGFVEDAIVAAIHTVMRAPEGEEMRANAEELKAKLDRSIAPGGSSYESLERLQSYISSL

SEQ ID NO: 165 - protein, 457 aa, synthetic construct
MPNTNPTTVRRRRVVMFPVPFPGHLNPMLQLANVLYRRGFEITILHTNFNAPKTSLYPHFQFRLILDNDPQPEWLRNLPTTGPGVGARIPVINKHGAEELRAALLECIADTPSDEEVACVITDALWYFAQPVADSLGLKRLVLQTGSLFNFHCLVCLPKFLELGYLDPNTPLADDAPVPGFPHLRVKDIRRAYSHIQESKPILMKMVEETRKSSGVIWNSAKELEESELETIQREIPAPSFLLPLPKHYRASSTSLLDTDPSTAAWLDTQPPKSVLYVGFGSQSSLDLADFLAIADGLRASGQPFLWLVRPGFVKGLEWIEALPDGYGGERIRIVKSAPQQEVLAHPAIGAFWTHGGWNGTMEAVCEGVPMIFSDFGLDQPLNARYMTEILKVGVYLDNGFVPADIVAAVKRVMRDPEGEEMRANAQKLKDKLDRSIAPGGSSYESLERLQSYISSL

SEQ ID NO: 166 - protein, 457 aa, synthetic construct
MPNTNPTTVRRRTVIMFPVPFPGHLNPMLQLANVLYRRGFRIIILYTNFNAPKTSLYPHFQFRPILDNDPQPEWLRNLPTTGPGVGARIPVINKHGADEFEKALKECMAETPSDEEVACVITDALWYFATPVAKSLNLKRLVLQTGSLFNFHCLVCLPKFLELGILDPNTTLAPDAPVPGFPHLRVKDIRRAYSHIQESKPILMKMVEETKASSGVIWNSAKELEESELETIQREIPAPSFLLPLPKHYRASSTSLLDTDPSTAQWLDTQPPESVLYVGFGSQSSLEPEDALALARGLVASGQPFLWLVRPGFVKGHDWIELLPDGFLGERGRIVKSAPQQEVLAHPAIGAFWTHGGWNGTMEAVCEGVPMIFSDFGLDQPLNARYMTEVKKIGVYLDDGFREEDIIAAIRTVMRDPRGEEMRANARALKDKLDRSIAPGGSSYESLERLQSYISSL

SEQ ID NO: 167 - protein, 457 aa, synthetic construct
MPNTNPTTVRRRTVVMFPVPFPGHLNPMLQLANVLYRRGFKIIILYTNFNAPKTSLYPHFQFRPILDNDPQPEWLRNLPTTGPGVGARIPVINKHGAEELRKALEECIAATPSDEEVACVITDALWYFAQPVADELNLKRLVLQTGSLFNFHCLVCLPKFLELGLLDPNSKHKDDEPVPGYPMLRVKDIRRAYSHIQESKPILMKMVEETKKSSGVIWNSAKELEESELETIQREIPAPSFLLPLPKHYRASSTSLLDTDPSTAAWLDTQPPESVLYVGFGSQSSLEPEDALEIARGLVASKQPFLWLVRPGFVKGHAWTELLPDGFLGERGRIVKSAPQQEVLAHKAIGAFWTHGGWNGTMEAVCEGVPMIFSDFGLDQPLNARYMTEKLKIGVYLENGFVEEDIVAAVHEVMVAPKGKEYRKNAKELKDKLDRSIAPGGSSYESLERLQSYISSL

SEQ ID NO: 168 - protein, 457 aa, synthetic construct
MPNTNPTTVRRRRVVMFPVPFPGHLNPMLQLANVLYRRGFEITILYTNFNAPKTSLYPHFQFRFILDNDPQPEWLRNLPTTGPGVGARIPVINKHGAEELRKALEECIAETPSDEELACLITDALWYFAQPVAKSLNIKRLVLQTGSLFNFHCLVCLPKFLELGYLDPNTTLKDDEPVVGFPHLKVKDIRRAYSHIQESKPILMKMVEETRKSSGVIWNSAKELEESELETIQREIPAPSFLLPLPKHYRASSTSLLDTDPSTAAWLDTQPPESVLYVGFGSQSSLDEEDFLKLVKGLVASKQPFLWLVRPGFVKGKEWIELLPDGYLGEKGRIVKSAPQQEVLAHPAIGAFWTHGGWNGTMEAVCEGVPMIFSDFGLDQPLNARYMTDVKKIGVYLENGFVEDEIVAAIKKVMVDEEGKEYAANARKLKDKLDRSIAPGGSSYESLERLQSYISSL

SEQ ID NO: 169 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFRKALEELIAASESDEEVSCLITDALWYFAQSVADSLNLKRLVLQTGSLFNFHCLVSLPKFDELGYLDPDDKTRLEEPAVGFPMLKVKDIKSAYSHWQESKPILMKMVKQTRASSGVIWNSFKELEESELETVQREIPAPSFLIPLPKHYRASSTSLLDTDRSVFQWLDQQPPSSVLYVGFGSQSEVDPKDFLEIARGLVLSGQSFLWLVRPGFVKGSTWIEPLPEGFLGEKGRVVKSAPQQEVLAHGAIGAFWTHGGWNGTMESVCEGVPMIFSDFGLDQPLNARYMSEVLHVGVYLENGWDPEAIAAAIRLVMVDEAGEPMRQNARVLKQKADRSIMKGGSSYESLESLVSYISSL

SEQ ID NO: 170 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKLLEELIAASESDEEVSCLITDALWYFAQSVADSLNLRRLVLQTGSLFNFHCLVSLPKFDELGYLDPDDKTRLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLEIARGLVDSGQSFLWLVRPGFVKGSTWIEPLPEGFLGEKGRIVKSAPQQEVLAHGAIGAFWTHGGWNGTMESVCEGVPMIFSDFGLDQPLNARYMSEVLHVGVYLENGWNPKDIANAIRYVMTDEAGEPMRQNARVLKQKADRSIMKGGSSYESLESLVSYISSL

SEQ ID NO: 171 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKLLEELIAASESDEEVSCLITDALWYFAQSVADSLNLKRLVLQTGSLFNFHCLVSLPKFDELGYLDPDDKTDLDKPAVGFPMLKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDPKDFLAIAEGLVKSGQSFLWLVRPGFVKGSTWIEPLPEGFLGEKGRVVKSAPQQEVLAHGAIGAFWTHGGWNGTMEAVCEGVPMIFSDFGLDQPLNARYMSDVLHVGVYLENGWDPDAIAAAIRRVMVDPEGEPMRENARVLKQKADRSIMKGGSSYESLESLVSYISSL

SEQ ID NO: 172 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKMLEELMAASESDEEVSCLITDALWYFAQSVADSLNLRRLVLQTGSLFNFHCLVSLPKFDELGYLDPDDKTGLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLAIAEGLVKSGQSFLWLVRPGFVKGSTWIEPLPEGFLGEKGRIVKSAPQQEVLAHGAIGAFWTHGGWNGTMESVCEGVPMIFSDFGLDQPLNARYMSEVLHVGVYLENGWDPDAIAAAIRLVMVDEAGEPMRENARVLKQKADRSIMKGGSSYESLESLVSYISSL

SEQ ID NO: 173 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKELELLMEASESDEEVSCLITDALWYFAQSVADSLNLRRLVLQTGSLFNFHCLVSLPKFDELGYLDPDDKTRLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPADFLTIADGLVASGQSFLWLVRPGFVKGSTWIEPLPDGYLGEKGRIVKSAPQQEVLAHGAIGAFWTHGGWNGTMESVCEGVPMIFSDFGLDQPLNARYMSEVLHVGVYLENGWDPAAIAAAIRRVMVDESGEYMRQNARVLKQKADRSIMKGGSSYESLESLVSYISSL

SEQ ID NO: 174 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFEKLLKELMEASESDEEVSCLITDALWYFAQSVADSLNLKRLVLQTGSLFNFHCLVSLPKFDELGYLDPDDKTRLEEPAVGFPMLKVKDIKSAYSHWQESKPILMKMVKQTRASSGVIWNSFKELEESELETVQREIPAPSFLIPLPKHYRASSTSLLDTDRSVFQWLDQQPPSSVLYVGFGSQSEVDPKDFLAIAEGLVKSGQSFLWLVRPGFVKGSTWIEPLPEGFLGEKGRVVKSAPQQEVLAHGAIGAFWTHGGWNGTMESVCEGVPMIFSDFGLDQPLNARYMSEVLHVGVYLENGWNPDEIAEAIRRVMVDESGEPMRENARVLKQKADRSIMKGGSSYESLESLVSYISSL

SEQ ID NO: 175 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELEELIEASESDEEVSCLITDALWYFAQSVADSLNLKRLVLQTGSLFNFHCLVSLPKFDELGYLDPDDKTNLDEPAVGFPMLKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDPADFLTIAKGLVDSKQSFLWLVRPGFVKGSTWIEPLPEGFLGEKGRVVKSAPQQEVLAHGAIGAFWTHGGWNGTMEAVCEGVPMIFSDFGLDQPLNARYMSDVLHVGVYLENGWDPAAIANAIRYVMTDEAGEPMRENARVLKQKADRSIMKGGSSYESLESLVSYISSL

SEQ ID NO: 176 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFEKLLKELIAASESDEEVSCLITDALWYFAQSVADKLNLKRLVLQTGSLFNFHCLVSLPKFDELGYLDPDDKTRLEEPAVGFPMLKVKDIKSAYSHWQESKPILMKMVKQTRASSGVIWNSFKELEESELETVQREIPAPSFLIPLPKHYRASSTSLLDTDRSVFQWLDQQAPSSVLYVGFGSQSEVDPEDFLKIAQGLVDSGQSFLWLVRPGFVKGSTWIEPLPEGFLGEKGRVVKSAPQQEVLAHGAIGAFWTHGGWNGTMESVCEGVPMIFSDFGLDQPLNARYMSEVLHVGVYLENGWNPEDIANAIRLVMTDEAGQPMRDNARVLKQKADRSIMKGGSSYESLESLVSYISSL

SEQ ID NO: 177 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGAERFRKLLEELMAASESDEEVSCLITDALWYFAQSVADSLNLKRLVLQTGSLFNFHCLVSLPKFDELGYLDPDDKTNLEEPAEGFPMLKVKDIKSAYSHWQESKPILMKMVKQTRASSGVIWNSFKELEESELETVQREIPAPSFLIPLPKHYRASSTSLLDTDRSVFQWLDTQPPESVLYVGFGSQSEVDPKDFLTIAEGLVKSGQSFLWLVRPGFVKGSTWIEPLPPGFLGEKGRVVKSAPQQEVLAHGAIGAFWTHGGWNGTMESVCEGVPMIFSDFGLDQPLNARYMSEVLHVGVYLENGWDADAIAAAIRHVMVDESGEPIRENARVLKQKADRSIMKGGSSYESLESLVSYISSL

SEQ ID NO: 178 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFEKLLKELIAASESDEEVSCLITDALWYFAQSVADSLNLRRLVLQTGSLFNFHCLVSLPKFDELGYLDPDDKTNLDAPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPEDFLTIAEGLVKSGQSFLWLVRPGFVKGSTWIEPLPEGFLGEKGRIVKSAPQQEVLAHGAIGAFWTHGGWNGTMESVCEGVPMIFSDFGLDQPLNARYMSEVLHVGVYLENGWNPDDIAAAIRLVMTDPAGQPMRDNARVLKQKADRSIMKGGSSYESLESLVSYISSL

SEQ ID NO: 179 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFKKLLEELIAASESDEEVSCLITDALWYFAQSVADSLNLKRLVLQTGSLFNFHCLVSLPKFDELGYLDPDDKTRLDEPAEGFPMLKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDPADFLTIAEGLVASGQSFLWLVRPGFVKGSTWIEPLPPGFLGEKGRVVKSAPQQEVLAHGAIGAFWTHGGWNGTMEAVCEGVPMIFSDFGLDQPLNARYMSDVLKVGVYLENGWDPAAIAAAIRYVMVDEEGAPIRENARVLKQKADRSIMKGGSSYESLESLVSYISSL

SEQ ID NO: 180 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEEFKKALEELIAASESDEEVSCLITDALWYFAQSVADSLNLKRLVLQTGSLFNFHCLVSLPKFDELGYLDPDDKTNLEEPAEGFPMLKVKDIKSAYSHWQESKPILDKMVKQTRASSGVIWNSFKELEESELETVQREIPAPSFLIPLPKHYRASSTSLLDTDRSVFQWLDTQPPESVLYVGFGSQSEVDPEDFLKIAEGLVLSGQSFLWLVRPGFVKGSEWIEPLPEGFLGEKGRIVKSAPQQEVLAHGAIGAFWTHGGWNGTMESVCEGVPMIFSDFGLDQPLNARYMSDVLKVGVYLENGWDPEKIAEAIRYVMVDEKGEEIRNNARVLKQKADRSIMKGGSSYESLESLVSYISSL

SEQ ID NO: 181 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFEKLLKEYIEASESDEEVSCLITDALWYFAQSVADKLNLRRLVLQTGSLFNFHCLVSLPKFDELGYLDPDDKTNLEKPAEGFPMLKVKDIKRAYSHWQESKPILEKMVKQTKASSGVIWNSFKELEESELETVWREIPAPSFLIPLPKHYRASSTSLLDTDRSVFQWLDTQAPESVLYVGFGSQSEVDEADFLKIADGLVKSGISFLWLVRPGFVKGSTWIEPLPEGYLGDKGRIVKSAPQQEVLAHGAIGAFWTHGGWNGTMESVCEGVPMIFSDFGLDQPLNARYMSEVLKVGVYLENGWDPDEIAAAIRYVMRDEAGKPIRENAKVLKQKADRSIMKGGSSYESLESLVSYISSL

SEQ ID NO: 182 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFEKLLKELIAASESDETVSCLITDALWYFAQDVADSLNLKRLVLQTGSLFNFHCLVSLPKFDELGYLDPDDKTKLDEPAEGFPELKVKDIKSAYSHWQESKPILEKMVKQTRASSGVIWNSFKELEESELETVQREIPAPSFLIPLPKHYRASSTSLLDTDRSVFQWLDTQAPESVLYVGFGSQSEVDEEDFLTIVDGLVKSGISFLWLVRPGFVKGKTWIEPIPEGYLGEKGRVVKSAPQQEVLAHGAIGAFWTHGGWNGTMEAVCEGVPMIFSDFGLDQPLNARYMSDVLKVGVYLENGWDPEAIAKAIRYVMKDEAGKPIRENAKVLKQKADRSIMKGGSSYESLESLVSYISSL

SEQ ID NO: 183 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFRKLLEELMAASESDEEVSCLITDALWYFAQDVADSLNLRRLVLQTGSLFNFHCLVSLPKFDELGYLDPDDKTRLEEPAEGFPMLKVKDIKRAYSHWQESKPILEKMVKQTKASSGVIWNSFKELEESELETVWREIPAPSFLIPLPKHYRASSTSLLDTDRSVFQWLDTQPPRSVLYVGFGSQSEVDAADFLEIARGLVASGISFLWLVRPGFVKGSTWIEPLPDGFLGEKGRIVKSAPQQEVLAHGAIGAFWTHGGWNGTMESVCEGVPMIFSDFGLDQPLNARYMSEKLKVGVYLENGWDADEIAAAIRYVMVDPEGEPIRQNAEVLKQKADRSIMKGGSSYESLESLVSYISSL

SEQ ID NO: 184 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALEELMAASESDEEVSCLITDALWYFAQSVADSLNLKRLVLQTGSLFNFHCLVSLPKFDELGYLDPDNKERLEEPVEGFPELKVKDIKSAYSHWQESKPILDKMVKQTRASSGVIWNSFKELEESELETVQREIPAPSFLIPLPKHYRASSTSLLDTDRSVFQWLDQQPPESVLYVGFGSQSEVDPEDFLKIVEGLVKSGVSFLWLVRPGFVKGKEWIEPLPEGFLGEKGRIVKSAPQQEVLAHGAIGAFWTHGGWNGTMESVCEGVPMIFSDFGLDQPLNARYMSDVLKVGVYLENGWEPDEIAAAIRRVMRDPEGEVIRQNARVLKQKADRSIMKGGSSYESLESLVSYISSL

SEQ ID NO: 185 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFRKLLEELMAASESDEEVSCLITDALWYFAQDVADSLNLKRLVLQTGSLFNFHCLVSLPKFDELGYLDPDNKENLDEPAEGFPELKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDEKDFLAIAQGLVDSGQSFLWLVRPGFVKGKTWIEPLPDGFLGEKGRVVKSAPQQEVLAHGAIGAFWTHGGWNGTMEAVCEGVPMIFSDFGLDQPLNARYMSDVLKVGVYLENGWNPKEIAAAIRYVMVDEAGEPIRQNARVLKQKADRSIMKGGSSYESLESLVSYISSL

SEQ ID NO: 186 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEEFRKELEELMEASESDEEVSCLITDALWYFAQDVADSLNLKRLVLQTGSLFNFHCLVSLPKFDELGYLDPDNKTDLEKPAVGFPMLKVKDIKSAYSHWQESKPILDKMVKQTRASSGVIWNSFKELEESELETVQREIPAPSFLIPLPKHYRASSTSLLDTDRSVFQWLDTQPPESVLYVGFGSQSEVDPADFLAIADGLVASGQSFLWLVRPGFVKGHEWIEPLPDGYLGEKGRIVKSAPQQEVLAHGAIGAFWTHGGWNGTMESVCEGVPMIFSDFGLDQPLNARYMSDVLKVGVYLENGWDPGDIAAAIRRVMVDEAGEVMRQNARVLKQKADRSIMKGGSSYESLESLVSYISSL

SEQ ID NO: 187 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEEFRKELEKLMAASESDETVSCLITDALWYFAQSVADSLNLKRLVLQTGSLFNFHCLVSLPKFDELGYLDPDDKTRLDEPAEGFPMLKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDEKTFLEIARGLVLSKQSFLWLVRPGFVKGKTWIEPLPEGFLGEKGRVVKSAPQQEVLAHGAIGAFWTHGGWNGTMEAVCEGVPMIFSDFGLDQPLNARYMSDVLKVGVYLENGWNPEEIAEAIRRVMVDESGEYIRENAKVLKQKADRSIMKGGSSYESLESLVSYISSL

SEQ ID NO: 188 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFKEALRLLMLASESDEEVSCLITDALWYFAQSVADSLNLKRLVLQTGSLFNFHCLVSLPKFDELGYLDPDDKTNLDEPAVGFPMLKVKDIKSAYSHWQESKPILDKMVKQTRASSGVIWNSFKELEESELETVQREIPAPSFLIPLPKHYRASSTSLLDTDRSVFQWLDQQPPSSVLYVGFGSQSEVDPEDFLKIAQGLVDSGQSFLWLVRPGFVKGSEWIEPLPDGFLGEKGRIVKSAPQQEVLAHGAIGAFWTHGGWNGTMESVCEGVPMIFSDFGLDQPLNARYMSDVLHVGVYLENGWNPEDIAAAIRKVMTDPEGEKMRQNARVLKQKADRSIMKGGSSYESLESLVSYISSL

SEQ ID NO: 189 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALEELINASESDEEVSCLITDALWYFAQSVADSLNLKRLVLQTGSLFNFHCLVSLPKFDELGYLDPDDKTRLDEPVPGFPRLKVKDIKSAYSHWQESKPILEKMVKQTRASSGVIWNSFKELEESELETVQREIPAPSFLIPLPKHYRASSTSLLDTDRSVFQWLDTQPPGSVLYVGFGSQSEVDEATFLEIARGLVDSGQSFLWLVRPGFVKGKTWIEPLPDGFLGEKGRVVKSAPQQEVLAHPAIGAFWTHGGWNGTMEAVCEGVPMIFSDFGLDQPLNARYMSDVLKVGVYLENGWDRGEIADAIRYVMVDPEGEPIRENARVLKQKADRSIMKGGSSYESLESLVSYISSL

SEQ ID NO: 190 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFRKELEELILASESDEEVSCLITDALWYFAQDVADSLNLKRLVLQTGSLFNFHCLVSLPKFDELGYLDPDDKTRLEEPAEGFPYLKVKDIKSAYSHWQESKPILMKMVKQTRASSGVIWNSFKELEESELETVQREIPAPSFLIPLPKHYRASSTSLLDTDRSVFQWLDTQPPGSVLYVGFGSQSEVDPETFLEIVDGLVDSKLPFLWLVRPGFVKGKTWIEPLPDGYLGEKGRVVKSAPQQEVLAHPAIGAFWTHGGWNGTMESVCEGVPMIFSDFGLDQPLNARYMSEVLKVGVYLENGWNRDEIAEAIRRVMVDEEGEPIRENARVLKQKADRSIMKGGSSYESLESLVSYISSL

SEQ ID NO: 191 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADVFRKLLEELMKASESDEEVSCLITDALWYFAQEVADSLNLKRLVLQTGSLFNFHCLVSLPKFDELGYLDPDNKENLEEPAEGFPELKVKDIKSAYSHWQESKPILMKMVKQTRASSGVIWNSFKELEESELETVQREIPAPSFLIPLPKHYRASSTSLLDTDRSVFQWLDTQPPRSVLYVGFGSQSEVDPETFLEIAEGLVASGQPFLWLVRPGFVKGSTWIEPLPEGFLGEKGRVVKSAPQQEVLAHPAIGAFWTHGGWNGTMESVCEGVPMIFSDFGLDQPLNARYMSDVLKVGVYLDNGWDRDAIAAAIRRVMVDPEGEPMRENARVLKQKADRSIMKGGSSYESLESLVSYISSL

SEQ ID NO: 192 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFRKELEDLILASESDEEVSCLITDALWYFAQDVADSLNLRRLVLQTGSLFNFHCLVSLPKFDELGYLDPDNKENLEEPVEGFPYLKVKDIKRAYSHWQESKPILEKMVKQTKASSGVIWNSFKELEESELETVWREIPAPSFLIPLPKHYRASSTSLLDTDRSVFQWLDQQPPGSVLYVGFGSQSEVDPKDFLTIAEGLVKSGLPFLWLVRPGFVKGSTWIEPLPDGFLGEKGRIVKSAPQQEVLAHPAIGAFWTHGGWNGTMESVCEGVPMIFSDFGLDQPLNARYMSDVLKVGVYLENGWDPDEIAAAIRRVMVDPEGEYIRENAKVLKQKADRSIMKGGSSYESLESLVSYISSL

SEQ ID NO: 193 - protein, 457 aa, synthetic construct
MPNTNTTVRRRRRIVLFPVPFPGHLNPMLQLANVLYSKGFSITIFHTNFNKPKTSNYPHFTFRFILDNDPQDEWLRNLPTHGPGVGARIPVINKHGADEFRKELEELMKASESDEEVSCLITDALWYFAQSVADSLNLKRLVLQTGSLFNFHCLVSLPKFDELGYLDPDNKTDLEKPAEGFPWLKVKDIKSAYSHWQESKPILDKMVKQTRASSGVIWNSFKELEESELETVQREIPAPSFLIPLPKHYRASSTSLLDTDRSVFQWLDTQPPGSVLYVGFGSQSEVDPETFLKIAEGLVASGQRFLWLVRPGFVKGSEWIEPLPDGFLGEKGRIVKSAPQQEVLAHPAIGAFWTHGGWNGTMESVCEGVPMIFSDFGLDQPLNARYMSDVLHVGVYLENGWEPGEIAAAIRYVMVDPEGEVIRQNAAVLKQKADRSIMKGGSSYESLESLVSYISSL

SEQ ID NO: 194 - protein, 457 aa, synthetic construct
MPNTNTTVRRRRRIILFPVPFPGHLNPMLQLANVLYSKGFSITIFHTNFNKPKTSNYPHFTFVTILDNDPQDEWLRNLPTHGPGVGARIPVINKHGAEQFRKLLEELMLASESDEEVSCLITDALWYFAQDVADSLNLKRLVLQTGSLFNFHCLVSLPKFDELGYLDPDDKTRLDEPAEGFPELKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDEKDFLAIAEGLVKSGIPFLWLVRPGFVKGSTWIEPLPEGFLGEKGRVVKSAPQQEVLAHPAIGAFWTHGGWNGTMEAVCEGVPMIFSDFGLDQPLNARYMSDVLHVGVYLDNGWNPEEIANAIRRVMRDPEGEPIRQNARVLKQKADRSIMKGGSSYESLESLVSYISSL

SEQ ID NO: 195 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGAEQFRKLLEELIAASESDEEVSCLITDALWYFAQDVADSLNLRRLVLQTGSLFNFHCLVSLPKFDELGYLDPDDKTRLEEPVEGFPMLKVKDIKRAYSHWQESKPILEKMVKQTKASSGVIWNSFKELEESELETVWREIPAPSFLIPLPKHYRASSTSLLDTDRSVFQWLDQQPPGSVLYVGFGSQSEVDPEDFLKIVEGLVLSKQNFLWLVRPGFVKGKTWIEPLPDGFLGEKGRIVKSAPQQEVLAHPAIGAFWTHGGWNGTMESVCEGVPMIFSDFGLDQPLNARYMSDVLKVGVYLENGWDPEEIANAIRYVMVDEEGEPIRQNARVLKQKADRSIMKGGSSYESLESLVSYISSL

SEQ ID NO: 196 - protein, 457 aa, synthetic construct
MPNTNTTVRRRRRIILFPVPFPGHLNPMLQLANVLYSKGFSITIFHTNFNKPKTSNYPHFTFIFILDNDPQDEWLRNLPTHGPGVGARIPVINKHGAEEFRKALEELIEASESDEEVSCLITDALWYFAQDVADSLNLKRLVLQTGSLFNFHCLVSLPKFDELGYLDPDDKTRLEEPAEGFPMLKVKDIKSAYSHWQESKPILDKMVKQTRASSGVIWNSFKELEESELETVQREIPAPSFLIPLPKHYRASSTSLLDTDRSVFQWLDQQPPSSVLYVGFGSQSEVDPETFLKIVQGLVDSGLPFLWLVRPGFVKGKEWIEPLPDGFLGEKGRIVKSAPQQEVLAHPAIGAFWTHGGWNGTMESVCEGVPMIFSDFGLDQPLNARYMSDVLKVGVYLENGWEPGAIANAIRYVMVDEAGEVIRQNARVLKQKADRSIMKGGSSYESLESLVSYISSL

SEQ ID NO: 197 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFRKELELLISASESDEEVSCLITDALWYFAQSVADSLNLKRLVLQTGSLFNFHCLVSLPKFDELGYLDPDNKTDLEKPAEGFPYLKVKDIKSAYSHWQESKPILMKMVKQTRASSGVIWNSFKELEESELETVQREIPAPSFLIPLPKHYRASSTSLLDTDRSVFQWLDQQPPGSVLYVGFGSQSEVDPEDFLTIAQGLVDSGQSFLWLVRPGFVKGSTWIEPLPDGFLGEKGRVVKSAPQQEVLAHGAIGAFWTHGGWNGTMESVCEGVPMIFSDFGLDQPLNARYMSEVLKVGVYLENGWEPAAIAAAIRYVMTDPAGEPIRENAKVLKQKADRSIMKGGSSYESLESLVSYISSL

SEQ ID NO: 198 - protein, 457 aa, synthetic construct
MPNTNTTVRRRRRIILFPVPFPGHWNPMLQLANVLYSKGFSITIFHTNFNKPKTSNYPHFTFVFILDNDPQDEWLRNLPTHGPGVGARIPVINKHGAEEFRKALEELIAASESDEEVSCLITDALWYFAQEVADSLNLKRLVLQTGSLFNFHCLVSLPKFDELGYLDPDNKTDLEKPVEGFPYLKVKDIKSAYSHWQESKPILMKMVKQTRASSGVIWNSFKELEESELETVQREIPAPSFLIPLPKHYRASSTSLLDTDRSVFQWLDTQPPGSVLYVGFGSQSEVDPETFLKIAEGLVLSGQNFLWLVRPGFVKGSTWIEPLPEGFLGEKGRVVKSAPQQEVLAHPAIGAFWTHGGWNGTMESVCEGVPMIFSDFGLDQPLNARYMSDVLKVGVYLENGWDPEEIAAAIRRVMVDEEGKPIRENAKVLKQKADRSIMKGGSSYESLESLVSYISSL

SEQ ID NO: 199 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGAERFRKELELLMAASESDEEVSCLITDALWYFAQDVADSLNLKRLVLQTGSLFNFHCLVSLPKFDELGYLDPDDKTRLEEPAEGFPMLKVKDIKSAYSHWQESKPILMKMVKQTRASSGVIWNSFKELEESELETVQREIPAPSFLIPLPKHYRASSTSLLDTDRSVFQWLDTQPPESVLYVGFGSQSEVDPETFLEIARGLVDSGIPFLWLVRPGFVKGSTWIEPLPDGFLGEKGRVVKSAPQQEVLAHPAIGAFWTHGGWNGTMESVCEGVPMIFSDFGLDQPLNARYMSEVLKVGVYLDNGWDPAEIAAAIRYVMRDEAGEPIRQNAKVLKQKADRSIMKGGSSYESLESLVSYISSL

SEQ ID NO: 200 - protein, 457 aa, synthetic construct
MPNTNTTVRRRRRIILFPVPFPGHWNPMLQLANVLYSKGFSITIFHTNFNKPKTSNYPHFTFIFILDNDPQDEWLRNLPTHGPGVGARIPVINKHGAEEFRKTLEELMAASESDEEVSCLITDALWYFAQDVADSLNLKRLVLQTGSLFNFHCLVSLPKFDELGYLDPDNKENLEEPVEGFPYLKVKDIKSAYSHWQESKPILMKMVKQTRASSGVIWNSFKELEESELETVQREIPAPSFLIPLPKHYRASSTSLLDTDRSVFQWLDTQPPGSVLYVGFGSQSEVDEKDFLAIVDGLVKSGQPFLWLVRPGFVKGKTWIEPLPEGYLGEKGRVVKSAPQQEVLAHPAIGAFWTHGGWNGTMESVCEGVPMIFSDFGLDQPLNARYMSDVLKVGVYLENGWDPDEIANAIRYVMVDEEGQPIRDNAAVLKQKADRSIMKGGSSYESLESLVSYISSL

SEQ ID NO: 201 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGAEEFRKALENLINASESDEEVSCLITDALWYFAQPVADSLNLKRLVLQTGSLFNFHCLVSLPKFDELGYLDPDNKENLDEPVEGFPELKVKDIKSAYSHWQESKPILEKMVKQTRASSGVIWNSFKELEESELETVQREIPAPSFLIPLPKHYRASSTSLLDTDRSVFQWLDQQPPGSVLYVGFGSQSEVDPETFLTIVEGLVKSGLPFLWLVRPGFVKGSTWIEPLPEGFLGEKGRVVKSAPQQEVLAHPAIGAFWTHGGWNGTMEAVCEGVPMIFSDFGLDQPLNARYMSDVLKVGVYLENGWDPDAIAAAIRLVMRDEAGEPIRENARVLKQKADRSIMKGGSSYESLESLVSYISSL

SEQ ID NO: 202 - protein, 457 aa, synthetic construct
MPNTNTTVRRRRRIILFPVPFPGHLNPMLQLANVLYSKGFSITIFHTNFNKPKTSNYPHFTFRTILDNDPQDEWLRNLPTHGPGVGARIPVINKHGADQFRKLLEELIAASESDEEVSCLITDALWYFAQDVADSLNLKRLVLQTGSLFNFHCLVSLPKFDELGYLDPDDKTDLDKPAEGFPELKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDEKTFLEIARGLVASGLPFLWLVRPGFVKGKTWIEPLPDGFLGEKGRVVKSAPQQEVLAHPAIGAFWTHGGWNGTMEAVCEGVPMIFSDFGLDQPLNARYMSDVLKVGVYLDNGWDADEIAAAIRRVMVDEAGKPMRENARVLKQKADRSIMKGGSSYESLESLVSYISSL

SEQ ID NO: 203 - protein, 457 aa, synthetic construct
MPNTNTTVRRRRRIVLFPVPFPGHWNPMLQLANVLYSKGFSITIFHTNFNKPKTSNYPHFTFRFILDNDPQDEWLRNLPTHGPGVGARIPVINKHGADRFRKELEELMNASESDEEVSCLITDALWYFAQDVADSLNLKRLVLQTGSLFNFHCLVSLPKFDELGYLDPDDKTDLEKPAVGFPELKVKDIKSAYSHWQESKPILMKMVKQTRASSGVIWNSFKELEESELETVQREIPAPSFLIPLPKHYRASSTSLLDTDRSVFQWLDQQPPESVLYVGFGSQSEVDPEDFLRIVEGLVLSGQPFLWLVRPGFVKGSTWIEPLPEGFLGEKGRVVKSAPQQEVLAHPAIGAFWTHGGWNGTMESVCEGVPMIFSDFGLDQPLNARYMSEVLKVGVYLENGWNPEEIANAIRYVMVDEAGKPIRENARVLKQKADRSIMKGGSSYESLESLVSYISSL

SEQ ID NO: 204 - protein, 457 aa, synthetic construct
MPNTNTTVRRRRRIILFPVPFPGHLNPMLQLANVLYSKGFSITIFHTNFNKPKTSNYPHFTFKFILDNDPQDEWLRNLPTHGPGVGARIPVINKHGAEQFRKLLEELIAASESDETVSCLITDALWYFAQDVADSLNLRRLVLQTGSLFNFHCLVSLPKFDELGYLDPDNKERLEEPAEGFPMLKVKDIKRAYSHWQESKPILEKMVKQTKASSGVIWNSFKELEESELETVWREIPAPSFLIPLPKHYRASSTSLLDTDRSVFQWLDTQPPESVLYVGFGSQSEVDPEDFLKIVEGLVASGIPFLWLVRPGFVKGSTWIEPIPEGFLGEKGRIVKSAPQQEVLAHPAIGAFWTHGGWNGTMESVCEGVPMIFSDFGLDQPLNARYMSEKLKVGVYLDDGWDPEAIAAAIRYVMRDEAGKPIRENAKVLKQKADRSIMKGGSSYESLESLVSYISSL

SEQ ID NO: 205 - protein, 457 aa, synthetic construct
MPNTNTTVRRRRRIILFPVPFPGHWNPMLQLANVLYSKGFSITIFHTNFNKPKTSNYPHFTFVFILDNDPQDEWLRNLPTHGPGVGARIPVINKHGAKRFEELLKKLIEASESDEEVSCLITDALWYFAQDVADSLNLKRLVLQTGSLFNFHCLVSLPKFDELGYLDPDNKERLEEPAEGFPWLKVKDIKSAYSHWQESKPILMKMVKQTRASSGVIWNSFKELEESELETVQREIPAPSFLIPLPKHYRASSTSLLDTDRSVFQWLDTQPPESVLYVGFGSQSEVDPEDFLKIVEGLVKSGIPFLWLVRPGFVKGSTWIEPIPEGFLGEKGRVVKSAPQQEVLAHPAIGAFWTHGGWNGTMESVCEGVPMIFSDFGLDQPLNARYMSDVLKVGVYLDNGWDPDEIAAAIRYVMRDEAGQPIRDNAKVLKQKADRSIMKGGSSYESLESLVSYISSL

SEQ ID NO: 206 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAERFKALLEEEIAASESDEEISCLITDALWYFAQEVADSLNLRRLVLQTGSLFNFHCLVSLPKFDELGYLDPDDKTKLDEPAEGFPMLKVKDIKRAYSHWQESKPILEKMVKQTKASSGVIWNSFKELEESELETVWREIPAPSFLIPLPKHYRASSTSLLDTDRSVFQWLDTQAPESVLYVGFGSQSEVDVDDFLTIMEGLVKSGISFLWLVRPGFVKGQTWIEPIPEGFLGEKGRIVKSAPQQEVLAHGAIGAFWTHGGWNGTMESVCEGVPMIFSDFGLDQPLNARYMSDKLKVGVYLENGWDPDAIAAAIRYVMKDEAGKPIRENAKVLKQKADRSIMKGGSSYESLESLVSYISSL

SEQ ID NO: 207 - protein, 457 aa, synthetic construct
MPNTNTTVRRRRRIILFPVPFPGHLNPMLQLANVLYSKGFSITILHTNFNKPKTSNYPHFTFIEILDNDPQDEWLRNLPTHGPGVGARIPVINKHGADEFRKTLEALMAASESDEEVSCLITDALWYFAQDVADSLNLKRLVLQTGSLFNFHCLVSLPKFEELGYLDPSDKTRLEEPAEGFPWLKVKDIKSAYSHWQESKPILDKMVKQTRASSGVIWNSFKELEESELETVQREIPAPSFLIPLPKHYRASSTSLLDTDRSVFDWLDTQPPSSVLYVGFGSQSEVSPEDFLKIAEGLVLSKQPFLWLVRPGFVKGSEWIEPLPEGFLGEKGRIVKSAPQQEVLAHPAIGAFWTHGGWNGTMESVCEGVPMIFSDFGLDQPLNARYMSDVLHVGVYLDNGWEPEEIANAIRRVMVDEEGEVIRENARVLKQKADRSIMKGGSSYESLESLVSYISSL

SEQ ID NO: 208 - protein, 457 aa, synthetic construct
MPNTNTTVRRRRRIILFPVPFPGHLNPMLQLANVLYSKGFSITILHTNFNKPKTSNYPHFTFRTILDNDPQDEWLRNLPTHGPGVGARIPVINKHGADEFRKILEELMAASESDEEVSCLITDALWYFAQDVADSLNLRRLVLQTGSLFNFHCLVSLPKFEELGYLDPNDKTNLEEPAEGFPMLKVKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVDPKDFLEIVDGLVASKQPFLWLVRPGFVKGYEWIEPLPEGYLGEKGRIVKSAPQQEVLAHPAIGAFWTHGGWNGTMESVCEGVPMIFSDFGLDQPLNARYMSEVLKVGVYLDNGWDREEIAAAIRYVMVDEAGEPIRQNARVLKQKADRSIMKGGSSYESLESLVSYISSL

SEQ ID NO: 209 - protein, 457 aa, synthetic construct
MPNTNTTVRRRRRIILFPVPFPGHLNPMLQLANVLYSKGFSITIFHTNFNKPKTSNYPHFTFRTILDNDPQDEWLRNLPTHGPGVGARIPVINKHGADQFRKELELLMLASESDEEVSCLITDALWYFAQDVADSLNLKRLVLQTGSLFNFHCLVSLPKFEELGYLDPNDKTNLDEPAEGFPMLKVKDIKSAYSHWQESKPILEKMVKQTRASSGVIWNSFKELEESELETVQREIPAPSFLIPLPKHYRASSTSLLDTDRSVFDWLDTQPPRSVLYVGFGSQSEVTPEEFLKIADGLVASGISFLWLVRPGFVKGKEWIEPLPEGYLGEKGRVVKSAPQQEVLAHPAIGAFWTHGGWNGTMEAVCEGVPMIFSDFGLDQPLNARYMSDVLKVGVYLENGWDPAEIAAAIRRVMVDPEGEPIRQNAEVLKQKADRSIMKGGSSYESLESLVSYISSL

SEQ ID NO: 210 - protein, 457 aa, synthetic construct
MPNTNTTVRRRRRIVLFPVPFPGHLNPMLQLANVLYSKGFSITILHTNFNKPKTSNYPHFTFIFTLDNDPQDEWLRNLPTHGPGVGARIPVINKHGADEFRKTLESLINASESDETVSCLITDALWYFAQSVADSLNLKRLVLQTGSLFNFHCLVSLPKFDALGYLDPDDKTRLDEPVEGFPMLKVKDIKSAYSHWQESKPILDKMVKQTRASSGVIWNSFKELEESELETVQREIPAPSFLIPLPKHYRASSTSLLDTDRSVFAWLDTQPPESVLYVGFGSQSEVDEKTFLEIAEGLVKSGLPFLWLVRPGFVKGKEWIEPLPDGFLGEKGRIVKSAPQQEVLAHPAIGAFWTHGGWNGTMESVCEGVPMIFSDFGLDQPLNARYMSDVLHVGVYLDNGWEPGEIAAAIRRVMVDEEGEKIRENARVLKQKADRSIMKGGSSYESLESLVSYISSL

SEQ ID NO: 211 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGAEEFRKALEKLMAASESDEVVSCLITDALWYFAQSVADSLNLKRLVLQTGSLFNFHCLVSLPKFEELGYLDPEDKTGLEEPVEGFPELKVKDIKSAYSHWQESKPILDKMVKQTRASSGVIWNSFKELEESELETVQREIPAPSFLIPLPKHYRASSTSLLDTDRSVFAWLDQQPPESVLYVGFGSQSEVDPADFLTIAEGLVRSGQPFLWLVRPGFVKGSEWIEPLPDGFLGEKGRIVKSAPQQEVLAHPAIGAFWTHGGWNGTMESVCEGVPMIFSDFGLDQPLNARYMADVKKVGVYLDNGWDPDEIAAAIRRVMVDPAGEAMRENAKVLKQKADRSIMKGGSSYESLESLVSYISSL

SEQ ID NO: 212 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALEELMAASESDETVSCLITDALWYFAQDVADSLNLKRLVLQTGSLFNFHCLVSLPKFDELGYLDPNDKTRLDEPVEGFPELKVKDIKSAYSHWQESKPILDKMVKQTRASSGVIWNSFKELEESELETVQREIPAPSFLIPLPKHYRASSTSLLDTDRSVFDWLDQQPPGSVLYVGFGSQSEVDPETFLEIADGLVASKQSFLWLVRPGFVKGSEWIEPLPDGYLGEKGRIVKSAPQQEVLAHPAIGAFWTHGGWNGTMESVCEGVPMIFSDFGLDQPLNARYMSDVLKVGVYLDNGWDRDAIAAAIRYVMTDKAGEVIRNNAKVLKQKADRSIMKGGSSYESLESLVSYISSL

SEQ ID NO: 213 - protein, 457 aa, synthetic construct
MPNTNTTVRRRRRIILFPVPFPGHLNPMLQLANVLYSKGFSITILHTNFNKPKTSNYPHFTFIFTLDNDPQDEWLRNLPTHGPGVGARIPVINKHGAEEFRKTLEELMLASESDEKVSCLITDALWYFAQEVADSLNLKRLVLQTGSLFNFHCLVSLPKFDELGYLDPENKERLEEPVVGFPELKVKDIKSAYSHWQESKPILDKMVKQTRASSGVIWNSFKELEESELETVQREIPAPSFLIPLPKHYRASSTSLLDTDRSVFAWLDTQPPGSVLYVGFGSQSEVDEKDFLEIAQGLVDSGQPFLWLVRPGFVKGKEWIEPLPDGFLGEKGRIVKSAPQQEVLAHGAIGAFWTHGGWNGTMESVCEGVPMIFSDFGLDQPLNARYMSDVLKVGVYLENGWDPKEIANAIRTVMVDEKGEAIRQNARVLKQKADRSIMKGGSSYESLESLVSYISSL

SEQ ID NO: 214 - protein, 457 aa, synthetic construct
MPNTNTTVRRRRRIILFPVPFPGHWNPMLQLANVLYSKGFSITILHTNFNKPKTSNYPHFTFRFILDNDPQDEWLRNLPTHGPGVGARIPVINKHGAEEFRKTLEELMLASESDETVSCLITDALWYFAQDVADSLNLKRLVLQTGSLFNFHCLVSLPKFDELGYLDPNNKERLEEPVPGFPYLKVKDIKSAYSHWQESKPILMKMVKQTRASSGVIWNSFKELEESELETVQREIPAPSFLIPLPKHYRASSTSLLDTDRSVFAWLDQQPPGSVLYVGFGSQSEVDEADFLTIAEGLVASGQNFLWLVRPGFVKGKEWIEPLPEGFLGEKGRVVKSAPQQEVLAHPAIGAFWTHGGWNGTMESVCEGVPMIFSDFGLDQPLNARYMSDVLKVGVYLENGWNADDIAAAIRRVMTDPAGAAMRENARVLKQKADRSIMKGGSSYESLESLVSYISSL

SEQ ID NO: 215 - protein, 457 aa, synthetic construct
MPNTNTTVRRRRRIILFPVPFPGHLNPMLQLANVLYSKGFSITILHTNFNKPKTSNYPHFTFVTILDNDPQDEWLRNLPTHGPGVGARIPVINKHGAERFRKILEELMAASESDEEISCLITDALWYFAQEVADSLNLRRLVLQTGSLFNFHCLVSLPKFEELGYLDPSDKTNLEEPAEGFPMLKVKDIKRAYSHWQESKPILEKMVKQTKASSGVIWNSFKELEESELETVWREIPAPSFLIPLPKHYRASSTSLLDTDRSVFDWLDQQPPESVLYVGFGSQSEVTPEEFLTIAEGLVRSGIPFLWLVRPGFVKGQAWIEVLPDGFLGEKGRIVKSAPQQEVLAHGAIGAFWTHGGWNGTMESVCEGVPMIFSDFGLDQPLNARYMSEKLKIGVYLDNGWNPEEIANAIRLVMRDEKGKPMRENARVLKQKADRSIMKGGSSYESLESLVSYISSL

SEQ ID NO: 216 - protein, 457 aa, synthetic construct
MPNTNTTVRRRRRIVLFPVPFPGHLNPMLQLANVLYSKGFSITIFHTEFNKPKTSNYPHFTFVTILDNDPQDEWLRNLPTHGPGVGARIPVINKHGADRFRKELEKLIAASESDEEVSCLITDALWYFAQDVADSLNLKRLVLQTGSLFNFHCLVSLPKFEELGYLDPSDKTDLDKPAPGFPELKVKDIKSAYSHWQESKPILEKMVKQTRASSGVIWNSFKELEESELETVQREIPAPSFLIPLPKHYRASSTSLLDTDRSVFAWLDTQPPRSVLYVGFGSQSEVTPEEFLTIVQGLVDSKIPFLWLVRPGFVKGSAWIEPIPEGFLGELGRVVKSAPQQEVLAHPAIGAFWTHGGWNGTMEAVCEGVPMIFSDFGLDQPLNARYMSDVLKIGVYLDNGWDPEAIANAIRRVMLDPEGEPIRENARVLKQKADRSIMKGGSSYESLESLVSYISSL

SEQ ID NO: 217 - protein, 457 aa, synthetic construct
MPNTNTTVRRRRRIILFPVPFPGHLNPMLQLANVLYSKGFSITILHTNFNKPKTSNYPHFTFIFTLDNDPQDEWLRNLPTHGPGVGARIPVINKHGADEFLKTLKLLMEASESDEEVSCLITDALWYFAQDVADSLNLKRLVLQTGSLFNFHCLVSLPKFDELGYLDPSNTTRLDEPVPGFPMLKVKDIKSAYSHWQESKPILEKMVKQTRASSGVIWNSFKELEESELETVQREIPAPSFLIPLPKHYRASSTSLLDTDRSVFDWLDTQPPGSVLYVGFGSQSEVDPETFLKIARGLVLSKQSFLWLVRPGFVKGSAWIEPLPEGFLGERGRVVKSAPQQEVLAHPAIGAFWTHGGWNGTMEAVCEGVPMIFSDFGLDQPLNARYMSDVLKVGVYLDNGWDPEEIAAAIRYVMVDEAGKPIRDNAAVLKQKADRSIMKGGSSYESLESLVSYISSL

SEQ ID NO: 218 - protein, 457 aa, synthetic construct
MPNTNTTVRRHRRIVLFPVPFPGHLNPMLQLANVLYSKGFSITIIHTNFNKPKTSNYPHFTFRFILDNDPQDEWLRNLPTHGPGVGARIPVINKHGADELRKLLEELMAASESDEEISCLITDALWYFAQEVADSLNLRRLVLQTGSLFNFHCLVSLPKFDELGYLDPDDKTDLEKPVEGFPYLKVKDIKRAYSHWQESKPILEKMVKQTKASSGVIWNSFKELEESELETVWREIPAPSFLIPLPKHYRASSTSLLDTDRSVFQWLDTQPPESVLYVGFGSQSEVDPETFLKIAEGLVKSGLNFLWLVRPGFVKGYDWIEPLPDGFLGEKGRIVKSAPQQEVLAHPAIGAFWTHGGWNGTMESVCEGVPMIFSDFGLDQPLNARYMSDVLKVGVYLDNGWEPDAIAAAIRHVMRDESGAYIRENARVLKQKADRSIMKGGSSYESLESLVSYISSL

SEQ ID NO: 219 - protein, 457 aa, synthetic construct
MPNTNTTVRRRRRIILFPVPFPGHLNPMLQLANVLYSKGFSITIFHTNFNKPKTSNYPHFTFVEILDNDPQDEWLRNLPTHGPGVGARIPVINKHGAERFKKELEKLIEASESDEEVSCLITDALWYFAQDVADSLNLRRLVLQTGSLFNFHCLVSLPKFEELGYLDPDDTTNLDEPAVGFPMLKVKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVTPKEFLEIVRGLVKSGIPFLWLVRPGFVKGYDWIEPIPEGFLGEKGRIVKSAPQQEVLAHPAIGAFWTHGGWNGTMESVCEGVPMIFSDFGLDQPLNARYMSEKLHVGVYLDNGWNPDEIADAIRYVMRDPEGEPIRENAKVLKQKADRSIMKGGSSYESLESLVSYISSL

SEQ ID NO: 220 - protein, 457 aa, synthetic construct
MPNTNTTVRRRRRIILFPVPFPGHLNPMLQLANVLYSKGFSITIFHTEFNKPKTSNYPHFTFVTILDNDPQDEWLRNLPTHGPGVGARIPVINKHGADQFRKLLEELIAASESDETVSCLITDALWYFAQDVADSLNLKRLVLQTGSLFNFHCLVSLPKFEELGYLDPSNTTGLDEPVEGFPELLVKDIKSAYSHWQESKPILEKMVKQTRASSGVIWNSFKELEESELETVQREIPAPSFLIPLPKHYRASSTSLLDTDRSVFAWLDTQPPESVLYVGFGSQSEVTPEDFLTIAEGLVASGLPFLWLVRPGFVKGYDWIEPLPEGFLGERGRVVKSAPQQEVLAHGAIGAFWTHGGWNGTMEAVCEGVPMIFSDFGLDQPLNARYMSDVKKVGVYLRNGWDPGEIAAAIRYVMRDEEGKYIRENAKVLKQKADRSIMKGGSSYESLESLVSYISSL

SEQ ID NO: 221 - protein, 457 aa, synthetic construct
MPNTNTTVRRRRRIILFPVPFPGHLNPMLQLANVLYSKGFSITIFHTNFNKPKTSNYPHFTFVEILDNDPQDEWLRNLPTHGPGVGARIPVINKHGAEEFRKALEELIKASESDEVVSCLITDALWYFAQDVADSLNLKRLVLQTGSLFNFHCLVSLPKFEKLGYLDPSDKTGLEEPVVGFPELKVKDIKSAYSHWQESKPILDKMVKQTRASSGVIWNSFKELEESELETVQREIPAPSFLIPLPKHYRASSTSLLDTDRSVFAWLDTQPPESVLYVGFGSQSEVSPEEFLEIVDGLVASGISFLWLVRPGFVKGKEWIEPLPEGYLGEKGRIVKSAPQQEVLAHGAIGAFWTHGGWNGTMESVCEGVPMIFSDFGLDQPLNARYMSDVLKIGVYLDNGWNRDDIAAAIRYVMEDEAGKEIRENARVLKQKADRSIMKGGSSYESLESLVSYISSL

SEQ ID NO: 222 - protein, 457 aa, synthetic construct
MPNTNTTVRRRRRIILFPVPFPGHLNPMLQLANVLYSKGFSITILHTNFNKPKTSNYPHFTFVYILDNDPQDEWLRNLPTHGPGVGARIPVINKHGAEEFRKTLEEIILASESDEEVSCLITDALWYFAQDVADSLNLKRLVLQTGSLFNFHCLVSLPKFEELGYLDPSDTTGLEEPVEGFPELKVKDIKSAYSHWQESKPILDKMVKQTRASSGVIWNSFKELEESELETVQREIPAPSFLIPLPKHYRASSTSLLDTDRSVFEWLDTQPPRSVLYVGFGSQSEVTPKDFLAIARGLVDSGIPFLWLVRPGFVKGSEWIEPLPEGFLGERGRIVKSAPQQEVLAHPAIGAFWTHGGWNGTMESVCEGVPMIFSDFGLDQPLNARYMSDVLKIGVYLDDGWDPAAIAAAIRRVMRDPEGEVIRQNAEVLKQKADRSIMKGGSSYESLESLVSYISSL

SEQ ID NO: 223 - protein, 457 aa, synthetic construct
MPNTNTTVRRRRRIVLFPVPFPGHLNPMLQLANVLYSKGFSITIFHTNFNKPKTSNYPHFTFVEILDNDPQDEWLRNLPTHGPGVGARIPVINKHGAEQFKKLLKLLIEASESDETLSCLITDALWYFAQSVADSLNLKRLVLQTGSLFNFHCLVSLPLFDKLGYLDPDNKTNLDEPAVGFPELKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDEEDFLTIADGLVASKQPFLWLVRPGFVKGHTWIEPLPEGYLGEKGRVVKSAPQQEVLAHPAIGAFWTHGGWNGTMEAVCEGVPMIFSDFGLDQPLNARYMSDVLKVGVYLDNGWNRGDIAAAIRYVMVDPEGEYIRENAKVLKQKADRSIMKGGSSYESLESLVSYISSL

SEQ ID NO: 224 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAAEFKAALEELIAASESDETVSCLITDALWYFAQSVADSLGLKRLVLQTGSLFNFHCLVSLPKFDALGYLDPDNKENLDEPAEGFPMLKVKDIKSAYSHWQESKPILDKMVKQTRASSGVIWNSFKELEESELETVQREIPAPSFLIPLPKHYRASSTSLLDTDRSVFQWLDTQPPGSVLYVGFGSQSEVDEATFLAIADGLVASGLSFLWLVRPGFVKGKEWIEPLPDGYLGERGRIVKSAPQQEVLAHGAIGAFWTHGGWNGTMESVCEGVPMIFSDFGLDQPLNARYMSDVLEVGVYLENGWDPAAIAAAIRRVMRAPEGEKIRENAKVLKQKADRSIMKGGSSYESLESLVSYISSL

SEQ ID NO: 225 - protein, 457 aa, synthetic construct
MPNTNTTVRRRRRIILFPVPFPGHLNPMLQLANVLYSKGFSITIIHTNFNKPKTSNYPHFTFRTILDNDPQDEWLRNLPTHGPGVGARIPVINKHGAEQLKKLLEEIMEASESDETISCLITDALWYFAEDVAKSLNLRRLVLQTGSLFNFHCLVSLPKFEKLGYLDPSDTTGLEEPVEGFPMLKRKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVSEADFLAIADGLVASGIPFLWLVRPGFVKGKAWIEPLPEGYLGERGRIVKSAPQQEVLAHPAIGAFWTHGGWNGTMESVCEGVPMIFSDFGLDQPLNARYMSDVKKIGVYLDDGWDPAAIAAAIRLVMRDPAGEPIRENAKVLKQKADRSIMKGGSSYESLESLVSYISSL

SEQ ID NO: 226 - protein, 457 aa, synthetic construct
MPNTNTTVRRRRRIVLFPVPFPGHWNPMLQLANVLYSKGFSITIFHTNFNKPKTSNYPHFTFVTILDNDPQDEWLRNLPTHGPGVGARIPVINKHGAERFEKLLKELIAASESDEVVSCLITDALWYFALDVAKKLNLKRLVLQTGSLFNFHCLVSLPKFDELGYLDPSDKTRLEEPAVGFPELKVKDIKSAYSHWQESKPILMKMVKQTRASSGVIWNSFKELEESELETVQREIPAPSFLIPLPKHYRASSTSLLDTDRSVFAWLDTQPPESVLYVGFGSQSEVSPEDFLTIAEGLVASGQPFLWLVRPGFVKGYDWIEPLPDGFLGEKGRVVKSAPQQEVLAHPAIGAFWTHGGWNGTMESVCEGVPMIFSDFGLDQPLNARYMSDVLKIGVYLDNGWNPADIANAIRYVMTDAAGQPIRDNAKVLKQKADRSIMKGGSSYESLESLVSYISSL

SEQ ID NO: 227 - protein, 457 aa, synthetic construct
MPNTNTTVRRRRRIVLFPVPFPGHLNPMLQLANVLYSKGFSITILHTNFNKPKTSNYPHFTFIEILDNDPQDEWLRNLPTHGPGVGARIPVINKHGAEELEKTLKELIEASESDEVISCLITDALWYFAQEVADSLNLKRLVLQTGSLFNFHCLVSLPLFEKLGYLDPSNKTDLEKPVPGFPWLKVKDIKSAYSHWQESKPILDKMVKQTRASSGVIWNSFKELEESELETVQREIPAPSFLIPLPKHYRASSTSLLDTDRSVFDWLDTQPPESVLYVGFGSQSEVTPEEFLTIAEGLVASGIPFLWLVRPGFVKGAEWIEPLPEGFLGERGRIVKSAPQQEVLAHPAIGAFWTHGGWNGTMESVCEGVPMIFSDFGLDQPLNARYMSDVLKIGVYLDNGWDPAAIAEAIRLVMRDPAGEAIRENAKVLKQKADRSIMKGGSSYESLESLVSYISSL

SEQ ID NO: 228 - protein, 457 aa, synthetic construct
MPNTNTTVRRRRRIILFPVPFPGHLNPMLQLANVLYSKGFSITIIHTEFNKPKTSNYPHFTFRTILDNDPQDEWLRNLPTHGPGVGARIPVINKHGAEQLEKILKEIIEASESDETVSCLITDALWYFAQEVADKLNLKRLVLQTGSLFNFHCLVSLPKFEELGYLDPNDTTRLDEPAPGFPELKVKDIKSAYSHWQESKPILEKMVKQTRASSGVIWNSFKELEESELETVQREIPAPSFLIPLPKHYRASSTSLLDTDRSVFAWLDTQPPESVLYVGFGSQSEVTEEEFLTIVEGLVKSGIPFLWLVRPGFVKGKEWIEPIPEGFLGEKGRVVKSAPQQEVLAHPAIGAFWTHGGWNGTMEAVCEGVPMIFSDFGLDQPLNARYMSDVLKIGVYLDNGWDPDEIAAAIRYVMRDPEGEPIRENAKVLKQKADRSIMKGGSSYESLESLVSYISSL

SEQ ID NO: 229 - protein, 457 aa, synthetic construct
MPNTNTTVRRRRRIILFPVPFPGHWNPMLQLANVLYSKGFSITIFHTNFNKPKTSNYPHFTFRTILDNDPQDEWLRNLPTHGPGVGARIPVINKHGADRFRKLLQELIEASESDEVISCLITDALWYFAQDVADSLNLKRLVLQTGSLFNFHCLVSLPKFDELGYLDPSNTTDLEKPAPGFPMLKVKDIKSAYSHWQESKPILMKMVKQTRASSGVIWNSFKELEESELETVQREIPAPSFLIPLPKHYRASSTSLLDTDRSVFEWLDTQPPESVLYVGFGSQSEVTPEEFLEIARGLVASGIPFLWLVRPGFVKGHAWIEPLPEGFLGERGRVVKSAPQQEVLAHPAIGAFWTHGGWNGTMESVCEGVPMIFSDFGLDQPLNARYMAEVKKIGVYLDDGWDPGRIAAAIRRVMRDPEGEPMRENAKVLKQKADRSIMKGGSSYESLESLVSYISSL

SEQ ID NO: 230 - protein, 457 aa, synthetic construct
MPNTNTTVRRRRRIVLFPVPFPGHWNPMLQLANVLYSKGFSITIFHTNFNKPKTSNYPHFTFVTILDNDPQDEWLRNLPTHGPGVGARIPVINKHGAERFREELEKLMLASESDEVVSCLITDALWYFAQSVADSLNLKRLVLQTGSLFNFHCLVSLPKFDELGYLDPSDKTRLEEPAEGFPWLKVKDIKSAYSHWQESKPILMKMVKQTRLSSGVIWNSFKELEESELETVQREIPAPSFLIPLPKHYRASSTSLLDTDRSVFAWLDQQPPRSVLYVGFGSQSEVTPEEFLEIVDGLVASKLPFLWLVRPGFVKGSEWIEPIPEGYLGERGRVVKSAPQQEVLAHPAIGAFWTHGGWNGTMESVCEGVPMIFSDFGLDQPLNARYMSEVKHIGVYLDDGWDRDRIAAAIRYVMRDPAGEPIRQNAEVLKQKADRSIMKGGSSYESLESLVSYISSL

SEQ ID NO: 231 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELQLLIDASESDEEVSCLITDALWYFAQSVADSLNLRRLVLQTGSLFNFHCLVSLPKFDELGYLDPDDKTDLEKPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPADFLTIAQGLVDSGQSFLWLVRPGFVKGSTWIEPLPDGFLGEKGRIVKSAPQQEVLAHGAIGAFWTHGGWNGTMESVCEGVPMIFSDFGLDQPLNARYMSEVLHVGVYLENGWEPAEIAAAIRRVMVDEEGEPMRENARVLKQKADRSIMKGGSSYESLESLVSYISSL

SEQ ID NO: 232 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFREMLKLLIEASESDEEVSCLITDALWYFAQSVADSLNLRRLVLQTGSLFNFHCLVSLPKFDELGYLDPDDKSRLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLEIARGLVLSGQSFLWLVRPGFVKGSTWIEPLPDGFLGEKGRIVKSAPQQEVLAHGAIGAFWTHGGWNGTMESVCEGVPMIFSDFGLDQPLNARYMSEVLHVGVYLENGWDPEAIAAAIRLVMTDPAGEPMRQNARVLKQKADRSIMKGGSSYESLESLVSYISSL

SEQ ID NO: 233 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKELEELIAASESDEEVSCLITDALWYFAQSVADSLNLKRLVLQTGSLFNFHCLVSLPKFDELGYLDPDDKTRLDEPAEGFPMLKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDPADFLAIARGLVLSGQSFLWLVRPGFVKGSTWIEPLPEGFLGEKGRVVKSAPQQEVLAHGAIGAFWTHGGWNGTMEAVCEGVPMIFSDFGLDQPLNARYMSDVLKVGVYLENGWDPDEIAAAIRYVMVDPAGEYIRQNARVLKQKADRSIMKGGSSYESLESLVSYISSL

SEQ ID NO: 234 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFRKLLEELMAASESDEEVSCLITDALWYFAQSVADSLNLRRLVLQTGSLFNFHCLVSLPKFDELGYLDPDDKTNLEEPAEGFPMLKVKDIKRAYSHWQESKPILEKMVKQTKASSGVIWNSFKELEESELETVWREIPAPSFLIPLPKHYRASSTSLLDTDRSVFQWLDTQPPESVLYVGFGSQSEVDPEDFLKIAEGLVKSGISFLWLVRPGFVKGSTWIEPLPEGFLGEKGRIVKSAPQQEVLAHGAIGAFWTHGGWNGTMESVCEGVPMIFSDFGLDQPLNARYMSEVLKVGVYLENGWNPEEIAKAIRYVMVDPEGEPIRENAKVLKQKADRSIMKGGSSYESLESLVSYISSL

SEQ ID NO: 235 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFRKLLEELIAASESDEEVSCLITDALWYFAQSVADSLNLKRLVLQTGSLFNFHCLVSLPKFDELGYLDPDDKTRLDEPAEGFPMLKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDEKDFLTIAEGLVKSGISFLWLVRPGFVKGSTWIEPLPEGFLGEKGRVVKSAPQQEVLAHGAIGAFWTHGGWNGTMEAVCEGVPMIFSDFGLDQPLNARYMSDVLKVGVYLENGWNADEIAAAIRYVMRDEAGEPIRENARVLKQKADRSIMKGGSSYESLESLVSYISSL

SEQ ID NO: 236 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEEFRKELELLMAASESDEEVSCLITDALWYFAQSVADSLNLKRLVLQTGSLFNFHCLVSLPKFDELGYLDPDDKTRLEEPAEGFPMLKVKDIKSAYSHWQESKPILDKMVKQTRASSGVIWNSFKELEESELETVQREIPAPSFLIPLPKHYRASSTSLLDTDRSVFQWLDTQPPGSVLYVGFGSQSEVDPEDFLTIAQGLVDSGISFLWLVRPGFVKGSEWIEPLPDGFLGEKGRIVKSAPQQEVLAHGAIGAFWTHGGWNGTMESVCEGVPMIFSDFGLDQPLNARYMSDVLKVGVYLENGWDPAEIAAAIRRVMVDPEGEVIRENARVLKQKADRSIMKGGSSYESLESLVSYISSL

SEQ ID NO: 237 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALEELMEASESDEEVSCLITDALWYFAQSVADSLNLKRLVLQTGSLFNFHCLVSLPKFDELGYLDPDDKTRLEEPAEGFPMLKVKDIKSAYSHWQESKPILDKMVKQTRASSGVIWNSFKELEESELETVQREIPAPSFLIPLPKHYRASSTSLLDTDRSVFQWLDQQPPESVLYVGFGSQSEVDPKDFLEIADGLVASGQSFLWLVRPGFVKGSEWIEPLPEGYLGEKGRIVKSAPQQEVLAHGAIGAFWTHGGWNGTMESVCEGVPMIFSDFGLDQPLNARYMSDVLKVGVYLENGWNRDEIAAAIRYVMVDEAGEVIRENAKVLKQKADRSIMKGGSSYESLESLVSYISSL

SEQ ID NO: 238 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFRKLLEELMEASESDEEVSCLITDALWYFAQSVADSLNLKRLVLQTGSLFNFHCLVSLPKFDELGYLDPDDKTNLEEPAVGFPMLKVKDIKSAYSHWQESKPILMKMVKQTRASSGVIWNSFKELEESELETVQREIPAPSFLIPLPKHYRASSTSLLDTDRSVFQWLDTQPPSSVLYVGFGSQSEVDPADFLTIADGLVASGISFLWLVRPGFVKGSTWIEPLPDGYLGEKGRVVKSAPQQEVLAHGAIGAFWTHGGWNGTMESVCEGVPMIFSDFGLDQPLNARYMSEVLHVGVYLENGWDPAEIANAIRYVMRDPAGEPIRQNARVLKQKADRSIMKGGSSYESLESLVSYISSL

SEQ ID NO: 239 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFRKMLEELIAASESDETVSCLITDALWYFAQSVADSLNLRRLVLQTGSLFNFHCLVSLPKFDELGYLDPDDKTRLEEPAEGFPMLKVKDIKRAYSHWQESKPILEKMVKQTKASSGVIWNSFKELEESELETVWREIPAPSFLIPLPKHYRASSTSLLDTDRSVFQWLDQQPPGSVLYVGFGSQSEVDPKDFLEIADGLVASGQSFLWLVRPGFVKGSTWIEPLPDGYLGEKGRIVKSAPQQEVLAHGAIGAFWTHGGWNGTMESVCEGVPMIFSDFGLDQPLNARYMSEVLKVGVYLENGWEPAEIAAAIRRVMRDEAGEPIRENARVLKQKADRSIMKGGSSYESLESLVSYISSL

SEQ ID NO: 240 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRAELEKLIAASESDEEVSCLITDALWYFAQSVADSLNLKRLVLQTGSLFNFHCLVSLPKFDELGYLDPDDKTRLEEPAVGFPMLKVKDIKSAYSHWQESKPILDKMVKQTRASSGVIWNSFKELEESELETVQREIPAPSFLIPLPKHYRASSTSLLDTDRSVFQWLDQQPPSSVLYVGFGSQSEVDPEDFLKIAEGLVLSKQSFLWLVRPGFVKGSEWIEPLPDGFLGEKGRIVKSAPQQEVLAHGAIGAFWTHGGWNGTMESVCEGVPMIFSDFGLDQPLNARYMSDVLHVGVYLENGWDPEAIAKAIRTVMEDESGEKMRENARVLKQKADRSIMKGGSSYESLESLVSYISSL

SEQ ID NO: 241 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGAERFRELLEELILASESDEEVSCLITDALWYFAQSVADSLNLKRLVLQTGSLFNFHCLVSLPKFDELGYLDPDDKTRLEEPAEGFPMLKVKDIKSAYSHWQESKPILMKMVKQTRASSGVIWNSFKELEESELETVQREIPAPSFLIPLPKHYRASSTSLLDTDRSVFQWLDTQPPESVLYVGFGSQSEVDPEDFLKIAQGLVDSKQSFLWLVRPGFVKGSTWIEPLPEGFLGEKGRVVKSAPQQEVLAHGAIGAFWTHGGWNGTMESVCEGVPMIFSDFGLDQPLNARYMSEVLHVGVYLENGWNPEEIANAIRYVMTDPEGEPIRQNARVLKQKADRSIMKGGSSYESLESLVSYISSL

SEQ ID NO: 242 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFRKELEELILASESDEEVSCLITDALWYFAQSVADSLNLRRLVLQTGSLFNFHCLVSLPKFDELGYLDPDNKTRLEEPAEGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPETFLTIAEGLVKSGLSFLWLVRPGFVKGSTWIEPLPDGFLGEKGRIVKSAPQQEVLAHGAIGAFWTHGGWNGTMESVCEGVPMIFSDFGLDQPLNARYMSDVLKVGVYLENGWNPDEIAEAIRYVMVDEAGEPIRQNARVLKQKADRSIMKGGSSYESLESLVSYISSL

SEQ ID NO: 243 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALEELMAASESDEEVSCLITDALWYFAQDVADSLNLKRLVLQTGSLFNFHCLVSLPKFDELGYLDPDDKTRLEEPAEGFPELKVKDIKSAYSHWQESKPILDKMVKQTRASSGVIWNSFKELEESELETVQREIPAPSFLIPLPKHYRASSTSLLDTDRSVFQWLDTQPPESVLYVGFGSQSEVDEKDFLEIVRGLVLSGQSFLWLVRPGFVKGKEWIEPIPEGFLGEKGRIVKSAPQQEVLAHGAIGAFWTHGGWNGTMESVCEGVPMIFSDFGLDQPLNARYMSDVLHVGVYLENGWNPEEIAEAIRLVMVDERGEVIRQNARVLKQKADRSIMKGGSSYESLESLVSYISSL

SEQ ID NO: 244 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEEFRKELEKLMLASESDEEVSCLITDALWYFAQSVADSLNLKRLVLQTGSLFNFHCLVSLPKFDELGYLDPDDKTDLDKPAEGFPMLKVKDIKSAYSHWQESKPILEKMVKQTRASSGVIWNSFKELEESELETVQREIPAPSFLIPLPKHYRASSTSLLDTDRSVFQWLDQQPPGSVLYVGFGSQSEVDPKTFLEIARGLVDSGESFLWLVRPGFVKGKTWIEPLPEGFLGEKGRVVKSAPQQEVLAHGAIGAFWTHGGWNGTMEAVCEGVPMIFSDFGLDQPLNARYMSDVLKVGVYLENGWDPGEIAAAIRLVMRDEAGEPIRQNARVLKQKADRSIMKGGSSYESLESLVSYISSL

SEQ ID NO: 245 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALKELIEASESDEEVSCLITDALWYFAQSVADSLNLKRLVLQTGSLFNFHCLVSLPKFDELGYLDPDNKTDLEKPAPGFPMLKVKDIKSAYSHWQESKPILDKMVKQTRASSGVIWNSFKELEESELETVQREIPAPSFLIPLPKHYRASSTSLLDTDRSVFQWLDQQPPGSVLYVGFGSQSEVDPETFLKIAEGLVLSGQSFLWLVRPGFVKGSEWIEPLPDGFLGEKGRIVKSAPQQEVLAHGAIGAFWTHGGWNGTMESVCEGVPMIFSDFGLDQPLNARYMSDVLEVGVYLENGWDPEAIAAAIRRVMVDPEGEKIRENARVLKQKADRSIMKGGSSYESLESLVSYISSL

SEQ ID NO: 246 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLMNASESDEEVSCLITDALWYFAQDVADSLNLKRLVLQTGSLFNFHCLVSLPKFDELGYLDPDDKTNLEEPAEGFPWLKVKDIKSAYSHWQESKPILDKMVKQTRASSGVIWNSFKELEESELETVQREIPAPSFLIPLPKHYRASSTSLLDTDRSVFQWLDTQPPSSVLYVGFGSQSEVDEKDFLTIMEGLVKSGQSFLWLVRPGFVKGSEWIEPLPEGFLGEKGRIVKSAPQQEVLAHGAIGAFWTHGGWNGTMESVCEGVPMIFSDFGLDQPLNARYMSDVLKVGVYLENGWNADDIANAIRYVMVDEAGEVIRQNARVLKQKADRSIMKGGSSYESLESLVSYISSL

SEQ ID NO: 247 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKALEELMAASESDEEVSCLITDALWYFAQSVADSLNLKRLVLQTGSLFNFHCLVSLPKFDELGYLDPDNKERLDEPAPGFPMLKVKDIKSAYSHWQESKPILEKMVKQTRASSGVIWNSFKELEESELETVQREIPAPSFLIPLPKHYRASSTSLLDTDRSVFQWLDQQPPGSVLYVGFGSQSEVDPKTFLEIADGLVASGLSFLWLVRPGFVKGSTWIEPLPEGYLGEKGRVVKSAPQQEVLAHGAIGAFWTHGGWNGTMEAVCEGVPMIFSDFGLDQPLNARYMSDVLKVGVYLENGWDRDAIAAAIRRVMVDPEGEPIRQNAAVLKQKADRSIMKGGSSYESLESLVSYISSL

SEQ ID NO: 248 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADRFRKELELLMEASESDEEVSCLITDALWYFAQDVADSLNLKRLVLQTGSLFNFHCLVSLPKFDELGYLDPDDKTRLEEPVEGFPWLKVKDIKSAYSHWQESKPILMKMVKQTRASSGVIWNSFKELEESELETVQREIPAPSFLIPLPKHYRASSTSLLDTDRSVFQWLDTQPPESVLYVGFGSQSEVDEKDFLTIADGLVRSGQSFLWLVRPGFVKGSTWIEPLPEGYLGEKGRVVKSAPQQEVLAHGAIGAFWTHGGWNGTMESVCEGVPMIFSDFGLDQPLNARYMSDVLKVGVYLENGWDPDAIAAAIRRVMVDEAGEPIRQNARVLKQKADRSIMKGGSSYESLESLVSYISSL

SEQ ID NO: 249 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADQFRKLLEELMAASESDEEVSCLITDALWYFAQDVADSLNLKRLVLQTGSLFNFHCLVSLPKFDELGYLDPDDKTNLDKPAEGFPMLKVKDIKSAYSHWQESKPILEKMVKQTRASSGVIWNSFKELEESELETVQREIPAPSFLIPLPKHYRASSTSLLDTDRSVFQWLDTQPPRSVLYVGFGSQSEVDEKDFLKIVEGLVASKQSFLWLVRPGFVKGSTWIEPIPEGFLGEKGRVVKSAPQQEVLAHGAIGAFWTHGGWNGTMEAVCEGVPMIFSDFGLDQPLNARYMSDVLKVGVYLENGWDPGEIANAIRRVMVDPEGEPIRENAEVLKQKADRSIMKGGSSYESLESLVSYISSL

SEQ ID NO: 250 - protein, 457 aa, synthetic construct
MPNTNTTVRRRRRIILFPVPFPGHLNPMLQLANVLYSKGFSITIFHTNFNKPKTSNYPHFTFVTILDNDPQDEWLRNLPTHGPGVGARIPVINKHGADQFRKLLELLMSASESDEEVSCLITDALWYFAQSVADSLNLKRLVLQTGSLFNFHCLVSLPKFDELGYLDPDDKTRLDEPAVGFPELKVKDIKSAYSHWQESKPILEKMVKQTRASSGVIWNSFKELEESELETVQREIPAPSFLIPLPKHYRASSTSLLDTDRSVFQWLDTQPPESVLYVGFGSQSEVDPKTFLEIAEGLVESKQPFLWLVRPGFVKGYTWIEPLPEGFLGEKGRVVKSAPQQEVLAHPAIGAFWTHGGWNGTMEAVCEGVPMIFSDFGLDQPLNARYMSDVLKVGVYLDNGWERGKIAEAIRYVMVDEKGEYIRQNARVLKQKADRSIMKGGSSYESLESLVSYISSL

SEQ ID NO: 251 - protein, 457 aa, synthetic construct
MPNTNTTVRRRRRIILFPVPFPGHWNPMLQLANVLYSKGFSITILHTNFNKPKTSNYPHFTFIFTLDNDPQDEWLRNLPTHGPGVGARIPVINKHGADEFRKTLEDLIAASESDEEVSCLITDALWYFAQSVADSLNLKRLVLQTGSLFNFHCLVSLPKFDELGYLDPDDKTRLEEPVEGFPYLKVKDIKSAYSHWQESKPILMKMVKQTRASSGVIWNSFKELEESELETVQREIPAPSFLIPLPKHYRASSTSLLDTDRSVFAWLDTQPPESVLYVGFGSQSEVDPKTFLEIAEGLVASKQPFLWLVRPGFVKGKEWIEPLPEGFLGEKGRVVKSAPQQEVLAHPAIGAFWTHGGWNGTMESVCEGVPMIFSDFGLDQPLNARYMSEVLKVGVYLDNGWERDAIAAAIRYVMVDEAGEPIRENARVLKQKADRSIMKGGSSYESLESLVSYISSL

SEQ ID NO: 252 - protein, 457 aa, synthetic construct
MPNTNTTVRRRRRIILFPVPFPGHLNPMLQLANVLYSKGFSITIFHTNFNKPKTSNYPHFTFRTILDNDPQDEWLRNLPTHGPGVGARIPVINKHGADQFRKLLEELMLASESDETVSCLITDALWYFAQDVADSLNLRRLVLQTGSLFNFHCLVSLPKFEELGYLDPNDKTRLEEPAEGFPMLKVKDIKRAYSHWQESKPILEKMVKQTKASSGVIWNSFKELEESELETVWREIPAPSFLIPLPKHYRASSTSLLDTDRSVFQWLDTQPPSSVLYVGFGSQSEVTPEDFLKIVEGLVASGLPFLWLVRPGFVKGSEWIEPLPEGFLGEKGRIVKSAPQQEVLAHPAIGAFWTHGGWNGTMESVCEGVPMIFSDFGLDQPLNARYMSEVKKVGVYLDNGWEPGEIAAAIRYVMVDPEGEYIRQNARVLKQKADRSIMKGGSSYESLESLVSYISSL

SEQ ID NO: 253 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELELLINASESDEEVSCLITDALWYFAQDVADSLNLKRLVLQTGSLFNFHCLVSLPKFDELGYLDPNNKERLDEPVEGFPMLKVKDIKSAYSHWQESKPILEKMVKQTRASSGVIWNSFKELEESELETVQREIPAPSFLIPLPKHYRASSTSLLDTDRSVFAWLDQQPPGSVLYVGFGSQSEVDPETFLEIVRGLVLSKQSFLWLVRPGFVKGSAWIEPLPPGFLGEKGRVVKSAPQQEVLAHPAIGAFWTHGGWNGTMEAVCEGVPMIFSDFGLDQPLNARYMSDVLKVGVYLDNGWDPEEIAAAIRRVMVDESGAPIRENARVLKQKADRSIMKGGSSYESLESLVSYISSL

SEQ ID NO: 254 - protein, 457 aa, synthetic construct
MPNTNTTVRRRRRIILFPVPFPGHLNPMLQLANVLYSKGFSITIFHTEFNKPKTSNYPHFTFRTILDNDPQDEWLRNLPTHGPGVGARIPVINKHGADRFRKELEELMAASESDEEVSCLITDALWYFAQDVADSLNLKRLVLQTGSLFNFHCLVSLPKFEELGYLDPTDKTGLDEPVEGFPWLKVKDIKSAYSHWQESKPILEKMVKQTRASSGVIWNSFKELEESELETVQREIPAPSFLIPLPKHYRASSTSLLDTDRSVFAWLDTQPPESVLYVGFGSQSEVTPEDFLTIAQGLVDSKQPFLWLVRPGFVKGYAWIEPLPEGFLGEKGRVVKSAPQQEVLAHPAIGAFWTHGGWNGTMEAVCEGVPMIFSDFGLDQPLNARYMSDVLKVGVYLDNGWNPEEIANAIRRVMVDEEGEPIRQNARVLKQKADRSIMKGGSSYESLESLVSYISSL

SEQ ID NO: 255 - protein, 457 aa, synthetic construct
MPNTNTTVRRRRRIILFPVPFPGHLNPMLQLANVLYSKGFSITIFHTEFNKPKTSNYPHFTFRTILDNDPQDEWLRNLPTHGPGVGARIPVINKHGADQFRKLLEELIAASESDEEVSCLITDALWYFAQDVADSLNLKRLVLQTGSLFNFHCLVSLPKFEELGYLDPSDKTRLDEPAVGFPELKVKDIKSAYSHWQESKPILEKMVKQTRASSGVIWNSFKELEESELETVQREIPAPSFLIPLPKHYRASSTSLLDTDRSVFDWLDQQPPSSVLYVGFGSQSEVTPKEFLEIADGLVASGQPFLWLVRPGFVKGYDWIEPLPDGYLGEKGRVVKSAPQQEVLAHPAIGAFWTHGGWNGTMEAVCEGVPMIFSDFGLDQPLNARYMSDVKKVGVYLDNGWNPEEIAAAIRLVMVDPEGEPIRQNARVLKQKADRSIMKGGSSYESLESLVSYISSL

SEQ ID NO: 256 - protein, 457 aa, synthetic construct
MPNTNPTTVRRKTVVMFPVPFPGHLNPMLQLANVLYRRGFRIVILYTNFNAPKTSLYPHFEFRQILDNDPQPEWLRNLPTTGPGVGARIPVINKHGAEPFKATLQECIADTPSDEEVACVIADALWYFAQPVAKELNLPLLVLQTGSLFNFHCLVCLPEFIRLGLLDPNTSWADDAPVPGFPILKVKDIRRAYSHIQESKPILMKMVETTREASGVIWNSAKELEESELETIQREIPAPSFLLPLPKHYRASSTSLLDTDPSTAAWLDTQPERSVLYVGFGSQSSLEPEDFLNLAEGLVRSGQPFLWLVRPGFVKGYEWTELLPDGFLGERGRIVKSAPQQEVLAHPAIGAFWTHGGWNGTMEAVCEGVPMIFSDFGLDQPLNAAYMTKVKKIGVYLDRGFRVDDIVRAVREVMVDPKGKEYRANAEELKEKLDRSIAPGGSSYESLERLQSYISSL

SEQ ID NO: 257 - protein, 457 aa, synthetic construct
MPNTNPTTVRRKRVVMFPVPFPGHLNPMLQLANVLYRRGFRITILYTNFNAPKTSLYPHFEFRQILDNDPQPEWLRNLPTTGPGVGARIPVINKHGAEPLKATLRECIAATPSDEEVACVIADALWYFAAPVAKELNLPLLVLQTGSLFNFHCLVCLPQFIELGLLDPNSSWADDAPVPGFPELRVRDIRRAYSHIQESKPILMKMVETTREASGVIWNSAKELEESELETIQREIPAPSFLLPLPKHYRASSTSLLDTDPSTAAWLAGQPPESVVYVGFGSQSSLEPEDALNLARGLVESGLPFLWLVRPGFIKGHEWTEILPDGFLGERGRIVKSAPQQEVLAHPAIGAFWTHGGWNGTMEAVCEGVPMIFSDFGLDQPLNAVYMSKVKKIGVYLDNGFRVDEIVRALREVMVEPKGKEYRANARELKEKLDRSIAPGGSSYESLERLQSYISSL

SEQ ID NO: 258 - protein, 457 aa, synthetic construct
MPNTNPTTVRRKWVVMFPVPFPGHLNPMLQLANVLYRRGFKIIILYTNFNAPKTSLYPHFEFRQILDNDPQPEWLRNLPTTGPGVGARIPVINKHGAEPFKATLEECIADTPSDEEVACVIADALWYFAAPVAKELNLPLLVLQTGSLFNFHCLVCLPEFIELGLLDPNSSWADDAPVPGFPELKVRDIRRAYSHIQESKPILMKMVEQTRASSGVIWNSAKELEESELETIQREIPAPSFLLPLPKHYRASSTSLLDTDPSTAAWLAGQPERSVLYVGFGSQSSLEPEDALNLAEGLVRSGVPFLWLVRPGFIKGYEWTEILPDGFLGERGRIVKSAPQQEVLAHPAIGAFWTHGGWNGTMEAVCEGVPMIFSDFGLDQPLNARYMTKVKKIGVYLDDGFKVERIVEAIRRVMVEPEGEEYRKNALELKEKLDRSIAPGGSSYESLERLQSYISSL

SEQ ID NO: 259 - protein, 457 aa, synthetic construct
MPNTNPTTVRRKTVVMFPVPFPGHLNPMLQLANVLYRRGFRITILYTNFNAPKTSLYPHFTFRNILDNDPQPEWLRNLPTTGPGVGARIPVINKHGAEPFKKVLEECIRETPSDEEVACVIADALWYFAYPVTKELNLPLLVLQTGSLFNFHCLVCLPEFIELGLLDPNSSWPDDAPVPGFPILRVRDIRRAYSHIQESKPILMKMVETTRESSGVIWNSAKELEESELETIQREIPAPSFLLPLPKHYRASSTSLLDTDPSTAAWLDGQPPESVVYVGFGSQSSIEPEDFLNLAEGLVRSGLPFLWLVRPGFVKGYEWTELLPDGFLGERGRIVKSAPQQEVLAHPAIGAFWTHGGWNGTMEAVCEGVPMIFSDFGLDQPLNARYMAEVKKIGVYLDRGFRVEEIVRALREVMVEPKGREYRKNARELKEKLDRSIAPGGSSYESLERLQSYISSL

SEQ ID NO: 260 - protein, 457 aa, synthetic construct
MPNTNPTTVRRRTVVMFPVPFPGHLNPMLQLANVLYRRGFRIVILYTNFNAPKTSLYPHFQFVQILDNDPQPEWLRNLPTTGPGVGARIPVINKHGAEPLKATLEQCIADTPSDEEVACVITDALWYFAGPVAEELGLPRLVLQTGSLFNFHCLVCLPEFIRLGLLDPNSSWADDAPVPGFPELKVRDIRRAYSHIQESKPILMKMVEETRRSSGVIWNSAKELEESELETIQREIPAPSFLLPLPKHYRASSTSLLDTDPSTAAWLDGQPERSVLYVGFGSQSSIEPEEALNIAEGLVASGVPFLWLVRPGFVKGYEWTELLPDGFLGERGRIVKSAPQQEVLAHPAIGAFWTHGGWNGTMEAVCEGVPMIFSDFGLDQPLNAVYMTKVKKIGVYLDRGFVKEEIVRAIREVMVDPKGEEYRKNALELKEKLDRSIAPGGSSYESLERLQSYISSL

SEQ ID NO: 261 - protein, 457 aa, synthetic construct
MPNTNPTTVRRRRVVMFPVPFPGHLNPMLQLANVLYRRGFDIIILYTNFNAPKTSLYPHFQFRQILDNDPQPEWLRNLPTTGPGVGARIPVINKHGAAPFKATLQQCIADTPSDEEVACVITDALWYFAQPVTEELNLKRLVLQTGSLFNFHCLVCLPEFLELGLLDPNSSWADDAPVPGFPILKVKDIRRAYSHIQESKPILMKMVEETRKSSGVIWNSAKELEESELETIQREIPAPSFLLPLPKHYRASSTSLLDTDPSTAAWLDGQPPESVLYVGFGSQSSLEPADALALAEGLVASGVPFLWLVRPGFVKGHEWTELLPDGFLGERGRIVKSAPQQEVLAHPAIGAFWTHGGWNGTMEAVCEGVPMIFSDFGLDQPLNARYMSEVKKIGVYLDDGFRVDRIVAAVREVMVDPRGREMRANARELKDKLDRSIAPGGSSYESLERLQSYISSL

SEQ ID NO: 262 - protein, 457 aa, synthetic construct
MPNTNPTTVRRRRVVMFPVPFPGHLNPMLQLANVLYRRGFEITILYTNFNAPKTSLYPHFQFRQILDNDPQPEWLRNLPTTGPGVGARIPVINKHGAAPFKATLQQCIAETPSDEEVACVITDALWYFAEPVARELNLKRLVLQTGSLFNFHCLVCLPQFLELGLLDPNSSWADDAPVPGFPILRVKDIRRAYSHIQESKPILMKMVEETRKSSGVIWNSAKELEESELETIQREIPAPSFLLPLPKHYRASSTSLLDTDPSTAAWLDGQPPESVLYVGFGSQSSLDPEEFLNLAEGLVRSGVPFLWLVRPGFVKGYEWIELLPDGFLGERGRIVKSAPQQEVLAHPAIGAFWTHGGWNGTMEAVCEGVPMIFSDFGLDQPLNARYMTEKLKIGVYLDDGFRVEEIVRAIREVMVEPKGKEYRENARKLKDKLDRSIAPGGSSYESLERLQSYISSL

SEQ ID NO: 263 - protein, 457 aa, synthetic construct
MPNTNPTTVRRRRVVMFPVPFPGHLNPMLQLANVLYRRGFSITILYTNFNAPKTSLYPHFQFRQILDNDPQPEWLRNLPTTGPGVGARIPVINKHGAEPFRQTLEQCIQETPSDEEVACVITDALWYFAQPVTESLNLKRLVLQTGSLFNFHCLVCLPQFLELGLLDPNSSHADDAPVPGFPILKVKDIRRAYSHIQESKPILMKMVEETRKSSGVIWNSAKELEESELETIQREIPAPSFLLPLPKHYRASSTSLLDTDPSTAAWLDGQPERSVLYVGFGSQSSLEPEDFLNLAEGLVESGVPFLWLVRPGFVKGHEWTELLPDGFLGEKGRIVKSAPQQEVLAHPAIGAFWTHGGWNGTMEAVCEGVPMIFSDFGLDQPLNARYMTEKLKIGVYLDEGFQKERIVEAIRKVMVEPEGEEYRKNALELKDKLDRSIAPGGSSYESLERLQSYISSL

SEQ ID NO: 264 - protein, 457 aa, synthetic construct
MPNTNPTTVRRRRVVMFPVPFPGHLNPMLQLANVLYRRGFEITILHTNFNAPKTSLYPHFQFRQILDNDPQPEWLRNLPTTGPGVGARIPVINKHGAAPFKATLQQCIAETPSDEEVACVITDALWYFAQPVTRELGLKRLVLQTGSLFNFHCLVCLPKFLADGYLDPNTKWADDAPVEGFPFLKVKDIRRAYSHIQESKPILMKMVEETRASSGVIWNSAKELEESELETIQREIPAPSFLLPLPKHYRASSTSLLDTDPSTAQWLDQQPENSVLYVGFGSQSSLDPEDFLNIAEGLVESGISFLWLVRPGFVKGHEWIELLPDGFLGERGRIVKSAPQQEVLAHKAIGAFWTHGGWNGTMEAVCEGVPMIFSDFGLDQPLNARYMSEVLKVGVYLENGFRKEDIVRAIKEVMVDEKGKEYRANAQKLKDKLDRSIAPGGSSYESLERLQSYISSL

SEQ ID NO: 265 - protein, 457 aa, synthetic construct
MPNTNPTTVRRRTVVMFPVPFPGHLNPMLQLANVLYRRGFRITILYTNFNAPKTSLYPHFQFRNILDNDPQPEWLRNLPTTGPGVGARIPVINKHGAEPFRATLEQCIADTPSDEEVACVITDALWYFAEPVTRSLNLKRLVLQTGSLFNFHCLVCLPEFLELGLLDPNTSWPDDAPVPGFPILKVKDIRRAYSHIQESKPILMKMVEETRKSSGVIWNSAKELEESELETIQREIPAPSFLLPLPKHYRASSTSLLDTDPSTAAWLDQQPPESVLYVGFGSQSSLEPEDALALAEGLVRSGQPFLWLVRPGFVKGYEWTELLPDGFLGEKGRIVKSAPQQEVLAHPAIGAFWTHGGWNGTMEAVCEGVPMIFSDFGLDQPLNARYMTEVKKIGVYLDEGFRVEAIVRAVREVMVDPKGEVYRKNARELKDKLDRSIAPGGSSYESLERLQSYISSL

SEQ ID NO: 266 - protein, 457 aa, synthetic construct
MPNTNPTTVRRRRVVMFPVPFPGHLNPMLQLANVLYRRGFEITILYTNFNAPKTSLYPHFQFRQILDNDPQPEWLRNLPTTGPGVGARIPVINKHGAAPFKAVLEECIADTPSDEEVACVITDALWYFAGPVTRELNLKRLVLQTGSLFNFHCLVCLPEFLELGLLDPNTKHADDAPVPGFPFLKVKDIRRAYSHIQESKPILMKMVEETRRSSGVIWNSAKELEESELETIQREIPAPSFLLPLPKHYRASSTSLLDTDPSTAAWLDQQPPESVLYVGFGSQSSLDPEDFLNLAEGLVRSGLPFLWLVRPGFVKGHEWIELLPDGFLGEKGRIVKSAPQQEVLAHPAIGAFWTHGGWNGTMEAVCEGVPMIFSDFGLDQPLNARYMSEVKKIGVYLDEGFRVERIVEAVRRVMVEPEGRVYRENARKLKDKLDRSIAPGGSSYESLERLQSYISSL

SEQ ID NO: 267 - protein, 457 aa, synthetic construct
MPNTNPTTVRRRRVVMFPVPFPGHLNPMLQLANVLYRRGFAITILYTNFNAPKTSLYPHFQFRTILDNDPQPEWLRNLPTTGPGVGARIPVINKHGAEEFRAALEQCIADTPSDEEVACVITDALWYFAGPVTESLGLKRLVLQTGSLFNFHCLVCLPQFLELGLLDPDTPWADDAPVPGFPFLRVRDIRRAYSHIQESKPILMKMVEETRRSSGVIWNSAKELEESELETIQREIPAPSFLLPLPKHYRASSTSLLDTDPSTAAWLDQQPPRSVLYVGFGSQSSLEPEDFLNLAEGLRRSGHRFLWLVRPGFVKGYEWIELLPDGFLGERGRIVKSAPQQEVLAHPAIGAFWTHGGWNGTMEAVCEGVPMIFSDFGLDQPLNARYMTEVKNIGIYLDEGFRVEEIVRAVNTVMREPKGREMAANAQALKDKLDRSIAPGGSSYESLERLQSYISSL

SEQ ID NO: 268 - protein, 457 aa, synthetic construct
MPNTNPTTVRRRRVVMFPVPFPGHLNPMLQLANVLYRRGFSITILYTNFNAPKTSLYPHFQFRQILDNDPQPEWLRNLPTTGPGVGARIPVINKHGAAPFRQTLEQCIADTPSDEEVACVITDALWYFAQPVAQELNLPLLVLQTGSLFNFHCLVCLPQFIELGLLDPNTKWADDAPVPGFPILRVRDIRRAYSHIQESKPILMKMVEQTRASSGVIWNSAKELEESELETIQREIPAPSFLLPLPKHYRASSTSLLDTDPSTAAWLAQQPERSVLYVGFGSQSSLEPEEALEIARGLVKSKQPFLWLVRPGFIKGYEWTELLPDGFLGERGRIVKSAPQQEVLAHPAIGAFWTHGGWNGTMEAVCEGVPMIFSDFGLDQPLNARYMTEKLKIGVYLDNGFIEEEIVRAIREVMVDPKGEEYRANALALKEKLDRSIAPGGSSYESLERLQSYISSL

SEQ ID NO: 269 - protein, 457 aa, synthetic construct
MPNTNPTTVRRRTVVMFPVPFPGHLNPMLQLANVLYRRGFRITILYTNFNAPKTSLYPHFQFVQILDNDPQPEWLRNLPTTGPGVGARIPVINKHGAEEFRKTLEICMRDTPSDEEVACVITDALWYFAQPVADSLGLKRLVLQTGSLFNFHCLVCLPKFLELGLLDPDTKWADDAPVPGFPFLKVRDIRRAYSHIQESKPILMKMVEETRRSSGVIWNSAKELEESELETIQREIPAPSFLLPLPKHYRASSTSLLDTDPSTAAWLDQQPEESVLYVGFGSQSSLEPEDFLNLAKGLVLSKQPFLWLVRPGFVKGYEWIELLPDGFLGERGRIVKSAPQQEVLAHPAIGAFWTHGGWNGTMEAVCEGVPMIFSDFGLDQPLNARYMTEVLNIGIYLEDGFIVGNIVKAVNEIMVEPKGKEYRANAKALKDKLDRSIAPGGSSYESLERLQSYISSL

SEQ ID NO: 270 - protein, 457 aa, synthetic construct
MPNTNPTTVRRRRVVMFPVPFPGHLNPMLQLANVLYRRGFSITILYTNFNAPKTSLYPHFQFVQILDNDPQPEWLRNLPTTGPGVGARIPVINKHGAEPFKKTLEQCIADTPSDEEVACVITDALWYFAQPVTESLGLKRLVLQTGSLFNFHCLVCLPQFLELGLLDPNSPWADDAPVPGFPMLRVKDIRRAYSHIQESKPILMKMVEETRKSSGVIWNSAKELEESELETIQREIPAPSFLLPLPKHYRASSTSLLDTDPSTAAWLDQQPEESVLYVGFGSQSSLEPKDALEIARGLVLSKQPFLWLVRPGFIKGYEWTELLPDGFLGENGRIVKSAPQQEVLAHKAIGAFWTHGGWNGTMEAVCEGVPMIFSDFGLDQPLNARYMTEVKKIGIYLDNGFKVDDIIKAVNEVMVDEKGKVYRENAKKLKDKLDRSIAPGGSSYESLERLQSYISSL

SEQ ID NO: 271 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRRLLEELISASESDEEVSCLITDALWYFAQSVADSLNLKRLVLQTGSLFNFHCLVSLPKFDELGYLDPDDKSRLDEPAVGFPMLKVKDIKSAYSHWQESKPILEKMVKQTRASSGVIWNSFKELEESELETVQREIPAPSFLIPLPKHYRASSTSLLDTDRSVFQWLDQQPPSSVLYVGFGSQSEVDPKDFLEIARGLVLSGQSFLWLVRPGFVKGSTWIEPLPEGFLGEKGRVVKSAPQQEVLAHGAIGAFWTHGGWNGTMEAVCEGVPMIFSDFGLDQPLNARYMSDVLHVGVYLENGWDPQRIAEAIRLVMVDERGAPMRQNARVLKQKADRSIMKGGSSYESLESLVSYISSL

SEQ ID NO: 272 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRRLLQELILASESDEEVSCLITDALWYFAQSVADSLNLRRLVLQTGSLFNFHCLVSLPKFDELGYLDPDDKSRLDEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLEIARGLVLSGQSFLWLVRPGFVKGSTWIEPLPEGFLGEKGRIVKSAPQQEVLAHGAIGAFWTHGGWNGTMESVCEGVPMIFSDFGLDQPLNARYMSEVLHVGVYLENGWNPEEIARAINLVMRDPEGEYMRQNARVLKQKADRSIMKGGSSYESLESLVSYISSL

SEQ ID NO: 273 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKLLEEEILASESDEEVSCLITDALWYFAQSVADSLNLRRLVLQTGSLFNFHCLVSLPKFDELGYLDPDDKTRLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLEIARGLVLSKQSFLWLVRPGFVKGSTWIEPLPEGFLGEKGRIVKSAPQQEVLAHGAIGAFWTHGGWNGTMESVCEGVPMIFSDFGLDQPLNARYMSEVLHVGVYLENGWEPERIARAIRLVMVDEEGKPMRENARVLKQKADRSIMKGGSSYESLESLVSYISSL

SEQ ID NO: 274 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKELEELISASESDEEVSCLITDALWYFAQSVADSLNLRRLVLQTGSLFNFHCLVSLPKFDELGYLDPDDKTRLDEPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPADFLAIARGLVLSGQSFLWLVRPGFVKGSTWIEPLPPGFLGEKGRIVKSAPQQEVLAHGAIGAFWTHGGWNGTMESVCEGVPMIFSDFGLDQPLNARYMSEVLHVGVYLENGWNPEEIANAIRLVMVDERGAPMRQNARVLKQKADRSIMKGGSSYESLESLVSYISSL

SEQ ID NO: 275 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGADEFRKLLEEEISASESDEEVSCLITDALWYFAQSVADSLNLKRLVLQTGSLFNFHCLVSLPKFDELGYLDPDDKTRLDEPAVGFPMLKVKDIKSAYSHWQESKPILMKMVKQTRASSGVIWNSFKELEESELETVQREIPAPSFLIPLPKHYRASSTSLLDTDRSVFQWLDQQPPSSVLYVGFGSQSEVDPKDFLKIAQGLVDSGQSFLWLVRPGFVKGSTWIEPLPEGFLGEKGRVVKSAPQQEVLAHGAIGAFWTHGGWNGTMESVCEGVPMIFSDFGLDQPLNARYMSEVLHVGVYLENGWDPKEIANAIRKVMVDEEGKPMRENARVLKQKADRSIMKGGSSYESLESLVSYISSL

SEQ ID NO: 276 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKELEELMSASESDEEVSCLITDALWYFAQSVADSLNLRRLVLQTGSLFNFHCLVSLPKFDELGYLDPDDKTDLDKPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPKDFLKIAEGLVKSGQSFLWLVRPGFVKGSTWIEPLPDGFLGEKGRIVKSAPQQEVLAHGAIGAFWTHGGWNGTMESVCEGVPMIFSDFGLDQPLNARYMSEVLHVGVYLENGWEPEEIARAIRLVMVDEEGAYMRENARVLKQKADRSIMKGGSSYESLESLVSYISSL

SEQ ID NO: 277 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKLLEEEISASESDEEVSCLITDALWYFAQSVADSLNLRRLVLQTGSLFNFHCLVSLPKFDELGYLDPDDKTNLEEPAVGFPMLKVKDIKRAYSHWQESKPILEKMVKQTKASSGVIWNSFKELEESELETVWREIPAPSFLIPLPKHYRASSTSLLDTDRSVFQWLDQQPPRSVLYVGFGSQSEVDPEDFLRIAEGLVKSGQSFLWLVRPGFVKGSTWIEPLPPGFLGEKGRIVKSAPQQEVLAHGAIGAFWTHGGWNGTMESVCEGVPMIFSDFGLDQPLNARYMSEVLKVGVYLENGWDPEAIARAIRLVMVDEEGRPIRENARVLKQKADRSIMKGGSSYESLESLVSYISSL

SEQ ID NO: 278 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFKALLEEEIAASESDEEVSCLITDALWYFAQSVADSLNLRRLVLQTGSLFNFHCLVSLPKFDELGYLDPDDKSNLDKPAVGFPMLKVKDIKRAYSHWQESKPILEKMVKQTKASSGVIWNSFKELEESELETVWREIPAPSFLIPLPKHYRASSTSLLDTDRSVFQWLDQQPPSSVLYVGFGSQSEVDPEDFLKIAEGLKLSGQSFLWLVRPGFVKGSTWIEPLPEGFLGEKGRIVKSAPQQEVLAHGAIGAFWTHGGWNGTMESVCEGVPMIFSDFGLDQPLNARYMSEVLHVGVYLENGWDPERIAEAIRLVMVDPAGAPMRENARVLKQKADRSIMKGGSSYESLESLVSYISSL

SEQ ID NO: 279 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAERFKKELEELISASESDEEVSCLITDALWYFAQSVADSLNLKRLVLQTGSLFNFHCLVSLPKFDELGYLDPDDKTRLDEPAEGFPMLKVKDIKSAYSHWQESKPILEKMVKQTRASSGVIWNSFKELEESELETVQREIPAPSFLIPLPKHYRASSTSLLDTDRSVFQWLDQQPPRSVLYVGFGSQSEVDPEDFLKIAEGLVKSGLSFLWLVRPGFVKGSTWIEPLPEGFLGEKGRVVKSAPQQEVLAHGAIGAFWTHGGWNGTMEAVCEGVPMIFSDFGLDQPLNARYMSDVLKVGVYLENGWEPERIANAIRLVMVDERGAPIRENARVLKQKADRSIMKGGSSYESLESLVSYISSL

SEQ ID NO: 280 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFKKLLEEEIAASESDEEVSCLITDALWYFAQSVADSLNLRRLVLQTGSLFNFHCLVSLPKFDELGYLDPDDKTRLDEPAEGFPMLKVKDIKRAYSHWQESKPILEKMVKQTKASSGVIWNSFKELEESELETVWREIPAPSFLIPLPKHYRASSTSLLDTDRSVFQWLDQQPPESVLYVGFGSQSEVDPEDFLKIAEGLVRSGLSFLWLVRPGFVKGSTWIEPLPEGFLGEKGRIVKSAPQQEVLAHGAIGAFWTHGGWNGTMESVCEGVPMIFSDFGLDQPLNARYMSEVLKVGVYLENGWEPERIAEAIRLVMVDERGRPIRQNARVLKQKADRSIMKGGSSYESLESLVSYISSL

SEQ ID NO: 281 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAERFRKLLEEEIAASESDEEVSCLITDALWYFAQSVARSLNLKRLVLQTGSLFNFHCLVSLPKFDELGYLDPDDKTNLDKPAEGFPMLKVKDIKSAYSHWQESKPILEKMVKQTRASSGVIWNSFKELEESELETVQREIPAPSFLIPLPKHYRASSTSLLDTDRSVFQWLDQQPPRSVLYVGFGSQSEVDPADFLRIAEGLVLSGQSFLWLVRPGFVKGSTWIEPLPEGFLGEKGRVVKSAPQQEVLAHGAIGAFWTHGGWNGTMEAVCEGVPMIFSDFGLDQPLNARYMSDVLKVGVYLENGWDPAAIAEAIRLVMVDPAGEPIRRNARVLKQKADRSIMKGGSSYESLESLVSYISSL

SEQ ID NO: 282 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGAERFKKLLEEEISASESDEEVSCLITDALWYFAQSVADSLNLKRLVLQTGSLFNFHCLVSLPKFDELGYLDPDDKTNLDEPAEGFPMLKVKDIKSAYSHWQESKPILMKMVKQTRASSGVIWNSFKELEESELETVQREIPAPSFLIPLPKHYRASSTSLLDTDRSVFQWLDQQPPESVLYVGFGSQSEVDPEDFLRIAEGLVLSGQSFLWLVRPGFVKGSTWIEPLPEGFLGEKGRVVKSAPQQEVLAHGAIGAFWTHGGWNGTMESVCEGVPMIFSDFGLDQPLNARYMSEVLHVGVYLENGWDPEAIARAIRLVMVDPEGAPIRENARVLKQKADRSIMKGGSSYESLESLVSYISSL

SEQ ID NO: 283 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAETFKKLLQEEMAASESDETISCLITDALWYFAQEVADELNLKRLVLQTGSLFNFHCLVSLPKFDELGYLDPDNKENLDEPAEGFPELKVKDIKSAYSHWQESKPILEKMVKQTRASSGVIWNSFKELEESELETVQREIPAPSFLIPLPKHYRASSTSLLDTDRSVFQWLDGQPPESVLYVGFGSQSEVDEEDFLKIAEGLKRSGLSFLWLVRPGFVKGHTWIEPLPEGFLGPQGRVVKSAPQQEVLAHGAIGAFWTHGGWNGTMEAVCEGVPMIFSDFGLDQPLNARYMSNVLKVGVYLEDGWEPERIAEAIRLVMVDERGRPIRENAKVLKQKADRSIMKGGSSYESLESLVSYISSL

SEQ ID NO: 284 - protein, 457 aa, synthetic construct
MPNTNTTVRRRRRIALFPVPFPGHWNPMLQLANVLYSKGFSITIFHTNFNKPKTSNYPHFTFVTILDNDPQDEWLRNLPTHGPGVGARIPVINKHGKERFKKELELLMSASESDEELSCLITDALWYFAQDVADELNLKRLVLQTGSLFNFHCLVSLPKFDELGYLDPNNKTDLEKPAEGFPWLKVKDIKSAYSHWQESKPILMKMVKQTRASSGVIWNSFKELEESELETVQREIPAPSFLIPLPKHYRASSTSLLDTDRSVFDWLDQQPPRSVLYVGFGSQSEVEPEDFLAIARGLVDSGLPFLWLVRPGFVKGHTWIEPLPEGFLGPQGRVVKSAPQQEVLAHPAIGAFWTHGGWNGTMESVCEGVPMIFSDFGLDQPLNARYMSEVRKVGVYLDDGWEPERIAAAIRRVMVDEEGAPIRQNAEVLKQKADRSIMKGGSSYESLESLVSYISSL

SEQ ID NO: 285 - protein, 457 aa, synthetic construct
MPNTNTTVRRRRRIILFPVPFPGHWNPMLQLANVLYSKGFSITILHTNFNKPKTSNYPHFTFVTILDNDPQDEWLRNLPTHGPGVGARIPVINKHGAERLRKILEELMAASESDEEISCLITDALWYFAEDVARSLNLKRLVLQTGSLFNFHCLVSLPQFDELGYLDPNDKTRLDEPAEGFPELLVKDIKSAYSHWQESKPILMKMVKQTRASSGVIWNSFKELEESELETVQREIPAPSFLIPLPKHYRASSTSLLDTDRSVFDWLDQQPPRSVLYVGFGSQSEVEPEDFLAIARGLVLSGVPFLWLVRPGFVKGHTWIEPLPEGFLGPLGRVVKSAPQQEVLAHPAIGAFWTHGGWNGTMESVCEGVPMIFSDFGLDQPLNARYMSEVKKIGVYLDNGWEPEAIAAAIRLVMVDPAGEPIRQNARVLKQKADRSIMKGGSSYESLESLVSYISSL

SEQ ID NO: 286 - protein, 457 aa, synthetic construct
MPNTNTTVRRRRRIVLFPVPFPGHLNPMLQLANVLYSKGFSITILHTSFNKPKTSNYPHFTFVTILDNDPQDEWLRNLPTHGPGVGARIPVINKHGADRLKKILEELISASESDEEVSCLITDALWYFAQSVADSLNLKRLVLQTGSLFNFHCLVSLPKFEELGYLDPDNKTRLDEPAEGFPELKVKDIKSAYSHWQESKPILEKMVKQTRESSGVIWNSFKELEESELETVQREIPAPSFLIPLPKHYRASSTSLLDTDRSVFDWLAQQPPESVLYVGFGSQSEVEPEDFLAIARGLVASGLPFLWLVRPGFVKGHTWIEPLPPGFLGPLGRVVKSAPQQEVLAHPAIGAFWTHGGWNGTMEAVCEGVPMIFSDFGLDQPLNARYMSDVLKVGVYLDNGWEPEAIARAIRLVMVDEAGRPIRENARVLKQKADRSIMKGGSSYESLESLVSYISSL

SEQ ID NO: 287 - protein, 457 aa, synthetic construct
MPNTNTTVRRTRRIILFPVPFPGHLNPMLQLANVLYSKGFSITIFHTNFNKPKTSNYPHFTFRFILDNDPQDEWLRNLPTHGPGVGARIPVINKHGAEEFRKALEEEISASESDEEVSCLITDALWYFAQSVADSLNLKRLVLQTGSLFNFHCLVSLPKFDELGYLDPDDKTRLDEPAEGFPMLKVKDIKSAYSHWQESKPILEKMVKQTRASSGVIWNSFKELEESELETVQREIPAPSFLIPLPKHYRASSTSLLDTDRSVFQWLDQQPPGSVLYVGFGSQSEVDRATFLAIARGLVASGLSFLWLVRPGFVKGETWIESLPPGFLGPRGRVVKSAPQQEVLAHGAIGAFWTHGGWNGTMEAVCEGVPMIFSDFGLDQPLNARYMSDVLHVGVYLENGWDPEEIARAIRLVMVDPEGAPIRENARVLKQKADRSIMKGGSSYESLESLVSYISSL

SEQ ID NO: 288 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADEFRKLLEEEIAASESDEEVSCLITDALWYFAQDVADSLNLRRLVLQTGSLFNFHCLVSLPKFDELGYLDPDNKKDLDKPAEGFPMLKVKDIKRAYSHWQESKPILEKMVKQTKASSGVIWNSFKELEESELETVWREIPAPSFLIPLPKHYRASSTSLLDTDRSVFQWLDQQPPRSVLYVGFGSQSEVDPADFLAIARGLVESGISFLWLVRPGFVKGETWIEVLPDGFLGERGRIVKSAPQQEVLAHGAIGAFWTHGGWNGTMESVCEGVPMIFSDFGLDQPLNARYMSEVLKVGVYLENGWEPGRIAAAIRLVMVDPEGAYIRENARVLKQKADRSIMKGGSSYESLESLVSYISSL

SEQ ID NO: 289 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEPFRKELEKLMLASESDEEVSCLITDALWYFAQDVADSLNLKRLVLQTGSLFNFHCLVSLPKFDELGYLDPDDKTRLDEPVEGFPELKVKDIKSAYSHWQESKPILDKMVKQTRASSGVIWNSFKELEESELETVQREIPAPSFLIPLPKHYRASSTSLLDTDRSVFQWLDQQPPRSVLYVGFGSQSEVDPEDFLKIARGLVDSKISFLWLVRPGFVKGYEWIEPLPEGFLGERGRIVKSAPQQEVLAHGAIGAFWTHGGWNGTMESVCEGVPMIFSDFGLDQPLNARYMSKVLKVGVYLENGWDPERIANAIRLVMVDEEGKEIRQNAEVLKQKADRSIMKGGSSYESLESLVSYISSL

SEQ ID NO: 290 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEPFRKELEELISASESDEEVSCLITDALWYFAQEVADSLNLKRLVLQTGSLFNFHCLVSLPKFDELGYLDPDNKTRLDEPVEGFPWLKVKDIKSAYSHWQESKPILDKMVKQTRASSGVIWNSFKELEESELETVQREIPAPSFLIPLPKHYRASSTSLLDTDRSVFQWLDQQPPRSVLYVGFGSQSEVDPKDFLAIAEGLVASGVSFLWLVRPGFVKGEEWIEVLPEGFLGERGRIVKSAPQQEVLAHGAIGAFWTHGGWNGTMESVCEGVPMIFSDFGLDQPLNARYMSDVLKVGVYLENGWEPGAIAEAIRLVMVDERGEVIRRNARVLKQKADRSIMKGGSSYESLESLVSYISSL

SEQ ID NO: 291 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGADRFRKELELEMSASESDEEVSCLITDALWYFAQDVADSLNLKRLVLQTGSLFNFHCLVSLPKFDELGYLDPDNKKDLDKPVEGFPELKVKDIKSAYSHWQESKPILEKMVKQTRASSGVIWNSFKELEESELETVQREIPAPSFLIPLPKHYRASSTSLLDTDRSVFQWLDQQPPRSVLYVGFGSQSEVDPEDFLKIAEGLVKSGISFLWLVRPGFVKGYTWIEPLPDGFLGEKGRVVKSAPQQEVLAHGAIGAFWTHGGWNGTMEAVCEGVPMIFSDFGLDQPLNARYMSNVLHVGVYLENGWEPEEIAKAIRLVMVDPEGEPMRQNARVLKQKADRSIMKGGSSYESLESLVSYISSL

SEQ ID NO: 292 - protein, 457 aa, synthetic construct
MPNTNTTVRRTRRIILFPVPFPGHWNPMLQLANVLYSKGFSITIFHTNFNKPKTSNYPHFTFRFILDNDPQDEWLRNLPTHGPGVGARIPVINKHGADEFRKELEELILASESDEEVSCLITDALWYFAQSVADSLNLKRLVLQTGSLFNFHCLVSLPKFDELGYLDPDNKTDLDKPAPGFPWLKVKDIKSAYSHWQESKPILMKMVKQTRASSGVIWNSFKELEESELETVQREIPAPSFLIPLPKHYRASSTSLLDTDRSVFQWLDQQPPGSVLYVGFGSQSEVDPEDFLRIARGLVLSKLSFLWLVRPGFVKGHTWIEPLPEGFLGERGRVVKSAPQQEVLAHGAIGAFWTHGGWNGTMESVCEGVPMIFSDFGLDQPLNARYMSEVLHVGVYLENGWEPEEIARAIRLVMVDPEGEYIRQNARVLKQKADRSIMKGGSSYESLESLVSYISSL

SEQ ID NO: 293 - protein, 457 aa, synthetic construct
MPNTNTTVRRRRRIILFPVPFPGHLNPMLQLANVLYSKGFSITIFHTNFNKPKTSNYPHFTFIFILDNDPQDEWLRNLPTHGPGVGARIPVINKHGAEEFRKELENLMNASESDEEVSCLITDALWYFAQPVADSLNLKRLVLQTGSLFNFHCLVSLPKFDELGYLDPDDKTRLDEPVEGFPMLKVKDIKSAYSHWQESKPILEKMVKQTRESSGVIWNSFKELEESELETVQREIPAPSFLIPLPKHYRASSTSLLDTDRSVFQWLDQQPPGSVLYVGFGSQSEVDPADFLRIAEGLVRSGLPFLWLVRPGFVKGYTWIEPLPEGFLGERGRVVKSAPQQEVLAHPAIGAFWTHGGWNGTMEAVCEGVPMIFSDFGLDQPLNARYMSDVLHVGVYLENGWEPEEIARAIRLVMVDPEGEPIRQNARVLKQKADRSIMKGGSSYESLESLVSYISSL

SEQ ID NO: 294 - protein, 457 aa, synthetic construct
MPNTNTTVRRTRRIVLFPVPFPGHWNPMLQLANVLYSKGFSITIFHTNFNKPKTSNYPHFTFRFILDNDPQDEWLRNLPTHGPGVGARIPVINKHGKEEFRKELELLMEASESDEEVSCLITDALWYFAQEVADSLNLKRLVLQTGSLFNFHCLVSLPKFDELGYLDPDDKTRLEEPVEGFPMLKVKDIKSAYSHWQESKPILMKMVKQTRASSGVIWNSFKELEESELETVQREIPAPSFLIPLPKHYRASSTSLLDTDRSVFQWLDQQPPGSVLYVGFGSQSEVDPATFLAIARGLVASGLPFLWLVRPGFVKGHTWIEPLPPGFLGERGRVVKSAPQQEVLAHPAIGAFWTHGGWNGTMESVCEGVPMIFSDFGLDQPLNARYMSEVLHVGVYLENGWDPGEIARAIRLVMVDEAGEPMRQNARVLKQKADRSIMKGGSSYESLESLVSYISSL

SEQ ID NO: 295 - protein, 457 aa, synthetic construct
MPNTNTTVRRTRRIVLFPVPFPGHLNPMLQLANVLYSKGFSITIFHTNFNKPKTSNYPHFTFRFILDNDPQDEWLRNLPTHGPGVGARIPVINKHGADEFRKALEELMEASESDEEVSCLITDALWYFAQPVADSLNLKRLVLQTGSLFNFHCLVSLPKFDELGYLDPDDKTDLDKPVEGFPELKVKDIKSAYSHWQESKPILEKMVKQTRESSGVIWNSFKELEESELETVQREIPAPSFLIPLPKHYRASSTSLLDTDRSVFQWLDQQPPGSVLYVGFGSQSEVDPATFLTIARGLVLSGQRFLWLVRPGFVKGSTWIEPLPEGFLGEKGRVVKSAPQQEVLAHGAIGAFWTHGGWNGTMEAVCEGVPMIFSDFGLDQPLNARYMSDVLHVGVYLENGWDPEAIAEAIRRVMVDEEGEYIRQNARVLKQKADRSIMKGGSSYESLESLVSYISSL

SEQ ID NO: 296 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGAEEFRKTLEELMNASESDEEVSCLITDALWYFAQEVADSLNLKRLVLQTGSLFNFHCLVSLPKFDELGYLDPDNKKDLEKPAEGFPMLKVKDIKSAYSHWQESKPILDKMVKQTRASSGVIWNSFKELEESELETVQREIPAPSFLIPLPKHYRASSTSLLDTDRSVFQWLDQQPPRSVLYVGFGSQSEVDPKDFLAIAEGLVRSGLPFLWLVRPGFVKGYEWIEPLPDGFLGEKGRIVKSAPQQEVLAHPAIGAFWTHGGWNGTMESVCEGVPMIFSDFGLDQPLNARYMSDVLHVGVYLENGWDPERIANAIRRVMVDPEGEVIRQNARVLKQKADRSIMKGGSSYESLESLVSYISSL

SEQ ID NO: 297 - protein, 457 aa, synthetic construct
MPNTNTTVRRTRRIILFPVPFPGHLNPMLQLANVLYSKGFSITIFHTNFNKPKTSNYPHFTFRFILDNDPQDEWLRNLPTHGPGVGARIPVINKHGADEFRKELELLILASESDEEVSCLITDALWYFAQPVADSLNLKRLVLQTGSLFNFHCLVSLPKFDELGYLDPDDKTRLDEPVVGFPQLKVKDIKSAYSHWQESKPILEKMVKQTRASSGVIWNSFKELEESELETVQREIPAPSFLIPLPKHYRASSTSLLDTDRSVFQWLDQQPPGSVLYVGFGSQSEVDPKTFLEIARGLVASGLPFLWLVRPGFVKGETWIEVLPEGFLGEKGRVVKSAPQQEVLAHPAIGAFWTHGGWNGTMEAVCEGVPMIFSDFGLDQPLNARYMSDVLHVGVYLENGWDPEAIAAAIRLVMVDPAGAPIRQNAAVLKQKADRSIMKGGSSYESLESLVSYISSL

SEQ ID NO: 298 - protein, 457 aa, synthetic construct
MPNTNTTVRRRRRIILFPVPFPGHWNPMLQLANVLYSKGFSITIFHTNFNKPKTSNYPHFTFRFILDNDPQDEWLRNLPTHGPGVGARIPVINKHGAERFKKELEELISASESDEEVSCLITDALWYFAQEVADSLNLKRLVLQTGSLFNFHCLVSLPKFDELGYLDPDNKTRLEEPAEGFPELKVKDIKSAYSHWQESKPILMKMVKQTRASSGVIWNSFKELEESELETVQREIPAPSFLIPLPKHYRASSTSLLDTDRSVFQWLDQQPPRSVLYVGFGSQSEVDPKTFLEIARGLVLSKLPFLWLVRPGFVKGYTWIEPLPEGFLGERGRVVKSAPQQEVLAHPAIGAFWTHGGWNGTMESVCEGVPMIFSDFGLDQPLNARYMSEVLKVGVYLDNGWEPERIAEAIRRVMVDPEGEPIRQNARVLKQKADRSIMKGGSSYESLESLVSYISSL

SEQ ID NO: 299 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGADEFRKKLEELISASESDEEVSCLITDALWYFAQDVADSLNLRRLVLQTGSLFNFHCLVSLPKFDELGYLDPDDKTRLDEPAEGFPMLKVKDIKRAYSHWQESKPILEKMVKQTKASSGVIWNSFKELEESELETVWREIPAPSFLIPLPKHYRASSTSLLDTDRSVFQWLDQQPPESVLYVGFGSQSEVDPETFLEIAEGLVASGQPFLWLVRPGFVKGETWIEVLPEGFLGERGRIVKSAPQQEVLAHPAIGAFWTHGGWNGTMESVCEGVPMIFSDFGLDQPLNARYMSEVLKVGVYLDNGWERERIARAIRLVMVDPEGEYIRQNARVLKQKADRSIMKGGSSYESLESLVSYISSL

SEQ ID NO: 300 - protein, 457 aa, synthetic construct
MPNTNTTVRRRRRIILFPVPFPGHLNPMLQLANVLYSKGFSITILHTNFNKPKTSNYPHFTFVFILDNDPQDEWLRNLPTHGPGVGARIPVINKHGKEPLRKELEKLIEASESDEEVSCLITDALWYFAQEVADSLNLRRLVLQTGSLFNFHCLVSLPKFDELGYLDPDDKSRLDEPAEGFPWLKVKDIKRAYSHWQESKPILEKMVKQTKESSGVIWNSFKELEESELETVWREIPAPSFLIPLPKHYRASSTSLLDTDRSVFAWLDQQPPGSVLYVGFGSQSEVDPKTFLEIARGLVLSGLPFLWLVRPGFVKGETWIEVLPDGFLGEKGRIVKSAPQQEVLAHPAIGAFWTHGGWNGTMESVCEGVPMIFSDFGLDQPLNARYMSEVLHVGVYLDNGWDPEEIARAIRLVMVDPEGAPIRENARVLKQKADRSIMKGGSSYESLESLVSYISSL

SEQ ID NO: 301 - protein, 457 aa, synthetic construct
MPNTNTTVRRRRRIILFPVPFPGHLNPMLQLANVLYSKGFSITILHTEFNKPKTSNYPHFTFRTILDNDPQDEWLRNLPTHGPGVGARIPVINKHGAERLRKELEEIISASESDEEVSCLITDALWYFAQEVADSLNLKRLVLQTGSLFNFHCLVSLPKFEELGYLDPDDKTRLDEPAEGFPELKVKDIKSAYSHWQESKPILEKMVKQTRASSGVIWNSFKELEESELETVQREIPAPSFLIPLPKHYRASSTSLLDTDRSVFDWLDQQPPESVLYVGFGSQSEVEPEDFLKIARGLVDSKVPFLWLVRPGFVKGHEWIEPLPEGFLGEKGRVVKSAPQQEVLAHPAIGAFWTHGGWNGTMEAVCEGVPMIFSDFGLDQPLNARYMSDVKKIGVYLDNGWDPAAIANAIRRVMVDPEGEPIRQNAKVLKQKADRSIMKGGSSYESLESLVSYISSL

SEQ ID NO: 302 - protein, 457 aa, synthetic construct
MPNTNTTVRRTRRIVLFPVPFPGHLNPMLQLANVLYSKGFSITILHTNFNKPKTSNYPHFTFIFILDNDPQDEWLRNLPTHGPGVGARIPVINKHGAEELRKTLEELIEASESDEEVSCLITDALWYFAQPVADSLNLKRLVLQTGSLFNFHCLVSLPKFDELGYLDPDDKTDLDKPVEGFPQLKVKDIKSAYSHWQESKPILEKMVKQTRASSGVIWNSFKELEESELETVQREIPAPSFLIPLPKHYRASSTSLLDTDRSVFAWLDQQPPSSVLYVGFGSQSEVDPEDFLKIARGLVASGLPFLWLVRPGFVKGYTWIEPLPEGFLGPKGRVVKSAPQQEVLAHPAIGAFWTHGGWNGTMEAVCEGVPMIFSDFGLDQPLNARYMSDVLHVGVYLDEGWDPERIAAAIRLVMVDEEGAYIRENARVLKQKADRSIMKGGSSYESLESLVSYISSL

SEQ ID NO: 303 - protein, 457 aa, synthetic construct
MPNTNTTVRRRRRIILFPVPFPGHLNPMLQLANVLYSKGFSITILHTEFNKPKTSNYPHFTFRTILDNDPQDEWLRNLPTHGPGVGARIPVINKHGADRLKKLLEELISASESDEEVSCLITDALWYFAQEVADSLNLKRLVLQTGSLFNFHCLVSLPHFEELGYLDPSDTTRLDEPVVGFPELLVKDIKSAYSHWQESKPILEKMVKQTRASSGVIWNSFKELEESELETVQREIPAPSFLIPLPKHYRASSTSLLDTDRSVFDWLDQQPPSSVLYVGFGSQSEVEPKDFLAIAEGLVRSGLPFLWLVRPGFVKGYEWIEPLPDGFLGERGRVVKSAPQQEVLAHPAIGAFWTHGGWNGTMEAVCEGVPMIFSDFGLDQPLNARYMSDVRKIGVYLRDGWDPERIAAAIRRVMVDPEGEPIRQNARVLKQKADRSIMKGGSSYESLESLVSYISSL

SEQ ID NO: 304 - protein, 457 aa, synthetic construct
MPNTNTTVRRRRRIVLFPVPFPGHLNPMLQLANVLYSKGFSITILHTSFNKPKTSNYPHFTFVEILDNDPQDEWLRNLPTHGPGVGARIPVINKHGAERFRKILEELISASESDEEVSCLITDALWYFAKDVADSLNLKRLVLQTGSLFNFHCLVSLPKFDELGYLDPDNKTDLDKPAEGFPELKVKDIKSAYSHWQESKPILEKMVKQTRASSGVIWNSFKELEESELETVQREIPAPSFLIPLPKHYRASSTSLLDTDRSVFDWLDQQPPRSVLYVGFGSQSEVEPEEFLEIARGLVLSGQPFLWLVRPGFVKGYTWIEPLPEGFLGPAGRVVKSAPQQEVLAHPAIGAFWTHGGWNGTMEAVCEGVPMIFSDFGLDQPLNARYMSDVLHVGVYLDNGWDPERIAAAIRLVMVDPEGEPIRQNARVLKQKADRSIMKGGSSYESLESLVSYISSL

SEQ ID NO: 305 - protein, 457 aa, synthetic construct
MPNTNTTVRRRRRIILFPVPFPGHWNPMLQLANVLYSKGFSITILHTNFNKPKTSNYPHFTFRTILDNDPQDEWLRNLPTHGPGVGARIPVINKHGKDRFRKLLEEIISASESDEEVSCLITDALWYFAQEVADSLNLKRLVLQTGSLFNFHCLVSLPKFDELGYLDPSNNKDLEKPAEGFPMLKVKDIKSAYSHWQESKPILMKMVKQTRASSGVIWNSFKELEESELETVQREIPAPSFLIPLPKHYRASSTSLLDTDRSVFAWLDQQPPRSVLYVGFGSQSEVEPKEFLEIARGLVESGLPFLWLVRPGFVKGYEWIEPLPEGFLGEKGRVVKSAPQQEVLAHPAIGAFWTHGGWNGTMESVCEGVPMIFSDFGLDQPLNARYMSEVKHIGVYLDNGWEPGEIARAIRLVMVDPEGEPMRQNARVLKQKADRSIMKGGSSYESLESLVSYISSL

SEQ ID NO: 306 - protein, 457 aa, synthetic construct
MPNTNTTVRRRRRIVLFPVPFPGHLNPMLQLANVLYSKGFSITILHTNFNKPKTSNYPHFTFVTILDNDPQDEWLRNLPTHGPGVGARIPVINKHGADRFRQILEELISASESDEEVSCLITDALWYFAQEVADSLNLRRLVLQTGSLFNFHCLVSLPKFEELGYLDPNDKTRLEEPAEGFPMLKVKDIKRAYSHWQESKPILEKMVKQTKASSGVIWNSFKELEESELETVWREIPAPSFLIPLPKHYRASSTSLLDTDRSVFAWLDQQPPRSVLYVGFGSQSEVEPEDFLKIARGLVLSGQPFLWLVRPGFVKGYEWIEPLPDGFLGPQGRIVKSAPQQEVLAHGAIGAFWTHGGWNGTMESVCEGVPMIFSDFGLDQPLNARYMSEVRKIGVYLDNGWEPERIANAIRLVMVDPRGEPMRQNARVLKQKADRSIMKGGSSYESLESLVSYISSL

SEQ ID NO: 307 - protein, 457 aa, synthetic construct
MPNTNTTVRRRRRIALFPVPFPGHLNPMLQLANVLYSKGFSITIFHTNFNKPKTSNYPHFTFEFILDNDPQDEWLRNLPTHGPGVGARIPVINKHGKDRFKELLENLIAASESDETLSCLITDALWYFAQEVADSLNLRRLVLQTGSLFNFHCLVSLPKFDELGYLDPDNKTDLDKPAEGFPMLKVKDIKRAYSHWQESKPILEKMVKQTKASSGVIWNSFKELEESELETVWREIPAPSFLIPLPKHYRASSTSLLDTDRSVFQWLDQQPPRSVLYVGFGSQSEVDPATFLAIAEGLVRSGQPFLWLVRPGFVKGHTWIEPLPEGFLGPQGRIVKSAPQQEVLAHPAIGAFWTHGGWNGTMESVCEGVPMIFSDFGLDQPLNARYMSEVLKVGVYLDEGWDPARIAEAIRLVMVDPEGLPIRENARVLKQKADRSIMKGGSSYESLESLVSYISSL

SEQ ID NO: 308 - protein, 457 aa, synthetic construct
MPNTNTTVRRRRTIILFPVPFPGHWNPMLQLANVLYSKGFKIIILHTNFNKPKTSNYPHFTFVTILDNDPQDEWLRNLPTHGPGVGARIPVINKHGKDRFKKILEEIISASESDEEVSCLITDALWYFTQDVADELNLKRLVLQTGSLFNFHCLVSLPKFEELGLLDPDDKTRLDEPAPGFPELLVKDIKSAYSHWQESKPILMKMVKQTRASSGVIWNSFKELEESELETVQREIPAPSFLIPLPKHYRASSTSLLDTDRSVFDWLDQQPPRSVLYVGFGSQSEVEPEDFLAIARGLVLSGLPFLWLVRPGFVKGHEWIEVLPPGFLGPLGRVVKSAPQQEVLAHPAIGAFWTHGGWNGTMESVCEGVPMIFSDFGLDQPLNARYMSEVKKIGVYLRDGWDPERIAAAIRLVMVDERGEPIRQNARELKQKADRSIMKGGSSYESLESLVSYISSL

SEQ ID NO: 309 - protein, 457 aa, synthetic construct
MPNTNTTVRRRRRIILFPVPFPGHLNPMLQLANVLYSKGFSITILHTAFNAPKTSNYPHFTFRTILDNDPQDEWLRNLPTHGPGVGARIPVINKHGADELRKTLQEIMEASESDEEVSCLITDALWYFAQPVADSLNLKRLVLQTGSLFNFHCLVSLPKFEELGYLDPDDKSRLEEPVVGFPELKVKDIKSAYSHWQESKPILDKMVKQTRASSGVIWNSFKELEESELETVQREIPAPSFLIPLPKHYRASSTSLLDTDRSVFAWLDQQPPGSVLYVGFGSQSEVEPKEFLEIARGLVKSGLPFLWLVRPGFVKGHEWIEVLPDGFLGEKGRIVKSAPQQEVLAHPAIGAFWTHGGWNGTMESVCEGVPMIFSDFGLDQPLNARYMSDVRHVGVYLDNGWDPERIANAIRRVMVDEEGAVIRQNARELKQKADRSIMKGGSSYESLESLVSYISSL

SEQ ID NO: 310 - protein, 457 aa, synthetic construct
MPNTNTTVRRRRRIVLFPVPFPGHLNPMLQLANVLYSKGFSITILHTRFNAPKTSNYPHFTFRFILDNDPQDEWLRNLPTHGPGVGARIPVINKHGAEEFKKELEKLISASESDEEVSCLITDALWYFTQDVAKSLNLKRLVLQTGSLFNFHCLVSLPKFEELGYLDPSDKTRLDEPVEGFPELKVKDIKSAYSHWQESKPILDKMVKQTRESSGVIWNSFKELEESELETVQREIPAPSFLIPLPKHYRASSTSLLDTDRSVFDWLDQQPPESVLYVGFGSQSEVEPKDFLAIAEGLVRSKLSFLWLVRPGFVKGEEWIEVLPEGFLGERGRIVKSAPQQEVLAHPAIGAFWTHGGWNGTMESVCEGVPMIFSDFGLDQPLNARYMSDVKKVGVYLENGWNPEEIAAAIRRVMVDPEGEVIRKNARELKQKADRSIMKGGSSYESLESLVSYISSL

SEQ ID NO: 311 - protein, 457 aa, synthetic construct
MPNTNTTVRRTRRIVLFPVPFPGHLNPMLQLANVLYSKGFSITILHTRFNAPKTSNYPHFTFITILDNDPQDEWLRNLPTHGPGVGARIPVINKHGADEFRKTLEDLISASESDEEVSCLITDALWYFAQPVADSLNLKRLVLQTGSLFNFHCLVSLPKFEELGILDPDDKTRLDEPVPGFPQLLVKDIKSAYSHWQESKPILEKMVKQTRASSGVIWNSFKELEESELETVQREIPAPSFLIPLPKHYRASSTSLLDTDRSVFAWLDQQPPESVLYVGFGSQSEVEPKEFLEIAEGLVASGLSFLWLVRPGFVKGYTWIEPLPDGFLGERGRVVKSAPQQEVLAHPAIGAFWTHGGWNGTMEAVCEGVPMIFSDFGLDQPLNARYMSDVKHVGVYLDNGWERGEIAAAIRRVMVDPEGEPIRQNARELKQKADRSIMKGGSSYESLESLVSYISSL

SEQ ID NO: 312 - protein, 457 aa, synthetic construct
MPNTNTTVRRRRRIVLFPVPFPGHLNPMLQLANVLYSKGFKITILHTNFNAPKTSNYPHFTFIFILDNDPQDEWLRNLPTHGPGVGARIPVINKHGKDEFKKTLEDLISASESDEEVSCLITDALWYFAQDVADSLNLKRLVLQTGSLFNFHCLVSLPKFEELGLLDPDDKTDLDKPVPGFPWLKVKDIKSAYSHWQESKPILDKMVKQTRASSGVIWNSFKELEESELETVQREIPAPSFLIPLPKHYRASSTSLLDTDRSVFQWLDQQPPGSVLYVGFGSQSEVEPEEFLEIARGLVESGLPFLWLVRPGFVKGYEWIEPLPDGFLGEKGRIVKSAPQQEVLAHPAIGAFWTHGGWNGTMESVCEGVPMIFSDFGLDQPLNARYMSDVRHVGVYLENGWEPGEIARAIRLVMVDEEGEVMRQNARLLKQKADRSIMKGGSSYESLESLVSYISSL

SEQ ID NO: 313 - protein, 457 aa, synthetic construct
MPNTNTTVRRTRRIILFPVPFPGHLNPMLQLANVLYSKGFSITIFHTQFNAPKTSNYPHFTFRTILDNDPQDEWLRNLPTHGPGVGARIPVINKHGADRFRKELEELIEASESDEEVSCLITDALWYFAQPVADSLNLRRLVLQTGSLFNFHCLVSLPKFEELGILDPDDKSRLEEPVPGFPMLKVKDIKRAYSHWQESKPILEKMVKQTKESSGVIWNSFKELEESELETVWREIPAPSFLIPLPKHYRASSTSLLDTDRSVFDWLDQQPPGSVLYVGFGSQSEVEPADFLAIARGLVDSGLPFLWLVRPGFVKGYTWIEPLPDGFLGEKGRIVKSAPQQEVLAHPAIGAFWTHGGWNGTMESVCEGVPMIFSDFGLDQPLNARYMSEVRHVGVYLDNGWDPAEIAAAIRRVMVDPEGEPIRQNARELKQKADRSIMKGGSSYESLESLVSYISSL

SEQ ID NO: 314 - protein, 457 aa, synthetic construct
MPNTNTTVRRRRRIILFPVPFPGHLNPMLQLANVLYSKGFSITILHTRFNAPKTSNYPHFTFRFILDNDPQDEWLRNLPTHGPGVGARIPVINKHGAEEFRKELELIMLASESDEEVSCLITDALWYFTQEVADSLNLKRLVLQTGSLFNFHCLVSLPKFEELGYLDPDDKTRLDEPAEGFPQLKVKDIKSAYSHWQESKPILDKMVKQTRASSGVIWNSFKELEESELETVQREIPAPSFLIPLPKHYRASSTSLLDTDRSVFQWLDQQPPKSVLYVGFGSQSEVEPEDFLAIARGLVLSGQPFLWLVRPGFVKGHEWIEPLPDGFLGEKGRIVKSAPQQEVLAHGAIGAFWTHGGWNGTMESVCEGVPMIFSDFGLDQPLNARYMSDVKKIGVYLDRGWEPERIAEAIRLVMVDERGKVIRQNALELKQKADRSIMKGGSSYESLESLVSYISSL

SEQ ID NO: 315 - protein, 457 aa, synthetic construct
MPNTNTTVRRRRRIILFPVPFPGHLNPMLQLANVLYSKGFSITILHTRFNAPKTSNYPHFTFRFILDNDPQDEWLRNLPTHGPGVGARIPVINKHGAAPLRAELEKLMAASESDEEVSCLITDALWYFAQDVADSLNLKRLVLQTGSLFNFHCLVSLPKFDELGLLDPDDKTRLDEPVPGFPELKVKDIKSAYSHWQESKPILDKMVKQTRASSGVIWNSFKELEESELETVQREIPAPSFLIPLPKHYRASSTSLLDTDRSVFDWLDQQPPSSVLYVGFGSQSEVDPKTALEIAEGLVKSGIPFLWLVRPGFVKGYEWIEILPEGFLGEKGRIVKSAPQQEVLAHPAIGAFWTHGGWNGTMESVCEGVPMIFSDFGLDQPLNARYMSDVLHIGVYLDNGWNPEEIANAIRLVMVDPEGEVIRQNARELKQKADRSIMKGGSSYESLESLVSYISSL

SEQ ID NO: 316 - protein, 457 aa, synthetic construct
MPNTNTTVRRRRRIVLFPVPFPGHWNPMLQLANVLYSKGFSITILHTNFNKPKTSNYPHFTFITILDNDPQDEWLRNLPTHGPGVGARIPVINKHGAERFRKILEELMLASESDEEISCLITDALWYFTQDVADSLNLKRLVLQTGSLFNFHCLVSLPKFEELGLLDPSDKTRLEEPVPGFPWLKVKDIKSAYSHWQESKPILMKMVKQTRASSGVIWNSFKELEESELETVQREIPAPSFLIPLPKHYRASSTSLLDTDRSVFEWLDQQPPRSVLYVGFGSQSEVEPEDFLRIAEGLVRSGLPFLWLVRPGFVKGHEWIEPLPPGFLGEKGRVVKSAPQQEVLAHGAIGAFWTHGGWNGTMESVCEGVPMIFSDFGLDQPLNARYMSEVKKVGVYLDNGWEPGRIAEAIRLVMVDEEGRPIRQNAEELKQKADRSIMKGGSSYESLESLVSYISSL

SEQ ID NO: 317 - protein, 457 aa, synthetic construct
MPNTNTTVRRRRRIILFPVPFPGHWNPMLQLANVLYSKGFSITILHTNFNKPKTSNYPHFTFVEILDNDPQDEWLRNLPTHGPGVGARIPVINKHGADRLKEELEKLMLASESDEEISCLITDALWYFAQSVADSLNLKRLVLQTGSLFNFHCLVSLPKFDELGYLDPNNKTRLEEPAEGFPELLVKDIKSAYSHWQESKPILMKMVKQTRESSGVIWNSFKELEESELETVQREIPAPSFLIPLPKHYRASSTSLLDTDRSVFDWLDQQPPESVLYVGFGSQSEVEPKDFLTIAEGLVASKQPFLWLVRPGFVKGSTWIEPLPPGFLGERGRVVKSAPQQEVLAHPAIGAFWTHGGWNGTMESVCEGVPMIFSDFGLDQPLNARYMSRVRHIGVYLDNGWDRRRIAEAIRLVMVDERGRPIRENARVLKQKADRSIMKGGSSYESLESLVSYISSL

SEQ ID NO: 318 - protein, 457 aa, synthetic construct
MPNTNTTVRRRRTIILFPVPFPGHWNPMLQLANVLYSKGFKIIILHTKFNKPKTSNYPHFTFIEILDNDPQDEWLRNLPTHGPGVGARIPVINKHGADEFRKTLEEIILASESDEEVSCLITDALWYFAQSVADSLNLKRLVLQTGSLFNFHCLVSLPKFEELGYLDPDDKSRLDEPVVGFPWLKVKDIKSAYSHWQESKPILMKMVKQTRESSGVIWNSFKELEESELETVQREIPAPSFLIPLPKHYRASSTSLLDTDRSVFDWLDRQPPGSVLYVGFGSQSEVEPEEFLAIAEGLRRSGLPFLWLVRPGFVKGHTWIEVLPEGFLGERGRVVKSAPQQEVLAHPAIGAFWTHGGWNGTMESVCEGVPMIFSDFGLDQPLNARYMSEVRHVGVYLDNGWEPEEIAAAIRLVMVDPAGEPIRQNARELKQKADRSIMKGGSSYESLESLVSYISSL

SEQ ID NO: 319 - protein, 457 aa, synthetic construct
MPNTNTTVRRRRRIILFPVPFPGHLNPMLQLANVLYSKGFSITILHTNFNAPKTSNYPHFTFVFILDNDPQDEWLRNLPTHGPGVGARIPVINKHGAERLRQTLELLMQASESDEEISCLITDALWYFAQEVADSLNLRRLVLQTGSLFNFHCLVSLPKFEELGYLDPDNQTDLEKPAVGFPMLKVKDIKRAYSHWQESKPILEKMVKQTKASSGVIWNSFKELEESELETVWREIPAPSFLIPLPKHYRASSTSLLDTDRSVFDWLDQQPPRSVLYVGFGSQSEVEPEEFLEIAEGLVASGQSFLWLVRPGFVKGSEWIEPLPEGFFGERGRIVKSAPQQEVLAHPAIGAFWTHGGWNGTMESVCEGVPMIFSDFGLDQPLNARYMSEVRHIGVYLEDGWDRERIARAIRLVMVDPEGEPIRQNARVLKQKADRSIMKGGSSYESLESLVSYISSL

SEQ ID NO: 320 - protein, 457 aa, synthetic construct
MPNTNTTVRRRRRIVLFPVPFPGHLNPMLQLANVLYSKGFSITILHTNFNAPKTSNYPHFTFRFILDNDPQDEWLRNLPTHGPGVGARIPVINKHGADRLRKELELLKLASESDEEISCLITDALWYFAQSVAESLNLKRLVLQTGSLFNFHCLVSLPEFEKLGLLDPSNNTNLDEPVPGFPMLKVKDIKSAYSHWQESKPILEKMVKQTRASSGVIWNSFKELEESELETVQREIPAPSFLIPLPKHYRASSTSLLDTDRSVFAWLDQQPPRSVLYVGFGSQSEVEPEDFLAIARGLVLSGQPFLWLVRPGFVKGSTWIEPLPEGFLGERGRVVKSAPQQEVLAHPAIGAFWTHGGWNGTMEAVCEGVPMIFSDFGLDQPLNARYMSDVLKVGVYLDNGWEPEAIARAIRLVMVDPEGEYIRQNARELKQKADRSIMKGGSSYESLESLVSYISSL

SEQ ID NO: 321 - protein, 457 aa, synthetic construct
MPNTNTTVRRRRTIILFPVPFPGHLNPMLQLANVLYSKGFKIIILHTNFNKPKTSNYPHFTFVTILDNDPQDEWLRNLPTHGPGVGARIPVINKHGADELKKTLENIIKASESDEEVSCLITDALWYFTQSVADSLNLKRLVLQTGSLFNFHCLVSLPKFEELGYLDPSNTTDLDKPVEGFPMLLVKDIKSAYSHWQESKPILDKMVKQTRLSSGVIWNSFKELEESELETVQREIPAPSFLIPLPKHYRASSTSLLDTDRSVFEWLDQQPERSVLYVGFGSQSEVEPEDFLAIARGLVLSGQPFLWLVRPGFVKGYEWIEPLPEGFLGEKGRIVKSAPQQEVLAHPAIGAFWTHGGWNGTMESVCEGVPMIFSDFGLDQPLNARYMSDVLKIGVYLDRGWEPERIAEAIRRVMVDPEGEVIRKNARELKQKADRSIMKGGSSYESLESLVSYISSL

SEQ ID NO: 322 - protein, 457 aa, synthetic construct
MPNTNTTVRRRRTIVLFPVPFPGHLNPMLQLANVLYSKGFNIIIFHTRFNAPKTSNYPHFTFKFILDNDPQDEWLRNLPTHGPGVGARIPVINKHGAEEFRKTLEELISASESDEEISCLITDALWYFTYDVAKSLNLKRLVLQTGSLFNFHCLVSLPKFEELGYLDPSNTTDLDKPVVGAPWLKVKDIKSAYSHWQESKPILDKMVKQTRLSSGVIWNSFKELEESELETVQREIPAPSFLIPLPKHYRASSTSLLDTDRSVFEWLDQQPPESVLYVGFGSQSEVEPEEFLAIAEGLRRSGLPFLWLVRPGFVKGHEWIEPLPEGFLGPLGRIVKSAPQQEVLAHPAIGAFWTHGGWNGTMESVCEGVPMIFSDFGLDQPLNARYMSDVLKIGVYLDNGWEPERIAAAIRRVMVDPEGAGMRENARVLKQKADRSIMKGGSSYESLESLVSYISSL

SEQ ID NO: 323 - protein, 457 aa, synthetic construct
MPNTNTTVRRRRTIILFPVPFPGHWNPMLQLANVLYSKGFKIIILHTNFNKPKTSNYPHFTFVEILDNDPQDEWLRNLPTHGPGVGARIPVINKHGKERFKKILEEIISASESDEEVSCLITDALWYFTQEVADSLNLKRLVLQTGSLFNFHCLVSLPKFEELGYLDPSNQKDLDKPAEGFPWLKVKDIKSAYSHWQESKPILMKMVKQTRASSGVIWNSFKELEESELETVQREIPAPSFLIPLPKHYRASSTSLLDTDRSVFDWLDGQPPRSVLYVGFGSQSEVDPKTALAIAEGLKLSGIPFLWLVRPGFVKGHEWIEVLPEGFLGEKGRVVKSAPQQEVLAHPAIGAFWTHGGWNGTMESVCEGVPMIFSDFGLDQPLNARYMANVRHVGVYLDNGWDPAAIAAAIRRVMVDEEGAPIRQNALELKQKADRSIMKGGSSYESLESLVSYISSL

SEQ ID NO: 324 - protein, 457 aa, synthetic construct
MPNTNTTVRRRRRIILFPVPFPGHWNPMLQLANVLYSKGFSITIFHTNFNKPKTSNYPHFTFVEILDNDPQDEWLRNLPTHGPGVGARIPVINKHGADRFKEILEKIISASESDEEVSCLITDALWYFTNSVAKSLNLKRLVLQTGSLFNFHCLVSLPKFDELGLLDPSNTTGLDEPVPGAPWLKVKDIKSAYSHWQESKPILMKMVKQTRESSGVIWNSFKELEESELETVQREIPAPSFLIPLPKHYRASSTSLLDTDRSVFQWLDQQPPRSVLYVGFGSQSEVEPEDALRIARGLVDSGVSFLWLVRPGFVKGHEWIEVLPEGFLGERGRVVKSAPQQEVLAHPAIGAFWTHGGWNGTMESVCEGVPMIFSDFGLDQPLNARYMAEVKKIGVYLDDGWDPARIAAAIRLVMVDPEGEPIRQNALELKQKADRSIMKGGSSYESLESLVSYISSL

SEQ ID NO: 325 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEEFREALEECIAATPSDEEVACLITDALWYFAQPVADSLGLKRLVLQTGSLFNFHCLVCLPKFLELGLLDPDSKHKDDEPVPGFPHLKVKDIRRAYSHIQESKPILMKMVEETKASSGVIWNSAKELEESELETIQREIPAPSFLLPLPKHYRASSTSLLDTDPSTAQWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDDGFVKDAIVEAINTVMKEEEGAEYRANAQKLKDKLDRSIAPGGSSYESLERLQSYISSL

SEQ ID NO: 326 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEEFREALEECIADTPSDEEVACLITDALWYFAQPVADSLNLKRLVLQTGSLFNFHCLVCLPKFLELGLLDPDSKHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSEKLKVGVYLDDGFVKEKIVEAIKTVMVEEEGAEYRANAQKLKDKLDRSIAPGGSSYESLERLQSYISSL

SEQ ID NO: 327 - protein, 457 aa, synthetic construct
MPNTNPTTVRRRRVVMFPVPFPGHLNPMLQLANVLYRRGFEITILHTNFNAPKTSLYPHFQFRQILDNDPQPEWLRNLPTTGPGVGARIPVINKHGAEELREALLECIADTPSDEEVACLITDALWYFAQPVADSLGLKRLVLQTGSLFNFHCLVCLPKFLELGLLDPDSTHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLDPEDFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKVGVYLDNGFVKDKIVEAINTVMKEEEGAEYRANAQKLKDKLDRSIAPGGSSYESLERLQSYISSL

SEQ ID NO: 328 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEELREALEECIAATPSDEEVACLITDALWYFAQPVADELGLKRLVLQTGSLFNFHCLVCLPKFLELGLLDPDSTHKDDEPVPGFPHLKVKDIRRAYSHIQESKPILMKMVEETKASSGVIWNSAKELEESELETIQREIPAPSFLLPLPKHYRASSTSLLDTDPSTAAWLDTQPAGSVLYVGFGSQSSLEPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDDGFVPEKIVEAINTVMVEEEGAEYRANAQKLKDKLDRSIAPGGSSYESLERLQSYISSL

SEQ ID NO: 329 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEEFREALEECIADTPSDEEVACLITDALWYFAQPVADELNLKRLVLQTGSLFNFHCLVCLPKFLELGLLDPDSKHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTDKLKIGVYLDNGFVPDKIVEAINTVMVEEEGAEYRANAQALKDKLDRSIAPGGSSYESLERLQSYISSL

SEQ ID NO: 330 - protein, 457 aa, synthetic construct
MPNTNPTTVRRRRVVMFPVPFPGHLNPMLQLANVLYRRGFEITILYTNFNAPKTSLYPHFQFRQILDNDPQPEWLRNLPTTGPGVGARIPVINKHGAEEFREALEECIADTPSDEEVACLITDALWYFAQPVADSLGLKRLVLQTGSLFNFHCLVCLPKFLELGLLDPDSKHKDDEPVPGFPHLKVKDIRRAYSHIQESKPILMKMVEETKASSGVIWNSAKELEESELETIQREIPAPSFLLPLPKHYRASSTSLLDTDPSTAQWLDTQPPGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSEKLKVGVYLDDGFVPDKIVEAINTVMKEEEGEVYRANAQKLKDKLDRSIAPGGSSYESLERLQSYISSL

SEQ ID NO: 331 - protein, 457 aa, synthetic construct
MPNTNPTTVRRRRVVMFPVPFPGHLNPMLQLANVLYRRGFEITILYTNFNAPKTSLYPHFQFVPILDNDPQPEWLRNLPTTGPGVGARIPVINKHGAEQLREALEACIAATPSDEEVACLITDALWYFAQPVADSLGLKRLVLQTGSLFNFHCLVCLPKFLELGLLDPDSTHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSEKLKVGVYLDDGFVPDKIVEAINTVMKEEEGKEYRANAQKLKDKLDRSIAPGGSSYESLERLQSYISSL

SEQ ID NO: 332 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEQFREALEECIADTPSDEEVACLITDALWYFAQPVADELGLKRLVLQTGSLFNFHCLVCLPKFLELGLLDPDSTHKDDEPVPGFPHLKVKDIRRAYSHIQESKPILMKMVEETRASSGVIWNSAKELEESELETIQREIPAPSFLLPLPKHYRASSTSLLDTDPSTAAWLDTQPAGSVLYVGFGSQSSLEPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDDGFVKDAIVEAINTVMKEEEGKEYRANAQALKDKLDRSIAPGGSSYESLERLQSYISSL

SEQ ID NO: 333 - protein, 457 aa, synthetic construct
MPNTNPTTVRRRRVVMFPVPFPGHLNPMLQLANVLYRRGFEITILHTNFNAPKTSLYPHFQFRQILDNDPQPEWLRNLPTTGPGVGARIPVINKHGAEEFREALEECIAATPSDEEVACLITDALWYFAQPVADSLGLKRLVLQTGSLFNFHCLVCLPKFLELGLLDPDSTHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTDKLKIGVYLDNGFVPEKIVEAINTVMKEEEGEVYRANAQKLKDKLDRSIAPGGSSYESLERLQSYISSL

SEQ ID NO: 334 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEEFREALEECIADTPSDEEVACLITDALWYFAQPVADSLNLKRLVLQTGSLFNFHCLVCLPKFLELGLLDPDSKHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLEPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTDKLKVGVYLDNGFVKDAIVEAINTVMKEEEGEVYRANAQKLKDKLDRSIAPGGSSYESLERLQSYISSL

SEQ ID NO: 335 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEEFREALEECIAATPSDEELACLITDALWYFAQPVADELGLKRLVLQTGSLFNFHCLVCLPKFLELGLLDPDSTHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPPGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTDKLKVGVYLDDGFVKDKIVEAINTVMVEEEGAEYRANAKKLKDKLDRSIAPGGSSYESLERLQSYISSL

SEQ ID NO: 336 - protein, 457 aa, synthetic construct
MPNTNPTTVRRRRVVMFPVPFPGHLNPMLQLANVLYRRGFEITILYTNFNAPKTSLYPHFQFVPILDNDPQPEWLRNLPTTGPGVGARIPVINKHGAEQLREALEECIAATPSDEEVACLITDALWYFAQPVADELGLKRLVLQTGSLFNFHCLVCLPKFLELGLLDPDSKLKDDEPVPGFPHLKVKDIRRAYSHIQESKPILMKMVEETRASSGVIWNSAKELEESELETIQREIPAPSFLLPLPKHYRASSTSLLDTDPSTAAWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKVGVYLDDGFVKEAIVEAIKTVMKEEEGAEYRANAQALKDKLDRSIAPGGSSYESLERLQSYISSL

SEQ ID NO: 337 - protein, 457 aa, synthetic construct
MPNTNPTTVRRRRVVMFPVPFPGHLNPMLQLANVLYRRGFEITILHTNFNAPKTSLYPHFQFRQILDNDPQPEWLRNLPTTGPGVGARIPVINKHGAEEFREALEECIAATPSDEEVACLITDALWYFAQPVADELGLKRLVLQTGSLFNFHCLVCLPKFLELGLLDPDSKHKDDEPVPGFPHLKVKDIRRAYSHIQESKPILMKMVEETRASSGVIWNSAKELEESELETIQREIPAPSFLLPLPKHYRASSTSLLDTDPSTAAWLDTQPPGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDNGFVKDAIVEAIKTVMVEEEGEVYRANAQKLKDKLDRSIAPGGSSYESLERLQSYISSL

SEQ ID NO: 338 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEEFREALEECIAATPSDEEVACLITDALWYFAQPVADSLGLKRLVLQTGSLFNFHCLVCLPKFLELGLLDPDSTHKDDEPVPGFPHLKVKDIRRAYSHIQESKPILMKMVEETKASSGVIWNSAKELEESELETIQREIPAPSFLLPLPKHYRASSTSLLDTDPSTAQWLDTQPAGSVLYVGFGSQSSLEPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSEKLKVGVYLDNGFVKEKIVEAINTVMVEEEGEVYRANAKKLKDKLDRSIAPGGSSYESLERLQSYISSL

SEQ ID NO: 339 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEELRAALEECIAATPSDEEVACLITDALWYFAQPVADELGLKRLVLQTGSLFNFHCLVCLPKFLELGLLDPDSTHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPPGSVLYVGFGSQSSLDPEEFLALAEGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSDKLKIGVYLDDGFVKDAIVEAIKTVMKEEEGAEYRANAQKLKDKLDRSIAPGGSSYESLERLQSYISSL

SEQ ID NO: 340 - protein, 457 aa, synthetic construct
MPNTNPTTVRRRRVVMFPVPFPGHLNPMLQLANVLYRRGFEITILYTNFNAPKTSLYPHFQFVPILDNDPQPEWLRNLPTTGPGVGARIPVINKHGAEELREALEECIAATPSDEEVACLITDALWYFAQPVADSLNLKRLVLQTGSLFNFHCLVCLPKFLELGLLDPDSTHKDDEPVPGFPHLRVKDIRRAYSHIQESKPILMKMVEETRASSGVIWNSAKELEESELETIQREIPAPSFLLPLPKHYRASSTSLLDTDPSTAAWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDNGFVKDKIVEAINTVMKEEEGAEYRANAQALKDKLDRSIAPGGSSYESLERLQSYISSL

SEQ ID NO: 341 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEQLRAALEECIADTPSDEEVACLITDALWYFAQPVADSLGLKRLVLQTGSLFNFHCLVCLPKFLELGLLDPDSKHKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLDPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDNGFVKEAIVEAIKTVMVEEEGAEYRANAQALKDKLDRSIAPGGSSYESLERLQSYISSL

SEQ ID NO: 342 - protein, 457 aa, synthetic construct
MPNTNPTTVRRRRVVMFPVPFPGHLNPMLQLANVLYRRGFEITILYTNFNAPKTSLYPHFQFRPILDNDPQPEWLRNLPTTGPGVGARIPVINKHGAEEFRAALEECIADTPSDEEVACLITDALWYFAQPVADSLNLKRLVLQTGSLFNFHCLVCLPKFLELGLLDPDSKHKDDEPVPGFPHLKVKDIRRAYSHIQESKPILMKMVEETKASSGVIWNSAKELEESELETIQREIPAPSFLLPLPKHYRASSTSLLDTDPSTAAWLDTQPPGSVLYVGFGSQSSLEPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSEKLKIGVYLDNGFVKDKIVEAINTVMKEEEGAEYRANAQKLKDKLDRSIAPGGSSYESLERLQSYISSL

SEQ ID NO: 343 - protein, 457 aa, synthetic construct
MPNTNPTTVRRRRVVMFPVPFPGHLNPMLQLANVLYRRGFEITILHTNFNAPKTSLYPHFQFRPILDNDPQPEWLRNLPTTGPGVGARIPVINKHGAEELREALEECIAATPSDEEVACLITDALWYFAQPVADSLNLKRLVLQTGSLFNFHCLVCLPKFLELGLLDPDSTLKDDEPVPGFPHLKVKDIRRAYSHIQESKPILMKMVEETKASSGVIWNSAKELEESELETIQREIPAPSFLLPLPKHYRASSTSLLDTDPSTAQWLDTQPAGSVLYVGFGSQSSLDPEDFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMSEKLKVGVYLDDGFVKDKIVEAINTVMVEEEGAEYRANAQALKDKLDRSIAPGGSSYESLERLQSYISSL

SEQ ID NO: 344 - protein, 457 aa, synthetic construct
MPNTNPTTVRRRRVVMFPVPFPGHLNPMLQLANVLYRRGFEITILYTNFNAPKTSLYPHFQFVPILDNDPQPEWLRNLPTTGPGVGARIPVINKHGAEQFRAALEECIAATPSDEEVACLITDALWYFAQPVADSLGLKRLVLQTGSLFNFHCLVCLPKFLELGLLDPDSTLKDDEPVPGFPHLKVKDIRRAYSHIQESKPILMKMVEETRASSGVIWNSAKELEESELETIQREIPAPSFLLPLPKHYRASSTSLLDTDPSTAQWLDTQPAGSVLYVGFGSQSSLEPEEFLALADGLVASGQPFLWLVRPGFVKGYEWIEFLPDGYLGEKGRIVKSAPQQEVLAHPAIGAFWTHGGWNGTMEAVCEGVPMIFSDFGLDQPLNARYMTEKLKIGVYLDDGFVKDKIVEAINTVMVEEEGAEYRANAQKLKDKLDRSIAPGGSSYESLERLQSYISSL

SEQ ID NO: 345 - protein, 457 aa, synthetic construct
MPNTNTTTRRRRRIIMFPVPFPGHLNPMLQLANVLYRRGFEITILYTNFNKPKTSNYPHFQFRPILDNDPQPEWLRNLPTTGPGVGARIPVINKHGADELKEALEALIAETESDEEVACLITDALWYFAQPVADSLGLRRLVLQTGSLFNFHCLVSLPRFLELGLLDPDDKHKPDEPVPGFPMLKVADIKRAYSHIQESKPILEKMVKETRASSGVIWNSAKELEESELETIWREIPAPSFLLPLPKHYRASSTSLLDTDRSTFQWLDTQPAGSVLYVGFGSQSELDPEEFLALADGLVLSGQPFLWLVRPGFVKGYEWIEALPDGYLGEKGRIVKSAPQQEVLAHGAIGAFWTHGGWNGTMESVCEGVPMIFSDFGLDQPLNARYMSEKLKVGVYLDDGFVKDAIAEAINLVMVEEKGKPYRANAQKLKDKLDRSIAPGGSSYESLESLQSYISSL

SEQ ID NO: 346 - protein, 457 aa, synthetic construct
MPNTNTTTRRRRRIIMFPVPFPGHLNPMLQLANVLYRRGFEITILYTNFNKPKTSNYPHFQFRPILDNDPQPEWLRNLPTTGPGVGARIPVINKHGADEFRAALEALIAETESDEEVACLITDALWYFAQPVADSLNLRRLVLQTGSLFNFHCLVSLPRFLELGLLDPNDPHRDEEPVPGFPHLKVADIKRAYSHIQESKPILEKMVKETKASSGVIWNSAKELEESELETIWREIPAPSFLLPLPKHYRASSTSLLDTDRSTFQWLDTQPPGSVLYVGFGSQSELDPEEFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMTEKLKVGVYLDDGFVKEKIVEAINLVMKEERGEPYRQNAQVLKDKLDRSIAPGGSSYESLESLQSYISSL

SEQ ID NO: 347 - protein, 457 aa, synthetic construct
MPNTNPTTVRRRRVVLFPVPFPGHLNPMLQLANVLYRRGFEITILYTNFNAPKTSLYPHFQFRPILDNDPQPEWLRNLPTTGPGVGARIPVINKHGAEELREALLELIADTESDEEVACLITDALWYFAQPVADSLNLRRLVLQTGSLFNFHCLVSLPRFLELGLLDPDDTTRDDEPVPGFPHLKVADIKRAYSHIQESKPILEKMVKETRASSGVIWNSAKELEESELETIWREIPAPSFLLPLPKHYRASSTSLLDTDRSTFQWLDTQPPGSVLYVGFGSQSSLDPEEFLALADGLVLSGQPFLWLVRPGFVKGYEWIEALPDGYLGERGRIVKSAPQQEVLAHPAIGAFWTHGGWNGTMEAVCEGVPMIFSDFGLDQPLNARYMSEKLKVGVYLDDGFVPDKIVEAINKVMKEEKGEPYRANAKALKDKLDRSIAPGGSSYESLERLQSYISSL

SEQ ID NO: 348 - protein, 457 aa, synthetic construct
MPNTNPTTRRRRRIVMFPVPFPGHLNPMLQLANVLYRRGFEITILYTNFNKPKTSNYPHFQFVPILDNDPQPEWLRNLPTTGPGVGARIPVINKHGAEEFKEALLKLIADTESDEEVACLITDALWYFAQPVADSLGLRRLVLQTGSLFNFHCLVCLPRFLELGLLDPDDTTKDDEPAPGFPHLRVADIRRAYSHIQESKPILEKMVKQTKASSGVIWNSAKELEESELETIWREIPAPSFLLPLPKHYRASSTSLLDTDRSTFQWLDTQPARSVLYVGFGSQSSLDPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMTDKLKIGVYLDDGFVKDKIAEAINLVMEEERGEPYRANAQVLKDKLDRSIAPGGSSYESLERLQSYISSL

SEQ ID NO: 349 - protein, 457 aa, synthetic construct
MPNTNPTTRRRRRIILFPVPFPGHLNPMLQLANVLYRRGFEITILYTNFNAPKTSNYPHFQFVPILDNDPQPEWLRNLPTTGPGVGARIPVINKHGADELREALEALIAETESDEELACLITDALWYFAQPVADELGLRRLVLQTGSLFNFHCLVSLPEFLELGLLDPDDKHKDEEPVPGFPHLKVADIKRAYSHIQESKPILEKMVKETRASSGVIWNSAKELEESELETIWREIPAPSFLLPLPKHYRASSTSLLDTDRSTFQWLDTQPPGSVLYVGFGSQSSLDPEEFLALADGLVLSGQPFLWLVRPGFVKGYEWIEALPDGYLGEKGRIVKSAPQQEVLAHPAIGAFWTHGGWNGTMESVCEGVPMIFSDFGLDQPLNARYMSDKLKIGVYLDDGFVKDKIAEAINLVMEEERGEPYRANAKALKDKLDRSIAPGGSSYESLERLQSYISSL

SEQ ID NO: 350 - protein, 457 aa, synthetic construct
MPNTNTTTVRRRRVILFPVPFPGHLNPMLQLANVLYRRGFEITILYTNFNKPKTSNYPHFQFRPILDNDPQPEWLRNLPTTGPGVGARIPVINKHGAEQFRAALEECIAETESDEEVACLITDALWYFAQPVADSLNLRRLVLQTGSLFNFHCLVCLPRFLELGLLDPNDPHKPEEPVPGFPHLKVADIKRAYSHIQESKPILEKMVKETKASSGVIWNSFKELEESELETIWREIPAPSFLLPLPKHYRASSTSLLDTDRSTFQWLDTQPAGSVLYVGFGSQSSLDPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKIGVYLDNGWVKEAIAEAINLVMVEEKGEPYRQNAQVLKDKLDRSIAPGGSSYESLERLQSYISSL

SEQ ID NO: 351 - protein, 457 aa, synthetic construct
MPNTNPTTRRRRRVILFPVPFPGHLNPMLQLANVLYRRGFEITILYTNFNKPKTSNYPHFQFRQILDNDPQPEWLRNLPTTGPGVGARIPVINKHGADQFREALEACIAETESDEEVACLITDALWYFAQPVADSLNLRRLVLQTGSLFNFHCLVSLPRFLELGLLDPDDTHKDDEPVPGFPHLKVADIKRAYSHIQESKPILEKMVKETRASSGVIWNSAKELEESELETIWREIPAPSFLLPLPKHYRASSTSLLDTDPSTFQWLDTQPAGSVLYVGFGSQSSLDPEDFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMTEKLKVGVYLDNGFVKDKIVEAINLVMKEEKGKPYRQNARKLKDKLDRSIAPGGSSYESLERLQSYISSL

SEQ ID NO: 352 - protein, 457 aa, synthetic construct
MPNTNTTTRRRRRIVMFPVPFPGHLNPMLQLANVLYRRGFEITILYTNFNKPKTSNYPHFQFRQILDNDPQPEWLRNLPTTGPGVGARIPVINKHGAERFKAALEECIAETESDEEVACLITDALWYFAQPVADSLNLRRLVLQTGSLFNFHCLVSLPRFLELGLLDPDSTHKDDEPVPGFPHLKVADIRRAYSHIQESKPILEKMVKETRKSSGVIWNSAKELEESELETIWREIPAPSFLIPLPKHYRASSTSLLDTDRSTFAWLDTQPAESVLYVGFGSQSSLDPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMTEKLKIGVYLDNGWVKEAIAEAIHLVMVEEKGEPYRANAQKLKDKLDRSIAPGGSSYESLERLQSYISSL

SEQ ID NO: 353 - protein, 457 aa, synthetic construct
MPNTNPTTRRRRRVILFPVPFPGHLNPMLQLANVLYSRGFEITILHTNFNAPKTSNYPHFQFRPILDNDPQPEWLRNLPTHGPGVGARIPVINKHGAEEFRAALEQLIAETESDEEVACLITDALWYFAQPVADSLGLRRLVLQTGSLFNFHCLVSLPRFLELGLLDPDDPHRDDEPVPGFPHLKVADIKRAYSHIQESKPILEKMVKETRASSGVIWNSAKELEESELETIWREIPAPSFLLPLPKHYRASSTSLLDTDRSTFAWLDQQPPGSVLYVGFGSQSELDPEEFLALADGLVLSGQPFLWLVRPGFVKGYEWIEALPDGYLGPKGRIVKSAPQQEVLAHGAIGAFWTHGGWNGTMESVCEGVPMIFSDFGLDQPLNARYMTEKLKIGVYLDNGWVKDAIAEAIKEVMVEEKGEPYRQNAQVLKDKLDRSIAPGGSSYESLESLQSYISSL

SEQ ID NO: 354 - protein, 457 aa, synthetic construct
MPNTNTTTRRRRRVILFPVPFPGHLNPMLQLANVLYSRGFEITILYTNFNKPKTSNYPHFQFRQILDNDPQPEWLRNLPTTGPGVGARIPVINKHGADEFREALEECIAATESDEEVACLITDALWYFAQPVADSLNLRRLVLQTGSLFNFHCLVCLPEFLELGLLDPDDKHKDEEPVPGFPHLKVADIKRAYSHIQESKPILEKMVKETRASSGVIWNSAKELEESELETIWREIPAPSFLLPLPKHYRASSTSLLDTDRSTFQWLDTQPAGSVLYVGFGSQSSLEPAEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMTEKLKVGVYLDDGFVPEKIAEAINLVMVEERGEPYRANAQKLKQKLDRSIAPGGSSYESLERLQSYISSL

SEQ ID NO: 355 - protein, 457 aa, synthetic construct
MPNTNTTTRRRRRIILFPVPFPGHLNPMLQLANVLYRRGFEITILYTNFNAPKTSNYPHFQFRQILDNDPQPEWLRNLPTTGPGVGARIPVINKHGAEEFREALEALIAETESDEEVACLITDALWYFAQPVADSLNLRRLVLQTGSLFNFHCLVSLPKFLELGLLDPNDKHKDDEPVPGFPHLKVADIKRAYSHIQESKPILEKMVKETRASSGVIWNSFKELEESELETIWREIPAPSFLLPLPKHYRASSTSLLDTDRSTAQWLDTQPARSVLYVGFGSQSSLDPEEFLAIADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKVGVYLDDGFVKEKIVEAINKVMEEEEGEPYRQNAQKLKDKLDRSIAPGGSSYESLERLQSYISSL

SEQ ID NO: 356 - protein, 457 aa, synthetic construct
MPNTNPTTRRRRRIILFPVPFPGHLNPMLQLANVLYRRGFEITILYTNFNKPKTSNYPHFQFRPILDNDPQPEWLRNLPTTGPGVGARIPVINKHGADELEAALEECIAETESDEEVACLITDALWYFAQPVADELGLRRLVLQTGSLFNFHCLVSLPRFLELGLLDPNDKHKPDEPVPGFPHLKVADIRRAYSHIQESKPILEKMVEETKASSGVIWNSAKELEESELETIWREIPAPSFLLPLPKHYRASSTSLLDTDRSTFQWLDTQPAGSVLYVGFGSQSSLEPEEFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMTEKLKIGVYLDDGFVKEKIAEAINLVMKEERGEPYRANAKKLKQKLDRSIAPGGSSYESLESLQSYISSL

SEQ ID NO: 357 - protein, 457 aa, synthetic construct
MPNTNTTTRRRRRIILFPVPFPGHLNPMLQLANVLYRRGFEITILYTNFNKPKTSNYPHFQFRTILDNDPQPEWLRNLPTTGPGVGARIPVINKHGADELREALEALIAETESDEEVACLITDALWYFAQPVADSLNLRRLVLQTGSLFNFHCLVCLPRFLELGLLDPDDTHKDDEPVPGFPHLKVADIKRAYSHIQESKPILEKMVKETRASSGVIWNSAKELEESELETIWREIPAPSFLIPLPKHYRASSTSLLDTDRSTFQWLDTQPAGSVLYVGFGSQSSLDPEDFLALADGLVLSGQPFLWLVRPGFVKGYEWIEALPDGYLGPRGRIVKSAPQQEVLAHPAIGAFWTHGGWNGTMEAVCEGVPMIFSDFGLDQPLNARYMSDKLKIGVYLDNGFVKEAIAEAINEVMVEERGEPYRANAKALKQKLDRSIAPGGSSYESLESLQSYISSL

SEQ ID NO: 358 - protein, 457 aa, synthetic construct
MPNTNTTTRRRRRVIMFPVPFPGHLNPMLQLANVLYRRGFEITILYTNFNKPKTSNYPHFQFRPILDNDPQPEWLRNLPTTGPGVGARIPVINKHGADEFKEALEALIAETESDEEVACLITDALWYFAQPVADSLNLRRLVLQTGSLFNFHCLVSLPRFLELGLLDPNDPHRPDEPVPGFPHLKVADIRRAYSHIQESKPILEKMVKETRASSGVIWNSFKELEESELETVWREIPAPSFLLPLPKHYRASSTSLLDTDPSTFQWLDTQPPGSVLYVGFGSQSSLDPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSDKLKIGVYLDDGFVKDKIAEAIKLVMVEEKGEPYRANAKVLKDKLDRSIAPGGSSYESLERLQSYISSL

SEQ ID NO: 359 - protein, 457 aa, synthetic construct
MPNTNTTTRRRRRIVLFPVPFPGHLNPMLQLANVLYRRGFEITILYTNFNKPKTSNYPHFQFVPILDNDPQPEWLRNLPTTGPGVGARIPVINKHGAEELREALEACIAETESDEEVACLITDALWYFAQPVADSLNLRRLVLQTGSLFNFHCLVSLPRFLELGLLDPNSKTKDDEPVPGFPHLKVADIRRAYSHIQESKPILEKMVEETKASSGVIWNSAKELEESELETIWREIPAPSFLLPLPKHYRASSTSLLDTDRSTFQWLDQQPAESVLYVGFGSQSELDPEDFLAIADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMSEKLKVGVYLDDGFVKEKIVEAINLVMKEEKGEVYRANAQALKDKLDRSIAPGGSSYESLERLQSYISSL

SEQ ID NO: 360 - protein, 457 aa, synthetic construct
MPNTNTTTRRRRRIVLFPVPFPGHLNPMLQLANVLYRRGFEITILYTNFNKPKTSNYPHFQFVPILDNDPQPEWLRNLPTTGPGVGARIPVINKHGADEFKAALLALIAETESDEEVACLITDALWYFAQPVADELNLRRLVLQTGSLFNFHCLVSLPRFLELGLLDPNDTHKPEEPVPGFPHLKVADIRRAYSHIQESKPILEKMVKETKASSGVIWNSAKELEESELETIWREIPAPSFLLPLPKHYRASSTSLLDTDRSTFAWLDTQPAGSVLYVGFGSQSSLDPEEFLALADGLVLSGQPFLWLVRPGFVKGYEWIEALPDGYLGPQGRIVKSAPQQEVLAHPAIGAFWTHGGWNGTMEAVCEGVPMIFSDFGLDQPLNARYMTEKLKIGVYLDDGFVPDAIVEAINLVMKAEKGEPYRANARALKQKLDRSIAPGGSSYESLERLQSYISSL

SEQ ID NO: 361 - protein, 457 aa, synthetic construct
MPNTNTTTRRRRRVVLFPVPFPGHLNPMLQLANVLYRRGFEITILYTNFNKPKTSNYPHFQFRTILDNDPQPEWLRNLPTTGPGVGARIPVINKHGADEFKEALEELIAETESDEEVACLITDALWYFAQPVADSLGLRRLVLQTGSLFNFHCLVSLPRFLELGLLDPDDKHKDDEPVPGFPHLKVADIKRAYSHIQESKPILEKMVKETRASSGVIWNSAKELEESELETIWREIPAPSFLLPLPKHYRASSTSLLDTDRSTFAWLDQQPPGSVLYVGFGSQSSLDPEEFLALADGLVLSGQPFLWLVRPGFVKGYEWIEALPDGYLGEQGRIVKSAPQQEVLAHPAIGAFWTHGGWNGTMEAVCEGVPMIFSDFGLDQPLNARYMTEKLKVGVYLDNGWVKEAIAEAINEVMEEEKGAPYRQNARKLKDKLDRSIAPGGSSYESLERLQSYISSL

SEQ ID NO: 362 - protein, 457 aa, synthetic construct
MPNTNTTTRRRRRIILFPVPFPGHLNPMLQLANVLYSRGFEITILYTNFNKPKTSLYPHFQFRTILDNDPQPEWLRNLPTTGPGVGARIPVINKHGADEFREALEALIAETESDEEVACLITDALWYFAQPVADSLNLRRLVLQTGSLFNFHCLVSLPRFLELGLLDPDDPTKDEEPVPGFPHLKVADIKRAYSHIQESKPILEKMVKETRASSGVIWNSAKELEESELETIWREIPAPSFLLPLPKHYRASSTSLLDTDRSTFAWLDTQPPESVLYVGFGSQSSLEPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDNGFVKEKIAEAINLVMVEEKGEPMRQNAKALKDKLDRSIAKGGSSYESLESLQSYISSL

SEQ ID NO: 363 - protein, 457 aa, synthetic construct
MPNTNTTTRRRRRVVLFPVPFPGHLNPMLQLANVLYRRGFEITILHTNFNAPKTSNYPHFQFRQILDNDPQPEWLRNLPTTGPGVGARIPVINKHGADEFRAALEQCIAETESDEEVACLITDALWYFAQPVADSLNLRRLVLQTGSLFNFHCLVCLPRFLELGLLDPNDTHKDDEPVPGFPHLKVADIKRAYSHIQESKPILEKMVKETRASSGVIWNSAKELEESELETIWREIPAPSFLLPLPKHYRASSTSLLDTDRSTFAWLDTQPPRSVLYVGFGSQSSLDPEEFLAIADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKIGVYLDDGFVKDKIVEAIRLVMKEEEGEPMRANAKKLKDKLDRSIAPGGSSYESLERLQSYISSL

SEQ ID NO: 364 - protein, 457 aa, synthetic construct
MPNTNTTTRRRRRIVMFPVPFPGHLNPMLQLANVLYRRGFEITILYTNFNKPKTSNYPHFQFVPILDNDPQPEWLRNLPTTGPGVGARIPVINKHGAEELKAALLQLIADTESDEEVACLITDALWYFAQPVADSLNLRRLVLQTGSLFNFHCLVSLPRFLELGLLDPNDKHKDDEPVPGFPHLRVADIKRAYSHIQESKPILEKMVKETKASSGVIWNSAKELEESELETIWREIPAPSFLLPLPKHYRASSTSLLDTDRSTAQWLDTQPAGSVLYVGFGSQSSLEPEEFLALADGLVLSGQPFLWLVRPGFVKGYEWIEALPDGYLGPKGRIVKSAPQQEVLAHPAIGAFWTHGGWNGTMESVCEGVPMIFSDFGLDQPLNARYMSEKLKIGVYLDDGFVKDKIANAINLVMKEEKGAPYRANARALKDKLDRSIAPGGSSYESLERLQSYISSL

SEQ ID NO: 365 - protein, 457 aa, synthetic construct
MPNTNTTTRRRRRIILFPVPFPGHLNPMLQLANVLYRRGFEITILHTNFNKPKTSNYPHFQFRPILDNDPQPEWLRNLPTHGPGVGARIPVINKHGADEFEEALEALIAETESDEEVACLITDALWYFAQPVADELNLRRLVLQTGSLFNFHCLVSLPRFLELGLLDPDDKHKDDEPVPGFPHLRVADIKRAYSHIQESKPILEKMVKETRASSGVIWNSAKELEESELETIWREIPAPSFLLPLPKHYRASSTSLLDTDRSTFQWLDQQPPGSVLYVGFGSQSSLDPEEFLALADGLVLSGQPFLWLVRPGFVKGYEWIEALPDGYLGPQGRIVKSAPQQEVLAHGAIGAFWTHGGWNGTMEAVCEGVPMIFSDFGLDQPLNARYMSDKLKVGVYLDDGFVKDKIAEAIHLVMEEEKGEVYRQNAKKLKDKLDRSIAPGGSSYESLESLQSYISSL

SEQ ID NO: 366 - protein, 457 aa, synthetic construct
MPNTNTTTRRRRRVILFPVPFPGHLNPMLQLANVLYRRGFEITILYTNFNKPKTSNYPHFQFVPILDNDPQPEWLRNLPTTGPGVGARIPVINKHGADEFRAALLQCIADTESDEEVACLITDALWYFAQPVADSLNLRRLVLQTGSLFNFHCLVSLPRFLELGLLDPNDPHKDDEPVPGFPHLKVADIKRAYSHIQESKPILEKMVKETKASSGVIWNSFKELEESELETIWREIPAPSFLLPLPKHYRASSTSLLDTDRSTFQWLDTQPAESVLYVGFGSQSELDPEEFLALADGLVLSGQPFLWLVRPGFVKGYEWIEALPDGYLGEQGRIVKSAPQQEVLAHGAIGAFWTHGGWNGTMEAVCEGVPMIFSDFGLDQPLNARYMTDKLKVGVYLDNGFVKDKIAEAINLVMKEERGEPYRANAQKLKQKLDRSIAPGGSSYESLERLQSYISSL

SEQ ID NO: 367 - protein, 457 aa, synthetic construct
MPNTNTTTRRRRRVVLFPVPFPGHLNPMLQLANVLYSRGFEITILYTNFNKPKTSLYPHFQFRQILDNDPQPEWLRNLPTTGPGVGARIPVINKHGAEELREALLACIAETESDEEVACLITDALWYFAQPVADSLNLRRLVLQTGSLFNFHCLVSLPRFLELGLLDPDDKTKDDEPVPGFPHLKVADIRRAYSHIQESKPILEKMVKETKASSGVIWNSAKELEESELETIWREIPAPSFLLPLPKHYRASSTSLLDTDPSTFQWLDQQPPGSVLYVGFGSQSELDPEDFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDNGFVKDAIAAAINEVMVEEEGKPYRANAQKLKDKLDRSIAPGGSSYESLESLQSYISSL

SEQ ID NO: 368 - protein, 457 aa, synthetic construct
MPNTNTTTRRRRRVILFPVPFPGHLNPMLQLANVLYRRGFEITILYTNFNKPKTSNYPHFQFRPILDNDPQPEWLRNLPTTGPGVGARIPVINKHGAEELKEALLECIAETESDEEVACLITDALWYFAQPVADSLGLRRLVLQTGSLFNFHCLVCLPRFLELGLLDPNDPHKDDEPVPGFPILRVADIRRAYSHIQESKPILEKMVKETKASSGVIWNSAKELEESELETIWREIPAPSFLLPLPKHYRASSTSLLDTDRSTFAWLDTQPARSVLYVGFGSQSSLEPEEFLALADGLVLSGQPFLWLVRPGFVKGYEWIEALPDGYLGERGRIVKSAPQQEVLAHPAIGAFWTHGGWNGTMEAVCEGVPMIFSDFGLDQPLNARYMTEKLKIGVYLDDGFVKDKIAEAINLVMEEEKGEPYRANARALKDKLDRSIAKGGSSYESLERLQSYISSL

SEQ ID NO: 369 - protein, 457 aa, synthetic construct
MPNTNPTTRRRRRIVLFPVPFPGHLNPMLQLANVLYSRGFEITILYTNFNKPKTSNYPHFQFRPILDNDPQPEWLRNLPTHGPGVGARIPVINKHGADEFREALEALIAETESDEEVACLITDALWYFAQPVADSLGLRRLVLQTGSLFNFHCLVSLPRFLELGLLDPNDPTKPEEPVPGFPHLKVADIKRAYSHIQESKPILEKMVKETKASSGVIWNSAKELEESELETIWREIPAPSFLLPLPKHYRASSTSLLDTDRSTFQWLDQQPAGSVLYVGFGSQSELDPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMTDKLKVGVYLDNGFVKDKIAEAINLVMKEEKGEVYRANAQALKDKLDRSIAPGGSSYESLERLQSYISSL

SEQ ID NO: 370 - protein, 457 aa, synthetic construct
MPNTNPTTRRRRRVILFPVPFPGHLNPMLQLANVLYRRGFEITILYTNFNKPKTSLYPHFQFRPILDNDPQPEWLRNLPTTGPGVGARIPVINKHGADEFKEALEALIAETESDEEVACLITDALWYFAQPVADSLGLRRLVLQTGSLFNFHCLVSLPRFLELGLLDPDDPHKPDEPVPGFPHLKVADIKRAYSHIQESKPILEKMVKETKASSGVIWNSAKELEESELETIWREIPAPSFLLPLPKHYRASSTSLLDTDRSTFQWLDTQPPGSVLYVGFGSQSSLEPAEFLALADGLVLSGQPFLWLVRPGFVKGHEWIEALPDGYLGEKGRIVKSAPQQEVLAHPAIGAFWTHGGWNGTMEAVCEGVPMIFSDFGLDQPLNARYMSEKLKVGVYLDNGFIKDAIVEAIKLVMEEEKGEPYRANARALKDKLDRSIAPGGSSYESLESLQSYISSL

SEQ ID NO: 371 - protein, 457 aa, synthetic construct
MPNTNPTTRRRRRVILFPVPFPGHLNPMLQLANVLYRRGFEITILYTNFNAPKTSLYPHFQFRPILDNDPQPEWLRNLPTTGPGVGARIPVINKHGAEEFKEALEALIADTPSDEEVACLITDALWYFAQEVADSLNLRRLVLQTGSLFNFHCLVSLPRFLELGLLDPDSTHRDDEPVPGFPHLKVADIKRAYSHIQESKPILEKMVEETRASSGVIWNSAKELEESELETIWREIPAPSFLLPLPKHYRASSTSLLDTDRSTAQWLDTQPAGSVLYVGFGSQSSLDPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKVGVYLDDGFVKEKIVEAINLVMVAEKGKPYRQNAQALKDKLDRSIAPGGSSYESLERLQSYISSL

SEQ ID NO: 372 - protein, 457 aa, synthetic construct
MPNTNTTTRRRRRVVLFPVPFPGHLNPMLQLANVLYRRGFEITILYTNFNKPKTSNYPHFQFRPILDNDPQPEWLRNLPTTGPGVGARIPVINKHGAEELREALLALIAETESDEEVACLITDALWYFAQEVADSLGLKRLVLQTGSLFNFHCLVSLPRFLELGLLDPDDPHKDDEPVPGFPHLKVADIKRAYSHIQESKPILEKMVKETKASSGVIWNSFKELEESELETIWREIPAPSFLLPLPKHYRASSTSLLDTDRSTFAWLDTQPARSVLYVGFGSQSSLDPEEFLALADGLVLSGQPFLWLVRPGFVKGHEWIEALPDGYLGPKGRIVKSAPQQEVLAHPAIGAFWTHGGWNGTMESVCEGVPMIFSDFGLDQPLNARYMTEKLKIGVYLDDGWVKDDIVEAINLVMEEEKGAPYRANAQALKQKLDRSIAPGGSSYESLERLQSYISSL

SEQ ID NO: 373 - protein, 457 aa, synthetic construct
MPNTNTTTRRRRRVILFPVPFPGHLNPMLQLANVLYRRGFEITILYTNFNKPKTSNYPHFTFRPILDNDPQPEWLRNLPTHGPGVGARIPVINKHGADEFEAALEALIAETESDEEVACLITDALWYFAQPVADSLNLRRLVLQTGSLFNFHCLVSLPRFLELGLLDPDDPHKDEEPVPGFPHLKVADIKRAYSHIQESKPILEKMVEETRASSGVIWNSAKELEESELETIWREIPAPSFLLPLPKHYRASSTSLLDTDRSTFAWLDQQPPGSVLYVGFGSQSSLEPEEFLALADGLVLSGQPFLWLVRPGFVKGYEWIEALPDGYLGEKGRIVKSAPQQEVLAHPAIGAFWTHGGWNGTMESVCEGVPMIFSDFGLDQPLNARYMSEKLKIGVYLDNGWVPDKIVEAINEVMKEERGAPYRQNAQKLKDKLDRSIAPGGSSYESLERLQSYISSL

SEQ ID NO: 374 - protein, 457 aa, synthetic construct
MPNTNTTTRRRRRVVLFPVPFPGHLNPMLQLANVLYSRGFEITILYTNFNKPKTSNYPHFQFRPILDNDPQPEWLRNLPTTGPGVGARIPVINKHGADEFKEALEALIAETESDEEVACLITDALWYFAQPVADSLNLRRLVLQTGSLFNFHCLVSLPKFLELGLLDPDDTHRDEEPVPGFPMLKVKDIRRAYSHIQESKPILEKMVKETRASSGVIWNSAKELEESELETIWREIPAPSFLLPLPKHYRASSTSLLDTDRSTFAWLDQQPPGSVLYVGFGSQSSLDPAEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMTEKLKVGVYLDDGFVKDKIAEAINLVMVEEKGEPYRQNAQALKDKLDRSIAPGGSSYESLERLQSYISSL

SEQ ID NO: 375 - protein, 457 aa, synthetic construct
MPNTNPTTRRRRRVVMFPVPFPGHLNPMLQLANVLYRRGFEITILHTNFNKPKTSLYPHFQFVPILDNDPQPEWLRNLPTTGPGVGARIPVINKHGAEEFKEALEALIAETESDEEVACLITDALWYFAQPVADSLNLRRLVLQTGSLFNFHCLVSLPRFLELGLLDPNDTHKDEEPVPGFPHLKVADIKRAYSHIQESKPILEKMVKETRASSGVIWNSAKELEESELETIWREIPAPSFLLPLPKHYRASSTSLLDTDRSTFAWLDTQPPGSVLYVGFGSQSSLEPEEFLAIADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKVGVYLDDGFVKERIVAAINLVMKDEKGKEYRANAKALKDKLDRSIAPGGSSYESLERLQSYISSL

SEQ ID NO: 376 - protein, 457 aa, synthetic construct
MPNTNPTTRRRRRIVMFPVPFPGHLNPMLQLANVLYRRGFEITILYTNFNKPKTSNYPHFQFRPILDNDPQPEWLRNLPTTGPGVGARIPVINKHGAEQLKEALEACIAETESDEEVACLITDALWYFAQPVADSLNLRRLVLQTGSLFNFHCLVSLPRFLELGLLDPDDPHKPEEPVPGFPHLKVADIRRAYSHIQESKPILEKMVEETKASSGVIWNSAKELEESELETIWREIPAPSFLLPLPKHYRASSTSLLDTDRSTAAWLDTQPPGSVLYVGFGSQSSLDPEDFLALADGLVLSGQPFLWLVRPGFVKGYEWIEALPDGYLGPQGRIVKSAPQQEVLAHGAIGAFWTHGGWNGTMEAVCEGVPMIFSDFGLDQPLNARYMSEKLKVGVYLDDGFVKDKIAEAINLVMEEERGEPYRANAKKLKQKLDRSIAPGGSSYESLESLQSYISSL

SEQ ID NO: 377 - protein, 457 aa, synthetic construct
MPNTNTTTRRRRRVILFPVPFPGHLNPMLQLANVLYRRGFEITILYTNFNKPKTSNYPHFQFRPILDNDPQPEWLRNLPTTGPGVGARIPVINKHGADEFREALEALIAETESDEEVACLITDALWYFAQPVADSLGLRRLVLQTGSLFNFHCLVSLPRFLELGLLDPNDPHKPEEPVPGFPHLKVADIKRAYSHIQESKPILEKMVKETKASSGVIWNSAKELEESELETIWREIPAPSFLLPLPKHYRASSTSLLDTDRSTFQWLDQQPPGSVLYVGFGSQSSLDPEDFLALAEGLVLSGQPFLWLVRPGFVKGYEWIEALPDGYLGPQGRIVKSAPQQEVLAHGAIGAFWTHGGWNGTMESVCEGVPMIFSDFGLDQPLNARYMSEKLKIGVYLDNGFVKEKIVEAINLVMVDEKGAPYRANAQKLKDKLDRSIAPGGSSYESLERLQSYISSL

SEQ ID NO: 378 - protein, 457 aa, synthetic construct
MPNTNTTTRRRRRVILFPVPFPGHLNPMLQLANVLYSRGFEITILYTNFNKPKTSNYPHFQFRPILDNDPQPEWLRNLPTHGPGVGARIPVINKHGADEFRAALEQCIADTESDEEVACLITDALWYFAQPVADSLNLRRLVLQTGSLFNFHCLVCLPRFLELGLLDPDDPHKPDEPVPGFPHLKVADIKRAYSHIQESKPILEKMVKETRASSGVIWNSAKELEESELETIWREIPAPSFLLPLPKHYRASSTSLLDTDRSTFQWLDTQPPGSVLYVGFGSQSSLDPEEFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMSEKLKVGVYLDNGFIPDKIAEAIKLVMVEERGEPYRQNAKALKDKLDRSIMPGGSSYESLESLQSYISSL

SEQ ID NO: 379 - protein, 457 aa, synthetic construct
MPNTNPTTVRRRRIILFPVPFPGHLNPMLQLANVLYRRGFEITILYTNFNAPKTSLYPHFTFRQILDNDPQPEWLRNLPTHGPGVGARIPVINKHGADELRAALLACIADTESDEEVACLITDALWYFAQPVADSLNLRRLVLQTGSLFNFHCLVSLPKFLELGLLDPNDPHRDDEPVPGFPHLKVADIKRAYSHIQESKPILEKMVKETRASSGVIWNSAKELEESELETIWREIPAPSFLLPLPKHYRASSTSLLDTDRSTFQWLDTQPPESVLYVGFGSQSELDPEDFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMTEKLKVGVYLDNGFVKDKIAEAINLVMEEERGEEYRANAQKLKDKLDRSIAPGGSSYESLERLQSYISSL

SEQ ID NO: 380 - protein, 457 aa, synthetic construct
MPNTNPTTRRRRRVVMFPVPFPGHLNPMLQLANVLYRRGFEITILHTNFNKPKTSNYPHFQFRPILDNDPQPEWLRNLPTTGPGVGARIPVINKHGADRFEEALEECIAETESDEEVACLITDALWYFAQPVADSLGLRRLVLQTGSLFNFHCLVSLPEFLELGLLDPDDPHKDDEPVPGFPHLKVADIKRAYSHIQESKPILEKMVKETKASSGVIWNSFKELEESELETIWREIPAPSFLLPLPKHYRASSTSLLDTDRSTAAWLDTQPAESVLYVGFGSQSSLEPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMSEKLKIGVYLDDGFVKEKIVEAINLVMEEERGAPYRQNAQKLKQKLDRSIAPGGSSYESLERLQSYISSL

SEQ ID NO: 381 - protein, 457 aa, synthetic construct
MPNTNPTTRRRRRIILFPVPFPGHLNPMLQLANVLYRRGFEITILHTNFNKPKTSLYPHFQFRQILDNDPQPEWLRNLPTHGPGVGARIPVINKHGADEFKEALEALIAETESDEEVACLITDALWYFAQEVADSLNLRRLVLQTGSLFNFHCLVSLPKFLELGLLDPNDPHKDEEPVPGFPHLKVADIKRAYSHIQESKPILEKMVKETKASSGVIWNSAKELEESELETIWREIPAPSFLLPLPKHYRASSTSLLDTDRSVFQWLDTQPAGSVLYVGFGSQSSLDPEEFLALADGLVLSGQPFLWLVRPGFVKGYEWIEALPDGYLGPKGRIVKSAPQQEVLAHGAIGAFWTHGGWNGTMESVCEGVPMIFSDFGLDQPLNARYMTEKLKVGVYLDNGFVKDAIAEAINLVMVEERGEPYRQNAKALKDKLDRSIAPGGSSYESLESLQSYISSL

SEQ ID NO: 382 - protein, 457 aa, synthetic construct
MPNTNTTTRRRRRVILFPVPFPGHLNPMLQLANVLYSRGFEITILYTNFNKPKTSNYPHFQFRPILDNDPQPEWLRNLPTHGPGVGARIPVINKHGAEEFRAALEALIADTESDEEVACLITDALWYFAQPVADSLNLRRLVLQTGSLFNFHCLVSLPRFLELGLLDPDDTTRDEEPVPGFPHLKVADIKRAYSHIQESKPILEKMVKETRASSGVIWNSAKELEESELETIWREIPAPSFLLPLPKHYRASSTSLLDTDRSTFQWLDTQPPGSVLYVGFGSQSSLDPAEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSDKLKVGVYLDNGWVKEAIAEAINLVMKEERGEVYRQNAKALKDKLDRSIAPGGSSYESLERLQSYISSL

SEQ ID NO: 383 - protein, 457 aa, synthetic construct
MPNTNTTTRRRRRVVLFPVPFPGHLNPMLQLANVLYSRGFEITILYTNFNAPKTSNYPHFQFVPILDNDPQPEWLRNLPTTGPGVGARIPVINKHGADELRAALEECIAETESDEEVACLITDALWYFAQPVADSLNLRRLVLQTGSLFNFHCLVSLPRFLELGLLDPNDPHRDEEPVPGFPHLKVADIKRAYSHIQESKPILMKMVEETRKSSGVIWNSAKELEESELETIWREIPAPSFLLPLPKHYRASSTSLLDTDRSTFQWLDTQPAGSVLYVGFGSQSSLDPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDDGFVKDKIAEAINEVMKEERGKVYRANAKKLKDKLDRSIAPGGSSYESLERLQSYISSL

SEQ ID NO: 384 - protein, 457 aa, synthetic construct
MPNTNPTVRRRRRIIMFPVPFPGHLNPMLQLANVLYRRGFEITILYTNFNKPKTSNYPHFTFRPILDNDPQPEWLRNLPTTGPGVGARIPVINKHGADEFRAALLQLIAETESDEEVACLITDALWYFAQPVADSLGLRRLVLQTGSLFNFHCLVSLPRFLELGLLDPDDPHKLDEPVPGFPHLRVADIRRAYSHIQESKPILEKMVKETKASSGVIWNSAKELEESELETIWREIPAPSFLIPLPKHYRASSTSLLDTDRSTAAWLDTQPAGSVLYVGFGSQSELEPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMTDKLKIGVYLDDGFVKDKIVEAINLVMKEERGAPYRQNARALKDKLDRSIAPGGSSYESLERLQSYISSL

SEQ ID NO: 385 - protein, 457 aa, synthetic construct
MPNTNPTTRRRRRVILFPVPFPGHLNPMLQLANVLYRRGFEITILYTNFNKPKTSNYPHFQFRPILDNDPQDEWLRNLPTHGPGVGARIPVINKHGADEFKAALEALIAETESDEEVACLITDALWYFAQPVADELGLRRLVLQTGSLFNFHCLVSLPKFLELGLLDPDDKHKDDEPAPGFPHLKVADIKRAYSHIQESKPILEKMVKETKASSGVIWNSFKELEESELETIWREIPAPSFLLPLPKHYRASSTSLLDTDRSTAQWLDTQPAGSVLYVGFGSQSSLEPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMSDKLKVGVYLDDGFVKDAIAEAINLVMVEEKGEPYRANAKKLKDKLDRSIAPGGSSYESLERLQSYISSL

SEQ ID NO: 386 - protein, 457 aa, synthetic construct
MPNTNPTTRRRRRVILFPVPFPGHLNPMLQLANVLYRRGFEITILYTNFNKPKTSNYPHFQFRQILDNDPQPEWLRNLPTTGPGVGARIPVINKHGADEFRAALEECIAETESDEEVACLITDALWYFAQPVADSLGLRRLVLQTGSLFNFHCLVSLPRFLELGLLDPDDPHRDDEPVPGFPHLKVADIKRAYSHIQESKPILEKMVKETKASSGVIWNSFKELEESELETIWREIPAPSFLLPLPKHYRASSTSLLDTDRSTAAWLDQQPAGSVLYVGFGSQSSLDPAEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSDKLKIGVYLDDGFVKDKIAEAINLVMVEERGEPYRQNAKVLKQKLDRSIAPGGSSYESLESLQSYISSL

SEQ ID NO: 387 - protein, 457 aa, synthetic construct
MPNTNTTTVRRRRVILFPVPFPGHLNPMLQLANVLYRRGFEITILYTNFNKPKTSLYPHFQFRPILDNDPQPEWLRNLPTTGPGVGARIPVINKHGADQFREALEACIADTESDEEVACLITDALWYFAQPVADSLNLRRLVLQTGSLFNFHCLVSLPRFLELGLLDPNDKTKPDEPVPGFPHLKVKDIRRAYSHIQESKPILEKMVEETRASSGVIWNSAKELEESELETIWREIPAPSFLLPLPKHYRASSTSLLDTDRSTFAWLDTQPAGSVLYVGFGSQSSLEPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDNGFVKDKIAEAINLVMEEEKGEPYRANAQKLKQKLDRSIAPGGSSYESLERLQSYISSL

SEQ ID NO: 388 - protein, 457 aa, synthetic construct
MPNTNTTTRRRRRIILFPVPFPGHLNPMLQLANVLYRRGFEITILYTNFNKPKTSNYPHFQFVPILDNDPQPEWLRNLPTTGPGVGARIPVINKHGADEFEAALEACIAETESDEEVACLITDALWYFAQPVADSLGLRRLVLQTGSLFNFHCLVSLPRFLELGLLDPNTPHKPDEPVPGFPHLKVADIKRAYSHIQESKPILEKMVKETKASSGVIWNSAKELEESELETIWREIPAPSFLIPLPKHYRASSTSLLDTDRSTFAWLDTQPPRSVLYVGFGSQSSLDPEEFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMTEKLKVGVYLDDGFIKDRIAEAINTVMEEEKGEPYRANAKALKQKLDRSIAPGGSSYESLERLQSYISSL

SEQ ID NO: 389 - protein, 457 aa, synthetic construct
MPNTNPTTRRRRRIILFPVPFPGHLNPMLQLANVLYSRGFEITILHTNFNKPKTSNYPHFQFRQILDNDPQDEWLRNLPTHGPGVGARIPVINKHGADEFEAALEALIAETESDEEVACLITDALWYFAQPVADSLGLRRLVLQTGSLFNFHCLVSLPRFLELGLLDPDDPTKDDEPVPGFPHLKVADIKRAYSHIQESKPILEKMVKETRASSGVIWNSAKELEESELETIWREIPAPSFLIPLPKHYRASSTSLLDTDRSTFQWLDQQPPGSVLYVGFGSQSSLDPEEFLALADGLVLSGQPFLWLVRPGFVKGYEWIEALPDGYLGEQGRIVKSAPQQEVLAHPAIGAFWTHGGWNGTMEAVCEGVPMIFSDFGLDQPLNARYMSDKLKIGVYLDNGFVKEKIAEAINLVMEAERGEPYRANAKALKDKLDRSIAPGGSSYESLERLQSYISSL

SEQ ID NO: 390 - protein, 457 aa, synthetic construct
MPNTNPTTRRRRRVILFPVPFPGHLNPMLQLANVLYRRGFRITILYTNFNKPKTSNYPHFQFVPILDNDPQPEWLRNLPTTGPGVGARIPVINKHGADEFREALEKCIADTESDEEVACLITDALWYFAQPVADSLGLRRLVLQTGSLFNFHCLVSLPRFLELGLLDPNDKHKDEEPVPGFPHLRVADIKRAYSHIQESKPILEKMVKETRASSGVIWNSAKELEESELETIWREIPAPSFLLPLPKHYRASSTSLLDTDRSTFQWLDTQPPGSVLYVGFGSQSSLEPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMSEKLKIGVYLDDGFVKEAIVEAINLVMEEEKGEPYRQNAQVLKDKLDRSIAPGGSSYESLERLQSYISSL

SEQ ID NO: 391 - protein, 457 aa, synthetic construct
MPNTNPTTRRRRRIVLFPVPFPGHLNPMLQLANVLYRRGFEITILYTNFNKPKTSLYPHFQFVPILDNDPQPEWLRNLPTTGPGVGARIPVINKHGAEEFRAALEALIAETESDEEVACLITDALWYFAQPVADSLNLRRLVLQTGSLFNFHCLVSLPRFLELGLLDPDDTHKDDEPAPGFPHLKVADIRRAYSHIQESKPILEKMVKETRASSGVIWNSAKELEESELETIWREIPAPSFLLPLPKHYRASSTSLLDTDPSTAQWLDTQPPGSVLYVGFGSQSSLDPEDFLALADGLVLSGQPFLWLVRPGFVKGHEWIEALPDGYLGEKGRIVKSAPQQEVLAHPAIGAFWTHGGWNGTMEAVCEGVPMIFSDFGLDQPLNARYMSEKLKIGVYLDDGFVPDKIVEAINLVMKEEKGEEYRQNAQALKDKLDRSIAKGGSSYESLERLQSYISSL

SEQ ID NO: 392 - protein, 457 aa, synthetic construct
MPNTNTTTRRRRRIIMFPVPFPGHLNPMLQLANVLYRRGFEITILYTNFNKPKTSNYPHFQFVTILDNDPQPEWLRNLPTTGPGVGARIPVINKHGAEQFKEALEKLIAETESDEEVACLITDALWYFAQPVADELGLRRLVLQTGSLFNFHCLVSLPRFLELGLLDPDTPHRDDEPVPGFPHLRVADIKRAYSHIQESKPILEKMVKETRASSGVIWNSAKELEESELETIWREIPAPSFLLPLPKHYRASSTSLLDTDRSTFAWLDTQPAGSVLYVGFGSQSSLDPEEFLALADGLVLSGQPFLWLVRPGFVKGHEWIEALPDGYLGPKGRIVKSAPQQEVLAHGAIGAFWTHGGWNGTMEAVCEGVPMIFSDFGLDQPLNARYMSEKLKIGVYLEDGFIKEAIAEAINEVMKEERGAPMRQNAKKLKDKLDRSIMPGGSSYESLERLQSYISSL

SEQ ID NO: 393 - protein, 457 aa, synthetic construct
MPNTNTTTRRRRRVILFPVPFPGHLNPMLQLANVLYSRGFEITILYTNFNAPKTSNYPHFQFRQILDNDPQPEWLRNLPTTGPGVGARIPVINKHGADELREALEALIAETESDEEVACLITDALWYFAQPVADSLGLRRLVLQTGSLFNFHCLVSLPRFLELGLLDPDDPHKPDEPVPGFPHLKVADIKRAYSHIQESKPILEKMVKETKASSGVIWNSFKELEESELETIWREIPAPSFLLPLPKHYRASSTSLLDTDRSTFAWLDTQPAGSVLYVGFGSQSELDPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSDKLKIGVYLDNGFVKADIAEAIKLVMEEERGAPYRQNAQALKDKLDRSIAPGGSSYESLERLQSYISSL

SEQ ID NO: 394 - protein, 457 aa, synthetic construct
MPNTNPTTRRRRRVILFPVPFPGHLNPMLQLANVLYRRGFEITILYTNFNKPKTSNYPHFQFRPILDNDPQPEWLRNLPTTGPGVGARIPVINKHGADEFKEALLQCIAETESDEEVACLITDALWYFAQPVADSLNLRRLVLQTGSLFNFHCLVSLPKFLELGLLDPNDPHRPEEPVPGFPHLKVADIKRAYSHIQESKPILEKMVKETRASSGVIWNSAKELEESELETIWREIPAPSFLLPLPKHYRASSTSLLDTDRSTFQWLDQQPARSVLYVGFGSQSELEPEEFLALAEGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKVGVYLDDGFVKDAIAEAINLVMEEERGEPYRQNAQALKDKLDRSIAPGGSSYESLERLQSYISSL

SEQ ID NO: 395 - protein, 457 aa, synthetic construct
MPNTNPTTRRRRRVILFPVPFPGHLNPMLQLANVLYRRGFEITILYTNFNKPKTSNYPHFQFRPILDNDPQPEWLRNLPTTGPGVGARIPVINKHGAEELREALLKLIAETESDEEVACLITDALWYFAQPVADSLNLRRLVLQTGSLFNFHCLVSLPRFLELGLLDPNDPHKDDEPAPGFPHLKVADIRRAYSHIQESKPILEKMVEETKASSGVIWNSAKELEESELETIWREIPAPSFLLPLPKHYRASSTSLLDTDRSTFQWLDTQPPESVLYVGFGSQSSLDPAEFLALADGLVLSGQPFLWLVRPGFVKGKEWIEALPDGYLGEKGRIVKSAPQQEVLAHPAIGAFWTHGGWNGTMEAVCEGVPMIFSDFGLDQPLNARYMTEKLKVGVYLDDGFVKDKIVAAINLVMKDEKGAPYRANAKKLKDKLDRSIAPGGSSYESLERLQSYISSL

SEQ ID NO: 396 - protein, 457 aa, synthetic construct
MPNTNTTTRRRRRIVMFPVPFPGHLNPMLQLANVLYRRGFEITILYTNFNKPKTSNYPHFQFRPILDNDPQPEWLRNLPTTGPGVGARIPVINKHGADELREALEECIAETESDEEVACLITDALWYFAQPVADSLGLRRLVLQTGSLFNFHCLVSLPRFLELGLLDPDTTHKPDEPVPGFPHLKVADIKRAYSHIQESKPILEKMVKETRASSGVIWNSAKELEESELETIWREIPAPSFLLPLPKHYRASSTSLLDTDRSTFQWLDQQPAGSVLYVGFGSQSSLEPEEFLALADGLVLSGQPFLWLVRPGFVKGYEWIEALPDGYLGEKGRIVKSAPQQEVLAHGAIGAFWTHGGWNGTMESVCEGVPMIFSDFGLDQPLNARYMTEKLKIGVYLDDGFIKEDIVEAIKLVMKEERGEEYRANAQKLKQKLDRSIMPGGSSYESLERLQSYISSL

SEQ ID NO: 397 - protein, 457 aa, synthetic construct
MPNTNTTVRRRRRIILFPVPFPGHLNPMLQLANVLYRRGFEITILYTNFNAPKTSNYPHFQFRQILDNDPQPEWLRNLPTTGPGVGARIPVINKHGADELRAALEALIAETESDEEVACLITDALWYFAQPVADSLGLRRLVLQTGSLFNFHCLVSLPEFLELGLLDPDDPTRDDEPVPGFPHLKVADIKRAYSHIQESKPILEKMVKETRASSGVIWNSAKELEESELETIWREIPAPSFLLPLPKHYRASSTSLLDTDRSTFQWLDTQPAGSVLYVGFGSQSSLEPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSDKLKVGVYLDNGFVKDAIVEAINLVMEDERGEPYRANAQKLKQKLDRSIAPGGSSYESLESLQSYISSL

SEQ ID NO: 398 - protein, 457 aa, synthetic construct
MPNTNTTTRRRRRVILFPVPFPGHLNPMLQLANVLYRRGFRITILYTNFNKPKTSNYPHFQFRPILDNDPQPEWLRNLPTTGPGVGARIPVINKHGADEFKEALEALIAETESDEEVACLITDALWYFAQPVADSLNLRRLVLQTGSLFNFHCLVSLPKFLELGLLDPNDPHKDDEPVPGFPHLKVADIKRAYSHIQESKPILEKMVKETKASSGVIWNSAKELEESELETIWREIPAPSFLLPLPKHYRASSTSLLDTDPSVFQWLDQQPAGSVLYVGFGSQSELEPEEFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMSEKLKVGVYLDDGWIKEKIAEAINLVMVEEKGEVYRQNAKALKDKLDRSIAPGGSSYESLERLQSYISSL

SEQ ID NO: 399 - protein, 457 aa, synthetic construct
MPNTNPTTRRRRRVIMFPVPFPGHLNPMLQLANVLYRRGFEITILHTNFNAPKTSNYPHFQFRPILDNDPQPEWLRNLPTTGPGVGARIPVINKHGADEFRAALLECIAETESDEEVACLITDALWYFAQPVADSLNLRRLVLQTGSLFNFHCLVSLPRFLELGLLDPDSKHKDDEPVPGFPHLKVADIKRAYSHIQESKPILMKMVKETKASSGVIWNSFKELEESELETIWREIPAPSFLLPLPKHYRASSTSLLDTDRSTAQWLDQQPAGSVLYVGFGSQSSLDPEDFLALADGLVLSGQPFLWLVRPGFVKGYEWIEALPDGYLGPKGRIVKSAPQQEVLAHPAIGAFWTHGGWNGTMESVCEGVPMIFSDFGLDQPLNARYMTEKLKVGVYLDDGFVKDRIVEAINLVMKAERGAEYRANAQKLKQKLDRSIAPGGSSYESLERLQSYISSL

SEQ ID NO: 400 - protein, 457 aa, synthetic construct
MPNTNTTTRRRRRIVMFPVPFPGHLNPMLQLANVLYRRGFEITILYTNFNKPKTSNYPHFQFRPILDNDPQPEWLRNLPTTGPGVGARIPVINKHGAEELKEALEALIAETESDEEVACLITDALWYFAQPVADSLNLRRLVLQTGSLFNFHCLVSLPRFLELGLLDPDDPHKDEEPVPGFPHLRVADIRRAYSHIQESKPILEKMVKETKASSGVIWNSAKELEESELETIWREIPAPSFLLPLPKHYRASSTSLLDTDRSTFAWLDTQPAGSVLYVGFGSQSSLEPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEILKIGVYLDNGFVKEKIAEAINLVMEEPRGAPYRQNAQALKDKLDRSIMPGGSSYESLERLQSYISSL

SEQ ID NO: 401 - protein, 457 aa, synthetic construct
MPNTNPTTRRRRRIILFPVPFPGHLNPMLQLANVLYRRGFEITILYTNFNKPKTSNYPHFQFRPILDNDPQPEWLRNLPTTGPGVGARIPVINKHGAEEFREALEALIAETESDEEVACLITDALWYFAQPVADSLGLRRLVLQTGSLFNFHCLVSLPRFLELGLLDPDDKHAPEEPVPGFPHLRVADIKRAYSHWQESKPILEKMVEETKASSGVIWNSFKELEESELETIWREIPAPSFLLPLPKHYRASSTSLLDTDRSVFQWLDTQPPGSVLYVGFGSQSELEPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKIGVYLDDGFVKEKIAEAINEVMEEERGAPYRQNAQALKDKLDRSIAPGGSSYESLESLQSYISSL

SEQ ID NO: 402 - protein, 457 aa, synthetic construct
MPNTNPTTRRRRRVIMFPVPFPGHLNPMLQLANVLYRRGFEITILYTNFNAPKTSNYPHFQFRPILDNDPQPEWLRNLPTTGPGVGARIPVINKHGADEFREALEECIAETESDEEVACLITDALWYFAQPVADSLNLRRLVLQTGSLFNFHCLVSLPRFLELGLLDPDDTHRPEEPVPGFPILKVADIKRAYSHIQESKPILEKMVKETKASSGVIWNSAKELEESELETIWREIPAPSFLIPLPKHYRASSTSLLDTDRSTFQWLDTQPAGSVLYVGFGSQSELDPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLENGFVKEKIAEAINLVMVEEKGEPYRANAKALKDKLDRSIAPGGSSYESLERLQSYISSL

SEQ ID NO: 403 - protein, 457 aa, synthetic construct
MPNTNPTTRRRRRVIMFPVPFPGHLNPMLQLANVLYRRGFEITILYTNFNKPKTSNYPHFQFRPILDNDPQPEWLRNLPTHGPGVGARIPVINKHGADEFRAALEKCIADTESDEEVACLITDALWYFAQPVADSLGLRRLVLQTGSLFNFHCLVSLPRFLELGLLDPDDPLKDDEPVPGFPHLKVADIKRAYSHIQESKPILEKMVKETKASSGVIWNSAKELEESELETIWREIPAPSFLLPLPKHYRASSTSLLDTDRSTAQWLDTQPAESVLYVGFGSQSSLDPEEFLALADGLVLSGQPFLWLVRPGFVKGYEWIEALPDGFLGPKGRIVKSAPQQEVLAHPAIGAFWTHGGWNGTMEAVCEGVPMIFSDFGLDQPLNARYMSEKLKVGVYLDDGFVKDKIVAAINLVMEEEKGKPYRANAQALKDKLDRSIAPGGSSYESLERLQSYISSL

SEQ ID NO: 404 - protein, 457 aa, synthetic construct
MPNTNTTTRRRRRIVMFPVPFPGHLNPMLQLANVLYRRGFEITILYTNFNKPKTSNYPHFQFVPILDNDPQPEWLRNLPTTGPGVGARIPVINKHGADQFKQALLECIAETESDEEVACLITDALWYFAQPVADSLGLRRLVLQTGSLFNFHCLVSLPRFLELGLLDPDDPTKDEEPAPGFPHLRVADIKRAYSHIQESKPILEKMVKETKASSGVIWNSAKELEESELETIWREIPAPSFLLPLPKHYRASSTSLLDTDRSTFQWLDTQPARSVLYVGFGSQSSLDPEEFLALADGLVLSGQPFLWLVRPGFVKGYEWIEALPDGYLGPRGRIVKSAPQQEVLAHPAIGAFWTHGGWNGTMESVCEGVPMIFSDFGLDQPLNARYMSEKLKIGVYLDDGWVKEKIAEAINLVMEEEKGKEYRQNAQALKDKLDRSIAPGGSSYESLERLQSYISSL

SEQ ID NO: 405 - protein, 457 aa, synthetic construct
MPNTNTTTRRRRRIILFPVPFPGHLNPMLQLANVLYRRGFEITILHTNFNKPKTSNYPHFQFRPILDNDPQPEWLRNLPTTGPGVGARIPVINKHGADELKEALEELIAETESDEEVACLITDALWYFAQPVADELGLRRLVLQTGSLFNFHCLVSLPRFLELGLLDPDDTTKDDEPAPGFPHLKVADIKRAYSHIQESKPILEKMVKETKASSGVIWNSAKELEESELETIWREIPAPSFLLPLPKHYRASSTSLLDTDRSTFQWLDQQPPGSVLYVGFGSQSSLELEEFLALADGLVLSGQPFLWLVRPGFVKGYEWIEALPDGYLGEQGRIVKSAPQQEVLAHGAIGAFWTHGGWNGTMESVCEGVPMIFSDFGLDQPLNARYMSEKLKIGVYLDNGFVKDDIAEAINLVMEEERGAEYRANAQVLKDKLDRSIAPGGSSYESLESLQSYISSL

SEQ ID NO: 406 - protein, 457 aa, synthetic construct
MPNTNTTTRRRRRVVLFPVPFPGHLNPMLQLANVLYRRGFEITILYTNFNKPKTSNYPHFQFRPILDNDPQPEWLRNLPTHGPGVGARIPVINKHGAEEFREALEACIADTESDEEVACLITDALWYFAQPVADSLNLRRLVLQTGSLFNFHCLVSLPRFLELGLLDPDDPHKDDEPVPGFPMLKVADIRRAYSHIQESKPILEKMVKETKASSGVIWNSAKELEESELETIWREIPAPSFLLPLPKHYRASSTSLLDTDRSTFQWLDTQPPGSVLYVGFGSQSSLEPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKIGVYLDDGFVKEKIAEAINLVMVAEKGEPYRQNAKKLKDKLDRSIAPGGSSYESLESLQSYISSL

SEQ ID NO: 407 - protein, 457 aa, synthetic construct
MPNTNPTTRRRRRVVLFPVPFPGHLNPMLQLANVLYRRGFEITILYTNFNKPKTSNYPHFQFRPILDNDPQPEWLRNLPTTGPGVGARIPVINKHGAEEFEAALEQCIADTESDEEVACLITDALWYFAQPVADSLNLRRLVLQTGSLFNFHCLVCLPRFLELGLLDPDTTTKDDEPVPGFPMLKVADIKRAYSHIQESKPILEKMVEETKASSGVIWNSAKELEESELETIWREIPAPSFLLPLPKHYRASSTSLLDTDRSVFQWLDTQPAGSVLYVGFGSQSSLDPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKIGVYLDDGFVKDKIVEAINEVMEEERGEVYRANAQKLKDKLDRSIAPGGSSYESLESLQSYISSL

SEQ ID NO: 408 - protein, 457 aa, synthetic construct
MPNTNTTTRRRRRIVMFPVPFPGHLNPMLQLANVLYSRGFEITILYTNFNKPKTSNYPHFQFRQILDNDPQPEWLRNLPTHGPGVGARIPVINKHGADEFEAALLQCIAETESDEEVACLITDALWYFAQEVADSLGLRRLVLQTGSLFNFHCLVSLPRFLELGLLDPDDPHKDDEPVEGFPHLKVADIKRAYSHIQESKPILEKMVKETKASSGVIWNSAKELEESELETIWREIPAPSFLIPLPKHYRASSTSLLDTDRSTAQWLDTQPAGSVLYVGFGSQSSLDPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMTEKLKIGVYLDDGFIKDDIAEAINLVMEEEKGEPYRANAKKLKDKLDRSIAKGGSSYESLERLQSYISSL

SEQ ID NO: 409 - protein, 457 aa, synthetic construct
MPNTNTTTRRRRRVIMFPVPFPGHLNPMLQLANVLYRRGFEITILHTNFNKPKTSNYPHFQFRPILDNDPQDEWLRNLPTTGPGVGARIPVINKHGAEEFREALEECIAETESDEEVACLITDALWYFAQPVADSLGLRRLVLQTGSLFNFHCLVCLPRFLELGLLDPDDKHKDDEPVPGFPHLKVADIKRAYSHIQESKPILEKMVKETKASSGVIWNSAKELEESELETIWREIPAPSFLLPLPKHYRASSTSLLDTDRSTFAWLDQQPAGSVLYVGFGSQSSLDPEEFLALADGLVLSGQPFLWLVRPGFVKGYEWIEALPDGYLGPQGRIVKSAPQQEVLAHPAIGAFWTHGGWNGTMESVCEGVPMIFSDFGLDQPLNARYMTEKLKVGVYLDDGFVKDDIAEAINLVMEAEKGEPYRANAQALKDKLDRSIAPGGSSYESLESLQSYISSL

SEQ ID NO: 410 - protein, 457 aa, synthetic construct
MPNTNTTTVRRRRVILFPVPFPGHLNPMLQLANVLYRRGFRITILYTNFNKPKTSNYPHFQFVPILDNDPQPEWLRNLPTHGPGVGARIPVINKHGADEFRAALLKLIADTESDEEVACLITDALWYFAQPVADSLNLRRLVLQTGSLFNFHCLVSLPRFLELGLLDPDDPHRPDEPVPGFPMLKVADIKRAYSHIQESKPILMKMVKETKASSGVIWNSFKELEESELETIWREIPAPSFLLPLPKHYRASSTSLLDTDRSTFQWLDTQPAGSVLYVGFGSQSSLEPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDDGFVKDKIVEAINLVMEEERGEPYRQNAKVLKQKLDRSIAPGGSSYESLERLQSYISSL

SEQ ID NO: 411 - protein, 457 aa, synthetic construct
MPNTNTTTRRRRRIILFPVPFPGHLNPMLQLANVLYRRGFEITILYTNFNKPKTSNYPHFQFRPILDNDPQPEWLRNLPTTGPGVGARIPVINKHGAERLREALEALISETESDEEVACLITDALWYFAQPVADELGLRRLVLQTGSLFNFHCLVSLPKFLELGLLDPNDPHKDDEPVPGFPHLKVADIRRAYSHIQESKPILEKMVKETRASSGVIWNSAKELEESELETIWREIPAPSFLLPLPKHYRASSTSLLDTDPSTFQWLDTQPPGSVLYVGFGSQSSLDPEDFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDDGFVKDKIAEAINLVMKEEKGKVYRANAQKLKQKLDRSIAPGGSSYESLERLQSYISSL

SEQ ID NO: 412 - protein, 457 aa, synthetic construct
MPNTNTTVRRRRRIVLFPVPFPGHLNPMLQLANVLYSRGFEITILYTNFNKPKTSNYPHFQFRPILDNDPQPEWLRNLPTTGPGVGARIPVINKHGAEEFKEALEACIAETESDEEVACLITDALWYFAQPVADSLGLRRLVLQTGSLFNFHCLVSLPKFLELGLLDPDDPHKDEEPVPGFPHLRVADIKRAYSHIQESKPILEKMVKETKASSGVIWNSFKELEESELETIWREIPAPSFLIPLPKHYRASSTSLLDTDRSTAAWLDTQPPGSVLYVGFGSQSSLDPEEFLALADGLVLSGQPFLWLVRPGFVKGYEWIEALPDGFLGPRGRIVKSAPQQEVLAHGAIGAFWTHGGWNGTMEAVCEGVPMIFSDFGLDQPLNARYMTEKRKIGVYLDDGFVKEKIAEAINLVMEEEKGKPYRANAQKLKDKLDRSIAPGGSSYESLERLQSYISSL

SEQ ID NO: 413 - protein, 457 aa, synthetic construct
MPNTNTTTRRRRRIILFPVPFPGHLNPMLQLANVLYRRGFEITILYTNFNKPKTSNYPHFQFRPILDNDPQPEWLRNLPTTGPGVGARIPVINKHGAEELREALEELIAETESDEEVACLITDALWYFAQPVADSLNLRRLVLQTGSLFNFHCLVSLPRFLELGLLDPNDTHKDDEPVPGFPHLKVADIKRAYSHIQESKPILEKMVKETKASSGVIWNSFKELEESELETIWREIPAPSFLLPLPKHYRASSTSLLDTDRSTFAWLDTQPPGSVLYVGFGSQSSLDPEEFLAIADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMTEKLKIGVYLDNGFVKERIAEAIHLVMEEEKGEVYRANAKKLKDKLDRSIAPGGSSYESLESLQSYISSL

SEQ ID NO: 414 - protein, 457 aa, synthetic construct
MPNTNPTTRRRRRIILFPVPFPGHLNPMLQLANVLYSRGFEITILYTNFNKPKTSLYPHFQFRPILDNDPQDEWLRNLPTHGPGVGARIPVINKHGADQFKEALEACIADTESDEEVACLITDALWYFAQPVADSLNLRRLVLQTGSLFNFHCLVCLPEFLELGLLDPDDPTKLDEPVPGFPHLKVADIKRAYSHIQESKPILEKMVKETKASSGVIWNSFKELEESELETIWREIPAPSFLLPLPKHYRASSTSLLDTDRSTFQWLDTQPAGSVLYVGFGSQSELDPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSEKLKVGVYLDNGFVKEAIAEAINLVMVEEKGEPYRQNAKVLKDKLDRSIAPGGSSYESLESLQSYISSL

SEQ ID NO: 415 - protein, 457 aa, synthetic construct
MPNTNTTTRRRRRIIMFPVPFPGHLNPMLQLANVLYRRGFEITILHTNFNAPKTSNYPHFQFVPILDNDPQPEWLRNLPTTGPGVGARIPVINKHGAEEFEEALLKCIAETESDEEVACLITDALWYFAQPVADSLNLRRLVLQTGSLFNFHCLVSLPRFLELGLLDPDDKHKDDEPAPGFPHLKVADIRRAYSHIQESKPILEKMVKETRASSGVIWNSAKELEESELETIWREIPAPSFLLPLPKHYRASSTSLLDTDPSTFQWLDTQPAESVLYVGFGSQSELDPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKIGVYLDNGFVPEAIVEAINLVMKEERGKEYRANARKLKDKLDRSIAPGGSSYESLERLQSYISSL

SEQ ID NO: 416 - protein, 457 aa, synthetic construct
MPNTNPTTRRRRRIILFPVPFPGHLNPMLQLANVLYRRGFEITILYTNFNKPKTSNYPHFQFRPILDNDPQPEWLRNLPTTGPGVGARIPVINKHGADELKEALLELIAETESDEEVACLITDALWYFAQPVADELNLRRLVLQTGSLFNFHCLVSLPRFLELGLLDPDDPHRDDEPVPGFPHLKVADIKRAYSHIQESKPILEKMVEETKKSSGVIWNSAKELEESELETIWREIPAPSFLIPLPKHYRASSTSLLDTDRSTAQWLDQQPAGSVLYVGFGSQSSLEPEEFLALADGLVLSGQPFLWLVRPGFVKGYEWIEALPDGYLGPKGRIVKSAPQQEVLAHGAIGAFWTHGGWNGTMEAVCEGVPMIFSDFGLDQPLNARYMTEKLKVGVYLDDGFIKDKIAAAINLVMVEERGKPMRANAKALKDKLDRSIMPGGSSYESLESLQSYISSL

SEQ ID NO: 417 - protein, 457 aa, synthetic construct
MPNTNPTTRRRRRIILFPVPFPGHLNPMLQLANVLYRRGFEITILYTNFNKPKTSNYPHFQFRQILDNDPQPEWLRNLPTTGPGVGARIPVINKHGADEFKEALEALIAETESDEEVACLITDALWYFAQPVADSLGLRRLVLQTGSLFNFHCLVCLPRFLELGLLDPDDKHRDEEPVPGFPHLKVADIKRAYSHIQESKPILEKMVKETKASSGVIWNSFKELEESELETIWREIPAPSFLLPLPKHYRASSTSLLDTDRSTFQWLDQQPAGSVLYVGFGSQSSLDPEEFLALADGLVLSGQPFLWLVRPGFVKGYEWIEALPDGYLGPKGRIVKSAPQQEVLAHPAIGAFWTHGGWNGTMEAVCEGVPMIFSDFGLDQPLNARYMSEKLKVGVYLDNGFVPDAIVEAIHLVMVEEKGEVYRQNAQALKDKLDRSIAPGGSSYESLERLQSYISSL

SEQ ID NO: 418 - protein, 457 aa, synthetic construct
MPNTNTTTRRRRRVILFPVPFPGHLNPMLQLANVLYSRGFRITILYTNFNKPKTSNYPHFQFRPILDNDPQPEWLRNLPTTGPGVGARIPVINKHGAEEFREALEALIAETESDEEVACLITDALWYFAQPVADSLGLRRLVLQTGSLFNFHCLVCLPRFLELGLLDPDDKHKDEEPVPGFPHLKVADIKRAYSHWQESKPILEKMVKETRASSGVIWNSAKELEESELETIWREIPAPSFLLPLPKHYRASSTSLLDTDRSTFQWLDTQPANSVLYVGFGSQSELDPAEFLALADGLVLSGQPFLWLVRPGFVKGYEWIEALPDGYLGPQGRIVKSAPQQEVLAHPAIGAFWTHGGWNGTMEAVCEGVPMIFSDFGLDQPLNARYMSEKLKVGVYLDNGFVKDAIVEAINLVMKEEKGEPYRANAKVLKDKLDRSIAPGGSSYESLERLQSYISSL

SEQ ID NO: 419 - protein, 457 aa, synthetic construct
MPNTNTTTRRRRRVILFPVPFPGHLNPMLQLANVLYRRGFEITILYTNFNAPKTSNYPHFQFRPILDNDPQPEWLRNLPTHGPGVGARIPVINKHGAEELREALEKCIAETESDEEVACLITDALWYFAQPVADSLNLRRLVLQTGSLFNFHCLVSLPRFLELGLLDPDDTTRDDEPVPGFPHLKVKDIRRAYSHIQESKPILEKMVKETRASSGVIWNSAKELEESELETIWREIPAPSFLLPLPKHYRASSTSLLDTDRSTFQWLDQQPAGSVLYVGFGSQSSLDPEEFLALADGLVLSGQPFLWLVRPGFVKGYEWIEALPDGYLGEQGRIVKSAPQQEVLAHPAIGAFWTHGGWNGTMEAVCEGVPMIFSDFGLDQPLNARYMTEKLKVGVYLDDGFVKEKIVEAINLVMEEERGAEYRQNAQKLKDKLDRSIAPGGSSYESLERLQSYISSL

SEQ ID NO: 420 - protein, 457 aa, synthetic construct
MPNTNPTTRRRRRIILFPVPFPGHLNPMLQLANVLYRRGFEITILYTNFNKPKTSNYPHFQFRPILDNDPQPEWLRNLPTTGPGVGARIPVINKHGADQLEEALEECIAETESDEEVACLITDALWYFAQPVADSLGLRRLVLQTGSLFNFHCLVSLPRFLELGLLDPDSPHRDEEPVPGFPMLRVADIRRAYSHIQESKPILEKMVKETKASSGVIWNSFKELEESELETIWREIPAPSFLLPLPKHYRASSTSLLDTDRSTFAWLDTQPAESVLYVGFGSQSSLEPEEFLALADGLVLSGQPFLWLVRPGFVKGHEWIEALPDGYLGPQGRIVKSAPQQEVLAHPAIGAFWTHGGWNGTMESVCEGVPMIFSDFGLDQPLNARYMTEKLKIGVYLDNGFIKDRIVEAINLVMEEERGEPYRANAKALKDKLDRSIAPGGSSYESLERLQSYISSL

SEQ ID NO: 421 - protein, 457 aa, synthetic construct
MPNTNPTTRRRRRVILFPVPFPGHLNPMLQLANVLYSRGFEITILHTNFNAPKTSNYPHFQFRPILDNDPQPEWLRNLPTTGPGVGARIPVINKHGAEQLREALEALIAETESDEEVACLITDALWYFAQPVADSLGLRRLVLQTGSLFNFHCLVSLPRFLELGLLDPNDKHKDDEPAPGFPHLKVADIKRAYSHIQESKPILEKMVKETKASSGVIWNSAKELEESELETIWREIPAPSFLIPLPKHYRASSTSLLDTDRSTFQWLDTQPAGSVLYVGFGSQSELDPEDFLALADGLVLSGQPFLWLVRPGFVKGYEWIEALPDGYLGPQGRIVKSAPQQEVLAHGAIGAFWTHGGWNGTMEAVCEGVPMIFSDFGLDQPLNARYMTEKLKVGVYLDDGFVKEAIVEAINLVMVEERGEPYRANAKKLKDKLDRSIAPGGSSYESLESLQSYISSL

SEQ ID NO: 422 - protein, 457 aa, synthetic construct
MPNTNPTTRRRRRVILFPVPFPGHLNPMLQLANVLYSRGFEITILYTNFNKPKTSLYPHFTFRPILDNDPQPEWLRNLPTTGPGVGARIPVINKHGAEEFRAALEACIAETESDEEVACLITDALWYFAQPVADSLNLRRLVLQTGSLFNFHCLVSLPRFLELGLLDPNDKTKLDEPVPGFPMLKVADIKRAYSHIQESKPILEKMVKETRASSGVIWNSFKELEESELETIWREIPAPSFLLPLPKHYRASSTSLLDTDRSTFQWLDTQPAGSVLYVGFGSQSSLDPEEFLALADGLVLSGQPFLWLVRPGFVKGYEWIEALPDGYLGEKGRIVKSAPQQEVLAHPAIGAFWTHGGWNGTMEAVCEGVPMIFSDFGLDQPLNARYMSDKLKVGVYLDDGFVKDAIAEAINLVMEEEKGEPYRANAKVLKDKLDRSIMPGGSSYESLERLQSYISSL

SEQ ID NO: 423 - protein, 457 aa, synthetic construct
MPNTNTTTRRRRRVVLFPVPFPGHLNPMLQLANVLYRRGFEITILYTNFNKPKTSLYPHFQFRPILDNDPQPEWLRNLPTTGPGVGARIPVINKHGAEEFREALEALIADTESDEEVACLITDALWYFAQPVADSLNLRRLVLQTGSLFNFHCLVSLPKFLELGLLDPDDPTKDEEPVPGFPHLKVKDIRRAYSHIQESKPILEKMVKETRASSGVIWNSAKELEESELETVWREIPAPSFLLPLPKHYRASSTSLLDTDRSTFQWLDTQPARSVLYVGFGSQSSLDPEEFLALADGLVLSGQPFLWLVRPGFVKGYEWIEALPDGYLGEKGRIVKSAPQQEVLAHGAIGAFWTHGGWNGTMEAVCEGVPMIFSDFGLDQPLNARYMSEKLKIGVYLDDGFVKDKIAEAINEVMEEEKGKEMRANARKLKDKLDRSIAPGGSSYESLERLQSYISSL

SEQ ID NO: 424 - protein, 457 aa, synthetic construct
MPNTNPTTRRRRRIILFPVPFPGHLNPMLQLANVLYSRGFEITILYTNFNKPKTSNYPHFQFRPILDNDPQPEWLRNLPTHGPGVGARIPVINKHGADELKAALLECIAETESDEEVACLITDALWYFAQEVADSLNLRRLVLQTGSLFNFHCLVSLPRFLELGLLDPNDPHKPDEPVPGFPHLRVADIKRAYSHIQESKPILEKMVEETKASSGVIWNSAKELEESELETIWREIPAPSFLIPLPKHYRASSTSLLDTDRSTFQWLDTQPPGSVLYVGFGSQSSLEPEEFLALADGLVLSGQPFLWLVRPGFVKGYEWIEALPDGYLGPQGRIVKSAPQQEVLAHPAIGAFWTHGGWNGTMEAVCEGVPMIFSDFGLDQPLNARYMSEKLKIGVYLDDGWVPDRIAEAINLVMEEEKGAPYRQNAQALKDKLDRSIAPGGSSYESLERLQSYISSL

SEQ ID NO: 425 - protein, 457 aa, synthetic construct
MPNTNPTTVRRRRVVMFPVPFPGHLNPMLQLANVLYRRGFQITILYTNFNAPKTSLYPHFQFVPILDNDPQPEWLRNLPTTGPGVGARIPVINKHGAEALRAALEACIAATPSDEELACVITDALWYFAQPVAESLNLPLLVLQTGSLFNFHCLVCLPQFIELGLLDPNSTWADDAPVPGFPHLRVKDIRRAYSHIQESKPILMKMVEATKKSSGVIWNSAKELEESELETIQREIPAPSFLLPLPKHYRASSTSLLDTDPSTAAWLDTQPPGSVLYVGFGSQSSLSLEEFLALADGLVASGVPALWLVRPGFVKGKEWVDFLPDGYLGEKGRIVKSAPQQEVLAHPAIGAFWTHGGWNGTMEAVCEGVPMIFSDFGLDQPLNAAYMTRVKKIGVYLDRGFVKEDIVAAIKTVMTAPEGAEMRANAQALKEKLDRSIAPGGSSYESLERLQSYISSL

SEQ ID NO: 426 - protein, 457 aa, synthetic construct
MPNTNPTTVRRRTVVMFPVPFPGHLNPMLQLANVLYRRGFRIVILHTNFNAPKTSLYPHFQFIPILDNDPQPEWLRNLPTTGPGVGARIPVINKHGAPALKAALEACIAATPSDEELACVITDALWYFAQPVAESLGLKRLVLQTGSLFNFHCLVCLPRFIELGLLDPNSTWADDAPVPGFPHLRVKDIRRAYSHIQESKPILMKMVEETKKSSGVIWNSAKELEESELETIQREIPAPSFLLPLPKHYRASSTSLLDTDPSTAAWLDTQPPGSVLYVGFGSQSSLELEEALALADGLVRSGVPFLWLVRPGFVKGREWVDFLPDGYLGERGRIVKSAPQQEVLAHPAIGAFWTHGGWNGTMEAVCEGVPMIFSDFGLDQPLNAAYMTRVKKIGVYLDRGFRVDDIVAAVQTVMRAPEGAEMRANARELKEKLDRSIAPGGSSYESLERLQSYISSL

SEQ ID NO: 427 - protein, 457 aa, synthetic construct
MPNTNPTTVRRRRVVMFPVPFPGHLNPMLQLANVLYRRGFEITILYTNFNAPKTSLYPHFQFIPILDNDPQPDWLRNLPTTGPGVGARIPVINKHGAEQLKAALEACIAATPSDEELACLITDALWYFAQPVADSLGLKRLVLQTGSLFNFHCLVCLPKFLELGLLDPDSPWADDAPVPGFPHLTVKDIRRAYSHIQESKPILMKMVEETRKSSGVIWNSAKELEESELETIQREIPAPSFLLPLPKHYRASSTSLLDTDPSTAAWLDTQAPGSVLYVGFGSQSSLDKADFLALVDGLVASGQPFLWLVRPGFVKGEDWIEFLPEGYLGEKGRIVKSAPQQEVLAHPAIGAFWTHGGWNGTMEAVCEGVPMIFSDFGLDQPLNARYMVEVKKIGVYLDDGFVTAKIVDAINTVMKSEEGKEYRENAQKLKDKLDRSIAPGGSSYESLERLQSYISSL

SEQ ID NO: 428 - protein, 457 aa, synthetic construct
MPNTNPTTVRRRRVVMFPVPFPGHLNPMLQLANVLYRRGFEITILYTNFNAPKTSLYPHFQFIPVLDNDPQPEWLRNLPTTGPGVGARIPVINKHGAPELRAALEACIAATPSDEELACFITDALWYFAQPVAEELGLKRLVLQTGSLFNFHCLVCLPEFLALGLLDPDSTHADDAPVPGFPFLRVKDIRRAYSHIQESKPILMKMVEETRKSSGVIWNSAKELEESELETIQREIPAPSFLLPLPKHYRASSTSLLDTDPSTAAWLDTQPKGSVLYVGFGSQSSLSLEDFLALLDGLVKSGIPFLWLVRPGFVKGKEWIDFLPDGYLGEKGRIVKSAPQQEVLAHPAIGAFWTHGGWNGTMEAVCEGVPMIFSDFGLDQPLNARYMTEVKKIGVYLDDGFVVDKIVEAIKTVMESEEGKEMKANAQKLKDKLDRSIAPGGSSYESLERLQSYISSL

SEQ ID NO: 429 - protein, 457 aa, synthetic construct
MPNTNPTTVRRRTVVMFPVPFPGHLNPMLQLANVLYRRGFRIVILYTTFNAPKTSLYPHFQFIPILDNDPQPEWLRNLPTTGPGVGARIPVINKHGAEQLRAALEACIAATPSDEELACVITDALWYFAEPVAKSLGLKRLVLQTGSLFNFHCLVCLPRFLELGLLDPDSPWADDAPVPGFPFLTVKDIRRAYSHIQESKPILMKMVEETKKSSGVIWNSAKELEESELETIQREIPAPSFLLPLPKHYRASSTSLLDTDPSTAAWLDTQAPGSVLYVGFGSQSSLTKEEFLALLDGLKASGQPFLWLVRPGFVKGEDWISFIPEGYLGEKGRIVKSAPQQEVLAHPAIGAFWTHGGWNGTMEAVCEGVPMIFSDFGLDQPLNARYMVEVKKIGVYLDDGFVTDKIVAAIKTVMTSPEGAEMRANAQALKDKLDRSIAPGGSSYESLERLQSYISSL

SEQ ID NO: 430 - protein, 457 aa, synthetic construct
MPNTNPTTVRRRTVVMFPVPFPGHLNPMLQLANVLYRRGFRIVILHTRFNAPKTSLYPHFQFRPIIDNDPQPEYLRNLPTTGPGVGARIPVINKHGAEELRAALLECIAATPSDEELACVITDALWYFAYPVAKELGLPLLVLQTGSLFNFHCLVCLPEFIERGYLDPNSTHKDDEPVPGFPHLKVRDIRRAYSHIQESKPILMKMVEATKQSSGVIWNSAKELEESELETIQREIPAPSFLLPLPKHYRASSTSLLDTDPSTAAWLDTQPPESVLYVGFGSQSSMEEKDALAIAEGLVESGVPFLWLVRPGFVKGKEWVELLPDGYMGENGRIVKSAPQQEVLAHPAIGAFWTHGGWNGTMEAVCEGVPMIFSDFGLDQPLNARYMSDILKVGVYLDDGFVKDKIVEAIKTVMVDEKGKEYRANAKELKDKLDRSIAPGGSSYESLERLQSYISSL

SEQ ID NO: 431 - protein, 457 aa, synthetic construct
MPNTNPTTVRRRTVVMFPVPFPGHLNPMLQLANVLYRRGFRIIILHTNFNAPKTSLYPHFQFRPILDNDPQPEWLRNLPTTGPGVGARIPVINKHGAEELRAALLECIAATPSDEELACVITDALWYFAQPVADELGLKRLVLQTGSLFNFHCLVCLPEFLAAGILDPNSTLKDDTPVPGFPHLTVRDIRRAYSHIQESKPILMKMVEETKKSSGVIWNSAKELEESELETIQREIPAPSFLLPLPKHYRASSTSLLDTDPSTAAWLDTQPKESVLYVGFGSQSSLTEEEFLALAEGLVASGKPALWLVRPGFVKGKEWIELLPDGYLGEKGRIVKSAPQQEVLAHPAIGAFWTHGGWNGTMEAVCEGVPMIFSDFGLDQPLNARYMTDIKKIGVYLDDGFVKDKIVEAIKTVMEAEEGKEYKANAKALKEKLDRSIAPGGSSYESLERLQSYISSL

SEQ ID NO: 432 - protein, 457 aa, synthetic construct
MPNTNPTTVRRRTVVMFPVPFPGHLNPMLQLANVLYRRGFKIVILYTRFNAPKTSLYPHFQFRPVLDNDPQPEWLRNLPTTGPGVGARIPVINKHGAEALRAALLECIAATPSDEELACLITDALWYFAQPVADELGLKRLVLQTGSLFNFHCLVCLPKFLELGILDPNSTHKDDEPVPGFPHLRWKDIRRAYSHIQESKPILMKMVEETKRSSGVIWNSAKELEESELETIQREIPAPSFLLPLPKHYRASSTSLLDTDPSTAAWLDTQAPESVLYVGFGSQSSLTEEEFLAIAEGLVASGKPFLWLVRPGFVKGKDWIELLPDGYFGEKGRIVKSAPQQEVLAHPAIGAFWTHGGWNGTMEAVCEGVPMIFSDFGLDQPLNARYMTDIKKIGVYLDDGFVKDKIVEAIKTVMESEKGKEYRANAKELKDKLDRSIAPGGSSYESLERLQSYISSL

SEQ ID NO: 433 - protein, 457 aa, synthetic construct
MPNTNPTTVRRRTVVMFPVPFPGHLNPMLQLANVLYRRGFRIVILHTNFNAPKTSLYPHFQFRPILDNDPQPEWLRNLPTTGPGVGARIPVINKHGAEELRAALLECIAATPSDEELACVITDALWYFALPVAKELGLPLLVLQTGSLFNFHCLVCLPEFEELGILDPNSTHKDDEPVPGFPHLRWRDIRRAYSHIQESKPILMKMVEATKASSGVIWNSAKELEESELETIQREIPAPSFLLPLPKHYRASSTSLLDTDPSTAAWLDTQPPESVLYVGFGSQSSISEEDFLALAEGLVASGKPTLWLVRPGFVKGKEWIELLPDGYMGEKGRIVKSAPQQEVLAHPAIGAFWTHGGWNGTMEAVCEGVPMIFSDFGLDQPLNARYMSDIKKIGVYLDDGFVKDKIVEAIKTVMESEEGKEYRANAKELKAKLDRSIAPGGSSYESLERLQSYISSL

SEQ ID NO: 434 - protein, 457 aa, synthetic construct
MPNTNPTTVRRRTVVMFPVPFPGHLNPMLQLANVLYRRGFRIVILHTRFNAPKTSLYPHFQFIPILDNDPQPEWLRNLPTTGPGVGARIPVINKHGAPALRAALEACIAATPSDEELACVITDALWYFAQPVADELGLKRLVLQTGSLFNFHCLVCLPEFLEQGYLDPNSTHKDDEPVPGFPILKVKDIRRAYSHIQESKPILMKMVEETRRSSGVIWNSAKELEESELETIQREIPAPSFLLPLPKHYRASSTSLLDTDPSTAAWLDTQPPESVLYVGFGSQSSLSEEEALAIAEGLVRSGVPFLWLVRPGFVKGREWVELLPDGYLGERGRIVKSAPQQEVLAHPAIGAFWTHGGWNGTMEAVCEGVPMIFSDFGLDQPLNARYMSEKLKIGVYLDKGFVVDEIVKAIETVMRSEKGKEYRANARELKDKLDRSIAPGGSSYESLERLQSYISSL

SEQ ID NO: 435 - protein, 457 aa, synthetic construct
MPNTNPTTVRRRTVVMFPVPFPGHLNPMLQLANVLYRRGFRIIILHTRFNAPKTSLYPHFQFRPILDNDPQPEWLRNLPTTGPGVGARIPVINKHGAPELEAALKACIAATPSDEELACVITDALWYFAQPVADKLGLKRLVLQTGSLFNFHCLVCLPEFLELGILDPNSTHKDDEPVPGFPHLKWKDIRRAYSHIQESKPILMKMVEETKKSSGVIWNSAKELEESELETIQREIPAPSFLLPLPKHYRASSTSLLDTDPSTAAWLDTQPPRSVLYVGFGSQSSLTLEEALAIAEGLVRSGVPFLWLVRPGFVKGKEWIEFLPDGYFGEKGRIVKSAPQQEVLAHPAIGAFWTHGGWNGTMEAVCEGVPMIFSDFGLDQPLNARYMTDVKKIGVYLDNGFVVDDIVAAIHTVMRSEEGKEYRANAEELKDKLDRSIAPGGSSYESLERLQSYISSL

SEQ ID NO: 436 - protein, 457 aa, synthetic construct
MPNTNPTTVRRRHVVMFPVPFPGHLNPMLQLANVLYRRGFKITILYTNFNAPKTSLYPHFQFRPILDNDPQPEWLRNLPTTGPGVGARIPVINKHGAPELRAALKECIAATPSDEELACVITDALWYFAQPVAKELGLKRLVLQTGSLFNFHCLVCLPEFLAAGYLDPNSTLADDEPVPGFPFLTVKDIRRAYSHIQESKPILMKMVEETRKSSGVIWNSAKELEESELETIQREIPAPSFLLPLPKHYRASSTSLLDTDPSTAAWLDTQPPESVLYVGFGSQSSLTEEDFLALADGLVASGQPFLWLVRPGFVKGKEWIELLPDGYLGEKGRIVKSAPQQEVLAHPAIGAFWTHGGWNGTMEAVCEGVPMIFSDFGLDQPLNARYMTDKLKIGVYLDDGFVTDKIVEAINTVMKAEEGKEYKANAKALKDKLDRSIAPGGSSYESLERLQSYISSL

SEQ ID NO: 437 - protein, 457 aa, synthetic construct
MPNTNPTTVRRRTVVMFPVPFPGHLNPMLQLANVLYRRGFEIIILYTNFNAPKTSLYPHFQFIPVLDNDPQPEWLRNLPTTGPGVGARIPVINKHGAEQLKEALLKCIAATPSDEELACLITDALWYFATPVAKSLNIKRLVLQTGSLFNFHCLVCLPQFLADGYLDPNSPLKDDDPVPGFPFLRVRDIRRAYSHIQESKPILMKMVEETKASSGVIWNSAKELEESELETIQREIPAPSFLLPLPKHYRASSTSLLDTDPSTAQWLDTQPPGSVLYVGFGSQSSLELEDAKNIADGLVASGVPFLWLVRPGFVKGKDWIEFLPDGYLGEKGRIVKSAPQQEVLAHPAIGAFWTHGGWNGTMEAVCEGVPMIFSDFGLDQPLNARYMTEVKKIGVYLDDGFVKEKIVAAIHKVMKEEEGKEYRANAQALKDKLDRSIAPGGSSYESLERLQSYISSL

SEQ ID NO: 438 - protein, 457 aa, synthetic construct
MPNTNPTTVRRRRVVMFPVPFPGHLNPMLQLANVLYRRGFEITILYTNFNAPKTSLYPHFQFVQVLDNDPQPEWLRNLPTTGPGVGARIPVINKHGAPELEAALKACIAATPSDEELACLITDALWYFAQPVADSLNIKRLVLQTGSLFNFHCLVCLPKFLADGYLDPNSPLKDDDPVPGFPILRVKDIRRAYSHIQESKPILMKMVEETRKSSGVIWNSAKELEESELETIQREIPAPSFLLPLPKHYRASSTSLLDTDPSTAQWLDTQPAGSVLYVGFGSQSSLDEADFLALADGLVASGVPFLWLVRPGFVKGKDWIELLPDGYLGERGRIVKSAPQQEVLAHPAIGAFWTHGGWNGTMEAVCEGVPMIFSDFGLDQPLNARYMTEKKKVGVYLDDGFVTDKIVAAIHEVMKEEKGKEYRANAQKLKDKLDRSIAPGGSSYESLERLQSYISSL

SEQ ID NO: 439 - protein, 457 aa, synthetic construct
MPNTNPTTVRRRTVVMFPVPFPGHLNPMLQLANVLYRRGFRIVILHTNFNAPKTSLYPHFQFVRILDNDPQPEWLRNLPTTGPGVGARIPVINKHGAEALRKALEQCIADTPSDEEVACLITDALWYFAGPVAESLGLKRLVLQTGSLFNFHCLVCLPQFLEQGYLDPNSTLKDDDPVPGFPILRVKDIRRAYSHIQESKPILMKMVEETKRSSGVIWNSAKELEESELETIQREIPAPSFLLPLPKHYRASSTSLLDTDPSTAQWLDTQPDESVLYVGFGSQSSLEPEEFLNIAEGLVRSGVPFLWLVRPGFVKGYEWVELLPDGYLGEKGRIVKSAPQQEVLAHPAIGAFWTHGGWNGTMEAVCEGVPMIFSDFGLDQPLNARYMSEKLKIGVYLDEGFVVDRIVEAINTVMREEKGKEYRANAKALKDKLDRSIAPGGSSYESLERLQSYISSL

SEQ ID NO: 440 - protein, 457 aa, synthetic construct
MPNTNPTTVRRRTVVMFPVPFPGHLNPMLQLANVLYRRGFRIIILYTNFNAPKTSLYPHFQFRQVLDNDPQPEWLRNLPTTGPGVGARIPVINKHGAEQLRAALKECIAATPSDEEVACLITDALWYFAQPVADSLNLKRLVLQTGSLFNFHCLVCLPEFLEKGYLDPNSPLKDDDPVPGFPILKVRDIRRAYSHIQESKPILMKMVEETKKSSGVIWNSAKELEESELETIQREIPAPSFLLPLPKHYRASSTSLLDTDPSTAQWLDTQPAESVLYVGFGSQSSLEPEEFLALADGLVKSGIPFLWLVRPGFVKGKEWIELLPDGYLGEKGRIVKSAPQQEVLAHPAIGAFWTHGGWNGTMEAVCEGVPMIFSDFGLDQPLNARYMTEKLKIGVYLDNGFKTDDIVAAIHTVMKEEKGKEYRANAKKLKDKLDRSIAPGGSSYESLERLQSYISSL

SEQ ID NO: 441 - protein, 457 aa, synthetic construct
MPNTNPTTVRRRTVVMFPVPFPGHLNPMLQLANVLYRRGFKIIILHTNFNAPKTSLYPHFQFVQVLDNDPQPEWLRNLPTTGPGVGARIPVINKHGAEQLRKALLECIANTPSDEEVACLITDALWYFAQPVADSLNLKRLVLQTGSLFNFHCLVCLPEFLARGYLDPNSSLKDDDPVPGFPFLRVRDIRRAYSHIQESKPILMKMVEETKKSSGVIWNSAKELEESELETIQREIPAPSFLLPLPKHYRASSTSLLDTDPSTAQWLDTQPANSVLYVGFGSQSSLEPEEFLNIADGLVASGQPFLWLVRPGFVKGYEWIELLPDGYLGERGRIVKSAPQQEVLAHPAIGAFWTHGGWNGTMEAVCEGVPMIFSDFGLDQPLNARYMTEVKKIGVYLDDGFVKDKIVAAIKKVMEEEEGKEYRKNAQELKDKLDRSIAPGGSSYESLERLQSYISSL

SEQ ID NO: 442 - protein, 457 aa, synthetic construct
MPNTNPTTVRRRTVVMFPVPFPGHLNPMLQLANVLYRRGFKIVILHTNFNAPKTSLYPHFQFRQILDNDPQPEWLRNLPTTGPGVGARIPVINKHGAPQLEAALKQCIADTPSDEEVACLITDALWYFAQPVADSLNLKRLVLQTGSLFNFHCLVCLPEFLEQGYLDPNSPLKDDDPVPGFPFLRVKDIRRAYSHIQESKPILMKMVEETKKSSGVIWNSAKELEESELETIQREIPAPSFLLPLPKHYRASSTSLLDTDPSTAQWLDTQPPNSVLYVGFGSQSSLELEDALALADGLVASGVPFLWLVRPGFVKGKAWIEFLPDGYLGERGRIVKSAPQQEVLAHPAIGAFWTHGGWNGTMEAVCEGVPMIFSDFGLDQPLNARYMSEVKKIGVYLDDGFVTDKIVAAIHTVMKEEKGKEYRANAQALKDKLDRSIAPGGSSYESLERLQSYISSL

SEQ ID NO: 443 - protein, 457 aa, synthetic construct
MPNTNPTTVRRRRVIMFPVPFPGHLNPMLQLANVLYRRGFDITILYTNFNAPKTSLYPHFQFRQILDNDPQPEWLRNLPTTGPGVGARIPVINKHGAEQLREALLECIASTPSDEEVACLITDALWYFAMPVAKSLGLKRLVLQTGSLFNFHCLVCLPEFLAAGYLDPNSPLPDDAPVPGFPILKVADIRRAYSHIQESKPILMKMVEETRKSSGVIWNSAKELEESELETIQREIPAPSFLLPLPKHYRASSTSLLDTDPSTAQWLDTQPAGSVLYVGFGSQSSLEPEEFLNLADGLVKSGQPFLWLVRPGFVKGHEWIELLPDGYFGEKGRIVKSAPQQEVLAHPAIGAFWTHGGWNGTMEAVCEGVPMIFSDFGLDQPLNARYMSEKLKIGVYLDDGFVTDKIVAAINEVMKEEKGKEYRANAQELKDKLDRSIAPGGSSYESLERLQSYISSL

SEQ ID NO: 444 - protein, 457 aa, synthetic construct
MPNTNPTTVRRRTVVMFPVPFPGHLNPMLQLANVLYRRGFKIVILYTNFNAPKTSLYPHFQFRQILDNDPQPEWLRNLPTTGPGVGARIPVINKHGAEALREALLKCIAETPSDEELACLITDALWYFAQPVADSLGLKRLVLQTGSLFNFHCLVCLPDFLAQGYLDPNSPLKEDTPVPGFPILRVGDIRRAYSHIQESKPILMKMVEETRKSSGVIWNSAKELEESELETIQREIPAPSFLLPLPKHYRASSTSLLDTDPSTAQWLDTQPAGSVLYVGFGSQSSLEEEDFLNLADGLVESGQPFLWLVRPGFVKGKEWIELLPDGYLGEKGRIVKSAPQQEVLAHPAIGAFWTHGGWNGTMEAVCEGVPMIFSDFGLDQPLNARYMSEKLKIGVYLDNGFVKEDIVAAIKKVMEEEEGKEYRANAQKLKDKLDRSIAPGGSSYESLERLQSYISSL

SEQ ID NO: 445 - protein, 457 aa, synthetic construct
MPNTNTTVRRRRRIILFPVPFPGHLNPMLQLANVLYSKGFSITIFHTRFNLPKTSNYPHFTFKTLLDNDPQDEWLRNLPTHGPGVGARIPVINKHGAEQFEKLLKEYIEASESDEEVSCLITDALWYFAQDVAKKLGLRRLVLQTGSLFNFHCLVSLPQFEKQGYLDPSDTTNLDKPVEGFPWLKVKDIKRAYSHWQESKPILEKMVKQTKESSGVIWNSFKELEESELETVWREIPAPSFLIPLPKHYRASSTSLLDTDRSVFAWLDTQAPESVLYVGFGSQSEVEEADFLKIADGLVKSGIPFLWLVRPGFVKGQRWIEVLPEGYLGDRGRIVKSAPQQEVLAHPAIGAFWTHGGWNGTMESVCEGVPMIFSDFGLDQPLNARYMSEVLKVGVYLDNGWDPDEIAAAIRYVMRDEAGKPIRENAKALKQKADRSIMKGGSSYESLESLVSYISSL

SEQ ID NO: 446 - protein, 457 aa, synthetic construct
MPNTNTTVRRRRRIILFPVPFPGHLNPMLQLANVLYSKGFSITIFHTNFNKPKTSNYPHFTFIFVLDNDPQDEWLRNLPTHGPGVGARIPVINKHGAEQFEKILKEYIEASESDEKVSCLITDALWYFAEDVAKKLNLRRLVLQTGSLFNFHCLVSLPLFEKLGYLDPTDTTDLDKPAPGFPMLKVKDIKRAYSHWQESKPILEKMVKQTKASSGVIWNSFKELEESELETVWREIPAPSFLIPLPKHYRASSTSLLDTDRSVFAWLDTQAPESVLYVGFGSQSEVDEADFLKIAEGLVKSGIPFLWLVRPGFVKGKAWIEPLPEGFFGDKGRIVKSAPQQEVLAHPAIGAFWTHGGWNGTMESVCEGVPMIFSDFGLDQPLNARYMSDVLKIGVYLDNGWDPDAIAAAIRYVMRDEAGKPIRDNAKVLKQKADRSIMKGGSSYESLESLVSYISSL

SEQ ID NO: 447 - protein, 457 aa, synthetic construct
MPNTNTTVRRRRRIALFPVPFPGHLNPMLQLANVLYSKGFSITIFHTNFNKPKTSNYPHFTFVFILDNDPQDEWLRNLPTHGPGVGARIPVINKHGAEQFEKLLKEYIEASESDETVSCLITDALWYFAQEVADKLNLRRLVLQTGSLFNFHCLVSLPKFDELGYLDPDNKTNLDEPAEGFPMLKVKDIKRAYSHWQESKPILEKMVKQTKASSGVIWNSFKELEESELETVWREIPAPSFLIPLPKHYRASSTSLLDTDRSVFQWLDTQPPRSVLYVGFGSQSEVDVATFLAIADGLVRSGVPFLWLVRPGFVKGHTWIEPLPPGRLGPRGRIVKSAPQQEVLAHPAIGAFWTHGGWNGTMESVCEGVPMIFSDFGLDQPLNARYMSEVLKVGVYLRDGWDPDRIAAAIRYVMVDPAGAPIRENAQVLKQKADRSIMKGGSSYESLESLVSYISSL

SEQ ID NO: 448 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGAEQFKKLLKEYIEASESDETVSCLITDALWYFAQSVAEELNLRRLVLQTGSLFNFHCLVSLPRFDELGYLDPSNTTNLDAPAEGFPMLKVKDIKRAYSHWQESKPILEKMVKQTKASSGVIWNSFKELEESELETVWREIPAPSFLIPLPKHYRASSTSLLDTDRSVFDWLDTQAPESVLYVGFGSQSEVSEEDALKIADGLVKSGIPFLWLVRPGFIKGHEWIEPLPEGYLGDRGRIVKSAPQQEVLAHPAIGAFWTHGGWNGTMESVCEGVPMIFSDFGLDQPLNARYMSEVLKVGVYLDDGWDPDRIAAAIRLVMRDPEGKPIRENAKELKQKADRSIMKGGSSYESLESLVSYISSL

SEQ ID NO: 449 - protein, 457 aa, synthetic construct
MPNTNTTVRRRRRIILFPVPFPGHLNPMLQLANVLYSKGFSITIFHTNFNAPKTSNYPHFTFRFILDNDPQDEWLRNLPTHGPGVGARIPVINKHGAEQFEKELKEYIEASESDEEVSCLITDALWYFTQEVADKLNLPRLVLQTGSLFNFHCLVSLPKFEELGLLDPSDTTNLEEPVEGFPMLKRKDIKRAYSHWQESKPILEKMVKATKASSGVIWNSFKELEESELETVWREIPAPSFLIPLPKHYRASSTSLLDTDRSVFAWLDTQAPESVLYVGFGSQSEFSPEDFLKIVEGLVKSGIPTLWLVRPGFVKGSEWIEVIPEGYLGDKGRIVKSAPQQEVLAHPAIGAFWTHGGWNGTMESVCEGVPMIFSDFGLDQPLNARYMSEVLKIGVYLDNGFDPDEIAAAIRYVMRDEAGKPIRDNAKELKEKADRSIMKGGSSYESLESLVSYISSL

SEQ ID NO: 450 - protein, 457 aa, synthetic construct
MPNTNTTVRRRRRIILFPVPFPGHLNPMLQLANVLYSKGFSITIFHTNFNRPKTSNYPHFTFRFILDNDPQDEWLRNLPTHGPGVGARIPVINKHGAEQFEKELKAYIEASESDETVSCLITDALWYFAQDVAKRLNLRRLVLQTGSLFNFHCLVSLPQFEKLGYLDPSNNTNLDKPAEGFPMLKVKDIKRAYSHWQESKPILEKMVKQTKESSGVIWNSFKELEESELETVWREIPAPSFLIPLPKHYRASSTSLLDTDRSVFAWLDTQAPESVLYVGFGSQSEVSVAEALAIADGLVRSGIPFLWLVRPGFIKGEEWIEALPEGRLGDKGRIVKSAPQQEVLAHGAIGAFWTHGGWNGTMESVCEGVPMIFSDFGLDQPLNARYMSEVLKVGVYLDDGWDPDAIAAAIRYVMRDEAGKPIRENAKKLKQKADRSIMKGGSSYESLESLVSYISSL

SEQ ID NO: 451 - protein, 457 aa, synthetic construct
MPNTNTTVRRRRRIILFPVPFPGHLNPMLQLANVLYSKGFSITIIHTNFNRPKTSNYPHFTFKTLLDNDPQDEWLRNLPTHGPGVGARIPVINKHGAEKFEKLLKELIEASESDEEVSCLITDALWYFAQDVADKLGLRRLVLQTGSLFNFHCLVSLPQFEEQGYLDPDNQTDLDKPAPGFPMLKVKDIKRAYSHWQESKPILEKMVKQTKASSGVIWNSFKELEESELETVWREIPAPSFLIPLPKHYRASSTSLLDTDRSVFQWLDTQPPESVLYVGFGSQSEVEVADFLKIAEGLVKSGLPFLWLVRPGFVKGEKWIESLPEGFLGPKGRIVKSAPQQEVLAHPAIGAFWTHGGWNGTMESVCEGVPMIFSDFGLDQPLNARYMSEVLKVGVYLDRGWDPDEIAAAIRLVMRDEAGAPIRENAKVLKQKADRSIMKGGSSYESLESLVSYISSL

SEQ ID NO: 452 - protein, 457 aa, synthetic construct
MPNTNTTVRRRRRIILFPVPFPGHLNPMLQLANVLYSKGFSITIFHTTFNKPKTSNYPHFTFRFFLDNDPQDEWLRNLPTHGPGVGARIPVINKHGAEKFEKMLKEYIEASESDEEVSCLITDALWYFAQDVAKKLNLRRLVLQTGSLFNFHCLVSLPKFDELGYLDPDDKTDLDKPVEGFPELKVKDIKRAYSHWQESKPILEKMVKQTKESSGVIWNSFKELEESELETVWREIPAPSFLIPLPKHYRASSTSLLDTDRSVFAWLDTQPPESVLYVGFGSQSEVDEATFLAIAEGLVRSGLPFLWLVRPGFVKGHRWIEPLPEGYLGPRGRIVKSAPQQEVLAHPAIGAFWTHGGWNGTMESVCEGVPMIFSDFGLDQPLNARYMSEVLKVGVYLENGWDPAAIAAAIRLVMRDPAGAPIRENAKVLKQKADRSIMKGGSSYESLESLVSYISSL

SEQ ID NO: 453 - protein, 457 aa, synthetic construct
MPNTNTTVRRRRRIILFPVPFPGHLNPMLQLANVLYSKGFSITIFHTNFNKPKTSNYPHFTFVFLLDNDPQDEWLRNLPTHGPGVGARIPVINKHGAEQFYKTLKAYIEASESDEEVSCLITDALWYFAQSVADRLGLRRLVLQTGSLFNFHCLVSLPKFDELGYLDPDNKENLDEPVEGFPELKVKDIKRAYSHWQESKPILEKMVKQTKESSGVIWNSFKELEESELETVWREIPAPSFLIPLPKHYRASSTSLLDTDRSVFQWLDQQAPESVLYVGFGSQSEVDPETFLRIAEGLVKSGLPFLWLVRPGFVKGETWIEVLPEGYLGPRGRIVKSAPQQEVLAHPAIGAFWTHGGWNGTMESVCEGVPMIFSDFGLDQPLNARYMSEVLKVGVYLENGWDPEEIARAIRLVMRDPEGAPIRENARVLKQKADRSIMKGGSSYESLESLVSYISSL

SEQ ID NO: 454 - protein, 457 aa, synthetic construct
MPNTNTTVRRRRRIILFPVPFPGHLNPMLQLANVLYSKGFSITIFHTNFNKPKTSNYPHFTFRFILDNDPQDEWLRNLPTHGPGVGARIPVINKHGAEQFEKELKEYIEASESDETVSCLITDALWYFAQDVAKKLNLPRLVLQTGSLFNFHCLVSLPKFEELGYLNPDDQTNLEEPAEGFPMLKVRDIKRAYSHWQESKPILEKMVKATKESSGVIWNSFKELEESELETVWREIPAPSFLIPLPKHYRASSTSLLDTDRSVFDWLDTQAPESVLYVGFGSQSEVSPADALAIAEGLVKSGVPFLWLVRPGFIKGHTWIEVLPEGFLGDRGRIVKSAPQQEVLAHPAIGAFWTHGGWNGTMESVCEGVPMIFSDFGLDQPLNARYMSEVLKVGVYLRDGFDPDAIAAAIRLVMRDEAGKPIRENAKKLKEKADRSIMKGGSSYESLESLVSYISSL

SEQ ID NO: 455 - protein, 457 aa, synthetic construct
MPNTNTTVRRRRRIILFPVPFPGHLNPMLQLANVLYSKGFSITIFHTNFNKPKTSNYPHFTFIEILDNDPQDEWLRNLPTHGPGVGARIPVINKHGAEKFEKILKEYIEASESDEKVSCLITDALWYFTEDVAKKLNLRRLVLQTGSLFNFHCLVSLPKFEELGYLDPDNTENLEEPAEGFPMLKVKDIKRAYSHWQESKPILEKMVKQTKASSGVIWNSFKELEESELETVWREIPAPSFLIPLPKHYRASSTSLLDTDRSVFDWLDTQAPESVLYVGFGSQSEVTEEEFLKIVDGLVKSGIPFLWLVRPGFVKGKEWIEPIPEGYLGDKGRIVKSAPQQEVLAHPAIGAFWTHGGWNGTMESVCEGVPMIFSDFGLDQPLNARYMSEVKKVGVYLDNGWDPDEIAAAIRYVMKDEAGKPIRDNAKELKQKADRSIMKGGSSYESLESLVSYISSL

SEQ ID NO: 456 - protein, 457 aa, synthetic construct
MPNTNTTVRRRRRIILFPVPFPGHLNPMLQLANVLYSKGFSITIIHTNFNKPKTSNYPHFTFLTLLDNDPQDEWLRNLPTHGPGVGARIPVINKHGAEQFEKLLKEYIEASESDEEVSCLITDALWYFAQSVADKLNLRRLVLQTGSLFNFHCLVSLPKFEELGYLDPENKSNLEEPAEGFPELKVKDIKRAYSHWQESKPILEKMVKQTKESSGVIWNSFKELEESELETVWREIPAPSFLIPLPKHYRASSTSLLDTDRSVFAWLDTQAPESVLYVGFGSQSEVEPEEFLAIAEGLVKSGLPFLWLVRPGFVKGYRWIEPLPEGYLGPRGRIVKSAPQQEVLAHPAIGAFWTHGGWNGTMESVCEGVPMIFSDFGLDQPLNARYMSEVLKVGVYLDKGWDPDEIAAAIRYVMRDEAGKPIRENAKALKQKADRSIMKGGSSYESLESLVSYISSL

SEQ ID NO: 457 - protein, 457 aa, synthetic construct
MPNTNTTVRRRRRIALFPVPFPGHLNPMLQLANVLYSKGFSITIFHTNFNKPKTSNYPHFTFIFILDNDPQDEWLRNLPTHGPGVGARIPVINKHGAEPFKKTLKAYIEASESDETLSCLITDALWYFAQSVADELNLRRLVLQTGSLFNFHCLVSLPQFDQLGYLDPSNKTNLEQPAEGFPMLRVKDIKRAYSHWQESKPILEKMVKQTKASSGVIWNSFKELEESELETVWREIPAPSFLIPLPKHYRASSTSLLDTDRSVFDWLDTQAPRSVLYVGFGSQSEVDPETFLAIADGLVKSGIPFLWLVRPGFVKGYEWIEPLPEGYLGDKGRIVKSAPQQEVLAHPAIGAFWTHGGWNGTMESVCEGVPMIFSDFGLDQPLNARYMSEVLKVGVYLRDGWDPDAIAAAIRLVMVDEAGKPIRENAEVLKQKADRSIMKGGSSYESLESLVSYISSL

SEQ ID NO: 458 - protein, 457 aa, synthetic construct
MPNTNTTVRRRRRIILFPVPFPGHLNPMLQLANVLYSKGFSITIIHTTFNKPKTSNYPHFTFVFLLDNDPQDEWLRNLPTHGPGVGARIPVINKHGKEQFKKLLSELIEASESDEEVSCLITDALWYFAQEVADELGLRRLVLQTGSLFNFHCLVSLPKFDELGYLDPEDQSDLDKPVEGFPELKVKDIKRAYSHWQESKPILEKMVKQTKESSGVIWNSFKELEESELETVWREIPAPSFLIPLPKHYRASSTSLLDTDRSVFAWLDTQAPESVLYVGFGSQSEVDRETFLKIAEGLVKSGLPFLWLVRPGFVKGERWIESLPEGYLGPRGRIVKSAPQQEVLAHPAIGAFWTHGGWNGTMESVCEGVPMIFSDFGLDQPLNARYMSEVLKVGVYLDEGWDPDRIAAAIRLVMVDEAGRPIRENARVLKQKADRSIMKGGSSYESLESLVSYISSL

SEQ ID NO: 459 - protein, 457 aa, synthetic construct
MPNTNTTVRRRRRIILFPVPFPGHLNPMLQLANVLYSKGFSITIFHTNFNKPKTSNYPHFTFVFILDNDPQDEWLRNLPTHGPGVGARIPVINKHGAEQFKKELKAYIEASESDEEVSCLITDALWYFAQDVAKELNLRRLVLQTGSLFNFHCLVSLPQFEKLGYLDPSNNTDLDKPAEGFPMLKVKDIKRAYSHWQESKPILEKMVKQTKESSGVIWNSFKELEESELETVWREIPAPSFLIPLPKHYRASSTSLLDTDRSVFAWLDTQAPRSVLYVGFGSQSEVDEADFLKIAEGLVKSGVPFLWLVRPGFVKGHTWIEVLPEGYLGPKGRIVKSAPQQEVLAHPAIGAFWTHGGWNGTMESVCEGVPMIFSDFGLDQPLNARYMSEVLKVGVYLDNGWDPDEIAAAIRYVMRDPAGAPIRENAEVLKQKADRSIMKGGSSYESLESLVSYISSL

SEQ ID NO: 460 - protein, 457 aa, synthetic construct
MPNTNTTVRRRRRIILFPVPFPGHLNPMLQLANVLYSKGFSITIFHTNFNKPKTSNYPHFTFIEILDNDPQDEWLRNLPTHGPGVGARIPVINKHGAEPFEKVLKEYIEASESDEEVSCLITDALWYFAQDVAKKLNLRRLVLQTGSLFNFHCLVSLPKFEQLGYLDPSNQKDLDKPAEGFPMLKVKDIKRAYSHWQESKPILEKMVKQTKESSGVIWNSFKELEESELETVWREIPAPSFLIPLPKHYRASSTSLLDTDRSVFEWLDTQAPESVLYVGFGSQSEVDPATFLAIAEGLVKSGISFLWLVRPGFVKGSEWIEPLPEGYLGDRGRIVKSAPQQEVLAHPAIGAFWTHGGWNGTMESVCEGVPMIFSDFGLDQPLNARYMSEVLKVGVYLDDGWDPDRIAAAIRLVMRDEAGAPIRENAKVLKQKADRSIMKGGSSYESLESLVSYISSL

SEQ ID NO: 461 - protein, 457 aa, synthetic construct
MPNTNTTVRRRRRIILFPVPFPGHLNPMLQLANVLYSKGFSITIFHTTFNKPKTSNYPHFTFIFFLDNDPQDEWLRNLPTHGPGVGARIPVINKHGKEKFLKLLKEYIEASESDEKISCLITDALWYFAQDVADELGLRRLVLQTGSLFNFHCLVSLPKFDELGYLDPNDKTNLEKPAEGFPWLKVKDIKRAYSHWQESKPILEKMVKQTKASSGVIWNSFKELEESELETVWREIPAPSFLIPLPKHYRASSTSLLDTDRSVFAWLDTQAPGSVLYVGFGSQSEVDVETFLKIVEGLVKSGIPFLWLVRPGFVKGKEWIEPIPEGYLGDRGRIVKSAPQQEVLAHPAIGAFWTHGGWNGTMESVCEGVPMIFSDFGLDQPLNARYMSDVLKVGVYLDNGWDPDEIAAAIRYVMRDEAGAPIRENAKVLKQKADRSIMKGGSSYESLESLVSYISSL

SEQ ID NO: 462 - protein, 457 aa, synthetic construct
MPNTNTTVRRRRRIVLFPVPFPGHLNPMLQLANVLYSKGFSITIFHTTFNRPKTSNYPHFTFVFLLDNDPQDEWLRNLPTHGPGVGARIPVINKHGAEQFEKLLKEYIEASESDEEVSCLITDALWYFAQDVADRLGLRRLVLQTGSLFNFHCLVSLPQFEEQGLLNPEDQSNLDQPVPGFPELKAKDIKRAYSHWQESKPILEKMVKQTKASSGVIWNSFKELEESELETVWREIPAPSFLIPLPKHYRASSTSLLDTDRSVFAWLDTQAPESVLYVGFGSQSEVEPETFLAIAEGLVKSGVSFLWLVRPGFVKGSRWIEVLPEGYLGPRGRIVKSAPQQEVLAHGAIGAFWTHGGWNGTMESVCEGVPMIFSDFGLDQPLNARYMSEVLKVGVYLDNGWDPDEIAAAIRYVMRDPAGAYIRENAKKLKQKADRSIMKGGSSYESLESLVSYISSL

SEQ ID NO: 463 - protein, 457 aa, synthetic construct
MPNTNTTVRRRRRIILFPVPFPGHLNPMLQLANVLYSKGFSITIFHTNFNKPKTSNYPHFTFITILDNDPQDEWLRNLPTHGPGVGARIPVINKHGAEQFKKVLKSYIEASESDETVSCLITDALWYFTQDVAKELNLRRLVLQTGSLFNFHCLVSLPRFEELGYLDPDNQTDLEKPAEGFPMLKVKDIKRAYSHWQESKPILEKMVKQTKESSGVIWNSFKELEESELETVWREIPAPSFLIPLPKHYRASSTSLLDTDRSVFAWLDTQAPRSVLYVGFGSQSEVSVEEALAIAEGLVKSGVPFLWLVRPGFVKGATWIEPLPEGFLGDRGRIVKSAPQQEVLAHPAIGAFWTHGGWNGTMESVCEGVPMIFSDFGLDQPLNARYMSEVLKVGVYLDEGWDPDRIAAAIRLVMRDEAGKPIRENALELKQKADRSIMKGGSSYESLESLVSYISSL

SEQ ID NO: 464 - protein, 457 aa, synthetic construct
MPNTNTTVRRRRRIILFPVPFPGHLNPMLQLANVLYSKGFSITIFHTTFNAPKTSNYPHFTFRFLLDNDPQDEWLRNLPTHGPGVGARIPVINKHGAEQFEKMLKEYIEASESDEEVSCLITDALWYFAQEVADKLGLRRLVLQTGSLFNFHCLVSLPRFEQQGYLDPSNQSNLDQPVEGFPELLVKDIKRAYSHWQESKPILEKMVKQTKASSGVIWNSFKELEESELETVWREIPAPSFLIPLPKHYRASSTSLLDTDRSVFAWLDTQAPESVLYVGFGSQSEVEEADFLAIAEGLVKSGISFLWLVRPGFVKGSRWIEPLPEGYLGPRGRIVKSAPQQEVLAHGAIGAFWTHGGWNGTMESVCEGVPMIFSDFGLDQPLNARYMSDVLKVGVYLRNGWDPDEIAAAIRYVMVDPEGEPIRKNAKELKQKADRSIMKGGSSYESLESLVSYISSL

SEQ ID NO: 465 - protein, 457 aa, synthetic construct
MPNTNTTVRRRRRIILFPVPFPGHLNPMLQLANVLYSKGFSITIFHTNFNAPKTSNYPHFTFRFILDNDPQDEWLRNLPTHGPGVGARIPVINKHGAEQFEKELKEFIEASESDEEVSCLITDALWYFAQEVADRLNLRRLVLQTGSLFNFHCLVSLPQFEKLGYLDPSNQTNLDKPAEGFPMLKVKDIKRAYSHWQESKPILEKMVKQTKASSGVIWNSFKELEESELETVWREIPAPSFLIPLPKHYRASSTSLLDTDRSVFAWLDTQPPRSVLYVGFGSQSEVEVEEALKIAEGLVKSGIPFLWLVRPGFVKGEEWIEALPEGFLGDRGRIVKSAPQQEVLAHPAIGAFWTHGGWNGTMESVCEGVPMIFSDFGLDQPLNARYMSEVLKVGVYLDNGWDPDEIARAIRLVMVDPEGAPIRENAKALKQKADRSIMKGGSSYESLESLVSYISSL

SEQ ID NO: 466 - protein, 457 aa, synthetic construct
MPNTNTTVRRRRRIILFPVPFPGHLNPMLQLANVLYSKGFSITIFHTNFNKPKTSNYPHFTFVTILDNDPQDEWLRNLPTHGPGVGARIPVINKHGAEQFEKLLKEYIEASESDEEVSCLITDALWYFAQDVADKLNLRRLVLQTGSLFNFHCLVSLPKFEELGLLDPADTTGLEEPVPGFPWLKRKDIKRAYSHWQESKPILEKMVKQTKASSGVIWNSFKELEESELETVWREIPAPSFLIPLPKHYRASSTSLLDTDRSVFAWLDTQAPGSVLYVGFGSQSEVDEATFLKIVDGLVKSGIPFLWLVRPGFVKGKEWIEPIPEGYLGDKGRIVKSAPQQEVLAHPAIGAFWTHGGWNGTMESVCEGVPMIFSDFGLDQPLNARYMSEVLKVGVYLDDGWDPDAIAAAIRYVMRDPAGAPIRENAKELKQKADRSIMKGGSSYESLESLVSYISSL

SEQ ID NO: 467 - protein, 457 aa, synthetic construct
MPNTNTTVRRRRRIILFPVPFPGHLNPMLQLANVLYSKGFSITILHTRFNKPKTSNYPHFTFVEILDNEPQDEWLRNLPTHGPGVGARIPVINKHGAEELRKVLEELIAASESDERVSCLITDALWYFAQDVAKSLNLRRLVLQTGSLFNFHCLVSLPHFEKLGYLDPNNKTRLDEPAEGFPMLKVKDIKRAYSHWQESKPILEKMVKQTKESSGVIWNSFKELEESELETVWREIPAPSFLIPLPKHYRASSTSLLDTDRSVFDWLDTQPPRSVLYVGFGSQSEVEPADFLAIVDGLVASGQPFLWLVRPGFVKGYEWIEPLPEGRLGERGRIVKSAPQQEVLAHPAIGAFWTHGGWNGTMESVCEGVPMIFSDFGLDQPLNARYMSEVLKVGVYLDEGWDPARIAAAIRLVMVDPAGAPIRENARVLKQKADRSIMKGGSSYESLESLVSYISSL

SEQ ID NO: 468 - protein, 457 aa, synthetic construct
MPNTNTTVRRTRRIILFPVPFPGHLNPMLQLANVLYSKGFSITILHTNFNKPKTSNYPHFTFRSILDNDPQDEWLRNLPTHGPGVGARIPVINKHGAAEFRAELERIIAASESDETVSCLITDALWYFAQDVADSLGLRRLVLQTGSLFNFHCLVSLPKFEELGYLDPNNKEDLDKPAEGFPMLKVKDIKRAYSHWQESKPILEKMVKQTKASSGVIWNSFKELEESELETVWREIPAPSFLIPLPKHYRASSTSLLDTDRSVFAWLDRQPPESVLYVGFGSQSEVDPEDFLKIVDGLIKSGLPFLWLVRPGFVKGYEWIEVLPEGRLGERGRIVKSAPQQEVLAHPAIGAFWTHGGWNGTMESVCEGVPMIFSDFGLDQPLNARYMSEVLKVGVYLDRGWDPDEIAAAIRLVMVDPAGKPIRENARVLKQKADRSIMKGGSSYESLESLVSYISSL

SEQ ID NO: 469 - protein, 457 aa, synthetic construct
MPNTNTTVRRRRRIILFPVPFPGHLNPMLQLANVLYSKGFSITILHTNFNKPKTSNYPHFTFVEILDNEPQDEWLRNLPTHGPGVGARIPVINKHGAEEFEKTLKELIAASESDETVSCLITDALWYFAQDVADSLNLRRLVLQTGSLFNFHCLVSLPLFEKLGYLDPNDTSNLDEPAEGFPMLKVKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVTPEEFLAIVDGLVASEIPFLWLVRPGFVKGYDWIEPLPEGYLGERGRIVKSAPQQEVLAHPAIGAFWTHGGWNGTMESVCEGVPMIFSDFGLDQPLNARYMSEKLKIGVYLDDGWDPEKIAAAIRYVMRDPAGQPIRDNAKVLKQKADRSIMKGGSSYESLESLVSYISSL

SEQ ID NO: 470 - protein, 457 aa, synthetic construct
MPNTNTTVRRRRRIILFPVPFPGHLNPMLQLANVLYSKGFSITILHTRFNAPKTSNYPHFTFVTILDNEPQDEWLRNLPTHGPGVGARIPVINKHGADELRKKLEELIAASESDERVACLITDALWYFAQDVAKSLNLPRLVLQTGSLFNFHCLVSLPQFEKLGYLDPNNNTNLEEPAEGFPMLKVKDIKRAYSHWQESKPILEKMVKATKESSGVIWNSFKELEESELETVWREIPAPSFLIPLPKHYRASSTSLLDTDRSVFAWLDTQPPESVLYVGFGSQSEVDPKEFLEIADGLVASGLPFLWLVRPGFVKGEEWIEVLPEGYLGERGRIVKSAPQQEVLAHPAIGAFWTHGGWNGTMESVCEGVPMIFSDFGLDQPLNAAYMSRVLKVGVYLDNGFDREAIAAAIRLVMVDPAGEPIRQNARELKEKADRSIMKGGSSYESLESLVSYISSL

SEQ ID NO: 471 - protein, 457 aa, synthetic construct
MPNTNTTVRRRRTIILFPVPFPGHLNPMLQLANVLYSKGFSIIILHTRFNAPKTSNYPHFTFREILDREPQDEWLRNLPTHGPGVGARIPVINKHGAEEFRKELEEIIAASESDETVSCLITDALWYFTQDVADSLNLRRLVLQTGSLFNFHCLVSLPKFEELGLLDPNNKENLEEPVEGFPMLKRKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVTPEEFLEIVDGLVKSKIPFLWLVRPGFVKGYEWIEPLPEGYLGERGRIVKSAPQQEVLAHPAIGAFWTHGGWNGTMESVCEGVPMIFSDFGLDQPLNARYMSDVLKIGVYLDNGWNPEEIAAAIRYVMEDPEGEPIRQNAKELKQKADRSIMKGGSSYESLESLVSYISSL

SEQ ID NO: 472 - protein, 457 aa, synthetic construct
MPNTNTTVRRRRRIILFPVPFPGHLNPMLQLANVLYSKGFSITILHTRFNAPKTSNYPHFTFRTILDNDPQDEWLRNLPTHGPGVGARIPVINKHGAEELRKTLEELIAASESDEEVSCLITDALWYFAQDVADSLGLRRLVLQTGSLFNFHCLVSLPKFEELGLLDPNDQTNLEEPVPGFPWLKAKDIKRAYSHWQESKPILEKMVKQTKESSGVIWNSFKELEESELETVWREIPAPSFLIPLPKHYRASSTSLLDTDRSVFAWLDTQPPESVLYVGFGSQSEVEPEDFLAIVDGLVRSGLPFLWLVRPGFVKGEEWIEVLPEGRLGERGRIVKSAPQQEVLAHPAIGAFWTHGGWNGTMESVCEGVPMIFSDFGLDQPLNARYMSEVLKVGVYLDDGWDPEAIAAAIRLVMVDPEGEPIRQNAKELKQKADRSIMKGGSSYESLESLVSYISSL

SEQ ID NO: 473 - protein, 457 aa, synthetic construct
MPNTNTTVRRRRTIILFPVPFPGHLNPMLQLANVLYSKGFNIIILHTAFNAPKTSNYPHFTFRTILDRDPQDEWLRNLPTHGPGVGARIPVINKHGAEEFRKVLEELIAASESDETVSCLITDALWYFALDVAKSLNLRRLVLQTGSLFNFHCLVSLPKFEELGLLDPNDTTGLEEPVPGFPYLRRKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVSPEDFLKIVDGLVASGLPFLWLVRPGFVKGYEWIEPLPEGYLGEKGRIVKSAPQQEVLAHPAIGAFWTHGGWNGTMESVCEGVPMIFSDFGLDQPLNARYMSEVKKVGVYLDNGWDPEEIAAAIRYVMKDEAGKPIRDNAKELKQKADRSIMKGGSSYESLESLVSYISSL

SEQ ID NO: 474 - protein, 457 aa, synthetic construct
MPNTNTTVRRRRRIILFPVPFPGHLNPMLQLANVLYSKGFSITILHTNFNKPKTSNYPHFTFVNILDNEPQDEWLRNLPTHGPGVGARIPVINKHGAAELKKTLEELIAASESDETVSCLITDALWYFAQDVAKSLNLRRLVLQTGSLFNFHCLVSLPKFEELGYLDPNNNTNLEEPAEGFPMLKVKDIKRAYSHWQESKPILEKMVKQTKESSGVIWNSFKELEESELETVWREIPAPSFLIPLPKHYRASSTSLLDTDRSVFAWLDTQPPRSVLYVGFGSQSEVDREDFLKIVEGLVKSGLPFLWLVRPGFVKGEEWIEVLPEGYLGERGRIVKSAPQQEVLAHPAIGAFWTHGGWNGTMESVCEGVPMIFSDFGLDQPLNARYMSEVLKVGVYLDNGWDPEEIAAAARLVMVDPAGEPIRRNAEVLKQKADRSIMKGGSSYESLESLVSYISSL

SEQ ID NO: 475 - protein, 457 aa, synthetic construct
MPNTNTTVRRRDRIILFPVPFPGHLNPMLQLANVLYSKGFSITILHTNFNRPKTSNYPHFTFVTVLDNDPQDEWLRNLPTHGPGVGARIPVINKHGAERLREILESLIAASESDERIRCLITDALWYFAQDVAKSLNLRRLVLQTGSLFNFHCLVSLPQFEKLGYLDPSNKTRLDEPAEGFPLLKVKDIKRAYSHWQESKPILEKMVKQTKESSGVIWNSFKELEESELETVWREIPAPSFLIPLPKHYRASSTSLLDTDRSVFDWLDQQPPESVLYVGFGSQSEVTPEEALAIAEGLVRSGVPFLWLVRPGFVKGQAWIEVLPPGFLGEKGRIVKSAPQQEVLAHGAIGAFWTHGGWNGTMESVCEGVPMIFSDFGLDQPLNAAYMSRELKIGVYLDNGFNPEEIARAIRLVMEDEEGKPIRENARELKEKADRSIMKGGSSYESLESLVSYISSL

SEQ ID NO: 476 - protein, 457 aa, synthetic construct
MPNTNTTVRRRRRIILFPVPFPGHLNPMLQLANVLYSKGFSITILHTNFNKPKTSNYPHFTFVNVLDNDPQDEWLRNLPTHGPGVGARIPVINKHGAEKLKKVLEEIIAASESDETVSCLITDALWYFAQDVAKSLNLRRLVLQTGSLFNFHCLVSLPKFEKLGYLDPSNKTNLDKPAEGFPLLKVKDIKRAYSHWQESKPILEKMVKQTKESSGVIWNSFKELEESELETVWREIPAPSFLIPLPKHYRASSTSLLDTDRSVFDWLAQQPPRSVLYVGFGSQSEVTPEEFLTIAEGLVRSGVPFLWLVRPGFVKGQAWIEVLPPGFLGEKGRIVKSAPQQEVLAHPAIGAFWTHGGWNGTMESVCEGVPMIFSDFGLDQPLNARYMSEKLKIGVYLDEGWDPERIAAAIRLVMQDPAGEPIRQNAEVLKQKADRSIMKGGSSYESLESLVSYISSL

SEQ ID NO: 477 - protein, 457 aa, synthetic construct
MPNTNTTVRRTRRIVLFPVPFPGHLNPMLQLANVLYSKGFSITILHTNFNKPKTSNYPHFTFVTVLDNDPQDEWLRNLPTHGPGVGARIPVINKHGAEKLRQTLEQLLAASESDETISCLITDALWYFAQPVADSLGLRRLVLQTGSLFNFHCLVSLPRFEELGYLDPSDQSDLEKPAEGFPLLKVKDIKRAYSHWQESKPILEKMVKQTKESSGVIWNSFKELEESELETVWREIPAPSFLIPLPKHYRASSTSLLDTDRSVFDWLDRQPPESVLYVGFGSQSEVTPEEFLTIAEGLVRSGIPFLWLVRPGFVKGQSWIEVLPDGFLGERGRIVKSAPQQEVLAHGAIGAFWTHGGWNGTMESVCEGVPMIFSDFGLDQPLNARYMSEKLRIGVYLDDGWDPERIAAAIRLVMQDPAGAPIRENARVLKQKADRSIMKGGSSYESLESLVSYISSL

SEQ ID NO: 478 - protein, 457 aa, synthetic construct
MPNTNTTVRRRRRIILFPVPFPGHLNPMLQLANVLYSKGFSITILHTRFNKPKTSNYPHFTFVEVLDNDPQDEWLRNLPTHGPGVGARIPVINKHGAEPLRQVLEQILAASESDETVSCLITDALWYFAQEVADSLNLRRLVLQTGSLFNFHCLVSLPQFEQLGYLDPSNQTRLDEPAEGFPLLKVKDIKRAYSHWQESKPILEKMVKQTKASSGVIWNSFKELEESELETVWREIPAPSFLIPLPKHYRASSTSLLDTDRSVFDWLATQPPESVLYVGFGSQSEVTPEEFLTIAEGLVRSGIPFLWLVRPGFVKGQRWIEVLPEGFLGERGRIVKSAPQQEVLAHPAIGAFWTHGGWNGTMESVCEGVPMIFSDFGLDQPLNARYMSEKLKIGVYLDRGWDPERIAAAIRLVMVDEKGKPIRENARVLKQKADRSIMKGGSSYESLESLVSYISSL

SEQ ID NO: 479 - protein, 457 aa, synthetic construct
MPNTNTTVRRRRRIILFPVPFPGHLNPMLQLANVLYSKGFSITILHTNFNLPKTSNYPHFTFVTVLDNDPQDEWLRNLPTHGPGVGARIPVINKHGAERLREVLEELMAASESDEEISCLITDALWYFAQEVADSLNLRRLVLQTGSLFNFHCLVSLPEFEKLGLLDPSNQTNLDEPAPGFPWLKNKDIKRAYSHWQESKPILEKMVKQTKASSGVIWNSFKELEESELETVWREIPAPSFLIPLPKHYRASSTSLLDTDRSVFDWLAQQPPRSVLYVGFGSQSEVTPEEALTIAEGLVRSGIPFLWLVRPGFVKGQAWIEVLPPGFLGERGRIVKSAPQQEVLAHPAIGAFWTHGGWNGTMESVCEGVPMIFSDFGLDQPLNARYMSEVLKIGVYLDEGWDPERIAAAIRLVMRDESGKPIRENALALKQKADRSIMKGGSSYESLESLVSYISSL

SEQ ID NO: 480 - protein, 457 aa, synthetic construct
MPNTNTTVRRSRRIILFPVPFPGHLNPMLQLANVLYSKGFSITILHTRFNAPKTSNYPHFTFVFVLDNDPQDEWLRNLPTHGPGVGARIPVINKHGAEKLRKTLEELLASSESDEEISCLITDALWYFAQPVADSLGLRRLVLQTGSLFNFHCLVSLPQFEQQGYLDPSDQTNLDEPAEGFPLLKVKDIKRAYSHWQESKPILEKMVKQTKESSGVIWNSFKELEESELETVWREIPAPSFLIPLPKHYRASSTSLLDTDRSVFDWLDQQPPESVLYVGFGSQSEVTPEEFLTIAEGLVRSGVPFLWLVRPGFVKGEAWIEVLPEGFLGPRGRIVKSAPQQEVLAHPAIGAFWTHGGWNGTMESVCEGVPMIFSDFGLDQPLNARYMSEKLKIGVYLDNGWDPEEIAAAIRLVMRDEAGAPIRQNARELKQKADRSIMKGGSSYESLESLVSYISSL

SEQ ID NO: 481 - protein, 457 aa, synthetic construct
MPNTNTTVRRRRRIILFPVPFPGHLNPMLQLANVLYSKGFSITILHTRFNKPKTSNYPHFTFVEVLDNDPQDEWLRNLPTHGPGVGARIPVINKHGAEKFKEILENLMASSESDEEISCLITDALWYFAEEVAKSLNLRRLVLQTGSLFNFHCLVSLPQFEKLGYLDPSNQKDLDKPAEGFPLLKVKDIKRAYSHWQESKPILEKMVKQTKESSGVIWNSFKELEESELETVWREIPAPSFLIPLPKHYRASSTSLLDTDRSVFDWLATQPPESVLYVGFGSQSEVTPEEFLTIARGLVLSGVPFLWLVRPGFVKGQAWIEVLPDGFLGPQGRIVKSAPQQEVLAHPAIGAFWTHGGWNGTMESVCEGVPMIFSDFGLDQPLNARYMSEKLKIGVYLDEGWDPERIAAAIRLVMRDEAGKPIRENAKVLKQKADRSIMKGGSSYESLESLVSYISSL

SEQ ID NO: 482 - protein, 457 aa, synthetic construct
MPNTNTTVRRRLRIILFPVPFPGHLNPMLQLANVLYSKGFSITILHTRFNAPKTSNYPHFTFVFVLDNDPQDEWLRNLPTHGPGVGARIPVINKHGAEPLKKVLEELIAASESDERIACLITDALWYFAQEVADELNLPRLVLQTGSLFNFHCLVSLPKFEKLGYLNPSDNTNLDEPAEGFPLLKVKDIKRAYSHWQESKPILEKMVKATKASSGVIWNSFKELEESELETVWREIPAPSFLIPLPKHYRASSTSLLDTDRSVFEWLDQQPPRSVLYVGFGSQSEVTPEEALKIAEGLVRSGIPFLWLVRPGFVKGQAWIEVLPEGFLGERGRIVKSAPQQEVLAHPAIGAFWTHGGWNGTMESVCEGVPMIFSDFGLDQPLNATYMSRKLKIGVYLDNGFNPEEIANAIRLVMRDEKGKPIRENARKLKEKADRSIMKGGSSYESLESLVSYISSL

SEQ ID NO: 483 - protein, 457 aa, synthetic construct
MPNTNTTVRRRRRIILFPVPFPGHLNPMLQLANVLYSKGFSITILHTRFNAPKTSNYPHFTFVFVLDNDPQDEWLRNLPTHGPGVGARIPVINKHGAERFRKILEELMAASESDEEISCLITDALWYFAEEVAKSLNLPRLVLQTGSLFNFHCLVSLPKFEELGYLDPSNQTRLDEPAEGFPELKVKDIKRAYSHWQESKPILEKMVKATKESSGVIWNSFKELEESELETVWREIPAPSFLIPLPKHYRASSTSLLDTDRSVFDWLAQQPPRSVLYVGFGSQSEVTPEEALTIAEGLVRSGIPFLWLVRPGFVKGQAWIEVLPDGFLGPAGRIVKSAPQQEVLAHPAIGAFWTHGGWNGTMESVCEGVPMIFSDFGLDQPLNARYMSEVLKIGVYLDEGFDPERIAAAIRLVMRDPEGEPIRENARKLKEKADRSIMKGGSSYESLESLVSYISSL

SEQ ID NO: 484 - protein, 457 aa, synthetic construct
MPNTNTTVRRRRTIILFPVPFPGHLNPMLQLANVLYSKGFKIIILHTKFNAPKTSNYPHFTFVEVLDNDPQDEWLRNLPTHGPGVGARIPVINKHGAEEFRKILEELIASSESDEEISCLITDALWYFTQDVADSLNLRRLVLQTGSLFNFHCLVSLPKFEELGLLDPSDTTGLEEPVEGFPLLKRKDIKRAYSHWQESKPILEKMVKQTKASSGVIWNSFKELEESELETVWREIPAPSFLIPLPKHYRASSTSLLDTDRSVFDWLDQQPPESVLYVGFGSQSEVTPEEFLTIMEGLVKSGIPFLWLVRPGFVKGKAWIEVIPEGFGGEKGRIVKSAPQQEVLAHPAIGAFWTHGGWNGTMESVCEGVPMIFSDFGLDQPLNARYMSEKLKIGVYLDNGWNPEEIAAAIRYVMRDAAGEPIRKNAKELKQKADRSIMKGGSSYESLESLVSYISSL

SEQ ID NO: 485 - protein, 457 aa, synthetic construct
MPNTNTTVRRKRRIILFPVPFPGHLNPMLQLANVLYSKGFSITILHTNFNKPKTSNYPHFTFIDVLDNDPQDEWLRNLPTHGPGVGARIPVINKHGAEQFRKTLEELIASSESDETISCLITDALWYFAQDVADSLNLRRLVLQTGSLFNFHCLVSLPLFEKLGYLDPSDTSNLEEPVEGFPYLKVKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVTPEEFLTIAEGLVKSGIPFLWLVRPGFVKGRAWIEVLPEGFLGEKGRIVKSAPQQEVLAHPAIGAFWTHGGWNGTMESVCEGVPMIFSDFGLDQPLNARYMSDVLKIGVYLDDGWDPDAIAAAIRYVMRDAAGEPIRENARVLKQKADRSIMKGGSSYESLESLVSYISSL

SEQ ID NO: 486 - protein, 457 aa, synthetic construct
MPNTNTTVRRTRRIILFPVPFPGHLNPMLQLANVLYSKGFSITILHTRFNRPKTSNYPHFTFVEVLDEDPQDEWLRNLPTHGPGVGARIPVINKHGAERFRAVLEELIAASESDEEVACLITDALWYFAQDVADSLNLPRLVLQTGSLFNFHCLVSLPHFEELGYLDPDNTTNLDEPAVGFPLLKVKDIKRAYSHWQESKPILEKMVKATKASSGVIWNSFKELEESELETVWREIPAPSFLIPLPKHYRASSTSLLDTDRSVFDWLATQPPRSVLYVGFGSQSEVTPEEFLNIVRGLVKSGIPFLWLVRPGFVKGYAWVEPIPEGFLGEKGRIVKSAPQQEVLAHPAIGAFWTHGGWNGTMESVCEGVPMIFSDFGLDQPLNARYMSEKLKVGVYLDNGFDPDAIAAAIRYVMVDPEGAPIRENALKLKEKADRSIMKGGSSYESLESLVSYISSL

SEQ ID NO: 487 - protein, 457 aa, synthetic construct
MPNTNTTVRRRRRIVLFPVPFPGHLNPMLQLANVLYSKGFSITILHTKFNKPKTSNYPHFTFVEVLDNDPQDEWLRNLPTHGPGVGARIPVINKHGAEKLKETLEKLIEASESDEEISCLITDALWYFAQDVADSLNLRRLVLQTGSLFNFHCLVSLPEFEKLGYLDPNDTSGLDEPVPGFPYLRRKDIKRAYSHWQESKPILEKMVKQTKLSSGVIWNSFKELEESELETVWREIPAPSFLIPLPKHYRASSTSLLDTDRSVFDWLDKQPPESVLYVGFGSQSEVTEEEFLKIVEGLVKSGIPFLWLVRPGFVKGYDWIEPIPEGFLGEKGRIVKSAPQQEVLAHPAIGAFWTHGGWNGTMESVCEGVPMIFSDFGLDQPLNARYMSDVLKVGVYLDNGWDPDEIAAAIRYVMRDPAGAPIRENAKVLKQKADRSIMKGGSSYESLESLVSYISSL

SEQ ID NO: 488 - protein, 457 aa, synthetic construct
MPNTNTTVRRSRRIILFPVPFPGHLNPMLQLANVLYSKGFSITIFHTRFNAPKTSNYPHFTFVFILDNDPQDEWLRNLPTHGPGVGARIPVINKHGAERFKKELEKLIEASESDERVACLITDALWYFALDVAKSLNLPRLVLQTGSLFNFHCLVSLPQFEKLGYLDPSNTTNLDEPVEGFPLLKVKDIKRAYSHWQESKPILEKMVKATKESSGVIWNSFKELEESELETVWREIPAPSFLIPLPKHYRASSTSLLDTDRSVFAWLDTQPPRSVLYVGFGSQSEVTPEEFLNIVRGLVKSGIPFLWLVRPGFVKGYEWIEPIPEGFLGEKGRIVKSAPQQEVLAHPAIGAFWTHGGWNGTMESVCEGVPMIFSDFGLDQPLNARYMSEKLHVGVYLDEGFDPDRIAAAIRLVMRDPEGEPIRRNALELKEKADRSIMKGGSSYESLESLVSYISSL

SEQ ID NO: 489 - protein, 457 aa, synthetic construct
MPNTNTTVRRTRRIILFPVPFPGHLNPMLQLANVLYSKGFSITILHTKFNAPKTSNYPHFTFVFVLDNDPQDEWLRNLPTHGPGVGARIPVINKHGAEELKKTLEKIIEASESDEEVSCLITDALWYFAQDVAKSLNLRRLVLQTGSLFNFHCLVSLPKFEELGLLDPENTTNLDEPAPGFPWLKNKDIKRAYSHWQESKPILEKMVKQTKESSGVIWNSFKELEESELETVWREIPAPSFLIPLPKHYRASSTSLLDTDRSVFAWLDTQPPRSVLYVGFGSQSEVTPEEFLAIVEGLVKSGVPFLWLVRPGFVKGYEWIEPIPEGFLGEKGRIVKSAPQQEVLAHPAIGAFWTHGGWNGTMESVCEGVPMIFSDFGLDQPLNARYMSEVLKVGVYLDRGWNPDEIAAAIRLVMRDPAGEPIRQNAQELKQKADRSIMKGGSSYESLESLVSYISSL

SEQ ID NO: 490 - protein, 457 aa, synthetic construct
MPNTNTTVRRRRTIILFPVPFPGHLNPMLQLANVLYSKGFSIIILHTNFNKPKTSNYPHFTFVEVLDNDPQDEWLRNLPTHGPGVGARIPVINKHGAEEFEKTLKKIIEASESDEEVSCLITDALWYFTQDVADKLNLRRLVLQTGSLFNFHCLVSLPKFEELGYLDPSNTENLDEPVEGFPILKRKDIKRAYSHWQESKPILEKMVKQTKASSGVIWNSFKELEESELETVWREIPAPSFLIPLPKHYRASSTSLLDTDRSVFAWLDTQPPESVLYVGFGSQSEVTEEEFLKIVEGLVKSGIPFLWLVRPGFVKGKDWIEPIPEGFLGEKGRIVKSAPQQEVLAHPAIGAFWTHGGWNGTMESVCEGVPMIFSDFGLDQPLNARYMSEVKKIGVYLDNGWNPDEIAAAIRYVMRDPAGAPIRENAKALKQKADRSIMKGGSSYESLESLVSYISSL

SEQ ID NO: 491 - protein, 457 aa, synthetic construct
MPNTNTTVRRRRRIILFPVPFPGHLNPMLQLANVLYSKGFSITILHTRFNAPKTSNYPHFTFVFVLDNDPQDEWLRNLPTHGPGVGARIPVINKHGAEKFKKVLEEIIEASESDEEVSCLITDALWYFAQDVADSLGLRRLVLQTGSLFNFHCLVSLPQFEKLGYLDPDDTTNLDEPAEGFPYLKVKDIKRAYSHWQESKPILEKMVKQTKESSGVIWNSFKELEESELETVWREIPAPSFLIPLPKHYRASSTSLLDTDRSVFAWLDRQPPESVLYVGFGSQSEVTPEEFLAIVDGLVKSGIPFLWLVRPGFVKGYDWIEPIPEGRLGERGRIVKSAPQQEVLAHPAIGAFWTHGGWNGTMESVCEGVPMIFSDFGLDQPLNARYMSEVLGVGVYLDNGWDPDEIAAAIRLVMRDPEGAPIRENAKALKQKADRSIMKGGSSYESLESLVSYISSL

SEQ ID NO: 492 - protein, 457 aa, synthetic construct
MPNTNTTVRRSRRIILFPVPFPGHLNPMLQLANVLYSKGFSITILHTRFNKPKTSNYPHFTFVEVLDNDPQDEWLRNLPTHGPGVGARIPVINKHGAERLKEVLEKIIAASESDETVSCLITDALWYFAQDVAKSLNLRRLVLQTGSLFNFHCLVSLPKFEELGYLDPSNTTRLDEPVEGFPELKVKDIKRAYSHWQESKPILEKMVKQTKESSGVIWNSFKELEESELETVWREIPAPSFLIPLPKHYRASSTSLLDTDRSVFAWLDTQPPESVLYVGFGSQSEVTPEEFLNIVDGLVKSGIPFLWLVRPGFVKGEEWIEVIPEGKLGEKGRIVKSAPQQEVLAHPAIGAFWTHGGWNGTMESVCEGVPMIFSDFGLDQPLNARYMSEVLKVGVYLDNGWNPDEIAKAIRYVMRDPEGKPIRENAKVLKQKADRSIMKGGSSYESLESLVSYISSL

SEQ ID NO: 493 - protein, 457 aa, synthetic construct
MPNTNTTVRRTRRIILFPVPFPGHLNPMLQLANVLYSKGFSITILHTRFNKPKTSNYPHFTFVEVLDEDPQDEWLRNLPTHGPGVGARIPVINKHGAEKLKKTLEQLIAASESDETVSCLITDALWYFAEDVAKSLNLRRLVLQTGSLFNFHCLVSLPQFEKLGYLDPSNTTNLDQPAEGFPLLKVKDIKRAYSHWQESKPILEKMVKQTKESSGVIWNSFKELEESELETVWREIPAPSFLIPLPKHYRASSTSLLDTDRSVFAWLDTQPPRSVLYVGFGSQSEVTPKEFLEIVRGLVKSGVPFLWLVRPGFVKGYDWIEPIPEGLLGEKGRIVKSAPQQEVLAHPAIGAFWTHGGWNGTMESVCEGVPMIFSDFGLDQPLNARYMSEKLKVGVYLDEGWDPDRIAAAIRLVMEDPEGEPIRENAKVLKQKADRSIMKGGSSYESLESLVSYISSL

SEQ ID NO: 494 - protein, 457 aa, synthetic construct
MPNTNTTVRRRLRIILFPVPFPGHLNPMLQLANVLYSKGFKITIFHTRFNAPKTSNYPHFTFVEVLDNDPQDEWLRNLPTHGPGVGARIPVINKHGAERFKAELEKLIAASESDEKVACLITDALWYFTQDVADSLNLPRLVLQTGSLFNFHCLVSLPKFEELGLLDPSDTTNLDEPVPGFPILKRKDIKRAYSHWQESKPILEKMVKATKASSGVIWNSFKELEESELETVWREIPAPSFLIPLPKHYRASSTSLLDTDRSVFAWLDTQPPESVLYVGFGSQSEFTVEEFLAIVEGLVKSGIPFLWLVRPGFVKGKDWVEPIPEGFLGEKGRIVKSAPQQEVLAHPAIGAFWTHGGWNGTMESVCEGVPMIFSDFGLDQPLNARYMSRVKKIGVYLDNGFNPDEIAEAIRYVMRDPEGEPIRENAKELKEKADRSIMKGGSSYESLESLVSYISSL

SEQ ID NO: 495 - protein, 457 aa, synthetic construct
MPNTNTTVRRRLTIILFPVPFPGHLNPMLQLANVLYSKGFKIIILHTKFNAPKTSNYPHFTFVEILDNDPQDEWLRNLPTHGPGVGARIPVINKHGAEEFKKTLRKIIEASESDERVACLITDALWYFTQDVAKSLNLPRLVLQTGSLFNFHCLVSLPKFEELGLLDPENTENLDEPVPGFPLLKRKDIKRAYSHWQESKPILEKMVKETKASSGVIWNSFKELEESELETVWREIPAPSFLIPLPKHYRASSTSLLDTDRSVFAWLDTQPPESVLYVGFGSQSEFTPEEFLKIVEGLVKSGIPTLWLVRPGFVKGYDWIEPIPEGFLGEKGRIVKSAPQQEVLAHPAIGAFWTHGGWNGTMESVCEGVPMIFSDFGLDQPLNARYMSEKLKVGVYLDNGFDPDEIAAAIRYVMRDPAGAPIRENAKKLKEKADRSIMKGGSSYESLESLVSYISSL

SEQ ID NO: 496 - protein, 457 aa, synthetic construct
MPNTNTTVRRDRRIILFPVPFPGHLNPMLQLANVLYSKGFSITILHTRFNRPKTSNYPHFTFVEILDNDPQDEWLRNLPTHGPGVGARIPVINKHGAEPLKKTLEQIIAASESDERVSCLITDALWYFAQDVAKSLNLRRLVLQTGSLFNFHCLVSLPHFEQLGYLDPSNTTNLDQPAVGFPMLKVKDIKRAYSHWQESKPILEKMVKQTKESSGVIWNSFKELEESELETVWREIPAPSFLIPLPKHYRASSTSLLDTDRSVFAWLDTQPPESVLYVGFGSQSEVTVEEFLRIVEGLVKSGIPFLWLVRPGFVKGYEWIEPIPEGRLGERGRIVKSAPQQEVLAHPAIGAFWTHGGWNGTMESVCEGVPMIFSDFGLDQPLNARYMSEKLKVGVYLDNGWDPEEIAAAIRLVMRDPAGEPIRRNAKELKQKADRSIMKGGSSYESLESLVSYISSL

SEQ ID NO: 497 - protein, 457 aa, synthetic construct
MPNTNTTVRRDRRIILFPVPFPGHLNPMLQLANVLYSKGFSITIFHTNFNKPKTSNYPHFTFVNILDNDPQDEWLRNLPTHGPGVGARIPVINKHGAEKFKKALEELIEASESDERVSCLITDALWYFAEDVAKSLNLRRLVLQTGSLFNFHCLVSLPLFEKLGYLDPSNTTNLDEPAVGFPLLKVKDIKRAYSHWQESKPILEKMVKQTKASSGVIWNSFKELEESELETVWREIPAPSFLIPLPKHYRASSTSLLDTDRSVFDWLATQPPESVLYVGFGSQSEVTPEEFLAIVEGLVKSGVPFLWLVRPGFVKGYEWIEPIPEGKLGERGRIVKSAPQQEVLAHPAIGAFWTHGGWNGTMESVCEGVPMIFSDFGLDQPLNARYMSEKLHIGVYLDNGWDPEAIAAAINLVMRDPAGAPIRENAKVLKQKADRSIMKGGSSYESLESLVSYISSL

SEQ ID NO: 498 - protein, 457 aa, synthetic construct
MPNTNTTVRRERRIILFPVPFPGHLNPMLQLANVLYSKGFSITIFHTNFNKPKTSNYPHFTFVEVLDNDPQDEWLRNLPTHGPGVGARIPVINKHGAEKLKAALEEEIAASESDERVSCLITDALWYFAQDVADSLNLRRLVLQTGSLFNFHCLVSLPKFEELGYLDPDNTTNLDEPAEGFPMLKVKDIKRAYSHWQESKPILEKMVKQTKASSGVIWNSFKELEESELETVWREIPAPSFLIPLPKHYRASSTSLLDTDRSVFAWLDTQPPRSVLYVGFGSQSEVTREEFLNIVRGLVASGVPFLWLVRPGFVKGEDWIEPIPEGFLGERGRIVKSAPQQEVLAHPAIGAFWTHGGWNGTMESVCEGVPMIFSDFGLDQPLNARYMSEKLKVGVYLDEGWDPERIAAAIRLVMRDPAGAPIRENALVLKQKADRSIMKGGSSYESLESLVSYISSL

SEQ ID NO: 499 - protein, 457 aa, synthetic construct
MPNTNPTTVRRRTVVMFPVPFPGHLNPMLQLANVLYRRGFKIVILYTNFNAPKTSLYPHFQFVQILDNDPQPEWLRNLPTTGPGVGARIPVINKHGAAPFKAALEACIAATPSDEEVACVITDALWYFAGPVTEELNLKRLVLQTGSLFNFHCLVCLPEFLELGLLDPNTKHADDAPVPGYPFLRVRDIRRAYSHIQESKPILMKMVEETKRSSGVIWNSAKELEESELETIQREIPAPSFLLPLPKHYRASSTSLLDTDPSTAAWLDTQPPRSVLYVGFGSQSSLEPEEFLALAEGLVRSGLPFLWLVRPGFVKGKEWIELLPDGFLGERGRIVKSAPQQEVLAHPAIGAFWTHGGWNGTMEAVCEGVPMIFSDFGLDQPLNARYMAEVKKIGVYLDEGFEVERVVEAIHRVMREPEGEVYRRNAQELKDKLDRSIAPGGSSYESLERLQSYISSL

SEQ ID NO: 500 - protein, 457 aa, synthetic construct
MPNTNTTVRRRRRIILFPVPFPGHLNPMLQLANVLYSKGFSITILHTNFNAPKTSNYPHFTFVFILDNDPQDEWLRNLPTHGPGVGARIPVINKHGAEEFRKVLEEIISASESDETVSCLITDALWYFAQDVAKSLNLRRLVLQTGSLFNFHCLVSLPHFEELGLLDPENQTDLDKPAPGFPELKVKDIKRAYSHWQESKPILEKMVKQTKESSGVIWNSFKELEESELETVWREIPAPSFLIPLPKHYRASSTSLLDTDRSVFQWLDQQPPRSVLYVGFGSQSEVEPKDFLAIAEGLVRSGLPFLWLVRPGFVKGHEWIEVLPPGFLGPRGRIVKSAPQQEVLAHGAIGAFWTHGGWNGTMESVCEGVPMIFSDFGLDQPLNARYMSEVLKVGVYLDEGWEPERIAAAARLVMVDEAGAYIRENARELKQKADRSIMKGGSSYESLESLVSYISSL

SEQ ID NO: 501 - protein, 457 aa, synthetic construct
MPNTNTTVRRRRRIILFPVPFPGHLNPMLQLANVLYSKGFSITILHTRFNAPKTSNYPHFTFVFILDNDPQDEWLRNLPTHGPGVGARIPVINKHGAEELRKTLEELISASESDERVACLITDALWYFAQEVADSLNLRRLVLQTGSLFNFHCLVSLPKFEELGYLDPSDQRDLDKPAPGFPELKVKDIKRAYSHWQESKPILEKMVKATKESSGVIWNSFKELEESELETVWREIPAPSFLIPLPKHYRASSTSLLDTDRSVFAWLDQQPPRSVLYVGFGSQSEVEPEDALKIAEGLVKSGLPFLWLVRPGFVKGSTWIEVLPPGFLGERGRIVKSAPQQEVLAHPAIGAFWTHGGWNGTMESVCEGVPMIFSDFGLDQPLNARYMSEVLHVGVYLDNGFDPDEIARAARLVMVDPEGAYIRENARELKEKADRSIMKGGSSYESLESLVSYISSL

SEQ ID NO: 502 - protein, 457 aa, synthetic construct
MPNTNTTVRRRRRIILFPVPFPGHLNPMLQLANVLYSKGFSITILHTNFNKPKTSNYPHFTFREILDNDPQDEWLRNLPTHGPGVGARIPVINKHGAEEFRKTLEELISASESDEEVSCLITDALWYFTQDVADSLNLRRLVLQTGSLFNFHCLVSLPEFDKLGLLDPSDKTDLDKPAPGFPMLKRKDIKRAYSHWQESKPILEKMVKQTKASSGVIWNSFKELEESELETVWREIPAPSFLIPLPKHYRASSTSLLDTDRSVFEWLDTQPPSSVLYVGFGSQSEVSPEDFLAIAEGLVKSGQSFLWLVRPGFVKGSAWIEPLPEGFLGEKGRIVKSAPQQEVLAHPAIGAFWTHGGWNGTMESVCEGVPMIFSDFGLDQPLNARYMSDVKKIGVYLDDGWDPDAIAAAIRYVMVDPEGQAIRDNAKELKQKADRSIMKGGSSYESLESLVSYISSL

SEQ ID NO: 503 - protein, 457 aa, synthetic construct
MPNTNTTVRRRRRIVLFPVPFPGHLNPMLQLANVLYSKGFSITIIHTQFNAPKTSNYPHFTFRTILDNDPQDEWLRNLPTHGPGVGARIPVINKHGAEELRKMLEELISASESDEEVSCLITDALWYFAQDVAKSLGLRRLVLQTGSLFNFHCLVSLPKFDELGYLDPDDKTDLDKPVEGFPWLKVKDIKRAYSHWQESKPILEKMVKQTKESSGVIWNSFKELEESELETVWREIPAPSFLIPLPKHYRASSTSLLDTDRSVFAWLDRQPPRSVLYVGFGSQSEVDPATFLAIAEGLVRSGLSFLWLVRPGFVKGERWIEVLPDGFLGERGRIVKSAPQQEVLAHPAIGAFWTHGGWNGTMESVCEGVPMIFSDFGLDQPLNARYMSDVLHVGVYLDNGWEPEEIARAIRLVMVDPEGAHIRQNAAELKQKADRSIMKGGSSYESLESLVSYISSL

SEQ ID NO: 504 - protein, 457 aa, synthetic construct
MPNTNTTVRRTRRIILFPVPFPGHLNPMLQLANVLYSKGFSITIFHTEFNRPKTSNYPHFTFRTILDNDPQDEWLRNLPTHGPGVGARIPVINKHGAEQLRQELEELISASESDEEVSCLITDALWYFAQDVADSLNLRRLVLQTGSLFNFHCLVSLPQFEQQGWLDPENQTDLDQPAPGFPELRVRDIKRAYSHWQESKPILEKMVKQTKESSGVIWNSFKELEESELETVWREIPAPSFLIPLPKHYRASSTSLLDTDRSVFDWLDRQPPSSVLYVGFGSQSEVEPEEFLKIAEGLVKSGLSFLWLVRPGFVKGEEWIEVLPDGFLGEKGRIVKSAPQQEVLAHPAIGAFWTHGGWNGTMESVCEGVPMIFSDFGLDQPLNARYMSDVLHVGVYLRDGWEPEEIARAIRLVMVDEEGAYIRENARVLKQKADRSIMKGGSSYESLESLVSYISSL

SEQ ID NO: 505 - protein, 457 aa, synthetic construct
MPNTNTTVRRRRRIILFPVPFPGHLNPMLQLANVLYSKGFSITILHTNFNKPKTSNYPHFTFRFILDNDPQDEWLRNLPTHGPGVGARIPVINKHGAEELRKTLEELMSASESDEEVSCLITDALWYFAQDVAKSLNLRRLVLQTGSLFNFHCLVSLPQFDALGYLDPSNTTDLDKPVEGFPELKVKDIKRAYSHWQESKPILEKMVKQTKESSGVIWNSFKELEESELETVWREIPAPSFLIPLPKHYRASSTSLLDTDRSVFDWLDQQPPRSVLYVGFGSQSEVEPEDFLRIAEGLVRSGQPFLWLVRPGFVKGHTWIEVLPPGFLGERGRIVKSAPQQEVLAHPAIGAFWTHGGWNGTMESVCEGVPMIFSDFGLDQPLNARYMSEVLKVGVYLDRGWDPEEIARAIRLVMVDPEGAHIRENAEVLKQKADRSIMKGGSSYESLESLVSYISSL

SEQ ID NO: 506 - protein, 457 aa, synthetic construct
MPNTNTTVRRSRRIILFPVPFPGHLNPMLQLANVLYSKGFSITIIHTEFNRPKTSNYPHFTFVTILDNDPQDEWLRNLPTHGPGVGARIPVINKHGAEELRKTLEEIISASESDEEVSCLITDALWYFAQDVADSLNLRRLVLQTGSLFNFHCLVSLPKFEELGLLDPDDQSDLDKPVPGFPELLAKDIKRAYSHWQESKPILEKMVKQTKASSGVIWNSFKELEESELETVWREIPAPSFLIPLPKHYRASSTSLLDTDRSVFAWLDQQPPESVLYVGFGSQSEVEPEDFLRIAEGLVKSGLSFLWLVRPGFVKGETWIEVLPDGFLGERGRIVKSAPQQEVLAHPAIGAFWTHGGWNGTMESVCEGVPMIFSDFGLDQPLNARYMSEVLKVGVYLRNGWEPEEIARAIRLVMVDEEGAHIRENARELKQKADRSIMKGGSSYESLESLVSYISSL

SEQ ID NO: 507 - protein, 457 aa, synthetic construct
MPNTNTTVRRRRRIILFPVPFPGHLNPMLQLANVLYSKGFSITILHTNFNKPKTSNYPHFTFREILDNDPQDEWLRNLPTHGPGVGARIPVINKHGAEEFRKELEELMAASESDEEVSCLITDALWYFAQDVADSLNLRRLVLQTGSLFNFHCLVSLPKFDELGYLDPSDTTNLDEPVEGFPWLKRKDIKRAYSHWQESKPILEKMVKQTKLSSGVIWNSFKELEESELETVWREIPAPSFLIPLPKHYRASSTSLLDTDRSVFAWLDTQPPGSVLYVGFGSQSEVTPEDFLKIAEGLVKSGQPFLWLVRPGFVKGSAWIEPLPEGFLGEKGRIVKSAPQQEVLAHPAIGAFWTHGGWNGTMESVCEGVPMIFSDFGLDQPLNARYMSEVLKIGVYLDDGWEPEKIAAAIRLVMTDEAGAPMRENAAVLKQKADRSIMKGGSSYESLESLVSYISSL

SEQ ID NO: 508 - protein, 457 aa, synthetic construct
MPNTNTTVRRRRRIILFPVPFPGHLNPMLQLANVLYSKGFSITIFHTKFNAPKTSNYPHFTFREILDNDPQDEWLRNLPTHGPGVGARIPVINKHGADEFRKALEELMSASESDEEVSCLITDALWYFTQDVADSLNLRRLVLQTGSLFNFHCLVSLPKFEELGLLDPDDKTDLDKPVPGFPELKRKDIKRAYSHWQESKPILEKMVKQTKASSGVIWNSFKELEESELETVWREIPAPSFLIPLPKHYRASSTSLLDTDRSVFAWLDQQPPRSVLYVGFGSQSEVSPEDFLKIVEGLVLSGQPFLWLVRPGFVKGKEWIEPLPEGFLGEKGRIVKSAPQQEVLAHPAIGAFWTHGGWNGTMESVCEGVPMIFSDFGLDQPLNARYMSDVLKVGVYLDNGWDPEEIARAIRLVMVDEAGAPIRENAEALKQKADRSIMKGGSSYESLESLVSYISSL

SEQ ID NO: 509 - protein, 457 aa, synthetic construct
MPNTNTTVRRRRRIILFPVPFPGHLNPMLQLANVLYSKGFSITIIHTNFNKPKTSNYPHFTFRTILDNDPQDEWLRNLPTHGPGVGARIPVINKHGADQFRKILEELMSASESDEKVSCLITDALWYFALDVAKSLNLRRLVLQTGSLFNFHCLVSLPKFEELGLLDPDDKTGLDEPVPGFPWLKRKDIKRAYSHWQESKPILEKMVKQTKASSGVIWNSFKELEESELETVWREIPAPSFLIPLPKHYRASSTSLLDTDRSVFAWLDQQPPGSVLYVGFGSQSEVDPKTFLKIAEGLVKSGQKFLWLVRPGFVKGSDWIEPLPDGFLGEKGRIVKSAPQQEVLAHPAIGAFWTHGGWNGTMESVCEGVPMIFSDFGLDQPLNARYMSDVLKVGVYLDDGWEPEKIAAAIRKVMVDPEGAAIRENAAELKQKADRSIMKGGSSYESLESLVSYISSL

SEQ ID NO: 510 - protein, 457 aa, synthetic construct
MPNTNTTVRRTRRIVLFPVPFPGHLNPMLQLANVLYSKGFSITILHTRFNAPKTSNYPHFTFVFILDNDPQDEWLRNLPTHGPGVGARIPVINKHGAEELRKALEEIISASESDEEVSCLITDALWYFAQDVADSLGLKRLVLQTGSLFNFHCLVSLPRFEELGYLDPENQTDLDKPVEGFPELKVKDIKSAYSHWQESKPILEKMVKQTRESSGVIWNSFKELEESELETVQREIPAPSFLIPLPKHYRASSTSLLDTDRSVFAWLDRQPPGSVLYVGFGSQSEVDRETFLAIAEGLRRSGLPFLWLVRPGFVKGEEWIESLPPGFLGPRGRVVKSAPQQEVLAHPAIGAFWTHGGWNGTMEAVCEGVPMIFSDFGLDQPLNARYMSDVLGVGVYLDRGWDPEEIARAIRLVMVDPEGAPIRENARALKQKADRSIMKGGSSYESLESLVSYISSL

SEQ ID NO: 511 - protein, 457 aa, synthetic construct
MPNTNTTVRRTRRIILFPVPFPGHLNPMLQLANVLYSKGFSITILHTNFNKPKTSNYPHFTFRFILDNDPQDEWLRNLPTHGPGVGARIPVINKHGAERLKQILQQEIQASESDVQVSCLITDALWYFAQDVADSLNLKRLVLQTGSLFNFHCLVSLPEFEKLGYLDPDNKTDLDKPAKGFPELLVKDIKSAYSHWQESKPILEKMVKQTRASSGVIWNSFKELEESELETVQREIPAPSFLIPLPKHYRASSTSLLDTDRSVFQWLDTQPPGSVLYVGFGSQSEVDRATFLAIAEGLVRSGLSFLWLVRPGFVKGERWIEVLPPGFLGPRGRVVKSAPQQEVLAHPAIGAFWTHGGWNGTMEAVCEGVPMIFSDFGLDQPLNARYMSDVLKVGVYLRDGWDPERIAAAIRLVMVDPEGAPIRENARVLKQKADRSIMKGGSSYESLESLVSYISSL

SEQ ID NO: 512 - protein, 457 aa, synthetic construct
MPNTNTTVRRTRRIILFPVPFPGHLNPMLQLANVLYSKGFSITILHTKFNAPKTSNYPHFTFVYTLDNDPQDEWLRNLPTHGPGVGARIPVINKHGAEEFKKTLEEIISASESDEEVSCLITDALWYFALDVAKSLNLKRLVLQTGSLFNFHCLVSLPKFEELGLLDPENKENLDEPVPGFPELKAKDIKSAYSHWQESKPILEKMVKQTRASSGVIWNSFKELEESELETVQREIPAPSFLIPLPKHYRASSTSLLDTDRSVFDWLDTQPPGSVLYVGFGSQSEVDRETFLAIARGLVASGLPFLWLVRPGFVKGETWIEPLPPGFLGPRGRVVKSAPQQEVLAHPAIGAFWTHGGWNGTMEAVCEGVPMIFSDFGLDQPLNARYMSDVKKVGVYLENGWDPEAIARAIRLVMVDPEGAPIRENAKELKQKADRSIMKGGSSYESLESLVSYISSL

SEQ ID NO: 513 - protein, 457 aa, synthetic construct
MPNTNTTVRRTRRIILFPVPFPGHLNPMLQLANVLYSKGFSITIFHTNFNKPKTSNYPHFTFKFILDNDPQDEWLRNLPTHGPGVGARIPVINKHGAERFKKLLEEVISASESDVTVSCLITDALWYFAQDVADSLNLKRLVLQTGSLFNFHCLVSLPKFDELGYLDPDDKTRLDEPAEGFPELKVKDIKSAYSHWQESKPILEKMVKQTRASSGVIWNSFKELEESELETVQREIPAPSFLIPLPKHYRASSTSLLDTDRSVFQWLDTQPPGSVLYVGFGSQSEVDEATFLAIVDGLVASGLPFLWLVRPGFVKGKTWIEAIPPGYLGPRGRVVKSAPQQEVLAHPAIGAFWTHGGWNGTMEAVCEGVPMIFSDFGLDQPLNARYMSDVLKVGVYLDDGWDPAAIAAAIRLVMTDPAGAPIRENAKVLKQKADRSIMKGGSSYESLESLVSYISSL

SEQ ID NO: 514 - protein, 457 aa, synthetic construct
MPNTNTTVRRTRRIILFPVPFPGHLNPMLQLANVLYSKGFSITIIHTNFNAPKTSNYPHFTFRFILDNDPQDEWLRNLPTHGPGVGARIPVINKHGAERFRKILEEIISASESDEEVSCLITDALWYFAQEVADSLNLKRLVLQTGSLFNFHCLVSLPAFEALGLLDPADTTRLDEPVPGLPELKAKDIKSAYSHWQESKPILEKMVKQTRASSGVIWNSFKELEESELETVQREIPAPSFLIPLPKHYRASSTSLLDTDRSVFDWLDQQPPGSVLYVGFGSQSEVTREQFLAIARGLVASGLPFLWLVRPGFVKGEDWIEDLPPGFLGPRGRVVKSAPQQEVLAHGAIGAFWTHGGWNGTMEAVCEGVPMIFSDFGLDQPLNARYMSDVKKIGVYLDNGWDPEEIAAAIRYVMTDPAGAPIRENAKELKQKADRSIMKGGSSYESLESLVSYISSL

SEQ ID NO: 515 - protein, 457 aa, synthetic construct
MPNTNTTVRRTRRIILFPVPFPGHLNPMLQLANVLYSKGFSITILHTNFNAPKTSNYPHFTFRFILDNDPQDEWLRNLPTHGPGVGARIPVINKHGAERLRKELEEIISASESDETVSCLITDALWYFAQDVAKSLNLKRLVLQTGSLFNFHCLVSLPRFEELGYLDPSDNTRLDEPAEGLPELLVKDIKSAYSHWQESKPILEKMVKQTRESSGVIWNSFKELEESELETVQREIPAPSFLIPLPKHYRASSTSLLDTDRSVFDWLAQQPPRSVLYVGFGSQSEVDRETALAIARGLVASGLPFLWLVRPGFVKGERWIEVLPPGFLGPRGRVVKSAPQQEVLAHPAIGAFWTHGGWNGTMEAVCEGVPMIFSDFGLDQPLNARYMSDVLRVGVYLRNGWDPEAIAAAIRLVMVDPAGAPIRENARRLKQKADRSIMKGGSSYESLESLVSYISSL

SEQ ID NO: 516 - protein, 457 aa, synthetic construct
MPNTNTTVRRTRRIILFPVPFPGHLNPMLQLANVLYSKGFSITIIHTNFNKPKTSNYPHFTFRTILDNDPQDEWLRNLPTHGPGVGARIPVINKHGAERFRKLLEEIISASESDEEVACLITDALWYFTQSVADSLNLPRLVLQTGSLFNFHCLVSLPKFEELGYLDPNDKTRLDEPAEGFPWLKVKDIKSAYSHWQESKPILEKMVKATRASSGVIWNSFKELEESELETVQREIPAPSFLIPLPKHYRASSTSLLDTDRSVFDWLATQPPGSVLYVGFGSQSEFDEATFLAIAEGLVASGLPALWLVRPGFVKGKAWIESLPPGFLGPRGRVVKSAPQQEVLAHPAIGAFWTHGGWNGTMEAVCEGVPMIFSDFGLDQPLNARYMSDVLKVGVYLDNGFDPEAIAAAIRLVMTDPAGAPIRENARELKEKADRSIMKGGSSYESLESLVSYISSL

SEQ ID NO: 517 - protein, 457 aa, synthetic construct
MPNTNTTVRRTRRIILFPVPFPGHLNPMLQLANVLYSKGFSITIIHTNFNAPKTSNYPHFTFVTILDNDPQDEWLRNLPTHGPGVGARIPVINKHGAERLRKILEEIISASESDVEVSCLITDALWYFAQDVADSLNLKRLVLQTGSLFNFHCLVSLPKFEELGYLDPSDKTGLDEPAEGFPWLKVKDIKSAYSHWQESKPILEKMVKQTRASSGVIWNSFKELEESELETVQREIPAPSFLIPLPKHYRASSTSLLDTDRSVFAWLDTQPPGSVLYVGFGSQSEVTREEFLAIVDGLVASGLPFLWLVRPGFVKGEAWIEAIPPGYLGPRGRVVKSAPQQEVLAHPAIGAFWTHGGWNGTMEAVCEGVPMIFSDFGLDQPLNARYMSDVLKVGVYLDNGWDPEEIAAAIRRVMVDPEGAPIRENARELKQKADRSIMKGGSSYESLESLVSYISSL

(19) United States
(12) Patent Application Publication
(10) Pub. No.: US 2026/0365999 A1
(43) Pub. Date: Nov. 19, 2026

(54) COMPOSITIONS AND METHODS FOR PRODUCING REBAUDIOSIDE M

(71) Applicant: Arzeda Corp., Seattle, WA (US)

(72) Inventors: Kyle ROBERTS, San Marcos, CA (US); Alexandre ZANGHELLINI, Memphis, TN (US); Daniela GRABS, Seattle, WA (US); Niklas KRISTIANSEN, Sammamish, WA (US); James HAVRANEK, Seattle, WA (US)

(21) Appl. No.: 19/107,436

(22) PCT Filed: Sep. 1, 2023
(86) PCT No.: PCT/US2023/073344

§ 371 Date: Feb. 27, 2025

Related U.S. Application Data
(60) Provisional application No. 63/374,461, filed on Sep. 2, 2022.

(51) Int. Cl. C12N 9/10; C12N 11/00; C12N 15/70; C12N 15/81; C12P 19/18; C12P 19/56; C12R 1/19; C12R 1/84

(52) U.S. Cl. CPC C12N 9/1051; C12N 11/00; C12N 15/70; C12N 15/815; C12P 19/18; C12P 19/56; C12R 2001/19; C12R 2001/84; C12Y 204/01013

(57) ABSTRACT

The present disclosure provides enzymes and a method to use those enzymes to transfer a sugar moiety to a substrate steviol glycoside. The designed beta-1,3-glycosyltransferases can add beta-1,3 linked glucose monomers to steviol glycosides. Specifically, the designed beta-1,3-glycosyltransferases are used in a one-pot reaction with a beta-1,2-glycosyltransferase and sucrose synthase to convert stevioside and/or Reb A into Reb M.

### CROSS-REFERENCE TO RELATED APPLICATIONS

This application claims the benefit of U.S. Provisional Patent Application No. 63/374,461, filed on Sep. 2, 2022, the content of which is herein incorporated by reference in its entirety.

### REFERENCE TO AN ELECTRONIC SEQUENCE LISTING

The contents of the electronic sequence listing (ARZE_038_01WO_Se-qList_ST26.xml; Size: 681,823 bytes; and Date of Creation: Sep. 1, 2023) are herein incorporated by reference in its entirety.

### FIELD OF THE DISCLOSURE

The present disclosure relates to enzymes and biocatalytic processes for producing steviol glycosides. The present disclosure particularly relates to the use of glycosyltransferases that can transfer a glucose moiety from an ADP-glucose sugar donor to steviol glycosides.

### BACKGROUND

Excess sugar consumption has been linked to worldwide health epidemics including diabetes and heart disease. Healthcare systems incur exorbitant costs associated with treating these diseases. Replacing added sugar in food with a low calorie, high-intensity sweetener would have significant health and economic impact.

The species Stevia rebaudiana is commonly grown for its sweet leaves, which have traditionally been used as a sweetener. Stevia extract is 200-300 times sweeter than sugar and is used commercially as a high intensity sweetener. The main glycoside components of stevia leaf are steviosides and rebaudiosides. Over ten different steviol glycosides are present in appreciable quantities in the leaf. The principal sweetening compounds are stevioside and rebaudioside A. Rebaudioside A (Reb A) is considered higher value compared to stevioside, because of its increased sweetness and decreased bitterness.

Further, when compared to Reb A, the sweetness and bitterness profile of rebaudioside M (Reb M) is improved. Reb M, however, is present at very low quantities in the stevia leaf. Accordingly, improved methods for converting glucose sources into low-order steviol glycosides are required.

### SUMMARY OF THE INVENTION

The present disclosure provides enzymes, particularly non-natural enzymes, and methods to use those enzymes to transfer a sugar moiety to a substrate steviol glycoside. Specifically, beta-1,3-glycosyltransferases (also referred to herein as “B13GTs”) are disclosed that can glycosylate the C3′ of the 13-O-glucose and/or the 19-O-glucose of a Steviol glycoside substrate. The present disclosure provides methods to use the disclosed beta-1,3-glycosyltransferases in combination with a sucrose synthase (SuSy) and optionally a beta-1,2-glycosyltransferase (B12GT) to make Reb M.

In contrast to native glycosyltransferases, the disclosure provides glycosyltransferase polypeptides that can utilize adenosine diphosphate (ADP)-glucose as the sugar donor to glycosylate the C3′ of the 13-O-glucose and/or 19-O-glucose of a steviol glycoside substrate. The disclosure provides glycosyltransferase polypeptides that comprise an amino acid sequence that is at least 60%, at least 65%, at least 70%, at least 75%, at least 80%, at least 85%, at least 90%, at least 95%, at least 98%, at least 99%, or 100% identical to an amino acid sequence selected from the group consisting of SEQ ID NOs: 2-517. The glycosyltransferase polypeptide may comprise, or consist of, an amino acid sequence selected from the group consisting of SEQ ID NOs: 2-517. The polypeptides may comprise one or more peptide tags used for solubility, expression and/or purification; for example, a polyhistidine tag of between 4 and 10 histidine residues, preferably 6 histidine residues. Other suitable tags include, but are not limited to, glutathione S-transferase (GST), FLAG, maltose binding protein (MBP), calmodulin binding peptide (CBP), and Myc tag. Suitable linkers include, but are not limited to, polypeptides composed of glycine and serine, such as GSGS, polyglycine linkers, EAAAK repeats, and sequences containing cleavage sites for enzymes such as factor Xa, enterokinase, and thrombin.

The disclosure additionally provides a method to utilize the disclosed B13GTs to make steviol glycosides, specifically Reb M. Starting with a feed containing Reb A and/or stevioside, the enzymes B13GT, B12GT and SuSy can be combined with sucrose, ADP and the steviol glycoside feed to convert the Reb A and/or stevioside to Reb M. In some embodiments, the method comprises contacting a stevia leaf extract purified to contain greater than 50% Reb A (RA50), ADP, and sucrose with a beta-1,3-glycosyltransferase, beta-1,2 glycosyltransferase and sucrose synthase to make Reb M. In another embodiment, the method comprises contacting a stevia leaf extract purified to contain greater than 60% Reb A (RA60), ADP, and sucrose with a beta-1,3-glycosyltransferase, beta-1,2 glycosyltransferase, and sucrose synthase to make Reb M. In another embodiment, the method comprises contacting a stevia leaf extract purified to contain greater than 90% stevioside (RS90), ADP, and sucrose with a beta-1,3-glucosyltransferase, beta-1,2-glucosyltransferase, and sucrose synthase to make Reb M. In another embodiment, the method comprises contact a stevia leaf extract purified to contain greater than 99% stevioside (RS99), ADP, and sucrose with a beta-1,3-glucosyltransferase, beta-1,2-glucosyltransferase, and sucrose synthase to make Reb M.

### BRIEF DESCRIPTION OF THE DRAWINGS

The accompanying drawings are included to provide a further understanding of the disclosure. The drawings illustrate embodiments of the disclosure and together with the description, serve to explain the principles of the embodiments of the disclosure.

FIG. 1 Chemical structure of rebaudioside M.

FIG. 2 Partial steviol glycoside network showing conversion of stevioside and Reb A to Reb M through additions of glucose monomers catalyzed by a beta-1,2-glycosyltransferase (B12GT) and beta-1,3-glycosyltransferase (B13GT). For each molecule, the central hexagon represents the steviol glycoside core, circles represent beta-1,2-linked glucose monomers and squares represent beta-1,3-linked glucose monomers.

FIG. 3 SDS-PAGE gel of designed beta-1,3-glycosyltransferases expressed in Pichia pastoris and purified by immobilized metal affinity chromatography.

### DETAILED DESCRIPTION

The present disclosure provides enzymes and biocatalytic processes for preparing a composition comprising a target steviol glycoside by contacting a starting composition comprising a substrate steviol glycoside, sucrose, and nucleotide diphosphate (NDP) with one or more NDP-glycosyltransferase polypeptides and a sucrose synthase, thereby producing a composition comprising a target steviol glycoside comprising one or more additional glucose units than the substrate steviol glycoside.

### DEFINITIONS

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, ADP, GDP, UDP, CDP, and TDP are used to refer, respectively, to adenosine diphosphate, guanosine-5′-diphosphate, uridine diphosphate, cytidine diphosphate, and thymidine diphosphate.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by Clustal Omega® using default parameters.

As used herein, “biocatalysis” or “biocatalytic” refers to the use of natural catalysts, such as protein enzymes, to perform chemical transformations on organic compounds. Biocatalysis is alternatively known as biotransformation or biosynthesis. Both isolated and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, synthetic steviol glycosides, e.g. enzymatically glycosylated steviol glycosides and combinations thereof. For example, the term steviol glycosides includes the known steviol glycosides steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside E2, rebaudioside E3, rebaudioside H, rebaudioside L, rebaudioside K, rebaudioside J, rebaudioside M, rebaudioside D, rebaudioside N, rebaudioside O, rebaudioside Q, and rebaudioside AM.

As used herein, “starting composition” refers to any composition (generally an aqueous solution) containing one or more steviol glycosides, where the one or more steviol glycosides serve as the substrate for the biotransformation.

As used herein, the terms “polynucleotide” or “nucleic acid” are used interchangeably, unless indicated by context, and is used to refer to a polymeric form of nucleotides of any length, either ribonucleotides or deoxyribonucleotides, typically DNA.

As used herein, “expression” refers to either or both steps, depending on context, of the two-step process by which polynucleotides are transcribed into mRNA and the transcribed mRNA is subsequently translated into a polypeptides.

“Under transcriptional control” means that transcription of a polynucleotide, usually a DNA sequence, depends on its being operatively linked to an element that promotes transcription.

“Operatively linked” means that the polynucleotide elements are arranged in a manner that allows them to function in a cell; typically to produce polypeptides in the cell; for example the disclosure provides promoters operatively linked to the downstream sequences encoding polypeptides.

The term “encode” refers to the ability of a polynucleotide to produce an mRNA or a polypeptide if it can be transcribed to produce the mRNA and then translated to produce the polypeptide or a fragment thereof. In each case, the polynucleotide is referred to as encoding the mRNA and encoding the polypeptide. The antisense strand is the complement of such a nucleic acid, and the encoding sequence can be deduced therefrom. Similarly, a “coding sequence” refers to a region of a nucleic acid that encodes an mRNA or a polypeptide.

The term “promoter” as used herein refers to a control sequence that is a portion of a polynucleotide sequence that controls the initiation and rate of transcription of a coding sequence. An “enhancer” is a regulatory element that increases the expression of a target sequence. A “promoter/enhancer” is a polynucleotide with sequences that provide both promoter and enhancer functions.

The regulatory elements, e.g. enhancers and promoters, may be “homologous” or “heterologous.” A “homologous” regulatory element is one which is naturally linked with a given polynucleotide in the genome; for example, it may be the promoter found natively in the organism upstream of the encoded polypeptide. A “heterologous” regulatory element is one which is placed in juxtaposition to a polynucleotide by means of recombinant molecular biological techniques but is not a combination found in nature. Often, promoters, enhancers and other regulatory elements are heterologous so as to facilitate expression of a polypeptide in a host cell other than one in which a polypeptide naturally occurs. Thus, “heterologous expression”, as used herein, refers to producing an mRNA and/or a polypeptide in a host cell, such as a microorganism, where the polynucleotide is not found naturally or one or more regulatory elements are not naturally found operably linked to the polynucleotide in the host cell.

The term “polypeptide” is used here to refer to a molecule of two or more subunits of amino acids linked by peptide bonds. Typically, though not always, the polypeptides contain several hundred amino acids; for example, about 400 to about 900 amino acids.

A “plasmid” is a DNA molecule that is typically separate from and capable of replicating independently of the chromosomal DNA. In many cases, it is circular and double-stranded. It is known in the art that while plasmid vectors often exist as extrachromosomal circular DNA molecules, plasmid vectors may also be designed to be stably integrated into a host chromosome either randomly or in a targeted manner. Many plasmids are commercially available for varied uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Typically, the polypeptides disclosed herein are expressed from plasmids.

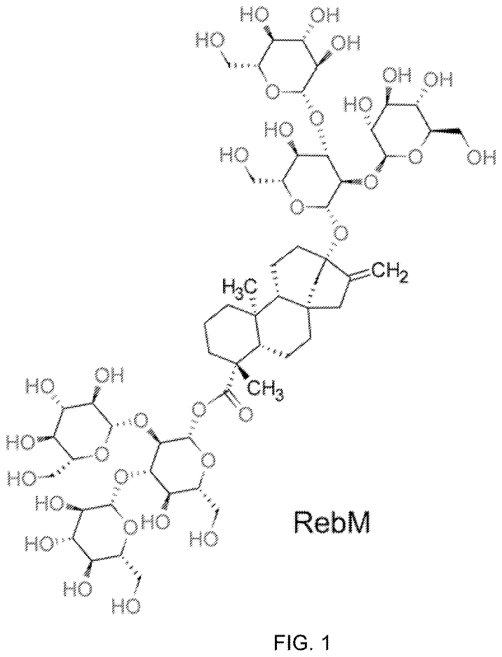

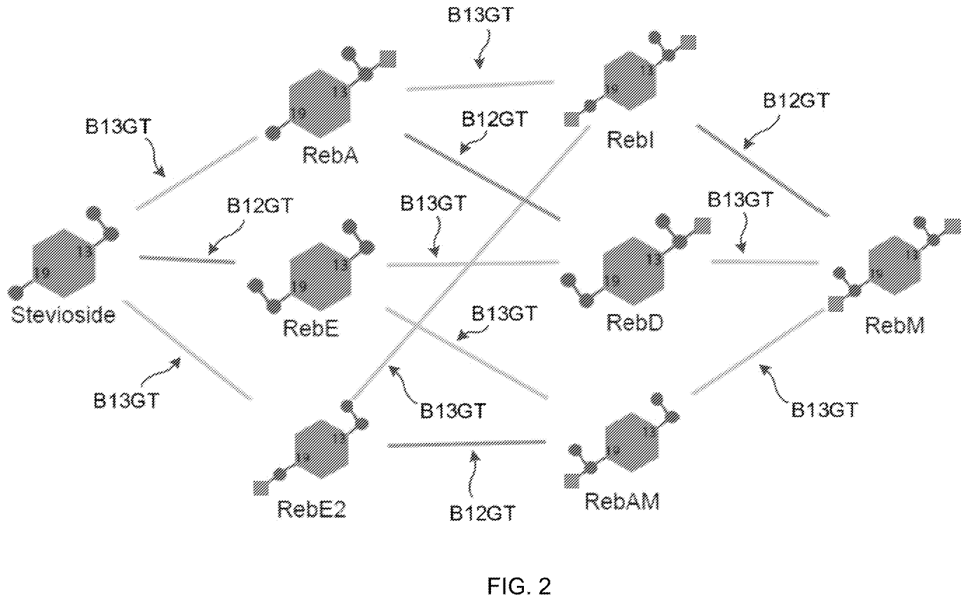

Reb M, the chemical structure of which is shown in FIG. 1, can be made by adding a beta-1,3-linked glucose monomer to the lower-order steviol glycosides Rebaudioside I (Reb I), or Reb AM or adding a beta-1,2-linked glucose monomer to Rebaudioside D (Reb D), as shown in FIG. 2. Native glycosyltransferases that perform these conversions use uridine diphosphate (UDP)-glucose as the glucose source for transferring to the lower-order steviol glycosides.

The present disclosure provides non-natural, engineered beta-1,3-adenosine diphosphate (ADP) glycosyltransferases (B13GTs) that can use an ADP-glucose sugar donor to add a beta-linked glucose monomer to the C3′ of the 13-O-glucose and/or 19-O-glucose of a steviol glycoside substrate. For example, the disclosed B13GTs can perform the following conversions: stevioside to Reb A, stevioside to Rebaudioside E2 (Reb E2), Reb A to Reb I, Rebaudioside E (Reb E) to Reb D, Reb E to Rebaudioside AM (Reb AM), Reb E2 to Reb I, Reb D to Reb M and Reb AM to Reb M. In a particular embodiment, the glycosyltransferase polypeptide is one of SEQ ID NOs: 2-517. In another embodiment, the glycosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or 100% identical to one of SEQ ID NOs: 2-517.

In bioinformatics, several methods have been developed to find and determine related polypeptide sequences. For example, percent sequence identity, position-specific scoring matrices (PSSMs), and hidden Markov models (HMMs) are all commonly employed to find sequences that are similar to a given query sequence. Percent sequence identity calculates the number of amino acids that are shared between two sequences. Percent sequence identity is calculated in the context of a given alignment between two sequences. Percentage identity may be calculated using the alignment program Clustal Omega (available at /www.ebi.ac.uk/Tools/msa/clustalo/) with default settings. The default transition matrix is Gonnet, gap opening penalty is 6 bits, and gap extension is 1 bit. Clustal Omega uses the HHalign algorithm and its default settings as its core alignment engine. The algorithm is described in Söding, J. (2005) ‘Protein homology detection by HMM-HMM comparison’. Bioinformatics 21, 951-960.

Position-specific scoring matrices (PSSMs) are a concise way to represent many related sequences. PSSMs are often generated using multiple sequence alignments. The sequence search tool PSI-BLAST generates PSSMs and uses them to search for related polypeptide sequences. A PSSM used to score polypeptide sequences is a matrix (i.e. table) composed of 21 columns by N rows, where N is the length of the related sequences. Each row corresponds to a position within the polypeptide sequence and each column represents a different amino acid (or gap) that the residue position can take on. Each entry in the PSSM represents a score for the specific amino acid at the specific position within the polypeptide sequence. A sequence can be scored with a PSSM by first aligning the sequence to a reference sequence, and then calculating the following sum:

$${S}_{PSSM} = {\sum }_{i = 1}^{N} PSSM ( i , {aa}_{i} ) ,$$

where i is the sequence position and aaᵢ is the amino acid at position i. Related polypeptide sequences will all have high PSSM scores, while unrelated sequences will yield low scores.

In some embodiments, the beta-1,3-glycosyltransferase polypeptide is prepared by expression in a host microorganism. Suitable host microorganisms include, but are not limited to, E. coli, Saccharomyces sp., Aspergillus sp., Pichia sp., Bacillus sp. In a particular embodiment, the glycosyltransferase is expressed in E. coli. In a particular embodiment, the glycosyltransferase is expressed in Pichia pastoris.

The B13GT polypeptide can be provided in any suitable form, including free, immobilized or as a whole cell system. The degree of purity of the glycosyltransferase polypeptide may vary, e.g., it may be provided as a crude, semi-purified or purified enzyme preparation(s). In one embodiment, the glycosyltransferase polypeptide is free. In another embodiment, the glycosyltransferase polypeptide is immobilized to a solid support, for example on an inorganic or organic support. In some embodiments, the solid support is derivatized cellulose, glass, ceramic, methacrylate, styrene, acrylic, a metal oxide, or a membrane. In some embodiments, the glycosyltransferase polypeptide is immobilized to the solid support by covalent attachment, adsorption, cross-linking, entrapment, or encapsulation.

In yet another embodiment, the B13GT polypeptide is provided in the form of a whole cell system, for example as a living fermentative microbial cell, or as dead and stabilized microbial cell, or in the form of a cell lysate.

The present disclosure provides a biocatalytic process for the preparation of a composition comprising one or more target steviol glycosides from a starting composition comprising one or more substrate steviol glycosides, wherein the target steviol glycoside comprises steviol glycosides with one or more additional glucose units than the substrate steviol glycoside. The biocatalytic process comprises contacting an engineered B13GT, a sucrose synthase (SuSy) (unless ADP-glucose is directly supplied), and optionally a B12GT with a starting composition containing one or more steviol glycosides, a non-UDP nucleotide diphosphate, and sucrose. In another embodiment, the biocatalytic process comprises contacting an engineered B13GT, a SuSy, and optionally a B12GT with a starting composition containing one or more steviol glycosides, a non-UDP nucleotide diphosphate, and sucrose. In some embodiments, the method comprises contacting RA50, ADP, and sucrose with an engineered B1,3 glycosyltransferase, a B12GT, and a SuSy to make Reb M. In another embodiment, the method comprises contacting RA60, ADP, and sucrose with an engineered B13GT, a B12GT, and a SuSy to make Reb M. In another embodiment, the method comprises contacting stevioside, ADP, and sucrose with an engineered B1,3 glycosyltransferase, a B12GT, and a SuSy to make Reb M. In another embodiment, the method comprises contacting Reb A, ADP, and sucrose with an engineered B13GT, a B12GT, and a SuSy to make Reb M. In another embodiment, the method comprises contacting RS90, ADP, and sucrose with an engineered B13GT, a B12GT, and a SuSy to make Reb M. In another embodiment, the method comprises contacting RS99, ADP, and sucrose with an engineered B13GT, B12GT, and a SuSy to make Reb M.

In one embodiment, the B13GT polypeptide is one of SEQ ID NOs: 2-517. In another embodiment, the glycosyltransferase polypeptide is a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or 100% identical to one of SEQ ID NOs: 2-517.

In one embodiment the sucrose synthase is any polypeptide with sucrose synthase activity. In another embodiment, the sucrose synthase is derived from an organism from the Bac-teria domain. In another embodiment, the sucrose synthase is derived from an organism from the Plantae kingdom. In another embodiment, the sucrose synthase is derived from an organism from the proteobacteria, deferribacteres, or cyanobacteria phylum. In another embodiment, the sucrose synthase is derived from the species Acidithiobacillus caldus, Nitrosomonas europaea, Denitrovibrio acetiphilus, Thermosynechococcus elongatus, Oryza sativa, Arabidopsis thaliana, or Coffea arabica. In another embodiment, the sucrose synthase is an engineered sucrose synthase with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to a sucrose synthase derived from the species Acidithiobacillus caldus, Nitrosomonas europaea. Denitrovibrio acetiphilus, Thermosynechococcus elongatus, Oryza sativa, Arabidopsis thaliana, or Coffea arabica.

In one embodiment the B12GT is any polypeptide that can glycosylate the C2′ of the 13-O-glucose and/or 19-O-glucose of a steviol glycoside substrate. In another embodiment, the B12GT is derived from an organism from the Plantae kingdom. In another embodiment, the B12GT is derived from an organism from the Angiosperm clade. In another embodiment, the B12GT is derived from the species Hordeum vulgare, Oryza sativa, Solanum lycopersicum, Lycium barbarim, Solanum tuberosum, or Stevia rebaudiana. In another embodiment, the B12GT is an engineered B12GT with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to a B12GT derived from the species Hordeum vulgare, Oryza sativa, Solanum lycopersicutm, Lycium barbarum, Solanum tuberosum, or Slevia rebaudiana.

In some embodiments, the B13GT, B12GT and/or sucrose synthase polypeptides are prepared by expression in a host microorganism. Suitable host microorganisms include, but are not limited to, E. coli, Saccharomyces sp., Aspergillus sp., Pichia sp., Bacillus sp. In a particular embodiment, the glycosyltransferase and sucrose synthase are expressed in E. coli. In another embodiment, the glycosyltransferase and sucrose synthase are expressed in Pichia pastoris. In another embodiment, the B13GT, B12GT and/or sucrose synthase polypeptides are prepared by cell-free expression.

The B13GT, B12GT and sucrose synthase polypeptides can be provided in any suitable form, including free, immobilized or as a whole cell system. The degree of purity of the polypeptides may vary, e.g., they may be provided as a crude, semi-purified or purified enzyme preparation(s). In one embodiment, the B13GT, B12GT and/or SuSy polypeptide is free. In another embodiment, the B13GT, B12GT and/or SuSy polypeptide is immobilized to a solid support, for example on an inorganic or organic support. In some embodiments, the solid support is derivatized cellulose, glass, ceramic, methacrylate, styrene, acrylic, a metal oxide, or a membrane. In some embodiments, the B13GT, B12GT and/or SuSy polypeptide is immobilized to the solid support by covalent attachment, adsorption, cross-linking, entrapment, or encapsulation.

In yet another embodiment, the B13GT, B12GT and/or SuSy polypeptide is provided in the form of a whole cell system, for example as a living fermentative microbial cell, or as dead and stabilized microbial cell, or in the form of a cell lysate.

The steviol glycoside component(s) of the starting composition serve as a substrate(s) for the production of the target steviol glycoside(s), as described herein. The target steviol glycosides differ chemically from their corresponding substrate steviol glycoside(s) by the addition of one or more glucose units.

The starting steviol glycoside composition can contain at least one substrate steviol glycoside. In one embodiment, the substrate steviol glycoside is selected from the group consisting of steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside E2, rebaudioside H, rebaudioside L, rebaudioside K, rebaudioside J, rebaudioside M, rebaudioside AM, rebaudioside D, rebaudioside N, rebaudioside O, rebaudioside Q, an isomer thereof, a synthetic steviol glycoside or combinations thereof. In another embodiment the starting steviol glycoside composition comprises stevioside and Reb A. In another embodiment the starting steviol glycoside composition comprises stevioside. In yet another embodiment the starting steviol glycoside composition comprises Reb A.

The starting steviol glycoside composition may be synthetic or purified (partially or entirely), commercially available or prepared. One example of a starting composition useful in the method of the present disclosure is an extract obtained from purification of Stevia rebaudiana plant material (e.g. leaves). Another example of a starting composition is a commercially available stevia extract brought into solution with a solvent. Yet another example of a starting composition is a commercially available mixture of steviol glycosides brought into solution with a solvent. Other suitable starting compositions include by-products of processes to isolate and purify steviol glycosides.

In one embodiment, the starting composition comprises a purified substrate steviol glycoside. For example, the starting composition may comprise greater than 50%, greater than 60%, greater than 70%, greater than 80%, greater than 85%, greater than 90%, greater than 91%, greater than 92%, greater than 93%, greater than 94%, greater than 95%, greater than 96%, greater than 97%, greater than 98%, greater than 99%, or greater than 99.6% of one or more substrate steviol glycosides by weight on an anhydrous basis.

In another embodiment, the starting composition comprises a partially purified substrate steviol glycoside composition. For example, the starting composition contains greater than 0.5%, greater than 1%, greater than 2%, greater than 3%, greater than 4%, greater than 5%, greater than 10%, greater than 20%, greater than 30%, greater than 40%, or greater than 50%, of one or more substrate steviol glycosides by weight on an anhydrous basis.

In another embodiment, the substrate steviol glycoside is purified rebaudioside A, or isomers thereof. In a particular embodiment, the substrate steviol glycoside contains greater than 99% rebaudioside A, or isomers thereof, by weight on an anhydrous basis. In another embodiment, the substrate steviol glycoside comprises partially purified rebaudioside A. In a particular embodiment, the substrate steviol glycoside contains greater than 1%, 5%, 10%, 20%, 30%, 40%, 50%, 60%, 70%, 80% or 90% rebaudioside A by weight on an anhydrous basis.

In yet another embodiment, the substrate steviol glycoside comprises purified stevioside, or isomers thereof. In a particular embodiment, the substrate steviol glycoside contains greater than 99% stevioside, or isomers thereof, by weight on an anhydrous basis. In another embodiment, the substrate steviol glycoside comprises partially purified stevioside. In a particular embodiment, the substrate steviol glycoside contains greater than 10%, 20%, 30%, 40%, 50%, 60%, 70%, 80% or 90% stevioside by weight on an anhydrous basis.

In yet another embodiment, the substrate steviol glycoside is a combination of stevioside and rebaudioside A. In a particular embodiment, the substrate steviol glycoside contains greater than 5% stevioside and greater than 5% Reb A, greater than 10% stevioside and greater than 10% Reb A, greater than 20% stevioside and greater than 20% Reb A, greater than 30% stevioside and greater than 30% Reb A, greater than 40% stevioside and greater than 40% Reb A, greater than 45% stevioside and greater than 45% Reb A, greater than 40% stevioside and greater than 50% Reb A, greater than 30% stevioside and greater than 60% Reb A, greater than 20% stevioside and greater than 70% Reb A, greater than 10% stevioside and greater than 80% Reb A, greater than 5% stevioside and greater than 90% Reb A, greater than 50% stevioside and greater than 40% Reb A, greater than 60% stevioside and greater than 30% Reb A, greater than 70% stevioside and greater than 20% Reb A, greater than 80% stevioside and greater than 10% Reb A, or greater than 90% stevioside and greater than 5% Reb A by weight on an anhydrous basis.

In still another embodiment, the substrate steviol glycoside is derived from stevia leaf extract. In one embodiment, RA50, stevia leaf extract purified to contain greater than 50% Reb A, is used as the steviol glycoside substrate. In one embodiment, RA50 is used at a concentration between 1 mg/ml and 800 mg/ml. In another embodiment, RA50 is used at a concentration of 100 mg/ml. In another embodiment, RA60, stevia leaf extract purified to contain greater than 60% Reb A, is used as the steviol glycoside substrate. In another embodiment, RA60 is used at a concentration between 1 mg/mI and 800 mg/ml. In another embodiment, RA60 is used at a concentration of 100 mg/ml. In another embodiment, RS90, stevia leaf extract purified to contain greater than 90% stevioside, is used as the steviol glycoside substrate. In another embodiment, RS99, stevia leaf extract purified to contain greater than 99% stevioside, is used as the steviol glycoside substrate. In another embodiment, RS90 is used at a concentration between 1 mg/ml and 800 mg/ml. In another embodiment, RS90 is used at a concentration of 100 mg/ml.

The reaction can be carried out with a nucleotide cofactor that can be converted to an NDP-glucose by sucrose synthase. In some embodiments, the nucleotide can be ADP, GDP, UDP, CDP, or TDP. In some embodiments, the nucleotide can be a non-UDP nucleotide (i.e. ADP, GDP, CDP, or TDP). In another embodiment, the nucleotide is ADP. In a particular embodiment, the reaction can be carried out with ADP at a concentration between 0.01 mM and 10 mM, such as, for example, between 0.01 mM and 0.05 mM, between 0.05 mM and 0.1 mM, between 0.1 mM and 0.5 mM, between 0.5 mM and 1 mM, between 1 mM and 5 mM, or between 5 mM and 10 mM. In a particular embodiment, ADP is used at a concentration of0.5 mM.

The reaction can be carried out with a sucrose concentration between 10 mM and 2M, such as, for example, greater than 10 mM, greater than 50 mM, greater than 100 mM, greater than 250 mM, greater than 500 mM, greater than 1 M, greater than 1.5 M and greater than 2 M. In a particular embodiment, sucrose is used at a concentration of 450 mM.

In one embodiment, the reaction is run at any temperature. In another embodiment, the reaction is run at a temperature between 10 °C and 80 °C. Such as, for example, between 10 °C to 20 °C, between 20 °C to 30 °C, between 30 °C to 40 °C, between 40 °C to 50 °C, between 50 °C to 60 °C, between 60 °C to 70 °C, between 70 °C to 80 °C or 80 °C. In a particular embodiment, the one-pot reaction is carried out at 60 °C.

The reaction medium for conversion is generally aqueous, e.g., purified water, buffer, or a combination thereof. In a particular embodiment, the reaction medium is a buffer. Suitable buffers include, but are not limited to, acetate buffer, citrate buffer, HEPES, and phosphate buffer. In a particular embodiment, the reaction medium is phosphate buffer. The reaction medium can have a pH between 4 and 10. In a particular embodiment, the reaction medium has a pH of 6. The reaction medium can also be, alternatively, an organic solvent.

The step of contacting the starting composition with the glycosyltransferase and sucrose synthase polypeptides can be carried out in a duration of time between 1 hour and 1 week, such as, for example, between 30 minutes and 1 hours, between 1 hour and 4 hours, between 4 hours and 6 hours, between 6 hours and 12 hours, between 12 hours and 24 hours, between 1 day and 2 days, between 2 days and 3 days, 3 days and 4 days, between 4 days and 5 days, between 6 days and 7 days. In a particular embodiment, the reaction is carried out for 6 hours.

The reaction can be monitored by suitable methods including, but not limited to, high-performance liquid chromatography (HPLC), liquid chromatography-mass spectrometry (LCMS), thin layer chromatography (TLC), infrared spectrometry (IR), or nuclear magnetic resonance (NMR).

The target steviol glycoside can be any steviol glycoside. In one embodiment, the target steviol glycoside is steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside E2, rebaudioside H, rebaudioside L, rebaudioside K, rebaudioside J, rebaudioside M, rebaudioside AM, rebaudioside D, rebaudioside N, rebaudioside O, rebaudioside Q, a rebaudioside with 7 covalently attached glucose units (e.g. rebaudioside M plus 1 glucose unit), a synthetic steviol glycoside, an isomer thereof, and/or a steviol glycoside composition. In another embodiment, the target steviol glycoside is rebaudioside I, or isomers thereof. In another embodiment, the target steviol glycoside is rebaudioside M, or isomers thereof. In another embodiment, the target steviol glycoside is rebaudioside AM, or isomers thereof. In another embodiment, the target steviol glycoside is rebaudioside E2, or isomers thereof. In still another embodiment, the target steviol glycosides are Reb I, Reb AM, Reb D and Reb M.

In one embodiment, the conversion of the starting steviol glycoside composition to Reb I is at least 2% complete, as determined by any of the methods mentioned above. In a particular embodiment, the conversion of the starting steviol glycoside composition to Reb I is at least 10% complete, at least 20% complete, at least 30% complete, at least 40% complete, at least 50% complete, at least 60% complete, at least 70% complete, at least 80% complete or at least 90% complete. In a particular embodiment, the conversion of starting steviol glycoside composition to Reb I is at least 95% complete. In some embodiments, wherein at least 5%, 10%, 20%, 30%, 40%, 50%, 60%, 70%, 80%, or 90% of the starting steviol glycoside composition is converted to Reb I.

In one embodiment, the conversion of the starting steviol glycoside composition to Reb M is at least 2% complete, as determined by any of the methods mentioned above. In a particular embodiment, the conversion of the starting steviol glycoside composition to Reb M is at least 10% complete, at least 20% complete, at least 30% complete, at least 40% complete, at least 50% complete, at least 60% complete, at least 70% complete, at least 80% complete or at least 90% complete. In a particular embodiment, the conversion of the starting steviol glycoside composition to Reb M is at least 95% complete. In some embodiments, wherein at least 5%, 10%, 20%, 30%, 40%, 50%, 60%, 70%, 80%, or 90% of the starting steviol glycoside composition is converted to Reb M.

In one embodiment, the conversion of the starting steviol glycoside composition to Reb AM is at least 2% complete, as determined by any of the methods mentioned above. In a particular embodiment, the conversion of the starting steviol glycoside composition to Reb AM is at least 10% complete, at least 20% complete, at least 30% complete, at least 40% complete, at least 50% complete, at least 60% complete, at least 70% complete, at least 80% complete or at least 90% complete. In a particular embodiment, the conversion of the starting steviol glycoside composition to Reb AM is at least 95% complete. In some embodiments, wherein at least 5%, 10%, 20%, 30%, 40%, 50%, 60%, 70%, 80%, or 90% of the starting steviol glycoside composition is converted to Reb AM.

In one embodiment, the conversion of the starting steviol glycoside composition to Reb E2 is at least 2% complete, as determined by any of the methods mentioned above. In a particular embodiment, the conversion of the starting steviol glycoside composition to Reb E2 is at least 10% complete, at least 20% complete, at least 30% complete, at least 40% complete, at least 50% complete, at least 60% complete, at least 70% complete, at least 80% complete or at least 90% complete. In a particular embodiment, the conversion of the starting steviol glycoside composition to reb E2 is at least 95% complete. In some embodiments, wherein at least 5%, 10%, 20%, 30%, 40%, 50%, 60%, 70%, 80%, or 90% of the starting steviol glycoside composition is converted to Reb E2.

In one embodiment, the conversion of the starting steviol glycoside composition to Reb I, Reb AM, Reb D and Reb M is at least 2% complete, as determined by any of the methods mentioned above. In a particular embodiment, the conversion of the starting steviol glycoside composition to Reb I, Reb AM, Reb D and Reb M is at least 10% complete, at least 20% complete, at least 30% complete, at least 40% complete, at least 50% complete, at least 60% complete, at least 70% complete, at least 80% complete or at least 90% complete. In a particular embodiment, the conversion of the starting steviol glycoside composition to Reb E2 is at least 95% complete. In some embodiments, wherein at least 5%, 10%, 20%, 30%, 40%, 50%, 60%, 70%, 80%, or 90% of the starting steviol glycoside composition is converted to Reb I, Reb AM, Reb D and Reb M.

The target steviol glycoside(s) can be in any polymorphic or amorphous form, including hydrates, solvates, anhydrous or combinations thereof.

Optionally, the method of the present disclosure further comprises separating the target steviol glycoside from the target composition. The target steviol glycoside(s) can be separated by any suitable method, such as, for example, crystallization, separation by membranes, centrifugation, extraction, chromatographic separation or a combination of such methods.

In one embodiment, the separation of target steviol glycosides produces a composition comprising greater than 80% by weight of the target steviol glycoside(s) on an anhydrous basis, i.e., a highly purified steviol glycoside composition. In another embodiment, separation produces a composition comprising greater than 0.5%, greater than 1%, greater than 2%, greater than 3%, greater than 4%, greater than 5%, greater than 10%, greater than 20%, greater than 30%, greater than 40%, greater than 50%, greater than 60%, greater than 70%, greater than 80%, greater than 85%, greater than 90%, greater than 91%, greater than 92%, greater than 93%, greater than 94%, greater than 95%, greater than 96%, greater than 97%, greater than 98%, greater than 99%, or greater than 99.6% by weight of the target steviol glycosides. In particular embodiments, the composition comprises greater than 95% by weight of the target steviol glycoside(s) on an anhydrous basis.

Purified target steviol glycosides can be used in consumable products as a sweetener. Suitable consumer products include, but are not limited to, food, beverages, pharmaceutical compositions, tobacco products, nutraceutical compositions, oral hygiene compositions, and cosmetic compositions.

Plasmids containing nucleic acids encoding enzymes having SEQ ID NOs: 2-517 are described in Table 1 below.

TABLE 1
Designed B13GTs

| SEQ | Plasmid | Amino |
|---|---|---|
| ID | ID | Acid ID |
| 2 | pA35354 | AA29161 |
| 3 | pA35355 | AA29162 |
| 4 | pA35356 | AA29163 |
| 5 | pA35357 | AA29164 |
| 6 | pA35358 | AA29165 |
| 7 | pA35359 | AA29166 |
| 8 | pA35360 | AA29167 |
| 9 | pA35361 | AA29168 |
| 10 | pA35362 | AA29169 |
| 11 | pA35363 | AA29170 |
| 12 | pA35364 | AA29171 |
| 13 | pA35365 | AA29172 |
| 14 | pA35366 | AA29173 |
| 15 | pA35367 | AA29174 |
| 16 | pA35368 | AA29175 |
| 17 | pA35369 | AA29176 |
| 18 | pA35370 | AA29177 |
| 19 | pA35371 | AA29178 |
| 20 | pA35372 | AA29179 |
| 21 | pA35373 | AA29180 |
| 22 | pA35374 | AA29181 |
| 23 | pA35375 | AA29182 |
| 24 | pA35376 | AA29183 |
| 25 | pA35377 | AA29184 |
| 26 | pA35378 | AA29185 |
| 27 | pA35379 | AA29186 |
| 28 | pA35380 | AA29187 |
| 29 | pA35381 | AA29188 |
| 30 | pA35382 | AA29189 |
| 31 | pA35383 | AA29190 |
| 32 | pA35384 | AA29191 |
| 33 | pA35385 | AA29192 |
| 34 | pA35386 | AA29193 |
| 35 | pA35387 | AA29194 |
| 36 | pA35388 | AA29195 |
| 37 | pA35389 | AA29196 |
| 38 | pA35390 | AA29197 |
| 39 | pA35391 | AA29198 |
| 40 | pA35392 | AA29199 |
| 41 | pA35393 | AA29200 |
| 42 | pA35394 | AA29201 |
| 43 | pA35395 | AA29202 |
| 44 | pA35396 | AA29203 |
| 45 | pA35397 | AA29204 |
| 46 | pA35398 | AA29205 |
| 47 | pA35399 | AA29206 |
| 48 | pA35400 | AA29207 |
| 49 | pA35401 | AA29208 |
| 50 | pA35402 | AA29209 |
| 51 | pA35403 | AA29210 |
| 52 | pA35404 | AA29211 |
| 53 | pA35405 | AA29212 |
| 54 | pA35406 | AA29213 |
| 55 | pA49732 | AA35898 |
| 56 | pA49716 | AA35899 |
| 57 | pA49714 | AA35900 |
| 58 | pA49719 | AA35901 |
| 59 | pA49738 | AA35902 |
| 60 | pA49780 | AA35903 |
| 61 | pA49721 | AA35904 |
| 62 | pA49706 | AA35905 |
| 63 | pA49729 | AA35906 |
| 64 | pA49742 | AA35907 |
| 65 | pA49772 | AA35908 |
| 66 | pA49752 | AA35909 |
| 67 | pA49727 | AA35910 |
| 68 | pA49756 | AA35911 |
| 69 | pA49768 | AA35912 |
| 70 | pA49778 | AA35913 |
| 71 | pA49782 | AA35914 |
| 72 | pA49753 | AA35915 |
| 73 | pA49771 | AA35916 |
| 74 | pA49745 | AA35917 |
| 75 | pA49759 | AA35918 |
| 76 | pA49758 | AA35919 |
| 77 | pA49776 | AA35920 |
| 78 | pA49731 | AA35921 |
| 79 | pA49707 | AA35922 |
| 80 | pA49724 | AA35923 |
| 81 | pA49767 | AA35924 |
| 82 | pA49739 | AA35925 |
| 83 | pA49757 | AA35926 |
| 84 | pA49730 | AA35927 |
| 85 | pA49762 | AA35928 |
| 86 | pA49769 | AA35929 |
| 87 | pA49718 | AA35930 |
| 88 | pA49741 | AA35931 |
| 89 | pA49737 | AA35932 |
| 90 | pA49749 | AA35933 |
| 91 | pA49704 | AA35934 |
| 92 | pA49715 | AA35935 |
| 93 | pA49734 | AA35936 |
| 94 | pA49747 | AA35937 |
| 95 | pA49766 | AA35938 |
| 96 | pA49735 | AA35939 |
| 97 | pA49709 | AA35940 |
| 98 | pA49712 | AA35941 |
| 99 | pA49726 | AA35942 |
| 100 | pA49708 | AA35943 |
| 101 | pA49743 | AA35944 |
| 102 | pA49751 | AA35945 |
| 103 | pA49761 | AA35946 |
| 104 | pA49783 | AA35947 |
| 105 | pA49705 | AA35948 |
| 106 | pA49750 | AA35949 |
| 107 | pA49736 | AA35950 |
| 108 | pA49775 | AA35951 |
| 109 | pA49733 | AA35952 |
| 110 | pA49728 | AA35953 |
| 111 | pA49770 | AA35954 |
| 112 | pA49774 | AA35955 |
| 113 | pA49764 | AA35956 |
| 114 | pA49779 | AA35957 |
| 115 | pA49722 | AA35958 |
| 116 | pA49720 | AA35959 |
| 117 | pA49725 | AA35960 |
| 118 | pA49713 | AA35961 |
| 119 | pA49760 | AA35962 |
| 120 | pA49711 | AA35963 |
| 121 | pA49777 | AA35964 |
| 122 | pA49781 | AA35965 |
| 123 | pA49763 | AA35966 |
| 124 | pA49748 | AA35967 |
| 125 | pA49740 | AA35968 |
| 126 | pA49717 | AA35969 |
| 127 | pA49773 | AA35970 |
| 128 | pA49746 | AA35971 |
| 129 | pA49710 | AA35972 |
| 130 | pA49744 | AA35973 |
| 131 | pA49754 | AA35974 |
| 132 | pA49755 | AA35975 |
| 133 | pA49723 | AA35976 |
| 134 | pA49765 | AA35977 |
| 135 | pA51787 | AA40159 |
| 136 | pA51788 | AA40160 |
| 137 | pA51789 | AA40161 |
| 138 | pA51790 | AA40162 |
| 139 | pA51791 | AA40163 |
| 140 | pA51792 | AA40164 |
| 141 | pA51793 | AA40165 |
| 142 | pA51794 | AA40166 |
| 143 | pA51795 | AA40167 |
| 144 | pA51796 | AA40168 |
| 145 | pA51797 | AA40169 |
| 146 | pA51798 | AA40170 |
| 147 | pA51799 | AA40171 |
| 148 | pA51800 | AA40172 |
| 149 | pA51801 | AA40173 |
| 150 | pA51802 | AA40174 |
| 151 | pA51803 | AA40175 |
| 152 | pA51804 | AA40176 |
| 153 | pA51805 | AA40177 |
| 154 | pA51806 | AA40178 |
| 155 | pA51807 | AA40179 |
| 156 | pA51808 | AA40180 |
| 157 | pA51809 | AA40181 |
| 158 | pA51810 | AA40182 |
| 159 | pA51811 | AA40183 |
| 160 | pA51812 | AA40184 |
| 161 | pA51813 | AA40185 |
| 162 | pA51814 | AA40186 |
| 163 | pA51815 | AA40187 |
| 164 | pA51816 | AA40188 |
| 165 | pA51817 | AA40189 |
| 166 | pA51818 | AA40190 |
| 167 | pA51819 | AA40191 |
| 168 | pA51820 | AA40192 |
| 169 | pA51821 | AA40193 |
| 170 | pA51822 | AA40194 |
| 171 | pA51823 | AA40195 |
| 172 | pA51824 | AA40196 |
| 173 | pA51825 | AA40197 |
| 174 | pA51826 | AA40198 |
| 175 | pA51827 | AA40199 |
| 176 | pA51828 | AA40200 |
| 177 | pA51829 | AA40201 |
| 178 | pA51830 | AA40202 |
| 179 | pA51831 | AA40203 |
| 180 | pA51832 | AA40204 |
| 181 | pA51833 | AA40205 |
| 182 | pA51834 | AA40206 |
| 183 | pA51835 | AA40207 |
| 184 | pA51836 | AA40208 |
| 185 | pA51837 | AA40209 |
| 186 | pA51838 | AA40210 |
| 187 | pA51839 | AA40211 |
| 188 | pA51840 | AA40212 |
| 189 | pA51841 | AA40213 |
| 190 | pA51842 | AA40214 |
| 191 | pA51843 | AA40215 |
| 192 | pA51844 | AA40216 |
| 193 | pA51845 | AA40217 |
| 194 | pA51846 | AA40218 |
| 195 | pA51847 | AA40219 |
| 196 | pA51848 | AA40220 |
| 197 | pA51849 | AA40221 |
| 198 | pA51850 | AA40222 |
| 199 | pA51851 | AA40223 |
| 200 | pA51852 | AA40224 |
| 201 | pA51853 | AA40225 |
| 202 | pA51854 | AA40226 |
| 203 | pA51855 | AA40227 |
| 204 | pA51856 | AA40228 |
| 205 | pA51857 | AA40229 |
| 206 | pA51858 | AA40230 |
| 207 | pA51859 | AA40231 |
| 208 | pA51860 | AA40232 |
| 209 | pA51861 | AA40233 |
| 210 | pA51862 | AA40234 |
| 211 | pA51863 | AA40235 |
| 212 | pA51864 | AA40236 |
| 213 | pA51865 | AA40237 |
| 214 | pA51866 | AA40238 |
| 215 | pA51867 | AA40239 |
| 216 | pA51868 | AA40240 |
| 217 | pA51869 | AA40241 |
| 218 | pA51870 | AA40242 |
| 219 | pA51871 | AA40243 |
| 220 | pA51872 | AA40244 |
| 221 | pA51873 | AA40245 |
| 222 | pA51874 | AA40246 |
| 223 | pA51875 | AA40247 |
| 224 | pA51876 | AA40248 |
| 225 | pA51877 | AA40249 |
| 226 | pA51878 | AA40250 |
| 227 | pA51879 | AA40251 |
| 228 | pA51880 | AA40252 |
| 229 | pA51881 | AA40253 |
| 230 | pA51882 | AA40254 |
| 231 | pA52063 | AA40415 |
| 232 | pA52064 | AA40416 |
| 233 | pA52065 | AA40417 |
| 234 | pA52066 | AA40418 |
| 235 | pA52067 | AA40419 |
| 236 | pA52068 | AA40420 |
| 237 | pA52069 | AA40421 |
| 238 | pA52070 | AA40422 |
| 239 | pA52071 | AA40423 |
| 240 | pA52072 | AA40424 |
| 241 | pA52073 | AA40425 |
| 242 | pA52074 | AA40426 |
| 243 | pA52075 | AA40427 |
| 244 | pA52076 | AA40428 |
| 245 | pA52077 | AA40429 |
| 246 | pA52078 | AA40430 |
| 247 | pA52079 | AA40431 |
| 248 | pA52080 | AA40432 |
| 249 | pA52081 | AA40433 |
| 250 | pA52082 | AA40434 |
| 251 | pA52083 | AA40435 |
| 252 | pA52084 | AA40436 |
| 253 | pA52085 | AA40437 |
| 254 | pA52086 | AA40438 |
| 255 | pA52087 | AA40439 |
| 256 | pA52132 | AA40547 |
| 257 | pA52133 | AA40548 |
| 258 | pA52134 | AA40549 |
| 259 | pA52135 | AA40550 |
| 260 | pA52136 | AA40551 |
| 261 | pA52137 | AA40552 |
| 262 | pA52138 | AA40553 |
| 263 | pA52139 | AA40554 |
| 264 | pA52140 | AA40555 |
| 265 | pA52141 | AA40556 |
| 266 | pA52142 | AA40557 |
| 267 | pA52143 | AA40558 |
| 268 | pA52144 | AA40559 |
| 269 | pA52145 | AA40560 |
| 270 | pA52146 | AA40561 |
| 271 | pA52147 | AA40562 |
| 272 | pA52148 | AA40563 |
| 273 | pA52149 | AA40564 |
| 274 | pA52150 | AA40565 |
| 275 | pA52151 | AA40566 |
| 276 | pA52152 | AA40567 |
| 277 | pA52153 | AA40568 |
| 278 | pA52154 | AA40569 |
| 279 | pA52155 | AA40570 |
| 280 | pA52156 | AA40571 |
| 281 | pA52157 | AA40572 |
| 282 | pA52158 | AA40573 |
| 283 | pA52159 | AA40574 |
| 284 | pA52160 | AA40575 |
| 285 | pA52161 | AA40576 |
| 286 | pA52162 | AA40577 |
| 287 | pA52163 | AA40578 |
| 288 | pA52164 | AA40579 |
| 289 | pA52165 | AA40580 |
| 290 | pA52166 | AA40581 |
| 291 | pA52167 | AA40582 |
| 292 | pA52168 | AA40583 |
| 293 | pA52169 | AA40584 |
| 294 | pA52170 | AA40585 |
| 295 | pA52171 | AA40586 |
| 296 | pA52172 | AA40587 |
| 297 | pA52173 | AA40588 |
| 298 | pA52174 | AA40589 |
| 299 | pA52175 | AA40590 |
| 300 | pA52176 | AA40591 |
| 301 | pA52177 | AA40592 |
| 302 | pA52178 | AA40593 |
| 303 | pA52179 | AA40594 |
| 304 | pA52180 | AA40595 |
| 305 | pA52181 | AA40596 |
| 306 | pA52182 | AA40597 |
| 307 | pA52183 | AA40598 |
| 308 | pA52184 | AA40599 |
| 309 | pA52186 | AA40601 |
| 310 | pA52187 | AA40602 |
| 311 | pA52188 | AA40603 |
| 312 | pA52189 | AA40604 |
| 313 | pA52190 | AA40605 |
| 314 | pA52191 | AA40606 |
| 315 | pA52192 | AA40607 |
| 316 | pA52193 | AA40608 |
| 317 | pA52194 | AA40609 |
| 318 | pA52195 | AA40610 |
| 319 | pA52196 | AA40611 |
| 320 | pA52197 | AA40612 |
| 321 | pA52198 | AA40613 |
| 322 | pA52199 | AA40614 |
| 323 | pA52200 | AA40615 |
| 324 | pA52201 | AA40616 |
| 325 | pA53133 | AA41183 |
| 326 | pA53134 | AA41184 |
| 327 | pA53135 | AA41185 |
| 328 | pA53136 | AA41186 |
| 329 | pA53137 | AA41187 |
| 330 | pA53138 | AA41188 |
| 331 | pA53139 | AA41189 |
| 332 | pA53140 | AA41190 |
| 333 | pA53141 | AA41191 |
| 334 | pA53142 | AA41192 |
| 335 | pA53143 | AA41193 |
| 336 | pA53144 | AA41194 |
| 337 | pA53145 | AA41195 |
| 338 | pA53146 | AA41196 |
| 339 | pA53147 | AA41197 |
| 340 | pA53148 | AA41198 |
| 341 | pA53149 | AA41199 |
| 342 | pA53150 | AA41200 |
| 343 | pA53151 | AA41201 |
| 344 | pA53152 | AA41202 |
| 345 | pA53153 | AA41203 |
| 346 | pA53154 | AA41204 |
| 347 | pA53155 | AA41205 |
| 348 | pA53156 | AA41206 |
| 349 | pA53157 | AA41207 |
| 350 | pA53158 | AA41208 |
| 351 | pA53159 | AA41209 |
| 352 | pA53160 | AA41210 |
| 353 | pA53161 | AA41211 |
| 354 | pA53162 | AA41212 |
| 355 | pA53163 | AA41213 |
| 356 | pA53164 | AA41214 |
| 357 | pA53165 | AA41215 |
| 358 | pA53166 | AA41216 |
| 359 | pA53167 | AA41217 |
| 360 | pA53168 | AA41218 |
| 361 | pA53169 | AA41219 |
| 362 | pA53170 | AA41220 |
| 363 | pA53171 | AA41221 |
| 364 | pA53172 | AA41222 |
| 365 | pA53173 | AA41223 |
| 366 | pA53174 | AA41224 |
| 367 | pA53175 | AA41225 |
| 368 | pA53176 | AA41226 |
| 369 | pA53177 | AA41227 |
| 370 | pA53178 | AA41228 |
| 371 | pA53179 | AA41229 |
| 372 | pA53180 | AA41230 |
| 373 | pA53181 | AA41231 |
| 374 | pA53182 | AA41232 |
| 375 | pA53183 | AA41233 |
| 376 | pA53184 | AA41234 |
| 377 | pA53185 | AA41235 |
| 378 | pA53186 | AA41236 |
| 379 | pA53187 | AA41237 |
| 380 | pA53188 | AA41238 |
| 381 | pA53189 | AA41239 |
| 382 | pA53190 | AA41240 |
| 383 | pA53191 | AA41241 |
| 384 | pA53192 | AA41242 |
| 385 | pA53193 | AA41243 |
| 386 | pA53194 | AA41244 |
| 387 | pA53195 | AA41245 |
| 388 | pA53196 | AA41246 |
| 389 | pA53197 | AA41247 |
| 390 | pA53198 | AA41248 |
| 391 | pA53199 | AA41249 |
| 392 | pA53200 | AA41250 |
| 393 | pA53201 | AA41251 |
| 394 | pA53202 | AA41252 |
| 395 | pA53203 | AA41253 |
| 396 | pA53204 | AA41254 |
| 397 | pA53205 | AA41255 |
| 398 | pA53206 | AA41256 |
| 399 | pA53207 | AA41257 |
| 400 | pA53208 | AA41258 |
| 401 | pA53209 | AA41259 |
| 402 | pA53210 | AA41260 |
| 403 | pA53211 | AA41261 |
| 404 | pA53212 | AA41262 |
| 405 | pA53213 | AA41263 |
| 406 | pA53214 | AA41264 |
| 407 | pA53215 | AA41265 |
| 408 | pA53216 | AA41266 |
| 409 | pA53217 | AA41267 |
| 410 | pA53218 | AA41268 |
| 411 | pA53219 | AA41269 |
| 412 | pA53220 | AA41270 |
| 413 | pA53221 | AA41271 |
| 414 | pA53222 | AA41272 |
| 415 | pA53223 | AA41273 |
| 416 | pA53224 | AA41274 |
| 417 | pA53225 | AA41275 |
| 418 | pA53226 | AA41276 |
| 419 | pA53227 | AA41277 |
| 420 | pA53228 | AA41278 |
| 421 | pA53229 | AA41279 |
| 422 | pA53230 | AA41280 |
| 423 | pA53231 | AA41281 |
| 424 | pA53232 | AA41282 |
| 425 | pA53233 | AA41283 |
| 426 | pA53235 | AA41285 |
| 427 | pA53242 | AA41292 |
| 428 | pA53243 | AA41293 |
| 429 | pA53244 | AA41294 |
| 430 | pA53245 | AA41295 |
| 431 | pA53246 | AA41296 |
| 432 | pA53247 | AA41297 |
| 433 | pA53248 | AA41298 |
| 434 | pA53249 | AA41299 |
| 435 | pA53250 | AA41300 |
| 436 | pA53251 | AA41301 |
| 437 | pA53252 | AA41302 |
| 438 | pA53253 | AA41303 |
| 439 | pA53254 | AA41304 |
| 440 | pA53255 | AA41305 |
| 441 | pA53256 | AA41306 |
| 442 | pA53257 | AA41307 |
| 443 | pA53258 | AA41308 |
| 444 | pA53259 | AA41309 |
| 445 | pA53260 | AA41310 |
| 446 | pA53261 | AA41311 |
| 447 | pA53262 | AA41312 |
| 448 | pA53263 | AA41313 |
| 449 | pA53264 | AA41314 |
| 450 | pA53265 | AA41315 |
| 451 | pA53263 | AA41316 |
| 452 | pA53267 | AA41317 |
| 453 | pA53268 | AA41318 |
| 454 | pA53269 | AA41319 |
| 455 | pA53270 | AA41320 |
| 456 | pA53271 | AA41321 |
| 457 | pA53272 | AA41322 |
| 458 | pA53273 | AA41323 |
| 459 | pA53274 | AA41324 |
| 460 | pA53275 | AA41325 |
| 461 | pA53276 | AA41326 |
| 462 | pA53277 | AA41327 |
| 463 | pA53278 | AA41328 |
| 464 | pA53279 | AA41329 |
| 465 | pA53280 | AA41330 |
| 466 | pA53281 | AA41331 |
| 467 | pA53282 | AA41332 |
| 468 | pA53283 | AA41333 |
| 469 | pA53284 | AA41334 |
| 470 | pA53285 | AA41335 |
| 471 | pA53286 | AA41336 |
| 472 | pA53287 | AA41337 |
| 473 | pA53288 | AA41338 |
| 474 | pA53289 | AA41339 |
| 475 | pA53290 | AA41340 |
| 476 | pA53291 | AA41341 |
| 477 | pA53292 | AA41342 |
| 478 | pA53293 | AA41343 |
| 479 | pA53294 | AA41344 |
| 480 | pA53295 | AA41345 |
| 481 | pA53296 | AA41346 |
| 482 | pA53297 | AA41347 |
| 483 | pA53298 | AA41348 |
| 484 | pA53299 | AA41349 |
| 485 | pA53300 | AA41350 |
| 486 | pA53301 | AA41351 |
| 487 | pA53302 | AA41352 |
| 488 | pA53303 | AA41353 |
| 489 | pA53304 | AA41354 |
| 490 | pA53305 | AA41355 |
| 491 | pA53306 | AA41356 |
| 492 | pA53307 | AA41357 |
| 493 | pA53308 | AA41358 |
| 494 | pA53309 | AA41359 |
| 495 | pA53310 | AA41360 |
| 496 | pA53311 | AA41361 |
| 497 | pA53312 | AA41362 |
| 498 | pA53313 | AA41363 |
| 499 | pA53314 | AA41364 |
| 500 | pA53315 | AA41365 |
| 501 | pA53316 | AA41366 |
| 502 | pA53317 | AA41367 |
| 503 | pA53318 | AA41368 |
| 504 | pA53319 | AA41369 |
| 505 | pA53320 | AA41370 |
| 506 | pA53321 | AA41371 |
| 507 | pA53322 | AA41372 |
| 508 | pA53323 | AA41373 |
| 509 | pA53324 | AA41374 |
| 510 | pA53325 | AA41375 |
| 511 | pA53326 | AA41376 |
| 512 | pA53327 | AA41377 |
| 513 | pA53328 | AA41378 |
| 514 | pA53329 | AA41379 |
| 515 | pA53330 | AA41380 |
| 516 | pA53331 | AA41381 |
| 517 | pA53332 | AA41382 |

### EXAMPLES

### Example 1: Computational Design of ADP-Glucose Dependent B13GTs

Computational designs were conducted to improve and convert the native B13GT from Stevia rebaudiana (Seq ID NO: 1) to an ADP-glycosyltransferase. Structural models of a B13GT variant of SEQ ID NO: 1 were generated and used as the starting point for computational designs. Visual inspection of the structural models identified residues H24 as the catalytic histidine, D123 which activates the histidine, and two sugar binding residues D379 and Q380 (numbering relative to SEQ ID NO: 2). Computational designs incorporating coevolutionary information were conducted to improve the B13GT. 53 of these computational designs were chosen for experimental validation (SEQ ID NOs: 2-54).

Polynucleotides encoding the amino acid sequences for the designed B13GTs were synthesized (Twist Bioscience) and inserted into the pARZ4 expression vector. The polynucleotides encoded the B13GT enzymes with an N-terminal His tag. Polynucleotides were either ordered as full-length genes or ordered as gene fragments and then assembled using Gibson assembly. The recombinant vectors were used in a heat shock method to transform E. coli HMS174(DE3) (Novagen) or T7 Express lysY/Iq (New England BioLabs), thereby preparing a recombinant microorganism.

The transformed recombinant microorganisms were inoculated to 1 ml LB-kanamycin medium, cultured by shaking at 37 °C overnight. The culture was inoculated to 5 ml TB-kanamycin medium and grown for 2 hours at 37 °C, followed by 25 °C for 1 hour. The culture was induced with 50 μl 50 mM IPTG and grown overnight. Finally, the culture was centrifuged at top-speed for 5 minutes and stored at −80 °C.

The microorganisms were dissolved in a lysis buffer (lysozyme, DNAseI, Bugbuster, 300 ml 20 mM HEPES pH 7.5, 500 mM NaCl, and 20 mM Imidazole). Two to three glass beads were added to each well and were disrupted by shaking at 25 °C and 220 rpm for 30 minutes. The disrupted liquid was centrifuged at 2200×g for 6-10 minutes. The obtained supernatant was loaded onto a Ni-NTA plate and shaken for 10 minutes at room temperature. The plate was centrifuged for 4 minutes at 100×g followed by two washes of500 μl binding buffer (300 ml 20 mM HEPES pH 7.5, 500 mM NaCl, 20 mM Imidazole) and two-minute centrifugation (500×g). The proteins were eluted with 150 μl elution buffer (15 ml 20 mM HEPES pH 7.5, 500 mM NaCl, 500 mM Imidazole) and shaken for 1 minute at 0.25 maximum shaking speed followed by centrifugation for 2 minutes at 500×g. The recovered protein was desalted into a buffer solution for enzyme activity evaluation (50 mM HEPES pH 7.5, 50 mM NaCl).

Each B13GT variant was assayed in a one-pot reaction with a B12GT and SuSy enzyme. B12GT and SuSy were used in excess such that in the absence of the B13GT variant, the starting RA50 feed was fully converted to RebD/E. The purified B13GT, B12GT and SuSy were reacted with 100 mg/ml RA50, 450 mM Sucrose, and 0.5 mM ADP in 50 mM pH 6 phosphate buffer, and 50 mM NaCl for 24 hours at 60 °C.

Steviol glycosides produced and consumed during the reaction, stevioside, Reb A, Reb D, Reb E, Reb I, Reb M and other minor steviol glycosides, were monitored by LCMS using an Agilent 6470 QQQ mass spectrometer (column: Waters ACQUITY UPLC HSS T3 Column, 100 mm×2.1 mm). The QQQ was run with multi reaction monitoring (MS/MS) to accurately quantitate steviol glycosides of interest. Several designed enzymes expressed well and were able to efficiently produce Reb M (Table 2).

TABLE 2
Purified Protein Concentration and Reb M Conversion of Successful
B13GT Design Variants Expressed in E. coli

| | Reb M | Purified |
|---|---|---|
| ID | Yield (%) | Conc (uM) |
| AA29162 | 76.9 | 12.4 |
| AA29163 | 50.2 | 16.5 |
| AA29161 | 49.2 | 49.6 |
| AA29165 | 35.1 | 75.5 |
| AA29166 | 29.3 | 12.9 |
| AA29201 | 20.7 | 58.5 |
| AA29164 | 17.9 | 39.1 |
| AA29174 | 14.4 | 48.6 |
| AA29177 | 12.9 | 39.4 |
| AA29181 | 12.1 | 20 |
| AA29190 | 10.1 | 15.7 |
| AA29172 | 9.3 | 73.2 |
| AA29178 | 8.5 | 65.4 |
| AA29180 | 8.5 | 14.8 |
| AA29204 | 7.6 | 17.2 |
| AA29179 | 7 | 30.3 |
| AA29173 | 6.9 | 25.9 |
| AA29200 | 6.9 | 49.4 |
| AA29171 | 6.8 | 42.8 |
| AA29184 | 4.9 | 36.6 |
| AA29183 | 4.6 | 22.8 |
| AA29186 | 4.3 | 36.8 |
| AA29167 | 3.4 | 10.1 |
| AA29175 | 3.4 | 28.4 |
| AA29203 | 3.4 | 88.5 |
| AA29213 | 3.2 | 28.8 |
| AA29205 | 3 | 43.1 |
| AA29187 | 2.8 | 14.7 |
| AA29202 | 2.4 | 28 |
| AA29176 | 2.2 | 19.4 |
| AA29168 | 2.1 | 11.5 |
| AA29196 | 1.9 | 35.4 |
| AA29197 | 1.7 | 27 |
| AA29169 | 1.7 | 41.6 |
| AA29185 | 1.1 | 13.2 |
| AA29194 | 1.1 | 17.8 |
| AA29192 | 1 | 13.7 |
| AA29182 | 0.8 | 52.7 |
| AA29193 | 0.8 | 30.8 |
| AA29189 | 0.7 | 22.6 |
| AA29206 | 0.7 | 27.6 |
| AA29195 | 0.6 | 37.7 |
| AA29170 | 0.5 | 65.9 |
| AA29209 | 0.2 | 11.8 |

### Example 2: Pichia pastoris Expression of Designed B13GTs and SuSys

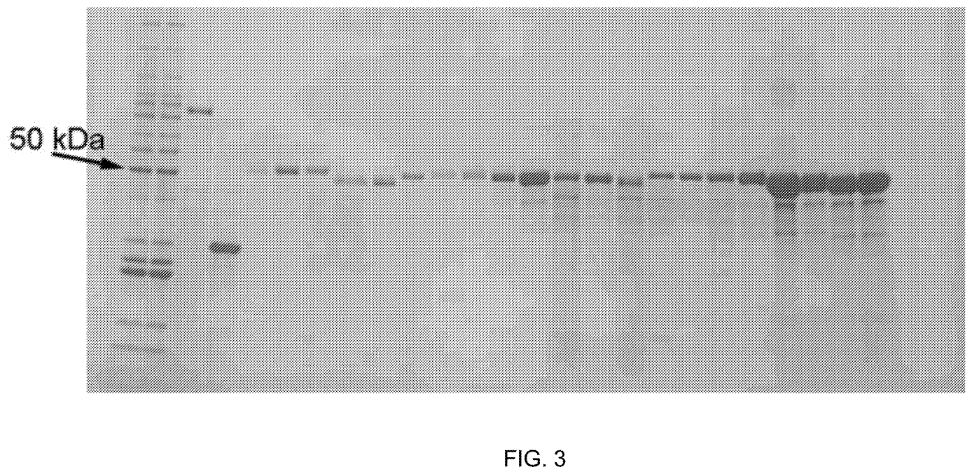

Polynucleotides of the top ten designed B13GTs from Example 1 optimized for Pichia pastoris expression were synthesized (Twist Bioscience) and inserted into a Pichia shuttle vec-tor. The polynucleotides encoded the B13GT enzymes with a C-terminal His tag instead of the N-terminal His tag used in Example 1. Two unique polynucleotides for each B13GT amino acid sequence were ordered. The vectors were transformed into a commercially available Pichia pastoris strain (ATCC). The transformed microorganisms were grown in BMGY (buff-ered glycerol complex) media and protein expression was induced by feeding of methanol. The Pichia cells were lysed with Y-PER (Yeast Protein Extraction Reagent; Thermo Scientific) and the expressed proteins were purified by immobilized metal affinity chromatography (IMAC) and desalted into desalt buffer (20 mM KPO4 pH6, 50 mM NaCl). The designed B13GTs were solubly expressed (FIG. 3) and enzyme activity was measured, similar to Example 1 (Table 3).

Polynucleotides encoding the top performing B13GTs were amplified from the Pichia shuttle vectors and integrated into the Pichia genome. Four integrated Pichia strains expressing designed B13GTs were grown in IL fermentors. The Pichia microorganisms were grown with glycerol as the main carbon source for −24 hours, and then were fed methanol for −72 hours to express the desired B13GT. The cells were collected and lysed by French press. The expressed protein was purified by immobilized metal affinity chromatography (IMAC) and dialyzed into desalt buffer (20 mM KPO4 pH6, 50 mM NaCl). All four strains successfully expressed the active B13GT in the fermentations.

TABLE 3
Purified Protein Concentration and Reb M Conversion
of Top B13GT Designs Expressed in Pichia

| | | Puri- |
|---|---|---|
| | Reb M | fied |
| | Yield | Conc |
| ID | (%) | (uM) |
| AA29162 | 97.4 | 19.9 |
| AA29162 | 94.3 | 28 |
| AA29163 | 72.8 | 12.3 |
| AA29166 | 67.4 | 25.6 |
| AA29166 | 67.3 | 22.4 |
| AA29163 | 57.3 | 17.6 |
| AA29164 | 39.1 | 15.1 |
| AA29172 | 20.3 | 8 |
| AA29177 | 17.9 | 9.5 |
| AA29174 | 17.2 | 11.8 |
| AA29161 | 16.4 | 3.6 |
| AA29177 | 16.4 | 9.6 |
| AA29165 | 16.4 | 4.9 |
| AA29174 | 14.2 | 12.1 |
| AA29172 | 8.9 | 5.4 |
| AA29161 | 6.9 | 4.2 |
| AA29201 | 3.1 | 2.3 |
| AA29201 | 1.5 | 2.5 |

### Example 3: Computational Designs of Improved B13GTs

A previously-developed protein sequence-based deep learning model was fine-tuned with the results from Example 1. The model was used to design improved B13GT enzymes. Eighty of these computational designs were chosen for experimental validation (SEQ ID NOs: 55-134). Polynucleotides encoding the B13GT enzymes with a C-terminal His tag were synthesized (Twist Bioscience). E. coli strains expressing the designed B13GTs were created as described in Example 1. The designed B13GT enzymes were expressed and purified as described in Example 1.

Each B13GT variant was assayed in a one-pot reaction with a B12GT and SuSy enzyme. B12GT and SuSy were used in excess such that in the absence of the B13GT variant, the starting RA50 feed was fully converted to RebD/E. The purified B13GT, B12GT and SuSy were reacted with 71.5 mg/mI RA50, 250 mM Sucrose, and 0.5 mM ADP in 50 mM pH 6 phosphate buffer, and 50 mM NaCl for 24 hours at 60 °C. Steviol glycosides produced and consumed during the reaction were measured as described in Example 1. The designed enzymes solubly expressed and were able to efficiently produce Reb M (Table 4).

TABLE 4
Purified Protein Concentration and Reb M Conversion
of Top B13GT Design Variants Expressed in E. coli

| | Reb | Puri- | | Reb | Puri- |
|---|---|---|---|---|---|
| | M | fied | | M | fied |
| | Yield | Conc | | Yield | Conc |
| ID | (%) | (uM) | ID | (%) | (uM) |
| AA35898 | 94.9 | 1.1 | AA35925 | 50.1 | 16.8 |
| AA35921 | 94.9 | 0.7 | AA35967 | 49.4 | 8.0 |
| AA35976 | 93.6 | 0.8 | AA35971 | 48.7 | 2.8 |
| AA35927 | 92.5 | 1.0 | AA35972 | 47.9 | 3.4 |
| AA35908 | 88.3 | 24.5 | AA35974 | 47.4 | 3.6 |
| AA35906 | 86.6 | 17.0 | AA35952 | 45.8 | 4.0 |
| AA35932 | 85.3 | 27.7 | AA35929 | 45.1 | 17.2 |
| AA35926 | 82.8 | 14.0 | AA35949 | 42.4 | 3.7 |
| AA35917 | 82.3 | 15.9 | AA35911 | 39.7 | 23.7 |
| AA35966 | 81.7 | 5.3 | AA35939 | 38.9 | 4.3 |
| AA35915 | 80.9 | 10.1 | AA35902 | 37.2 | 19.9 |
| AA35901 | 78.5 | 6.7 | AA35934 | 34 | 10.8 |
| AA35913 | 78 | 20.0 | AA35914 | 32.2 | 11.7 |
| AA35936 | 77.9 | 12.7 | AA35938 | 32 | 3.1 |
| AA35899 | 76.7 | 14.6 | AA35942 | 31.9 | 2.7 |
| AA35930 | 76.5 | 18.2 | AA35912 | 31.6 | 7.3 |
| AA35940 | 76 | 5.2 | AA35945 | 29.9 | 4.5 |
| AA35935 | 74.8 | 19.5 | AA35907 | 29.1 | 8.1 |
| AA35924 | 74.3 | 14.5 | AA35944 | 27.4 | 4.2 |
| AA35961 | 72.5 | 4.2 | AA35959 | 24.8 | 3.4 |
| AA35937 | 70.5 | 12.3 | AA35905 | 24.3 | 12.3 |
| AA35919 | 68.2 | 15.4 | AA35956 | 23.7 | 3.0 |
| AA35916 | 68 | 12.4 | AA35969 | 21 | 3.4 |
| AA35909 | 67.3 | 16.5 | AA35962 | 16 | 3.3 |
| AA35931 | 66.9 | 16.1 | AA35918 | 12.6 | 11.5 |
| AA35910 | 65.4 | 10.2 | AA35950 | 4.4 | 3.6 |
| AA35928 | 64.8 | 12.9 | AA35946 | 3.8 | 5.1 |
| AA35922 | 62 | 15.2 | AA35958 | 3.4 | 1.4 |
| AA35900 | 61.7 | 11.4 | AA35968 | 3.3 | 0.8 |
| AA35957 | 61.7 | 3.9 | AA35975 | 3.2 | 5.3 |
| AA35923 | 60.5 | 10.9 | AA35948 | 1 | 2.9 |
| AA35920 | 57.3 | 17.7 | AA35970 | 0.5 | 6.8 |
| AA35933 | 56.6 | 13.4 | AA35941 | 0.5 | 5.9 |
| AA35903 | 56.3 | 14.2 | AA35965 | 0.4 | 14.8 |
| AA35951 | 54 | 6.0 | AA35953 | 0.4 | 5.1 |
| AA35947 | 53 | 4.9 | AA35955 | 0.4 | 11.7 |
| AA35954 | 52.5 | 2.0 | AA35973 | 0.3 | 4.6 |
| AA35977 | 52.5 | 3.6 | AA35963 | 0.3 | 6.0 |
| AA35964 | 52.4 | 4.2 | AA35960 | 0.2 | 3.4 |
| AA35904 | 50.1 | 14.9 | | | |

Structural models of the top B13GT designs were generated and analyzed. To this end, a structure-based deep learning method trained to predict protein sequences for a given protein backbone was used to design further improved B13GT enzymes. 121 of these computational designs were chosen for experimental validation (SEQ ID NOs: 135-255). The machine learning model was retrained on crystal structures and structural models of proteins from thermo-philic organisms. An additional 69 B13GT enzymes were selected for experimental validation (SEQ ID NOs: 256-324). Polynucleotides encoding the B13GT enzymes (SEQ ID NOs: 135-324) with a C-terminal His tag were synthesized (Twist Bioscience). E. coli strains expressing the designed B13GTs were created as described in Example 1. The designed B13GT enzymes were expressed and purified as described in Example 1.

Each B13GT variant was assayed in a one-pot reaction with a B12GT and SuSy enzyme. B12GT and SuSy were used in excess such that in the absence of the B13GT variant, the starting RA50 feed was fully converted to RebD/E. The purified B13GT, B12GT and SuSy were reacted with 71.5 mg/ml RAA0, 450 mM Sucrose, and 0.5 mM ADP in 50 mM pH 6 phosphate buffer, and 50 mM NaCl for 24 hours at 60 °C. Steviol glycosides produced and consumed during the reaction were measured as described in Example 1. The designed enzymes solubly expressed and were able to efficiently produce Reb M (Table 5).

TABLE 5
Purified Protein Concentration and Reb M Conversion of Top B13GT
Design Variants Expressed in E. coli

| | | Puri- |
|---|---|---|
| | Reb M | fied |
| | Yield | Conc |
| ID | (%) | (uM) |
| AA40182 | 93.9 | 309.6 |
| AA40172 | 86.5 | 170.5 |
| AA40180 | 83.8 | 143.8 |
| AA40184 | 80.5 | 224.6 |
| AA40181 | 76.9 | 246.7 |
| AA40183 | 73.8 | 221.0 |
| AA40597 | 66.3 | 242.6 |
| AA40174 | 60.5 | 200.3 |
| AA40567 | 58.8 | 50.0 |
| AA40189 | 57.5 | 194.6 |
| AA40558 | 53.1 | 261.9 |
| AA40557 | 52.5 | 291.2 |
| AA40239 | 51.7 | 175.0 |
| AA40213 | 50.1 | 120.7 |
| AA40571 | 49.5 | 157.6 |
| AA40553 | 49.3 | 209.0 |
| AA40178 | 47.2 | 138.4 |
| AA40578 | 46.3 | 223.8 |
| AA40207 | 45.5 | 15.9 |
| AA40579 | 44.6 | 60.5 |
| AA40179 | 44.5 | 193.9 |
| AA40605 | 43.2 | 135.6 |
| AA40415 | 43.0 | 30.2 |
| AA40555 | 43.0 | 134.4 |
| AA40232 | 41.5 | 147.9 |
| AA40426 | 40.4 | 64.5 |
| AA40568 | 39.2 | 165.0 |
| AA40221 | 38.9 | 32.0 |
| AA40569 | 38.6 | 139.3 |
| AA40177 | 38.6 | 254.2 |
| AA40587 | 37.3 | 36.8 |
| AA40424 | 37.1 | 66.3 |
| AA40208 | 37.0 | 114.5 |
| AA40611 | 37.0 | 62.9 |
| AA40194 | 36.4 | 7.5 |
| AA40196 | 35.5 | 13.9 |
| AA40437 | 35.4 | 104.1 |
| AA40564 | 35.2 | 26.6 |
| AA40606 | 35.1 | 336.0 |
| AA40204 | 34.6 | 142.7 |
| AA40191 | 34.4 | 165.1 |
| AA40577 | 34.3 | 150.6 |
| AA40421 | 33.4 | 140.0 |
| AA40243 | 32.9 | 96.2 |
| AA40223 | 32.8 | 77.6 |
| AA40167 | 32.6 | 64.5 |
| AA40560 | 32.4 | 68.8 |
| AA40607 | 32.4 | 309.6 |
| AA40216 | 31.9 | 109.9 |
| AA40427 | 31.8 | 72.3 |
| AA40186 | 31.6 | 123.4 |
| AA40210 | 31.3 | 145.4 |
| AA40436 | 30.4 | 20.1 |
| AA40205 | 30.1 | 40.0 |
| AA40582 | 29.8 | 149.0 |
| AA40591 | 28.4 | 27.8 |
| AA40197 | 28.3 | 14.6 |
| AA40188 | 28.3 | 61.2 |
| AA40420 | 28.3 | 176.1 |
| AA40438 | 28.0 | 107.1 |
| AA40590 | 27.5 | 27.5 |
| AA40565 | 27.1 | 23.3 |
| AA40566 | 26.8 | 84.4 |
| AA40417 | 26.8 | 27.9 |
| AA40227 | 26.6 | 117.2 |
| AA40550 | 26.0 | 219.2 |
| AA40592 | 25.3 | 255.6 |
| AA40231 | 25.0 | 150.9 |
| AA40173 | 25.0 | 92.4 |
| AA40428 | 24.9 | 52.0 |
| AA40214 | 23.0 | 60.7 |
| AA40547 | 22.4 | 240.6 |
| AA40429 | 21.9 | 153.0 |
| AA40551 | 21.9 | 236.2 |
| AA40192 | 21.4 | 42.5 |
| AA40584 | 21.0 | 29.8 |
| AA40561 | 20.4 | 177.9 |
| AA40573 | 20.3 | 91.4 |
| AA40217 | 20.1 | 157.6 |
| AA40220 | 20.0 | 34.4 |
| AA40193 | 19.8 | 31.6 |
| AA40612 | 19.6 | 114.9 |
| AA40200 | 19.4 | 14.4 |
| AA40228 | 19.0 | 27.5 |
| AA40236 | 19.0 | 105.4 |
| AA40185 | 18.9 | 142.0 |
| AA40570 | 18.7 | 140.7 |
| AA40604 | 18.6 | 36.8 |
| AA40574 | 18.6 | 165.4 |
| AA40581 | 18.5 | 163.8 |
| AA40589 | 17.5 | 92.4 |
| AA40251 | 17.4 | 173.1 |
| AA40418 | 17.1 | 16.1 |
| AA40596 | 17.1 | 185.5 |
| AA40199 | 17.1 | 18.6 |
| AA40222 | 16.8 | 35.2 |
| AA40580 | 16.6 | 206.4 |
| AA40435 | 16.1 | 22.8 |
| AA40559 | 16.1 | 191.5 |
| AA40594 | 15.9 | 190.2 |
| AA40602 | 15.5 | 273.0 |
| AA40549 | 15.4 | 169.0 |
| AA40419 | 15.2 | 15.1 |
| AA40593 | 14.8 | 35.9 |
| AA40433 | 14.7 | 18.8 |
| AA40242 | 14.3 | 176.9 |
| AA40219 | 14.1 | 10.5 |
| AA40245 | 13.9 | 69.3 |
| AA40233 | 13.9 | 109.1 |
| AA40554 | 13.3 | 67.2 |
| AA40230 | 13.3 | 43.8 |
| AA40164 | 13.2 | 18.7 |
| AA40586 | 13.1 | 84.0 |
| AA40246 | 13.1 | 155.3 |
| AA40601 | 12.6 | 192.0 |
| AA40431 | 12.3 | 80.1 |
| AA40439 | 12.2 | 179.1 |
| AA40224 | 12.1 | 28.8 |
| AA40234 | 11.2 | 13.2 |
| AA40430 | 11.1 | 104.0 |
| AA40432 | 10.9 | 57.9 |
| AA40171 | 0.7 | 196.6 |
| AA40195 | 10.6 | 15.0 |
| AA40610 | 10.6 | 43.5 |
| AA40215 | 9.4 | 19.2 |
| AA40202 | 9.3 | 5.9 |
| AA40209 | 9.3 | 45.3 |
| AA40160 | 9.2 | 108.5 |
| AA40608 | 9.1 | 180.8 |
| AA40572 | 9.0 | 19.0 |
| AA40249 | 9.0 | 111.4 |
| AA40211 | 8.9 | 26.4 |
| AA40237 | 8.6 | 29.9 |
| AA40238 | 8.6 | 26.6 |
| AA40229 | 8.5 | 14.3 |
| AA40423 | 8.5 | 6.5 |
| AA40252 | 8.4 | 146.5 |
| AA40248 | 8.4 | 42.2 |
| AA40575 | 8.3 | 176.6 |
| AA40218 | 8.2 | 43.8 |
| AA40598 | 8.2 | 17.7 |
| AA40198 | 8.1 | 7.6 |
| AA40225 | 8.0 | 25.0 |
| AA40416 | 7.8 | 5.3 |
| AA40425 | 7.8 | 8.5 |
| AA40615 | 7.7 | 203.5 |
| AA40212 | 7.5 | 34.6 |
| AA40235 | 7.5 | 19.2 |
| AA40240 | 7.3 | 78.7 |
| AA40588 | 6.8 | 39.9 |
| AA40548 | 6.7 | 30.7 |
| AA40253 | 6.6 | 46.9 |
| AA40609 | 6.2 | 72.6 |
| AA40576 | 6.2 | 203.2 |
| AA40422 | 6.1 | 5.2 |
| AA40190 | 6.0 | 7.0 |
| AA40595 | 5.9 | 107.0 |
| AA40556 | 5.8 | 16.9 |
| AA40206 | 5.7 | 19.1 |
| AA40226 | 5.6 | 18.8 |
| AA40583 | 5.0 | 13.4 |
| AA40603 | 5.0 | 81.9 |
| AA40562 | 5.0 | 0.7 |
| AA40614 | 4.7 | 317.3 |
| AA40168 | 4.3 | 23.3 |
| AA40244 | 4.3 | 31.3 |
| AA40203 | 4.2 | 7.9 |
| AA40563 | 4.2 | 8.5 |
| AA40170 | 4.0 | 24.2 |
| AA40169 | 4.0 | 7.7 |
| AA40175 | 3.6 | 72.0 |
| AA40201 | 3.2 | 4.7 |
| AA40241 | 3.1 | 12.9 |
| AA40163 | 3.0 | 13.5 |
| AA40613 | 2.9 | 10.1 |
| AA40187 | 2.8 | 62.4 |
| AA40585 | 2.4 | 7.7 |
| AA40161 | 2.2 | 18.4 |
| AA40159 | 2.0 | 82.2 |
| AA40166 | 1.9 | 8.7 |
| AA40176 | 1.8 | 104.9 |
| AA40434 | 1.7 | 10.5 |
| AA40165 | 1.6 | 68.5 |
| AA40162 | 1.5 | 24.8 |
| AA40552 | 1.3 | 15.3 |
| AA40616 | 1.2 | 131.7 |
| AA40250 | 1.2 | 26.9 |
| AA40247 | 1.1 | 5.1 |
| AA40599 | 1.1 | 197.7 |
| AA40254 | 0.9 | 12.1 |

### Example 4: Computational Designs of Further Improved B13GTs

The protein-based deep learning model from Example 3 was fine-tuned with the results from Examples 1 and 3. This sequence-based model was used to design improved B13GT enzymes. One hundred of these computational designs were chosen for experimental validation (SEQ ID NOs: 325-424). Polynucleotides encoding the B13GT enzymes (SEQ II) NOs: 325-424) with a C-terminal His tag were synthesized (Twist Bioscience). E. coli strains expressing the designed B13GTs were created as described in Example 1. The designed B13GT enzymes were expressed and purified as described in Example 1.

Each B13GT variant was assayed in a one-pot reaction with a B2GT and SuSy enzyme. B12GT and SuSy were used in excess such that in the absence of the B13GT variant, the starting RA50 feed was fully converted to RebD/E. The purified B13GT, B12GT and SuSy were reacted with 71.5 mg/ml RA60, 450 mM Sucrose, and 0.5 mM ADP in 50 mM pH 6 phosphate buffer, and 250 mM NaAcetate for 24 hours at 60 °C. Steviol glycosides produced and consumed during the reaction were measured as described in Example 1. The designed enzymes solubly expressed and were able to efficiently produce Reb M (Table 6).

TABLE 6
Purified Protein Concentration and Reb M Conversion of Top B13GT
DesignVariants Expressed in E. coli

| | Reb | Puri- |
|---|---|---|
| | M | fied |
| | Yield | Conc |
| ID | (%) | (uM) |
| AA41201 | 17.8 | 311.8 |
| AA41191 | 16.9 | 321.1 |
| AA41199 | 15.7 | 288.3 |
| AA41195 | 15.1 | 317.7 |
| AA41202 | 14.7 | 319.9 |
| AA41198 | 13.9 | 290 |
| AA41200 | 13.8 | 296 |
| AA41185 | 13.7 | 315.6 |
| AA41193 | 13.3 | 262.9 |
| AA41183 | 11.9 | 296.4 |
| AA41194 | 11.9 | 297.9 |
| AA41190 | 11.6 | 169.6 |
| AA41192 | 11.4 | 245.5 |
| AA41196 | 0.7 | 291.6 |
| AA41257 | 0.7 | 150 |
| AA41186 | 10.4 | 251.1 |
| AA41187 | 9.9 | 248.9 |
| AA41197 | 9.5 | 287.2 |
| AA41184 | 9.4 | 75 |
| AA41189 | 8.7 | 276.7 |
| AA41188 | 6.9 | 279 |
| AA41274 | 6.6 | 272.5 |
| AA41271 | 6.3 | 240.2 |
| AA41279 | 5.8 | 66.4 |
| AA41273 | 5.6 | 88.7 |
| AA41239 | 5.6 | 195.4 |
| AA41256 | 5.3 | 214.5 |
| AA41264 | 5.3 | 178.9 |
| AA41222 | 5.3 | 233.2 |
| AA41260 | 5.2 | 212.6 |
| AA41267 | 5.2 | 125.7 |
| AA41218 | 5.2 | 117.5 |
| AA41261 | 5.1 | 31.6 |
| AA41229 | 5 | 40.9 |
| AA41233 | 4.9 | 283.6 |
| AA41234 | 4.9 | 55.4 |
| AA41241 | 4.7 | 255.8 |
| AA41214 | 4.6 | 242.2 |
| AA41265 | 4.6 | 38 |
| AA41244 | 4.4 | 246.7 |
| AA41268 | 4.4 | 28.8 |
| AA41208 | 4.3 | 47.8 |
| AA41275 | 4.3 | 137.7 |
| AA41258 | 4.3 | 307.4 |
| AA41217 | 4.3 | 239.4 |
| AA41277 | 4.3 | 242.5 |
| AA41280 | 4.2 | 98.6 |
| AA41209 | 4.2 | 0.9 |
| AA41242 | 4.1 | 17.3 |
| AA41282 | 4.1 | 250.5 |
| AA41251 | 4 | 205.7 |
| AA41235 | 4 | 105.4 |
| AA41227 | 4 | 85.3 |
| AA41236 | 4 | 316.4 |
| AA41266 | 3.9 | 12.8 |
| AA41225 | 3.9 | 291.8 |
| AA41249 | 3.8 | 315.1 |
| AA41213 | 3.8 | 39.7 |
| AA41263 | 3.8 | 247.8 |
| AA41216 | 3.8 | 153.9 |
| AA41246 | 3.8 | 31.7 |
| AA41226 | 3.7 | 87.7 |
| AA41221 | 3.7 | 210.2 |
| AA41272 | 3.6 | 205.2 |
| AA41281 | 3.5 | 150.4 |
| AA41278 | 3.4 | 38.4 |
| AA41224 | 3.4 | 83 |
| AA41231 | 3.3 | 36.5 |
| AA41240 | 3.3 | 194.8 |
| AA41269 | 3.3 | 239.4 |
| AA41245 | 3.2 | 202.7 |
| AA41232 | 3.2 | 93.9 |
| AA41259 | 3.2 | 287.9 |
| AA41237 | 3.1 | 241.5 |
| AA41204 | 3.1 | 196.5 |
| AA41223 | 3.1 | 20.2 |
| AA41203 | 3 | 53.9 |
| AA41253 | 3 | 113.1 |
| AA41254 | 2.9 | 230 |
| AA41212 | 2.9 | 255.8 |
| AA41228 | 2.9 | 95.8 |
| AA41205 | 2.8 | 77.6 |
| AA41247 | 2.7 | 74 |
| AA41219 | 2.7 | 252 |
| AA41276 | 2.6 | 71.7 |
| AA41220 | 2.6 | 222.2 |
| AA41210 | 2.5 | 112.9 |
| AA41211 | 2.5 | 68.2 |
| AA41238 | 2.4 | 8.6 |
| AA41215 | 2.4 | 157.3 |
| AA41248 | 2.2 | 35.1 |
| AA41207 | 2.1 | 202.7 |
| AA41252 | 2.1 | 58.8 |
| AA41255 | 2.1 | 80.7 |
| AA41270 | 2 | 31.8 |
| AA41230 | 1.9 | 171.4 |
| AA41206 | 1.9 | 99 |
| AA41250 | 1.8 | 58.7 |
| AA41243 | 1.7 | 26.6 |
| AA41262 | 1.7 | 133.3 |

Structural models of the top B13GT designs from Example 3 were generated and analyzed. The structure-based deep learning method trained to predict protein sequences for a given protein backbone was used to generate further improved B13GT enzymes. The method was used to design improved B13GT enzymes. 93 of these computational designs were chosen for experimental validation (SEQ ID NOs: 425-517). Polynucleotides encoding the B13GT enzymes with a C-terminal His tag were synthesized (Twist Bioscience). E. coli strains ex-pressing the designed B13GTs were created as described in Example 1. The designed B13GT enzymes were expressed and purified as described in Example 1.

Each B13GT variant was assayed in a one-pot reaction with a B12GT and SuSy enzyme. B12GT and SuSy were used in excess such that in the absence of the B13GT variant, the starting RA50 feed was fully converted to RebD/E. The purified B13GT, B12GT and SuSy were reacted with 71.5 mg/ml RA60, 450 mM Sucrose, and 0.5 mM ADP in 50 mM pH 6 phosphate buffer, and 250 mM NaAcetate for 24 hours at 60 °C. Steviol glycosides produced and consumed during the reaction were measured as described in Example 1. The designed enzymes solubly expressed and were able to efficiently produce Reb M (Table 7).

TABLE 7
Purified Protein Concentration and Reb M Conversion of Top B13GT
Design Variants Expressed in E. coli

| | | Puri- |
|---|---|---|
| | Reb M | fied |
| | Yield | Conc |
| ID | (%) | (uM) |
| AA41333 | 10.1 | 164.5 |
| AA41342 | 9.6 | 202.2 |
| AA41345 | 9.5 | 124.3 |
| AA41346 | 9.2 | 217.7 |
| AA41343 | 9.1 | 243.6 |
| AA41304 | 8.6 | 274.7 |
| AA41355 | 8.6 | 280.7 |
| AA41336 | 8.4 | 142.4 |
| AA41370 | 8.1 | 257 |
| AA41349 | 8 | 99.9 |
| AA41292 | 8 | 295.3 |
| AA41356 | 7.4 | 64.4 |
| AA41324 | 7.4 | 65.3 |
| AA41330 | 7.4 | 114.8 |
| AA41319 | 7 | 130.5 |
| AA41340 | 6.9 | 61.8 |
| AA41344 | 6.9 | 160.7 |
| AA41365 | 6.7 | 37.2 |
| AA41352 | 6.7 | 237.8 |
| AA41362 | 6.5 | 53.4 |
| AA41348 | 6.3 | 70.8 |
| AA41315 | 6.2 | 31.8 |
| AA41334 | 6.2 | 144 |
| AA41313 | 6.1 | 22.1 |
| AA41332 | 6 | 131.4 |
| AA41339 | 6 | 249.8 |
| AA41320 | 5.9 | 68.6 |
| AA41325 | 5.7 | 104.2 |
| AA41357 | 5.5 | 227.3 |
| AA41335 | 5.3 | 0.3 |
| AA41311 | 5.3 | 10.8 |
| AA41306 | 5.2 | 14 |
| AA41366 | 5.2 | 64.1 |
| AA41337 | 5.2 | 135.6 |
| AA41294 | 5.2 | 248.1 |
| AA41350 | 5.2 | 185.6 |
| AA41353 | 5 | 23.3 |
| AA41327 | 4.9 | 58.5 |
| AA41347 | 4.9 | 24.3 |
| AA41305 | 4.9 | 248.8 |
| AA41354 | 4.9 | 35.6 |
| AA41328 | 4.9 | 79.5 |
| AA41361 | 4.8 | 53.2 |
| AA41331 | 4.8 | 70 |
| AA41318 | 4.7 | 54.6 |
| AA41373 | 4.7 | 202.1 |
| AA41293 | 4.7 | 199.5 |
| AA41317 | 4.7 | 64 |
| AA41310 | 4.6 | 113.8 |
| AA41358 | 4.6 | 118.8 |
| AA41303 | 4.6 | 208.1 |
| AA41322 | 4.5 | 12.9 |
| AA41321 | 4.4 | 145 |
| AA41351 | 4.4 | 142.9 |
| AA41300 | 4.2 | 247.9 |
| AA41323 | 4.1 | 114 |
| AA41363 | 4.1 | 137.5 |
| AA41297 | 4 | 255.1 |
| AA41296 | 4 | 38.6 |
| AA41326 | 3.9 | 10.9 |
| AA41329 | 3.9 | 28 |
| AA41369 | 3.9 | 86.9 |
| AA41338 | 3.8 | 93.9 |
| AA41371 | 3.8 | 153.1 |
| AA41309 | 3.8 | 221.7 |
| AA41285 | 3.7 | 226.9 |
| AA41301 | 3.7 | 53.3 |
| AA41341 | 3.6 | 13.3 |
| AA41364 | 3.6 | 129.9 |
| AA41307 | 3.5 | 244 |
| AA41375 | 3.5 | 136.7 |
| AA41283 | 3.4 | 197.8 |
| AA41299 | 3.3 | 182.2 |
| AA41308 | 3.2 | 76.7 |
| AA41302 | 3.1 | 41.8 |
| AA41372 | 3 | 212.8 |
| AA41367 | 2.9 | 236.8 |
| AA41312 | 2.9 | 4.7 |
| AA41316 | 2.9 | 274.2 |
| AA41298 | 2.7 | 64.8 |
| AA41295 | 2.5 | 145.4 |
| AA41378 | 2.4 | 41.8 |
| AA41381 | 2.2 | 76.4 |
| AA41368 | 2.1 | 64.8 |
| AA41374 | 1.8 | 173.9 |
| AA41382 | 1.8 | 163.4 |
| AA41379 | 1.8 | 227.9 |
| AA41376 | 1.7 | 78.2 |
| AA41360 | 1.7 | 53.1 |
| AA41380 | 1.6 | 94.6 |
| AA41359 | 1.4 | 102.5 |
| AA41377 | 1.4 | 85.8 |
| AA41314 | 1.4 | 83.8 |

### Example 5: Representing Successful B13GTs Designs with a PSSM

The successful B13GT designs from Example 1 were used to generate a PSSM (Table 8). The PSSM is a concise way to represent the successful designs and related sequences. Sequences that have a PSSM score greater than 332 are considered related to the active B13GT computational designs described herein. To score a sequence with the PSSM, it must first be aligned with the representative sequence, SEQ ID NO: 2. For example, the following successful designs, AA29162, AA29163, AA29161, AA29165, have the following PSSM scores: 332.2, 335.5, 332.5, 337.3, while the wild-type B13GT SEQ ID NO: 1, has a PSSM score of only 326.7.

The successful B13GT designs from Example 3 were used to generate a PSSM (Table 9). The PSSM is a concise way to represent successful designs and related sequences. Sequences that have a PSSM score greater than 54.5 are considered related to the active B13GT computational designs described in herein. To score a sequence with the PSSM, it must first be aligned with the representative sequence, SEQ ID NO: 2. For example, the following successful designs, AA40232, AA40239, AA40205, AA40243, have the following PSSM scores: 70.74, 80.53, 78.75, 76.08, while the wild-type B3GT SEQ ID NO: 1, has a PSSM score of only 51.16.

The successful B13GT designs from Example 4 were used to generate a PSSM (Table 10). The PSSM is a concise way to represent successful designs and related sequences. Sequences that have a PSSM score greater than 58.3 are considered related to the active B13GT computational designs described herein. To score a sequence with the PSSM, it must first be aligned with the representative sequence, SEQ ID NO: 2. For example, the following successful designs, AA41333, AA41342, AA41345, AA41346, have the following PSSM scores: 90.04, 92.68, 89.16, 97.08, while the wild-type B13GT SEQ ID NO: 1, has a PSSM score of only 51.16.

TABLE 8
Position Specific Scoring Matrix (PSSM) of Successful B13GT Designs

| Seq | | | | | | | | | | | |
|---|---|---|---|---|---|---|---|---|---|---|---|
| Pos | A | R | N | D | C | Q | E | G | H | I | L |
| 1 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 |
| 3 | 0 | 0 | 0.7 | 0 | 0 | 0 | 0 | 0 | 0 | 0 | 0 |
| 4 | 0 | 0 | 0 | 0 | 0 | 0 | 0 | 0 | 0 | 0 | 0 |
| 5 | 0 | 0 | 0.7 | 0 | 0 | 0 | 0 | 0 | 0 | 0 | 0 |
| 6 | 0 | 0 | 0 | 0 | 0 | 0 | 0 | 0 | 0 | 0 | 0 |
| 7 | 0 | 0 | 0 | 0 | 0 | 0 | 0 | 0 | 0 | 0 | 0 |
| 8 | 0 | 0 | 0 | 0 | 0 | 0 | 0 | 0 | 0 | 0 | 0 |
| 9 | 0 | 1 | 0 | 0 | 0 | 0 | 0 | 0 | 0 | 0 | 0 |
| 10 | 0 | 0.7 | 0 | 0 | 0 | 0 | 0 | 0 | 0 | 0 | 0 |
| 11 | 0 | 0.7 | 0 | 0 | 0 | 0 | 0 | 0 | 0 | 0 | 0 |
| 12 | 0 | 0.7 | 0 | 0 | 0 | 0 | 0 | 0 | 0 | 0 | 0 |
| 13 | 0 | 0.7 | 0 | 0 | 0 | 0 | 0 | 0 | 0 | 0 | 0 |
| 14 | 0 | 0 | 0 | 0 | 0 | 0 | 0 | 0 | 0 | 1 | 0 |
| 15 | 0 | 0 | 0 | 0 | 0 | 0 | 0 | 0 | 0 | 0.7 | 0 |
| 16 | 0 | 0 | 0 | 0 | 0 | 0 | 0 | 0 | 0 | 0 | 0.7 |
| 17 | 0 | 0 | 0 | 0 | 0 | 0 | 0 | 0 | 0 | 0 | 0 |
| 18 | 0 | 0 | 0 | 0 | 0 | 0 | 0 | 0 | 0 | 0 | 0 |
| 19 | 0 | 0 | 0 | 0 | 0 | 0 | 0 | 0 | 0 | 0 | 0 |
| 20 | 0 | 0 | 0 | 0 | 0 | 0 | 0 | 0 | 0 | 0 | 0 |
| 21 | 0 | 0 | 0 | 0 | 0 | 0 | 0 | 0 | 0 | 0 | 0 |
| 22 | 0 | 0 | 0 | 0 | 0 | 0 | 0 | 0 | 0 | 0 | 0 |
| 23 | 0 | 0 | 0 | 0 | 0 | 0 | 0 | 0.7 | 0 | 0 | 0 |
| 24 | 0 | 0 | 0 | 0 | 0 | 0 | 0 | 0 | 0.7 | 0 | 0 |
| 25 | 0 | 0 | 0 | 0 | 0 | 0 | 0 | 0 | 0 | 1 | 0.7 |
| 26 | 0 | 0 | 0.7 | 0 | 0 | 0 | 0 | 0 | 0 | 0 | 0 |
| 27 | 0 | 0 | 0 | 0 | 0 | 0 | 0 | 0 | 0 | 0 | 0 |
| 28 | 0 | 0 | 0 | 0 | 0 | 0 | 0 | 0 | 0 | 1 | 0 |
| 29 | 0 | 0 | 0 | 0 | 0 | 0 | 0 | 0 | 0 | 0 | 0.7 |
| 30 | 0 | 0 | 0 | 0 | 0 | 0.7 | 0 | 0 | 0 | 0 | 0 |
| 31 | 0 | 0 | 0 | 0 | 0 | 0 | 0 | 0 | 0 | 0 | 0.7 |
| 32 | 0.7 | 0 | 0 | 0 | 0 | 0 | 0 | 0 | 0 | 0 | 0 |
| 33 | 0 | 0 | 0.7 | 0 | 0 | 0 | 0 | 0 | 0 | 0 | 0 |
| 34 | 0 | 0 | 0 | 0 | 0 | 0 | 0 | 0 | 0 | 0 | 1 |
| 35 | 0 | 0 | 0 | 0 | 0 | 0 | 0 | 0 | 0 | 0 | 0.7 |
| 36 | 0 | 0 | 0 | 0 | 0 | 0 | 0 | 0 | 0 | 0 | 0 |
| 37 | 0 | 0.7 | 0 | 0 | 0 | 0 | 0 | 0 | 0 | 0 | 0 |
| 38 | 0 | 0.7 | 0 | 0 | 0 | 0 | 0 | 0 | 0 | 0 | 0 |
| 39 | 0 | 0 | 0 | 0 | 0 | 0 | 0 | 0.7 | 0 | 0 | 0 |
| 40 | 0 | 0 | 0 | 0 | 0 | 0 | 0 | 0 | 0 | 0 | 0 |
| 41 | 0.7 | 0 | 0 | 0 | 0 | 0 | 0.7 | 0 | 0 | 0 | 0 |
| 42 | 0 | 0 | 0 | 0 | 0 | 0 | 0 | 0 | 0 | 0.7 | 0 |
| 43 | 0 | 0 | 0 | 0 | 0 | 0 | 0 | 0 | 0 | 0 | 0 |
| 44 | 0 | 0 | 0 | 0 | 0 | 0 | 0 | 0 | 0 | 0.7 | 0 |
| 45 | 0 | 0 | 0 | 0 | 0 | 0 | 0 | 0 | 0 | 0 | 0.7 |
| 46 | 0 | 0 | 0 | 0 | 0 | 0 | 0 | 0 | 0.7 | 0 | 0 |
| 47 | 0 | 0 | 0 | 0 | 0 | 0 | 0 | 0 | 0 | 0 | 0 |
| 48 | 0 | 0 | 0.7 | 0 | 0 | 0 | 0 | 0 | 0 | 0 | 0 |
| 49 | 0 | 0 | 0 | 0 | 0 | 0 | 0 | 0 | 0 | 0 | 0 |
| 50 | 0 | 0 | 0.7 | 0 | 0 | 0 | 0 | 0 | 0 | 0 | 0 |
| 51 | 0.7 | 0 | 0 | 0 | 0 | 0 | 0 | 0 | 0 | 0 | 0 |
| 52 | 0 | 0 | 0 | 0 | 0 | 0 | 0 | 0 | 0 | 0 | 0 |
| 53 | 0 | 0 | 0 | 0 | 0 | 0 | 0 | 0 | 0 | 0 | 0 |
| 54 | 0 | 0 | 0 | 0 | 0 | 0 | 0 | 0 | 0 | 0 | 0 |
| 55 | 0 | 0 | 0 | 0 | 0 | 0 | 0 | 0 | 0 | 0 | 0 |
| 56 | 0 | 0 | 1 | 0 | 0 | 0 | 0 | 0 | 0 | 0 | 0.7 |
| 57 | 0 | 0 | 0 | 0 | 0 | 0 | 0 | 0 | 0 | 0 | 0 |
| 58 | 0 | 0 | 0 | 0 | 0 | 0 | 0 | 0 | 0 | 0 | 0 |
| 59 | 0 | 0 | 0 | 0 | 0 | 0 | 0 | 0 | 0.7 | 0 | 0 |
| 60 | 0 | 0 | 0 | 0 | 0 | 0 | 0 | 0 | 0 | 0 | 0 |
| 61 | 0 | 0 | 0 | 0 | 0 | 0.7 | 0 | 0 | 0 | 0 | 0 |
| 62 | 0 | 0 | 0 | 0 | 0 | 0 | 0 | 0 | 0 | 0 | 0 |
| 63 | 0 | 0.7 | 0 | 0 | 0 | 0 | 0 | 0 | 0 | 0 | 0 |
| 64 | 0 | 0 | 0 | 0 | 0 | 0 | 0 | 0 | 0 | 0 | 0 |
| 65 | 0 | 0 | 0 | 0 | 0 | 0 | 0 | 0 | 0 | 0.7 | 0 |
| 66 | 0 | 0 | 0 | 0 | 0 | 0 | 0 | 0 | 0 | 0 | 0.7 |
| 67 | 0 | 0 | 0 | 0.7 | 0 | 0 | 0 | 0 | 0 | 0 | 0 |
| 68 | 0 | 0 | 0.7 | 0 | 0 | 0 | 0 | 0 | 0 | 0 | 0 |
| 69 | 0 | 0 | 0 | 0.7 | 0 | 0 | 0 | 0 | 0 | 0 | 0 |
| 70 | 0 | 0 | 0 | 0 | 0 | 0 | 0 | 0 | 0 | 0 | 0 |
| 71 | 0 | 0 | 0 | 0 | 0 | 0.7 | 0 | 0 | 0 | 0 | 0 |
| 72 | 0 | 0 | 0 | 1 | 0 | 0 | 0 | 0 | 0 | 0 | 0 |
| 73 | 0 | 0 | 0 | 0 | 0 | 0 | 0.7 | 0 | 0 | 0 | 0 |
| 74 | 0 | 0 | 0 | 0 | 0 | 0 | 0 | 0 | 0 | 0 | 0 |
| 75 | 0 | 0 | 0 | 0 | 0 | 0 | 0 | 0 | 0 | 0 | 0.7 |
| 76 | 0 | 0.7 | 0 | 0 | 0 | 0 | 0 | 0 | 0 | 0 | 0 |
| 77 | 0 | 0 | 0.7 | 0 | 0 | 0 | 0 | 0 | 0 | 0 | 0 |
| 78 | 0 | 0 | 0 | 0 | 0 | 0 | 0 | 0 | 0 | 0 | 0.7 |
| 79 | 0 | 0 | 0 | 0 | 0 | 0 | 0 | 0 | 0 | 0 | 0 |
| 80 | 0 | 0 | 0 | 0 | 0 | 0 | 0 | 0 | 0 | 0 | 0 |
| 81 | 0 | 0 | 0 | 0 | 0 | 0 | 0 | 0 | 0.7 | 0 | 0 |
| 82 | 0 | 0 | 0 | 0 | 0 | 0 | 0 | 0.7 | 0 | 0 | 0 |
| 83 | 0 | 0 | 0 | 0 | 0 | 0 | 0 | 0 | 0 | 0 | 0 |
| 84 | 0 | 0 | 0 | 0 | 0 | 0 | 0 | 0.7 | 0 | 0 | 0 |
| 85 | 0.7 | 0 | 0 | 0 | 0 | 0 | 0 | 0 | 0 | 0 | 0 |
| 86 | 0 | 0 | 0 | 0 | 0 | 0 | 0 | 0.7 | 0 | 0 | 0 |
| 87 | 0.7 | 0 | 0 | 0 | 0 | 0 | 0 | 0 | 0 | 0 | 0 |
| 88 | 0 | 0.7 | 0 | 0 | 0 | 0 | 0 | 0 | 0 | 0 | 0 |
| 89 | 0 | 0 | 0 | 0 | 0 | 0 | 0 | 0 | 0 | 0.7 | 0 |
| 90 | 0 | 0 | 0 | 0 | 0 | 0 | 0 | 0 | 0 | 0 | 0 |
| 91 | 0 | 0 | 0 | 0 | 0 | 0.7 | 0 | 0 | 0 | 1 | 1 |
| 92 | 0 | 0 | 0 | 0 | 0 | 0 | 0 | 0 | 0 | 0.7 | 0 |
| 93 | 0 | 0 | 0.7 | 0 | 0 | 0 | 0 | 0 | 0 | 0 | 0 |
| 94 | 0 | 0 | 0 | 0 | 0 | 1 | 1 | 0 | 0 | 0 | 0 |
| 95 | 0 | 0 | 0 | 0 | 0 | 0 | 0 | 0 | 0.7 | 0 | 0 |
| 96 | 0 | 0 | 0 | 0 | 0 | 0 | 0 | 0.7 | 0 | 0 | 0 |
| 97 | 0.7 | 0 | 0 | 0 | 0 | 0 | 0 | 0 | 0 | 0 | 0 |
| 98 | 0 | 0 | 0 | 0.7 | 0 | 0 | 0 | 0 | 0 | 0 | 0 |
| 99 | 0 | 0 | 0 | 0 | 0 | 0 | 0.7 | 0 | 0 | 0 | 0 |
| 100 | 0 | 0 | 0 | 0 | 0 | 0 | 0 | 0 | 0 | 0 | 1 |
| 101 | 0 | 0.7 | 0 | 0 | 0 | 0 | 0 | 0 | 0 | 0 | 0 |
| 102 | 0 | 1 | 0 | 1 | 0 | 1 | 0 | 0 | 0 | 0 | 0 |
| 103 | 0 | 0 | 0 | 0 | 0 | 0 | 0.7 | 0 | 0 | 0 | 0 |
| 104 | 0 | 0 | 0 | 0 | 0 | 0 | 0 | 0 | 0 | 0 | 0.7 |
| 105 | 0 | 0 | 0 | 0 | 0 | 0 | 0.7 | 0 | 0 | 0 | 0 |
| 106 | 0 | 0 | 0 | 0 | 0 | 1 | 1 | 0 | 0 | 0.7 | 1 |
| 107 | 0.7 | 0 | 0 | 0 | 0.7 | 0 | 0 | 0 | 0 | 0 | 1 |
| 108 | 0 | 0 | 0 | 0 | 0 | 0 | 0 | 0 | 0 | 0 | 1 |
| 109 | 1 | 0.7 | 0 | 0 | 0 | 0 | 0 | 0 | 0 | 0 | 1 |
| 110 | 1 | 0 | 0 | 0.7 | 0 | 0 | 1 | 0 | 0 | 0 | 0 |
| 111 | 0 | 0 | 0 | 0 | 0 | 0 | 0 | 0 | 0 | 0 | 0 |
| 112 | 0 | 0 | 0 | 0 | 0 | 0 | 1 | 0 | 0 | 0 | 0 |
| 113 | 0 | 0 | 0 | 1 | 0 | 0 | 1 | 0 | 0 | 0 | 0 |
| 114 | 0 | 0 | 0 | 0.7 | 0 | 0 | 0 | 0 | 0 | 0 | 0 |
| 115 | 0 | 0 | 0 | 0 | 0 | 0 | 0.7 | 0 | 0 | 0 | 0 |
| 116 | 0 | 0 | 0 | 0 | 0 | 0 | 0.7 | 0 | 0 | 0 | 0 |
| 117 | 0 | 0 | 0 | 0 | 0 | 0 | 0 | 0 | 0 | 0 | 0 |
| 118 | 0.7 | 0 | 0 | 0 | 0 | 0 | 0 | 0 | 0 | 0 | 0 |
| 119 | 0 | 0 | 0 | 0 | 0.7 | 0 | 0 | 0 | 0 | 0 | 0 |
| 120 | 0 | 0 | 0 | 0 | 0 | 0 | 0 | 0 | 0 | 0 | 1 |
| 121 | 0 | 0 | 0 | 0 | 0 | 0 | 0 | 0 | 0 | 0.7 | 0 |
| 122 | 0 | 0 | 0 | 0 | 0 | 0 | 0 | 0 | 0 | 0 | 0 |
| 123 | 0 | 0 | 0 | 0.7 | 0 | 0 | 0 | 0 | 0 | 0 | 0 |
| 124 | 0.7 | 0 | 0 | 0 | 0 | 0 | 0 | 0 | 0 | 0 | 0 |
| 125 | 0 | 0 | 0 | 0 | 0 | 0 | 0 | 0 | 0 | 0 | 0.7 |
| 126 | 0 | 0 | 0 | 0 | 0 | 0 | 0 | 0 | 0 | 0 | 0 |
| 127 | 0 | 0 | 0 | 0 | 0 | 0 | 0 | 0 | 0 | 0 | 0 |
| 128 | 0 | 0 | 0 | 0 | 0 | 0 | 0 | 0 | 0 | 0 | 0 |
| 129 | 0.7 | 0 | 0 | 0 | 0 | 0 | 0 | 0 | 0 | 0 | 0 |
| 130 | 0 | 0 | 0 | 0 | 0 | 0.7 | 0 | 0 | 0 | 0 | 0 |
| 131 | 0 | 0 | 0 | 0 | 0 | 0 | 0 | 0 | 0 | 0 | 0 |
| 132 | 0 | 0 | 0 | 0 | 0 | 0 | 0 | 0 | 0 | 0 | 0 |
| 133 | 0.7 | 0 | 0 | 0 | 0 | 0 | 0 | 0 | 0 | 0 | 0 |
| 134 | 0 | 0 | 0 | 0.7 | 0 | 0 | 0 | 0 | 0 | 0 | 0 |
| 135 | 0 | 0 | 0 | 0 | 0 | 0 | 0 | 0 | 0 | 0 | 0 |
| 136 | 0 | 0 | 0 | 0 | 0 | 0 | 0 | 0 | 0 | 0 | 0.7 |
| 137 | 0 | 0 | 0.7 | 0 | 0 | 0 | 0 | 0 | 0 | 0 | 0 |
| 138 | 0 | 0 | 0 | 0 | 0 | 0 | 0 | 0 | 0 | 0 | 0.7 |
| 139 | 0 | 1 | 0 | 0 | 0 | 0 | 0 | 0 | 0 | 0 | 0 |
| 140 | 0 | 0.7 | 0 | 0 | 0 | 0 | 0 | 0 | 0 | 0 | 0 |
| 141 | 0 | 0 | 0 | 0 | 0 | 0 | 0 | 0 | 0 | 0 | 0.7 |
| 142 | 0 | 0 | 0 | 0 | 0 | 0 | 0 | 0 | 0 | 0 | 0 |
| 143 | 0 | 0 | 0 | 0 | 0 | 0 | 0 | 0 | 0 | 0 | 0.7 |
| 144 | 0 | 0 | 0 | 0 | 0 | 0.7 | 0 | 0 | 0 | 0 | 0 |
| 145 | 0 | 0 | 0 | 0 | 0 | 0 | 0 | 0 | 0 | 0 | 0 |
| 146 | 0 | 0 | 0 | 0 | 0 | 0 | 0 | 0.7 | 0 | 0 | 0 |
| 147 | 0 | 0 | 0 | 0 | 0 | 0 | 0 | 0 | 0 | 0 | 0 |
| 148 | 0 | 0 | 0 | 0 | 0 | 0 | 0 | 0 | 0 | 0 | 0.7 |
| 149 | 0 | 0 | 0 | 0 | 0 | 0 | 0 | 0 | 0 | 0 | 0 |
| 150 | 0 | 0 | 0.7 | 0 | 0 | 0 | 0 | 0 | 0 | 0 | 0 |
| 151 | 0 | 0 | 0 | 0 | 0 | 0 | 0 | 0 | 0 | 0 | 0 |
| 152 | 0 | 0 | 0 | 0 | 0 | 0 | 0 | 0 | 0.7 | 0 | 1 |
| 153 | 0.7 | 0 | 0 | 0 | 0.7 | 0 | 0 | 0 | 0 | 0 | 0 |
| 154 | 0 | 0 | 0 | 0 | 0 | 0 | 0 | 0 | 0 | 0 | 0.7 |
| 155 | 1 | 0 | 0 | 0 | 0 | 0 | 0 | 0 | 0 | 0 | 0 |
| 156 | 0 | 0 | 0 | 0 | 0.7 | 0 | 0 | 0 | 0 | 0 | 0 |
| 157 | 0 | 0 | 0 | 0 | 0 | 0 | 0 | 0 | 0 | 0 | 0.7 |
| 158 | 0 | 0 | 0 | 0 | 0 | 0 | 0 | 0 | 0 | 0 | 0 |
| 159 | 0 | 0 | 0 | 0 | 0 | 1 | 0 | 0 | 0 | 0 | 1 |
| 160 | 0 | 0 | 0 | 0 | 0 | 0 | 0 | 0 | 0 | 0 | 0 |
| 161 | 0 | 0 | 0 | 1 | 0 | 0 | 0 | 0 | 0 | 0 | 0.7 |
| 162 | 0 | 0 | 0 | 1 | 0 | 0 | 0.7 | 0 | 0 | 0 | 0 |
| 163 | 0 | 0 | 0 | 0 | 0 | 0 | 0 | 0 | 0 | 0 | 0.7 |
| 164 | 0 | 0 | 0 | 0 | 0 | 0 | 0 | 0.7 | 0 | 0 | 0 |
| 165 | 0 | 0 | 0 | 0 | 0 | 0 | 0 | 0 | 0 | 0 | 0 |
| 166 | 0 | 0 | 0 | 0 | 0 | 0 | 0 | 0 | 0 | 0 | 0.7 |
| 167 | 0 | 0 | 0 | 0.7 | 0 | 0 | 0 | 0 | 0 | 0 | 0 |
| 168 | 0 | 0 | 0 | 0 | 0 | 0 | 0 | 0 | 0 | 0 | 1 |
| 169 | 0 | 0 | 0 | 1 | 0 | 0 | 0.7 | 0 | 0 | 0 | 0 |
| 170 | 0 | 0 | 0 | 0.7 | 0 | 0 | 0 | 0 | 0 | 0 | 0 |
| 171 | 0 | 0 | 0 | 0 | 0 | 0 | 0 | 0 | 0 | 0 | 0 |
| 172 | 0 | 0 | 0 | 0 | 0 | 0 | 0 | 0 | 0.7 | 0 | 0 |
| 173 | 0 | 0.7 | 0 | 0 | 0 | 0 | 0 | 0 | 0 | 0 | 0 |
| 174 | 0 | 0 | 0 | 0 | 0 | 0 | 0 | 0 | 0 | 0 | 1 |
| 175 | 0 | 0 | 0 | 0.7 | 0 | 0 | 0.7 | 0 | 0 | 0 | 0 |
| 176 | 0 | 0 | 0 | 0 | 0 | 0 | 0.7 | 0 | 0 | 0 | 0 |
| 177 | 0 | 0 | 0 | 0 | 0 | 1 | 0 | 0 | 0 | 0 | 0 |
| 178 | 1 | 0 | 0 | 0 | 0 | 0 | 0 | 0 | 0 | 0 | 0 |
| 179 | 0 | 0 | 0 | 0 | 0 | 0 | 0 | 0 | 0 | 0 | 0 |
| 180 | 0 | 0 | 0 | 0 | 0 | 0 | 0 | 0.7 | 0 | 0 | 0 |
| 181 | 0 | 0 | 0 | 0 | 0 | 0 | 0 | 0 | 0 | 0 | 0 |
| 182 | 0 | 0 | 0 | 0 | 0 | 0 | 0 | 0 | 0 | 0 | 0 |
| 183 | 0 | 0 | 0 | 0 | 0 | 0 | 0 | 0 | 0 | 0 | 0 |
| 184 | 0 | 0 | 0 | 0 | 0 | 0 | 0 | 0 | 0 | 0 | 0.7 |
| 185 | 0 | 0 | 0 | 0 | 0 | 0 | 0 | 0 | 0 | 0 | 0 |
| 186 | 0 | 0 | 0 | 0 | 0 | 0 | 0 | 0 | 0 | 0 | 0 |
| 187 | 0 | 0 | 0 | 0 | 0 | 0 | 0 | 0 | 0 | 0 | 0 |
| 188 | 0 | 0 | 0 | 0.7 | 0 | 0 | 0 | 0 | 0 | 0 | 0 |
| 189 | 0 | 0 | 0 | 0 | 0 | 0 | 0 | 0 | 0 | 0.7 | 0 |
| 190 | 0 | 0.7 | 0 | 0 | 0 | 0 | 0 | 0 | 0 | 0 | 1 |
| 191 | 0 | 0.7 | 0 | 0 | 0 | 0 | 0 | 0 | 0 | 0 | 0 |
| 192 | 0.7 | 0 | 0 | 0 | 0 | 0 | 0 | 0 | 0 | 0 | 0 |
| 193 | 0 | 0 | 0 | 0 | 0 | 0 | 0 | 0 | 0 | 0 | 0 |
| 194 | 0 | 0 | 0 | 0 | 0 | 0 | 0 | 0 | 0 | 0 | 0 |
| 195 | 0 | 0 | 0 | 0 | 0 | 0 | 0 | 0 | 0.7 | 0 | 0 |
| 196 | 0 | 0 | 0 | 1 | 0 | 0 | 0 | 0 | 0 | 0.7 | 0 |
| 197 | 0 | 0 | 0 | 1 | 0 | 0.7 | 0 | 0 | 0 | 0 | 0 |
| 198 | 0 | 0 | 0 | 0 | 0 | 0 | 0.7 | 0 | 0 | 0 | 0 |
| 199 | 0.7 | 0 | 0 | 0 | 0 | 0 | 0 | 0 | 0 | 0 | 0 |
| 200 | 0 | 0 | 0 | 0 | 0 | 0 | 0 | 0 | 0 | 0 | 0 |
| 201 | 0 | 0 | 0 | 0 | 0 | 0 | 0 | 0 | 0 | 0 | 0 |
| 202 | 0 | 0 | 0 | 0 | 0 | 0 | 0 | 0 | 0 | 0.7 | 0 |
| 203 | 0 | 0 | 0 | 0 | 0 | 0 | 0 | 0 | 0 | 0 | 0.7 |
| 204 | 0 | 0 | 0 | 0.7 | 0 | 0 | 1 | 1 | 0 | 0 | 0 |
| 205 | 0 | 0 | 0 | 0 | 0 | 0 | 0 | 0 | 0 | 0 | 0 |
| 206 | 0 | 0 | 0 | 0 | 0 | 0 | 0 | 0 | 0 | 0 | 0 |
| 207 | 0 | 0 | 0 | 0 | 0 | 0 | 0 | 0 | 0 | 0.7 | 0 |
| 208 | 0 | 0 | 0 | 0 | 0 | 0 | 0.7 | 0 | 0 | 0 | 0 |
| 209 | 0 | 0 | 0 | 0 | 0 | 1 | 0.7 | 0 | 0 | 0 | 0 |
| 210 | 0 | 0 | 0 | 0 | 0 | 0 | 0 | 0 | 0 | 0 | 0 |
| 211 | 0 | 0.7 | 0 | 0 | 0 | 0 | 0 | 0 | 0 | 0 | 0 |
| 212 | 0.7 | 0 | 0 | 0 | 0 | 0 | 0 | 0 | 0 | 0 | 0 |
| 213 | 0 | 0 | 0 | 0 | 0 | 0 | 0 | 0 | 0 | 0 | 0 |
| 214 | 0 | 0 | 0 | 0 | 0 | 0 | 0 | 0 | 0 | 0 | 0 |
| 215 | 0 | 0 | 0 | 0 | 0 | 0 | 0 | 0.7 | 0 | 0 | 0 |
| 216 | 0 | 0 | 0 | 0 | 0 | 0 | 0 | 0 | 0 | 1 | 0 |
| 217 | 0 | 0 | 0 | 0 | 0 | 0 | 0 | 0 | 0 | 0.7 | 0 |
| 218 | 0 | 0 | 0 | 0 | 0 | 0 | 0 | 0 | 0 | 0 | 0 |
| 219 | 0 | 0 | 0.7 | 0 | 0 | 0 | 0 | 0 | 0 | 0 | 0 |
| 220 | 0 | 0 | 0 | 0 | 0 | 0 | 0 | 0 | 0 | 0 | 0 |
| 221 | 0.7 | 0 | 0 | 0 | 0 | 0 | 0 | 0 | 0 | 0 | 0 |
| 222 | 0 | 0 | 0 | 0 | 0 | 0 | 0 | 0 | 0 | 0 | 0 |
| 223 | 0 | 0 | 0 | 0 | 0 | 0 | 0.7 | 0 | 0 | 0 | 0 |
| 224 | 0 | 0 | 0 | 0 | 0 | 0 | 0 | 0 | 0 | 0 | 0.7 |
| 225 | 0 | 0 | 0 | 0 | 0 | 0 | 0.7 | 0 | 0 | 0 | 0 |
| 226 | 0 | 0 | 0 | 0 | 0 | 0 | 0.7 | 0 | 0 | 0 | 0 |
| 227 | 0 | 0 | 0 | 0 | 0 | 0 | 0 | 0 | 0 | 0 | 0 |
| 228 | 0 | 0 | 0 | 0 | 0 | 0 | 0.7 | 0 | 0 | 0 | 0 |
| 229 | 0 | 0 | 0 | 0 | 0 | 0 | 0 | 0 | 0 | 0 | 0.7 |
| 230 | 0 | 0 | 0 | 0 | 0 | 0 | 0.7 | 0 | 0 | 0 | 0 |
| 231 | 0 | 0 | 0 | 0 | 0 | 0 | 0 | 0 | 0 | 0 | 0 |
| 232 | 0 | 0 | 0 | 0 | 0 | 0 | 0 | 0 | 0 | 0.7 | 0 |
| 233 | 0 | 1 | 0 | 0 | 0 | 0.7 | 0 | 0 | 0 | 1 | 0 |
| 234 | 0 | 0.7 | 0 | 0 | 0 | 0 | 0 | 0 | 0 | 0 | 0 |
| 235 | 0 | 0 | 0 | 1 | 0 | 0 | 0.7 | 0 | 0 | 0 | 0 |
| 236 | 0 | 0 | 0 | 0 | 0 | 0 | 0 | 0 | 0 | 0.7 | 0 |
| 237 | 0 | 0 | 0 | 0 | 0 | 0 | 0 | 0 | 0 | 0 | 0 |
| 238 | 0.7 | 0 | 0 | 0 | 0 | 0 | 0 | 0 | 0 | 0 | 0 |
| 239 | 0 | 0 | 0 | 0 | 0 | 0 | 0 | 0 | 0 | 0 | 0 |
| 240 | 0 | 0 | 0 | 0 | 0 | 0 | 0 | 0 | 0 | 0 | 0 |
| 241 | 0 | 0 | 0 | 0 | 0 | 0 | 0 | 0 | 0 | 0 | 0 |
| 242 | 0 | 0 | 0 | 0 | 0 | 0 | 0 | 0 | 0 | 0 | 0.7 |
| 243 | 0 | 0 | 0 | 0 | 0 | 0 | 0 | 0 | 0 | 1 | 0.7 |
| 244 | 0 | 0 | 0 | 0 | 0 | 0 | 0 | 0 | 0 | 0 | 0 |
| 245 | 0 | 0 | 0 | 0 | 0 | 0 | 0 | 0 | 0 | 0 | 0.7 |
| 246 | 0 | 0 | 0 | 0 | 0 | 0 | 0 | 0 | 0 | 0 | 0 |
| 247 | 0 | 0 | 0 | 0 | 0 | 0 | 0 | 0 | 0 | 0 | 0 |
| 248 | 0 | 0 | 0 | 0 | 0 | 0 | 0 | 0 | 0.7 | 0 | 0 |
| 249 | 0 | 0 | 0 | 0 | 0 | 0 | 0 | 0 | 0 | 0 | 1 |
| 250 | 0 | 0.7 | 0 | 0 | 0 | 0 | 0 | 0 | 0 | 0 | 0 |
| 251 | 0.7 | 0 | 0 | 0 | 0 | 0 | 0 | 0 | 0 | 0 | 0 |
| 252 | 0 | 0 | 0 | 0 | 0 | 0 | 0 | 0 | 0 | 0 | 0 |
| 253 | 0 | 0 | 0 | 0 | 0 | 0 | 0 | 0 | 0 | 0 | 0 |
| 254 | 0 | 0 | 0 | 0 | 0 | 0 | 0 | 0 | 0 | 0 | 0 |
| 255 | 0 | 0 | 0 | 0 | 0 | 0 | 0 | 0 | 0 | 0 | 0 |
| 256 | 0 | 0 | 0 | 0 | 0 | 0 | 0 | 0 | 0 | 0 | 0.7 |
| 257 | 0 | 0 | 0 | 0 | 0 | 0 | 0 | 0 | 0 | 0 | 0.7 |
| 258 | 0 | 0 | 0 | 0.7 | 0 | 0 | 0 | 0 | 0 | 0 | 0 |
| 259 | 0 | 0 | 0 | 0 | 0 | 0 | 0.7 | 0 | 0 | 0 | 0 |
| 260 | 0 | 0 | 0 | 0.7 | 0 | 0 | 0 | 0 | 0 | 0 | 0 |
| 261 | 0 | 1 | 0 | 0 | 0 | 0 | 0 | 0 | 0 | 0 | 0 |
| 262 | 0 | 0 | 0 | 0 | 0 | 0 | 0 | 0 | 0 | 0 | 0 |
| 263 | 1 | 0 | 0 | 0 | 0 | 0 | 0 | 0 | 0 | 0 | 0 |
| 264 | 0.7 | 0 | 0 | 0 | 0 | 0 | 0 | 0 | 0 | 0 | 0 |
| 265 | 0 | 0 | 0 | 0 | 0 | 0.7 | 0 | 0 | 0 | 0 | 0 |
| 266 | 0 | 0 | 0 | 0 | 0 | 0 | 0 | 0 | 0 | 0 | 0 |
| 267 | 0 | 0 | 0 | 0 | 0 | 0 | 0 | 0 | 0 | 0 | 0.7 |
| 268 | 0 | 0 | 0 | 0.7 | 0 | 0 | 0 | 0 | 0 | 0 | 0 |
| 269 | 0 | 0 | 0 | 0 | 0 | 0.7 | 0 | 0 | 0 | 0 | 0 |
| 270 | 0 | 0 | 0 | 0 | 0 | 0.7 | 0 | 0 | 0 | 0 | 0 |
| 271 | 0 | 0 | 0 | 0 | 0 | 0 | 0 | 0 | 0 | 0 | 0 |
| 272 | 0 | 0 | 0 | 0 | 0 | 0 | 0 | 0 | 0 | 0 | 0 |
| 273 | 0 | 0 | 1 | 0 | 0 | 0 | 0 | 0 | 0 | 0 | 0 |
| 274 | 0 | 0 | 0 | 0 | 0 | 0 | 0 | 0 | 0 | 0 | 0 |
| 275 | 0 | 0 | 0 | 0 | 0 | 0 | 0 | 0 | 0 | 0 | 0 |
| 276 | 0 | 0 | 0 | 0 | 0 | 0 | 0 | 0 | 0 | 0 | 0.7 |
| 277 | 0 | 0 | 0 | 0 | 0 | 0 | 0 | 0 | 0 | 0 | 0 |
| 278 | 0 | 0 | 0 | 0 | 0 | 0 | 0 | 0 | 0 | 0 | 0 |
| 279 | 0 | 0 | 0 | 0 | 0 | 0 | 0 | 0.7 | 0 | 0 | 0 |
| 280 | 0 | 0 | 0 | 0 | 0 | 0 | 0 | 0 | 0 | 0 | 0 |
| 281 | 0 | 0 | 0 | 0 | 0 | 0 | 0 | 0.7 | 0 | 0 | 0 |
| 282 | 0 | 0 | 0 | 0 | 0 | 0 | 0 | 0 | 0 | 0 | 0 |
| 283 | 0 | 0 | 0 | 0 | 0 | 0.7 | 0 | 0 | 0 | 0 | 0 |
| 284 | 0 | 0 | 0 | 0 | 0 | 0 | 0 | 0 | 0 | 0 | 0 |
| 285 | 0 | 0 | 0 | 0 | 0 | 1 | 0.7 | 0 | 0 | 0 | 0 |
| 286 | 0 | 0 | 0 | 0 | 0 | 0 | 0 | 0 | 0 | 0 | 0.7 |
| 287 | 0 | 0 | 0 | 0.7 | 0 | 0 | 0 | 0 | 0 | 0 | 0 |
| 288 | 1 | 0 | 0 | 0 | 0 | 0 | 1 | 0 | 0 | 0 | 0 |
| 289 | 0.7 | 0 | 0 | 0 | 0 | 0 | 0 | 0 | 0 | 0 | 0 |
| 290 | 0 | 0 | 0 | 0.7 | 0 | 0 | 0 | 0 | 0 | 0 | 0 |
| 291 | 0 | 0 | 0 | 0 | 0 | 0 | 0 | 0 | 0 | 0 | 0 |
| 292 | 0 | 0 | 0 | 0 | 0 | 0 | 0 | 0 | 0 | 0 | 0.7 |
| 293 | 0 | 0 | 0 | 0 | 0 | 0 | 0.7 | 0 | 0 | 0 | 0 |
| 294 | 0 | 0 | 0 | 0 | 0 | 0 | 0 | 0 | 0 | 0.7 | 0 |
| 295 | 0.7 | 0 | 0 | 0 | 0 | 0 | 0 | 0 | 0 | 0 | 0 |
| 296 | 0 | 0.7 | 0 | 0 | 0 | 0 | 0 | 0 | 1 | 0 | 0 |
| 297 | 0 | 0 | 0 | 0 | 0 | 0 | 0 | 0.7 | 0 | 0 | 0 |
| 298 | 0 | 0 | 0 | 0 | 0 | 0 | 0 | 0 | 0 | 0 | 0.7 |
| 299 | 0 | 0 | 0 | 0 | 0 | 0 | 0 | 0 | 0 | 0 | 0 |
| 300 | 0.7 | 0 | 0 | 1 | 0 | 0 | 0 | 0 | 0 | 0 | 0 |
| 301 | 0 | 0 | 0 | 0 | 0 | 0 | 0 | 0 | 0 | 0 | 0 |
| 302 | 0 | 0 | 0 | 0 | 0 | 0 | 0 | 0 | 0 | 0 | 0 |
| 303 | 0 | 0 | 0 | 0 | 0 | 0.7 | 0 | 0 | 0 | 0 | 0 |
| 304 | 0 | 0 | 0 | 0 | 0 | 0 | 0 | 0 | 0 | 0 | 0 |
| 305 | 0 | 0 | 0 | 0 | 0 | 0 | 0 | 0 | 0 | 0 | 0 |
| 306 | 0 | 0 | 0 | 0 | 0 | 0 | 0 | 0 | 0 | 0 | 0.7 |
| 307 | 0 | 0 | 0 | 0 | 0 | 0 | 0 | 0 | 0 | 0 | 0 |
| 308 | 0 | 0 | 0 | 0 | 0 | 0 | 0 | 0 | 0 | 0 | 0.7 |
| 309 | 0 | 0 | 0 | 0 | 0 | 0 | 0 | 0 | 0 | 0 | 0 |
| 310 | 0 | 0.7 | 0 | 0 | 0 | 0 | 0 | 0 | 0 | 0 | 0 |
| 311 | 0 | 0 | 0 | 0 | 0 | 0 | 0 | 0 | 0 | 0 | 0 |
| 312 | 0 | 0 | 0 | 0 | 0 | 0 | 0 | 0.7 | 0 | 0 | 0 |
| 313 | 0 | 0 | 0 | 0 | 0 | 0 | 0 | 0 | 0 | 0 | 0 |
| 314 | 0 | 0 | 0 | 0 | 0 | 0 | 0 | 0 | 0 | 0 | 0 |
| 315 | 0 | 0 | 0 | 0 | 0 | 0 | 0 | 0 | 0 | 0 | 0 |
| 316 | 0 | 0 | 0 | 0 | 0 | 0 | 0 | 0.7 | 0 | 0 | 0 |
| 317 | 0 | 0 | 0 | 0 | 0 | 0 | 0 | 0 | 0 | 0 | 0 |
| 318 | 0 | 0 | 0 | 0 | 0 | 0 | 0.7 | 0 | 0 | 0 | 0 |
| 319 | 0 | 0 | 0 | 0 | 0 | 0 | 0 | 0 | 0 | 0 | 0 |
| 320 | 0 | 0 | 0 | 0 | 0 | 0 | 0 | 0 | 0 | 0.7 | 1 |
| 321 | 0 | 0 | 0 | 0 | 0 | 0 | 0.7 | 0 | 0 | 0 | 0 |
| 322 | 0 | 0 | 0 | 0 | 0 | 0 | 0 | 0 | 0 | 0 | 0.7 |
| 323 | 0 | 0 | 0 | 0 | 0 | 0 | 0 | 0 | 0 | 0 | 0.7 |
| 324 | 0 | 0 | 0 | 0 | 0 | 0 | 0 | 0 | 0 | 0 | 0 |
| 325 | 0 | 0 | 0 | 0.7 | 0 | 0 | 0 | 0 | 0 | 0 | 0 |
| 326 | 0 | 0 | 0 | 0 | 0 | 0 | 0 | 0.7 | 0 | 0 | 0 |
| 327 | 0 | 0 | 0 | 0 | 0 | 0 | 0 | 0 | 0 | 0 | 0 |
| 328 | 0 | 0 | 0 | 0 | 0 | 0 | 0 | 0 | 0 | 0 | 0.7 |
| 329 | 0 | 0 | 0 | 0 | 0 | 0 | 0 | 0.7 | 0 | 0 | 0 |
| 330 | 0 | 0 | 0 | 0 | 0 | 0 | 0.7 | 0 | 0 | 0 | 0 |
| 331 | 0 | 1 | 0 | 0 | 0 | 0 | 0 | 0 | 0 | 0 | 0 |
| 332 | 0 | 0 | 0 | 0 | 0 | 0 | 0 | 0.7 | 0 | 0 | 0 |
| 333 | 0 | 0.7 | 0 | 0 | 0 | 0 | 0 | 0 | 0 | 0 | 0 |
| 334 | 0 | 0 | 0 | 0 | 0 | 0 | 0 | 0 | 0 | 0.7 | 0 |
| 335 | 0 | 0 | 0 | 0 | 0 | 0 | 0 | 0 | 0 | 0 | 0 |
| 336 | 0 | 0 | 0 | 0 | 0 | 0 | 0 | 0 | 0 | 0 | 0 |
| 337 | 0 | 0 | 0 | 0 | 0 | 0 | 0 | 0 | 0 | 0 | 0 |
| 338 | 0.7 | 0 | 0 | 0 | 0.7 | 0 | 0 | 0 | 0 | 0 | 0 |
| 339 | 0 | 0 | 0 | 0 | 0 | 0 | 0 | 0 | 0 | 0 | 0 |
| 340 | 0 | 0 | 0 | 0 | 0 | 0.7 | 0 | 0 | 0 | 0 | 0 |
| 341 | 0 | 0 | 0 | 0 | 0 | 0.7 | 0 | 0 | 0 | 0 | 0 |
| 342 | 0 | 0 | 0 | 0 | 0 | 0 | 0.7 | 0 | 0 | 0 | 0 |
| 343 | 0 | 0 | 0 | 0 | 0 | 0 | 0 | 0 | 0 | 0 | 0 |
| 344 | 0 | 0 | 0 | 0 | 0 | 0 | 0 | 0 | 0 | 0 | 0.7 |
| 345 | 0.7 | 0 | 0 | 0 | 0 | 0 | 0 | 0 | 0 | 0 | 0 |
| 346 | 0 | 0 | 0 | 0 | 0 | 0 | 0 | 0 | 0.7 | 0 | 0 |
| 347 | 0 | 0 | 0 | 0 | 0 | 0 | 1 | 1 | 0 | 0 | 0 |
| 348 | 0.7 | 0 | 0 | 0 | 0 | 0 | 0 | 0 | 0 | 0 | 0 |
| 349 | 0 | 0 | 0 | 0 | 0 | 0 | 0 | 0 | 0 | 0.7 | 0 |
| 350 | 0 | 0 | 0 | 0 | 0 | 0 | 0 | 0.7 | 0 | 0 | 0 |
| 351 | 0.7 | 0 | 0 | 0 | 0 | 0 | 0 | 0 | 0 | 0 | 0 |
| 352 | 0 | 0 | 0 | 0 | 0 | 0 | 0 | 0 | 0 | 0 | 0 |
| 353 | 0 | 0 | 0 | 0 | 0 | 0 | 0 | 0 | 0 | 0 | 0 |
| 354 | 0 | 0 | 0 | 0 | 0 | 0 | 0 | 0 | 0 | 0 | 0 |
| 355 | 0 | 0 | 0 | 0 | 0 | 0 | 0 | 0 | 0.7 | 0 | 0 |
| 356 | 0 | 0 | 0 | 0 | 0 | 0 | 0 | 0.7 | 0 | 0 | 0 |
| 357 | 0 | 0 | 0 | 0 | 0 | 0 | 0 | 0.7 | 0 | 0 | 0 |
| 358 | 0 | 0 | 0 | 0 | 0 | 0 | 0 | 0 | 0 | 0 | 0 |
| 359 | 0 | 0 | 0.7 | 0 | 0 | 0 | 0 | 0 | 0 | 0 | 0 |
| 360 | 0 | 0 | 0 | 0 | 0 | 0 | 0 | 0.7 | 0 | 0 | 0 |
| 361 | 0 | 0 | 0 | 0 | 0 | 0 | 0 | 0 | 0 | 0 | 0 |
| 362 | 0 | 0 | 0 | 0 | 0 | 0 | 0 | 0 | 0 | 0 | 1 |
| 363 | 0 | 0 | 0 | 0 | 0 | 0 | 0.7 | 0 | 0 | 0 | 0 |
| 364 | 0.7 | 0 | 0 | 0 | 0 | 0 | 0 | 0 | 0 | 0 | 0 |
| 365 | 0 | 0 | 0 | 0 | 0 | 0 | 0 | 0 | 0 | 0 | 0 |
| 366 | 0 | 0 | 0 | 0 | 0.7 | 0 | 0 | 0 | 0 | 0 | 0 |
| 367 | 0 | 0 | 0 | 0 | 0 | 0 | 0.7 | 0 | 0 | 0 | 0 |
| 368 | 0 | 0 | 0 | 0 | 0 | 0 | 0 | 0.7 | 0 | 0 | 0 |
| 369 | 0 | 0 | 0 | 0 | 0 | 0 | 0 | 0 | 0 | 0 | 0 |
| 370 | 0 | 0 | 0 | 0 | 0 | 0 | 0 | 0 | 0 | 0 | 0 |
| 371 | 0 | 0 | 0 | 0 | 0 | 0 | 0 | 0 | 0 | 0 | 0 |
| 372 | 0 | 0 | 0 | 0 | 0 | 0 | 0 | 0 | 0 | 0.7 | 0 |
| 373 | 0 | 0 | 0 | 0 | 1 | 0 | 0 | 0 | 0 | 0 | 0 |
| 374 | 0 | 0 | 0 | 0 | 0 | 0 | 0 | 0 | 0 | 0 | 0 |
| 375 | 0 | 0 | 0 | 0.7 | 0 | 0 | 0 | 0 | 0 | 0 | 0 |
| 376 | 0 | 0 | 0 | 0 | 0 | 0 | 0 | 0 | 0 | 0 | 0 |
| 377 | 0 | 0 | 0 | 0 | 0 | 0 | 0 | 0.7 | 0 | 0 | 0 |
| 378 | 0 | 0 | 0 | 0 | 0 | 0 | 0 | 0 | 0 | 0 | 0.7 |
| 379 | 0 | 0 | 0 | 0.7 | 0 | 0 | 0 | 0 | 0 | 0 | 0 |
| 380 | 0 | 0 | 0 | 0 | 0 | 0.7 | 0 | 0 | 0 | 0 | 0 |
| 381 | 0 | 0 | 0 | 0 | 0 | 0 | 0 | 0 | 0 | 0 | 0 |
| 382 | 0 | 0 | 0 | 0 | 0 | 0 | 0 | 0 | 0 | 0 | 0.7 |
| 383 | 0 | 0 | 0.7 | 0 | 0 | 0 | 0 | 0 | 0 | 0 | 0 |
| 384 | 0.7 | 0 | 0 | 0 | 0 | 0 | 0 | 0 | 0 | 0 | 0 |
| 385 | 0 | 0.7 | 0 | 0 | 0 | 0 | 0 | 0 | 0 | 0 | 0 |
| 386 | 0 | 0 | 0 | 0 | 0 | 0 | 0 | 0 | 0 | 0 | 0 |
| 387 | 0 | 0 | 0 | 0 | 0 | 0 | 0 | 0 | 0 | 0 | 0 |
| 388 | 0 | 0 | 0 | 0 | 0 | 0 | 0 | 0 | 0 | 0 | 0 |
| 389 | 0 | 0 | 0 | 1 | 0 | 0 | 0.7 | 0 | 0 | 0 | 0 |
| 390 | 0 | 0 | 0 | 0 | 0 | 0 | 0 | 0 | 0 | 0 | 0 |
| 391 | 0 | 0 | 0 | 0 | 0 | 0 | 0 | 0 | 0 | 0 | 0.7 |
| 392 | 0 | 0 | 0 | 0 | 0 | 0 | 0 | 0 | 0.7 | 0 | 0 |
| 393 | 0 | 0 | 0 | 0 | 0 | 0 | 0 | 0 | 0 | 0 | 0 |
| 394 | 0 | 0 | 0 | 0 | 0 | 0 | 0 | 0.7 | 0 | 0 | 0 |
| 395 | 0 | 0 | 0 | 0 | 0 | 0 | 0 | 0 | 0 | 0 | 0 |
| 396 | 0 | 0 | 0 | 0 | 0 | 0 | 0 | 0 | 0 | 0 | 0 |
| 397 | 0 | 0 | 0 | 0 | 0 | 0 | 0 | 0 | 0 | 0 | 0.7 |
| 398 | 0 | 0 | 0 | 0 | 0 | 0 | 0.7 | 0 | 0 | 0 | 0 |
| 399 | 0 | 0 | 0.7 | 0 | 0 | 0 | 0 | 0 | 0 | 0 | 0 |
| 400 | 0 | 0 | 0 | 0 | 0 | 0 | 0 | 0.7 | 0 | 0 | 0 |
| 401 | 0 | 0 | 0 | 0 | 0 | 0 | 0 | 0 | 0 | 0 | 0 |
| 402 | 0 | 0 | 0 | 0 | 0 | 0 | 0.7 | 0 | 0 | 0.7 | 0 |
| 403 | 0 | 0.7 | 0 | 0 | 0 | 0 | 0 | 0 | 0 | 0 | 0 |
| 404 | 0 | 0 | 0 | 0 | 0 | 0 | 1 | 0.7 | 0 | 0 | 0 |
| 405 | 0 | 0 | 0 | 0 | 0 | 0 | 0.7 | 0 | 0 | 0 | 0 |
| 406 | 0 | 0 | 0 | 0 | 0 | 0 | 0 | 0 | 0 | 0.7 | 0 |
| 407 | 1 | 0 | 0 | 0 | 0 | 0 | 0 | 0 | 0 | 0.7 | 0 |
| 408 | 0 | 0 | 0.7 | 0 | 0 | 0 | 0 | 0 | 0 | 0 | 0 |
| 409 | 0.7 | 0 | 0 | 0 | 0 | 0 | 0 | 0 | 0 | 0 | 0 |
| 410 | 0 | 0 | 0 | 0 | 0 | 0 | 0 | 0 | 0 | 0.7 | 0 |
| 411 | 0 | 0.7 | 0 | 0 | 0 | 0 | 0 | 0 | 0 | 0 | 0 |
| 412 | 0 | 0.7 | 0 | 0 | 0 | 0 | 0 | 0 | 0 | 0 | 0 |
| 413 | 0 | 0 | 0 | 0 | 0 | 0 | 0 | 0 | 0 | 0 | 0 |
| 414 | 0 | 0 | 0 | 0 | 0 | 0 | 0 | 0 | 0 | 0 | 0 |
| 415 | 0 | 0 | 0 | 0 | 0 | 0 | 0 | 0 | 0 | 0 | 0 |
| 416 | 0 | 0 | 0 | 0.7 | 0 | 0 | 0 | 0 | 0 | 0 | 0 |
| 417 | 0 | 0 | 0 | 0 | 0 | 0 | 1 | 0 | 0 | 0 | 0 |
| 418 | 0 | 0 | 0 | 0 | 0 | 0 | 0.7 | 0 | 0 | 0 | 0 |
| 419 | 0 | 0 | 0 | 0 | 0 | 0 | 0 | 0.7 | 0 | 0 | 0 |
| 420 | 0 | 0 | 0 | 0 | 0 | 0 | 0.7 | 0 | 0 | 0 | 0 |
| 421 | 0 | 0 | 0 | 0 | 0 | 0 | 1 | 0 | 0 | 0 | 0 |
| 422 | 0 | 0 | 0 | 0 | 0 | 0 | 0 | 0 | 0 | 1 | 0 |
| 423 | 0 | 0.7 | 0 | 0 | 0 | 0 | 0 | 0 | 0 | 0 | 0 |
| 424 | 0 | 0 | 0 | 0 | 0 | 0.7 | 1 | 0 | 0 | 0 | 0 |
| 425 | 0 | 1 | 0.7 | 0 | 0 | 0 | 0 | 0 | 0 | 0 | 0 |
| 426 | 0.7 | 0 | 0 | 0 | 0 | 0 | 0 | 0 | 0 | 0 | 0 |
| 427 | 0 | 0.7 | 0 | 0 | 0 | 0 | 0 | 0 | 0 | 0 | 0 |
| 428 | 0 | 0 | 0 | 0 | 0 | 0 | 0 | 0 | 0 | 0 | 0 |
| 429 | 0 | 0 | 0 | 0 | 0 | 0 | 0 | 0 | 0 | 0 | 0.7 |
| 430 | 0 | 0 | 0 | 0 | 0 | 0 | 0 | 0 | 0 | 0 | 0 |
| 431 | 0 | 0 | 0 | 0.7 | 0 | 1 | 1 | 0 | 0 | 0 | 0 |
| 432 | 0 | 0 | 0 | 0 | 0 | 0 | 0 | 0 | 0 | 0 | 0 |
| 433 | 1 | 0 | 0 | 0 | 0 | 0 | 0 | 0 | 0 | 0 | 0.7 |
| 434 | 0 | 0 | 0 | 0.7 | 0 | 0 | 0 | 0 | 0 | 0 | 0 |
| 435 | 0 | 0.7 | 0 | 0 | 0 | 0 | 0 | 0 | 0 | 0 | 0 |
| 436 | 0 | 0 | 0 | 0 | 0 | 0 | 0 | 0 | 0 | 0 | 0 |
| 437 | 0 | 0 | 0 | 0 | 0 | 0 | 0 | 0 | 0 | 0.7 | 1 |
| 438 | 0.7 | 0 | 0 | 0 | 0 | 0 | 0 | 0 | 0 | 0 | 0 |
| 439 | 0 | 0 | 0 | 0 | 0 | 0 | 0 | 0 | 0 | 0 | 0 |
| 440 | 0 | 0 | 0 | 0 | 0 | 0 | 0 | 0.7 | 0 | 0 | 0 |
| 441 | 0 | 0 | 0 | 0 | 0 | 0 | 0 | 0.7 | 0 | 0 | 0 |
| 442 | 0 | 0 | 0 | 0 | 0 | 0 | 0 | 0 | 0 | 0 | 0 |
| 443 | 0 | 0 | 0 | 0 | 0 | 0 | 0 | 0 | 0 | 0 | 0 |
| 444 | 0 | 0 | 0 | 0 | 0 | 0 | 0 | 0 | 0 | 0 | 0 |
| 445 | 0 | 0 | 0 | 0 | 0 | 0 | 0.7 | 0 | 0 | 0 | 0 |
| 446 | 0 | 0 | 0 | 0 | 0 | 0 | 0 | 0 | 0 | 0 | 0 |
| 447 | 0 | 0 | 0 | 0 | 0 | 0 | 0 | 0 | 0 | 0 | 0.7 |
| 448 | 0 | 0 | 0 | 0 | 0 | 0 | 0.7 | 0 | 0 | 0 | 0 |
| 449 | 0 | 0.7 | 0 | 0 | 0 | 0 | 0 | 0 | 0 | 0 | 0 |
| 450 | 0 | 0 | 0 | 0 | 0 | 0 | 0 | 0 | 0 | 0 | 0.7 |
| 451 | 0 | 0 | 0 | 0 | 0 | 0.7 | 0 | 0 | 0 | 0 | 0 |
| 452 | 0 | 0 | 0 | 0 | 0 | 0 | 0 | 0 | 0 | 0 | 0 |
| 453 | 0 | 0 | 0 | 0 | 0 | 0 | 0 | 0 | 0 | 0 | 0 |
| 454 | 0 | 0 | 0 | 0 | 0 | 0 | 0 | 0 | 0 | 0.7 | 0 |
| 455 | 0 | 0 | 0 | 0 | 0 | 0 | 0 | 0 | 0 | 0 | 0 |
| 456 | 0 | 0 | 0 | 0 | 0 | 0 | 0 | 0 | 0 | 0 | 0 |
| 457 | 0 | 0 | 0 | 0 | 0 | 0 | 0 | 0 | 0 | 0 | 0.7 |
| | Seq | | | | | | | | | | |
| | Pos | K | M | F | P | S | T | W | Y | V | — |
| | 1 | 0 | 0.7 | 0 | 0 | 0 | 0 | 0 | 0 | 0 | 0 |
| | 2 | 0 | 0 | 0 | 0.7 | 0 | 0 | 0 | 0 | 0 | 0 |
| | 3 | 0 | 0 | 0 | 0 | 0 | 0 | 0 | 0 | 0 | 0 |
| | 4 | 0 | 0 | 0 | 0 | 0 | 0.7 | 0 | 0 | 0 | 0 |
| | 5 | 0 | 0 | 0 | 0 | 0.7 | 0 | 0 | 0 | 0 | 0 |
| | 6 | 0 | 0 | 0 | 0.7 | 0 | 1 | 0 | 0 | 0 | 0 |
| | 7 | 0 | 0 | 0 | 0 | 0 | 0.7 | 0 | 0 | 0 | 0 |
| | 8 | 0 | 0 | 0 | 0 | 0 | 0.7 | 0 | 0 | 1 | 0 |
| | 9 | 0 | 0 | 0 | 0 | 0 | 0 | 0 | 0 | 0.7 | 0 |
| | 10 | 0 | 0 | 0 | 0 | 0 | 0 | 0 | 0 | 0 | 0 |
| | 11 | 0 | 0 | 0 | 0 | 0 | 0 | 0 | 0 | 0 | 0 |
| | 12 | 0 | 0 | 0 | 0 | 0 | 0 | 0 | 0 | 0 | 0 |
| | 13 | 0 | 0 | 0 | 0 | 0 | 0 | 0 | 0 | 0 | 0 |
| | 14 | 0 | 0 | 0 | 0 | 0 | 0 | 0 | 0 | 0.7 | 0 |
| | 15 | 0 | 0 | 0 | 0 | 0 | 0 | 0 | 0 | 0 | 0 |
| | 16 | 0 | 0.7 | 0 | 0 | 0 | 0 | 0 | 0 | 0 | 0 |
| | 17 | 0 | 0 | 0.7 | 0 | 0 | 0 | 0 | 0 | 0 | 0 |
| | 18 | 0 | 0 | 0 | 0.7 | 0 | 0 | 0 | 0 | 0 | 0 |
| | 19 | 0 | 0 | 0 | 0 | 0 | 0 | 0 | 0 | 0.7 | 0 |
| | 20 | 0 | 0 | 0 | 0.7 | 0 | 0 | 0 | 0 | 0 | 0 |
| | 21 | 0 | 0 | 0.7 | 0 | 0 | 0 | 0 | 0 | 0 | 0 |
| | 22 | 0 | 0 | 0 | 0.7 | 0 | 0 | 0 | 0 | 0 | 0 |
| | 23 | 0 | 0 | 0 | 0 | 0 | 0 | 0 | 0 | 0 | 0 |
| | 24 | 0 | 0 | 0 | 0 | 0 | 0 | 0 | 0 | 0 | 0 |
| | 25 | 0 | 0 | 0 | 0 | 0 | 0 | 0 | 0 | 1 | 0 |
| | 26 | 0 | 0 | 0 | 0 | 0 | 0 | 0 | 0 | 0 | 0 |
| | 27 | 0 | 0 | 0 | 0.7 | 0 | 0 | 0 | 0 | 0 | 0 |
| | 28 | 0 | 0.7 | 0 | 0 | 0 | 0 | 0 | 0 | 0 | 0 |
| | 29 | 0 | 0 | 0 | 0 | 0 | 0 | 0 | 0 | 0 | 0 |
| | 30 | 0 | 0 | 0 | 0 | 0 | 0 | 0 | 0 | 0 | 0 |
| | 31 | 0 | 0 | 0 | 0 | 0 | 0 | 0 | 0 | 0 | 0 |
| | 32 | 0 | 0 | 0 | 0 | 0 | 0 | 0 | 0 | 0 | 0 |
| | 33 | 0 | 0 | 0 | 0 | 0 | 0 | 0 | 0 | 0 | 0 |
| | 34 | 0 | 0 | 0 | 0 | 0 | 0 | 0 | 0 | 0.7 | 0 |
| | 35 | 0 | 0 | 0 | 0 | 0 | 0 | 0 | 0 | 0 | 0 |
| | 36 | 0 | 0 | 0 | 0 | 0 | 0 | 0 | 0.7 | 0 | 0 |
| | 37 | 0 | 0 | 0 | 0 | 1 | 0 | 0 | 0 | 0 | 0 |
| | 38 | 1 | 0 | 0 | 0 | 0 | 0 | 0 | 0 | 0 | 0 |
| | 39 | 0 | 0 | 0 | 0 | 0 | 0 | 0 | 0 | 0 | 0 |
| | 40 | 0 | 0 | 0.7 | 0 | 0 | 0 | 0 | 0 | 0 | 0 |
| | 41 | 0 | 0 | 0 | 0 | 1 | 0 | 0 | 0 | 0 | 0 |
| | 42 | 0 | 0 | 0 | 0 | 0 | 0 | 0 | 0 | 0 | 0 |
| | 43 | 0 | 0 | 0 | 0 | 0 | 0.7 | 0 | 0 | 0 | 0 |
| | 44 | 0 | 0 | 0 | 0 | 0 | 0 | 0 | 0 | 0 | 0 |
| | 45 | 0 | 0 | 1 | 0 | 0 | 0 | 0 | 0 | 0 | 0 |
| | 46 | 0 | 0 | 0 | 0 | 0 | 0 | 0 | 0 | 0 | 0 |
| | 47 | 0 | 0 | 0 | 0 | 0 | 0.7 | 0 | 0 | 0 | 0 |
| | 48 | 0 | 0 | 0 | 0 | 0 | 0 | 0 | 0 | 0 | 0 |
| | 49 | 0 | 0 | 0.7 | 0 | 0 | 0 | 0 | 0 | 0 | 0 |
| | 50 | 0 | 0 | 0 | 0 | 0 | 0 | 0 | 0 | 0 | 0 |
| | 51 | 1 | 0 | 0 | 0 | 0 | 0 | 0 | 0 | 0 | 0 |
| | 52 | 0 | 0 | 0 | 0.7 | 0 | 0 | 0 | 0 | 0 | 0 |
| | 53 | 0.7 | 0 | 0 | 0 | 0 | 0 | 0 | 0 | 0 | 0 |
| | 54 | 0 | 0 | 0 | 0 | 0 | 0.7 | 0 | 0 | 0 | 0 |
| | 55 | 0 | 0 | 0 | 0 | 0.7 | 0 | 0 | 0 | 0 | 0 |
| | 56 | 0 | 0 | 0 | 0 | 0 | 0 | 0 | 0 | 0 | 0 |
| | 57 | 0 | 0 | 0 | 0 | 0 | 0 | 0 | 0.7 | 0 | 0 |
| | 58 | 0 | 0 | 0 | 0.7 | 0 | 0 | 0 | 0 | 0 | 0 |
| | 59 | 0 | 0 | 0 | 0 | 0 | 0 | 0 | 0 | 0 | 0 |
| | 60 | 0 | 0 | 0.7 | 0 | 0 | 0 | 0 | 0 | 0 | 0 |
| | 61 | 0 | 0 | 0 | 0 | 0 | 1 | 0 | 0 | 0 | 0 |
| | 62 | 0 | 0 | 0.7 | 0 | 0 | 0 | 0 | 0 | 0 | 0 |
| | 63 | 0 | 0 | 0 | 0 | 0 | 0 | 0 | 0 | 0 | 0 |
| | 64 | 0 | 0 | 0.7 | 0 | 0 | 0 | 0 | 0 | 0 | 0 |
| | 65 | 0 | 0 | 0 | 0 | 0 | 0 | 0 | 0 | 0 | 0 |
| | 66 | 0 | 0 | 0 | 0 | 0 | 0 | 0 | 0 | 0 | 0 |
| | 67 | 0 | 0 | 0 | 0 | 0 | 0 | 0 | 0 | 0 | 0 |
| | 68 | 0 | 0 | 0 | 0 | 0 | 0 | 0 | 0 | 0 | 0 |
| | 69 | 0 | 0 | 0 | 0 | 0 | 0 | 0 | 0 | 0 | 0 |
| | 70 | 0 | 0 | 0 | 0.7 | 0 | 0 | 0 | 0 | 0 | 0 |
| | 71 | 0 | 0 | 0 | 0 | 0 | 0 | 0 | 0 | 0 | 0 |
| | 72 | 0 | 0 | 0 | 0.7 | 0 | 0.7 | 0 | 0 | 0 | 0 |
| | 73 | 0 | 0 | 0 | 0 | 0 | 0 | 0 | 0 | 0 | 0 |
| | 74 | 0 | 0 | 0 | 0 | 0 | 0 | 0.7 | 0 | 0 | 0 |
| | 75 | 0 | 0 | 0 | 0 | 0 | 0 | 0 | 0 | 0 | 0 |
| | 76 | 0 | 0 | 0 | 0 | 0 | 0 | 0 | 0 | 0 | 0 |
| | 77 | 0 | 0 | 0 | 0 | 0 | 0 | 0 | 0 | 0 | 0 |
| | 78 | 0 | 0 | 0 | 0 | 0 | 0 | 0 | 0 | 0 | 0 |
| | 79 | 0 | 0 | 0 | 0.7 | 0 | 0 | 0 | 0 | 0 | 0 |
| | 80 | 0 | 0 | 0 | 0 | 0 | 0.7 | 0 | 0 | 0 | 0 |
| | 81 | 0 | 0 | 0 | 0 | 0 | 0.7 | 0 | 0 | 0 | 0 |
| | 82 | 0 | 0 | 0 | 0 | 0 | 0 | 0 | 0 | 0 | 0 |
| | 83 | 0 | 0 | 0 | 0.7 | 0 | 0 | 0 | 0 | 0 | 0 |
| | 84 | 0 | 0 | 0 | 0 | 0 | 0 | 0 | 0 | 0 | 0 |
| | 85 | 0 | 0 | 0 | 0 | 0 | 0 | 0 | 0 | 0.7 | 0 |
| | 86 | 0 | 0 | 0 | 0 | 0 | 0 | 0 | 0 | 0 | 0 |
| | 87 | 0 | 0 | 0 | 0 | 0 | 0 | 0 | 0 | 0 | 0 |
| | 88 | 0 | 0 | 0 | 0 | 0 | 0 | 0 | 0 | 0 | 0 |
| | 89 | 0 | 0 | 0 | 0 | 0 | 0 | 0 | 0 | 0 | 0 |
| | 90 | 0 | 0 | 0 | 0.7 | 0 | 0 | 0 | 0 | 0 | 0 |
| | 91 | 0 | 0 | 0 | 0 | 0 | 0 | 0 | 0 | 0.7 | 0 |
| | 92 | 0 | 0 | 0 | 0 | 0 | 0 | 0 | 0 | 0 | 0 |
| | 93 | 0 | 0 | 0 | 0 | 0 | 0 | 0 | 0 | 0 | 0 |
| | 94 | 0.7 | 0 | 0 | 0 | 0 | 0 | 0 | 0 | 0 | 0 |
| | 95 | 0 | 0 | 0 | 0 | 0 | 0 | 0 | 0 | 0 | 0 |
| | 96 | 0 | 0 | 0 | 0 | 0 | 0 | 0 | 0 | 0 | 0 |
| | 97 | 0 | 0 | 0 | 0 | 0 | 0 | 0 | 0 | 0 | 0 |
| | 98 | 0 | 0 | 0 | 0 | 0 | 0 | 0 | 0 | 0 | 0 |
| | 99 | 0 | 0 | 0 | 0 | 0 | 0 | 0 | 0 | 0 | 0 |
| | 100 | 0 | 0 | 0.7 | 0 | 0 | 0 | 0 | 0 | 0 | 0 |
| | 101 | 0 | 0 | 0 | 0 | 0 | 0 | 0 | 0 | 0 | 0 |
| | 102 | 0.7 | 0 | 0 | 0 | 0 | 0 | 0 | 0 | 0 | 0 |
| | 103 | 0 | 0 | 0 | 0 | 0 | 0 | 0 | 0 | 0 | 0 |
| | 104 | 0 | 0 | 0 | 0 | 0 | 0 | 0 | 0 | 0 | 0 |
| | 105 | 0 | 0 | 0 | 0 | 0 | 0 | 0 | 0 | 0 | 0 |
| | 106 | 1 | 0 | 0 | 0 | 0.7 | 0 | 0 | 0 | 0 | 0 |
| | 107 | 0 | 0 | 0 | 0 | 0 | 0 | 0 | 0 | 0 | 0 |
| | 108 | 0 | 0.7 | 0 | 0 | 0 | 0 | 0 | 0 | 0 | 0 |
| | 109 | 0 | 0 | 0 | 0 | 0 | 0 | 0 | 0 | 0 | 0 |
| | 110 | 0 | 0 | 0 | 0 | 1 | 0 | 0 | 0 | 0 | 0 |
| | 111 | 0 | 0 | 0 | 0 | 0.7 | 0.7 | 0 | 0 | 0 | 0 |
| | 112 | 0 | 0 | 0 | 0.7 | 0 | 0 | 0 | 0 | 0 | 0 |
| | 113 | 0 | 0 | 0 | 0 | 0.7 | 0 | 0 | 0 | 0 | 0 |
| | 114 | 0 | 0 | 0 | 0 | 0 | 0 | 0 | 0 | 0 | 0 |
| | 115 | 0 | 0 | 0 | 0 | 0 | 0 | 0 | 0 | 0 | 0 |
| | 116 | 0 | 0 | 0 | 1 | 0 | 0 | 0 | 0 | 0 | 0 |
| | 117 | 0 | 0 | 0 | 0 | 0 | 0 | 0 | 0 | 0.7 | 0 |
| | 118 | 0 | 0 | 0 | 0 | 1 | 0 | 0 | 0 | 0 | 0 |
| | 119 | 0 | 0 | 0 | 0 | 0 | 0 | 0 | 0 | 0 | 0 |
| | 120 | 0 | 0 | 0 | 0 | 0 | 0 | 0 | 0 | 0.7 | 0 |
| | 121 | 0 | 0 | 0 | 0 | 0 | 0 | 0 | 0 | 0 | 0 |
| | 122 | 0 | 0 | 0 | 0 | 0 | 0.7 | 0 | 0 | 0 | 0 |
| | 123 | 0 | 0 | 0 | 0 | 0 | 0 | 0 | 0 | 0 | 0 |
| | 124 | 0 | 0 | 0 | 0 | 0 | 0 | 0 | 0 | 0 | 0 |
| | 125 | 0 | 0 | 0 | 0 | 0 | 0 | 0 | 0 | 0 | 0 |
| | 126 | 0 | 0 | 0 | 0 | 0 | 0 | 0.7 | 0 | 0 | 0 |
| | 127 | 0 | 0 | 0 | 0 | 0 | 0 | 0 | 0.7 | 0 | 0 |
| | 128 | 0 | 0 | 0.7 | 0 | 0 | 0 | 0 | 0 | 0 | 0 |
| | 129 | 0 | 0 | 0 | 0 | 0 | 1 | 0 | 0 | 0 | 0 |
| | 130 | 0 | 0 | 0 | 0 | 0 | 0 | 0 | 0 | 0 | 0 |
| | 131 | 0 | 0 | 0 | 0.7 | 1 | 0 | 0 | 0 | 0 | 0 |
| | 132 | 0 | 0 | 0 | 0 | 0 | 0 | 0 | 0 | 0.7 | 0 |
| | 133 | 0 | 0 | 0 | 0 | 0 | 0 | 0 | 0 | 0 | 0 |
| | 134 | 0 | 0 | 0 | 0 | 0 | 0 | 0 | 0 | 0 | 0 |
| | 135 | 0 | 0 | 0 | 0 | 0.7 | 0 | 0 | 0 | 0 | 0 |
| | 136 | 0 | 0 | 0 | 0 | 0 | 0 | 0 | 0 | 0 | 0 |
| | 137 | 0 | 0 | 0 | 0 | 0 | 0 | 0 | 0 | 0 | 0 |
| | 138 | 0 | 0 | 0 | 0 | 0 | 0 | 0 | 0 | 0 | 0 |
| | 139 | 0.7 | 0 | 0 | 1 | 0 | 0 | 0 | 0 | 0 | 0 |
| | 140 | 0 | 0 | 0 | 0 | 0 | 0 | 0 | 0 | 0 | 0 |
| | 141 | 0 | 0 | 0 | 0 | 0 | 0 | 0 | 0 | 0 | 0 |
| | 142 | 0 | 0 | 0 | 0 | 0 | 0 | 0 | 0 | 0.7 | 0 |
| | 143 | 0 | 0 | 0 | 0 | 0 | 0 | 0 | 0 | 0 | 0 |
| | 144 | 0 | 0.7 | 0 | 0 | 0 | 0 | 0 | 0 | 0 | 0 |
| | 145 | 0 | 0 | 0 | 0 | 0 | 0.7 | 0 | 0 | 0 | 0 |
| | 146 | 0 | 0 | 0 | 0 | 0.7 | 0 | 0 | 0 | 0 | 0 |
| | 147 | 0 | 0 | 0 | 0 | 0.7 | 0 | 0 | 0 | 0 | 0 |
| | 148 | 0 | 0 | 0 | 0 | 0 | 0 | 0 | 0 | 0 | 0 |
| | 149 | 0 | 0 | 0.7 | 0 | 0 | 0 | 0 | 0 | 0 | 0 |
| | 150 | 0 | 0 | 0 | 0 | 0 | 0 | 0 | 0 | 0 | 0 |
| | 151 | 0 | 0 | 0.7 | 0 | 0 | 0 | 0 | 0 | 0 | 0 |
| | 152 | 0 | 0 | 0 | 0 | 0 | 0 | 0 | 0 | 0 | 0 |
| | 153 | 0 | 0 | 0 | 0 | 0 | 0 | 0 | 0 | 0 | 0 |
| | 154 | 0 | 0 | 0 | 0 | 0 | 0 | 0 | 0 | 0 | 0 |
| | 155 | 0 | 0 | 0 | 0 | 0 | 0 | 0 | 0 | 0.7 | 0 |
| | 156 | 0 | 0 | 0 | 0 | 1 | 0 | 0 | 0 | 0 | 0 |
| | 157 | 0 | 0 | 0 | 0 | 0 | 0 | 0 | 0 | 0 | 0 |
| | 158 | 0 | 0 | 0 | 0.7 | 0 | 0 | 0 | 0 | 0 | 0 |
| | 159 | 0.7 | 0 | 0 | 0 | 0 | 0 | 0 | 0 | 0 | 0 |
| | 160 | 0 | 0 | 0.7 | 0 | 0 | 0 | 0 | 0 | 0 | 0 |
| | 161 | 0 | 0 | 0 | 0 | 0 | 0 | 0 | 0 | 0 | 0 |
| | 162 | 0 | 0 | 0 | 0 | 0 | 0 | 0 | 0 | 0 | 0 |
| | 163 | 0 | 0 | 0 | 0 | 0 | 0 | 0 | 0 | 0 | 0 |
| | 164 | 0 | 0 | 0 | 0 | 0 | 0 | 0 | 0 | 0 | 0 |
| | 165 | 0 | 0 | 0 | 0 | 0 | 0 | 0 | 0.7 | 0 | 0 |
| | 166 | 0 | 0 | 0 | 0 | 0 | 0 | 0 | 0 | 0 | 0 |
| | 167 | 0 | 0 | 0 | 0 | 0 | 0 | 0 | 0 | 0 | 0 |
| | 168 | 0 | 0 | 0 | 0.7 | 0 | 0 | 0 | 0 | 0 | 0 |
| | 169 | 0 | 0 | 0 | 0 | 0 | 0 | 0 | 0 | 0 | 0 |
| | 170 | 0 | 0 | 0 | 0 | 0 | 0.7 | 0 | 0 | 0 | 0 |
| | 171 | 0.7 | 0 | 0 | 0 | 0 | 0 | 0 | 0 | 0 | 0 |
| | 172 | 0 | 0 | 0 | 0 | 0 | 1 | 0 | 0 | 0 | 0 |
| | 173 | 0 | 0 | 0 | 0 | 0 | 0 | 0 | 0 | 0 | 0 |
| | 174 | 0 | 0 | 0 | 0.7 | 0 | 0 | 0 | 0 | 0 | 0 |
| | 175 | 0 | 0 | 0 | 0 | 0 | 0 | 0 | 0 | 0 | 0 |
| | 176 | 0 | 0 | 0 | 0 | 0 | 0 | 0 | 0 | 0 | 0 |
| | 177 | 0 | 0 | 0 | 0.7 | 0 | 0 | 0 | 0 | 0 | 0 |
| | 178 | 0 | 0 | 0 | 0 | 0 | 0 | 0 | 0 | 0.7 | 0 |
| | 179 | 0 | 0 | 0 | 0 | 1 | 0 | 0 | 0 | 0.7 | 0 |
| | 180 | 0 | 0 | 0 | 0 | 0 | 0 | 0 | 0 | 0 | 0 |
| | 181 | 0 | 0 | 0.7 | 0 | 0 | 0 | 0 | 0 | 0 | 0 |
| | 182 | 0 | 0 | 0 | 0.7 | 0 | 0 | 0 | 0 | 0 | 0 |
| | 183 | 0 | 0.7 | 0 | 0 | 0 | 0 | 0 | 0 | 0 | 0 |
| | 184 | 0 | 0 | 0 | 0 | 0 | 0 | 0 | 0 | 0 | 0 |
| | 185 | 0.7 | 0 | 0 | 0 | 0 | 0 | 0 | 0 | 0 | 0 |
| | 186 | 0 | 0 | 0 | 0 | 0 | 0 | 0 | 0 | 0.7 | 0 |
| | 187 | 0.7 | 0 | 0 | 0 | 0 | 0 | 0 | 0 | 0 | 0 |
| | 188 | 0 | 0 | 0 | 0 | 0 | 0 | 0 | 0 | 0 | 0 |
| | 189 | 0 | 0 | 0 | 0 | 0 | 0 | 0 | 0 | 0 | 0 |
| | 190 | 1 | 0 | 0 | 0 | 0 | 0 | 0 | 0 | 0 | 0 |
| | 191 | 1 | 0 | 0 | 0 | 1 | 0 | 0 | 0 | 0 | 0 |
| | 192 | 0 | 0 | 0 | 0 | 0 | 0 | 0 | 0 | 0 | 0 |
| | 193 | 0 | 0 | 0 | 0 | 0 | 0 | 0 | 0.7 | 0 | 0 |
| | 194 | 0 | 0 | 0 | 0 | 0.7 | 0 | 0 | 0 | 0 | 0 |
| | 195 | 0 | 0 | 0 | 0 | 0 | 0 | 0 | 0 | 0 | 0 |
| | 196 | 1 | 0 | 0 | 0 | 1 | 0 | 0.7 | 0 | 0 | 0 |
| | 197 | 0 | 0 | 0 | 0 | 0 | 0 | 0 | 0 | 0 | 0 |
| | 198 | 0 | 0 | 0 | 0 | 0 | 0 | 0 | 0 | 0 | 0 |
| | 199 | 0 | 0 | 0 | 0 | 0.7 | 0 | 0 | 0 | 0 | 0 |
| | 200 | 0.7 | 0 | 0 | 0 | 0 | 0 | 0 | 0 | 0 | 0 |
| | 201 | 0 | 0 | 0 | 0.7 | 0 | 0 | 0 | 0 | 0 | 0 |
| | 202 | 0 | 0 | 0 | 0 | 0 | 0 | 0 | 0 | 0 | 0 |
| | 203 | 0 | 0 | 0 | 0 | 0 | 0 | 0 | 0 | 0 | 0 |
| | 204 | 0 | 0.7 | 0 | 0 | 0 | 0 | 0 | 0 | 0 | 0 |
| | 205 | 0.7 | 0 | 0 | 0 | 0 | 0 | 0 | 0 | 0 | 0 |
| | 206 | 0 | 0.7 | 0 | 0 | 0 | 0 | 0 | 0 | 0 | 0 |
| | 207 | 0 | 0 | 0 | 0 | 0 | 0 | 0 | 0 | 0.7 | 0 |
| | 208 | 1 | 0 | 0 | 0 | 0 | 0 | 0 | 0 | 0 | 0 |
| | 209 | 0 | 0 | 0 | 0 | 0 | 0 | 0 | 0 | 0 | 0 |
| | 210 | 0 | 0 | 0 | 0 | 0 | 0.7 | 0 | 0 | 0 | 0 |
| | 211 | 1 | 0 | 0 | 0 | 0 | 0 | 0 | 0 | 0 | 0 |
| | 212 | 0 | 0 | 0 | 0 | 0 | 0 | 0 | 0 | 0 | 0 |
| | 213 | 0 | 0 | 0 | 0 | 0.7 | 0 | 0 | 0 | 0 | 0 |
| | 214 | 0 | 0 | 0 | 0 | 0.7 | 0 | 0 | 0 | 0 | 0 |
| | 215 | 0 | 0 | 0 | 0 | 0 | 0 | 0 | 0 | 0 | 0 |
| | 216 | 0 | 0 | 0 | 0 | 0 | 0 | 0 | 0 | 0.7 | 0 |
| | 217 | 0 | 0 | 0 | 0 | 0 | 0 | 0 | 0 | 0 | 0 |
| | 218 | 0 | 0 | 0 | 0 | 0 | 0 | 0.7 | 0 | 0 | 0 |
| | 219 | 0 | 0 | 0 | 0 | 0 | 0 | 0 | 0 | 0 | 0 |
| | 220 | 0 | 0 | 0 | 0 | 0.7 | 0 | 0 | 0 | 0 | 0 |
| | 221 | 0 | 0 | 0.7 | 0 | 0 | 0 | 0 | 0 | 0 | 0 |
| | 222 | 0.7 | 0 | 0 | 0 | 0 | 0 | 0 | 0 | 0 | 0 |
| | 223 | 0 | 0 | 0 | 0 | 0 | 0 | 0 | 0 | 0 | 0 |
| | 224 | 0 | 0 | 0 | 0 | 0 | 0 | 0 | 0 | 0 | 0 |
| | 225 | 0 | 0 | 0 | 0 | 0 | 0 | 0 | 0 | 0 | 0 |
| | 226 | 0 | 0 | 0 | 0 | 0 | 0 | 0 | 0 | 0 | 0 |
| | 227 | 0 | 0 | 0 | 0 | 0.7 | 0 | 0 | 0 | 0 | 0 |
| | 228 | 0 | 0 | 0 | 0 | 0 | 0 | 0 | 0 | 0 | 0 |
| | 229 | 0 | 0 | 0 | 0 | 0 | 0 | 0 | 0 | 0 | 0 |
| | 230 | 0 | 0 | 0 | 0 | 0 | 0 | 0 | 0 | 0 | 0 |
| | 231 | 0 | 0 | 0 | 0 | 0 | 0.7 | 0 | 0 | 0 | 0 |
| | 232 | 0 | 0 | 0 | 0 | 0 | 0 | 0 | 0 | 0.7 | 0 |
| | 233 | 0 | 0 | 0 | 0 | 0 | 0 | 0.7 | 0 | 0 | 0 |
| | 234 | 0 | 0 | 0 | 0 | 0 | 0 | 0 | 0 | 0 | 0 |
| | 235 | 0 | 0 | 0 | 0 | 0 | 0 | 0 | 0 | 0 | 0 |
| | 236 | 0 | 0 | 0 | 0 | 0 | 0 | 0 | 0 | 0 | 0 |
| | 237 | 0 | 0 | 0 | 0.7 | 0 | 0 | 0 | 0 | 0 | 0 |
| | 238 | 0 | 0 | 0 | 0 | 0 | 0 | 0 | 0 | 0 | 0 |
| | 239 | 0 | 0 | 0 | 0.7 | 0 | 0 | 0 | 0 | 0 | 0 |
| | 240 | 0 | 0 | 0 | 0 | 0.7 | 0 | 0 | 0 | 0 | 0 |
| | 241 | 0 | 0 | 0.7 | 0 | 0 | 0 | 0 | 0 | 0 | 0 |
| | 242 | 0 | 0 | 0 | 0 | 0 | 0 | 0 | 0 | 0 | 0 |
| | 243 | 0 | 0 | 0 | 0 | 0 | 0 | 0 | 0 | 0 | 0 |
| | 244 | 0 | 0 | 0 | 0.7 | 0 | 0 | 0 | 0 | 0 | 0 |
| | 245 | 0 | 0 | 0 | 0 | 0 | 0 | 0 | 0 | 0 | 0 |
| | 246 | 0 | 0 | 0 | 0.7 | 0 | 0 | 0 | 0 | 0 | 0 |
| | 247 | 0.7 | 0 | 0 | 0 | 0 | 0 | 0 | 0 | 0 | 0 |
| | 248 | 0 | 0 | 0 | 0 | 0 | 0 | 0 | 0 | 0 | 0 |
| | 249 | 0 | 0 | 1 | 0 | 0 | 0 | 0 | 0.7 | 0 | 0 |
| | 250 | 0 | 0 | 0 | 0 | 0 | 1 | 0 | 0 | 0 | 0 |
| | 251 | 0 | 0 | 0 | 0 | 0 | 0 | 0 | 0 | 0 | 0 |
| | 252 | 0 | 0 | 0 | 0 | 0.7 | 0 | 0 | 0 | 0 | 0 |
| | 253 | 0 | 0 | 0 | 0 | 0.7 | 0 | 0 | 0 | 0 | 0 |
| | 254 | 0 | 0 | 0 | 0 | 0.7 | 0.7 | 0 | 0 | 0 | 0 |
| | 255 | 0 | 0 | 0 | 0 | 0.7 | 0 | 0 | 0 | 0 | 0 |
| | 256 | 0 | 0 | 0 | 0 | 0 | 0 | 0 | 0 | 0 | 0 |
| | 257 | 0 | 0 | 0 | 0 | 0 | 0 | 0 | 0 | 0 | 0 |
| | 258 | 0 | 0 | 0 | 0 | 0 | 0 | 0 | 0 | 0 | 0 |
| | 259 | 0 | 0 | 0 | 0 | 0 | 0.7 | 0 | 0 | 0 | 0 |
| | 260 | 0 | 0 | 0 | 0 | 0 | 0 | 0 | 0 | 0 | 0 |
| | 261 | 0 | 0 | 0 | 0.7 | 0 | 0 | 0 | 0 | 0 | 0 |
| | 262 | 0 | 0 | 0 | 0 | 0.7 | 0 | 0 | 0 | 0 | 0 |
| | 263 | 0 | 0 | 1 | 0 | 0 | 0.7 | 0 | 0 | 1 | 0 |
| | 264 | 0 | 0 | 1 | 0 | 0 | 0 | 0 | 0 | 0 | 0 |
| | 265 | 0 | 0 | 0 | 1 | 0 | 0 | 0 | 0 | 0 | 0 |
| | 266 | 0 | 0 | 0 | 0 | 0 | 0 | 0.7 | 0 | 0 | 0 |
| | 267 | 0 | 0 | 0 | 0 | 0 | 0 | 0 | 0 | 0 | 0 |
| | 268 | 0 | 0 | 0 | 0 | 0 | 0 | 0 | 0 | 0 | 0 |
| | 269 | 0 | 0 | 0 | 0 | 0 | 0 | 0 | 0 | 0 | 0 |
| | 270 | 0 | 0 | 0 | 0 | 0 | 0 | 0 | 0 | 0 | 0 |
| | 271 | 0 | 0 | 0 | 0.7 | 0 | 0 | 0 | 0 | 0 | 0 |
| | 272 | 0 | 0 | 0 | 0.7 | 0 | 0 | 0 | 0 | 0 | 0 |
| | 273 | 1 | 0 | 0 | 0 | 0.7 | 0 | 0 | 0 | 0 | 0 |
| | 274 | 0 | 0 | 0 | 0 | 0.7 | 0 | 0 | 0 | 0 | 0 |
| | 275 | 0 | 0 | 0 | 0 | 0 | 0 | 0 | 0 | 0.7 | 0 |
| | 276 | 0 | 0 | 0 | 0 | 0 | 0 | 0 | 0 | 0 | 0 |
| | 277 | 0 | 0 | 0 | 0 | 0 | 0 | 0 | 0.7 | 0 | 0 |
| | 278 | 0 | 0 | 0 | 0 | 0 | 0 | 0 | 0 | 0.7 | 0 |
| | 279 | 0 | 0 | 0 | 0 | 1 | 0 | 0 | 0 | 0 | 0 |
| | 280 | 0 | 0 | 0.7 | 0 | 0 | 0 | 0 | 0 | 0 | 0 |
| | 281 | 0 | 0 | 0 | 0 | 0 | 0 | 0 | 0 | 0 | 0 |
| | 282 | 0 | 0 | 0 | 0 | 0.7 | 0 | 0 | 0 | 0 | 0 |
| | 283 | 0 | 0 | 0 | 0 | 0 | 0 | 0 | 0 | 0 | 0 |
| | 284 | 0 | 0 | 0 | 0 | 0.7 | 0 | 0 | 0 | 0 | 0 |
| | 285 | 0 | 0 | 0 | 0 | 0.7 | 0 | 0 | 0 | 0 | 0 |
| | 286 | 0 | 0 | 0 | 0 | 0 | 0 | 0 | 0 | 0.7 | 0 |
| | 287 | 0 | 0 | 0 | 0 | 0 | 0 | 0 | 0 | 0 | 0 |
| | 288 | 0 | 0 | 0 | 0.7 | 0 | 0 | 0 | 0 | 0 | 0 |
| | 289 | 0.7 | 0 | 0 | 0 | 0 | 0 | 0 | 0 | 0 | 0 |
| | 290 | 0 | 0 | 0 | 0 | 0 | 0 | 0 | 0 | 0 | 0 |
| | 291 | 0 | 0 | 0.7 | 0 | 0 | 0 | 0 | 0 | 0 | 0 |
| | 292 | 0 | 0 | 0 | 0 | 0 | 0 | 0 | 0 | 0 | 0 |
| | 293 | 0 | 0 | 0 | 0 | 0 | 0 | 0 | 0 | 0 | 0 |
| | 294 | 0 | 0 | 0 | 0 | 0 | 0 | 0 | 0 | 0 | 0 |
| | 295 | 0 | 0 | 0 | 0 | 0 | 0 | 0 | 0 | 0 | 0 |
| | 296 | 0 | 0 | 0 | 0 | 0 | 0 | 0 | 0 | 0 | 0 |
| | 297 | 0 | 0 | 0 | 0 | 0 | 0 | 0 | 0 | 0 | 0 |
| | 298 | 0 | 0 | 0 | 0 | 0 | 0 | 0 | 0 | 0 | 0 |
| | 299 | 0 | 0 | 0 | 0 | 0 | 0 | 0 | 0 | 0.7 | 0 |
| | 300 | 0 | 0 | 0 | 0 | 0 | 0 | 0 | 0 | 0 | 0 |
| | 301 | 0 | 0 | 0 | 0 | 0.7 | 0 | 0 | 0 | 0 | 0 |
| | 302 | 0.7 | 0 | 0 | 0 | 0 | 0 | 0 | 0 | 0 | 0 |
| | 303 | 0 | 0 | 0 | 0 | 0 | 0 | 0 | 0 | 0 | 0 |
| | 304 | 0 | 0 | 0 | 0 | 0.7 | 0 | 0 | 0 | 0 | 0 |
| | 305 | 0 | 0 | 0.7 | 0 | 0 | 0 | 0 | 0 | 0 | 0 |
| | 306 | 0 | 0 | 0 | 0 | 0 | 0 | 0 | 0 | 0 | 0 |
| | 307 | 0 | 0 | 0 | 0 | 0 | 0 | 0.7 | 0 | 0 | 0 |
| | 308 | 0 | 0 | 0 | 0 | 0 | 0 | 0 | 0 | 0 | 0 |
| | 309 | 0 | 0 | 0 | 0 | 0 | 0 | 0 | 0 | 0.7 | 0 |
| | 310 | 0 | 0 | 0 | 0 | 0 | 0 | 0 | 0 | 0 | 0 |
| | 311 | 0 | 0 | 0 | 0.7 | 0 | 0 | 0 | 0 | 0 | 0 |
| | 312 | 0 | 0 | 0 | 0 | 0 | 0 | 0 | 0 | 0 | 0 |
| | 313 | 0 | 0 | 0.7 | 0 | 0 | 0 | 0 | 0 | 0 | 0 |
| | 314 | 0 | 0 | 0 | 0 | 0 | 0 | 0 | 0 | 0.7 | 0 |
| | 315 | 0.7 | 0 | 0 | 0 | 0 | 0 | 0 | 0 | 0 | 0 |
| | 316 | 0 | 0 | 0 | 0 | 0 | 0 | 0 | 0 | 0 | 0 |
| | 317 | 0 | 0 | 0 | 0 | 1 | 0 | 0 | 0.7 | 0 | 0 |
| | 318 | 0 | 0 | 0 | 0 | 0 | 1 | 0 | 0 | 0 | 0 |
| | 319 | 0 | 0 | 0 | 0 | 0 | 0 | 0.7 | 0 | 0 | 0 |
| | 320 | 0 | 0 | 0 | 0 | 0 | 0 | 0 | 0 | 1 | 0 |
| | 321 | 0 | 0 | 0 | 0 | 0 | 0 | 0 | 0 | 0 | 0 |
| | 322 | 0 | 0 | 0 | 1 | 0 | 0 | 0 | 0 | 0 | 0 |
| | 323 | 0 | 0 | 0 | 0 | 0 | 0 | 0 | 0 | 0 | 0 |
| | 324 | 0 | 0 | 0 | 0.7 | 0 | 0 | 0 | 0 | 0 | 0 |
| | 325 | 0 | 0 | 0 | 0 | 0 | 0 | 0 | 0 | 0 | 0 |
| | 326 | 0 | 0 | 0 | 0 | 0 | 0 | 0 | 0 | 0 | 0 |
| | 327 | 0 | 0 | 0.7 | 0 | 0 | 0 | 0 | 0 | 0 | 0 |
| | 328 | 0 | 0 | 0 | 0 | 0 | 0 | 0 | 0 | 0 | 0 |
| | 329 | 0 | 0 | 0 | 0 | 0 | 0 | 0 | 0 | 0 | 0 |
| | 330 | 0 | 0 | 0 | 0 | 0 | 0 | 0 | 0 | 0 | 0 |
| | 331 | 0.7 | 0 | 0 | 0 | 0 | 0 | 0 | 0 | 0 | 0 |
| | 332 | 0 | 0 | 0 | 0 | 0 | 0 | 0 | 0 | 0 | 0 |
| | 333 | 0 | 0 | 0 | 0 | 0 | 0 | 0 | 0 | 0 | 0 |
| | 334 | 0 | 0 | 0 | 0 | 0 | 0 | 0 | 0 | 1 | 0 |
| | 335 | 0 | 0 | 0 | 0 | 0 | 0 | 0 | 0 | 0.7 | 0 |
| | 336 | 0.7 | 0 | 0 | 0 | 0 | 0 | 0 | 0 | 0 | 0 |
| | 337 | 0 | 0 | 0 | 0 | 0.7 | 0 | 0 | 0 | 0 | 0 |
| | 338 | 0 | 0 | 0 | 0 | 0 | 0 | 0 | 0 | 0 | 0 |
| | 339 | 0 | 0 | 0 | 0.7 | 0 | 0 | 0 | 0 | 0 | 0 |
| | 340 | 0 | 0 | 0 | 0 | 0 | 0 | 0 | 0 | 0 | 0 |
| | 341 | 0 | 0 | 0 | 0 | 0 | 0 | 0 | 0 | 0 | 0 |
| | 342 | 0 | 0 | 0 | 0 | 0 | 0 | 0 | 0 | 0 | 0 |
| | 343 | 0 | 0 | 0 | 0 | 0 | 0 | 0 | 0 | 0.7 | 0 |
| | 344 | 0 | 0 | 0 | 0 | 0 | 0 | 0 | 0 | 0 | 0 |
| | 345 | 0 | 0 | 0 | 0 | 0 | 0 | 0 | 0 | 0 | 0 |
| | 346 | 0 | 0 | 0 | 0 | 0 | 0 | 0 | 0 | 0 | 0 |
| | 347 | 0.7 | 0 | 0 | 0 | 0 | 0 | 0 | 0 | 0 | 0 |
| | 348 | 0 | 0 | 0 | 0 | 0 | 0 | 0 | 0 | 0 | 0 |
| | 349 | 0 | 0 | 0 | 0 | 0 | 0 | 0 | 0 | 0 | 0 |
| | 350 | 0 | 0 | 0 | 0 | 0 | 0 | 0 | 0 | 0 | 0 |
| | 351 | 0 | 0 | 0 | 0 | 0 | 0 | 0 | 0 | 0 | 0 |
| | 352 | 0 | 0 | 0.7 | 0 | 0 | 0 | 0 | 0 | 0 | 0 |
| | 353 | 0 | 0 | 0 | 0 | 0 | 0 | 0.7 | 0 | 0 | 0 |
| | 354 | 0 | 0 | 0 | 0 | 0 | 0.7 | 0 | 0 | 0 | 0 |
| | 355 | 0 | 0 | 0 | 0 | 0 | 0 | 0 | 0 | 0 | 0 |
| | 356 | 0 | 0 | 0 | 0 | 0 | 0 | 0 | 0 | 0 | 0 |
| | 357 | 0 | 0 | 0 | 0 | 0 | 0 | 0 | 0 | 0 | 0 |
| | 358 | 0 | 0 | 0 | 0 | 0 | 0 | 0.7 | 0 | 0 | 0 |
| | 359 | 0 | 0 | 0 | 0 | 0 | 0 | 0 | 0 | 0 | 0 |
| | 360 | 0 | 0 | 0 | 0 | 0 | 0 | 0 | 0 | 0 | 0 |
| | 361 | 0 | 0 | 0 | 0 | 0 | 0.7 | 0 | 0 | 0 | 0 |
| | 362 | 0 | 0.7 | 0 | 0 | 0 | 0 | 0 | 0 | 0 | 0 |
| | 363 | 0 | 0 | 0 | 0 | 0 | 0 | 0 | 0 | 0 | 0 |
| | 364 | 0 | 0 | 0 | 0 | 1 | 0 | 0 | 0 | 0 | 0 |
| | 365 | 0 | 0 | 0 | 0 | 0 | 0 | 0 | 0 | 0.7 | 0 |
| | 366 | 0 | 0 | 0 | 0 | 0 | 0 | 0 | 0 | 0 | 0 |
| | 367 | 0 | 0 | 0 | 0 | 0 | 0 | 0 | 0 | 0 | 0 |
| | 368 | 0 | 0 | 0 | 0 | 0 | 0 | 0 | 0 | 0 | 0 |
| | 369 | 0 | 0 | 0 | 0 | 0 | 0 | 0 | 0 | 0.7 | 0 |
| | 370 | 0 | 0 | 0 | 0.7 | 0 | 0 | 0 | 0 | 0 | 0 |
| | 371 | 0 | 0.7 | 0 | 0 | 0 | 0 | 0 | 0 | 0 | 0 |
| | 372 | 0 | 0 | 0 | 0 | 0 | 0 | 0 | 0 | 0 | 0 |
| | 373 | 0 | 0 | 0.7 | 0 | 0 | 0 | 0 | 0 | 0 | 0 |
| | 374 | 0 | 0 | 0 | 0 | 0.7 | 0 | 0 | 0 | 0 | 0 |
| | 375 | 0 | 0 | 0 | 1 | 0 | 0 | 0 | 0 | 0 | 0 |
| | 376 | 0 | 0 | 0.7 | 0 | 0 | 0 | 0 | 0 | 0 | 0 |
| | 377 | 0 | 0 | 0 | 0 | 0 | 0 | 0 | 0 | 0 | 0 |
| | 378 | 0 | 0 | 0 | 0 | 0 | 0 | 0 | 0 | 0 | 0 |
| | 379 | 0 | 0 | 0 | 0 | 0 | 0 | 0 | 0 | 0 | 0 |
| | 380 | 0 | 0 | 0 | 0 | 0 | 0 | 0 | 0 | 0 | 0 |
| | 381 | 0 | 0 | 0 | 0.7 | 0 | 0 | 0 | 0 | 0 | 0 |
| | 382 | 0 | 0 | 0 | 0 | 0 | 0 | 0 | 0 | 0 | 0 |
| | 383 | 0 | 0 | 0 | 0 | 0 | 0 | 0 | 0 | 0 | 0 |
| | 384 | 0 | 0 | 0 | 0 | 0 | 0 | 0 | 0 | 0 | 0 |
| | 385 | 0 | 0 | 0 | 0 | 0 | 0 | 0 | 0 | 0 | 0 |
| | 386 | 0 | 0 | 0 | 0 | 0 | 0 | 0 | 0.7 | 0 | 0 |
| | 387 | 0 | 0.7 | 0 | 0 | 0 | 0 | 0 | 0 | 0 | 0 |
| | 388 | 0 | 0 | 0 | 0 | 0.7 | 0 | 0 | 0 | 0 | 0 |
| | 389 | 0 | 0 | 0 | 0 | 0 | 0 | 0 | 0 | 0 | 0 |
| | 390 | 0 | 0 | 0 | 0 | 0 | 0 | 0 | 0 | 0.7 | 0 |
| | 391 | 0 | 0 | 0 | 0 | 0 | 0 | 0 | 0 | 0 | 0 |
| | 392 | 1 | 0 | 0 | 0 | 0 | 0 | 0 | 0 | 0 | 0 |
| | 393 | 0 | 0 | 0 | 0 | 0 | 0 | 0 | 0 | 0.7 | 0 |
| | 394 | 0 | 0 | 0 | 0 | 0 | 0 | 0 | 0 | 0 | 0 |
| | 395 | 0 | 0 | 0 | 0 | 0 | 0 | 0 | 0 | 0.7 | 0 |
| | 396 | 0 | 0 | 0 | 0 | 0 | 0 | 0 | 0.7 | 0 | 0 |
| | 397 | 0 | 0 | 0 | 0 | 0 | 0 | 0 | 0 | 0 | 0 |
| | 398 | 0 | 0 | 0 | 0 | 0 | 0 | 0 | 0 | 0 | 0 |
| | 399 | 0 | 0 | 0 | 0 | 0 | 0 | 0 | 0 | 0 | 0 |
| | 400 | 0 | 0 | 0 | 0 | 0 | 0 | 0 | 0 | 0 | 0 |
| | 401 | 0 | 0 | 0.7 | 0 | 0 | 0 | 1 | 0 | 0 | 0 |
| | 402 | 0 | 0 | 0 | 0 | 0 | 0 | 0 | 0 | 0 | 0 |
| | 403 | 0 | 0 | 0 | 0 | 0 | 0 | 0 | 0 | 0 | 0 |
| | 404 | 0 | 0 | 0 | 0 | 0 | 0 | 0 | 0 | 0 | 0 |
| | 405 | 0 | 0 | 0 | 0 | 0 | 0 | 0 | 0 | 0 | 0 |
| | 406 | 0 | 0 | 0 | 0 | 0 | 0 | 0 | 0 | 0 | 0 |
| | 407 | 0 | 0 | 0 | 0 | 0 | 0 | 0 | 0 | 0 | 0 |
| | 408 | 0 | 0 | 0 | 0 | 0 | 0 | 0 | 0 | 0 | 0 |
| | 409 | 0 | 0 | 0 | 0 | 0 | 0 | 0 | 0 | 0 | 0 |
| | 410 | 0 | 0 | 0 | 0 | 0 | 0 | 0 | 0 | 0.7 | 0 |
| | 411 | 0 | 0 | 0 | 0 | 0 | 0 | 0 | 0 | 0 | 0 |
| | 412 | 0 | 0 | 0 | 0 | 0 | 0 | 0 | 0 | 0 | 0 |
| | 413 | 0 | 0 | 0 | 0 | 0 | 0 | 0 | 0 | 0.7 | 0 |
| | 414 | 0 | 0.7 | 0 | 0 | 0 | 0 | 0 | 0 | 0 | 0 |
| | 415 | 0 | 0 | 0 | 0 | 0 | 0 | 0 | 0 | 0.7 | 0 |
| | 416 | 0 | 0 | 0 | 0 | 0 | 0 | 0 | 0 | 0 | 0 |
| | 417 | 0 | 0 | 0 | 0.7 | 0 | 0 | 0 | 0 | 0 | 0 |
| | 418 | 0 | 0 | 0 | 0 | 0 | 0 | 0 | 0 | 0 | 0 |
| | 419 | 0 | 0 | 0 | 0 | 0 | 0 | 0 | 0 | 0 | 0 |
| | 420 | 0 | 0 | 0 | 0 | 0 | 0 | 0 | 0 | 0 | 0 |
| | 421 | 0 | 0 | 0 | 0 | 0 | 0 | 0 | 1 | 0.7 | 0 |
| | 422 | 0 | 0.7 | 0 | 0 | 0 | 0 | 0 | 0 | 0 | 0 |
| | 423 | 0 | 0 | 0 | 0 | 0 | 0 | 0 | 0 | 0 | 0 |
| | 424 | 0 | 0 | 0 | 0 | 0 | 0 | 0 | 0 | 0 | 0 |
| | 425 | 0 | 0 | 0 | 0 | 0 | 0 | 0 | 0 | 0 | 0 |
| | 426 | 0 | 0 | 0 | 0 | 0 | 0 | 0 | 0 | 0 | 0 |
| | 427 | 0 | 0 | 0 | 0 | 0 | 0 | 0 | 0 | 0 | 0 |
| | 428 | 0.7 | 0 | 0 | 0 | 0 | 0 | 0 | 0 | 1 | 0 |
| | 429 | 0 | 0 | 0 | 0 | 0 | 0 | 0 | 0 | 0 | 0 |
| | 430 | 0.7 | 0 | 0 | 0 | 0 | 0 | 0 | 0 | 0 | 0 |
| | 431 | 0 | 0 | 0 | 0 | 0 | 0 | 0 | 0 | 0 | 0 |
| | 432 | 0.7 | 0 | 0 | 0 | 0 | 0 | 0 | 0 | 0 | 0 |
| | 433 | 0 | 0 | 0 | 0 | 0 | 0 | 0 | 0 | 0 | 0 |
| | 434 | 0 | 0 | 0 | 0 | 0 | 0 | 0 | 0 | 0 | 0 |
| | 435 | 0 | 0 | 0 | 0 | 0 | 0 | 0 | 0 | 1 | 0 |
| | 436 | 0 | 0 | 0 | 0 | 0.7 | 0 | 0 | 0 | 0 | 0 |
| | 437 | 0 | 0 | 0 | 0 | 0 | 0 | 0 | 0 | 0 | 0 |
| | 438 | 0 | 1 | 0 | 0 | 0 | 0 | 0 | 0 | 0 | 0 |
| | 439 | 1 | 0 | 0 | 0.7 | 0 | 0 | 0 | 0 | 0 | 0 |
| | 440 | 0 | 0 | 0 | 0 | 0 | 0 | 0 | 0 | 0 | 0 |
| | 441 | 0 | 0 | 0 | 0 | 0 | 0 | 0 | 0 | 0 | 0 |
| | 442 | 0 | 0 | 0 | 0 | 0.7 | 0 | 0 | 0 | 0 | 0 |
| | 443 | 0 | 0 | 0 | 0 | 0.7 | 0 | 0 | 0 | 0 | 0 |
| | 444 | 0 | 0 | 0 | 0 | 0 | 0 | 0 | 0.7 | 0 | 0 |
| | 445 | 0 | 0 | 0 | 0 | 0 | 0 | 0 | 0 | 0 | 0 |
| | 446 | 0 | 0 | 0 | 0 | 0.7 | 0 | 0 | 0 | 0 | 0 |
| | 447 | 0 | 0 | 0 | 0 | 0 | 0 | 0 | 0 | 0 | 0 |
| | 448 | 0 | 0 | 0 | 0 | 0 | 0 | 0 | 0 | 0 | 0 |
| | 449 | 0 | 0 | 0 | 0 | 1 | 0 | 0 | 0 | 0 | 0 |
| | 450 | 0 | 0 | 0 | 0 | 0 | 0 | 0 | 0 | 0 | 0 |
| | 451 | 0 | 0 | 0 | 0 | 0 | 0 | 0 | 0 | 0.7 | 0 |
| | 452 | 0 | 0 | 0 | 0 | 0.7 | 0 | 0 | 0 | 0 | 0 |
| | 453 | 0 | 0 | 0 | 0 | 0 | 0 | 0 | 0.7 | 0 | 0 |
| | 454 | 0 | 0 | 0 | 0 | 0 | 0 | 0 | 0 | 0 | 0 |
| | 455 | 0 | 0 | 0 | 0 | 0.7 | 0 | 0 | 0 | 0 | 0 |
| | 456 | 0 | 0 | 0 | 0 | 0.7 | 0 | 0 | 0 | 0 | 0 |
| | 457 | 0 | 0 | 0 | 0 | 0 | 0 | 0 | 0 | 0 | 0 |

TABLE 9
Position Specific Scoring Matrix (PSSM) of Successful B13GT Designs

| Seq | Amino Acid | | | | | | | | | | | | | | | | | | | | |
|---|---|---|---|---|---|---|---|---|---|---|---|---|---|---|---|---|---|---|---|---|---|
| Pos | A | R | N | D | C | Q | E | G | H | I | L | K | M | F | P | S | T | W | Y | V | — |
| 1 | 0 | 0 | 0 | 0 | 0 | 0 | 0 | 0 | 0 | 0 | 0 | 0 | .11 | 0 | 0 | 0 | 0 | 0 | 0 | 0 | 0 |
| 2 | 0 | .11 | .11 | 0 | 0 | .11 | .11 | .11 | .11 | 0 | .11 | .11 | .11 | .11 | .11 | 0 | 0 | 0 | 0 | .11 | 0 |
| 3 | 0 | 0 | .11 | 0 | 0 | 0 | 0 | 0 | 0 | 0 | .11 | 0 | .11 | .11 | 0 | .11 | 0 | 0 | 0 | 0 | 0 |
| 4 | 0 | 0 | .11 | 0 | 0 | 0 | 0 | 0 | 0 | 0 | 0 | .11 | 0 | 0 | .11 | .11 | .11 | 0 | 0 | 0 | 0 |
| 5 | .11 | 0 | .11 | 0 | 0 | .11 | 0 | 0 | .11 | 0 | .11 | 0 | .11 | 0 | 0 | .11 | .11 | 0 | 0 | 0 | 0 |
| 6 | 0 | 0 | 0 | 0 | 0 | 0 | .11 | 0 | 0 | 0 | 0 | 0 | 0 | 0 | .11 | 0 | 1 | 0 | 0 | 0 | 0 |
| 7 | .11 | 0 | 0 | 0 | 0 | .11 | 0 | 0 | 0 | 0 | .11 | 0 | 0 | 0 | .11 | 0 | .11 | 0 | 0 | 0 | 0 |
| 8 | 0 | 0 | 0 | .11 | 0 | 0 | 0 | 0 | 0 | 0 | .11 | 0 | 0 | 0 | 0 | 0 | .11 | 0 | 0 | 1 | 0 |
| 9 | .11 | 1 | 0 | 0 | 0 | 0 | 0 | .11 | 0 | 0 | .11 | .11 | .11 | 0 | 0 | .11 | 0 | 0 | 0 | .11 | 0 |
| 10 | 0 | .11 | 0 | 0 | 0 | 0 | 0 | 0 | 0 | 0 | 0 | .11 | 0 | 0 | 0 | 0 | 0 | 0 | 0 | 0 | 0 |
| 11 | 0 | .11 | 0 | 0 | 0 | 0 | 0 | 0 | 0 | 0 | 0 | 0 | 0 | 0 | 0 | 0 | 1 | 0 | 0 | 0 | 0 |
| 12 | 0 | .11 | 0 | 0 | 0 | .11 | 0 | 0 | 0 | 0 | 0 | 0 | 0 | 0 | 0 | .11 | 0 | 0 | 0 | 0 | 0 |
| 13 | 0 | .11 | 0 | 0 | 0 | 0 | 0 | 0 | 0 | 0 | 0 | 0 | 0 | 0 | 0 | 0 | 1 | 0 | 0 | 0 | 0 |
| 14 | 0 | 0 | 0 | 0 | 0 | 0 | 0 | 0 | 0 | .11 | .11 | 0 | 0 | 0 | 0 | 0 | 0 | 0 | 0 | .11 | 0 |
| 15 | 0 | 0 | 0 | 0 | 0 | 0 | 0 | 0 | 0 | .11 | 0 | 0 | 0 | 0 | 0 | 0 | 0 | 0 | 0 | 1 | 0 |
| 16 | 0 | 0 | 0 | 0 | 0 | 0 | 0 | 0 | 0 | 0 | .11 | 0 | .11 | 0 | 0 | 0 | 0 | 0 | 0 | 0 | 0 |
| 17 | 0 | 0 | 0 | 0 | 0 | 0 | 0 | 0 | 0 | 0 | 0 | 0 | 0 | .11 | 0 | 0 | 0 | 0 | 0 | 0 | 0 |
| 18 | 0 | 0 | 0 | 0 | 0 | 0 | 0 | 0 | 0 | 0 | 0 | 0 | 0 | 0 | .11 | 0 | 0 | 0 | 0 | 0 | 0 |
| 19 | 0 | 0 | 0 | 0 | 0 | .11 | 0 | 0 | 0 | 0 | .11 | 0 | 0 | 0 | 0 | 0 | 0 | 0 | 0 | .11 | 0 |
| 20 | 0 | 0 | 0 | 0 | 0 | 0 | 0 | 0 | 0 | 0 | 0 | 0 | 0 | 0 | .11 | 0 | 0 | 0 | 0 | 0 | 0 |
| 21 | 0 | 0 | 0 | 0 | 0 | 0 | 0 | 0 | 0 | 0 | 0 | 0 | 0 | .11 | 0 | 0 | 0 | 0 | 0 | 0 | 0 |
| 22 | .11 | 0 | 0 | 0 | 0 | .11 | 0 | 0 | .11 | 0 | .11 | 0 | 0 | 0 | .11 | 0 | 0 | 0 | 0 | 0 | 0 |
| 23 | 0 | 0 | 0 | 0 | 0 | 0 | 0 | .11 | 0 | 0 | 0 | 0 | 0 | 0 | 0 | 0 | 0 | 0 | 0 | 0 | 0 |
| 24 | 0 | 0 | 0 | 0 | 0 | 0 | 0 | 0 | .11 | 0 | 0 | 0 | 0 | 0 | 0 | 0 | 0 | 0 | 0 | 0 | 0 |
| 25 | .11 | 0 | 0 | 0 | 0 | .11 | 0 | 0 | .11 | .11 | .11 | 0 | .11 | 0 | 0 | .11 | 0 | 1 | 0 | .11 | 0 |
| 26 | 0 | 0 | .11 | 0 | 0 | 0 | 0 | 0 | 0 | 0 | 0 | 0 | 0 | 0 | 0 | 0 | 0 | 0 | 0 | 0 | 0 |
| 27 | 0 | 0 | 0 | 0 | 0 | 0 | 0 | 0 | 0 | 0 | 0 | 0 | 0 | 0 | .11 | 0 | 0 | 0 | 0 | 0 | 0 |
| 28 | 0 | 0 | 0 | 0 | 0 | 0 | 0 | 0 | 0 | .11 | 0 | 0 | .11 | 0 | 0 | 0 | 0 | 0 | 0 | 0 | 0 |
| 29 | 0 | 0 | 0 | 0 | 0 | 0 | 0 | 0 | 0 | 0 | .11 | 0 | .11 | 0 | 0 | 0 | 0 | 0 | 0 | 0 | 0 |
| 30 | 0 | 0 | 0 | 0 | 0 | .11 | 0 | 0 | 0 | 0 | 0 | 0 | 0 | 0 | 0 | 0 | 0 | 0 | 0 | 0 | 0 |
| 31 | 0 | 0 | 0 | 0 | 0 | 0 | 0 | 0 | 0 | 0 | .11 | 0 | 0 | 0 | 0 | 0 | 0 | 0 | 0 | 0 | 0 |
| 32 | .11 | 0 | 0 | 0 | 0 | 0 | 0 | 0 | 0 | 0 | 0 | 0 | 0 | 0 | 0 | 0 | 0 | 0 | 0 | 0 | 0 |
| 33 | 0 | 0 | .11 | 0 | 0 | 0 | 0 | 0 | 0 | 0 | 0 | 0 | 0 | 0 | 0 | 0 | 0 | 0 | 0 | 0 | 0 |
| 34 | 0 | 0 | 0 | 0 | 0 | 0 | 0 | 0 | 0 | .11 | 0 | 0 | 0 | 0 | 0 | 0 | 0 | 0 | 0 | .11 | 0 |
| 35 | 0 | 0 | 0 | 0 | 0 | 0 | 0 | 0 | 0 | 0 | .11 | 0 | 0 | 0 | 0 | 0 | 0 | 0 | 0 | 0 | 0 |
| 36 | 0 | 0 | 0 | 0 | 0 | 0 | 0 | 0 | 0 | 0 | 0 | 0 | 0 | 0 | 0 | 0 | 0 | 0 | .11 | 0 | 0 |
| 37 | 0 | .11 | 0 | 0 | 0 | 0 | 0 | 0 | 0 | 0 | 0 | 0 | 0 | 0 | 0 | .11 | 0 | 0 | 0 | 0 | 0 |
| 38 | 0 | .11 | 0 | 0 | 0 | .11 | 0 | 0 | 0 | 0 | 0 | .11 | 0 | 0 | 0 | 0 | 0 | 0 | 0 | 0 | 0 |
| 39 | 0 | 0 | 0 | 0 | 0 | 0 | 0 | .11 | 0 | 0 | 0 | 0 | 0 | 0 | 0 | 0 | 0 | 0 | 0 | 0 | 0 |
| 40 | 0 | 0 | 0 | 0 | 0 | 0 | 0 | 0 | 0 | 0 | 0 | 0 | 0 | .11 | 0 | 0 | 0 | 0 | 0 | 0 | 0 |
| 41 | .11 | 1 | 0 | 0 | 0 | 0 | .11 | 0 | 0 | 0 | 0 | 0 | 0 | 0 | 0 | .11 | 0 | 0 | 0 | 0 | 0 |
| 42 | 0 | 0 | 0 | 0 | 0 | 0 | 0 | 0 | 0 | .11 | 0 | 0 | 0 | 0 | 0 | 0 | 0 | 0 | 0 | 0 | 0 |
| 43 | 0 | 0 | 0 | 0 | 0 | 0 | 0 | 0 | 0 | 1 | 0 | 0 | 0 | 0 | 0 | 0 | .11 | 0 | 0 | 1 | 0 |
| 44 | 0 | 0 | 0 | 0 | 0 | 0 | 0 | 0 | 0 | .11 | 0 | 0 | 0 | 0 | 0 | 0 | 0 | 0 | 0 | .11 | 0 |
| 45 | 0 | 0 | 0 | 0 | 0 | 0 | 0 | 0 | 0 | 0 | .11 | 0 | 0 | .11 | 0 | 0 | 0 | 0 | 0 | 0 | 0 |
| 46 | 0 | 0 | 0 | 0 | 0 | 0 | 0 | 0 | .11 | 0 | 0 | 0 | 0 | 0 | 0 | .11 | 0 | 0 | 1 | 0 | 0 |
| 47 | 0 | 0 | 0 | 0 | 0 | 0 | 0 | 0 | 0 | 0 | 0 | 0 | 0 | 0 | 0 | 0 | .11 | 0 | 0 | 0 | 0 |
| 48 | 0 | 1 | .11 | 0 | 0 | 1 | 1 | 0 | 0 | 0 | 0 | 0 | 0 | 0 | 0 | 1 | 0 | 0 | 0 | 0 | 0 |
| 49 | .11 | 0 | 0 | 0 | 0 | 0 | 0 | 0 | 0 | 0 | 0 | 0 | 0 | .11 | 0 | 0 | 0 | 0 | 0 | 0 | 0 |
| 50 | 0 | 0 | .11 | 0 | 0 | 0 | 0 | 0 | 0 | 0 | 0 | 0 | 0 | 0 | 0 | 0 | 0 | 0 | 0 | 0 | 0 |
| 51 | .11 | 0 | 0 | 0 | 0 | 0 | 0 | 0 | 0 | 0 | 0 | .11 | 0 | 0 | 0 | 0 | 0 | 0 | 0 | 0 | 0 |
| 52 | 0 | 0 | 0 | 0 | 0 | 0 | 0 | 0 | 0 | 0 | 0 | 0 | 0 | 0 | .11 | 0 | 0 | 0 | 0 | 0 | 0 |
| 53 | 0 | .11 | 0 | 0 | .11 | .11 | .11 | 0 | 0 | 0 | .11 | .11 | 0 | 0 | 0 | 0 | 0 | 0 | 0 | .11 | 0 |
| 54 | .11 | 0 | 0 | 0 | 0 | 0 | 0 | 0 | 0 | 0 | 0 | 0 | 0 | 0 | .11 | 0 | .11 | 0 | 0 | .11 | 0 |
| 55 | 0 | 0 | 0 | 0 | 0 | 0 | 0 | .11 | 0 | 0 | 0 | 0 | 0 | 0 | 0 | .11 | .11 | .11 | 0 | .11 | 0 |
| 56 | .11 | 0 | .11 | .11 | 0 | 0 | 0 | 0 | .11 | 0 | .11 | 0 | 0 | 0 | 0 | 0 | 0 | 0 | 0 | 0 | 0 |
| 57 | 0 | 0 | 0 | 0 | 0 | 0 | 0 | 0 | 0 | 0 | 0 | 0 | 0 | 0 | 0 | 0 | 0 | 0 | .11 | 0 | 0 |
| 58 | 0 | 0 | 0 | 0 | 0 | 0 | 0 | 0 | 0 | 0 | 0 | 0 | 0 | 0 | .11 | 0 | 0 | 0 | 0 | 0 | 0 |
| 59 | 0 | 0 | 0 | 0 | 0 | .11 | 0 | 0 | .11 | 0 | 0 | 0 | 0 | 0 | 0 | 0 | 0 | 0 | 0 | 0 | 0 |
| 60 | 0 | 0 | 0 | 0 | 0 | 0 | 0 | 0 | 0 | 0 | 0 | 0 | 0 | .11 | 0 | 0 | 0 | 0 | 0 | 0 | 0 |
| 61 | 0 | 0 | 0 | .11 | 0 | .11 | .11 | 0 | 0 | 0 | 0 | 0 | 0 | 0 | 0 | .11 | .11 | 0 | 0 | 0 | 0 |
| 62 | 0 | 0 | 0 | 0 | 0 | 0 | 0 | 0 | 0 | 0 | 0 | 0 | 0 | .11 | 0 | 0 | 0 | 0 | 0 | 0 | 0 |
| 63 | 0 | .11 | 0 | 0 | 0 | 0 | 0 | 0 | 0 | 0 | 0 | 0 | 0 | 0 | 0 | 0 | 0 | 0 | 0 | 1 | 0 |
| 64 | 0 | 0 | 0 | 0 | 0 | 1 | 1 | 0 | 0 | 0 | .11 | 0 | .11 | .11 | .11 | .11 | 1 | 0 | 0 | 0 | 0 |
| 65 | 0 | 0 | 0 | 0 | 0 | 0 | 0 | 0 | 0 | .11 | 0 | 0 | 0 | 0 | 0 | 0 | 0 | 0 | 0 | .11 | 0 |
| 66 | 0 | 0 | 0 | 0 | 0 | 0 | 0 | 0 | 0 | 0 | .11 | 0 | 0 | 0 | 0 | 0 | 0 | 0 | 0 | 0 | 0 |
| 67 | 0 | 0 | 0 | .11 | 0 | 0 | 0 | 0 | 0 | 0 | 0 | 0 | 0 | 0 | 0 | 0 | 0 | 0 | 0 | 0 | 0 |
| 68 | 0 | 0 | .11 | 0 | 0 | 0 | 0 | 0 | 0 | 0 | 0 | .11 | 0 | 0 | 0 | 0 | 0 | 0 | 0 | 0 | 0 |
| 69 | .11 | 0 | .11 | .11 | 0 | .11 | 0 | 0 | 0 | 0 | 0 | 0 | .11 | 0 | 0 | .11 | .11 | 0 | 0 | 0 | 0 |
| 70 | 0 | 0 | 0 | 0 | 0 | 0 | 0 | 0 | 0 | 0 | 0 | .11 | 0 | 0 | .11 | .11 | 0 | 0 | 0 | 0 | 0 |
| 71 | 0 | .11 | 0 | 0 | 0 | .11 | 0 | .11 | 0 | 0 | .11 | 0 | 0 | 0 | 0 | 0 | .11 | 0 | 0 | .11 | 0 |
| 72 | 0 | 0 | 0 | .11 | 0 | 0 | .11 | 0 | 0 | 0 | 0 | 0 | 0 | 0 | .11 | .11 | .11 | 0 | 0 | 0 | 0 |
| 73 | 0 | 0 | 0 | 0 | 0 | 0 | .11 | 0 | 0 | 0 | 0 | 0 | 0 | 0 | .11 | .11 | .11 | 0 | 0 | 0 | 0 |
| 74 | 0 | .11 | 0 | 0 | 0 | 0 | 0 | 0 | .11 | 0 | 0 | 0 | 0 | 0 | 0 | 0 | 0 | .11 | 0 | 0 | 0 |
| 75 | 0 | 0 | 0 | 0 | 0 | 0 | 0 | .11 | 0 | .11 | .11 | 0 | .11 | 0 | 0 | 0 | 0 | 0 | 0 | 0 | 0 |
| 76 | 0 | .11 | 0 | 0 | 0 | 0 | 0 | 0 | 0 | 0 | 0 | 0 | 0 | 0 | 0 | .11 | .11 | 0 | 0 | 0 | 0 |
| 77 | 0 | 0 | .11 | 0 | 0 | 0 | 0 | 0 | 0 | 0 | 0 | 0 | 0 | 0 | 0 | 0 | 0 | 0 | 0 | 0 | 0 |
| 78 | 0 | .11 | 0 | 0 | 0 | .11 | .11 | .11 | 0 | .11 | .11 | .11 | .11 | .11 | .11 | 0 | 0 | 0 | 0 | .11 | 0 |
| 79 | 0 | 0 | 0 | 0 | 0 | 0 | 0 | .11 | 0 | 0 | 0 | 0 | 0 | 0 | .11 | .11 | 0 | 0 | 0 | .11 | 0 |
| 80 | .11 | 0 | 0 | .11 | 0 | .11 | 0 | 0 | 0 | 0 | .11 | 0 | 0 | 0 | .11 | 0 | .11 | 0 | 0 | 0 | 0 |
| 81 | .11 | 0 | 0 | 0 | .11 | 0 | 0 | 0 | .11 | 0 | .11 | 0 | .11 | 0 | 0 | .11 | .11 | 0 | 0 | .11 | 0 |
| 82 | 0 | 0 | 0 | 0 | 0 | 0 | 0 | .11 | 0 | 0 | 0 | 0 | 0 | 0 | 0 | 0 | 0 | 0 | 0 | 0 | 0 |
| 83 | .11 | 0 | .11 | .11 | 0 | 0 | 0 | 0 | 0 | 0 | 0 | .11 | 0 | 0 | .11 | .11 | .11 | 0 | 0 | 0 | 0 |
| 84 | .11 | 0 | .11 | .11 | 0 | 0 | 0 | .11 | .11 | 0 | .11 | 0 | 0 | 0 | 0 | 0 | 0 | 0 | 0 | .11 | 0 |
| 85 | .11 | 0 | 0 | 0 | 0 | 0 | .11 | 0 | 0 | 0 | .11 | 0 | 0 | 0 | 0 | 0 | .11 | 0 | 0 | .11 | 0 |
| 86 | 0 | 0 | 0 | 0 | 0 | 0 | 0 | .11 | 0 | 0 | 0 | 0 | 0 | 0 | 0 | 0 | 0 | 0 | 0 | 0 | 0 |
| 87 | .11 | .11 | 0 | 0 | 0 | .11 | .11 | 0 | .11 | 0 | .11 | .11 | .11 | 0 | 0 | 0 | 0 | .11 | 0 | 0 | 0 |
| 88 | .11 | .11 | 0 | 0 | .11 | 0 | 0 | 0 | .11 | .11 | .11 | .11 | .11 | 0 | 0 | 0 | .11 | 0 | 0 | .11 | 0 |
| 89 | 0 | 0 | 0 | 0 | 0 | 0 | 0 | 0 | 0 | .11 | 0 | 0 | 0 | 0 | 0 | 0 | 0 | 0 | 0 | 0 | 0 |
| 90 | 0 | 0 | 0 | 0 | 0 | .11 | 0 | 0 | 0 | 0 | 0 | 0 | 0 | 0 | .11 | .11 | .11 | 0 | 0 | 0 | 0 |
| 91 | 0 | .11 | .11 | 0 | 0 | .11 | 0 | 0 | 0 | .11 | .11 | 0 | 0 | 0 | 0 | 0 | .11 | 0 | 0 | .11 | 0 |
| 92 | 0 | 0 | 0 | 0 | 0 | 0 | 0 | 0 | 0 | .11 | .11 | 0 | 0 | 0 | 0 | 0 | 0 | 0 | 0 | 0 | 0 |
| 93 | 0 | 0 | .11 | 0 | 0 | 0 | 0 | 0 | 0 | 0 | 0 | 0 | 0 | 0 | 0 | 0 | 0 | 0 | 0 | 0 | 0 |
| 94 | 0 | .11 | 0 | 0 | .11 | 0 | .11 | 0 | 0 | 0 | 0 | .11 | 0 | 0 | 0 | 0 | 0 | 0 | 0 | 0 | 0 |
| 95 | 0 | 0 | .11 | 0 | 0 | 0 | 0 | 0 | .11 | 0 | .11 | 0 | 0 | 0 | 0 | 0 | 0 | .11 | 0 | 0 | 0 |
| 96 | 0 | 0 | 0 | 0 | .11 | 0 | 0 | .11 | 0 | 0 | 0 | .11 | 0 | 0 | 0 | 0 | 0 | 0 | 0 | 0 | 0 |
| 97 | .11 | 0 | 0 | 0 | 0 | 0 | 0 | 0 | 0 | 0 | 0 | 1 | 0 | 0 | 0 | .11 | 0 | 0 | 0 | .11 | 0 |
| 98 | 1 | 0 | 0 | .11 | 0 | 0 | .11 | 0 | 0 | 0 | 0 | 0 | 0 | 0 | .11 | .11 | .11 | 0 | 0 | 0 | 0 |
| 99 | 0 | 1 | 0 | 0 | 0 | 1 | .11 | 0 | 0 | 0 | .11 | 0 | 0 | 0 | .11 | .11 | 0 | 0 | 0 | .11 | 0 |
| 100 | 0 | 0 | 0 | 0 | 0 | 0 | 0 | 0 | 0 | 0 | .11 | 0 | 0 | .11 | 0 | 0 | 0 | 0 | 0 | 0 | 0 |
| 101 | 0 | .11 | 0 | 0 | 0 | 0 | 1 | 0 | 0 | 0 | 0 | 1 | 0 | 0 | 0 | 0 | 0 | 0 | 0 | 0 | 0 |
| 102 | 1 | .11 | 0 | 0 | 0 | 1 | 0 | 0 | 0 | 0 | 0 | .11 | 0 | 0 | 0 | 0 | 0 | 0 | 0 | 0 | 0 |
| 103 | 1 | 0 | 0 | 0 | 0 | 0 | .11 | 0 | 0 | 1 | 1 | 1 | 1 | 0 | 0 | 0 | 1 | 0 | 0 | 0 | 0 |
| 104 | 0 | 0 | 0 | 0 | 0 | 0 | 0 | 0 | 0 | 0 | .11 | 0 | 0 | 0 | 0 | 0 | 0 | 0 | 0 | 0 | 0 |
| 105 | .11 | 0 | 0 | 0 | 0 | 0 | .11 | 0 | 0 | 0 | 1 | 1 | 0 | 0 | 0 | 0 | 0 | 0 | 0 | 0 | 0 |
| 106 | 0 | 0 | 0 | .11 | 0 | 1 | 1 | .11 | 0 | .11 | .11 | 1 | 0 | 0 | 0 | .11 | .11 | 0 | .11 | 0 | 0 |
| 107 | .11 | 0 | 0 | 0 | .11 | 0 | 1 | 0 | 0 | 1 | .11 | 0 | 0 | 0 | 0 | 0 | .11 | 0 | 1 | .11 | 0 |
| 108 | 0 | 0 | 0 | 0 | .11 | 0 | 0 | 0 | .11 | 1 | 0 | 0 | .11 | 0 | 0 | 0 | 0 | 0 | 0 | 0 | 0 |
| 109 | 1 | .11 | 1 | 0 | 0 | 1 | 1 | 0 | 0 | 0 | .11 | 0 | 0 | 0 | 0 | .11 | 0 | .11 | 0 | 0 | 0 |
| 110 | .11 | 0 | 0 | .11 | 0 | 0 | 0 | .11 | 0 | 0 | 0 | 0 | 0 | 0 | 0 | .11 | 0 | 0 | 0 | 0 | 0 |
| 111 | 0 | 0 | 0 | 0 | .11 | 0 | 0 | .11 | 0 | 0 | 0 | 0 | 0 | 0 | 0 | .11 | .11 | 0 | 0 | 0 | 0 |
| 112 | .11 | 0 | .11 | 0 | 0 | 0 | .11 | 0 | 0 | 0 | 0 | 0 | 0 | 0 | .11 | .11 | 0 | 0 | 0 | 0 | 0 |
| 113 | 0 | 0 | 0 | .11 | 0 | 0 | .11 | .11 | 0 | 0 | 0 | 0 | 0 | 0 | .11 | .11 | 0 | 0 | 0 | 0 | 0 |
| 114 | 0 | 0 | 0 | .11 | 0 | 0 | 0 | 0 | 0 | 0 | 0 | 0 | 0 | 0 | .11 | 0 | 0 | 0 | 0 | 0 | 0 |
| 115 | 0 | .11 | 0 | 0 | 0 | 0 | .11 | 0 | 0 | 0 | 0 | 0 | 0 | 0 | 0 | 0 | 0 | 0 | 0 | 0 | 0 |
| 116 | 0 | .11 | 0 | .11 | 0 | 0 | .11 | 0 | 0 | 0 | 0 | 0 | 0 | 0 | 0 | .11 | 1 | 0 | 0 | 0 | 0 |
| 117 | 0 | 0 | 0 | 0 | 0 | 0 | 0 | 0 | 0 | 1 | 1 | 0 | 0 | 0 | 0 | 0 | 0 | 0 | 0 | .11 | 0 |
| 118 | .11 | 0 | 0 | 0 | .11 | 0 | 0 | 0 | 0 | 0 | 0 | 0 | 0 | 0 | 0 | .11 | .11 | 0 | 0 | .11 | 0 |
| 119 | 0 | 0 | 0 | 0 | .11 | 0 | 0 | 0 | 0 | 0 | 0 | 0 | 0 | 0 | 0 | 0 | 0 | 0 | 0 | 0 | 0 |
| 120 | 0 | 0 | 0 | 0 | 0 | 0 | 0 | 0 | 0 | 0 | .11 | 0 | 0 | 0 | 0 | 0 | 0 | 0 | 0 | .11 | 0 |
| 121 | 0 | 0 | 0 | 0 | 0 | 0 | 0 | 0 | 0 | .11 | 0 | 0 | 0 | 0 | 0 | 0 | 0 | 0 | 0 | 0 | 0 |
| 122 | 0 | 0 | 0 | 0 | 0 | 0 | 0 | 0 | 0 | 0 | 0 | 0 | 0 | 0 | 0 | 0 | .11 | 0 | 0 | 0 | 0 |
| 123 | 0 | 0 | 0 | .11 | 0 | 0 | 0 | 0 | 0 | 0 | 0 | 0 | 0 | 0 | 0 | 0 | 0 | 0 | 0 | 0 | 0 |
| 124 | .11 | 0 | 0 | 0 | 0 | 0 | 0 | 0 | 0 | 0 | 0 | 0 | 0 | 0 | .11 | 0 | 0 | 0 | 0 | 0 | 0 |
| 125 | 0 | 0 | 0 | 0 | 0 | 0 | 0 | 0 | 0 | 0 | .11 | 0 | .11 | 0 | 0 | 0 | 0 | 0 | 0 | 0 | 0 |
| 126 | 0 | 0 | 0 | 0 | 0 | 0 | 0 | 0 | 0 | 0 | 0 | 0 | 0 | 0 | 0 | 0 | 0 | .11 | 0 | 0 | 0 |
| 127 | 0 | 0 | 0 | 0 | 0 | 0 | 0 | 0 | 0 | 0 | 0 | 0 | 0 | 0 | 0 | 0 | 0 | 0 | .11 | 0 | 0 |
| 128 | 0 | 0 | 0 | 0 | 0 | 0 | 0 | 0 | 0 | 0 | 0 | 0 | 0 | .11 | 0 | 0 | 0 | 0 | 0 | 0 | 0 |
| 129 | .11 | 0 | 0 | 0 | 0 | 0 | 0 | 0 | 0 | 0 | 0 | 0 | 0 | 0 | .11 | 0 | 1 | 0 | 0 | 0 | 0 |
| 130 | 0 | 0 | 0 | 0 | 0 | .11 | 1 | 1 | 0 | 0 | 0 | 0 | 0 | 0 | 0 | 0 | 0 | 0 | 0 | 0 | 0 |
| 131 | .11 | 0 | 0 | 1 | 0 | 0 | 1 | 0 | 0 | 0 | 0 | 0 | 0 | 0 | .11 | .11 | 0 | 0 | 0 | .11 | 0 |
| 132 | 0 | 0 | 0 | 0 | 0 | 0 | 0 | 0 | 0 | 0 | 0 | 0 | 0 | 0 | 0 | 0 | 0 | 0 | 0 | .11 | 0 |
| 133 | .11 | 0 | 0 | 0 | 0 | 0 | 0 | 0 | 0 | 0 | 0 | 0 | 0 | 0 | 0 | 0 | 0 | 0 | 0 | 0 | 0 |
| 134 | 0 | 0 | 0 | .11 | 0 | 0 | .11 | .11 | 0 | 0 | 0 | 1 | 0 | 0 | 0 | .11 | 0 | 0 | 0 | 0 | 0 |
| 135 | .11 | 0 | 0 | 0 | 0 | 0 | 1 | 0 | 0 | 0 | 0 | 1 | 0 | 0 | 0 | .11 | 0 | 0 | 0 | 0 | 0 |
| 136 | 0 | 0 | 0 | 0 | 0 | 0 | 0 | 0 | 0 | 0 | .11 | 0 | 0 | 0 | 0 | 0 | 0 | 0 | 0 | 0 | 0 |
| 137 | 0 | 0 | .11 | 0 | 0 | 0 | 0 | .11 | 0 | 0 | 0 | .11 | 0 | 0 | 0 | 0 | 0 | 0 | 0 | 0 | 0 |
| 138 | 0 | 0 | 0 | 0 | 0 | 0 | 0 | 0 | 0 | .11 | .11 | 0 | 0 | 0 | 0 | 0 | 0 | 0 | 0 | .11 | 0 |
| 139 | .11 | .11 | .11 | 0 | 0 | 0 | 0 | 0 | 0 | 0 | 0 | .11 | 0 | 0 | .11 | .11 | 0 | 0 | 0 | 0 | 0 |
| 140 | 0 | .11 | 0 | 0 | 0 | 0 | 0 | 0 | 0 | 0 | 1 | 0 | 0 | 0 | 0 | 0 | 0 | 0 | 0 | 0 | 0 |
| 141 | 0 | 0 | 0 | 0 | 0 | 0 | 0 | 0 | 0 | 0 | .11 | 0 | .11 | 0 | 0 | 0 | 0 | 0 | 0 | 0 | 0 |
| 142 | 0 | 0 | 0 | 0 | 0 | 0 | 0 | 0 | 0 | 0 | 0 | 0 | 0 | 0 | 0 | 0 | 0 | 0 | 0 | .11 | 0 |
| 143 | 0 | 0 | 0 | 0 | 0 | 0 | 0 | 0 | 0 | 0 | .11 | 0 | 0 | 0 | 0 | 0 | 0 | 0 | 0 | 0 | 0 |
| 144 | 0 | 0 | 0 | 0 | 0 | .11 | 0 | 0 | 0 | 0 | 0 | 0 | .11 | 0 | 0 | 0 | 0 | 0 | 0 | 0 | 0 |
| 145 | 0 | 0 | 0 | 0 | 0 | 0 | 0 | 0 | 0 | 0 | 0 | 0 | 0 | 0 | 0 | 0 | .11 | 0 | 0 | 0 | 0 |
| 146 | 0 | 0 | 0 | 0 | 0 | 0 | 0 | .11 | 0 | 0 | 0 | 0 | 0 | 0 | 0 | .11 | 0 | 0 | 0 | 0 | 0 |
| 147 | 0 | 0 | 0 | 0 | 0 | 0 | 0 | 0 | 0 | 0 | 0 | 0 | 0 | 0 | 0 | .11 | 0 | 0 | 0 | 0 | 0 |
| 148 | 0 | 0 | 0 | 0 | 0 | 0 | 0 | 0 | 0 | 0 | .11 | 0 | 0 | 0 | 0 | 0 | 0 | 0 | 0 | 0 | 0 |
| 149 | 0 | 0 | 0 | 0 | 0 | 0 | 0 | 0 | 0 | 0 | 0 | 0 | 0 | .11 | 0 | 0 | 0 | 0 | 0 | 0 | 0 |
| 150 | 0 | 0 | .11 | 0 | 0 | 0 | 0 | 0 | 0 | 0 | 0 | 0 | 0 | 0 | 0 | 0 | 0 | 0 | 0 | 0 | 0 |
| 151 | 0 | 0 | 0 | 0 | 0 | 0 | 0 | 0 | 0 | 0 | 0 | 0 | 0 | .11 | 0 | 0 | 0 | 0 | 0 | 0 | 0 |
| 152 | 0 | 0 | 0 | 0 | 0 | 0 | 0 | 0 | .11 | 0 | 0 | 0 | 0 | 0 | 0 | 0 | 0 | 0 | 0 | 0 | 0 |
| 153 | .11 | 0 | 0 | 0 | .11 | 0 | 0 | 0 | 0 | 0 | 0 | 0 | 0 | 0 | 0 | .11 | .11 | 0 | 0 | .11 | 0 |
| 154 | .11 | 0 | 0 | 0 | 0 | .11 | 0 | 0 | .11 | 0 | .11 | 0 | 0 | 0 | 0 | 0 | 0 | 0 | 0 | .11 | 0 |
| 155 | 0 | 0 | 0 | 0 | 0 | 0 | 0 | 0 | 0 | 0 | .11 | 0 | 0 | 0 | 0 | 0 | 0 | 0 | 0 | .11 | 0 |
| 156 | .11 | 0 | 0 | 0 | .11 | 0 | 0 | 0 | 0 | 0 | 0 | 0 | 0 | 0 | 0 | .11 | 0 | 0 | 0 | 0 | 0 |
| 157 | 0 | 0 | 0 | 0 | 0 | .11 | 0 | .11 | 0 | 0 | .11 | 0 | 0 | 0 | 0 | 0 | 0 | 0 | 0 | 0 | 0 |
| 158 | 0 | 0 | .11 | .11 | 0 | .11 | .11 | 0 | 0 | 0 | 0 | 0 | 0 | 0 | .11 | 0 | .11 | 0 | 0 | 0 | 0 |
| 159 | 0 | .11 | .11 | 0 | 0 | .11 | 1 | 0 | 0 | 0 | .11 | .11 | .11 | 0 | 0 | 0 | 0 | 0 | 0 | 0 | 0 |
| 160 | 0 | 0 | 0 | 0 | 0 | 0 | 0 | 0 | 0 | 0 | 0 | 0 | 0 | .11 | 0 | 0 | 0 | 0 | 0 | 0 | 0 |
| 161 | 0 | 0 | 0 | .11 | 0 | 0 | .11 | 0 | 0 | 1 | .11 | 0 | 0 | 0 | 0 | 0 | 0 | 0 | 0 | 0 | 0 |
| 162 | .11 | 1 | 0 | 0 | 0 | 0 | .11 | 0 | 0 | 0 | 0 | 0 | 0 | 0 | 0 | 0 | 0 | 0 | 0 | 0 | 0 |
| 163 | .11 | .11 | 0 | 0 | 0 | 0 | 0 | 0 | 0 | 0 | .11 | .11 | 0 | 0 | 0 | 0 | 0 | 0 | 0 | 0 | 0 |
| 164 | 0 | 0 | 0 | 0 | 0 | 0 | 0 | .11 | 0 | 0 | 0 | 0 | 0 | 0 | 0 | 0 | 0 | 0 | 0 | 0 | 0 |
| 165 | 0 | 0 | 0 | 0 | 0 | 0 | 0 | 0 | 0 | 1 | .11 | 0 | 0 | 0 | 0 | 0 | 0 | 0 | .11 | 0 | 0 |
| 166 | 0 | 0 | 0 | 0 | 0 | 0 | 0 | 0 | 0 | 0 | .11 | 0 | 0 | 0 | 0 | 0 | 0 | 0 | 0 | 0 | 0 |
| 167 | 0 | 0 | 0 | .11 | 0 | 0 | .11 | 0 | 0 | 0 | 0 | 0 | 0 | 0 | 0 | 0 | 0 | 0 | 0 | 0 | 0 |
| 168 | 0 | 0 | 0 | 0 | .11 | 0 | 0 | 0 | 0 | 0 | 0 | 0 | 0 | 0 | .11 | 0 | .11 | 0 | 0 | 0 | 0 |
| 169 | 0 | 0 | 1 | .11 | .11 | .11 | .11 | .11 | 0 | 0 | .11 | 0 | 0 | 0 | 0 | .11 | .11 | 0 | 0 | .11 | 0 |
| 170 | .11 | 0 | 1 | .11 | .11 | 0 | 0 | .11 | .11 | 0 | 0 | 0 | 0 | 0 | .11 | .11 | .11 | 0 | 0 | .11 | 0 |
| 171 | .11 | 0 | 0 | .11 | 0 | .11 | .11 | 0 | 0 | 0 | .11 | .11 | 0 | 0 | .11 | .11 | .11 | 0 | 0 | .11 | 0 |
| 172 | 0 | 0 | .11 | 0 | 0 | 0 | 1 | 0 | .11 | 0 | 0 | 1 | 0 | 0 | 0 | .11 | .11 | 1 | 0 | 0 | 0 |
| 173 | 1 | .11 | .11 | 1 | 0 | 0 | 0 | .11 | 0 | .11 | .11 | .11 | 0 | 0 | .11 | .11 | 0 | 0 | 0 | 0 | 0 |
| 174 | 0 | 0 | 0 | 1 | 0 | 0 | .11 | 0 | 0 | 0 | .11 | 0 | .11 | 0 | .11 | .11 | 0 | 0 | 0 | 0 | 0 |
| 175 | 0 | 0 | .11 | .11 | 0 | 0 | .11 | 0 | 0 | 0 | 0 | 0 | 0 | 0 | 0 | 0 | 0 | 0 | 0 | 0 | 0 |
| 176 | 1 | 0 | 0 | 0 | 0 | 0 | .11 | 0 | 0 | 0 | 0 | 1 | 0 | 0 | 0 | 0 | 0 | 0 | 0 | 0 | 0 |
| 177 | 0 | 0 | 0 | 0 | 0 | .11 | 0 | 0 | 0 | 0 | 0 | .11 | 0 | 0 | .11 | .11 | 0 | 0 | 0 | 0 | 0 |
| 178 | .11 | 0 | 0 | 0 | 0 | 0 | 0 | 0 | 0 | 0 | 0 | 0 | 0 | 0 | 0 | 0 | 0 | 0 | 0 | .11 | 0 |
| 179 | .11 | 0 | 0 | 0 | 0 | 0 | 1 | 0 | 0 | 0 | .11 | 0 | 0 | 0 | 1 | .11 | 0 | 0 | 0 | .11 | 0 |
| 180 | 0 | 0 | 0 | 0 | 0 | 0 | 0 | .11 | 0 | 0 | 0 | 0 | 0 | 0 | 0 | 0 | 0 | 0 | 0 | 0 | 0 |
| 181 | 0 | 0 | 0 | 0 | 0 | 0 | 0 | 0 | 0 | 0 | 0 | 0 | 0 | .11 | 0 | 0 | 0 | 0 | 0 | 0 | 0 |
| 182 | 0 | 0 | 0 | 0 | 0 | 0 | 0 | 0 | 0 | 0 | 0 | 0 | 0 | 0 | .11 | 0 | 0 | 0 | 0 | 0 | 0 |
| 183 | 0 | 0 | 0 | 0 | 0 | 1 | 1 | 0 | 1 | .11 | .11 | 0 | .11 | 0 | .11 | 0 | 0 | 1 | 1 | 0 | 0 |
| 184 | 0 | 0 | 0 | 0 | 0 | 0 | 0 | 0 | 0 | 0 | .11 | 0 | 0 | 0 | 0 | 0 | 0 | 0 | 0 | 0 | 0 |
| 185 | 0 | .11 | 0 | 0 | 0 | 0 | 0 | 0 | 0 | 0 | 0 | .11 | 0 | 0 | 0 | 0 | 0 | 0 | 0 | 0 | 0 |
| 186 | 0 | 0 | 0 | 0 | 0 | 0 | 0 | 0 | 0 | 0 | 0 | 0 | 0 | 0 | 0 | 0 | 0 | 0 | 0 | .11 | 0 |
| 187 | 1 | 1 | 0 | 0 | 0 | 0 | 0 | 0 | 0 | 0 | 0 | .11 | 0 | 0 | 0 | 0 | 0 | 0 | 0 | 0 | 0 |
| 188 | 0 | 0 | 0 | .11 | 0 | 0 | 0 | 0 | 0 | 0 | 0 | 0 | 0 | 0 | 0 | 0 | 0 | 0 | 0 | 0 | 0 |
| 189 | 0 | 0 | 0 | 0 | 0 | 0 | 0 | 0 | 0 | .11 | .11 | 0 | 0 | 0 | 0 | 0 | 0 | 0 | 0 | 0 | 0 |
| 190 | 0 | .11 | 0 | 0 | 0 | 0 | 0 | 0 | 0 | 0 | 0 | .11 | 0 | 0 | 0 | 0 | 0 | 0 | 0 | 0 | 0 |
| 191 | 0 | .11 | 0 | 0 | 0 | 0 | 0 | 0 | 0 | 0 | 0 | 0 | 0 | 0 | 0 | .11 | 0 | 0 | 0 | 0 | 0 |
| 192 | .11 | 0 | 0 | 0 | 0 | 0 | 0 | 0 | 0 | 0 | 0 | 0 | 0 | 0 | 0 | 0 | 0 | 0 | 0 | 0 | 0 |
| 193 | 0 | 0 | 0 | 0 | 0 | 0 | 0 | 0 | 0 | 0 | 0 | 0 | 0 | .11 | 0 | 0 | 0 | 0 | .11 | 0 | 0 |
| 194 | 0 | 0 | 0 | 0 | 0 | .11 | 0 | 0 | 0 | 0 | 0 | 0 | 0 | 0 | .11 | .11 | 0 | 0 | 0 | 0 | 0 |
| 195 | 0 | 0 | .11 | 0 | 0 | .11 | 0 | .11 | .11 | 0 | 0 | 0 | 0 | 0 | 0 | 0 | 0 | 0 | 0 | 0 | 0 |
| 196 | 0 | 0 | 0 | 0 | 0 | 0 | 0 | 0 | 0 | .11 | 0 | 0 | 0 | 0 | 0 | 0 | 0 | .11 | 0 | 0 | 0 |
| 197 | 0 | .11 | 0 | 0 | 0 | .11 | 0 | 0 | 0 | 0 | 0 | .11 | 0 | 0 | 0 | 0 | 0 | 0 | 0 | 0 | 0 |
| 198 | 0 | 0 | 0 | .11 | 0 | 0 | .11 | 0 | 0 | .11 | 0 | 0 | .11 | 0 | 0 | .11 | .11 | 0 | 0 | .11 | 0 |
| 199 | .11 | 0 | 0 | .11 | 0 | .11 | .11 | .11 | .11 | 0 | .11 | .11 | .11 | 0 | 0 | .11 | 0 | 0 | .11 | 0 | 0 |
| 200 | 0 | .11 | .11 | 0 | 0 | 0 | 0 | 0 | 0 | 0 | 0 | .11 | 0 | 0 | 0 | .11 | 0 | 0 | 0 | 0 | 0 |
| 201 | 0 | 0 | 0 | 0 | 0 | 0 | .11 | 0 | 0 | 0 | 0 | 0 | 0 | 0 | .11 | 0 | .11 | 0 | 0 | 0 | 0 |
| 202 | 0 | 0 | 0 | 0 | 0 | .11 | 0 | 0 | .11 | .11 | 0 | 0 | 0 | 0 | 0 | 0 | .11 | 0 | 0 | .11 | 0 |
| 203 | 0 | 0 | 0 | 0 | 0 | 0 | 0 | 0 | 0 | 0 | .11 | 0 | 0 | 0 | 0 | 0 | 0 | 0 | 0 | 0 | 0 |
| 204 | 0 | 0 | 0 | .11 | 0 | 0 | 1 | .11 | 0 | 0 | 0 | 0 | .11 | 0 | 0 | 0 | .11 | 0 | 0 | 0 | 0 |
| 205 | 0 | 0 | 0 | 0 | 0 | 0 | 0 | 0 | 0 | 0 | 0 | .11 | 0 | 0 | .11 | 0 | 0 | 0 | 0 | 0 | 0 |
| 206 | 0 | 0 | 0 | 0 | 0 | 0 | 0 | 0 | 0 | 0 | 0 | .11 | .11 | 0 | 0 | 0 | 0 | 0 | 0 | 0 | 0 |
| 207 | 0 | 0 | 0 | 0 | 0 | 0 | 0 | 0 | 0 | .11 | .11 | 0 | 0 | 0 | 0 | 0 | 0 | 0 | 0 | .11 | 0 |
| 208 | .11 | 0 | .11 | .11 | 0 | 0 | .11 | .11 | 0 | .11 | .11 | .11 | 0 | 0 | 0 | 0 | .11 | .11 | 0 | .11 | 0 |
| 209 | .11 | 0 | 0 | 0 | 0 | .11 | .11 | .11 | 0 | 0 | 0 | 0 | 0 | 0 | 0 | 0 | .11 | 0 | .11 | 0 | 0 |
| 210 | 0 | 0 | 0 | 0 | 0 | 0 | 0 | 0 | 0 | 0 | 0 | 0 | 0 | 0 | 0 | 0 | .11 | 0 | 0 | 0 | 0 |
| 211 | 0 | .11 | 0 | 0 | 0 | 0 | 0 | 0 | 0 | 0 | 0 | 1 | 0 | 0 | 0 | 0 | 0 | 0 | 0 | 0 | 0 |
| 212 | .11 | 1 | 1 | 0 | 0 | 0 | 1 | 0 | 0 | 0 | .11 | 0 | 0 | 0 | 0 | 0 | 0 | 0 | 0 | 0 | 0 |
| 213 | 0 | 0 | 0 | 0 | 0 | 0 | 0 | 0 | 0 | 0 | 0 | 0 | 0 | 0 | 0 | .11 | 0 | 0 | 0 | 0 | 0 |
| 214 | 0 | .11 | 0 | 0 | 0 | 0 | 0 | 0 | 0 | 0 | .11 | .11 | 0 | 0 | 0 | .11 | 0 | 0 | 0 | 0 | 0 |
| 215 | 0 | 0 | 0 | 0 | 0 | 0 | 0 | .11 | 0 | 0 | 0 | 0 | 0 | 0 | 0 | 0 | 0 | 0 | 0 | 0 | 0 |
| 216 | 0 | 0 | 0 | 0 | 0 | 0 | 0 | 0 | 0 | 0 | 0 | 0 | 0 | 0 | 0 | 0 | 0 | 0 | 0 | .11 | 0 |
| 217 | 0 | 0 | 0 | 0 | 0 | 0 | 0 | 0 | 0 | .11 | 0 | 0 | 0 | 0 | 0 | 0 | 0 | 0 | 0 | 0 | 0 |
| 218 | 0 | 0 | 0 | 0 | 0 | 0 | 0 | 0 | 0 | 0 | 0 | 0 | 0 | 0 | 0 | 0 | 0 | .11 | 0 | 0 | 0 |
| 219 | 0 | 0 | .11 | 0 | 0 | 0 | 0 | 0 | 0 | 0 | 0 | 0 | 0 | 0 | 0 | 0 | 0 | 0 | 0 | 0 | 0 |
| 220 | 0 | 0 | 0 | 0 | 0 | 0 | 0 | 0 | 0 | 0 | 0 | 0 | 0 | 0 | 0 | .11 | 0 | 0 | 0 | 0 | 0 |
| 221 | .11 | 0 | 0 | 0 | 0 | 0 | 0 | 0 | 0 | 0 | 0 | 0 | 0 | .11 | 0 | 0 | 0 | 0 | 0 | 0 | 0 |
| 222 | .11 | .11 | .11 | 0 | 0 | .11 | 0 | 0 | .11 | 0 | 0 | .11 | 0 | 0 | .11 | 0 | 0 | 0 | 0 | 0 | 0 |
| 223 | 0 | 0 | 0 | 0 | 0 | 0 | .11 | 0 | 0 | 0 | 0 | 0 | 0 | 0 | 0 | 0 | 0 | 0 | 0 | 0 | 0 |
| 224 | 0 | 0 | 0 | 0 | 0 | 0 | 0 | 0 | 0 | 0 | .11 | 0 | 0 | 0 | 0 | 0 | 0 | 0 | 0 | 0 | 0 |
| 225 | 0 | 0 | 0 | 0 | 0 | 0 | .11 | 0 | 0 | 0 | 0 | 0 | 0 | 0 | 0 | 0 | 0 | 0 | 0 | 0 | 0 |
| 226 | 0 | .11 | 0 | 0 | 0 | 0 | .11 | 0 | 0 | 0 | 0 | 0 | 0 | 0 | 0 | .11 | .11 | 0 | 0 | 0 | 0 |
| 227 | 0 | 0 | 0 | 0 | 0 | 0 | 0 | 0 | 0 | 0 | 0 | 0 | 0 | 0 | 0 | .11 | .11 | 0 | 0 | 0 | 0 |
| 228 | 0 | 0 | 0 | 0 | 0 | 0 | .11 | 0 | 0 | 0 | 0 | 0 | 0 | 0 | 0 | 0 | 0 | 0 | 0 | 0 | 0 |
| 229 | 0 | 0 | 0 | 0 | 0 | .11 | 0 | 0 | 0 | 0 | .11 | 0 | .11 | 0 | 0 | 0 | 0 | 0 | 0 | 0 | 0 |
| 230 | 0 | 0 | 0 | 0 | 0 | 0 | .11 | 0 | 0 | 0 | 0 | 0 | 0 | 0 | 0 | .11 | 0 | 0 | 0 | 0 | 0 |
| 231 | 0 | 0 | 0 | 0 | 0 | 0 | 0 | 0 | 0 | 0 | 0 | 0 | 0 | 0 | 0 | 0 | .11 | 0 | 0 | 0 | 0 |
| 232 | 0 | 0 | 0 | 0 | 0 | 0 | 0 | 0 | 0 | .11 | 0 | 0 | 0 | 0 | 0 | 0 | 0 | 0 | 0 | .11 | 0 |
| 233 | .11 | .11 | 0 | 0 | .11 | .11 | 0 | .11 | 0 | .11 | .11 | .11 | .11 | 0 | 0 | .11 | .11 | .11 | 0 | .11 | 0 |
| 234 | .11 | .11 | .11 | 0 | 0 | .11 | .11 | 0 | .11 | 0 | 0 | 0 | 0 | 0 | 0 | .11 | .11 | 0 | 0 | 0 | 0 |
| 235 | 0 | 0 | 0 | 0 | 0 | 0 | .11 | 0 | 0 | 0 | 0 | 0 | 0 | 0 | 0 | 0 | 0 | 0 | 0 | 0 | 0 |
| 236 | 0 | 0 | 0 | 0 | 0 | 0 | 0 | 0 | 0 | .11 | 0 | 0 | 0 | 0 | 0 | 0 | 0 | 0 | 0 | 0 | 0 |
| 237 | 0 | 0 | 0 | 0 | 0 | 0 | 0 | 0 | 0 | 0 | 0 | 0 | 0 | 0 | .11 | 0 | 0 | 0 | 0 | 0 | 0 |
| 238 | .11 | 0 | 0 | 0 | 0 | 0 | 0 | .11 | 0 | 0 | 0 | .11 | 0 | 0 | 0 | 0 | 0 | 0 | 0 | 0 | 0 |
| 239 | 0 | 0 | 0 | 0 | 0 | 0 | 0 | 0 | 0 | 0 | 0 | 0 | 0 | 0 | .11 | 0 | 0 | 0 | 0 | 0 | 0 |
| 240 | 0 | 0 | 0 | 0 | 0 | 0 | 0 | 0 | 0 | 0 | 0 | 0 | 0 | 0 | 0 | .11 | 0 | 0 | 0 | 0 | 0 |
| 241 | 0 | 0 | 0 | 0 | 0 | 0 | 0 | 0 | 0 | 0 | 0 | 0 | 0 | .11 | 0 | 0 | 0 | 0 | 0 | 0 | 0 |
| 242 | 0 | 0 | 0 | 0 | 0 | 0 | 0 | 0 | 0 | 0 | .11 | 0 | 0 | 0 | 0 | 0 | 0 | 0 | 0 | 0 | 0 |
| 243 | 0 | 0 | 0 | 0 | .11 | 0 | 0 | 0 | 0 | .11 | .11 | 0 | .11 | 0 | 0 | 0 | 0 | 0 | .11 | .11 | 0 |
| 244 | 0 | 0 | 0 | 0 | 0 | 0 | 0 | 0 | 0 | 0 | .11 | 0 | 0 | 0 | .11 | 0 | 0 | 0 | 0 | 0 | 0 |
| 245 | 0 | 0 | 0 | 0 | 0 | 0 | 0 | .11 | 0 | 0 | .11 | 0 | 0 | 0 | 0 | 0 | 0 | 0 | 0 | 0 | 0 |
| 246 | 0 | 0 | 0 | 0 | 0 | 0 | 0 | 0 | 0 | 0 | 0 | 0 | 0 | 0 | .11 | 0 | 0 | 0 | 0 | 0 | 0 |
| 247 | .11 | 0 | 0 | 0 | .11 | 0 | 0 | 0 | 0 | 0 | .11 | .11 | 0 | 0 | 0 | 0 | 0 | 0 | 0 | 0 | 0 |
| 248 | 0 | 0 | 0 | 0 | .11 | 0 | 0 | 0 | .11 | 0 | .11 | 0 | 0 | 0 | 0 | 0 | 0 | .11 | 0 | 0 | 0 |
| 249 | 0 | 0 | .11 | 0 | 0 | 0 | .11 | 0 | .11 | .11 | .11 | 0 | .11 | 0 | .11 | .11 | .11 | 0 | .11 | 0 | 0 |
| 250 | .11 | .11 | 0 | 0 | 0 | 0 | 0 | 0 | 0 | 0 | 0 | 0 | 0 | 0 | 0 | 0 | .11 | 0 | 0 | 0 | 0 |
| 251 | .11 | 0 | 0 | 0 | 0 | 0 | 0 | 0 | 0 | .11 | .11 | 0 | .11 | .11 | 0 | 0 | 0 | 0 | 0 | 0 | 0 |
| 252 | .11 | 0 | .11 | 0 | 0 | .11 | .11 | 0 | 0 | 0 | 0 | 0 | 0 | 0 | .11 | .11 | 0 | 0 | 0 | 0 | 0 |
| 253 | .11 | 0 | .11 | .11 | 0 | 0 | .11 | .11 | 0 | 0 | 0 | 0 | 0 | 0 | .11 | .11 | .11 | 0 | 0 | .11 | 0 |
| 254 | .11 | 0 | 0 | 0 | 0 | .11 | 0 | .11 | 0 | 0 | 0 | 0 | .11 | 0 | .11 | .11 | .11 | 0 | 0 | .11 | 0 |
| 255 | 0 | 0 | .11 | 0 | 0 | 0 | 0 | 0 | 0 | 0 | .11 | 0 | 0 | 0 | 0 | .11 | .11 | 0 | 0 | 0 | 0 |
| 256 | .11 | 0 | 0 | .11 | 0 | 0 | 0 | .11 | 0 | 0 | .11 | 0 | .11 | 0 | .11 | 0 | .11 | .11 | 0 | 0 | 0 |
| 257 | .11 | 0 | 0 | 0 | 0 | .11 | 0 | 0 | 0 | 0 | .11 | 0 | 0 | 0 | 0 | 0 | 0 | 0 | 0 | 0 | 0 |
| 258 | 0 | 0 | .11 | .11 | 0 | 0 | 0 | 0 | 0 | 0 | 0 | 0 | 0 | 0 | 0 | 0 | 0 | 0 | 0 | 0 | 0 |
| 259 | 0 | 0 | .11 | 0 | 0 | .11 | .11 | .11 | .11 | 0 | 0 | 0 | 0 | 0 | .11 | .11 | .11 | 0 | .11 | 0 | 0 |
| 260 | 0 | 0 | 0 | .11 | 0 | 0 | 0 | 0 | 0 | 0 | 0 | 0 | 0 | 0 | 0 | 0 | 0 | 0 | 0 | 0 | 0 |
| 261 | .11 | .11 | 0 | 0 | 0 | 0 | 0 | 0 | .11 | 0 | .11 | .11 | 0 | 0 | .11 | .11 | 0 | .11 | 0 | 0 | 0 |
| 262 | .11 | 0 | 0 | 0 | 0 | 0 | 0 | .11 | 0 | 0 | 0 | 0 | 0 | 0 | 0 | .11 | .11 | 0 | 0 | 0 | 0 |
| 263 | 0 | 0 | 0 | 0 | .11 | 0 | 0 | 0 | 0 | 0 | 0 | 0 | 0 | 0 | 0 | 0 | .11 | 0 | 0 | .11 | 0 |
| 264 | .11 | 0 | 0 | 0 | 0 | 0 | 0 | 0 | 0 | 0 | 0 | 0 | 0 | .11 | 0 | 0 | 0 | 0 | 0 | 0 | 0 |
| 265 | .11 | 0 | 0 | 1 | 0 | .11 | 0 | 0 | .11 | 0 | 0 | 0 | 0 | 0 | 0 | 0 | 0 | 0 | 0 | 0 | 0 |
| 266 | 0 | 0 | 0 | 0 | 0 | 0 | 0 | 0 | 0 | 0 | .11 | 0 | 0 | 0 | 0 | 0 | 0 | .11 | 0 | 0 | 0 |
| 267 | 0 | 0 | 0 | 0 | 0 | 0 | 0 | 0 | 0 | 0 | .11 | 0 | 0 | 0 | 0 | 0 | 0 | 0 | 0 | 0 | 0 |
| 268 | .11 | 0 | 0 | .11 | 0 | 0 | 0 | .11 | .11 | 0 | 0 | 0 | 0 | 0 | 0 | 0 | 0 | 0 | 0 | .11 | 0 |
| 269 | 0 | .11 | .11 | 0 | 0 | .11 | 0 | 1 | 0 | 0 | .11 | 0 | .11 | 0 | 0 | 0 | .11 | 0 | 0 | 0 | 0 |
| 270 | 0 | 0 | 0 | 0 | 0 | .11 | 0 | 0 | 0 | 0 | 0 | .11 | 0 | 0 | 0 | 0 | 0 | 0 | 0 | 0 | 0 |
| 271 | 1 | 0 | 0 | 0 | 0 | 0 | 0 | 0 | 0 | 0 | 0 | 0 | 0 | 0 | .11 | 0 | 0 | 0 | 0 | 0 | 0 |
| 272 | 0 | 0 | 0 | .11 | 0 | 0 | 1 | 0 | 0 | 0 | 0 | .11 | 0 | 0 | .11 | .11 | 0 | 0 | 0 | 0 | 0 |
| 273 | .11 | .11 | 0 | 0 | 0 | 0 | 1 | 1 | .11 | 0 | 0 | .11 | 0 | 0 | 0 | .11 | 0 | 0 | 0 | 0 | 0 |
| 274 | 0 | 0 | 0 | 0 | 0 | 0 | 0 | 0 | 0 | 0 | 0 | 0 | 0 | 0 | 0 | .11 | 0 | 0 | 0 | 0 | 0 |
| 275 | 0 | 0 | 0 | 0 | 0 | 0 | 0 | 0 | 0 | 0 | 0 | 0 | 0 | 0 | 0 | 0 | 0 | 0 | 0 | .11 | 0 |
| 276 | 0 | 0 | 0 | 0 | 0 | 0 | 0 | 0 | 0 | 0 | .11 | 0 | 0 | 0 | 0 | 0 | 0 | 0 | 0 | 0 | 0 |
| 277 | 0 | 0 | 0 | 0 | 0 | 0 | 0 | 0 | 0 | 0 | .11 | 0 | 0 | 0 | 0 | 0 | 0 | 0 | .11 | 0 | 0 |
| 278 | 0 | 0 | 0 | 0 | 0 | 0 | 0 | 0 | 0 | 0 | 0 | 0 | 0 | 0 | 0 | 0 | 0 | 0 | 0 | .11 | 0 |
| 279 | 0 | 0 | 0 | 0 | 0 | 0 | 0 | .11 | 0 | 0 | 0 | 0 | 0 | 0 | 0 | .11 | 0 | 0 | 0 | 0 | 0 |
| 280 | 0 | 0 | 0 | 0 | 0 | 0 | 0 | 0 | 0 | 0 | 0 | 0 | 0 | .11 | 0 | 0 | 0 | 0 | 0 | 0 | 0 |
| 281 | 0 | 0 | 0 | 0 | 0 | 0 | 0 | .11 | 0 | 0 | 0 | 0 | 0 | 0 | 0 | 0 | 0 | 0 | 0 | 0 | 0 |
| 282 | 0 | 0 | 0 | 0 | 0 | 0 | 0 | 0 | 0 | 0 | 0 | 0 | 0 | 0 | 0 | .11 | .11 | 0 | 0 | 0 | 0 |
| 283 | 0 | 0 | 0 | 0 | 0 | .11 | 0 | 0 | 0 | .11 | 0 | 0 | 0 | 0 | 0 | 0 | .11 | 0 | 0 | 0 | 0 |
| 284 | 0 | 0 | 0 | 0 | 0 | 0 | 0 | 0 | 0 | 0 | 0 | 0 | 0 | 0 | 0 | .11 | .11 | 0 | 0 | 0 | 0 |
| 285 | 0 | 0 | 0 | 0 | 0 | .11 | .11 | 0 | 0 | 0 | 0 | 0 | 0 | 0 | 0 | .11 | 0 | 0 | 0 | 0 | 0 |
| 286 | 0 | 0 | 0 | 0 | 0 | 0 | 0 | 0 | 0 | 1 | .11 | 0 | 0 | 0 | 0 | 0 | 0 | 0 | 0 | .11 | 0 |
| 287 | 0 | 0 | 0 | .11 | 0 | 0 | 1 | 0 | 0 | 0 | 0 | 0 | 0 | 0 | 0 | 0 | 1 | 0 | 0 | 0 | 0 |
| 288 | 0 | 1 | 0 | 0 | 0 | 0 | .11 | 0 | 0 | 0 | 1 | 0 | 0 | 0 | .11 | 0 | 0 | 0 | 0 | 0 | 0 |
| 289 | .11 | .11 | .11 | .11 | 0 | 0 | 1 | .11 | .11 | 0 | 0 | .11 | 0 | 0 | 0 | .11 | .11 | 0 | 0 | 0 | 0 |
| 290 | 0 | 0 | 0 | .11 | 0 | 0 | .11 | 0 | 0 | 0 | 0 | 0 | 0 | 0 | 0 | 0 | 1 | 0 | 0 | 0 | 0 |
| 291 | 1 | 0 | 0 | 0 | 0 | 0 | 0 | 0 | 0 | 0 | 0 | 0 | 0 | .11 | 0 | 0 | 0 | 0 | 0 | 0 | 0 |
| 292 | 0 | 0 | 0 | 0 | 0 | 0 | 0 | 0 | 0 | 0 | .11 | 1 | 0 | 0 | .11 | 0 | 0 | 0 | 0 | 0 | 0 |
| 293 | 1 | 1 | 1 | 0 | 0 | 0 | .11 | 0 | 0 | 0 | 0 | 1 | 0 | 0 | 0 | 0 | 1 | 0 | 0 | 0 | 0 |
| 294 | 0 | 0 | 0 | 0 | 0 | 0 | 0 | 0 | 0 | .11 | 1 | 0 | 0 | 0 | 0 | 0 | 0 | 0 | 0 | 0 | 0 |
| 295 | .11 | 0 | 0 | 0 | 0 | 0 | 0 | 0 | 0 | 0 | 0 | 0 | 0 | 0 | 0 | 0 | 0 | 0 | 0 | 1 | 0 |
| 296 | .11 | .11 | 0 | 1 | 0 | .11 | 1 | 0 | .11 | 0 | 0 | 0 | 0 | 0 | 0 | 0 | 0 | 0 | 0 | .11 | 0 |
| 297 | .11 | 0 | 0 | 0 | 0 | 0 | 0 | .11 | 0 | 0 | 0 | 0 | 0 | 0 | 0 | 0 | 0 | 0 | 0 | 0 | 0 |
| 298 | 0 | 0 | 0 | 0 | 0 | 0 | 0 | 0 | 0 | 0 | .11 | 0 | 0 | 0 | 0 | 0 | 0 | 0 | 0 | 0 | 0 |
| 299 | 0 | 0 | 0 | 0 | 0 | 0 | 0 | 0 | 0 | 0 | 0 | 1 | .11 | 0 | 0 | 0 | 0 | 0 | 0 | .11 | 0 |
| 300 | .11 | 1 | 0 | .11 | 0 | .11 | .11 | .11 | 0 | 0 | 1 | 1 | 0 | 0 | 0 | .11 | 0 | 0 | 0 | 0 | 0 |
| 301 | 0 | 0 | 0 | 0 | 0 | 0 | 0 | 0 | 0 | 0 | 0 | 0 | 0 | 0 | 0 | .11 | 0 | 0 | 0 | 0 | 0 |
| 302 | 0 | .11 | 0 | 0 | 0 | 0 | 0 | .11 | 0 | 0 | 0 | .11 | 0 | 0 | 0 | 0 | 0 | 0 | 0 | 0 | 0 |
| 303 | 0 | 0 | 0 | 0 | .11 | .11 | 0 | 0 | 0 | 1 | 1 | 0 | 0 | 0 | 0 | 0 | 0 | 0 | 0 | .11 | 0 |
| 304 | 0 | 0 | 0 | 0 | 0 | 0 | 0 | 0 | 0 | 0 | 0 | .11 | 0 | 0 | .11 | .11 | 0 | 0 | 0 | 0 | 0 |
| 305 | 0 | 0 | 0 | 0 | 0 | 0 | 0 | 0 | 0 | 0 | 0 | 0 | 0 | .11 | 0 | 0 | 0 | 0 | 0 | 0 | 0 |
| 306 | 0 | 0 | 0 | 0 | 0 | 0 | 0 | 0 | 0 | 0 | .11 | 0 | 0 | 0 | 0 | 0 | 0 | 0 | 0 | .11 | 0 |
| 307 | 0 | 0 | 0 | 0 | 0 | 0 | 0 | 0 | 0 | 0 | 0 | 0 | 0 | 0 | 0 | 0 | 0 | .11 | 0 | 0 | 0 |
| 308 | 0 | .11 | .11 | 0 | .11 | .11 | 0 | 0 | 0 | 0 | .11 | 0 | 0 | .11 | 0 | .11 | .11 | 0 | .11 | .11 | 0 |
| 309 | 0 | 0 | 0 | 0 | 0 | 0 | 0 | 0 | 0 | 0 | 0 | 0 | 0 | 0 | 0 | 0 | 0 | 0 | 0 | .11 | 0 |
| 310 | 0 | .11 | 0 | 0 | 0 | 0 | 0 | .11 | 0 | 0 | 0 | 0 | 0 | 0 | 0 | 0 | 0 | 0 | 0 | 0 | 0 |
| 311 | 0 | 0 | 0 | 0 | 0 | 0 | 0 | 0 | 0 | 0 | 0 | 0 | 0 | 0 | .11 | 0 | 0 | 0 | 0 | 0 | 0 |
| 312 | 0 | 0 | 0 | 0 | 0 | 0 | 0 | .11 | 0 | 0 | 0 | 0 | 0 | 0 | 0 | 0 | 0 | 0 | 0 | 0 | 0 |
| 313 | 0 | 0 | 0 | 0 | 0 | 0 | 0 | 0 | 0 | 0 | 0 | 0 | 0 | .11 | 0 | .11 | 0 | 0 | 0 | 0 | 0 |
| 314 | 0 | 0 | 0 | 0 | 0 | 0 | 0 | 0 | 0 | 0 | 0 | 0 | 0 | 0 | 0 | 0 | 0 | 0 | 0 | .11 | 0 |
| 315 | 0 | 0 | 0 | 0 | 0 | 0 | 0 | 0 | 0 | 0 | 0 | .11 | 0 | 0 | 0 | .11 | .11 | 0 | 0 | 0 | 0 |
| 316 | 0 | 0 | 0 | 0 | 0 | 0 | 0 | .11 | 0 | 0 | 0 | 0 | 0 | 0 | 0 | 0 | 0 | 0 | 0 | 0 | 0 |
| 317 | 0 | .11 | 0 | 0 | 0 | 1 | 1 | 0 | 1 | 0 | .11 | 0 | 0 | 0 | 0 | .11 | 0 | 0 | .11 | 0 | 0 |
| 318 | 1 | 0 | 0 | 1 | 0 | 0 | .11 | 0 | 0 | 0 | 0 | 0 | 0 | 0 | 0 | 0 | .11 | 0 | 0 | 0 | 0 |
| 319 | 0 | 0 | 0 | 0 | 0 | 0 | 0 | 0 | 0 | 0 | 0 | 0 | 0 | 0 | 0 | 0 | 0 | .11 | 0 | 0 | 0 |
| 320 | 0 | 0 | 0 | 0 | 0 | 0 | 0 | 0 | 0 | .11 | 0 | 0 | 0 | 0 | 0 | 0 | 1 | 0 | 0 | .11 | 0 |
| 321 | 0 | 0 | 0 | .11 | 0 | 0 | .11 | 0 | 0 | 0 | 0 | 0 | 0 | 0 | 0 | 0 | 0 | 0 | 0 | 0 | 0 |
| 322 | .11 | .11 | 0 | 0 | 0 | 0 | 0 | .11 | 0 | 1 | .11 | .11 | .11 | 1 | .11 | .11 | .11 | 0 | 0 | .11 | 0 |
| 323 | 0 | 0 | 0 | 0 | 0 | 0 | 0 | 0 | 0 | 1 | .11 | 0 | 0 | 0 | 0 | 0 | 0 | 0 | 0 | 0 | 0 |
| 324 | 0 | 0 | 0 | .11 | 0 | 0 | 0 | .11 | 0 | 0 | 0 | 0 | 0 | 0 | .11 | 0 | 0 | 0 | 0 | 0 | 0 |
| 325 | 0 | 0 | 0 | .11 | 0 | 0 | 1 | 0 | 0 | 0 | 0 | 0 | 0 | 0 | 1 | 0 | 0 | 0 | 0 | 0 | 0 |
| 326 | .11 | 0 | .11 | 0 | 0 | 0 | .11 | .11 | 0 | 0 | 0 | 0 | 0 | 0 | 0 | .11 | 0 | 0 | 0 | 0 | 0 |
| 327 | 0 | 0 | 0 | 0 | 0 | 0 | 0 | 0 | 0 | 0 | .11 | 0 | 0 | .11 | 0 | 0 | 0 | 0 | .11 | 0 | 0 |
| 328 | 0 | 0 | 0 | 0 | 0 | 0 | 0 | 0 | 0 | 0 | .11 | 0 | 0 | 1 | 0 | 0 | .11 | 0 | 0 | 0 | 0 |
| 329 | 0 | 0 | 0 | 0 | .11 | 0 | 0 | .11 | 0 | 0 | 0 | 0 | 0 | 0 | .11 | 0 | 0 | 0 | 0 | 0 | 0 |
| 330 | 0 | 0 | 0 | .11 | 0 | 0 | .11 | .11 | 0 | 0 | 0 | 0 | 0 | 0 | 1 | .11 | .11 | 0 | 0 | 0 | 0 |
| 331 | 0 | .11 | 0 | 0 | .11 | 1 | 0 | 0 | 0 | 0 | 1 | .11 | 0 | 0 | 0 | 0 | 0 | 0 | 0 | 0 | 0 |
| 332 | 0 | 0 | 0 | 0 | 0 | 0 | 0 | .11 | 0 | 0 | 0 | 0 | 0 | 0 | 0 | 0 | 0 | 0 | 0 | 0 | 0 |
| 333 | 0 | .11 | 0 | 0 | 0 | 0 | 0 | 0 | 0 | 0 | 0 | 0 | 0 | 0 | 0 | 0 | 0 | 0 | 0 | 0 | 0 |
| 334 | 0 | 0 | 0 | 0 | 0 | 0 | 0 | 0 | 0 | .11 | 0 | 0 | 0 | 0 | 0 | 0 | 0 | 0 | 0 | .11 | 0 |
| 335 | 0 | 0 | 0 | 0 | 0 | 0 | 0 | 0 | 0 | 0 | 0 | 0 | 0 | 0 | 0 | 0 | 0 | 0 | 0 | .11 | 0 |
| 336 | 0 | .11 | 0 | 0 | 0 | .11 | .11 | 0 | 0 | 0 | 0 | .11 | .11 | 0 | 0 | 0 | 0 | 0 | .11 | 0 | 0 |
| 337 | 0 | 0 | .11 | 0 | 0 | 0 | 0 | 0 | 0 | 0 | 0 | 0 | 0 | .11 | 0 | .11 | 0 | .11 | 0 | 0 | 0 |
| 338 | .11 | 0 | 0 | 0 | .11 | 0 | 0 | 0 | 0 | 0 | 0 | 0 | 0 | 0 | 0 | 0 | 0 | 0 | 0 | .11 | 0 |
| 339 | 0 | 0 | 0 | .11 | 0 | 0 | 0 | 0 | 0 | 0 | 0 | 0 | 0 | 0 | .11 | 0 | 0 | 0 | 0 | 0 | 0 |
| 340 | 0 | 0 | 0 | 0 | 0 | .11 | 0 | 0 | 0 | 0 | 0 | 0 | 0 | 0 | 0 | 0 | 0 | 0 | 0 | 0 | 0 |
| 341 | 0 | 0 | 0 | 0 | 0 | .11 | 0 | 0 | 0 | 0 | 0 | 0 | 0 | 0 | 0 | 0 | 0 | 0 | 0 | .11 | 0 |
| 342 | 0 | .11 | 0 | 0 | 0 | 0 | .11 | 0 | 0 | 0 | 0 | 0 | 0 | 0 | 0 | 0 | 0 | .11 | 0 | 0 | 0 |
| 343 | 0 | 0 | 0 | 0 | 0 | 0 | 0 | 0 | 0 | .11 | 0 | 0 | 0 | 0 | 0 | 0 | 0 | 0 | 0 | .11 | 0 |
| 344 | 0 | 0 | 0 | 0 | 0 | 0 | 0 | 0 | 0 | 0 | .11 | 0 | 0 | 0 | 0 | 0 | 0 | 0 | 0 | 0 | 0 |
| 345 | .11 | 0 | 0 | 0 | 0 | 0 | 0 | 0 | 0 | 0 | 0 | 0 | 0 | 0 | 0 | 0 | 0 | 0 | 0 | 0 | 0 |
| 346 | 0 | 0 | 0 | 0 | 0 | 0 | 0 | 0 | .11 | 0 | 0 | 0 | 0 | 0 | 0 | 0 | 0 | 0 | 0 | 0 | 0 |
| 347 | 0 | .11 | 0 | .11 | 0 | 0 | 0 | .11 | .11 | 0 | 0 | .11 | 0 | 0 | .11 | 0 | 0 | 0 | 0 | 0 | 0 |
| 348 | .11 | 0 | 0 | 0 | .11 | 0 | 0 | 0 | 0 | 0 | 0 | 0 | 0 | 0 | 0 | .11 | 0 | 0 | 0 | 0 | 0 |
| 349 | 0 | 0 | 0 | 0 | 0 | 0 | 0 | 0 | 0 | .11 | 0 | 0 | 0 | 0 | 0 | 0 | 0 | 0 | 0 | .11 | 0 |
| 350 | 0 | 0 | 0 | 0 | 0 | 0 | 0 | .11 | 0 | 0 | 0 | 0 | 0 | 0 | 0 | 0 | 0 | 0 | 0 | 0 | 0 |
| 351 | .11 | 0 | 0 | 0 | 0 | 0 | 0 | .11 | 0 | 0 | 0 | 0 | 0 | 0 | 0 | 0 | 0 | 0 | 0 | 0 | 0 |
| 352 | 0 | 0 | 0 | 0 | 0 | .11 | 0 | 0 | 0 | 0 | 0 | 0 | 0 | .11 | 0 | 0 | 0 | 0 | 0 | 0 | 0 |
| 353 | 0 | 0 | 0 | 0 | 0 | 0 | 0 | 0 | 0 | 0 | 0 | 0 | 0 | 0 | 0 | 0 | 0 | .11 | .11 | 0 | 0 |
| 354 | 0 | 0 | 0 | 0 | 0 | 0 | 0 | 0 | 0 | 0 | 0 | 0 | 0 | 0 | 0 | 0 | .11 | 0 | 0 | 0 | 0 |
| 355 | 0 | 0 | 0 | 0 | 0 | 0 | 0 | 0 | .11 | 0 | 0 | 0 | 0 | 0 | 0 | 0 | 0 | 0 | 0 | 0 | 0 |
| 356 | 0 | 0 | 0 | 0 | 0 | 0 | 0 | .11 | 0 | 0 | 0 | 0 | 0 | 0 | 0 | .11 | 0 | 0 | 0 | 0 | 0 |
| 357 | 0 | 0 | 0 | 0 | 0 | 0 | 0 | .11 | 0 | 0 | 0 | 0 | 0 | 0 | 0 | 0 | 0 | 0 | 0 | 0 | 0 |
| 358 | 0 | 0 | 0 | 0 | 0 | 0 | 0 | 0 | 0 | 0 | 0 | 0 | 0 | 0 | 0 | 0 | 0 | .11 | 0 | 0 | 0 |
| 359 | 0 | 0 | .11 | 0 | 0 | 0 | 0 | .11 | 0 | 0 | 0 | 0 | 0 | 0 | 0 | 0 | 0 | 0 | 0 | 0 | 0 |
| 360 | 0 | 0 | 0 | 0 | 0 | 0 | 0 | .11 | 0 | 0 | 0 | 0 | 0 | 0 | 0 | .11 | 0 | 0 | 0 | 0 | 0 |
| 361 | 0 | 0 | 0 | 0 | .11 | 0 | 0 | 0 | 0 | 0 | 0 | 0 | 0 | 0 | 0 | 0 | .11 | 0 | 0 | 0 | 0 |
| 362 | 0 | 0 | 0 | 0 | 0 | 0 | 0 | 0 | 0 | 0 | .11 | 0 | .11 | 0 | 0 | 0 | .11 | 0 | 0 | 0 | 0 |
| 363 | 0 | 0 | 0 | 0 | 0 | 0 | .11 | 0 | 0 | 0 | 0 | 0 | 0 | 0 | 0 | 0 | 0 | 0 | 0 | 0 | 0 |
| 364 | .11 | 0 | 0 | 0 | 0 | 0 | 0 | .11 | 0 | 0 | 0 | 0 | 0 | 0 | 0 | .11 | 0 | 0 | 0 | 0 | 0 |
| 365 | 0 | 0 | 0 | 0 | 0 | 0 | 0 | 0 | 0 | .11 | 0 | 0 | 0 | 0 | 0 | 0 | 0 | 0 | 0 | .11 | 0 |
| 366 | .11 | 0 | 0 | 0 | .11 | 0 | 0 | 0 | 0 | 0 | .11 | 0 | 0 | 0 | 0 | .11 | .11 | 0 | 0 | .11 | 0 |
| 367 | 0 | 0 | 0 | 0 | 0 | 0 | .11 | 0 | 0 | 0 | 0 | 0 | 0 | 0 | 0 | 0 | 0 | 0 | 0 | 0 | 0 |
| 368 | 0 | 0 | 0 | 0 | 0 | 0 | 0 | .11 | 0 | 0 | 0 | 0 | 0 | 0 | 0 | 0 | 0 | 0 | 0 | 0 | 0 |
| 369 | 0 | 0 | 0 | 0 | 0 | 0 | 0 | 0 | 0 | 0 | 0 | .11 | 0 | 0 | 0 | 0 | 0 | 0 | 0 | .11 | 0 |
| 370 | 0 | 0 | 0 | 0 | 0 | 0 | 0 | 0 | 0 | 0 | 0 | 0 | 0 | 0 | .11 | 0 | 0 | 0 | 0 | 0 | 0 |
| 371 | 0 | 0 | 0 | 0 | 0 | 0 | 0 | 0 | 0 | 0 | 0 | 0 | .11 | 0 | 0 | 0 | 0 | 0 | 0 | 0 | 0 |
| 372 | 0 | 0 | 0 | 0 | 0 | 0 | 0 | 0 | 0 | .11 | 0 | 0 | 0 | 0 | 0 | 0 | 0 | 0 | 0 | 0 | 0 |
| 373 | 0 | 0 | 0 | 0 | 0 | 0 | 0 | 0 | 0 | 0 | 0 | 0 | 0 | .11 | 0 | 0 | 0 | 0 | 0 | 0 | 0 |
| 374 | 0 | .11 | 0 | 0 | 0 | 0 | 0 | 0 | 0 | 0 | 0 | .11 | 0 | 0 | 0 | .11 | .11 | 0 | 0 | 0 | 0 |
| 375 | .11 | 0 | 0 | .11 | 0 | 0 | 0 | 0 | 0 | 0 | 0 | 0 | 0 | 0 | .11 | 0 | .11 | 0 | 0 | .11 | 0 |
| 376 | 0 | 0 | 0 | 0 | 0 | 0 | 0 | 0 | 0 | 0 | 0 | 0 | 0 | .11 | 0 | 0 | 0 | 0 | 0 | 0 | 0 |
| 377 | 0 | 0 | 0 | 0 | 0 | 0 | 0 | .11 | 0 | 0 | 0 | 0 | 0 | 0 | 0 | 0 | 0 | 0 | 0 | 0 | 0 |
| 378 | 0 | 0 | 0 | 0 | 0 | 0 | 0 | 0 | 0 | 0 | .11 | 0 | 0 | 0 | 0 | 0 | 0 | 0 | 0 | 0 | 0 |
| 379 | 0 | 0 | 0 | .11 | 0 | 0 | 0 | 0 | 0 | 0 | 0 | 0 | 0 | 0 | 0 | 0 | 0 | 0 | 0 | 0 | 0 |
| 380 | 0 | 0 | 0 | 0 | 0 | .11 | 0 | 0 | 0 | 0 | 0 | 0 | 0 | 0 | 0 | 0 | 0 | 0 | 0 | 0 | 0 |
| 381 | 0 | 0 | 0 | 0 | 0 | 0 | 0 | 0 | 0 | 0 | 0 | 0 | 0 | 0 | .11 | 0 | 0 | 0 | 0 | 0 | 0 |
| 382 | 0 | 0 | 0 | 0 | 0 | 0 | 0 | 0 | 0 | 0 | .11 | 0 | 0 | 0 | 0 | 0 | 0 | 0 | 0 | 0 | 0 |
| 383 | 0 | 0 | .11 | 0 | 0 | 0 | 0 | 0 | 0 | 0 | 0 | 0 | 0 | 0 | 0 | 0 | 0 | 0 | 0 | .11 | 0 |
| 384 | .11 | 0 | 0 | 0 | 0 | 0 | 0 | 0 | 0 | 0 | 0 | 0 | 0 | 0 | 0 | 0 | 0 | 0 | 0 | 0 | 0 |
| 385 | 0 | .11 | 0 | 0 | 0 | 0 | 0 | 0 | 0 | 0 | 0 | 0 | 0 | 0 | 0 | 0 | 0 | 0 | 0 | 1 | 0 |
| 386 | 0 | 0 | 0 | 0 | 0 | 0 | 0 | 0 | 0 | 0 | 0 | 0 | 0 | 0 | 0 | 0 | 0 | 0 | .11 | 0 | 0 |
| 387 | 0 | 0 | 0 | 0 | 0 | 0 | 0 | 0 | 0 | 0 | 0 | 0 | .11 | 0 | 0 | 0 | 0 | 0 | 0 | 0 | 0 |
| 388 | 0 | 0 | 0 | 0 | 0 | 0 | 0 | 0 | 0 | 0 | 0 | 0 | 0 | 0 | 0 | .11 | .11 | 0 | 0 | .11 | 0 |
| 389 | .11 | 0 | 1 | .11 | 0 | 0 | .11 | 0 | 0 | 0 | .11 | 1 | 0 | 0 | 0 | .11 | 0 | 0 | 0 | 0 | 0 |
| 390 | 0 | .11 | 0 | 0 | 0 | 0 | 0 | 0 | 0 | .11 | 0 | 1 | 0 | 0 | 0 | 0 | 0 | 0 | 0 | .11 | 0 |
| 391 | 0 | .11 | 0 | 0 | .11 | 0 | 0 | 0 | 0 | 0 | .11 | 1 | 0 | 0 | 0 | 0 | 0 | 0 | 0 | 0 | 0 |
| 392 | 0 | 0 | 0 | .11 | 0 | .11 | 0 | 0 | .11 | 0 | 0 | .11 | 0 | 0 | 0 | 0 | 0 | 0 | 0 | 0 | 0 |
| 393 | 0 | 0 | 0 | 0 | 0 | 0 | 0 | 0 | 0 | .11 | 0 | 0 | 0 | 0 | 0 | 0 | 0 | 0 | 0 | .11 | 0 |
| 394 | 0 | 0 | 0 | 0 | 0 | 0 | 0 | .11 | 0 | 0 | 0 | 0 | 0 | 0 | 0 | 0 | 0 | 0 | 0 | 0 | 0 |
| 395 | 0 | 0 | 0 | 0 | 0 | 0 | 0 | 0 | 0 | .11 | 0 | 0 | 0 | 0 | 0 | 0 | 0 | 0 | 0 | .11 | 0 |
| 396 | 0 | .11 | 0 | 0 | 0 | 0 | 0 | 0 | 0 | 0 | 0 | 0 | 0 | 0 | 0 | 0 | .11 | 0 | .11 | .11 | 0 |
| 397 | 0 | 0 | 0 | 0 | 0 | 0 | 0 | 0 | 0 | 0 | .11 | 0 | 0 | 0 | 0 | 0 | 0 | 0 | 0 | 0 | 0 |
| 398 | 0 | 1 | 0 | 1 | 0 | 0 | .11 | 0 | 0 | 0 | 0 | 0 | 0 | 0 | 0 | .11 | 0 | 0 | 0 | 0 | 0 |
| 399 | 0 | 1 | .11 | .11 | 0 | .11 | 0 | 0 | 0 | 0 | 0 | 0 | 0 | 0 | .11 | .11 | 0 | 0 | 0 | 0 | 0 |
| 400 | 0 | 0 | 0 | 0 | 0 | 0 | 0 | .11 | 0 | 0 | 0 | 0 | 0 | 0 | 0 | 0 | 0 | 0 | 0 | 0 | 0 |
| 401 | 0 | 0 | 0 | 0 | 0 | 0 | 0 | 0 | 0 | 0 | .11 | 0 | 0 | .11 | 0 | 0 | 0 | .11 | 0 | 0 | 0 |
| 402 | 0 | 0 | 1 | 1 | 0 | 0 | .11 | 0 | 0 | .11 | .11 | 0 | 0 | 0 | 0 | 0 | 0 | 0 | 0 | 1 | 0 |
| 403 | 0 | .11 | 0 | 0 | 0 | 0 | 0 | 0 | 0 | 0 | 0 | 1 | 0 | 0 | 1 | 0 | 1 | 0 | 0 | .11 | 0 |
| 404 | 1 | 0 | 0 | 1 | 0 | 0 | 1 | .11 | 0 | 0 | 0 | 0 | .11 | 0 | 0 | 0 | .11 | 0 | 0 | .11 | 0 |
| 405 | .11 | 1 | 0 | .11 | 0 | .11 | .11 | 0 | 0 | 0 | 0 | 1 | 0 | 0 | .11 | .11 | .11 | 0 | 0 | 0 | 0 |
| 406 | 0 | 0 | 0 | 0 | 0 | 0 | 0 | 0 | 0 | .11 | 0 | 0 | 0 | 0 | 0 | 0 | 0 | 0 | 0 | 0 | 0 |
| 407 | .11 | 0 | 0 | 0 | .11 | 0 | .11 | 0 | 0 | .11 | 0 | 0 | 0 | 0 | 0 | 0 | .11 | 0 | 0 | .11 | 0 |
| 408 | 1 | .11 | .11 | .11 | .11 | 0 | 1 | 0 | 0 | 0 | 0 | .11 | 0 | 0 | 0 | .11 | .11 | 0 | 0 | 0 | 0 |
| 409 | .11 | 0 | 0 | 0 | 0 | 0 | 0 | 0 | 0 | 0 | 0 | 0 | 0 | 0 | 0 | .11 | 0 | 0 | 0 | 0 | 0 |
| 410 | 0 | 0 | 0 | 0 | 0 | 0 | 0 | 0 | 0 | .11 | 0 | 0 | 0 | 0 | 0 | 0 | 0 | 0 | 0 | .11 | 0 |
| 411 | 0 | .11 | 0 | 0 | 0 | .11 | .11 | 0 | .11 | 0 | 0 | .11 | 0 | 0 | 0 | .11 | .11 | 0 | 0 | 0 | 0 |
| 412 | .11 | .11 | 0 | 0 | 0 | 0 | 1 | 0 | .11 | 0 | 1 | .11 | 0 | 0 | 0 | .11 | .11 | 0 | 1 | 0 | 0 |
| 413 | 0 | 0 | 0 | 0 | 0 | 0 | 0 | 0 | 0 | 0 | .11 | 0 | .11 | 0 | 0 | 0 | 0 | 0 | 0 | .11 | 0 |
| 414 | 0 | 0 | 0 | 0 | 0 | 0 | 0 | 0 | 0 | 0 | .11 | 0 | .11 | 0 | 0 | 0 | 0 | 0 | 0 | 0 | 0 |
| 415 | 0 | 1 | 0 | 0 | 0 | 0 | 1 | 0 | 0 | 0 | 0 | 0 | 0 | 0 | 0 | 0 | 1 | 0 | 0 | .11 | 0 |
| 416 | 1 | 0 | 0 | .11 | 0 | 0 | .11 | 0 | 0 | 0 | .11 | 0 | 0 | 0 | 0 | .11 | 0 | 0 | 0 | 0 | 0 |
| 417 | .11 | .11 | 0 | 0 | 0 | .11 | .11 | 0 | 0 | 0 | 0 | 0 | 0 | 0 | .11 | 0 | 0 | 0 | 0 | .11 | 0 |
| 418 | 1 | 1 | 0 | .11 | 0 | 0 | .11 | 0 | 0 | 0 | 0 | 1 | 0 | 0 | 0 | 1 | 0 | 0 | 0 | 0 | 0 |
| 419 | 0 | 0 | 0 | 0 | 0 | 0 | 0 | .11 | 0 | 0 | 0 | 0 | 0 | 0 | 0 | 0 | 0 | 0 | 0 | 0 | 0 |
| 420 | .11 | .11 | 0 | 0 | 0 | 0 | .11 | .11 | 0 | 0 | 0 | 1 | 0 | 0 | 0 | 0 | 0 | 0 | 0 | 0 | 0 |
| 421 | 0 | 0 | 0 | 0 | 0 | 0 | 1 | 0 | 0 | .11 | 0 | 0 | 0 | 0 | 1 | 0 | 0 | 0 | .11 | .11 | 0 |
| 422 | 0 | 0 | 0 | 0 | 0 | .11 | 0 | 0 | 0 | .11 | 0 | 0 | .11 | 0 | 0 | 0 | 0 | 0 | 1 | 0 | 0 |
| 423 | 0 | .11 | 0 | 0 | 0 | 0 | 0 | 0 | 0 | 0 | 0 | .11 | 0 | 0 | 0 | 0 | .11 | 0 | 0 | 0 | 0 |
| 424 | .11 | 0 | 1 | 0 | 0 | .11 | .11 | 0 | 0 | 0 | 0 | 1 | 0 | 0 | 0 | 0 | 0 | .11 | 0 | 0 | 0 |
| 425 | 0 | 0 | .11 | 0 | 0 | 0 | 0 | 0 | 0 | 0 | 0 | 0 | 0 | 0 | 0 | 0 | 0 | 0 | 0 | 0 | 0 |
| 426 | .11 | 0 | 0 | 0 | 0 | 0 | 0 | 0 | 0 | .11 | 0 | 0 | 0 | 0 | 0 | .11 | .11 | 0 | 0 | .11 | 0 |
| 427 | .11 | .11 | 0 | 0 | 0 | 0 | 0 | 0 | 0 | 0 | .11 | .11 | 0 | 0 | 0 | 0 | 0 | 0 | 0 | 0 | 0 |
| 428 | 0 | .11 | .11 | 0 | 0 | .11 | .11 | .11 | 0 | .11 | .11 | .11 | 0 | .11 | 0 | .11 | .11 | 0 | .11 | .11 | 0 |
| 429 | 0 | 0 | 0 | 0 | 0 | 0 | 0 | 0 | 0 | 0 | .11 | 0 | 0 | 0 | 0 | 0 | 0 | .11 | 0 | 0 | 0 |
| 430 | 0 | .11 | 0 | 0 | 0 | .11 | 0 | 0 | 0 | 0 | 0 | .11 | 0 | 0 | 0 | 0 | 0 | 0 | 0 | 0 | 0 |
| 431 | 1 | .11 | 0 | .11 | 0 | .11 | .11 | 0 | 0 | 0 | 0 | 0 | .11 | 0 | 0 | 0 | 0 | 0 | 0 | 0 | 0 |
| 432 | 0 | 0 | 0 | 0 | 0 | 0 | 0 | 0 | 0 | 0 | .11 | .11 | 0 | 0 | 0 | 0 | .11 | 0 | 0 | 0 | 0 |
| 433 | .11 | 0 | 0 | 0 | 0 | 0 | 0 | 0 | 0 | 0 | .11 | 0 | 0 | 0 | 0 | 0 | 0 | 0 | 0 | 0 | 0 |
| 434 | 0 | 0 | .11 | .11 | 0 | 0 | .11 | .11 | 0 | 0 | 0 | 0 | 0 | 0 | 0 | 0 | 0 | 0 | 0 | 0 | 0 |
| 435 | .11 | .11 | .11 | .11 | .11 | .11 | .11 | .11 | 0 | .11 | .11 | .11 | .11 | 0 | 0 | .11 | .11 | 0 | .11 | .11 | 0 |
| 436 | 0 | 0 | 0 | 0 | 0 | 0 | 0 | 0 | 0 | 0 | 0 | 0 | 0 | 0 | 0 | .11 | 0 | 0 | 0 | 0 | 0 |
| 437 | 0 | 0 | 0 | 0 | 0 | 0 | 0 | 0 | 0 | .11 | .11 | 0 | 0 | 0 | 0 | 0 | .11 | 0 | 0 | .11 | 0 |
| 438 | .11 | .11 | 0 | 0 | 0 | .11 | .11 | 0 | 0 | 0 | 0 | 0 | .11 | 0 | 0 | .11 | 0 | 0 | 0 | 0 | 0 |
| 439 | .11 | .11 | .11 | .11 | 0 | .11 | .11 | .11 | .11 | 0 | 0 | .11 | .11 | 0 | .11 | 0 | 0 | .11 | 0 | .11 | 0 |
| 440 | 0 | 0 | 0 | 0 | 0 | 0 | 0 | .11 | 0 | 0 | 0 | 0 | 0 | 0 | 0 | 0 | 0 | 0 | 0 | 0 | 0 |
| 441 | 0 | 0 | 0 | 0 | 0 | 0 | 0 | .11 | 0 | 0 | 0 | 0 | 0 | 0 | 0 | 0 | 0 | 0 | 0 | 0 | 0 |
| 442 | 0 | 0 | 0 | 0 | 0 | 0 | 0 | 0 | 0 | 0 | 0 | 0 | 0 | .11 | 0 | .11 | .11 | 0 | 0 | 0 | 0 |
| 443 | .11 | 0 | 0 | 0 | 0 | 0 | 0 | 0 | 0 | 0 | 0 | 0 | 0 | 0 | 0 | .11 | 0 | 0 | 0 | 0 | 0 |
| 444 | .11 | 0 | 0 | 0 | 0 | 0 | 0 | 0 | .11 | 0 | 0 | 0 | 0 | 0 | 0 | 0 | 0 | 0 | .11 | 0 | 0 |
| 445 | 0 | 0 | 0 | 0 | 0 | 0 | .11 | 0 | 0 | 0 | 0 | 0 | 0 | 0 | 0 | 0 | .11 | 0 | 0 | 0 | 0 |
| 446 | 0 | 0 | 0 | 0 | 0 | 0 | 0 | 0 | 0 | 0 | 0 | 0 | 0 | .11 | 0 | .11 | 0 | 0 | 0 | 0 | 0 |
| 447 | 0 | 0 | 0 | 0 | 0 | 0 | 0 | 0 | 0 | 0 | .11 | 0 | 0 | 0 | 0 | 0 | 0 | 0 | 0 | 0 | 0 |
| 448 | 0 | 0 | 0 | 0 | 0 | .11 | .11 | 0 | .11 | 0 | 0 | .11 | 0 | 0 | 0 | 0 | 0 | 0 | 0 | 0 | 0 |
| 449 | 0 | .11 | 0 | 0 | 0 | 0 | 0 | .11 | 0 | 0 | .11 | 0 | 0 | 0 | 0 | .11 | 0 | 0 | 0 | 0 | 0 |
| 450 | 0 | 0 | 0 | 0 | 0 | 0 | 0 | 0 | 0 | 0 | .11 | 0 | 0 | 0 | 0 | 0 | 0 | 0 | 0 | 0 | 0 |
| 451 | 0 | 0 | 0 | 0 | 0 | .11 | .11 | 0 | 0 | 0 | 0 | 0 | 0 | 0 | 0 | 0 | 0 | 0 | 0 | .11 | 0 |
| 452 | 0 | 0 | 0 | 0 | 0 | 0 | 0 | 0 | 0 | 0 | 0 | 0 | 0 | 0 | 0 | .11 | .11 | 0 | 0 | 0 | 0 |
| 453 | 0 | .11 | 0 | 0 | 0 | 0 | 0 | 0 | 0 | 0 | 0 | 0 | 0 | 0 | 0 | 0 | 0 | 0 | .11 | 0 | 0 |
| 454 | 0 | 0 | 0 | 0 | 0 | 0 | 0 | 0 | 0 | .11 | 0 | 0 | 0 | 0 | 0 | 0 | 0 | 0 | 0 | .11 | 0 |
| 455 | 0 | .11 | 0 | 0 | 0 | 0 | 0 | 0 | 0 | 0 | 0 | 0 | .11 | 0 | 0 | .11 | 0 | 0 | 0 | 0 | 0 |
| 456 | 0 | .11 | 0 | 0 | 0 | 0 | 0 | 0 | 0 | 0 | 0 | .11 | 0 | 0 | 0 | .11 | 0 | 0 | 0 | 0 | 0 |
| 457 | 0 | 0 | 0 | 0 | 0 | 0 | 0 | 0 | 0 | 0 | .11 | 0 | 0 | 0 | 0 | 0 | 0 | 0 | 0 | 0 | 0 |

TABLE 10
Position Specific Scoring Matrix (PSSM) of Successful B13GT Designs

| Seq | Amino Acid | | | | | | | | | | | | | | | | | | | | |
|---|---|---|---|---|---|---|---|---|---|---|---|---|---|---|---|---|---|---|---|---|---|
| Pos | A | R | N | D | C | Q | E | G | H | I | L | K | M | F | P | S | T | W | Y | V | — |
| 1 | 0 | 0 | 0 | 0 | 0 | 0 | 0 | 0 | 0 | 0 | 0 | 0 | .12 | 0 | 0 | 0 | 0 | 0 | 0 | 0 | 0 |
| 2 | 0 | .12 | .12 | 0 | 0 | .12 | .12 | .12 | .12 | 0 | .12 | .12 | .12 | .12 | .12 | 0 | 0 | 0 | 0 | .12 | 0 |
| 3 | 0 | 0 | .12 | 0 | 0 | 0 | 0 | 0 | 0 | 0 | .12 | 0 | .12 | .12 | 0 | .12 | 0 | 0 | 0 | 0 | 0 |
| 4 | 0 | 0 | .12 | 0 | 0 | 0 | 0 | 0 | 0 | 0 | 0 | .12 | 0 | 0 | .12 | .12 | .12 | 0 | 0 | 0 | 0 |
| 5 | .12 | 0 | .12 | 0 | 0 | .12 | 0 | 0 | .12 | 0 | .12 | 0 | .12 | 0 | 0 | .12 | .12 | 0 | 0 | 0 | 0 |
| 6 | 0 | 0 | 0 | 0 | 0 | 0 | .12 | 0 | 0 | 0 | 0 | 0 | 0 | 0 | .12 | 0 | 1 | 0 | 0 | 0 | 0 |
| 7 | .12 | 0 | 0 | 0 | 0 | .12 | 0 | 0 | 0 | 0 | .12 | 0 | 0 | 0 | .12 | 0 | .12 | 0 | 0 | 0 | 0 |
| 8 | 0 | 0 | 0 | .12 | 0 | 0 | 0 | 0 | 0 | 0 | .12 | 0 | 0 | 0 | 0 | 0 | .12 | 0 | 0 | 1 | 0 |
| 9 | .12 | 1 | 0 | 0 | 0 | 0 | 0 | .12 | 0 | 0 | .12 | .12 | .12 | 0 | 0 | .12 | 0 | 0 | 0 | .12 | 0 |
| 10 | 0 | .12 | 0 | 0 | 0 | 0 | 0 | 0 | 0 | 0 | 0 | .12 | 0 | 0 | 0 | 0 | 0 | 0 | 0 | 0 | 0 |
| 11 | 0 | .12 | 0 | 1 | 0 | 0 | 0 | 0 | 0 | 0 | 0 | 0 | 0 | 0 | 0 | 1 | 1 | 0 | 0 | 0 | 0 |
| 12 | 0 | .12 | 0 | 1 | 0 | .12 | 0 | 0 | 0 | 0 | 0 | 0 | 0 | 0 | 0 | .12 | 0 | 0 | 0 | 0 | 0 |
| 13 | 0 | .12 | 0 | 0 | 0 | 0 | 0 | 0 | 0 | 0 | 0 | 0 | 0 | 0 | 0 | 0 | 1 | 0 | 0 | 0 | 0 |
| 14 | 0 | 0 | 0 | 0 | 0 | 0 | 0 | 0 | 0 | .12 | .12 | 0 | 0 | 0 | 0 | 0 | 0 | 0 | 0 | .12 | 0 |
| 15 | 0 | 0 | 0 | 0 | 0 | 0 | 0 | 0 | 0 | .12 | 0 | 0 | 0 | 0 | 0 | 0 | 0 | 0 | 0 | 1 | 0 |
| 16 | 0 | 0 | 0 | 0 | 0 | 0 | 0 | 0 | 0 | 0 | .12 | 0 | .12 | 0 | 0 | 0 | 0 | 0 | 0 | 0 | 0 |
| 17 | 0 | 0 | 0 | 0 | 0 | 0 | 0 | 0 | 0 | 0 | 0 | 0 | 0 | .12 | 0 | 0 | 0 | 0 | 0 | 0 | 0 |
| 18 | 0 | 0 | 0 | 0 | 0 | 0 | 0 | 0 | 0 | 0 | 0 | 0 | 0 | 0 | .12 | 0 | 0 | 0 | 0 | 0 | 0 |
| 19 | 0 | 0 | 0 | 0 | 0 | .12 | 0 | 0 | 0 | 0 | .12 | 0 | 0 | 0 | 0 | 0 | 0 | 0 | 0 | .12 | 0 |
| 20 | 0 | 0 | 0 | 0 | 0 | 0 | 0 | 0 | 0 | 0 | 0 | 0 | 0 | 0 | .12 | 0 | 0 | 0 | 0 | 0 | 0 |
| 21 | 0 | 0 | 0 | 0 | 0 | 0 | 0 | 0 | 0 | 0 | 0 | 0 | 0 | .12 | 0 | 0 | 0 | 0 | 0 | 0 | 0 |
| 22 | .12 | 0 | 0 | 0 | 0 | .12 | 0 | 0 | .12 | 0 | .12 | 0 | 0 | 0 | .12 | 0 | 0 | 0 | 0 | 0 | 0 |
| 23 | 0 | 0 | 0 | 0 | 0 | 0 | 0 | .12 | 0 | 0 | 0 | 0 | 0 | 0 | 0 | 0 | 0 | 0 | 0 | 0 | 0 |
| 24 | 0 | 0 | 0 | 0 | 0 | 0 | 0 | 0 | .12 | 0 | 0 | 0 | 0 | 0 | 0 | 0 | 0 | 0 | 0 | 0 | 0 |
| 25 | .12 | 0 | 0 | 0 | 0 | .12 | 0 | 0 | .12 | .12 | .12 | 0 | .12 | 0 | 0 | .12 | 0 | 1 | 0 | .12 | 0 |
| 26 | 0 | 0 | .12 | 0 | 0 | 0 | 0 | 0 | 0 | 0 | 0 | 0 | 0 | 0 | 0 | 0 | 0 | 0 | 0 | 0 | 0 |
| 27 | 0 | 0 | 0 | 0 | 0 | 0 | 0 | 0 | 0 | 0 | 0 | 0 | 0 | 0 | .12 | 0 | 0 | 0 | 0 | 0 | 0 |
| 28 | 0 | 0 | 0 | 0 | 0 | 0 | 0 | 0 | 0 | .12 | 0 | 0 | .12 | 0 | 0 | 0 | 0 | 0 | 0 | 0 | 0 |
| 29 | 0 | 0 | 0 | 0 | 0 | 0 | 0 | 0 | 0 | 0 | .12 | 0 | .12 | 0 | 0 | 0 | 0 | 0 | 0 | 0 | 0 |
| 30 | 0 | 0 | 0 | 0 | 0 | .12 | 0 | 0 | 0 | 0 | 0 | 0 | 0 | 0 | 0 | 0 | 0 | 0 | 0 | 0 | 0 |
| 31 | 0 | 0 | 0 | 0 | 0 | 0 | 0 | 0 | 0 | 0 | .12 | 0 | 0 | 0 | 0 | 0 | 0 | 0 | 0 | 0 | 0 |
| 32 | .12 | 0 | 0 | 0 | 0 | 0 | 0 | 0 | 0 | 0 | 0 | 0 | 0 | 0 | 0 | 0 | 0 | 0 | 0 | 0 | 0 |
| 33 | 0 | 0 | .12 | 0 | 0 | 0 | 0 | 0 | 0 | 0 | 0 | 0 | 0 | 0 | 0 | 0 | 0 | 0 | 0 | 0 | 0 |
| 34 | 0 | 0 | 0 | 0 | 0 | 0 | 0 | 0 | 0 | .12 | 0 | 0 | 0 | 0 | 0 | 0 | 0 | 0 | 0 | .12 | 0 |
| 35 | 0 | 0 | 0 | 0 | 0 | 0 | 0 | 0 | 0 | 0 | .12 | 0 | 0 | 0 | 0 | 0 | 0 | 0 | 0 | 0 | 0 |
| 36 | 0 | 0 | 0 | 0 | 0 | 0 | 0 | 0 | 0 | 0 | 0 | 0 | 0 | 0 | 0 | 0 | 0 | 0 | .12 | 0 | 0 |
| 37 | 0 | .12 | 0 | 0 | 0 | 0 | 0 | 0 | 0 | 0 | 0 | 0 | 0 | 0 | 0 | .12 | 0 | 0 | 0 | 0 | 0 |
| 38 | 0 | .12 | 0 | 0 | 0 | .12 | 0 | 0 | 0 | 0 | 0 | .12 | 0 | 0 | 0 | 0 | 0 | 0 | 0 | 0 | 0 |
| 39 | 0 | 0 | 0 | 0 | 0 | 0 | 0 | .12 | 0 | 0 | 0 | 0 | 0 | 0 | 0 | 0 | 0 | 0 | 0 | 0 | 0 |
| 40 | 0 | 0 | 0 | 0 | 0 | 0 | 0 | 0 | 0 | 0 | 0 | 0 | 0 | .12 | 0 | 0 | 0 | 0 | 0 | 0 | 0 |
| 41 | .12 | 1 | 0 | 0 | 0 | 0 | .12 | 0 | 0 | 0 | 0 | 0 | 0 | 0 | 0 | .12 | 0 | 0 | 0 | 0 | 0 |
| 42 | 0 | 0 | 0 | 0 | 0 | 0 | 0 | 0 | 0 | .12 | 0 | 0 | 0 | 0 | 0 | 0 | 0 | 0 | 0 | 0 | 0 |
| 43 | 0 | 0 | 0 | 0 | 0 | 0 | 0 | 0 | 0 | 1 | 0 | 0 | 0 | 0 | 0 | 0 | .12 | 0 | 0 | 0 | 0 |
| 44 | 0 | 0 | 0 | 0 | 0 | 0 | 0 | 0 | 0 | .12 | 0 | 0 | 0 | 0 | 0 | 0 | 0 | 0 | 0 | .12 | 0 |
| 45 | 0 | 0 | 0 | 0 | 0 | 0 | 0 | 0 | 0 | 0 | .12 | 0 | 0 | .12 | 0 | 0 | 0 | 0 | 0 | 0 | 0 |
| 46 | 0 | 0 | 0 | 0 | 0 | 0 | 0 | 0 | .12 | 0 | 0 | 0 | 0 | 0 | 0 | .12 | 0 | 0 | 0 | 0 | 0 |
| 47 | 0 | 0 | 0 | 0 | 0 | 0 | 0 | 0 | 0 | 0 | 0 | 0 | 0 | 0 | 0 | 0 | .12 | 0 | 0 | 0 | 0 |
| 48 | 0 | 1 | .12 | 0 | 0 | 0 | 0 | 0 | 0 | 0 | 0 | 1 | 0 | 0 | 0 | 0 | 0 | 0 | 0 | 0 | 0 |
| 49 | .12 | 0 | 0 | 0 | 0 | 0 | 0 | 0 | 0 | 0 | 0 | 0 | 0 | .12 | 0 | 0 | 0 | 0 | 0 | 0 | 0 |
| 50 | 0 | 0 | .12 | 0 | 0 | 0 | 0 | 0 | 0 | 0 | 0 | 0 | 0 | 0 | 0 | 0 | 0 | 0 | 0 | 0 | 0 |
| 51 | .12 | 1 | 0 | 0 | 0 | 0 | 0 | 0 | 0 | 0 | 1 | .12 | 0 | 0 | 0 | 0 | 0 | 0 | 0 | 0 | 0 |
| 52 | 0 | 0 | 0 | 0 | 0 | 0 | 0 | 0 | 0 | 0 | 0 | 0 | 0 | 0 | .12 | 0 | 0 | 0 | 0 | 0 | 0 |
| 53 | 0 | .12 | 0 | 0 | .12 | .12 | .12 | 0 | 0 | 0 | .12 | .12 | 0 | 0 | 0 | 0 | 0 | 0 | 0 | .12 | 0 |
| 54 | .12 | 0 | 0 | 0 | 0 | 0 | 0 | 0 | 0 | 0 | 0 | 0 | 0 | 0 | .12 | 0 | .12 | 0 | 0 | .12 | 0 |
| 55 | 0 | 0 | 0 | 0 | 0 | 0 | 0 | .12 | 0 | 0 | 0 | 0 | 0 | 0 | 0 | .12 | .12 | .12 | 0 | .12 | 0 |
| 56 | .12 | 0 | .12 | .12 | 0 | 0 | 0 | 0 | .12 | 0 | .12 | 0 | 0 | 0 | 0 | 0 | 0 | 0 | 0 | 0 | 0 |
| 57 | 0 | 0 | 0 | 0 | 0 | 0 | 0 | 0 | 0 | 0 | 0 | 0 | 0 | 0 | 0 | 0 | 0 | 0 | .12 | 0 | 0 |
| 58 | 0 | 0 | 0 | 0 | 0 | 0 | 0 | 0 | 0 | 0 | 0 | 0 | 0 | 0 | .12 | 0 | 0 | 0 | 0 | 0 | 0 |
| 59 | 0 | 0 | 0 | 0 | 0 | .12 | 0 | 0 | .12 | 0 | 0 | 0 | 0 | 0 | 0 | 0 | 0 | 0 | 0 | 0 | 0 |
| 60 | 0 | 0 | 0 | 0 | 0 | 0 | 0 | 0 | 0 | 0 | 0 | 0 | 0 | .12 | 0 | 0 | 0 | 0 | 0 | 0 | 0 |
| 61 | 0 | 0 | 0 | .12 | 0 | .12 | .12 | 0 | 0 | 0 | 0 | 0 | 0 | 0 | 0 | .12 | .12 | 0 | 0 | 0 | 0 |
| 62 | 0 | 0 | 0 | 0 | 0 | 0 | 0 | 0 | 0 | 0 | 0 | 0 | 0 | .12 | 0 | 0 | 0 | 0 | 0 | 0 | 0 |
| 63 | 0 | .12 | 0 | 0 | 0 | 0 | 0 | 0 | 0 | 0 | 0 | 0 | 0 | 0 | 0 | 0 | 0 | 0 | 0 | 1 | 0 |
| 64 | 0 | 0 | 1 | 0 | 0 | 0 | 1 | 0 | 0 | 0 | .12 | 0 | .12 | .12 | .12 | .12 | 1 | 0 | 0 | 0 | 0 |
| 65 | 0 | 0 | 0 | 0 | 0 | 0 | 0 | 0 | 0 | .12 | 0 | 0 | 0 | 0 | 0 | 0 | 0 | 0 | 0 | .12 | 0 |
| 66 | 0 | 0 | 0 | 0 | 0 | 0 | 0 | 0 | 0 | 0 | .12 | 0 | 0 | 0 | 0 | 0 | 0 | 0 | 0 | 0 | 0 |
| 67 | 0 | 0 | 0 | .12 | 0 | 0 | 0 | 0 | 0 | 0 | 0 | 0 | 0 | 0 | 0 | 0 | 0 | 0 | 0 | 0 | 0 |
| 68 | 0 | 0 | .12 | 0 | 0 | 0 | 0 | 0 | 0 | 0 | 0 | .12 | 0 | 0 | 0 | 0 | 0 | 0 | 0 | 0 | 0 |
| 69 | .12 | 0 | .12 | .12 | 0 | .12 | 0 | 0 | 0 | 0 | 0 | 0 | .12 | 0 | 0 | .12 | .12 | 0 | 0 | 0 | 0 |
| 70 | 0 | 0 | 0 | 0 | 0 | 0 | 0 | 0 | 0 | 0 | 0 | .12 | 0 | 0 | .12 | .12 | 0 | 0 | 0 | 0 | 0 |
| 71 | 0 | .12 | 0 | 0 | 0 | .12 | 0 | .12 | 0 | 0 | .12 | 0 | 0 | 0 | 0 | 0 | .12 | 0 | 0 | .12 | 0 |
| 72 | 0 | 0 | 0 | .12 | 0 | 0 | .12 | 0 | 0 | 0 | 0 | 0 | 0 | 0 | .12 | .12 | .12 | 0 | 0 | 0 | 0 |
| 73 | 0 | 0 | 0 | 0 | 0 | 0 | .12 | 0 | 0 | 0 | 0 | 0 | 0 | 0 | .12 | .12 | .12 | 0 | 0 | 0 | 0 |
| 74 | 0 | .12 | 0 | 0 | 0 | 0 | 0 | 0 | .12 | 0 | 0 | 0 | 0 | 0 | 0 | 0 | 0 | .12 | 0 | 0 | 0 |
| 75 | 0 | 0 | 0 | 0 | 0 | 0 | 0 | .12 | 0 | .12 | .12 | 0 | .12 | 0 | 0 | 0 | 0 | 0 | 0 | 0 | 0 |
| 76 | 0 | .12 | 0 | 0 | 0 | 0 | 0 | 0 | 0 | 0 | 0 | 0 | 0 | 0 | 0 | .12 | .12 | 0 | 0 | 0 | 0 |
| 77 | 0 | 0 | .12 | 0 | 0 | 0 | 0 | 0 | 0 | 0 | 0 | 0 | 0 | 0 | 0 | 0 | 0 | 0 | 0 | 0 | 0 |
| 78 | 0 | .12 | 0 | 0 | 0 | .12 | .12 | .12 | 0 | .12 | .12 | .12 | .12 | .12 | .12 | 0 | 0 | 0 | 0 | .12 | 0 |
| 79 | 0 | 0 | 0 | 0 | 0 | 0 | 0 | .12 | 0 | 0 | 0 | 0 | 0 | 0 | .12 | .12 | 0 | 0 | 0 | .12 | 0 |
| 80 | .12 | 0 | 0 | .12 | 0 | .12 | 0 | 0 | 0 | 0 | .12 | 0 | 0 | 0 | .12 | 0 | .12 | 0 | 0 | 0 | 0 |
| 81 | .12 | 0 | 0 | 0 | .12 | 0 | 0 | 0 | .12 | 0 | .12 | 0 | .12 | 0 | 0 | .12 | .12 | 0 | 0 | .12 | 0 |
| 82 | 0 | 0 | 0 | 0 | 0 | 0 | 0 | .12 | 0 | 0 | 0 | 0 | 0 | 0 | 0 | 0 | 0 | 0 | 0 | 0 | 0 |
| 83 | .12 | 0 | .12 | .12 | 0 | 0 | 0 | 0 | 0 | 0 | 0 | .12 | 0 | 0 | .12 | .12 | .12 | 0 | 0 | 0 | 0 |
| 84 | .12 | 0 | .12 | .12 | 0 | 0 | 0 | .12 | .12 | 0 | .12 | 0 | 0 | 0 | 0 | 0 | 0 | 0 | 0 | .12 | 0 |
| 85 | .12 | 0 | 0 | 0 | 0 | 0 | .12 | 0 | 0 | 0 | .12 | 0 | 0 | 0 | 0 | 0 | .12 | 0 | 0 | .12 | 0 |
| 86 | 0 | 0 | 0 | 0 | 0 | 0 | 0 | .12 | 0 | 0 | 0 | 0 | 0 | 0 | 0 | 0 | 0 | 0 | 0 | 0 | 0 |
| 87 | .12 | .12 | 0 | 0 | 0 | .12 | .12 | 0 | .12 | 0 | .12 | .12 | .12 | 0 | 0 | 0 | 0 | .12 | 0 | 0 | 0 |
| 88 | .12 | .12 | 0 | 0 | .12 | 0 | 0 | 0 | .12 | .12 | .12 | .12 | .12 | 0 | 0 | 0 | .12 | 0 | 0 | .12 | 0 |
| 89 | 0 | 0 | 0 | 0 | 0 | 0 | 0 | 0 | 0 | .12 | 0 | 0 | 0 | 0 | 0 | 0 | 0 | 0 | 0 | 0 | 0 |
| 90 | 0 | 0 | 0 | 0 | 0 | .12 | 0 | 0 | 0 | 0 | 0 | 0 | 0 | 0 | .12 | .12 | .12 | 0 | 0 | 0 | 0 |
| 91 | 0 | .12 | .12 | 0 | 0 | .12 | 0 | 0 | 0 | .12 | .12 | 0 | 0 | 0 | 0 | 0 | .12 | 0 | 0 | .12 | 0 |
| 92 | 0 | 0 | 0 | 0 | 0 | 0 | 0 | 0 | 0 | .12 | .12 | 0 | 0 | 0 | 0 | 0 | 0 | 0 | 0 | 0 | 0 |
| 93 | 0 | 0 | .12 | 0 | 0 | 0 | 0 | 0 | 0 | 0 | 0 | 0 | 0 | 0 | 0 | 0 | 0 | 0 | 0 | 0 | 0 |
| 94 | 0 | .12 | 0 | 0 | .12 | 0 | .12 | 0 | 0 | 0 | 0 | .12 | 0 | 0 | 0 | 0 | 0 | 0 | 0 | 0 | 0 |
| 95 | 0 | 0 | .12 | 0 | 0 | 0 | 0 | 0 | .12 | 0 | .12 | 0 | 0 | 0 | 0 | 0 | 0 | .12 | 0 | 0 | 0 |
| 96 | 0 | 0 | 0 | 0 | .12 | 0 | 0 | .12 | 0 | 0 | 0 | .12 | 0 | 0 | 0 | 0 | 0 | 0 | 0 | 0 | 0 |
| 97 | .12 | 0 | 0 | 0 | 0 | 0 | 0 | 0 | 0 | 0 | 0 | 0 | 0 | 0 | 0 | .12 | 0 | 0 | 0 | .12 | 0 |
| 98 | 1 | 0 | 0 | .12 | 0 | 0 | .12 | 0 | 0 | 0 | 0 | 0 | 0 | 0 | .12 | .12 | .12 | 0 | 0 | 0 | 0 |
| 99 | 0 | 1 | 0 | 0 | 0 | 1 | .12 | 0 | 0 | 0 | .12 | 1 | 0 | 0 | .12 | .12 | 0 | 0 | 0 | .12 | 0 |
| 100 | 0 | 0 | 0 | 0 | 0 | 0 | 0 | 0 | 0 | 0 | .12 | 0 | 0 | .12 | 0 | 0 | 0 | 0 | 0 | 0 | 0 |
| 101 | 0 | .12 | 0 | 0 | 0 | 0 | 1 | 0 | 0 | 0 | 0 | 1 | 0 | 0 | 0 | 0 | 0 | 0 | 0 | 0 | 0 |
| 102 | 1 | .12 | 0 | 0 | 0 | 1 | 1 | 0 | 0 | 0 | 0 | .12 | 0 | 0 | 0 | 0 | 0 | 0 | 0 | 0 | 0 |
| 103 | 1 | 0 | 0 | 0 | 0 | 0 | .12 | 0 | 0 | 1 | 1 | 1 | 0 | 0 | 0 | 0 | 1 | 0 | 0 | 1 | 0 |
| 104 | 0 | 0 | 0 | 0 | 0 | 0 | 0 | 0 | 0 | 0 | .12 | 0 | 0 | 0 | 0 | 0 | 0 | 0 | 0 | 0 | 0 |
| 105 | .12 | 0 | 0 | 0 | 0 | 0 | .12 | 0 | 0 | 0 | 0 | 1 | 0 | 0 | 0 | 0 | 0 | 0 | 0 | 0 | 0 |
| 106 | 1 | 1 | 1 | .12 | 0 | 1 | 1 | .12 | 0 | .12 | .12 | 1 | 0 | 0 | 0 | .12 | .12 | 0 | .12 | 0 | 0 |
| 107 | .12 | 0 | 0 | 0 | .12 | 0 | 0 | 0 | 0 | 1 | .12 | 0 | 0 | 0 | 0 | 0 | .12 | 0 | 1 | .12 | 0 |
| 108 | 0 | 0 | 0 | 0 | .12 | 0 | 0 | 0 | .12 | 1 | 1 | 0 | .12 | 0 | 0 | 0 | 0 | 0 | 0 | 0 | 0 |
| 109 | 1 | .12 | 1 | 0 | 0 | 0 | 1 | 0 | 0 | 0 | .12 | 0 | 0 | 0 | 0 | .12 | 0 | .12 | 0 | 0 | 0 |
| 110 | .12 | 0 | 0 | .12 | 0 | 0 | 0 | .12 | 0 | 0 | 0 | 0 | 0 | 0 | 0 | .12 | 0 | 0 | 0 | 0 | 0 |
| 111 | 0 | 0 | 0 | 0 | .12 | 0 | 0 | .12 | 0 | 0 | 0 | 0 | 0 | 0 | 0 | .12 | .12 | 0 | 0 | 0 | 0 |
| 112 | .12 | 0 | .12 | 0 | 0 | 0 | .12 | 0 | 0 | 0 | 0 | 0 | 0 | 0 | .12 | .12 | 0 | 0 | 0 | 0 | 0 |
| 113 | 0 | 0 | 0 | .12 | 0 | 0 | .12 | .12 | 0 | 0 | 0 | 0 | 0 | 0 | .12 | .12 | 0 | 0 | 0 | 0 | 0 |
| 114 | 0 | 0 | 0 | .12 | 0 | 0 | 0 | 0 | 0 | 0 | 0 | 0 | 0 | 0 | .12 | 0 | 0 | 0 | 0 | 0 | 0 |
| 115 | 0 | .12 | 0 | 0 | 0 | 0 | .12 | 0 | 0 | 0 | 0 | 0 | 0 | 0 | 0 | 0 | 0 | 0 | 0 | 0 | 0 |
| 116 | 0 | .12 | 0 | .12 | 0 | 0 | .12 | 0 | 0 | 0 | 0 | 0 | 0 | 0 | 0 | .12 | 1 | 0 | 0 | 0 | 0 |
| 117 | 0 | 0 | 0 | 0 | 0 | 0 | 0 | 0 | 0 | 1 | 0 | 0 | 0 | 0 | 0 | 0 | 0 | 0 | 0 | .12 | 0 |
| 118 | .12 | 1 | 0 | 0 | .12 | 0 | 0 | 0 | 0 | 0 | 0 | 0 | 0 | 0 | 0 | .12 | .12 | 0 | 0 | .12 | 0 |
| 119 | 0 | 0 | 0 | 0 | .12 | 0 | 0 | 0 | 0 | 0 | 0 | 0 | 0 | 0 | 0 | 0 | 0 | 0 | 0 | 0 | 0 |
| 120 | 0 | 0 | 0 | 0 | 0 | 0 | 0 | 0 | 0 | 0 | .12 | 0 | 0 | 0 | 0 | 0 | 0 | 0 | 0 | .12 | 0 |
| 121 | 0 | 0 | 0 | 0 | 0 | 0 | 0 | 0 | 0 | .12 | 0 | 0 | 0 | 0 | 0 | 0 | 0 | 0 | 0 | 0 | 0 |
| 122 | 0 | 0 | 0 | 0 | 0 | 0 | 0 | 0 | 0 | 0 | 0 | 0 | 0 | 0 | 0 | 0 | .12 | 0 | 0 | 0 | 0 |
| 123 | 0 | 0 | 0 | .12 | 0 | 0 | 0 | 0 | 0 | 0 | 0 | 0 | 0 | 0 | 0 | 0 | 0 | 0 | 0 | 0 | 0 |
| 124 | .12 | 0 | 0 | 0 | 0 | 0 | 0 | 0 | 0 | 0 | 0 | 0 | 0 | 0 | .12 | 0 | 0 | 0 | 0 | 0 | 0 |
| 125 | 0 | 0 | 0 | 0 | 0 | 0 | 0 | 0 | 0 | 0 | .12 | 0 | .12 | 0 | 0 | 0 | 0 | 0 | 0 | 0 | 0 |
| 126 | 0 | 0 | 0 | 0 | 0 | 0 | 0 | 0 | 0 | 0 | 0 | 0 | 0 | 0 | 0 | 0 | 0 | .12 | 0 | 0 | 0 |
| 127 | 0 | 0 | 0 | 0 | 0 | 0 | 0 | 0 | 0 | 0 | 0 | 0 | 0 | 0 | 0 | 0 | 0 | 0 | .12 | 0 | 0 |
| 128 | 0 | 0 | 0 | 0 | 0 | 0 | 0 | 0 | 0 | 0 | 0 | 0 | 0 | .12 | 0 | 0 | 0 | 0 | 0 | 0 | 0 |
| 129 | .12 | 0 | 0 | 0 | 0 | 0 | 0 | 0 | 0 | 0 | 0 | 0 | 0 | 0 | .12 | 0 | 1 | 0 | 0 | 0 | 0 |
| 130 | 0 | 0 | 0 | 0 | 0 | .12 | 1 | 0 | 0 | 0 | 1 | 0 | 0 | 0 | 0 | 0 | 0 | 0 | 0 | 0 | 0 |
| 131 | .12 | 0 | 0 | 1 | 0 | 0 | 1 | 0 | 0 | 0 | 0 | 0 | 0 | 0 | .12 | .12 | 0 | 0 | 0 | .12 | 0 |
| 132 | 0 | 0 | 0 | 0 | 0 | 0 | 0 | 0 | 0 | 0 | 0 | 0 | 0 | 0 | 0 | 0 | 0 | 0 | 0 | .12 | 0 |
| 133 | .12 | 0 | 0 | 0 | 0 | 0 | 0 | 0 | 0 | 0 | 0 | 0 | 0 | 0 | 0 | 0 | 0 | 0 | 0 | 0 | 0 |
| 134 | 0 | 0 | 0 | .12 | 0 | 0 | .12 | .12 | 0 | 0 | 0 | 1 | 0 | 0 | 0 | .12 | 0 | 0 | 0 | 0 | 0 |
| 135 | .12 | 1 | 0 | 0 | 0 | 0 | 1 | 0 | 0 | 0 | 0 | 1 | 0 | 0 | 0 | .12 | 0 | 0 | 0 | 0 | 0 |
| 136 | 0 | 0 | 0 | 0 | 0 | 0 | 0 | 0 | 0 | 0 | .12 | 0 | 0 | 0 | 0 | 0 | 0 | 0 | 0 | 0 | 0 |
| 137 | 0 | 0 | .12 | 0 | 0 | 0 | 0 | .12 | 0 | 0 | 0 | .12 | 0 | 0 | 0 | 0 | 0 | 0 | 0 | 0 | 0 |
| 138 | 0 | 0 | 0 | 0 | 0 | 0 | 0 | 0 | 0 | .12 | .12 | 0 | 0 | 0 | 0 | 0 | 0 | 0 | 0 | .12 | 0 |
| 139 | .12 | .12 | .12 | 0 | 0 | 0 | 0 | 0 | 0 | 0 | 0 | .12 | 0 | 0 | .12 | .12 | 0 | 0 | 0 | 0 | 0 |
| 140 | 0 | .12 | 0 | 0 | 0 | 0 | 0 | 0 | 0 | 0 | 1 | 0 | 0 | 0 | 0 | 0 | 0 | 0 | 0 | 0 | 0 |
| 141 | 0 | 0 | 0 | 0 | 0 | 0 | 0 | 0 | 0 | 0 | .12 | 0 | .12 | 0 | 0 | 0 | 0 | 0 | 0 | 0 | 0 |
| 142 | 0 | 0 | 0 | 0 | 0 | 0 | 0 | 0 | 0 | 0 | 0 | 0 | 0 | 0 | 0 | 0 | 0 | 0 | 0 | .12 | 0 |
| 143 | 0 | 0 | 0 | 0 | 0 | 0 | 0 | 0 | 0 | 0 | .12 | 0 | 0 | 0 | 0 | 0 | 0 | 0 | 0 | 0 | 0 |
| 144 | 0 | 0 | 0 | 0 | 0 | .12 | 0 | 0 | 0 | 0 | 0 | 0 | .12 | 0 | 0 | 0 | 0 | 0 | 0 | 0 | 0 |
| 145 | 0 | 0 | 0 | 0 | 0 | 0 | 0 | 0 | 0 | 0 | 0 | 0 | 0 | 0 | 0 | 0 | .12 | 0 | 0 | 0 | 0 |
| 146 | 0 | 0 | 0 | 0 | 0 | 0 | 0 | .12 | 0 | 0 | 0 | 0 | 0 | 0 | 0 | .12 | 0 | 0 | 0 | 0 | 0 |
| 147 | 0 | 0 | 0 | 0 | 0 | 0 | 0 | 0 | 0 | 0 | 0 | 0 | 0 | 0 | 0 | .12 | 0 | 0 | 0 | 0 | 0 |
| 148 | 0 | 0 | 0 | 0 | 0 | 0 | 0 | 0 | 0 | 0 | .12 | 0 | 0 | 0 | 0 | 0 | 0 | 0 | 0 | 0 | 0 |
| 149 | 0 | 0 | 0 | 0 | 0 | 0 | 0 | 0 | 0 | 0 | 0 | 0 | 0 | .12 | 0 | 0 | 0 | 0 | 0 | 0 | 0 |
| 150 | 0 | 0 | .12 | 0 | 0 | 0 | 0 | 0 | 0 | 0 | 0 | 0 | 0 | 0 | 0 | 0 | 0 | 0 | 0 | 0 | 0 |
| 151 | 0 | 0 | 0 | 0 | 0 | 0 | 0 | 0 | 0 | 0 | 0 | 0 | 0 | .12 | 0 | 0 | 0 | 0 | 0 | 0 | 0 |
| 152 | 0 | 0 | 0 | 0 | 0 | 0 | 0 | 0 | .12 | 0 | 0 | 0 | 0 | 0 | 0 | 0 | 0 | 0 | 0 | 0 | 0 |
| 153 | .12 | 0 | 0 | 0 | .12 | 0 | 0 | 0 | 0 | 0 | 0 | 0 | 0 | 0 | 0 | .12 | .12 | 0 | 0 | .12 | 0 |
| 154 | .12 | 0 | 0 | 0 | 0 | .12 | 0 | 0 | .12 | 0 | .12 | 0 | 0 | 0 | 0 | 0 | 0 | 0 | 0 | .12 | 0 |
| 155 | 0 | 0 | 0 | 0 | 0 | 0 | 0 | 0 | 0 | 0 | .12 | 0 | 0 | 0 | 0 | 0 | 0 | 0 | 0 | .12 | 0 |
| 156 | .12 | 0 | 0 | 0 | .12 | 0 | 0 | 0 | 0 | 0 | 0 | 0 | 0 | 0 | 0 | .12 | 0 | 0 | 0 | 0 | 0 |
| 157 | 0 | 0 | 0 | 0 | 0 | .12 | 0 | .12 | 0 | 0 | .12 | 0 | 0 | 0 | 0 | 0 | 0 | 0 | 0 | 0 | 0 |
| 158 | 0 | 0 | .12 | .12 | 0 | .12 | .12 | 0 | 0 | 0 | 0 | 0 | 0 | 0 | .12 | 0 | .12 | 0 | 0 | 0 | 0 |
| 159 | 0 | .12 | .12 | 0 | 0 | .12 | 1 | 0 | 0 | 0 | .12 | .12 | .12 | 0 | 0 | 0 | 0 | 0 | 0 | 0 | 0 |
| 160 | 0 | 0 | 0 | 0 | 0 | 0 | 0 | 0 | 0 | 0 | 0 | 0 | 0 | .12 | 0 | 0 | 0 | 0 | 0 | 0 | 0 |
| 161 | 0 | 0 | 0 | .12 | 0 | 0 | .12 | 0 | 0 | 1 | .12 | 0 | 0 | 0 | 0 | 0 | 0 | 0 | 0 | 0 | 0 |
| 162 | .12 | 0 | 0 | 0 | 0 | 1 | .12 | 0 | 0 | 0 | 0 | 1 | 0 | 0 | 0 | 0 | 0 | 0 | 0 | 0 | 0 |
| 163 | .12 | .12 | 0 | 0 | 0 | 1 | 0 | 0 | 0 | 0 | .12 | .12 | 0 | 0 | 0 | 0 | 0 | 0 | 0 | 0 | 0 |
| 164 | 0 | 0 | 0 | 0 | 0 | 0 | 0 | .12 | 0 | 0 | 0 | 0 | 0 | 0 | 0 | 0 | 0 | 0 | 0 | 0 | 0 |
| 165 | 0 | 0 | 0 | 0 | 0 | 0 | 0 | 0 | 0 | 0 | .12 | 0 | 0 | 0 | 0 | 0 | 0 | 0 | .12 | 0 | 0 |
| 166 | 0 | 0 | 0 | 0 | 0 | 0 | 0 | 0 | 0 | 0 | .12 | 0 | 0 | 0 | 0 | 0 | 0 | 0 | 0 | 0 | 0 |
| 167 | 0 | 0 | 1 | .12 | 0 | 0 | .12 | 0 | 0 | 0 | 0 | 0 | 0 | 0 | 0 | 0 | 0 | 0 | 0 | 0 | 0 |
| 168 | 0 | 0 | 0 | 0 | .12 | 0 | 0 | 0 | 0 | 0 | 0 | 0 | 0 | 0 | .12 | 0 | .12 | 0 | 0 | 0 | 0 |
| 169 | 1 | 0 | 1 | .12 | .12 | .12 | .12 | .12 | 0 | 0 | .12 | 0 | 0 | 0 | 0 | .12 | .12 | 0 | 0 | .12 | 0 |
| 170 | .12 | 0 | 1 | .12 | .12 | 0 | 0 | .12 | .12 | 0 | 0 | 0 | 0 | 0 | .12 | .12 | .12 | 0 | 0 | .12 | 0 |
| 171 | .12 | 0 | 1 | .12 | 0 | .12 | .12 | 0 | 0 | 0 | .12 | .12 | 0 | 0 | .12 | .12 | .12 | 0 | 0 | .12 | 0 |
| 172 | 0 | 0 | .12 | 0 | 0 | 0 | 1 | 0 | .12 | 0 | 0 | 1 | 0 | 0 | 0 | .12 | .12 | 0 | 0 | 0 | 0 |
| 173 | 0 | .12 | .12 | 1 | 0 | 0 | 0 | .12 | 0 | .12 | .12 | .12 | 0 | 0 | .12 | .12 | 0 | 0 | 0 | 0 | 0 |
| 174 | 0 | 0 | 0 | 0 | 0 | 0 | .12 | 0 | 0 | 0 | .12 | 0 | .12 | 0 | .12 | .12 | 0 | 0 | 0 | 0 | 0 |
| 175 | 0 | 0 | .12 | .12 | 0 | 0 | .12 | 0 | 0 | 0 | 0 | 0 | 0 | 0 | 0 | 0 | 0 | 0 | 0 | 0 | 0 |
| 176 | 1 | 0 | 0 | 0 | 0 | 0 | .12 | 0 | 0 | 0 | 0 | 1 | 0 | 0 | 0 | 0 | 0 | 0 | 0 | 0 | 0 |
| 177 | 0 | 0 | 0 | 0 | 0 | .12 | 0 | 0 | 0 | 0 | 0 | .12 | 0 | 0 | .12 | .12 | 0 | 0 | 0 | 0 | 0 |
| 178 | .12 | 0 | 0 | 0 | 0 | 0 | 0 | 0 | 0 | 0 | 0 | 0 | 0 | 0 | 0 | 0 | 0 | 0 | 0 | .12 | 0 |
| 179 | .12 | 0 | 0 | 0 | 0 | 0 | 1 | 0 | 0 | 0 | .12 | 0 | 0 | 0 | 1 | .12 | 0 | 0 | 0 | .12 | 0 |
| 180 | 0 | 0 | 0 | 0 | 0 | 0 | 0 | .12 | 0 | 0 | 0 | 0 | 0 | 0 | 0 | 0 | 0 | 0 | 0 | 0 | 0 |
| 181 | 0 | 0 | 0 | 0 | 0 | 0 | 0 | 0 | 0 | 0 | 0 | 0 | 0 | .12 | 0 | 0 | 0 | 0 | 0 | 0 | 0 |
| 182 | 0 | 0 | 0 | 0 | 0 | 0 | 0 | 0 | 0 | 0 | 0 | 0 | 0 | 0 | .12 | 0 | 0 | 0 | 0 | 0 | 0 |
| 183 | 0 | 0 | 0 | 0 | 0 | 0 | 1 | 0 | 0 | .12 | .12 | 0 | .12 | 0 | .12 | 0 | 0 | 1 | 1 | 0 | 0 |
| 184 | 0 | 0 | 0 | 0 | 0 | 0 | 0 | 0 | 0 | 0 | .12 | 0 | 0 | 0 | 0 | 0 | 0 | 0 | 0 | 0 | 0 |
| 185 | 0 | .12 | 0 | 0 | 0 | 0 | 0 | 0 | 0 | 0 | 0 | .12 | 0 | 0 | 0 | 0 | 0 | 0 | 0 | 0 | 0 |
| 186 | 0 | 1 | 1 | 0 | 0 | 0 | 0 | 0 | 0 | 0 | 0 | 0 | 0 | 0 | 0 | 0 | 0 | 0 | 0 | .12 | 0 |
| 187 | 0 | 1 | 0 | 0 | 0 | 0 | 0 | 0 | 0 | 0 | 0 | .12 | 0 | 0 | 0 | 0 | 0 | 0 | 0 | 0 | 0 |
| 188 | 0 | 0 | 0 | .12 | 0 | 0 | 0 | 0 | 0 | 0 | 0 | 0 | 0 | 0 | 0 | 0 | 0 | 0 | 0 | 0 | 0 |
| 189 | 0 | 0 | 0 | 0 | 0 | 0 | 0 | 0 | 0 | .12 | .12 | 0 | 0 | 0 | 0 | 0 | 0 | 0 | 0 | 0 | 0 |
| 190 | 0 | .12 | 0 | 0 | 0 | 0 | 0 | 0 | 0 | 0 | 0 | .12 | 0 | 0 | 0 | 0 | 0 | 0 | 0 | 0 | 0 |
| 191 | 0 | .12 | 0 | 0 | 0 | 0 | 0 | 0 | 0 | 0 | 0 | 0 | 0 | 0 | 0 | .12 | 0 | 0 | 0 | 0 | 0 |
| 192 | .12 | 0 | 0 | 0 | 0 | 0 | 0 | 0 | 0 | 0 | 0 | 0 | 0 | 0 | 0 | 0 | 0 | 0 | 0 | 0 | 0 |
| 193 | 0 | 0 | 0 | 0 | 0 | 0 | 0 | 0 | 0 | 0 | 0 | 0 | 0 | .12 | 0 | 0 | 0 | 0 | .12 | 0 | 0 |
| 194 | 0 | 0 | 0 | 0 | 0 | .12 | 0 | 0 | 0 | 0 | 0 | 0 | 0 | 0 | .12 | .12 | 0 | 0 | 0 | 0 | 0 |
| 195 | 0 | 0 | .12 | 0 | 0 | .12 | 0 | .12 | .12 | 0 | 0 | 0 | 0 | 0 | 0 | 0 | 0 | 0 | 0 | 0 | 0 |
| 196 | 0 | 0 | 0 | 0 | 0 | 0 | 0 | 0 | 0 | .12 | 0 | 0 | 0 | 0 | 0 | 0 | 0 | .12 | 0 | 0 | 0 |
| 197 | 0 | .12 | 0 | 0 | 0 | .12 | 0 | 0 | 0 | 0 | 0 | .12 | 0 | 0 | 0 | 0 | 0 | 0 | 0 | 0 | 0 |
| 198 | 0 | 0 | 0 | .12 | 0 | 0 | .12 | 0 | 0 | .12 | 0 | 0 | .12 | 0 | 0 | .12 | .12 | 0 | 0 | .12 | 0 |
| 199 | .12 | 0 | 0 | .12 | 0 | .12 | .12 | .12 | .12 | 0 | .12 | .12 | .12 | 0 | 0 | .12 | 0 | 0 | .12 | 0 | 0 |
| 200 | 0 | .12 | .12 | 0 | 0 | 0 | 0 | 0 | 0 | 0 | 0 | .12 | 0 | 0 | 0 | .12 | 0 | 0 | 0 | 0 | 0 |
| 201 | 0 | 0 | 0 | 0 | 0 | 0 | .12 | 0 | 0 | 0 | 0 | 0 | 0 | 0 | .12 | 0 | .12 | 0 | 0 | 0 | 0 |
| 202 | 0 | 0 | 0 | 0 | 0 | .12 | 0 | 0 | .12 | .12 | 0 | 0 | 0 | 0 | 0 | 0 | .12 | 0 | 0 | .12 | 0 |
| 203 | 0 | 0 | 0 | 0 | 0 | 0 | 0 | 0 | 0 | 0 | .12 | 0 | 0 | 0 | 0 | 0 | 0 | 0 | 0 | 0 | 0 |
| 204 | 0 | 0 | 0 | .12 | 0 | 0 | 1 | .12 | 0 | 0 | 0 | 0 | .12 | 0 | 0 | 0 | .12 | 0 | 0 | 0 | 0 |
| 205 | 0 | 0 | 0 | 0 | 0 | 0 | 0 | 0 | 0 | 0 | 0 | .12 | 0 | 0 | .12 | 0 | 0 | 0 | 0 | 0 | 0 |
| 206 | 0 | 0 | 0 | 0 | 0 | 0 | 0 | 0 | 0 | 0 | 0 | .12 | .12 | 0 | 0 | 0 | 0 | 0 | 0 | 0 | 0 |
| 207 | 0 | 0 | 0 | 0 | 0 | 0 | 0 | 0 | 0 | .12 | .12 | 0 | 0 | 0 | 0 | 0 | 0 | 0 | 0 | .12 | 0 |
| 208 | .12 | 0 | .12 | .12 | 0 | 0 | .12 | .12 | 0 | .12 | .12 | .12 | 0 | 0 | 0 | 0 | .12 | .12 | 0 | .12 | 0 |
| 209 | .12 | 0 | 0 | 0 | 0 | .12 | .12 | .12 | 0 | 0 | 0 | 0 | 0 | 0 | 0 | 0 | .12 | 0 | .12 | 0 | 0 |
| 210 | 0 | 0 | 0 | 0 | 0 | 0 | 0 | 0 | 0 | 0 | 0 | 0 | 0 | 0 | 0 | 0 | .12 | 0 | 0 | 0 | 0 |
| 211 | 0 | .12 | 0 | 0 | 0 | 0 | 0 | 0 | 0 | 0 | 0 | 1 | 0 | 0 | 0 | 0 | 0 | 0 | 0 | 0 | 0 |
| 212 | .12 | 0 | 0 | 0 | 0 | 0 | 1 | 0 | 0 | 0 | .12 | 0 | 0 | 0 | 0 | 0 | 0 | 0 | 0 | 0 | 0 |
| 213 | 0 | 0 | 0 | 0 | 0 | 0 | 0 | 0 | 0 | 0 | 0 | 0 | 0 | 0 | 0 | .12 | 0 | 0 | 0 | 0 | 0 |
| 214 | 0 | .12 | 0 | 0 | 0 | 0 | 0 | 0 | 0 | 0 | .12 | .12 | 0 | 0 | 0 | .12 | 0 | 0 | 0 | 0 | 0 |
| 215 | 0 | 0 | 0 | 0 | 0 | 0 | 0 | .12 | 0 | 0 | 0 | 0 | 0 | 0 | 0 | 0 | 0 | 0 | 0 | 0 | 0 |
| 216 | 0 | 0 | 0 | 0 | 0 | 0 | 0 | 0 | 0 | 0 | 0 | 0 | 0 | 0 | 0 | 0 | 0 | 0 | 0 | .12 | 0 |
| 217 | 0 | 0 | 0 | 0 | 0 | 0 | 0 | 0 | 0 | .12 | 0 | 0 | 0 | 0 | 0 | 0 | 0 | 0 | 0 | 0 | 0 |
| 218 | 0 | 0 | 0 | 0 | 0 | 0 | 0 | 0 | 0 | 0 | 0 | 0 | 0 | 0 | 0 | 0 | 0 | .12 | 0 | 0 | 0 |
| 219 | 0 | 0 | .12 | 0 | 0 | 0 | 0 | 0 | 0 | 0 | 0 | 0 | 0 | 0 | 0 | 0 | 0 | 0 | 0 | 0 | 0 |
| 220 | 0 | 0 | 0 | 0 | 0 | 0 | 0 | 0 | 0 | 0 | 0 | 0 | 0 | 0 | 0 | .12 | 0 | 0 | 0 | 0 | 0 |
| 221 | .12 | 0 | 0 | 0 | 0 | 0 | 0 | 0 | 0 | 0 | 0 | 0 | 0 | .12 | 0 | 0 | 0 | 0 | 0 | 0 | 0 |
| 222 | .12 | .12 | .12 | 0 | 0 | .12 | 0 | 0 | .12 | 0 | 0 | .12 | 0 | 0 | .12 | 0 | 0 | 0 | 0 | 0 | 0 |
| 223 | 0 | 0 | 0 | 0 | 0 | 0 | .12 | 0 | 0 | 0 | 0 | 0 | 0 | 0 | 0 | 0 | 0 | 0 | 0 | 0 | 0 |
| 224 | 0 | 0 | 0 | 0 | 0 | 0 | 0 | 0 | 0 | 0 | .12 | 0 | 0 | 0 | 0 | 0 | 0 | 0 | 0 | 0 | 0 |
| 225 | 0 | 0 | 0 | 0 | 0 | 0 | .12 | 0 | 0 | 0 | 0 | 0 | 0 | 0 | 0 | 0 | 0 | 0 | 0 | 0 | 0 |
| 226 | 0 | .12 | 0 | 0 | 0 | 0 | .12 | 0 | 0 | 0 | 0 | 0 | 0 | 0 | 0 | .12 | .12 | 0 | 0 | 0 | 0 |
| 227 | 0 | 0 | 0 | 0 | 0 | 0 | 0 | 0 | 0 | 0 | 0 | 0 | 0 | 0 | 0 | .12 | .12 | 0 | 0 | 0 | 0 |
| 228 | 0 | 0 | 0 | 0 | 0 | 0 | .12 | 0 | 0 | 0 | 0 | 0 | 0 | 0 | 0 | 0 | 0 | 0 | 0 | 0 | 0 |
| 229 | 0 | 0 | 0 | 0 | 0 | .12 | 0 | 0 | 0 | 0 | .12 | 0 | .12 | 0 | 0 | 0 | 0 | 0 | 0 | 0 | 0 |
| 230 | 0 | 0 | 0 | 0 | 0 | 0 | .12 | 0 | 0 | 0 | 0 | 0 | 0 | 0 | 0 | .12 | 0 | 0 | 0 | 0 | 0 |
| 231 | 0 | 0 | 0 | 0 | 0 | 0 | 0 | 0 | 0 | 0 | 0 | 0 | 0 | 0 | 0 | 0 | .12 | 0 | 0 | 0 | 0 |
| 232 | 0 | 0 | 0 | 0 | 0 | 0 | 0 | 0 | 0 | .12 | 0 | 0 | 0 | 0 | 0 | 0 | 0 | 0 | 0 | .12 | 0 |
| 233 | .12 | .12 | 0 | 0 | .12 | .12 | 0 | .12 | 0 | .12 | .12 | .12 | .12 | 0 | 0 | .12 | .12 | .12 | 0 | .12 | 0 |
| 234 | .12 | .12 | .12 | 0 | 0 | .12 | .12 | 0 | .12 | 0 | 0 | 0 | 0 | 0 | 0 | .12 | .12 | 0 | 0 | 0 | 0 |
| 235 | 0 | 0 | 0 | 0 | 0 | 0 | .12 | 0 | 0 | 0 | 0 | 0 | 0 | 0 | 0 | 0 | 0 | 0 | 0 | 0 | 0 |
| 236 | 0 | 0 | 0 | 0 | 0 | 0 | 0 | 0 | 0 | .12 | 0 | 0 | 0 | 0 | 0 | 0 | 0 | 0 | 0 | 0 | 0 |
| 237 | 0 | 0 | 0 | 0 | 0 | 0 | 0 | 0 | 0 | 0 | 0 | 0 | 0 | 0 | .12 | 0 | 0 | 0 | 0 | 0 | 0 |
| 238 | .12 | 0 | 0 | 0 | 0 | 0 | 0 | .12 | 0 | 0 | 0 | .12 | 0 | 0 | 0 | 0 | 0 | 0 | 0 | 0 | 0 |
| 239 | 0 | 0 | 0 | 0 | 0 | 0 | 0 | 0 | 0 | 0 | 0 | 0 | 0 | 0 | .12 | 0 | 0 | 0 | 0 | 0 | 0 |
| 240 | 0 | 0 | 0 | 0 | 0 | 0 | 0 | 0 | 0 | 0 | 0 | 0 | 0 | 0 | 0 | .12 | 0 | 0 | 0 | 0 | 0 |
| 241 | 0 | 0 | 0 | 0 | 0 | 0 | 0 | 0 | 0 | 0 | 0 | 0 | 0 | .12 | 0 | 0 | 0 | 0 | 0 | 0 | 0 |
| 242 | 0 | 0 | 0 | 0 | 0 | 0 | 0 | 0 | 0 | 0 | .12 | 0 | 0 | 0 | 0 | 0 | 0 | 0 | 0 | 0 | 0 |
| 243 | 0 | 0 | 0 | 0 | .12 | 0 | 0 | 0 | 0 | .12 | .12 | 0 | .12 | 0 | 0 | 0 | 0 | 0 | .12 | .12 | 0 |
| 244 | 0 | 0 | 0 | 0 | 0 | 0 | 0 | 0 | 0 | 0 | .12 | 0 | 0 | 0 | .12 | 0 | 0 | 0 | 0 | 0 | 0 |
| 245 | 0 | 0 | 0 | 0 | 0 | 0 | 0 | .12 | 0 | 0 | .12 | 0 | 0 | 0 | 0 | 0 | 0 | 0 | 0 | 0 | 0 |
| 246 | 0 | 0 | 0 | 0 | 0 | 0 | 0 | 0 | 0 | 0 | 0 | 0 | 0 | 0 | .12 | 0 | 0 | 0 | 0 | 0 | 0 |
| 247 | .12 | 0 | 0 | 0 | .12 | 0 | 0 | 0 | 0 | 0 | .12 | .12 | 0 | 0 | 0 | 0 | 0 | 0 | 0 | 0 | 0 |
| 248 | 0 | 0 | 0 | 0 | .12 | 0 | 0 | 0 | .12 | 0 | .12 | 0 | 0 | 0 | 0 | 0 | 0 | .12 | 0 | 0 | 0 |
| 249 | 0 | 0 | .12 | 0 | 0 | 0 | .12 | 0 | .12 | .12 | .12 | 0 | .12 | 0 | .12 | .12 | .12 | 0 | .12 | 0 | 0 |
| 250 | .12 | .12 | 0 | 0 | 0 | 0 | 0 | 0 | 0 | 0 | 0 | 0 | 0 | 0 | 0 | 0 | .12 | 0 | 0 | 0 | 0 |
| 251 | .12 | 0 | 0 | 0 | 0 | 0 | 0 | 0 | 0 | .12 | .12 | 0 | .12 | .12 | 0 | 0 | 0 | 0 | 0 | 0 | 0 |
| 252 | .12 | 0 | .12 | 0 | 0 | .12 | .12 | 0 | 0 | 0 | 0 | 0 | 0 | 0 | .12 | .12 | 0 | 0 | 0 | 0 | 0 |
| 253 | .12 | 0 | .12 | .12 | 0 | 0 | .12 | .12 | 0 | 0 | 0 | 0 | 0 | 0 | .12 | .12 | .12 | 0 | 0 | .12 | 0 |
| 254 | .12 | 0 | 0 | 0 | 0 | .12 | 0 | .12 | 0 | 0 | 0 | 0 | .12 | 0 | .12 | .12 | .12 | 0 | 0 | .12 | 0 |
| 255 | 0 | 0 | .12 | 0 | 0 | 0 | 0 | 0 | 0 | 0 | .12 | 0 | 0 | 0 | 0 | .12 | .12 | 0 | 0 | 0 | 0 |
| 256 | .12 | 0 | 0 | .12 | 0 | 0 | 0 | .12 | 0 | 0 | .12 | 0 | .12 | 0 | .12 | 0 | .12 | .12 | 0 | 0 | 0 |
| 257 | .12 | 0 | 0 | 0 | 0 | .12 | 0 | 0 | 0 | 0 | .12 | 0 | 0 | 0 | 0 | 0 | 0 | 0 | 0 | 0 | 0 |
| 258 | 0 | 0 | .12 | .12 | 0 | 0 | 0 | 0 | 0 | 0 | 0 | 0 | 0 | 0 | 0 | 0 | 0 | 0 | 0 | 0 | 0 |
| 259 | 0 | 0 | .12 | 0 | 0 | .12 | .12 | .12 | .12 | 0 | 0 | 0 | 0 | 0 | .12 | .12 | .12 | 0 | .12 | 0 | 0 |
| 260 | 0 | 0 | 0 | .12 | 0 | 0 | 0 | 0 | 0 | 0 | 0 | 0 | 0 | 0 | 0 | 0 | 0 | 0 | 0 | 0 | 0 |
| 261 | .12 | .12 | 0 | 0 | 0 | 0 | 0 | 0 | .12 | 0 | .12 | .12 | 0 | 0 | .12 | .12 | 0 | .12 | 0 | 0 | 0 |
| 262 | .12 | 0 | 0 | 0 | 0 | 0 | 0 | .12 | 0 | 0 | 0 | 0 | 0 | 0 | 0 | .12 | .12 | 0 | 0 | 0 | 0 |
| 263 | 0 | 0 | 0 | 0 | .12 | 0 | 0 | 0 | 0 | 0 | 0 | 0 | 0 | 0 | 0 | 0 | .12 | 0 | 0 | .12 | 0 |
| 264 | .12 | 0 | 0 | 0 | 0 | 0 | 0 | 0 | 0 | 0 | 0 | 0 | 0 | .12 | 0 | 0 | 0 | 0 | 0 | 0 | 0 |
| 265 | .12 | 0 | 0 | 1 | 0 | .12 | 1 | 0 | .12 | 0 | 0 | 0 | 0 | 0 | 0 | 0 | 0 | 0 | 0 | 0 | 0 |
| 266 | 0 | 0 | 0 | 0 | 0 | 0 | 0 | 0 | 0 | 0 | .12 | 0 | 0 | 0 | 0 | 0 | 0 | .12 | 0 | 0 | 0 |
| 267 | 0 | 0 | 0 | 0 | 0 | 0 | 0 | 0 | 0 | 0 | .12 | 0 | 0 | 0 | 0 | 0 | 0 | 0 | 0 | 0 | 0 |
| 268 | .12 | 0 | 0 | .12 | 0 | 0 | 0 | .12 | .12 | 0 | 0 | 0 | 0 | 0 | 0 | 0 | 0 | 0 | 0 | .12 | 0 |
| 269 | 0 | .12 | .12 | 0 | 0 | .12 | 0 | 0 | 0 | 0 | .12 | 1 | .12 | 0 | 0 | 0 | .12 | 0 | 0 | 0 | 0 |
| 270 | 0 | 0 | 0 | 0 | 0 | .12 | 0 | 0 | 0 | 0 | 0 | .12 | 0 | 0 | 0 | 0 | 0 | 0 | 0 | 0 | 0 |
| 271 | 1 | 0 | 0 | 0 | 0 | 0 | 0 | 0 | 0 | 0 | 0 | 0 | 0 | 0 | .12 | 0 | 0 | 0 | 0 | 0 | 0 |
| 272 | 0 | 0 | 0 | .12 | 0 | 0 | 0 | 0 | 0 | 0 | 0 | .12 | 0 | 0 | .12 | .12 | 0 | 0 | 0 | 0 | 0 |
| 273 | .12 | .12 | 0 | 0 | 0 | 0 | 1 | 1 | .12 | 0 | 0 | .12 | 0 | 0 | 0 | .12 | 0 | 0 | 0 | 0 | 0 |
| 274 | 0 | 0 | 0 | 0 | 0 | 0 | 0 | 0 | 0 | 0 | 0 | 0 | 0 | 0 | 0 | .12 | 0 | 0 | 0 | 0 | 0 |
| 275 | 0 | 0 | 0 | 0 | 0 | 0 | 0 | 0 | 0 | 0 | 0 | 0 | 0 | 0 | 0 | 0 | 0 | 0 | 0 | .12 | 0 |
| 276 | 0 | 0 | 0 | 0 | 0 | 0 | 0 | 0 | 0 | 0 | .12 | 0 | 0 | 0 | 0 | 0 | 0 | 0 | 0 | 0 | 0 |
| 277 | 0 | 0 | 0 | 0 | 0 | 0 | 0 | 0 | 0 | 0 | .12 | 0 | 0 | 0 | 0 | 0 | 0 | 0 | .12 | 0 | 0 |
| 278 | 0 | 0 | 0 | 0 | 0 | 0 | 0 | 0 | 0 | 0 | 0 | 0 | 0 | 0 | 0 | 0 | 0 | 0 | 0 | .12 | 0 |
| 279 | 0 | 0 | 0 | 0 | 0 | 0 | 0 | .12 | 0 | 0 | 0 | 0 | 0 | 0 | 0 | .12 | 0 | 0 | 0 | 0 | 0 |
| 280 | 0 | 0 | 0 | 0 | 0 | 0 | 0 | 0 | 0 | 0 | 0 | 0 | 0 | .12 | 0 | 0 | 0 | 0 | 0 | 0 | 0 |
| 281 | 0 | 0 | 0 | 0 | 0 | 0 | 0 | .12 | 0 | 0 | 0 | 0 | 0 | 0 | 0 | 0 | 0 | 0 | 0 | 0 | 0 |
| 282 | 0 | 0 | 0 | 0 | 0 | 0 | 0 | 0 | 0 | 0 | 0 | 0 | 0 | 0 | 0 | .12 | .12 | 0 | 0 | 0 | 0 |
| 283 | 0 | 0 | 0 | 0 | 0 | .12 | 0 | 0 | 0 | .12 | 0 | 0 | 0 | 0 | 0 | 0 | .12 | 0 | 0 | 0 | 0 |
| 284 | 0 | 0 | 0 | 0 | 0 | 0 | 0 | 0 | 0 | 0 | 0 | 0 | 0 | 0 | 0 | .12 | .12 | 0 | 0 | 0 | 0 |
| 285 | 0 | 0 | 0 | 0 | 0 | .12 | .12 | 0 | 0 | 0 | 0 | 0 | 0 | 0 | 0 | .12 | 0 | 0 | 0 | 0 | 0 |
| 286 | 0 | 0 | 0 | 0 | 0 | 0 | 0 | 0 | 0 | 0 | .12 | 0 | 0 | 0 | 0 | 0 | 0 | 0 | 0 | .12 | 0 |
| 287 | 0 | 0 | 0 | .12 | 0 | 0 | 1 | 0 | 0 | 0 | 0 | 0 | 0 | 0 | 0 | 1 | 1 | 0 | 0 | 0 | 0 |
| 288 | 0 | 1 | 0 | 0 | 0 | 0 | .12 | 0 | 0 | 0 | 0 | 0 | 0 | 0 | .12 | 0 | 0 | 0 | 0 | 1 | 0 |
| 289 | .12 | .12 | .12 | .12 | 0 | 0 | 1 | .12 | .12 | 0 | 0 | .12 | 0 | 0 | 0 | .12 | .12 | 0 | 0 | 0 | 0 |
| 290 | 0 | 0 | 0 | .12 | 0 | 0 | .12 | 0 | 0 | 0 | 0 | 0 | 0 | 0 | 0 | 0 | 1 | 0 | 0 | 0 | 0 |
| 291 | 1 | 0 | 0 | 0 | 0 | 0 | 0 | 0 | 0 | 0 | 0 | 0 | 0 | .12 | 0 | 0 | 0 | 0 | 0 | 0 | 0 |
| 292 | 0 | 0 | 0 | 0 | 0 | 0 | 0 | 0 | 0 | 0 | .12 | 0 | 0 | 0 | .12 | 0 | 0 | 0 | 0 | 0 | 0 |
| 293 | 1 | 1 | 1 | 0 | 0 | 0 | .12 | 0 | 0 | 0 | 0 | 1 | 0 | 0 | 0 | 0 | 1 | 0 | 0 | 0 | 0 |
| 294 | 0 | 0 | 0 | 0 | 0 | 0 | 0 | 0 | 0 | .12 | 0 | 0 | 0 | 0 | 0 | 0 | 0 | 0 | 0 | 0 | 0 |
| 295 | .12 | 0 | 0 | 0 | 0 | 0 | 0 | 0 | 0 | 0 | 0 | 0 | 1 | 0 | 0 | 0 | 0 | 0 | 0 | 1 | 0 |
| 296 | .12 | .12 | 0 | 1 | 0 | .12 | 1 | 0 | .12 | 0 | 0 | 0 | 0 | 0 | 0 | 0 | 0 | 0 | 0 | .12 | 0 |
| 297 | .12 | 0 | 0 | 0 | 0 | 0 | 0 | .12 | 0 | 0 | 0 | 0 | 0 | 0 | 0 | 0 | 0 | 0 | 0 | 0 | 0 |
| 298 | 0 | 0 | 0 | 0 | 0 | 0 | 0 | 0 | 0 | 0 | .12 | 0 | 0 | 0 | 0 | 0 | 0 | 0 | 0 | 0 | 0 |
| 299 | 0 | 0 | 0 | 0 | 0 | 0 | 0 | 0 | 0 | 1 | 0 | 0 | .12 | 0 | 0 | 0 | 0 | 0 | 0 | .12 | 0 |
| 300 | .12 | 1 | 0 | .12 | 0 | .12 | .12 | .12 | 0 | 0 | 1 | 1 | 0 | 0 | 0 | .12 | 0 | 0 | 0 | 0 | 0 |
| 301 | 0 | 0 | 0 | 0 | 0 | 0 | 0 | 0 | 0 | 0 | 0 | 0 | 0 | 0 | 0 | .12 | 0 | 0 | 0 | 0 | 0 |
| 302 | 0 | .12 | 0 | 0 | 0 | 0 | 1 | .12 | 0 | 0 | 0 | .12 | 0 | 0 | 0 | 0 | 0 | 0 | 0 | 0 | 0 |
| 303 | 0 | 0 | 0 | 0 | .12 | .12 | 0 | 0 | 0 | 1 | 1 | 0 | 0 | 0 | 0 | 0 | 0 | 0 | 0 | .12 | 0 |
| 304 | 0 | 0 | 0 | 0 | 0 | 0 | 0 | 0 | 0 | 0 | 0 | .12 | 0 | 0 | .12 | .12 | 0 | 0 | 0 | 0 | 0 |
| 305 | 0 | 0 | 0 | 0 | 0 | 0 | 0 | 0 | 0 | 0 | 0 | 0 | 0 | .12 | 0 | 0 | 0 | 0 | 0 | 0 | 0 |
| 306 | 0 | 0 | 0 | 0 | 0 | 0 | 0 | 0 | 0 | 0 | .12 | 0 | 0 | 0 | 0 | 0 | 0 | 0 | 0 | .12 | 0 |
| 307 | 0 | 0 | 0 | 0 | 0 | 0 | 0 | 0 | 0 | 0 | 0 | 0 | 0 | 0 | 0 | 0 | 0 | .12 | 0 | 0 | 0 |
| 308 | 0 | .12 | .12 | 0 | .12 | .12 | 0 | 0 | 0 | 0 | .12 | 0 | 0 | .12 | 0 | .12 | .12 | 0 | .12 | .12 | 0 |
| 309 | 0 | 0 | 0 | 0 | 0 | 0 | 0 | 0 | 0 | 0 | 0 | 0 | 0 | 0 | 0 | 0 | 0 | 0 | 0 | .12 | 0 |
| 310 | 0 | .12 | 0 | 0 | 0 | 0 | 0 | .12 | 0 | 0 | 0 | 0 | 0 | 0 | 0 | 0 | 0 | 0 | 0 | 0 | 0 |
| 311 | 0 | 0 | 0 | 0 | 0 | 0 | 0 | 0 | 0 | 0 | 0 | 0 | 0 | 0 | .12 | 0 | 0 | 0 | 0 | 0 | 0 |
| 312 | 0 | 0 | 0 | 0 | 0 | 0 | 0 | .12 | 0 | 0 | 0 | 0 | 0 | 0 | 0 | 0 | 0 | 0 | 0 | 0 | 0 |
| 313 | 0 | 0 | 0 | 0 | 0 | 0 | 0 | 0 | 0 | 0 | 0 | 0 | 0 | .12 | 0 | .12 | 0 | 0 | 0 | 0 | 0 |
| 314 | 0 | 0 | 0 | 0 | 0 | 0 | 0 | 0 | 0 | 0 | 0 | 0 | 0 | 0 | 0 | 0 | 0 | 0 | 0 | .12 | 0 |
| 315 | 0 | 0 | 0 | 0 | 0 | 0 | 0 | 0 | 0 | 0 | 0 | .12 | 0 | 0 | 0 | .12 | .12 | 0 | 0 | 0 | 0 |
| 316 | 0 | 0 | 0 | 0 | 0 | 0 | 0 | .12 | 0 | 0 | 0 | 0 | 0 | 0 | 0 | 0 | 0 | 0 | 0 | 0 | 0 |
| 317 | 0 | .12 | 0 | 0 | 0 | 1 | 1 | 0 | 1 | 0 | .12 | 1 | 0 | 0 | 0 | .12 | 0 | 0 | .12 | 0 | 0 |
| 318 | 1 | 1 | 0 | 1 | 0 | 0 | .12 | 0 | 0 | 0 | 0 | 0 | 0 | 0 | 0 | 1 | .12 | 0 | 0 | 0 | 0 |
| 319 | 0 | 0 | 0 | 0 | 0 | 0 | 0 | 0 | 0 | 0 | 0 | 0 | 0 | 0 | 0 | 0 | 0 | .12 | 0 | 0 | 0 |
| 320 | 0 | 0 | 0 | 0 | 0 | 0 | 0 | 0 | 0 | .12 | 0 | 0 | 0 | 0 | 0 | 0 | 0 | 0 | 0 | .12 | 0 |
| 321 | 0 | 0 | 0 | .12 | 0 | 0 | .12 | 0 | 0 | 0 | 0 | 0 | 0 | 0 | 0 | 0 | 0 | 0 | 0 | 0 | 0 |
| 322 | .12 | .12 | 0 | 0 | 0 | 0 | 0 | .12 | 0 | 0 | .12 | .12 | .12 | 0 | .12 | .12 | .12 | 0 | 0 | .12 | 0 |
| 323 | 0 | 0 | 0 | 0 | 0 | 0 | 0 | 0 | 0 | 1 | .12 | 0 | 0 | 0 | 0 | 0 | 0 | 0 | 0 | 0 | 0 |
| 324 | 0 | 0 | 0 | .12 | 0 | 0 | 0 | .12 | 0 | 0 | 0 | 0 | 0 | 0 | .12 | 0 | 0 | 0 | 0 | 0 | 0 |
| 325 | 0 | 0 | 0 | .12 | 0 | 0 | 1 | 0 | 0 | 0 | 0 | 0 | 0 | 0 | 1 | 0 | 0 | 0 | 0 | 0 | 0 |
| 326 | .12 | 0 | .12 | 0 | 0 | 0 | .12 | .12 | 0 | 0 | 0 | 0 | 0 | 0 | 0 | .12 | 0 | 0 | 0 | 0 | 0 |
| 327 | 0 | 1 | 0 | 0 | 0 | 0 | 0 | 0 | 0 | 0 | .12 | 1 | 0 | .12 | 0 | 0 | 0 | 0 | .12 | 0 | 0 |
| 328 | 0 | 0 | 0 | 0 | 0 | 0 | 0 | 1 | 0 | 0 | .12 | 0 | 0 | 0 | 0 | 0 | .12 | 0 | 0 | 0 | 0 |
| 329 | 0 | 0 | 0 | 0 | .12 | 0 | 0 | .12 | 0 | 0 | 0 | 0 | 0 | 0 | .12 | 0 | 0 | 0 | 0 | 0 | 0 |
| 330 | 0 | 0 | 0 | .12 | 0 | 0 | .12 | .12 | 0 | 0 | 0 | 0 | 0 | 0 | 1 | .12 | .12 | 0 | 0 | 0 | 0 |
| 331 | 1 | .12 | 0 | 0 | .12 | 1 | 0 | 0 | 0 | 0 | 0 | .12 | 0 | 0 | 0 | 0 | 0 | 0 | 0 | 0 | 0 |
| 332 | 0 | 0 | 0 | 0 | 0 | 0 | 0 | .12 | 0 | 0 | 0 | 0 | 0 | 0 | 0 | 0 | 0 | 0 | 0 | 0 | 0 |
| 333 | 0 | .12 | 0 | 0 | 0 | 0 | 0 | 0 | 0 | 0 | 0 | 0 | 0 | 0 | 0 | 0 | 0 | 0 | 0 | 0 | 0 |
| 334 | 0 | 0 | 0 | 0 | 0 | 0 | 0 | 0 | 0 | .12 | 0 | 0 | 0 | 0 | 0 | 0 | 0 | 0 | 0 | .12 | 0 |
| 335 | 0 | 0 | 0 | 0 | 0 | 0 | 0 | 0 | 0 | 0 | 0 | 0 | 0 | 0 | 0 | 0 | 0 | 0 | 0 | .12 | 0 |
| 336 | 0 | .12 | 0 | 0 | 0 | .12 | .12 | 0 | 0 | 0 | 0 | .12 | .12 | 0 | 0 | 0 | 0 | 0 | .12 | 0 | 0 |
| 337 | 0 | 0 | .12 | 0 | 0 | 0 | 0 | 0 | 0 | 0 | 0 | 0 | 0 | .12 | 0 | .12 | 0 | .12 | 0 | 0 | 0 |
| 338 | .12 | 0 | 0 | 0 | .12 | 0 | 0 | 0 | 0 | 0 | 0 | 0 | 0 | 0 | 0 | 0 | 0 | 0 | 0 | .12 | 0 |
| 339 | 0 | 0 | 0 | .12 | 0 | 0 | 0 | 0 | 0 | 0 | 0 | 0 | 0 | 0 | .12 | 0 | 0 | 0 | 0 | 0 | 0 |
| 340 | 0 | 0 | 0 | 0 | 0 | .12 | 0 | 0 | 0 | 0 | 0 | 0 | 0 | 0 | 0 | 0 | 0 | 0 | 0 | 0 | 0 |
| 341 | 0 | 0 | 0 | 0 | 0 | .12 | 0 | 0 | 0 | 0 | 0 | 0 | 0 | 0 | 0 | 0 | 0 | 0 | 0 | .12 | 0 |
| 342 | 0 | .12 | 0 | 0 | 0 | 0 | .12 | 0 | 0 | 0 | 0 | 0 | 0 | 0 | 0 | 0 | 0 | .12 | 0 | 0 | 0 |
| 343 | 0 | 0 | 0 | 0 | 0 | 0 | 0 | 0 | 0 | .12 | 0 | 0 | 0 | 0 | 0 | 0 | 0 | 0 | 0 | .12 | 0 |
| 344 | 0 | 0 | 0 | 0 | 0 | 0 | 0 | 0 | 0 | 0 | .12 | 0 | 0 | 0 | 0 | 0 | 0 | 0 | 0 | 0 | 0 |
| 345 | .12 | 0 | 0 | 0 | 0 | 0 | 0 | 0 | 0 | 0 | 0 | 0 | 0 | 0 | 0 | 0 | 0 | 0 | 0 | 0 | 0 |
| 346 | 0 | 0 | 0 | 0 | 0 | 0 | 0 | 0 | .12 | 0 | 0 | 0 | 0 | 0 | 0 | 0 | 0 | 0 | 0 | 0 | 0 |
| 347 | 0 | .12 | 0 | .12 | 0 | 0 | 0 | .12 | .12 | 0 | 0 | .12 | 0 | 0 | .12 | 0 | 0 | 0 | 0 | 0 | 0 |
| 348 | .12 | 0 | 0 | 0 | .12 | 0 | 0 | 0 | 0 | 0 | 0 | 0 | 0 | 0 | 0 | .12 | 0 | 0 | 0 | 0 | 0 |
| 349 | 0 | 0 | 0 | 0 | 0 | 0 | 0 | 0 | 0 | .12 | 0 | 0 | 0 | 0 | 0 | 0 | 0 | 0 | 0 | .12 | 0 |
| 350 | 0 | 0 | 0 | 0 | 0 | 0 | 0 | .12 | 0 | 0 | 0 | 0 | 0 | 0 | 0 | 0 | 0 | 0 | 0 | 0 | 0 |
| 351 | .12 | 0 | 0 | 0 | 0 | 0 | 0 | .12 | 0 | 0 | 0 | 0 | 0 | 0 | 0 | 0 | 0 | 0 | 0 | 0 | 0 |
| 352 | 0 | 0 | 0 | 0 | 0 | .12 | 0 | 0 | 0 | 0 | 0 | 0 | 0 | .12 | 0 | 0 | 0 | 0 | 0 | 0 | 0 |
| 353 | 0 | 0 | 0 | 0 | 0 | 0 | 0 | 0 | 0 | 0 | 0 | 0 | 0 | 0 | 0 | 0 | 0 | .12 | .12 | 0 | 0 |
| 354 | 0 | 0 | 0 | 0 | 0 | 0 | 0 | 0 | 0 | 0 | 0 | 0 | 0 | 0 | 0 | 0 | .12 | 0 | 0 | 0 | 0 |
| 355 | 0 | 0 | 0 | 0 | 0 | 0 | 0 | 0 | .12 | 0 | 0 | 0 | 0 | 0 | 0 | 0 | 0 | 0 | 0 | 0 | 0 |
| 356 | 0 | 0 | 0 | 0 | 0 | 0 | 0 | .12 | 0 | 0 | 0 | 0 | 0 | 0 | 0 | .12 | 0 | 0 | 0 | 0 | 0 |
| 357 | 0 | 0 | 0 | 0 | 0 | 0 | 0 | .12 | 0 | 0 | 0 | 0 | 0 | 0 | 0 | 0 | 0 | 0 | 0 | 0 | 0 |
| 358 | 0 | 0 | 0 | 0 | 0 | 0 | 0 | 0 | 0 | 0 | 0 | 0 | 0 | 0 | 0 | 0 | 0 | .12 | 0 | 0 | 0 |
| 359 | 0 | 0 | .12 | 0 | 0 | 0 | 0 | .12 | 0 | 0 | 0 | 0 | 0 | 0 | 0 | 0 | 0 | 0 | 0 | 0 | 0 |
| 360 | 0 | 0 | 0 | 0 | 0 | 0 | 0 | .12 | 0 | 0 | 0 | 0 | 0 | 0 | 0 | .12 | 0 | 0 | 0 | 0 | 0 |
| 361 | 0 | 0 | 0 | 0 | .12 | 0 | 0 | 0 | 0 | 0 | 0 | 0 | 0 | 0 | 0 | 0 | .12 | 0 | 0 | 0 | 0 |
| 362 | 0 | 0 | 0 | 0 | 0 | 0 | 0 | 0 | 0 | 0 | .12 | 0 | .12 | 0 | 0 | 0 | .12 | 0 | 0 | 0 | 0 |
| 363 | 0 | 0 | 0 | 0 | 0 | 0 | .12 | 0 | 0 | 0 | 0 | 0 | 0 | 0 | 0 | 0 | 0 | 0 | 0 | 0 | 0 |
| 364 | .12 | 0 | 0 | 0 | 0 | 0 | 0 | .12 | 0 | 0 | 0 | 0 | 0 | 0 | 0 | .12 | 0 | 0 | 0 | 0 | 0 |
| 365 | 0 | 0 | 0 | 0 | 0 | 0 | 0 | 0 | 0 | .12 | 0 | 0 | 0 | 0 | 0 | 0 | 0 | 0 | 0 | .12 | 0 |
| 366 | .12 | 0 | 0 | 0 | .12 | 0 | 0 | 0 | 0 | 0 | .12 | 0 | 0 | 0 | 0 | .12 | .12 | 0 | 0 | .12 | 0 |
| 367 | 0 | 0 | 0 | 0 | 0 | 0 | .12 | 0 | 0 | 0 | 0 | 0 | 0 | 0 | 0 | 0 | 0 | 0 | 0 | 0 | 0 |
| 368 | 0 | 0 | 0 | 0 | 0 | 0 | 0 | .12 | 0 | 0 | 0 | 0 | 0 | 0 | 0 | 0 | 0 | 0 | 0 | 0 | 0 |
| 369 | 0 | 0 | 0 | 0 | 0 | 0 | 0 | 0 | 0 | 0 | 0 | .12 | 0 | 0 | 0 | 0 | 0 | 0 | 0 | .12 | 0 |
| 370 | 0 | 0 | 0 | 0 | 0 | 0 | 0 | 0 | 0 | 0 | 0 | 0 | 0 | 0 | .12 | 0 | 0 | 0 | 0 | 0 | 0 |
| 371 | 0 | 0 | 0 | 0 | 0 | 0 | 0 | 0 | 0 | 0 | 0 | 0 | .12 | 0 | 0 | 0 | 0 | 0 | 0 | 0 | 0 |
| 372 | 0 | 0 | 0 | 0 | 0 | 0 | 0 | 0 | 0 | .12 | 0 | 0 | 0 | 0 | 0 | 0 | 0 | 0 | 0 | 0 | 0 |
| 373 | 0 | 0 | 0 | 0 | 0 | 0 | 0 | 0 | 0 | 0 | 0 | 0 | 0 | .12 | 0 | 0 | 0 | 0 | 0 | 0 | 0 |
| 374 | 0 | .12 | 0 | 0 | 0 | 0 | 0 | 0 | 0 | 0 | 0 | .12 | 0 | 0 | 0 | .12 | .12 | 0 | 0 | 0 | 0 |
| 375 | .12 | 0 | 0 | .12 | 0 | 0 | 0 | 0 | 0 | 0 | 0 | 0 | 0 | 0 | .12 | 0 | .12 | 0 | 0 | .12 | 0 |
| 376 | 0 | 0 | 0 | 0 | 0 | 0 | 0 | 0 | 0 | 0 | 0 | 0 | 0 | .12 | 0 | 0 | 0 | 0 | 0 | 0 | 0 |
| 377 | 0 | 0 | 0 | 0 | 0 | 0 | 0 | .12 | 0 | 0 | 0 | 0 | 0 | 0 | 0 | 0 | 0 | 0 | 0 | 0 | 0 |
| 378 | 0 | 0 | 0 | 0 | 0 | 0 | 0 | 0 | 0 | 0 | .12 | 0 | 0 | 0 | 0 | 0 | 0 | 0 | 0 | 0 | 0 |
| 379 | 0 | 0 | 0 | .12 | 0 | 0 | 0 | 0 | 0 | 0 | 0 | 0 | 0 | 0 | 0 | 0 | 0 | 0 | 0 | 0 | 0 |
| 380 | 0 | 0 | 0 | 0 | 0 | .12 | 0 | 0 | 0 | 0 | 0 | 0 | 0 | 0 | 0 | 0 | 0 | 0 | 0 | 0 | 0 |
| 381 | 0 | 0 | 0 | 0 | 0 | 0 | 0 | 0 | 0 | 0 | 0 | 0 | 0 | 0 | .12 | 0 | 0 | 0 | 0 | 0 | 0 |
| 382 | 0 | 0 | 0 | 0 | 0 | 0 | 0 | 0 | 0 | 0 | .12 | 0 | 0 | 0 | 0 | 0 | 0 | 0 | 0 | 0 | 0 |
| 383 | 0 | 0 | .12 | 0 | 0 | 0 | 0 | 0 | 0 | 0 | 0 | 0 | 0 | 0 | 0 | 0 | 0 | 0 | 0 | .12 | 0 |
| 384 | .12 | 0 | 0 | 0 | 0 | 0 | 0 | 0 | 0 | 0 | 0 | 0 | 0 | 0 | 0 | 0 | 0 | 0 | 0 | 0 | 0 |
| 385 | 1 | .12 | 0 | 0 | 0 | 0 | 0 | 0 | 0 | 0 | 0 | 0 | 0 | 0 | 0 | 0 | 0 | 0 | 0 | 0 | 0 |
| 386 | 0 | 0 | 0 | 0 | 0 | 0 | 0 | 0 | 0 | 0 | 0 | 0 | 0 | 0 | 0 | 0 | 0 | 0 | .12 | 0 | 0 |
| 387 | 0 | 0 | 0 | 0 | 0 | 0 | 0 | 0 | 0 | 0 | 0 | 0 | .12 | 0 | 0 | 0 | 0 | 0 | 0 | 0 | 0 |
| 388 | 0 | 0 | 0 | 0 | 0 | 0 | 0 | 0 | 0 | 0 | 0 | 0 | 0 | 0 | 0 | .12 | .12 | 0 | 0 | .12 | 0 |
| 389 | .12 | 1 | 0 | .12 | 0 | 0 | .12 | 0 | 0 | 0 | .12 | 0 | 0 | 0 | 0 | .12 | 0 | 0 | 0 | 0 | 0 |
| 390 | 0 | .12 | 0 | 0 | 0 | 0 | 1 | 0 | 0 | .12 | 0 | 1 | 0 | 0 | 0 | 0 | 0 | 0 | 0 | .12 | 0 |
| 391 | 0 | .12 | 0 | 0 | .12 | 0 | 0 | 0 | 0 | 0 | .12 | 1 | 0 | 0 | 0 | 0 | 0 | 0 | 0 | 0 | 0 |
| 392 | 0 | 1 | 0 | .12 | 0 | .12 | 0 | 1 | .12 | 0 | 0 | .12 | 0 | 0 | 0 | 0 | 0 | 0 | 0 | 0 | 0 |
| 393 | 0 | 0 | 0 | 0 | 0 | 0 | 0 | 0 | 0 | .12 | 0 | 0 | 0 | 0 | 0 | 0 | 0 | 0 | 0 | .12 | 0 |
| 394 | 0 | 0 | 0 | 0 | 0 | 0 | 0 | .12 | 0 | 0 | 0 | 0 | 0 | 0 | 0 | 0 | 0 | 0 | 0 | 0 | 0 |
| 395 | 0 | 0 | 0 | 0 | 0 | 0 | 0 | 0 | 0 | .12 | 0 | 0 | 0 | 0 | 0 | 0 | 0 | 0 | 0 | .12 | 0 |
| 396 | 0 | .12 | 0 | 0 | 0 | 0 | 0 | 0 | 0 | 0 | 0 | 0 | 0 | 0 | 0 | 0 | .12 | 0 | .12 | .12 | 0 |
| 397 | 0 | 0 | 0 | 0 | 0 | 0 | 0 | 0 | 0 | 0 | .12 | 0 | 0 | 0 | 0 | 0 | 0 | 0 | 0 | 0 | 0 |
| 398 | 0 | 1 | 0 | 1 | 0 | 0 | .12 | 0 | 0 | 0 | 0 | 0 | 0 | 0 | 0 | .12 | 0 | 0 | 0 | 0 | 0 |
| 399 | 0 | 1 | .12 | .12 | 0 | .12 | 1 | 0 | 0 | 0 | 0 | 0 | 0 | 0 | .12 | .12 | 0 | 0 | 0 | 0 | 0 |
| 400 | 0 | 0 | 0 | 0 | 0 | 0 | 0 | .12 | 0 | 0 | 0 | 0 | 0 | 0 | 0 | 0 | 0 | 0 | 0 | 0 | 0 |
| 401 | 0 | 0 | 0 | 0 | 0 | 0 | 0 | 0 | 0 | 0 | .12 | 0 | 0 | .12 | 0 | 0 | 0 | .12 | 0 | 0 | 0 |
| 402 | 0 | 0 | 1 | 1 | 0 | 0 | .12 | 0 | 0 | .12 | .12 | 0 | 0 | 0 | 0 | 0 | 0 | 0 | 0 | 1 | 0 |
| 403 | 0 | .12 | 0 | 0 | 0 | 0 | 0 | 0 | 0 | 0 | 0 | 1 | 0 | 0 | 1 | 0 | 0 | 0 | 0 | .12 | 0 |
| 404 | 1 | 0 | 0 | 1 | 0 | 0 | 1 | .12 | 0 | 0 | 0 | 0 | .12 | 0 | 0 | 0 | .12 | 0 | 0 | .12 | 0 |
| 405 | .12 | 1 | 0 | .12 | 0 | .12 | .12 | 0 | 0 | 0 | 0 | 1 | 0 | 0 | .12 | .12 | .12 | 0 | 0 | 0 | 0 |
| 406 | 0 | 0 | 0 | 0 | 0 | 0 | 0 | 0 | 0 | .12 | 0 | 0 | 0 | 0 | 0 | 0 | 0 | 0 | 0 | 0 | 0 |
| 407 | .12 | 0 | 0 | 0 | .12 | 0 | .12 | 0 | 0 | .12 | 0 | 0 | 0 | 0 | 0 | 0 | .12 | 0 | 0 | .12 | 0 |
| 408 | 1 | .12 | .12 | .12 | .12 | 0 | 0 | 0 | 0 | 0 | 0 | .12 | 0 | 0 | 0 | .12 | .12 | 0 | 0 | 0 | 0 |
| 409 | .12 | 0 | 0 | 0 | 0 | 0 | 0 | 0 | 0 | 0 | 0 | 0 | 0 | 0 | 0 | .12 | 0 | 0 | 0 | 0 | 0 |
| 410 | 0 | 0 | 0 | 0 | 0 | 0 | 0 | 0 | 0 | .12 | 0 | 0 | 0 | 0 | 0 | 0 | 0 | 0 | 0 | .12 | 0 |
| 411 | 0 | .12 | 1 | 0 | 0 | .12 | .12 | 0 | .12 | 0 | 0 | .12 | 0 | 0 | 0 | .12 | .12 | 0 | 0 | 0 | 0 |
| 412 | .12 | .12 | 0 | 0 | 0 | 0 | 0 | 0 | .12 | 0 | 1 | .12 | 0 | 0 | 0 | .12 | .12 | 0 | 1 | 0 | 0 |
| 413 | 0 | 0 | 0 | 0 | 0 | 0 | 0 | 0 | 0 | 0 | .12 | 0 | .12 | 0 | 0 | 0 | 0 | 0 | 0 | .12 | 0 |
| 414 | 0 | 0 | 0 | 0 | 0 | 0 | 0 | 0 | 0 | 0 | .12 | 0 | .12 | 0 | 0 | 0 | 0 | 0 | 0 | 0 | 0 |
| 415 | 0 | 1 | 0 | 0 | 0 | 1 | 1 | 0 | 0 | 0 | 0 | 1 | 0 | 0 | 0 | 0 | 0 | 0 | 0 | .12 | 0 |
| 416 | 0 | 0 | 0 | .12 | 0 | 0 | .12 | 0 | 0 | 0 | .12 | 0 | 0 | 0 | 0 | .12 | 0 | 0 | 0 | 0 | 0 |
| 417 | .12 | .12 | 0 | 0 | 0 | .12 | .12 | 0 | 0 | 0 | 0 | 0 | 0 | 0 | .12 | 0 | 0 | 0 | 0 | .12 | 0 |
| 418 | 1 | 0 | 0 | .12 | 0 | 0 | .12 | 0 | 0 | 0 | 0 | 1 | 0 | 0 | 0 | 1 | 0 | 0 | 0 | 0 | 0 |
| 419 | 0 | 0 | 0 | 0 | 0 | 0 | 0 | .12 | 0 | 0 | 0 | 0 | 0 | 0 | 0 | 0 | 0 | 0 | 0 | 0 | 0 |
| 420 | .12 | .12 | 0 | 0 | 0 | 1 | .12 | .12 | 0 | 0 | 0 | 1 | 0 | 0 | 0 | 0 | 0 | 0 | 0 | 0 | 0 |
| 421 | 0 | 0 | 0 | 0 | 0 | 0 | 0 | 0 | 1 | .12 | 0 | 0 | 0 | 0 | 1 | 0 | 0 | 0 | .12 | .12 | 0 |
| 422 | 0 | 0 | 0 | 0 | 0 | .12 | 0 | 0 | 0 | .12 | 0 | 0 | .12 | 0 | 0 | 0 | 0 | 0 | 0 | 0 | 0 |
| 423 | 0 | .12 | 0 | 0 | 0 | 0 | 0 | 0 | 0 | 0 | 0 | .12 | 0 | 0 | 0 | 0 | .12 | 0 | 0 | 0 | 0 |
| 424 | .12 | 1 | 0 | 1 | 0 | .12 | .12 | 0 | 0 | 0 | 0 | 1 | 0 | 0 | 0 | 0 | 0 | .12 | 0 | 0 | 0 |
| 425 | 0 | 0 | .12 | 0 | 0 | 0 | 0 | 0 | 0 | 0 | 0 | 0 | 0 | 0 | 0 | 0 | 0 | 0 | 0 | 0 | 0 |
| 426 | .12 | 0 | 0 | 0 | 0 | 0 | 0 | 0 | 0 | .12 | 0 | 0 | 0 | 0 | 0 | .12 | .12 | 0 | 0 | .12 | 0 |
| 427 | .12 | .12 | 0 | 0 | 0 | 0 | 1 | 0 | 0 | 0 | .12 | .12 | 0 | 0 | 0 | 0 | 0 | 0 | 0 | 0 | 0 |
| 428 | 1 | .12 | .12 | 0 | 0 | .12 | .12 | .12 | 0 | .12 | .12 | .12 | 0 | .12 | 0 | .12 | .12 | 0 | .12 | .12 | 0 |
| 429 | 0 | 0 | 0 | 0 | 0 | 0 | 0 | 0 | 0 | 0 | .12 | 0 | 0 | 0 | 0 | 0 | 0 | .12 | 0 | 0 | 0 |
| 430 | 0 | .12 | 0 | 0 | 0 | .12 | 0 | 0 | 0 | 0 | 0 | .12 | 0 | 0 | 0 | 0 | 0 | 0 | 0 | 0 | 0 |
| 431 | 0 | .12 | 0 | .12 | 0 | .12 | .12 | 0 | 0 | 0 | 0 | 0 | .12 | 0 | 0 | 0 | 0 | 0 | 0 | 0 | 0 |
| 432 | 0 | 0 | 0 | 0 | 0 | 0 | 0 | 0 | 0 | 0 | .12 | .12 | 0 | 0 | 0 | 0 | .12 | 0 | 0 | 0 | 0 |
| 433 | .12 | 0 | 0 | 0 | 0 | 0 | 0 | 0 | 0 | 0 | .12 | 0 | 0 | 0 | 0 | 0 | 0 | 0 | 0 | 0 | 0 |
| 434 | 0 | 0 | .12 | .12 | 0 | 0 | .12 | .12 | 0 | 0 | 0 | 0 | 0 | 0 | 0 | 0 | 0 | 0 | 0 | 0 | 0 |
| 435 | .12 | .12 | .12 | .12 | .12 | .12 | .12 | .12 | 0 | .12 | .12 | .12 | .12 | 0 | 0 | .12 | .12 | 0 | .12 | .12 | 0 |
| 436 | 0 | 0 | 0 | 0 | 0 | 0 | 0 | 0 | 0 | 0 | 0 | 0 | 0 | 0 | 0 | .12 | 0 | 0 | 0 | 0 | 0 |
| 437 | 0 | 0 | 0 | 0 | 0 | 0 | 0 | 0 | 0 | .12 | .12 | 0 | 0 | 0 | 0 | 0 | .12 | 0 | 0 | .12 | 0 |
| 438 | .12 | .12 | 0 | 0 | 0 | .12 | .12 | 0 | 0 | 0 | 0 | 0 | .12 | 0 | 0 | .12 | 0 | 0 | 0 | 0 | 0 |
| 439 | .12 | .12 | .12 | .12 | 0 | .12 | .12 | .12 | .12 | 0 | 0 | .12 | .12 | 0 | .12 | 0 | 0 | .12 | 0 | .12 | 0 |
| 440 | 0 | 0 | 0 | 0 | 0 | 0 | 0 | .12 | 0 | 0 | 0 | 0 | 0 | 0 | 0 | 0 | 0 | 0 | 0 | 0 | 0 |
| 441 | 0 | 0 | 0 | 0 | 0 | 0 | 0 | .12 | 0 | 0 | 0 | 0 | 0 | 0 | 0 | 0 | 0 | 0 | 0 | 0 | 0 |
| 442 | 0 | 0 | 0 | 0 | 0 | 0 | 0 | 0 | 0 | 0 | 0 | 0 | 0 | .12 | 0 | .12 | .12 | 0 | 0 | 0 | 0 |
| 443 | .12 | 0 | 0 | 0 | 0 | 0 | 0 | 0 | 0 | 0 | 0 | 0 | 0 | 0 | 0 | .12 | 0 | 0 | 0 | 0 | 0 |
| 444 | .12 | 0 | 0 | 0 | 0 | 0 | 0 | 0 | .12 | 0 | 0 | 0 | 0 | 0 | 0 | 0 | 0 | 0 | .12 | 0 | 0 |
| 445 | 0 | 0 | 0 | 0 | 0 | 0 | .12 | 0 | 0 | 0 | 0 | 0 | 0 | 0 | 0 | 0 | .12 | 0 | 0 | 0 | 0 |
| 446 | 0 | 0 | 0 | 0 | 0 | 0 | 0 | 0 | 0 | 0 | 0 | 0 | 0 | .12 | 0 | .12 | 0 | 0 | 0 | 0 | 0 |
| 447 | 0 | 0 | 0 | 0 | 0 | 0 | 0 | 0 | 0 | 0 | .12 | 0 | 0 | 0 | 0 | 0 | 0 | 0 | 0 | 0 | 0 |
| 448 | 0 | 0 | 0 | 0 | 0 | .12 | .12 | 0 | .12 | 0 | 0 | .12 | 0 | 0 | 0 | 0 | 0 | 0 | 0 | 0 | 0 |
| 449 | 0 | .12 | 0 | 0 | 0 | 0 | 0 | .12 | 0 | 0 | .12 | 0 | 0 | 0 | 0 | .12 | 0 | 0 | 0 | 0 | 0 |
| 450 | 0 | 0 | 0 | 0 | 0 | 0 | 0 | 0 | 0 | 0 | .12 | 0 | 0 | 0 | 0 | 0 | 0 | 0 | 0 | 0 | 0 |
| 451 | 0 | 0 | 0 | 0 | 0 | .12 | .12 | 0 | 0 | 0 | 0 | 0 | 0 | 0 | 0 | 0 | 0 | 0 | 0 | .12 | 0 |
| 452 | 0 | 0 | 0 | 0 | 0 | 0 | 0 | 0 | 0 | 0 | 0 | 0 | 0 | 0 | 0 | .12 | .12 | 0 | 0 | 0 | 0 |
| 453 | 0 | .12 | 0 | 0 | 0 | 0 | 0 | 0 | 0 | 0 | 0 | 0 | 0 | 0 | 0 | 0 | 0 | 0 | .12 | 0 | 0 |
| 454 | 0 | 0 | 0 | 0 | 0 | 0 | 0 | 0 | 0 | .12 | 0 | 0 | 0 | 0 | 0 | 0 | 0 | 0 | 0 | .12 | 0 |
| 455 | 0 | .12 | 0 | 0 | 0 | 0 | 0 | 0 | 0 | 0 | 0 | 0 | .12 | 0 | 0 | .12 | 0 | 0 | 0 | 0 | 0 |
| 456 | 0 | .12 | 0 | 0 | 0 | 0 | 0 | 0 | 0 | 0 | 0 | .12 | 0 | 0 | 0 | .12 | 0 | 0 | 0 | 0 | 0 |
| 457 | 0 | 0 | 0 | 0 | 0 | 0 | 0 | 0 | 0 | 0 | .12 | 0 | 0 | 0 | 0 | 0 | 0 | 0 | 0 | 0 | 0 |

### Example 6: E. coli Fermentation of Designed B13GTs

E. coli strains that control B13GT expression with a constitutive promoter were made for the top B13GTs from Example 3. The E. coli strains were grown in 1 liter fermenters with a glycerol feed for 48 hours. The fermentations of strains expressing AA37959, AA40205, AA40232, and AA40243 yielded 171-, 60-, 182-, and 190-grams wet cell mass per liter fermentation. Twenty to thirty grams of cells from each fermentation were resuspended and lysed with a homogenizer. All lysates were tested and confirmed to have B13GT activity. The clarified lysate was purified using IMAC. The resulting purified protein samples of AA37959, AA40205, AA40232, and AA40243 had protein yields of 0.47, 0.15, 1.20 and 1.42 mg/ml, respectively.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. An engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to an amino acid sequence selected from the group consisting of SEQ ID NOs: 4-517, 2, and 3.

2. The engineered beta-1,3-glycosyltransferase polypeptide of claim 1, comprising an amino acid sequence that is identical to an amino acid sequence selected from the group consisting of SEQ ID NOs: 4-517, 2, and 3.

3. An engineered beta-1,3-glycosyltransferase polypeptide that has a score greater than 332 when scored by the PSSM shown in Table 8, wherein the engineered beta-1,3-glycosyltransferase polypeptide has been aligned to SEQ ID NO: 2 prior to scoring.

4. An engineered beta-1,3-glycosyltransferase polypeptide that has a score greater than 54.5 when scored by the PSSM shown in Table 9, wherein the engineered beta-1,3-glycosyltransferase polypeptide has been aligned to SEQ ID NO: 2 prior to scoring.

5. An engineered beta-1,3-glycosyltransferase polypeptide that has a score greater than 58.3 when scored by the PSSM shown in Table 10, wherein the engineered beta-1,3-glycosyltransferase polypeptide has been aligned to SEQ ID NO: 2 prior to scoring.

6. A polypeptide having the sequence of:
MPNTXXTXXRXXXXXXFPVPFPGHXNPMLQLANXLYXXGFXIXIXX
XXFNXPKTSXYPHFXFXXXXDXXPQXXXLRNLPTXGPGXGARIPX
INXHGXXXXXXXLXXXXXXXXXDXXXXCXIXDALWYFXXXVXXXL
XXXXLVLXTXSLFNFXXLXXLPXFXXXGXLXXXXXXXXXXXXXGX
PXLXXXDIXXAYSHXXEXKPILXKMXXXTXXXSGXIWNSXKELEE
SELETXXREIPAPSFLXPLPKHXXASSXSLLDXDXXXXXWLXXQX
XXSVXYVXFGSQSXXXXXXXXXXXXGLXXSXXXXLWLVRPGFXKG
XXWXXXXPXGXXGXXXRXVKSXPQQEVLAHXAIGAFWTHGGWNGT
XEXVCEGVPMIXSXFGLDQPLNAXYMXXXXXXGXYLXXGXXXXXX
XXAXXXXXXXXXGXXXXXXAXXLXXKXDXSXXXGGSSYESLEXLX
SYISSL
wherein residue 5 is N or S
wherein residue 6 is P or T
wherein residue 8 is T or V
wherein residue 9 is R or V
wherein residue 11 is R or D or E or H or K or S or T
wherein residue 12 is R or D or L or K
wherein residue 13 is R or H or T or W
wherein residue 14 is I or V
wherein residue 15 is A or I or V
wherein residue 16 is I or L or M
wherein residue 25 is I or L or W or V
wherein residue 34 is L or V
wherein residue 37 is R or S
wherein residue 38 is R or K
wherein residue 41 is A or R or N or D or Q or E or K or S
wherein residue 43 is I or T or V
wherein residue 45 is I or L or F
wherein residue 46 is H or Y
wherein residue 47 is M or T
wherein residue 48 is A or R or N or Q or E or K or S or T
wherein residue 51 is A or R or L or K
wherein residue 56 is N or L
wherein residue 61 is Q or E or T
wherein residue 63 is R or E or I or L or K or V
wherein residue 64 is R or N or D or Q or E or L or F or P or S or T or Y
wherein residue 65 is I or L or F or T or V
wherein residue 66 is I or L
wherein residue 68 is R or N or E
wherein residue 69 is D or E
wherein residue 72 is D or P or T
wherein residue 73 is D or E
wherein residue 74 is W or Y
wherein residue 81 is H or T
wherein residue 85 is A or V
wherein residue 91 is Q or I or L or V
wherein residue 94 is Q or E or K
wherein residue 97 is A or K
wherein residue 98 is A or D or E or K or P
wherein residue 99 is A or R or Q or E or K or P or T or V
wherein residue 100 is L or F
wherein residue 101 is R or E or L or K or Y
wherein residue 102 is A or R or D or Q or E or K
wherein residue 103 is A or E or I or L or K or M or T or V
wherein residue 105 is R or Q or E or L or K or S
wherein residue 106 is A or R or N or D or Q or E or I or L or K or S
wherein residue 107 is A or C or E or I or L or M or F or Y or V
wherein residue 108 is I or L or K or M
wherein residue 109 is A or R or N or D or Q or E or L or K or S
wherein residue 110 is A or N or D or E or S
wherein residue 111 is S or T
wherein residue 112 is A or E or G or K or P
wherein residue 113 is D or E or K or S
wherein residue 115 is E or V
wherein residue 116 is R or Q or E or K or P or T or V
wherein residue 117 is I or L or V
wherein residue 118 is A or R or S
wherein residue 120 is L or F or V
wherein residue 122 is A or T
wherein residue 129 is A or T
wherein residue 130 is A or N or Q or E or G or L or K or M or T or Y
wherein residue 131 is D or E or L or P or S
wherein residue 133 is A or T
wherein residue 134 is R or D or Q or E or K
wherein residue 135 is R or E or K or S
wherein residue 137 is N or G
wherein residue 138 is I or L
wherein residue 139 is R or K or P
wherein residue 140 is R or L or P
wherein residue 144 is Q or M
wherein residue 146 is G or S
wherein residue 152 is H or L
wherein residue 153 is A or C
wherein residue 155 is A or V
wherein residue 156 is C or S
wherein residue 159 is A or R or D or Q or E or H or L or K
wherein residue 161 is R or D or E or I or L
wherein residue 162 is A or R or D or Q or E or K
wherein residue 163 is A or R or D or Q or L or K
wherein residue 165 is I or L or W or Y
wherein residue 167 is N or D
wherein residue 168 is L or P
wherein residue 169 is A or N or D or E or S or T
wherein residue 170 is N or D or S or T
wherein residue 171 is N or Q or K or P or S or T
wherein residue 172 is R or E or H or L or K or S or T or W
wherein residue 173 is A or R or N or D or G or K or P
wherein residue 174 is D or E or L or P
wherein residue 175 is D or E
wherein residue 176 is A or D or Q or E or K or T
wherein residue 177 is Q or P
wherein residue 178 is A or V
wherein residue 179 is E or K or P or S or V
wherein residue 181 is A or L or F or Y
wherein residue 183 is R or Q or E or H or I or L or M or F or W or Y
wherein residue 185 is R or L or K or T
wherein residue 186 is A or R or N or W or V
wherein residue 187 is A or R or G or K
wherein residue 190 is R or L or K
wherein residue 191 is R or K or S
wherein residue 196 is D or I or K or S or W
wherein residue 197 is D or Q
wherein residue 199 is A or S
wherein residue 204 is D or E or G or M or S
wherein residue 207 is I or T or V
wherein residue 208 is E or K or T
wherein residue 209 is A or Q or E or M or T
wherein residue 211 is R or K
wherein residue 212 is A or R or N or Q or E or L or K
wherein residue 213 is A or S
wherein residue 216 is I or V
wherein residue 221 is A or F
wherein residue 232 is I or V
wherein residue 233 is R or Q or I or W
wherein residue 243 is I or L
wherein residue 249 is L or F or Y
wherein residue 250 is R or T
wherein residue 254 is S or T
wherein residue 259 is E or T
wherein residue 261 is R or P
wherein residue 262 is S or T
wherein residue 263 is A or F or T or V
wherein residue 264 is A or F
wherein residue 265 is A or D or Q or E or P
wherein residue 268 is A or D
wherein residue 269 is R or Q or G or K or T
wherein residue 271 is A or P
wherein residue 272 is A or D or E or K or P
wherein residue 273 is R or N or E or G or K or S
wherein residue 276 is L or V
wherein residue 279 is G or S
wherein residue 285 is Q or E or S
wherein residue 286 is I or L or M or F or V
wherein residue 287 is D or E or S or T
wherein residue 288 is A or R or E or L or K or P or V
wherein residue 289 is A or D or E or K
wherein residue 290 is D or Q or E or T
wherein residue 291 is A or F
wherein residue 292 is L or K
wherein residue 293 is A or R or N or E or K or T
wherein residue 294 is I or L
wherein residue 295 is A or L or M or V
wherein residue 296 is R or D or Q or E or H or K
wherein residue 299 is R or I or K or V
wherein residue 300 is A or R or D or E or L or K
wherein residue 302 is E or G or K
wherein residue 303 is Q or E or H or I or L or K or V
wherein residue 304 is R or N or K or P or S
wherein residue 305 is A or F or T or V
wherein residue 314 is I or V
wherein residue 317 is A or R or Q or E or H or L or K or S or Y
wherein residue 318 is A or R or D or E or K or S or T
wherein residue 320 is I or L or T or V
wherein residue 321 is D or E or S
wherein residue 322 is A or D or I or L or F or P or S or V
wherein residue 323 is I or L
wherein residue 325 is D or E or P
wherein residue 327 is R or L or K or F or Y
wherein residue 328 is G or L or M or F
wherein residue 330 is D or E or P
wherein residue 331 is A or R or N or Q or L or K
wherein residue 332 is G or I
wherein residue 334 is I or V
wherein residue 338 is A or C
wherein residue 347 is R or E or G or K or P
wherein residue 362 is L or M or V
wherein residue 364 is A or S
wherein residue 373 is C or F
wherein residue 375 is D or P
wherein residue 385 is A or R or T or V
wherein residue 388 is A or S or T or V
wherein residue 389 is R or N or D or E or K
wherein residue 390 is E or I or K or V
wherein residue 391 is R or L or K
wherein residue 392 is R or N or E or G or H or K
wherein residue 393 is I or V
wherein residue 395 is I or V
wherein residue 398 is R or D or E
wherein residue 399 is R or N or D or E or K
wherein residue 401 is F or W
wherein residue 402 is R or N or D or Q or E or I or K or V
wherein residue 403 is A or R or E or K or P or T or V
wherein residue 404 is A or R or D or Q or E or G or K
wherein residue 405 is A or R or N or D or E or K
wherein residue 406 is I or V
wherein residue 407 is A or I or V
wherein residue 408 is A or R or N or D or E or K
wherein residue 410 is A or I or L or V
wherein residue 411 is R or N or Q or E or H or K
wherein residue 412 is R or E or H or L or K or T or Y
wherein residue 413 is I or V
wherein residue 414 is L or M
wherein residue 415 is R or Q or E or L or K or T or V
wherein residue 416 is A or D or E or S
wherein residue 417 is A or E or K or P
wherein residue 418 is A or R or E or K or F or S
wherein residue 420 is A or R or Q or E or L or K
wherein residue 421 is A or E or G or H or K or P or Y or V
wherein residue 422 is I or M or Y
wherein residue 423 is A or R or K
wherein residue 424 is A or R or N or D or Q or E or K
wherein residue 425 is R or N
wherein residue 427 is A or R or Q or E or L or K
wherein residue 428 is A or R or E or L or K or V
wherein residue 430 is A or K
wherein residue 431 is A or R or D or Q or E
wherein residue 433 is A or L
wherein residue 435 is R or G or V
wherein residue 437 is I or L
wherein residue 438 is A or M
wherein residue 439 is K or P
wherein residue 449 is R or S
wherein residue 451 is Q or V (SEQ ID NO: 518).

7. The polypeptide of any one of claims 1-6, wherein the polypeptide is expressed in a host microorganism.

8. The polypeptide of claim 7, wherein the host microorganism is E. coli, Saccharomyces sp., Aspergillus sp., Pichia sp., or Bacillus sp.

9. The polypeptide of any one of claims 1-8, wherein the polypeptide is immobilized to a solid support.

10. The polypeptide of claim 9, wherein the solid support is derivatized from cellulose, glass, ceramic, methacrylate, styrene, acrylic, a metal oxide, or a membrane.

11. The polypeptide of claim 9 or 10, wherein the polypeptide is immobilized to the solid support by covalent attachment, adsorption, cross-linking, entrapment, or encapsulation.

12. A method for transferring a sugar moiety to a substrate steviol glycoside, the method comprising contacting a beta-1,3-glycosyltransferase polypeptide, a sucrose synthase and optionally a beta-1,2-glycosyltransferase polypeptide with a starting composition comprising one or more steviol glycosides, a nucleotide diphosphate, and sucrose.

13. The method of claim 12, wherein the beta-1,3-glycosyltransferase polypeptide is an engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to an amino acid sequence selected from the group consisting of SEQ ID NOs: 4-517, 2, and 3.

14. The method of claim 12, wherein the sucrose synthase is an engineered sucrose synthase with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to a sucrose synthase derived from the species Acidithiobacillus caldus, Nitrosomonas europaea, Denitrovibrio acetiphilus, Thermosynechococcus elongatus, Oryza sativa, Arabidopsis thaliana, or Coffea arabica.

15. The method of claim 12, wherein the beta-1,2-glycosyltransferase polypeptide is an engineered beta-1,2-glycosyltransferase polypeptide with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to a beta-1,2-glycosyltransferase polypeptide derived from the species Hordeum vulgare, Oryza sativa, Solanum lycopersicum, Lycium barbarum, Solanum tuberosum, or Stevia rebaudiana.

16. The method of claim 12, wherein
(a) the beta-1,3-glycosyltransferase polypeptide is an engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to an amino acid sequence selected from the group consisting of SEQ ID NOs: 4-517, 2, and 3.
(b) the sucrose synthase is an engineered sucrose synthase with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to a sucrose synthase derived from the species Acidithiobacillus caldus, Nitrosomonas europaea, Denitrovibrio acetiphilus, Thermosynechococcus elongatus, Oryza sativa, Arabidopsis thaliana, or Coffea arabica.
(c) the beta-1,2-glycosyltransferase polypeptide is an engineered beta-1,2-glycosyltransferase polypeptide with a polypeptide sequence that is at least 60%, 65%, 70%, 75%, 80%, 85%, 86%, 87%, 88%, 89%, 90%, 91%, 92%, 93%, 94%, 95%, 96%, 97%, 98%, 99%, or more sequence identity to a beta-1,2-glycosyltransferase polypeptide derived from the species Hordeum vulgare, Oryza sativa, Solanum lycopersicum, Lycium barbarum, Solanum tuberosum, or Stevia rebaudiana.

17. The method of claim 12, wherein the beta-1,3-glycosyltransferase polypeptide is an engineered beta-1,3-glycosyltransferase polypeptide that has a score greater than 332 when scored by the PSSM shown in Table 8, wherein the engineered beta-1,3-glycosyltransferase polypeptide has been aligned to SEQ ID NO: 2 prior to scoring.

18. The method of claim 12, wherein the beta-1,3-glycosyltransferase polypeptide is an engineered beta-1,3-glycosyltransferase polypeptide that has a score greater than 54.5 when scored by the PSSM shown in Table 9, wherein the engineered beta-1,3-glycosyltransferase polypeptide has been aligned to SEQ ID NO: 2 prior to scoring.

19. The method of claim 12, wherein the beta-1,3-glycosyltransferase polypeptide is an engineered beta-1,3-glycosyltransferase polypeptide that has a score greater than 58.3 when scored by the PSSM shown in Table 10, wherein the engineered beta-1,3-glycosyltransferase polypeptide has been aligned to SEQ ID NO: 2 prior to scoring.

20. The method of any one of claims 12 to 19, wherein the substrate steviol glycoside comprises at least one steviol glycoside selected from the group consisting of steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside H, rebaudioside L, rebaudioside K, rebaudioside J, rebaudioside M, rebaudioside D, rebaudioside E2, rebaudioside AM, rebaudioside N, rebaudioside 0, rebaudioside Q, an isomer thereof, and a synthetic steviol glycoside.

21. The method of claim 20, wherein the substrate steviol glycoside comprises a mixture of stevioside and rebaudioside A.

22. The method of claim 20, wherein the substrate steviol glycoside comprises stevioside.

23. The method of claim 20, wherein the substrate steviol glycoside comprises one or more selected from the group consisting of: Reb A, RS50, RS60, RS90, and RS99.

24. The method of any one of claims 12 to 23, further comprising producing a target steviol glycoside, wherein the target steviol glycoside comprises at least one steviol glycoside selected from the group consisting of steviol, steviol-13-O-glucoside, steviol-19-O-glucoside, rubusoside, steviol-1,2-bioside, steviol-1,3-bioside, rubusoside, dulcoside B, dulcoside A, rebaudioside B, rebaudioside G, stevioside, rebaudioside C, rebaudioside F, rebaudioside A, rebaudioside I, rebaudioside E, rebaudioside E2, rebaudioside H, rebaudioside L, rebaudioside K, rebaudioside J, rebaudioside AM, rebaudioside M, rebaudioside D, rebaudioside N, rebaudioside O, rebaudioside Q, an isomer thereof, and a synthetic steviol glycoside.

25. The method of claim 24, wherein the target steviol glycoside comprises at least one steviol glycoside selected from the group consisting of stevioside rebaudioside A, rebaudioside E, rebaudioside E2, rebaudioside I, rebaudioside D, rebaudioside AM, and rebaudioside M.

26. The method of claim 24, wherein the target steviol glycoside comprises at least one steviol glycoside selected from the group consisting of rebaudioside I, rebaudioside D, rebaudioside AM, and rebaudioside M.

27. The method of claim 24, wherein the target steviol glycoside comprises rebaudioside M.

28. The method of claim 24, wherein the target steviol glycoside comprises rebaudioside AM.

29. The method of any of claims 12 to 28, wherein the nucleotide diphosphate comprises one selected from the group consisting of adenosine diphosphate, uridine diphosphate, cytidine diphosphate, thymidine diphosphate, and guanosine diphosphate.

30. The method of any of claims 12 to 28, wherein the nucleotide diphosphate comprises one selected from the group consisting of adenosine diphosphate, guanosine diphosphate, cytidine diphosphate, or thymidine diphosphate.

31. The method of any of claims 12 to 28, wherein the nucleotide diphosphate comprises adenosine diphosphate.

32. The method of any of claims 12 to 31, wherein the starting composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of steviol glycoside by weight on an anhydrous basis.

33. The method of any of claims 12 to 32, further comprising producing a target composition, wherein the target composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of the target steviol glycoside by weight on an anhydrous basis.

34. A polynucleotide encoding a polypeptide of any of claims 1 to 6.

35. A host microorganism heterologously expressing a polynucleotide of claim 34.

36. A microorganism of claim 35 where the host microorganism is E. coli, Saccharomyces sp., Aspergillus sp., Pichia sp., or Bacillus sp.

37. A method for transferring a sugar moiety to a substrate steviol glycoside, the method comprising contacting a beta-1,3-glycosyltransferase polypeptide and a sucrose synthase with one or more steviol glycosides, a nucleotide diphosphate, and sucrose.

38. The polypeptide of claim 1, wherein the polypeptide comprises H at position 24, D at position 123, D at position 379, and Q at position 380, and wherein the position numbers are based on SEQ ID NO: 2.

39. An engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 4, and wherein the polypeptide comprises H at position 24, D at position 123, D at position 379, and Q at position 380.

40. An engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 3, and wherein the polypeptide comprises H at position 24, D at position 123, D at position 379, and Q at position 380.

41. An engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 219, and wherein the polypeptide comprises H at position 24, D at position 123, D at position 379, and Q at position 380.

42. An engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 208, and wherein the polypeptide comprises H at position 24, D at position 123, D at position 379, and Q at position 380.

43. An engineered beta-1,3-glycosyltransferase polypeptide that comprises an amino acid sequence that is at least 60%, at least 65%, at least 70%, at least 75%, at least 80%, at least 85%, at least 90%, at least 95%, at least 98%, at least 99%, or 100% identical to SEQ ID NO: 181, and wherein the polypeptide comprises H at position 24, D at position 123, D at position 379, and Q at position 380.
